(19) United States
(12) Patent Application Publication
(10) Pub. No.: US 2022/0119825 A1
(43) Pub. Date: Apr. 21, 2022

(54) SCALABLE PEPTIDE-GPCR INTERCELLULAR SIGNALING SYSTEMS

(71) Applicant: THE TRUSTEES OF COLUMBIA UNIVERSITY IN THE CITY OF NEW YORK, New York, NY (US)

(72) Inventors: Virginia Cornish, New York, NY (US); James Brisbois, Cambridge, MA (US); Sonja Billerbeck, Groningen (NL); Miguel Jimenez, Winthrop, MA (US)

(73) Assignee: THE TRUSTEES OF COLUMBIA UNIVERSITY IN THE CITY OF NEW YORK, New York, NY (US)

(21) Appl. No.: 17/514,648

(22) Filed: Oct. 29, 2021

Related U.S. Application Data
(63) Continuation of application No. PCT/US2020/030795, filed on Apr. 30, 2020.
(60) Provisional application No. 62/840,812, filed on Apr. 30, 2019.

(51) Int. Cl. C12N 15/81; C07K 14/38; C07K 14/39

(52) U.S. Cl. CPC C12N 15/81; C12N 1/16; C07K 14/39; C07K 14/38

(57) ABSTRACT

The present disclosure relates to intercellular signaling between genetically-engineered cells and, more specifically, to a scalable peptide-GPCR intercellular signaling system. The present disclosure provides an intercellular signaling system that includes at least two cells that have been genetically-engineered to communicate with each other, methods of use and kits thereof.

### CROSS-REFERENCE TO RELATED APPLICATIONS

This application is a continuation of International Patent Application No. PCT/US2020/030795, filed Apr. 30, 2020, which claims priority to U.S. Provisional Application No. 62/840,812, filed on Apr. 30, 2019, the contents of each of which are incorporated by reference in their entireties, and to each of which priority is claimed.

### STATEMENT REGARDING FEDERALLY SPONSORED RESEARCH OR DEVELOPMENT

This invention was made with government support under AI110794, GM066704, RR027050 awarded by the National Institutes of Health, 1144155 awarded by the National Science Foundation, and HR0011-15-2-0032 awarded by DOD/DARPA. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 27, 2021, is named 070050 6561 SL.txt and is 434,557 bytes in size.

### TECHNICAL FIELD

The present disclosure relates to intercellular signaling pathways between genetically-engineered cells and, more specifically, to a scalable G-protein coupled receptor (GPCR)-ligand intercellular signaling system.

### BACKGROUND

Genetic engineering techniques have been applied to create specialized biological systems from living cells. However, the development of higher-order cellular networks responsive to signals in a coordinated fashion has been hampered due to a need for an adaptable cell signaling language. Certain approaches based on quorum sensing or synthetic receptors are not scalable, and are not necessarily suitable for long-range communication between cells. Therefore, an improved versatile, scalable intercellular signaling language for cell-cell communication is needed.

### SUMMARY

The present disclosure provides a genetically-engineered cell that expresses at least one heterologous G-protein coupled receptor (GPCR) and/or at least one heterologous secretable GPCR peptide ligand. For example, but not by way of limitation, a genetically-engineered cell can express at least one heterologous GPCR, express at least one secretable GPCR peptide ligand or express at least one heterologous GPCR and at least one secretable GPCR peptide ligand. In certain embodiments, the amino acid sequence of the heterologous GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211. In certain embodiments, the amino acid sequence of the GPCR peptide ligand is at least about 75% homologous to an amino acid sequence comprising any one of SEQ ID NOs: 1-116 or an amino acid sequence provided in Table 12 and/or encoded by a nucleotide sequence that is about 75% homologous to a nucleotide sequence comprising any one of SEQ ID NOs: 215-230. In certain embodiments, the secretable GPCR ligand and/or the heterologous GPCR are identified and/or derived from a eukaryotic organism, e.g., a yeast. In certain embodiments, the heterologous GPCR is selectively activated by a ligand, e.g., a peptide, a protein or portion thereof, a toxin, a small molecule, a nucleotide, a lipid, a chemical, a photon, an electrical signal or a compound. In certain embodiments, the ligand is a peptide.

The present disclosure further provides an intercellular signaling system that includes two or more, three or more, four or more or five or more genetically-engineered cells disclosed herein. In certain embodiments, an intercellular signaling system of the present disclosure includes a first genetically-engineered cell expressing at least one secretable G-protein coupled receptor (GPCR) ligand and a second genetically-engineered cell expressing at least one heterologous GPCR. In certain embodiments, the amino acid sequence of the heterologous GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211. In certain embodiments, the amino acid sequence of the secretable GPCR ligand is at least about 75% homologous to an amino acid sequence comprising any one of SEQ ID NOs: 1-116 or an amino acid sequence provided in Table 12 and/or is encoded by a nucleotide sequence that is about 75% homologous to a nucleotide sequence comprising any one of SEQ ID NOs: 215-230. In certain embodiments, the secretable GPCR ligand and/or the heterologous GPCR are identified and/or derived from a eukaryotic organism. In certain embodiments, the secretable GPCR ligand is selected from the group consisting of a protein or portion thereof and a peptide. In certain embodiments, the secretable GPCR ligand of the first genetically-engineered cell selectively activates the heterologous GPCR of the second genetically-engineered cell. Alternatively, the secretable GPCR ligand of the first genetically-engineered cell does not activate the heterologous GPCR of the second genetically-engineered cell. For example, but not by way of limitation, the heterologous GPCR of the second genetically-engineered cell is activated by an exogenous ligand, e.g., a peptide, a protein or portion thereof, a toxin, a small molecule, a nucleotide, a lipid, chemicals, a photon, an electrical signal and a compound.

In certain embodiments, the second genetically-engineered cell further expresses at least one secretable GPCR ligand and/or the first genetically-engineered cell further expresses at least one heterologous GPCR. For example, but not by way of limitation, the first genetically-engineered cell of an intercellular signaling system expresses at least one secretable GPCR ligand and at least one heterologous GPCR. In certain embodiments, the second genetically-engineered cell of such a system expresses at least one secretable GPCR ligand and at least one heterologous GPCR. In certain embodiments, the secretable GPCR ligand expressed by the second genetically-engineered cell is different from the secretable GPCR ligand expressed by the first genetically-engineered cell, e.g., selectively activate different GPCRs. In certain embodiments, the heterologous GPCR expressed by the first genetically-engineered cell is different from the heterologous GPCR expressed by the second genetically-engineered cell, e.g., are selectively activated by different ligands. In certain embodiments, the secretable GPCR ligand expressed by the second genetically-engineered cell does not activate the heterologous GPCR expressed by the second genetically-engineered cell. In certain embodiments, the secretable GPCR ligand expressed by the first genetically-engineered cell does not activate the heterologous GPCR expressed by the first genetically-engineered cell. In certain embodiments, the secretable GPCR ligand of the first genetically-engineered cell selectively activates the heterologous GPCR of the second genetically-engineered cell. In certain embodiments, the secretable GPCR ligand of the first genetically-engineered cell does not activate the heterologous GPCR of the second genetically-engineered cell. In certain embodiments, the secretable GPCR ligand expressed by the second genetically-engineered cell selectively activates the heterologous GPCR expressed by the first genetically-engineered cell. In certain embodiments, the secretable GPCR ligand expressed by the second genetically-engineered cell does not activate the heterologous GPCR expressed by the first genetically-engineered cell. In certain embodiments, the secretable GPCR ligand expressed by the second genetically-engineered cell and/or the first genetically-engineered cell selectively activates a GPCR expressed on a third cell.

In certain embodiments, one or more endogenous GPCR genes and/or endogenous GPCR ligand genes of one or more genetically-engineered cells disclosed herein, e.g., the first genetically-engineered cell and/or the second genetically-engineered cell, are knocked out. In certain embodiments, one or more of the genetically-engineered cells disclosed herein, e.g., the first genetically-engineered cell and/or the second genetically-engineered cell, further include a nucleic acid that encodes a sensor and/or a nucleic acid that encodes a detectable reporter. In certain embodiments, one or more of the genetically-engineered cells disclosed herein, e.g., the first genetically-engineered cell and/or the second genetically-engineered cell, further include a nucleic acid that encodes a product of interest.

In certain embodiments, an intercellular signaling system of the present disclosure further includes a third genetically-engineered, a fourth genetically-engineered cell, a fifth genetically-engineered cell, a sixth genetically-engineered cell, a seventh genetically-engineered cell and/or an eighth genetically-engineered cell or more. In certain embodiments, each genetically-engineered cell expresses at least one heterologous GPCR and/or at least one secretable GPCR ligand. In certain embodiments, each of the heterologous GPCRs are different, e.g., are selectively activated by different ligands, and/or each of the secretable GPCR ligands are different, e.g., selectively activate different GPCRs. Alternatively and/or additionally, one or more heterologous GPCRs are the same and/or one or more of the secretable GPCR ligands are the same.

The present disclosure further provides for an intercellular signaling system that includes a first genetically-engineered cell including: (i) a nucleic acid encoding a first heterologous G-protein coupled receptor (GPCR); and/or (ii) a nucleic acid encoding a first secretable GPCR ligand; and a second genetically-engineered cell including: (i) a nucleic acid encoding a second heterologous GPCR; and/or (ii) a nucleic acid encoding a second secretable GPCR ligand. In certain embodiments, the first GPCR and/or the second GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211. In certain embodiments, the first and/or second secretable GPCR peptide ligand is at least about 75% homologous to an amino acid sequence comprising any one of SEQ ID NOs: 1-116 or an amino acid sequence provided in Table 12 and/or is encoded by a nucleotide sequence that is about 75% homologous to a nucleotide sequence comprising any one of SEQ ID NOs: 215-230. In certain embodiments, the first secretable GPCR ligand of the first genetically-engineered cell selectively activates the second heterologous GPCR of the second genetically-engineered cell, the second secretable GPCR ligand of the second genetically-engineered cell selectively activates the first heterologous GPCR of the first genetically-engineered cell, the second secretable GPCR ligand of the second genetically-engineered cell selectively does not activate the first heterologous GPCR of the first genetically-engineered cell and/or the first heterologous GPCR and the second heterologous GPCR are selectively activated by different ligands.

In certain embodiments, the intercellular signaling system further includes a third genetically-engineered cell that includes a nucleic acid encoding a third heterologous GPCR; and/or a nucleic acid encoding a third secretable GPCR ligand. In certain embodiments, the first secretable GPCR ligand of the first genetically-engineered cell selectively activates the third heterologous GPCR of the third genetically-engineered cell and/or the second heterologous GPCR of the second genetically-engineered cell. In certain embodiments, the second secretable GPCR ligand of the second genetically-engineered cell selectively activates the third heterologous GPCR of the third genetically-engineered cell and/or the first heterologous GPCR of the first genetically-engineered cell. In certain embodiments, the third secretable GPCR ligand of the third genetically-engineered cell selectively activates the first heterologous GPCR of the first genetically-engineered cell and/or the second heterologous GPCR of the third genetically-engineered cell. In certain embodiments, the third secretable GPCR ligand of the third genetically-engineered cell does not activate the third heterologous GPCR of the third genetically-engineered cell. In certain embodiments, the first secretable GPCR ligand of the first genetically-engineered cell does not activate the first heterologous GPCR of the first genetically-engineered cell. In certain embodiments, the second secretable GPCR ligand of the second genetically-engineered cell does not activate the second heterologous GPCR of the second genetically-engineered cell.

The present disclosure further provides a kit that includes a genetically modified cell or an intercellular signaling system as disclosed herein. For example, but not by way of limitation, the genetically modified cell present within a kit of the present disclosure includes at least one heterologous G-protein coupled receptor (GPCR) and/or at least one heterologous secretable GPCR peptide ligand. In certain embodiments, the intercellular signaling system present within a kit of the present disclosure includes a first genetically-engineered cell expressing at least one secretable G-protein coupled receptor (GPCR) ligand; and a second genetically-engineered cell expressing at least one heterologous GPCR. Alternatively and/or additionally, the intercellular signaling system to be included in a kit of the present disclosure includes a first genetically-engineered cell that includes (i) a nucleic acid encoding a first heterologous G-protein coupled receptor (GPCR); and/or (ii) a nucleic acid encoding a first secretable GPCR ligand; and a second genetically-engineered cell that includes (i) a nucleic acid encoding a second heterologous GPCR; and/or (ii) a nucleic acid encoding a second secretable GPCR ligand. In certain embodiments, the amino acid sequence of the heterologous GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211. In certain embodiments, the amino acid sequence of the GPCR ligand or GPCR peptide ligand is at least about 75% homologous to an amino acid sequence comprising any one of SEQ ID NOs: 1-116 or an amino acid sequence provided in Table 12 and/or encoded by a nucleotide sequence that is about 75% homologous to a nucleotide sequence comprising any one of SEQ ID NOs: 215-230.

In another aspect, the present disclosure provides an intercellular signaling system for spatial control of gene expression and/or temporal control of gene expression, for the generation of pharmaceuticals and/or therapeutics, for performing computations, as a biosensor and for the generation of a product of interest. In certain embodiments, the intercellular signaling system includes a first genetically-engineered cell expressing at least one secretable G-protein coupled receptor (GPCR) ligand; and a second genetically-engineered cell expressing at least one heterologous GPCR. In certain embodiments, the intercellular signaling system includes a first genetically-engineered cell including: (a) a nucleic acid encoding a first heterologous G-protein coupled receptor (GPCR); and/or (b) a nucleic acid encoding a first secretable GPCR ligand; and a second genetically-engineered cell including: (a) a nucleic acid encoding a second heterologous GPCR; and/or (b) a nucleic acid encoding a second secretable GPCR ligand. In certain embodiments, the amino acid sequence of the heterologous GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211. In certain embodiments, the amino acid sequence of the secretable GPCR ligand is at least about 75% homologous to an amino acid sequence comprising any one of SEQ ID NOs: 1-116 or an amino acid sequence provided in Table 12 and/or is encoded by a nucleotide sequence that is about 75% homologous to a nucleotide sequence comprising any one of SEQ ID NOs: 215-230

In certain embodiments, the genetically-engineered cells disclosed herein are independently selected from the group consisting of a mammalian cell, a plant cell and a fungal cell. For example, but not by way of limitation, the genetically-engineered cells are fungal cells, fungal cells from the phylum Ascomycota and/or fungal cells independently selected from the group consisting of Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, Capronia coronate and combinations thereof.

In certain embodiments, an intercellular signaling system of the present disclosure has a topology selected from the group consisting of a daisy chain network topology, a bus type network topology, a branched type network topology, a ring network topology, a mesh network topology, a hybrid network topology, a star type network topology and a combination thereof.

In certain embodiments, the product of interest is selected from the group consisting of hormones, toxins, receptors, fusion proteins, regulatory factors, growth factors, complement system factors, enzymes, clotting factors, anti-clotting factors, kinases, cytokines, CD proteins, interleukins, therapeutic proteins, diagnostic proteins, enzymes, biosynthetic pathways, antibodies and combinations thereof.

In another aspect, the present disclosure provides a method for the identification of a G-protein coupled receptor (GPCR) and/or a GPCR ligand to be expressed in a genetically-engineered cell. In certain embodiments, the method for identifying a GPCR includes searching a protein and/or genomic database and/or literature for a protein and/or a gene with homology to: (i) a S. cerevisiae Ste2 receptor and/or Ste3 receptor; (ii) a GPCR having an amino acid sequence comprising any one of SEQ ID NOs: 117-161; (iii) a GPCR having an amino acid sequence provided in Table 11; and/or (iv) a GPCR encoded by a nucleotide sequence comprising any one of SEQ ID NOs: 168-211. In certain embodiments, the method for identifying a GPCR ligand includes searching a protein and/or genomic database and/or literature for a protein, peptide and/or a gene with homology to: (i) a GPCR peptide ligand having an amino acid sequence comprising any one of SEQ ID NOs: 1-116; (ii) a GPCR peptide ligand comprising an amino acid sequence provided in Table 12; (iii) a GPCR peptide ligand encoded by a nucleotide sequence comprising any one of SEQ ID NOs: 215-230 to identify a GPCR ligand; and/or (iv) a yeast pheromone or a motif thereof. The present disclosure further provides a genetically-engineered cell that expresses a GPCR and/or GPCR ligand identified by the methods disclosed herein.

### BRIEF DESCRIPTION OF THE DRAWINGS

FIG. 1A provides a schematic showing an exemplary language component acquisition pipeline—Genome mining yields a scalable pool of peptide/GPCR interfaces for synthetic communication. Pipeline for component harvest and communication assembly.

FIG. 1B provides a schematic showing an example of how GPCRs and peptides can be swapped by simple DNA cloning. Conservation in both GPCR signal transduction and peptide secretion permits scalable communication without any additional strain engineering.

FIG. 1C provides a schematic showing exemplary genome-mined peptide/GPCR functional pairs in yeast. GPCR nomenclature corresponds to species names (Table 3). Experiments were performed in triplicate and full data sets with errors (standard deviations) and individual data points are given in FIG. 18.

FIGS. 2A-2C provide schematics showing exemplary conserved motifs reported to be important for signaling. Sequence logos were generated using multiple sequence alignments generated with Clustal Omega (Sievers, F. et al. Fast, scalable generation of high-quality protein multiple sequence alignments using Clustal Omega. Mol Syst Biol 7 (2011)) and using the WebLogo online tool (Crooks, G. E., Hon, G., Chandonia, J. M. & Brenner, S. E. WebLogo: A sequence logo generator. Genome Res 14, 1188-1190 (2004)). Numbering refers to the amino acid residue in the S. cerevisiae Ste2.

FIG. 3 provides graphs reporting exemplary verification of the peptide/GPCR language in a- and alpha-mating types. Dose responses to the appropriate synthetic peptide are shown. Fluorescence was recorded after 12 hours of incubation and experiments were run in triplicates.

FIGS. 4A-4D provide graphs reporting examples of basal and maximal activation levels of functional, constitutive and non-functional peptide/GPCR pairs. JTy014 was transformed with the appropriate GPCR expression construct. Cells were cultured in the absence or presence of 40 μM cognate synthetic peptide ligand. The peptide sequence #1 (Table 3, Table 4) was used for each GPCR. OD₆₀₀ and Fluorescence was recorded after 8 hours. The peptide sequences #2 and #3 represent alternative peptides. Experiments were performed in 96-well plates (200 μl total culture volume) and experiments were run in triplicates. FIG. 4A: Functional peptide/GPCR pairs. FIG. 4B: Constitutive GPCRs and their additional activation by cognate peptide ligand. FIG. 4C: Non-functional peptide/GPCR pairs. FIG. 4D: Activation of non-functional GPCRs by alternative peptide ligands (Table 3, Table 4).

FIG. 5A provides a schematic of an exemplary framework for GPCR characterization. Parameter values for basal and maximal activation, fold change, EC50, dynamic range (given through Hill slope) were extracted by fitting each curve to a four-parameter nonlinear regression model using PRISM GraphPad. Experiments were done in triplicates and errors represent the standard deviation.

FIG. 5B provides an exemplary graph showing GPCRs cover a wide range of response parameters. The EC₅₀ values of peptide/GPCR pairs are plotted against fold change in activation. Experiments were done in triplicate and parameter errors can be found in Table 6.

FIG. 5C provides an exemplary schematic showing GPCRs are naturally orthogonal across non-cognate synthetic peptide ligands. GPCRs are organized according to a phylogenetic tree of the protein sequences.

FIG. 5D provides a schematic reporting exemplary orthogonality of peptide/GPCR pairs when peptides are secreted. 15 exemplary best performing pairs (marked in red in panels a-c) were chosen for secretion. Experiments were performed by combinatorial co-culturing of strains constitutively secreting one of the indicated peptides and strains expressing one of the indicated GPCRs using GPCR-controlled fluorescent as read-out. Experiments were performed in triplicate and results represent the mean.

FIG. 6 provides graphs reporting dose response curves for exemplary functional peptide/GPCR pairs. Strain JTy014 was transformed with the appropriate GPCR expression constructs. Each strain was tested with its cognate synthetic peptide. GPCR activation was monitored by activation of a red fluorescent reporter gene under the control of the FUS1 promoter. Data were collected after 8 hours. Experiments were run in triplicates.

FIG. 7 provides graphs reporting exemplary GPCR response behavior on single cell level when expressed from plasmids or when integrated into the chromosome (Ste2 locus). Flow cytometry was used to investigate the response behavior for three GPCRs on single cell level when exposed to increasing concentrations of their corresponding peptide ligand. For each sample, 50,000 cells were analyzed using a BD LSRII flow cytometer (excitation: 594 nm, emission: 620 nm). The fluorescence values were normalized by the forward scatter of each event to account for different cell size using FlowJo Software. Data of a single experiment are shown, but data were reproduced several times.

FIGS. 8A-8C provide graphs reporting exemplary reversibility and re-inducibility of GPCR signaling.

FIG. 9 provides graphs reporting exemplary co-expression of two orthogonal GPCRs and single/dual response characteristics.

FIG. 10 provides a schematic showing examples of 17 receptors that are fully orthogonal and not activated by the other 16 non-cognate peptide ligands. Data shown in this Figure were extracted from FIG. 5C.

FIG. 11 provides a graph reporting exemplary results of an on/off screen for 19 GPCRs and their alternative near-cognate peptide ligand candidates. Numbering of the near-cognate peptide ligand candidates corresponds to Table 4. Red arrows indicate GPCRs that were not activated by all tested alternative peptide ligand candidates.

FIG. 12 provides graphs reporting exemplary dose response of GPCRs to their alternative near-cognate peptide ligand candidates.

FIG. 13 is a graph reporting exemplary dose response of Ca. Ste2 using alanine-scanned peptide ligands. Strain JTy014 was transformed with the Ca.Ste2 expression construct. The resulting strain was tested with the indicated synthetic peptide ligands. GPCR activation was monitored by activation of a red fluorescent reporter gene under the control of the FUS1 promoter. Data were collected after 12 hours. Experiments were run in triplicates.

FIGS. 14A-14D provide graphs reporting exemplary dose responses of promiscuous GPCRs and their cognate or non-cognate peptide ligands. Strain JTy014 was transformed with the appropriate GPCR expression constructs. Each strain was tested with its cognate synthetic peptide ligand #1 and its non-orthogonal non-cognate peptide ligands as indicated. GPCR activation was monitored by activation of a red fluorescent reporter gene under the control of the FUS1 promoter. Data were collected after 12 hours. Experiments were run in triplicates.

FIGS. 15A-15C provide schematics showing exemplary peptide acceptor vector design. FIG. 15A provides a schematic representation of the S. cerevisiae alpha-factor precursor architecture with the secretion signal (blue), Kex2 (grey) and Ste13 (orange) processing sites and three copies of the peptide sequence (red). FIG. 15B provides an overview on pre-pro-peptide processing, resulting in mature alpha-factor. FIG. 15C provides a schematic representation of the peptide acceptor vector. The peptide expression cassette includes either a constitutive promoter (ADH1p) or a peptide-dependent promoter (FUS1p or FIG1p), the alpha-factor pro sequence with or without the Ste13 processing site, a unique (AflII) restriction site for peptide swapping and a CYC1 terminator.

FIG. 16 provides a graph reporting exemplary data of secretion of peptide ligands with and without Ste13 processing site. Peptide expression cassettes with and without the Ste13 processing site (EAEA) were cloned under control of the constitutive ADH1 promoter. Peptide expression constructs were used to transform strain yNA899 and the resulting strains were co-cultured with a sensing strain expressing the cognate GPCR and a fluorescent read-out. Secretion and Sensing strains were co-cultured 1:1 in 96-well plates (200 μl total culturing volume) and fluorescence was measured after 12 hours. Experiments were run in triplicates. An unpaired t-test was performed for each peptide with an alpha value=0.05. A single asterisk indicates a P value <0.05; a double asterisk indicates a P value <0.01. For simplicity, all peptide constructs eventually used herein contained the Ste13 processing site.

FIG. 17 provides images of an exemplary fluorescent halo assay for 16 peptide-secreting strains. Sensing strains for all 16 peptides carrying a pheromone induced red fluorescent reporter, were spread on SC plates. Secreting strains were dotted on the sensing strains in the pattern depicted in scheme bellow. The appearance of a halo around the dot is an indication for secretion of the peptide. All peptides except for Le show a halo. Data of a single experiment are shown.

FIG. 18A provides a schematic showing an exemplary minimal two-cell communication links.

FIG. 18B provides a schematic showing exemplary functional transfer of information through all 56 two-cell communication links established from eight peptide/GPCR pairs. Full data sets with standard deviation and reference heat maps showing fluorescence values resulting from c2 being exposed to corresponding doses of synthetic p2 can be found in FIG. 20.

FIG. 18C provides a schematic of an exemplary overview of implemented communication topologies. Grey nodes indicate yeast able to process one input (expressing one GPCR) and giving one output (secreting one peptide). Blue nodes indicate yeast cells able to process two inputs (OR gates, expressing two GPCRs) and giving one output (secreting one peptide). Red nodes indicate yeast cells able to receive a signal and respond by producing a fluorescent read-out.

FIG. 18D provides a graph reporting exemplary fluorescence readouts of fold-change in fluorescence between the full-ring and the interrupted ring indicated for each topology shown in FIG. 18C. Ring topologies with an increasing number of members (two to six) were established. The red nodes shown in FIG. 18C start and close the information flow through the ring by constitutively expressing the peptide for the next clockwise neighbor (starting) as well as they produce a fluorescent read-out upon receiving a peptide-signal from the counter-clockwise neighbor (closing). An interrupted ring, with one member dropped out, was used as the control. Fluorescence values were normalized by OD₆₀₀. Measurements were performed in triplicate and error bars represent the standard deviation.

FIG. 18E provides a graph reporting results of an exemplary three-yeast bus topology implemented as diagramed in FIG. 18C. The first yeast node can sense two inputs (OR gate) and the last node reports on functional information flow by producing a fluorescent read-out upon input sensing. Fluorescence values were normalized by OD₆₀₀. Measurements were performed in triplicate and error bars represent the standard deviation. Fluorescence was measured after induction with all possible combinations of the three input peptides (zero, one, two, or three peptides). The numbers above the bars indicate the fold-change in fluorescence over the no-peptide induction value.

FIG. 18F is a graph reporting results of an exemplary six-yeast branched tree-topology implemented as diagramed in FIG. 18C. The first yeast node can sense two inputs (OR gate) and the last node reports on functional information flow by producing a fluorescent read-out upon input sensing. Fluorescence values were normalized by OD₆₀₀. Measurements were performed in triplicate and error bars represent the standard deviation. Fluorescence was measured after induction with all possible combinations of the three input peptides (zero, one, two, or three peptides). The numbers above the bars indicate the fold-change in fluorescence over the no-peptide induction value.

FIGS. 19A-19H provide graphs reporting the full data set including error bars for the exemplary graphs shown in FIG. 18B. Transfer function strains were co-cultured in a 96-well plate (200 μl total culturing volume) with the appropriate fluorescent reporter strain and experiments were run in triplicate. The transfer function strain was induced with synthetic peptide at the following concentrations: 0 μM (H₂O blank), 0.0025 μM, 0.05 μM, 1.0 μM. The black curve for each GPCR represents a control in which the reporter strain was co-cultured with a non-GPCR strain (to maintain the 1:1 strain ratio) and directly induced with the same concentrations of the synthetic peptide.

FIG. 20 provides a schematic showing exemplary results for a control experiment for the exemplary data shown reported in FIG. 18B. Reference heat maps showing fluorescence values resulting from c2 being exposed to the indicated doses of synthetic p2.

FIG. 21 provides a schematic of an exemplary scalable communication ring topology. c1 serves as ring start and closing node. Signaling is started by c1 secreting p1 constitutively. Measuring fluorescence read-out in c1 allows the assessment of functional signal transmission through the ring.

FIG. 22 provides a summary of the exemplary strains used to create the two-to six-yeast paracrine communication rings (FIG. 18D). The first linker yeast strain (dropout) was removed to serve as a control for complete signal propagation through the communication ring.

FIG. 23 provides a graph reporting growth curves of exemplary communication strains Each strain was seeded in triplicate at OD=0.15 in 200 μL in a 96-well plate and measuring OD₆₀₀ values over 24 hours.

FIG. 24 provides a graph and table reporting exemplary results of colony PCR performed to confirm the presence of co-cultured strains. Samples were taken from a representative three-yeast communication loop and dropout control and plated to get single colonies on selective SD plates. Colony PCR was performed on 24 colonies from each time-point, running three separate PCR reactions in parallel, one for each strain using the integrated GPCR sequence as the strain-specific tag. The three separate PCR reactions were then pooled and visualized on a gel, and bands were counted to determine the ratios of the three communication strains. OD₆₀₀ and red fluorescence measurements were taken in triplicate and processed as for the multi-yeast communication loops.

FIG. 25 provides a schematic of an exemplary 6-yeast branched tree-topology (Topology 8, FIG. 18C). c1, c2 and c5 are induced with synthetic peptides p1, p2 and p3 to start communication. FIG. 18F features induction with each single peptide, all combinations of two peptides or all three peptides. c6 serves as closing node. Measuring fluorescence read-out in c6 allows the assessment of functional signal transmission through the topology. Topology 6 of FIG. 18C involves cells c3, c4 and c6. Topology 7 of FIG. 18C involves cells c1, c2, c4, c5 and c6.

FIG. 26 is a summary of the exemplary strains used to create exemplary bus and branched tree topologies (FIGS. 18E and F).

FIG. 27A provides a schematic of exemplary interdependent microbial communities mediated by the peptide-based synthetic communication language. Peptide-signal interdependence was achieved by placing an essential gene (SEC4) under GPCR control. In the featured three-yeast ring c1, c2 and c3 secret the peptide needed for growth of the cx-1 member of the ring. Peptides are secreted from the constitutive ADH1 promoter.

FIG. 27B and FIG. 27C provide graphs reporting results of growth of an exemplary three-membered interdependent microbial community over >7 days. Communities with one essential member dropped out collapse after ˜two days (as shown in FIG. 27C). Three-membered communities were seeded in a 1:1:1 ratio, controls were seeded using the same cell numbers for each member as for the three-membered community. All experiments were run in triplicate and error bars represent the standard deviation.

FIG. 27D provides a graph reporting exemplary results of the composition of an exemplary culture tracked over time by taking samples from one of the triplicates at the indicated time points, plating the cells on media selective for each of the three component strains, and colony counting.

FIG. 28A provides schematics of structure and function of an exemplary Ste12*.

FIG. 28B provides a graph reporting exemplary dose response curves of Bc.Ste2 using a red fluorescent protein driven by OSR2 and OSR4 as read-out. The dotted blue line indicates the expected intracellular levels of Sec4. Levels were estimated by cloning the SEC4 promoter in front of a red fluorescent read-out and comparing fluorescent/OD values to the OSR promoter read-out.

FIG. 28C provides images of exemplary results of a dot assay of peptide dependent strains ySB268/270 (Ca peptide-dependent strains), ySB188 (Vp1 peptide-dependent strain) and ySB24/265 (Bc peptide-dependent strains) in the presence and absence of peptide. Serial 10-fold dilutions of overnight cultures were spotted on SD agar plates supplemented with or without 1 μM peptide and incubated at 30° C. for 48 hours. Strains ySB264 and ySB268 are individually isolated replicate colonies of strains ySB265 and ySB270.

FIGS. 29A-29C provides graphs reporting exemplary EC₅₀ of growth for peptide dependent strains. After several doublings the peptide-dependent strains ySB265 (Bc.Ste2) (FIG. 29A), ySB270 (Ca.Ste2) (FIG. 29B) and ySB188 (Vp1.Ste2) (FIG. 29C) show peptide-concentration dependent growth behavior. The final OD of this experiment (indicated by a dotted box in each panel) was used to calculate the EC₅₀ of growth for each strain: OD values were plotted against the log₁₀-converted peptide concentrations peptide concentration and the data were fit to a four-parameter non-linear regression model using Prism (GraphPad). Strains were cultured overnight in the presence of 100 nM peptide in SC(-His). Cells were washed five times with one volumes of water. Cells were than seeded in 200 μl SC (no selection) at an OD₆₀₀ of 0.06 and cultured at 30° C. and 800 RPM shaking. Cells were exposed to the indicated concentrations of peptide and OD₆₀₀ was determined at the indicated time points. After an initial 12-hour growth, cells were diluted 1:20 into fresh media. Growth was then followed over the course of an additional 24 hours.

FIG. 30 provides graphs reporting results and schematics of exemplary interdependent 2-Yeast links. Strains ySB265 (Bc.Ste2), ySB270 (Ca.Ste2) and ySB188 (Vp1.Ste2) were transformed with the appropriate peptide secretion vectors (Bc, Ca or Vp1) featuring peptide expression under the constitutive ADH1 promoter. The six resulting strains were used to assemble all three possible 2-Yeast combinations. The key to the peptide and GPCR combinations is given in the schematic shown to the right of graphs in Panels a-c. The resulting peptide-secreting strains were seeded in the appropriate combination in a 1:1 ratio in triplicate cultures. The same cell number of single strains was seeded alone and cultured in parallel as control. OD₆₀₀ measurements were taken at the indicated time points and cultures were diluted 1:20 into fresh media at the indicated time points. Co-cultured were maintained for 67 hours.

FIG. 31 provides graphs reporting results of peptide concentrations in exemplary 3-Yeast ecosystem. The peptide concentration in each sample (sample number corresponds to FIG. 5F) was determined by using the corresponding GPCR/Fluorescent read-out strain (JTy014 expressing Bc, Ca or Vp1.Ste2). Panel a: Ca peptide; Panel b: Bc peptide; Panel c: Vp1 peptide. The linear range of the dose response curve of each GPCR was used for peptide quantification. The Ca peptide was not precisely quantified as several fluorescent values were out of the linear range; therefore, the Y-axis of panel a therefore gives approximate amounts.

### DETAILED DESCRIPTION

The present disclosure relates to the use of G-protein coupled receptor (GPCR)-ligand pairs to promote intercellular signaling between genetically-engineered cells. For example, but not by way of limitation, the present disclosure provides intercellular signaling systems that include two or more genetically-engineered cells that communicate with each other, and kits thereof. In particular, the scalable GPCR-peptide intercellular signaling system described herein is generally useful for engineering multicellular systems based on unicellular organisms, e.g., yeast.

For clarity, but not by way of limitation, the detailed description of the presently disclosed subject matter is divided into the following subsections:

I. Definitions;

II. G protein-coupled receptors (GPCRs) and cognate ligands;

III. Cells;

IV. Intracellular signaling networks;

V. Methods of Use;

VI. Kits; and

VII. Exemplary Embodiments.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “expression” or “expresses,” as used herein, refer to transcription and translation occurring within a cell, e.g., yeast cell. The level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, “polypeptide” refers generally to peptides and proteins having about three or more amino acids. In certain embodiments, the polypeptide comprises the minimal amount of amino acids that are detectable by a G-protein coupled receptor (GPCR). The polypeptides can be endogenous to the cell, or preferably, can be exogenous, meaning that they are heterologous, i.e., foreign, to the cell being utilized, such as a synthetic peptide and/or GPCR produced by a yeast cell. In certain embodiments, synthetic peptides are used, more preferably those which are directly secreted into the medium.

The term “protein” is meant to refer to a sequence of amino acids for which the chain length is sufficient to produce the higher levels of tertiary and/or quaternary structure. This is to distinguish from “peptides” that typically do not have such structure. Typically, the protein herein will have a molecular weight of at least about 15-100 kD, e.g., closer to about 15 kD. In certain embodiments, a protein can include at least about 50, about 60, about 70, about 80, about 90, about 100, about 200, about 300, about 400 or about 500 amino acids. Examples of proteins encompassed within the definition herein include all proteins, and, in general proteins that contain one or more disulfide bonds, including multi-chain polypeptides comprising one or more inter- and/or intrachain disulfide bonds. In certain embodiments, proteins can include other post-translation modifications including, but not limited to, glycosylation and lipidation. See, e.g., Prabakaran et al., WIREs Syst Biol Med (2012), which is incorporated herein by reference in its entirety.

As used herein the term “amino acid,” “amino acid monomer” or “amino acid residue” refers to organic compounds composed of amine and carboxylic acid functional groups, along with a side-chain specific to each amino acid. In particular, alpha- or α-amino acid refers to organic compounds in which the amine (—NH2) is separated from the carboxylic acid (—COOH) by a methylene group (—CH2), and a side-chain specific to each amino acid connected to this methylene group (—CH2) which is alpha to the carboxylic acid (—COOH). Different amino acids have different side chains and have distinctive characteristics, such as charge, polarity, aromaticity, reduction potential, hydrophobicity, and pKa. Amino acids can be covalently linked to form a polymer through peptide bonds by reactions between the carboxylic acid group of the first amino acid and the amine group of the second amino acid. Amino acid in the sense of the disclosure refers to any of the twenty plus naturally occurring amino acids, non-natural amino acids, and includes both D and L optical isomers.

The term “nucleic acid,” “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an GPCR or secretable peptide of the disclosure in vitro and/or in vivo, e.g., in a yeast cell. Such DNA (e.g., cDNA) or RNA (e.g., mRNA) vectors, can be unmodified or modified. For example, mRNA can be chemically modified to enhance the stability of the RNA vector and/or expression of the encoded molecule.

As used herein, the term “vector” refers to a nucleic acid molecule capable of transporting another nucleic acid to which it has been linked.

As used herein, the term “recombinant cell” refers to cells which have some genetic modification from the original parent cells from which they are derived. Such cells can also be referred to as “genetically-engineered cells.” Such genetic modification can be the result of an introduction of a heterologous gene (or nucleic acid) for expression of the gene product, e.g., a recombinant protein, e.g., GPCR, or peptide, e.g., secretable peptide.

As used herein, the term “recombinant protein” refers generally to peptides and proteins. Such recombinant proteins are “heterologous,” i.e., foreign to the cell being utilized, such as a heterologous secretory peptide produced by a yeast cell.

As used herein, “sequence identity” or “identity” in the context of two polynucleotide or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

As used herein, “percentage of sequence identity” means the value determined by comparing two optimally aligned sequences over a comparison window, wherein the portion of the polynucleotide sequence in the comparison window can include additions or deletions (gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As understood by those skilled in the art, determination of percent identity between any two sequences can be accomplished using certain well-known mathematical algorithms. Non-limiting examples of such mathematical algorithms are the algorithm of Myers and Miller, the local homology algorithm of Smith et al.; the homology alignment algorithm of Needleman and Wunsch; the search-for-similarity-method of Pearson and Lipman; the algorithm of Karlin and Altschul, modified as in Karlin and Altschul. Computer implementations of suitable mathematical algorithms can be utilized for comparison of sequences to determine sequence identity. Such implementations include, but are not limited to: CLUSTAL, ALIGN, GAP, BESTFIT, BLAST, FASTA, among others identifiable by skilled persons.

As used herein, “reference sequence” is a defined sequence used as a basis for sequence comparison. A reference sequence can be a subset or the entirety of a specified sequence; for example, as a segment of a full-length protein or protein fragment. A reference sequence can be, for example, a sequence identifiable in a database such as GenBank and UniProt and others identifiable to those skilled in the art.

The term “operative connection” or “operatively linked,” as used herein, with regard to regulatory sequences of a gene indicate an arrangement of elements in a combination enabling production of an appropriate effect. With respect to genes and regulatory sequences, an operative connection indicates a configuration of the genes with respect to the regulatory sequence allowing the regulatory sequences to directly or indirectly increase or decrease transcription or translation of the genes. In particular, in certain embodiments, regulatory sequences directly increasing transcription of the operatively linked gene, comprise promoters typically located on a same strand and upstream on a DNA sequence (towards the 5′ region of the sense strand), adjacent to the transcription start site of the genes whose transcription they initiate. In certain embodiments, regulatory sequences directly increasing transcription of the operatively linked gene or gene cluster comprise enhancers that can be located more distally from the transcription start site compared to promoters, and either upstream or downstream from the regulated genes, as understood by those skilled in the art. Enhancers are typically short (50-1500 bp) regions of DNA that can be bound by transcriptional activators to increase transcription of a particular gene. Typically, enhancers can be located up to 1 Mbp away from the gene, upstream or downstream from the start site.

The term “secretable,” as used herein, means able to be secreted, wherein secretion in the present disclosure generally refers to transport or translocation from the interior of a cell, e.g., within the cytoplasm or cytosol of a cell, to its exterior, e.g., outside the plasma membrane of the cell. Secretion can include several procedures, including various cellular processing procedures such as enzymatic processing of the peptide. In certain embodiments, secretion, e.g., secretion of a GPCR ligand, can utilize the classical secretory pathway of yeast.

As would be understood by those skilled in the art, the term “codon optimization,” as used herein, refers to the introduction of synonymous mutations into codons of a protein-coding gene in order to improve protein expression in expression systems of a particular organism, such as a cell of a species of the phylum Ascomycota, in accordance with the codon usage bias of that organism. The term “codon usage bias” refers to differences in the frequency of occurrence of synonymous codons in coding DNA. The genetic codes of different organisms are often biased towards using one of the several codons that encode a same amino acid over others—thus using the one codon with, a greater frequency than expected by chance. Optimized codons in microorganisms, such as Saccharomyces cerevisiae, reflect the composition of their respective genomic tRNA pool. The use of optimized codons can help to achieve faster translation rates and high accuracy.

In the field of bioinformatics and computational biology, many statistical methods have been discussed and used to analyze codon usage bias. Methods such as the ‘frequency of optimal codons’ (Fop), the Relative Codon Adaptation (RCA) or the ‘Codon Adaptation Index’ (CAI) are used to predict gene expression levels, while methods such as the ‘effective number of codons’ (Nc) and Shannon entropy from information theory are used to measure codon usage evenness. Multivariate statistical methods, such as correspondence analysis and principal component analysis, are widely used to analyze variations in codon usage among genes. There are many computer programs to implement the statistical analyses enumerated above, including CodonW, GCUA, INCA, and others identifiable by those skilled in the art. Several software packages are available online for codon optimization of gene sequences, including those offered by companies such as GenScript, EnCor Biotechnology, Integrated DNA Technologies, ThermoFisher Scientific, among others known those skilled in the art. Those packages can be used in providing GPCR genetic molecular components and GPCR peptide ligand genetic molecular components with codon ensuring optimized expression in various intercellular signaling systems as will be understood by a skilled person.

The term “binding,” as used herein, refers to the connecting or uniting of two or more components by a interaction, bond, link, force or tie in order to keep two or more components together, which encompasses either direct or indirect binding where, for example, a first component is directly bound to a second component, or one or more intermediate molecules are disposed between the first component and the second component. Exemplary bonds comprise covalent bond, ionic bond, van der Waals interactions and other bonds identifiable by a skilled person. In certain embodiments, the binding can be direct, such as the production of a polypeptide scaffold that directly binds to a scaffold-binding element of a protein. In certain embodiments, the binding can be indirect, such as the co-localization of multiple protein elements on one scaffold. In certain embodiments, binding of a component with another component can result in sequestering the component, thus providing a type of inhibition of the component. In certain embodiments, binding of a component with another component can change the activity or function of the component, as in the case of allosteric or other interactions between proteins that result in conformational change of a component, thus providing a type of activation of the bound component. Examples described herein include, without limitation, binding of a GPCR ligand, e.g., peptide ligand, to a GPCR.

The term “selectively activates,” as used herein, refers to the ability of a ligand, e.g., peptide, to activate a receptor, e.g., preferentially interact with, in the presence of other different receptors. In certain embodiments, a ligand can selectively activate two different GPCRs in the presence of other receptors.

The term “reportable component,” as used herein, indicates a component capable of detection in one or more systems and/or environments.

The terms “detect” or “detection,” as used herein, indicates the determination of the existence and/or presence of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

The term “derived” or “derive” is used herein to mean to obtain from a specified source.

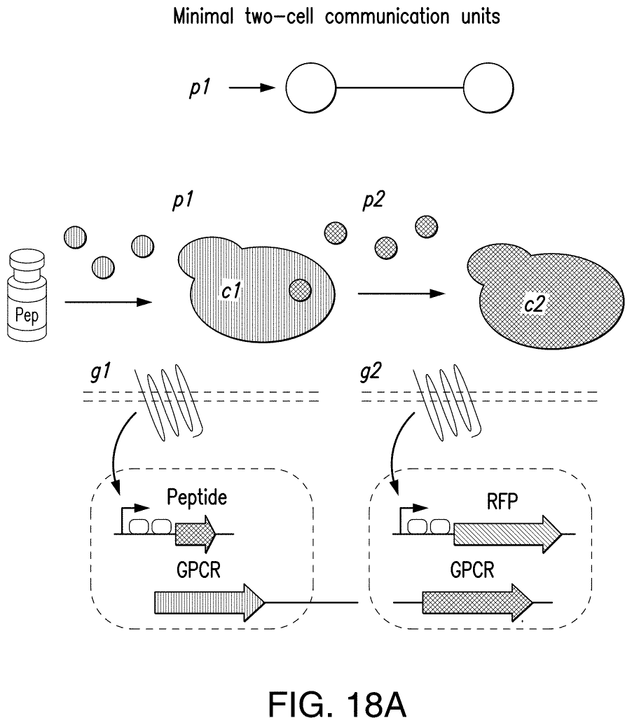

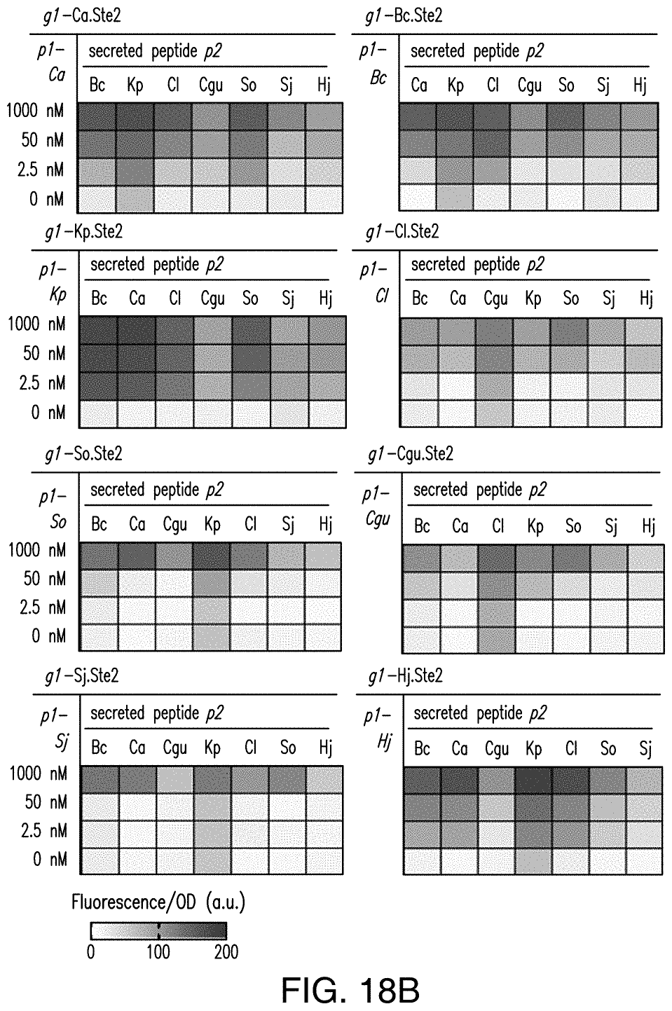

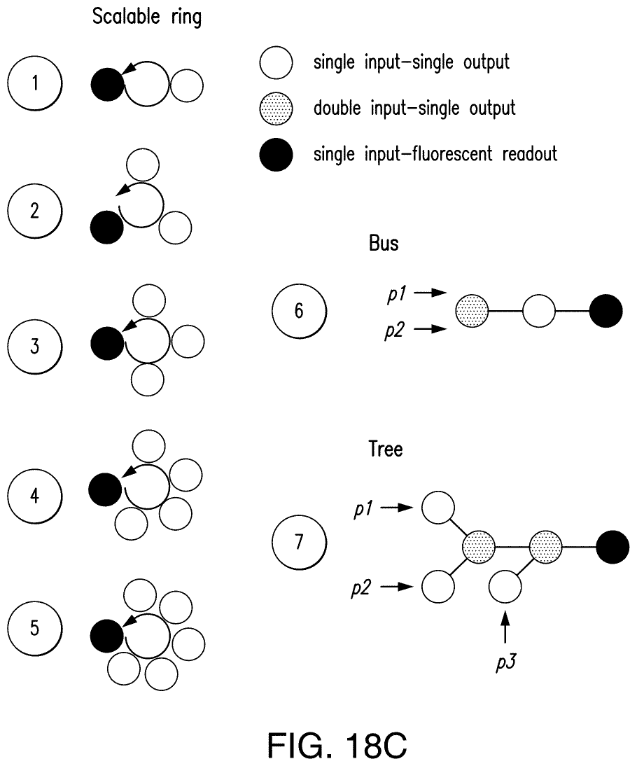

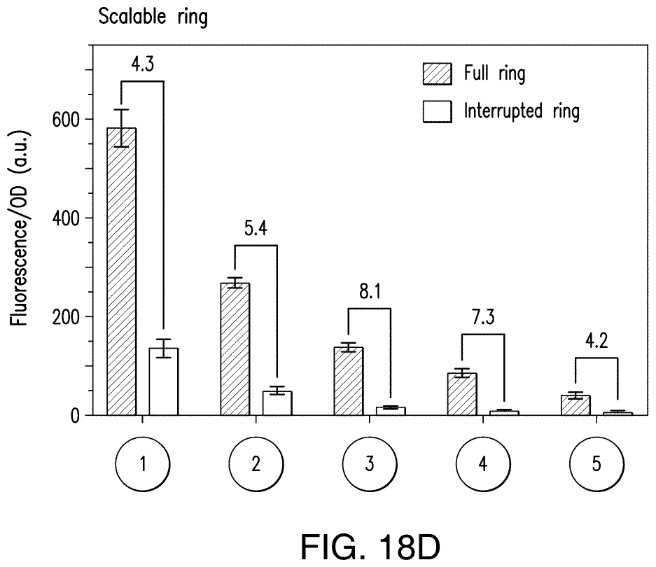

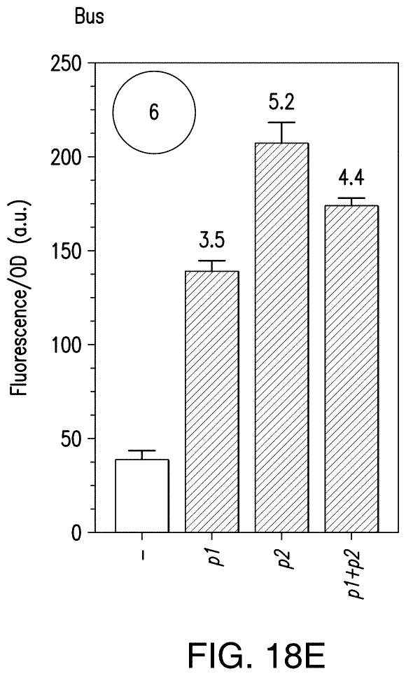

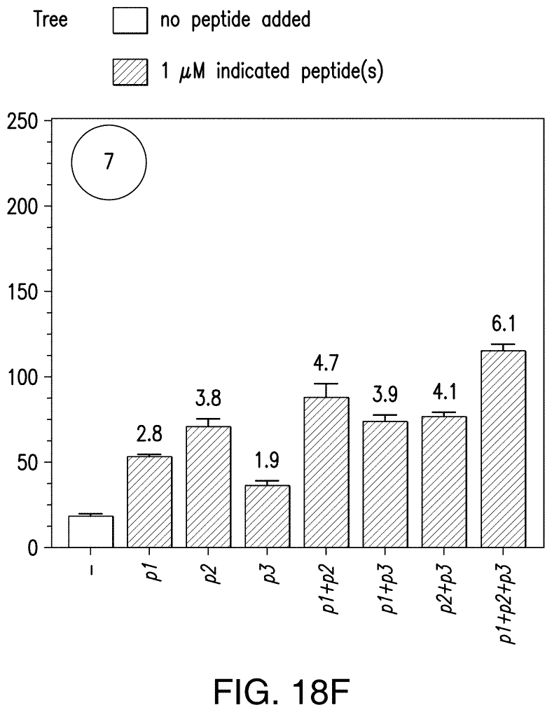

The term “daisy-chaining,” as used herein, refers to a method of providing a network having greater complexity than a point-to-point network, wherein adding more nodes (e.g., more than two linked cells) is achieved by linking each additional node (e.g., cell) one to another. Accordingly, in a “daisy chain” type of network comprising multiple nodes (e.g., multiple different types of cells), a signal is passed through the network from one node (e.g., cell) to another in series in a stepwise manner, from a first terminal node (e.g., cell) to a second terminal node (e.g., cell) through one or more intermediary nodes (e.g., cells). This can be contrasted, for example, to a “bus” type of network wherein nodes can be connected to each other through a singular common link. A “daisy chain” network topology can be a daisy chain linear network topology or a daisy chain ring network topology. In certain embodiments, a daisy chain linear network topology or a daisy chain ring network topology can further comprise one or more branches that extend from one or more intermediary nodes (e.g., cells) in the network topology, also referred to herein as a “branched” network topology. In certain embodiments, the “branched” network has a “star” topology or a “ring” topology. Non-limiting examples of daisy chain network configurations are shown in FIGS. 18A, 18C, 21, 25 and 27A. In certain embodiments, an intercellular signaling system of the present disclosure can have a combination of two or more topologies, i.e., a “hybrid” topology. In certain embodiments, an intercellular signaling system of the present disclosure can have a “mesh” topology.

A “star” network topology, as used herein, refers to a network that includes branches, e.g., a cell or cells, that can be connected to each other through a singular common link, e.g., cell.

A “mesh” network topology, as used herein, refers to a network where all the cells with the network are connected to as many other cells as possible.

A “ring” network topology, as used herein, refers to a network that comprises cells that are connected in a manner where the last cell in the chain is connected back to the first cell in the chain. Non-limiting examples of ring network configurations are shown in FIGS. 18C, 21 and 27A.

A “bus” type of network topology, as used herein, and as referenced above, can refer to a network of cells comprising cells that can be connected to each other through a singular common cell. A non-limiting example of a bus type of network is shown in FIG. 18C.

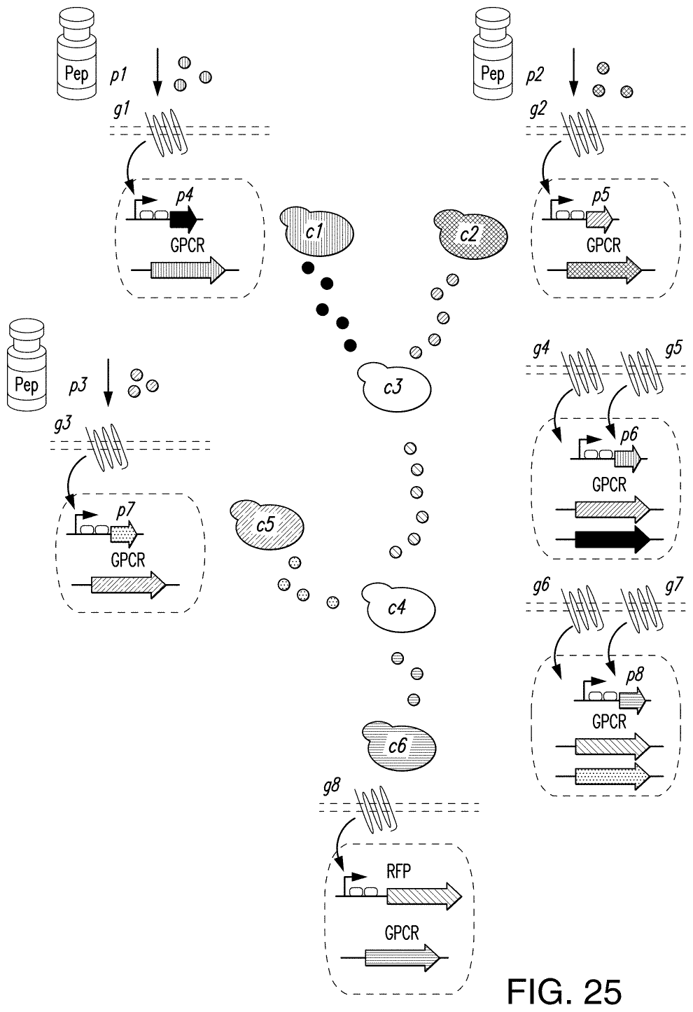

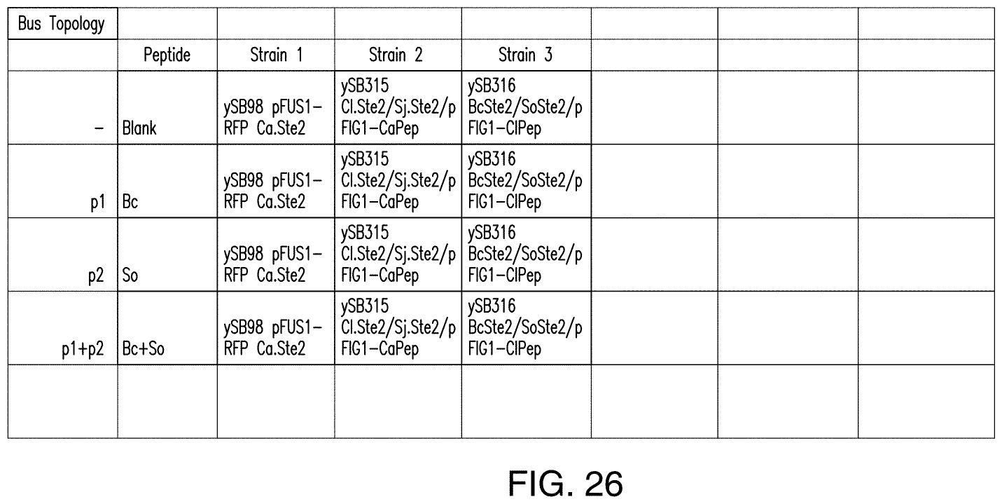

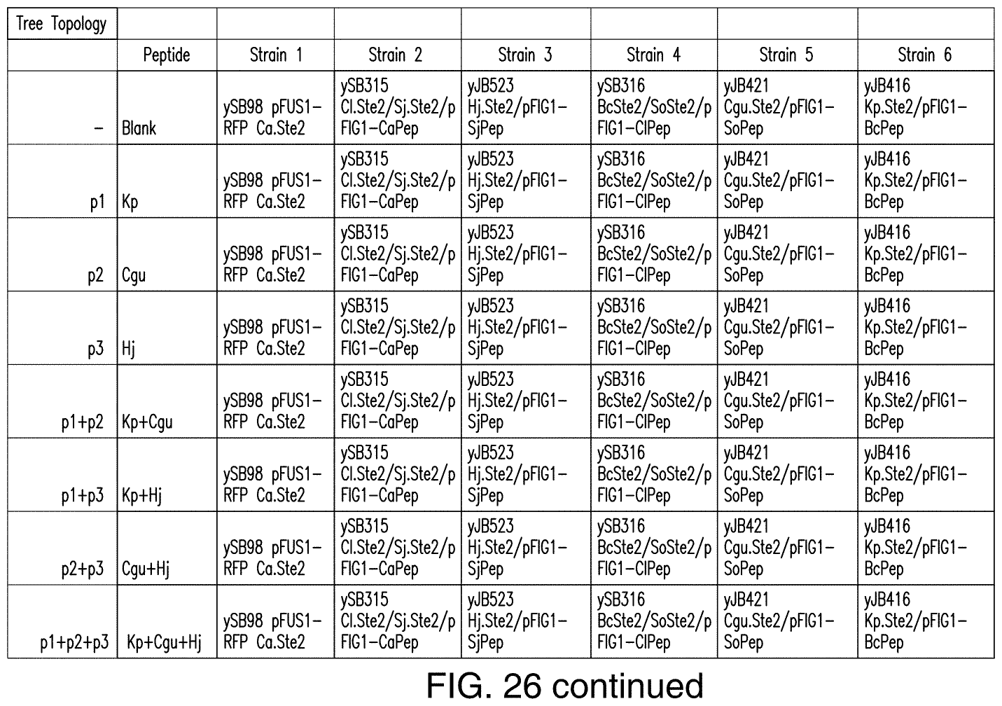

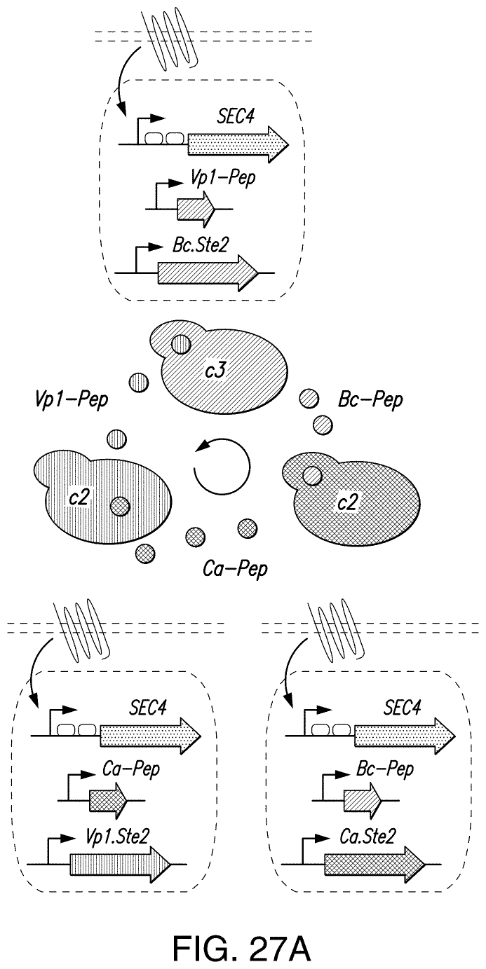

A “branched” type of network topology, as used herein, and as referenced above, can refer to a network of cells that include one or more branches that extend from one or more intermediary cells. Non-limiting examples of branched type network configurations are shown in FIGS. 18C and 25.

### II. G Protein-Coupled Receptors (GPCRs) and Cognate Ligands

The present disclosure provides GPCRs and ligands for an intercellular communication language between two or more cells, e.g., of the phylum Ascomycota. In certain embodiments, the intercellular signaling system utilizes expression vectors to achieve expression of GPCRs and cognate ligands in fungal cells, e.g., yeast cells (e.g., S. cerevisiae).

GPCRs

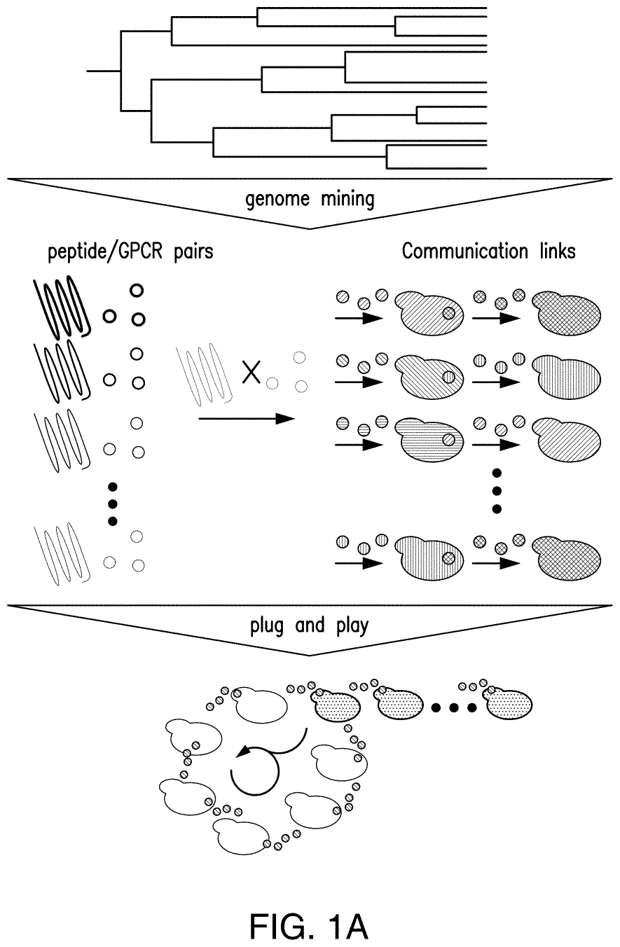

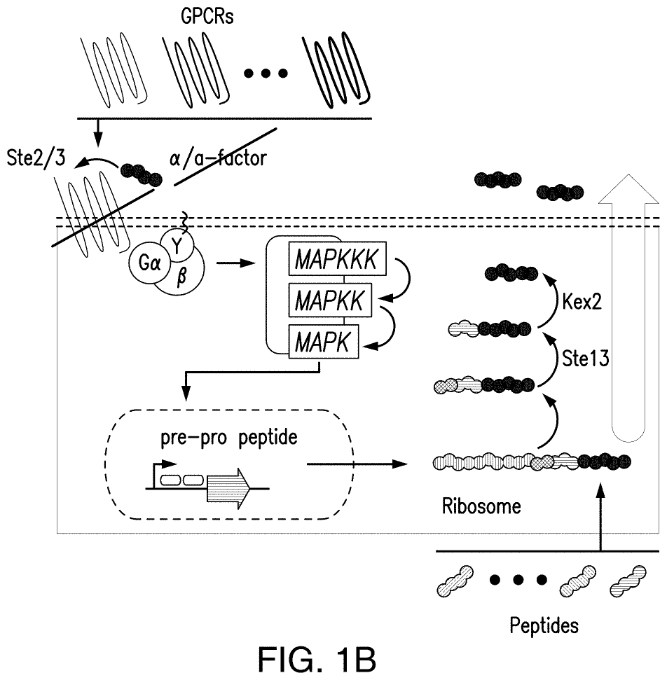

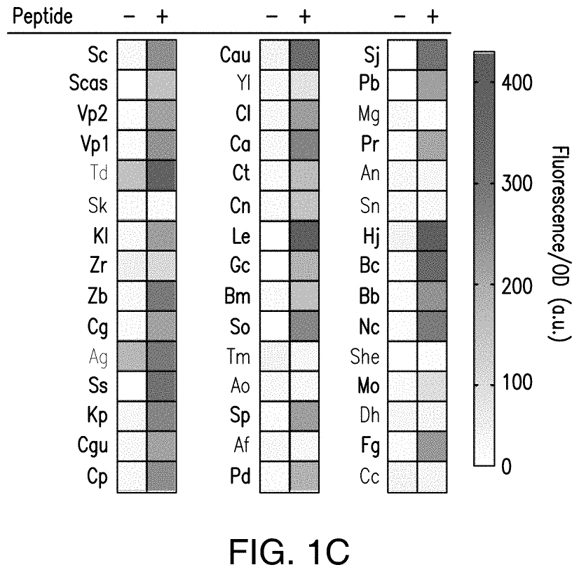

G protein-coupled receptors (GPCRs), also known as seven-transmembrane domain receptors, 7TM receptors, heptahelical receptors, serpentine receptor and G protein-linked receptors (GPLR), constitute a large protein family of receptors that detect molecules outside the cell and activate internal signal transduction pathways and, ultimately, cellular responses. G protein-coupled receptors are found only in eukaryotes, such as yeast and animals. The ligands that bind and activate these receptors include light-sensitive compounds, odors, pheromones, hormones, toxins, and neurotransmitters, and vary in size from small molecules to peptides to large proteins. When a ligand binds to the GPCR it causes a conformational change in the GPCR, allowing it to act as a guanine nucleotide exchange factor (GEF). The GPCR can then activate an associated G protein by exchanging the GDP bound to the G protein for a GTP. The G protein's a subunit, together with the bound GTP, can then dissociate from the β and γ subunits to further affect intracellular signaling proteins or target functional proteins directly depending on the a subunit type (Gαs, Gαi/o, Gαq/11, Gα12/13) (see, e.g., FIG. 1A).

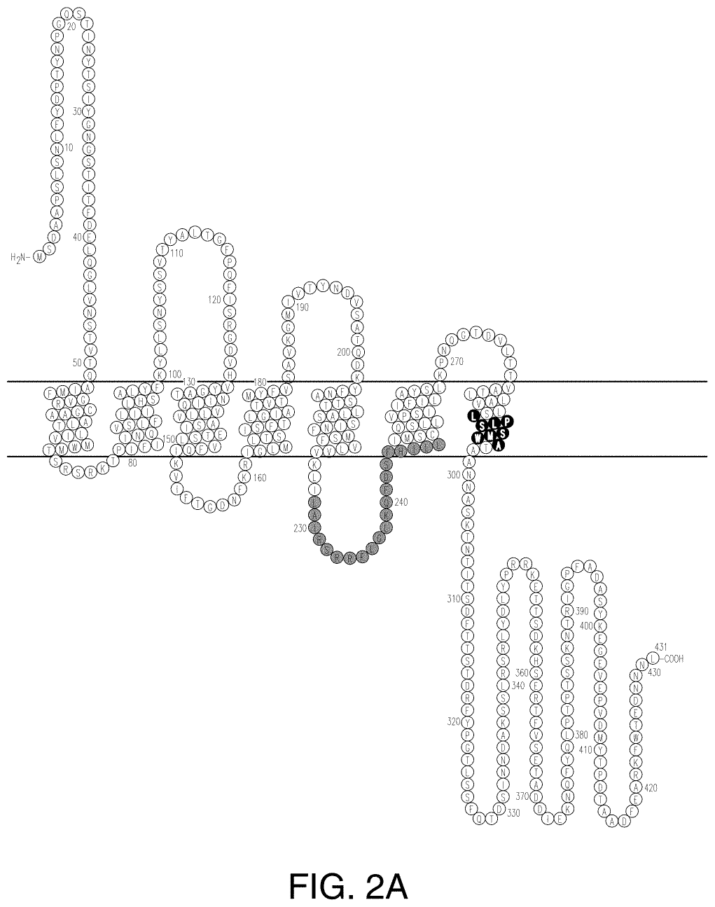

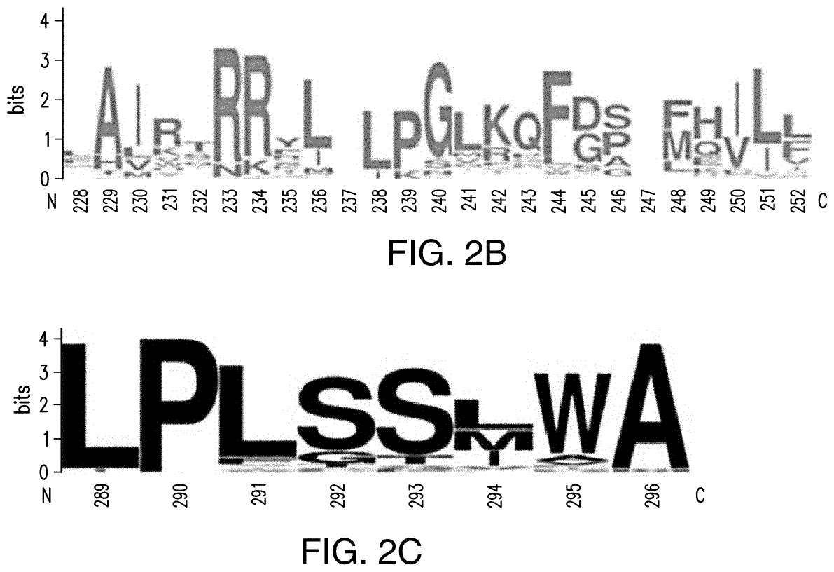

The present disclosure provides GPCRs for use in the intercellular signaling systems of the present disclosure. In certain embodiments, the GPCRs for use in the present disclosure can be identified and/or derived from any eukaryotic organism, e.g., an animal, plant, fungus and/or protozoan. In certain embodiments, GPCRs for use in the present disclosure can be identified and/or derived from mammalian cells. In certain embodiments, GPCRs for use in the present disclosure can be identified and/or derived from plant cells. In certain embodiments, GPCRs for use in the present disclosure can be identified and/or derived from fungal cells, e.g., a fungal GPCR. For example, but not by way of limitation, GPCRs for use in the present disclosure can be identified and/or derived from Metozoans, Unicellular Holozoa and Amoebazoa. Additional non-limiting examples of organisms that can be used to identify and/or derive GPCRs for use in the present disclosure is provided in FIG. 2 of Mendoza et al., Genome Biol. Evol. 6(3):606-619 (2014), which is incorporated herein in its entirety.

In certain embodiments, a GPCR of the present disclosure can be identified and/or derived from the genome of a species of the phylum Ascomycota. Ascomycota is a division or phylum of the kingdom Fungi that, together with the Basidiomycota, form the subkingdom Dikarya. Its members are commonly known as the sac fungi or ascomycetes. Ascomycota is the largest phylum of Fungi, with over 64,000 species. A defining feature of this fungal group is the ascus, a microscopic sexual structure in which nonmotile spores, called ascospores, are formed. Ascomycetes can be identified and classified based on morphological or physiological similarities, and by phylogenetic analyses of DNA sequences (e.g., as described in Lutzoni F. et al. (2004), American Journal of Botany 91 (10): 1446-80 and James TY. et al. (2006), Nature 443 (7113): 818-22). Non-limiting examples of such species include Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, and Capronia coronate. See also Table 3, which provides a list of potential species from which GPCRs can be obtained and/or derived. In certain embodiments, the GPCR is identified and/or derived from the genome of Saccharomyces cerevisiae.

In certain embodiments, the GPCR or portion thereof for use in the present disclosure is a seven-transmembrane domain receptor that can be selectively activated by interaction with a ligand. In certain embodiments, the GPCR or portion thereof for use in the present disclosure can interact with and activate G proteins.

In certain embodiments, the GPCR or a portion thereof for use in the present disclosure comprises an amino acid sequence of any one of SEQ ID NOs: 117-161, or conservative substitutions thereof or a homolog thereof (see Table 9). In certain embodiments, the GPCR or portion thereof comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence comprising any one of SEQ ID NOs: 117-161.

In certain embodiments, the GPCR or a portion thereof for use in the present disclosure comprises a nucleotide sequence of any of SEQ ID NOs: 168-211, or conservative substitutions thereof or a homolog thereof (see Table 5). In certain embodiments, the GPCR or portion thereof comprises a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence comprising any one of SEQ ID NOs: 168-211.

In certain embodiments, the GPCR or a portion thereof for use in the present disclosure comprises an amino acid sequence of any one of the GPCRs disclosed in Table 4 and Table 6 of U.S. Publication No. 2017/0336407, the content of which is incorporated in its entirety by reference herein. For example, but not by way of limitation, the GPCR or portion thereof comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence disclosed in Table 4 and Table 6 of U.S. Publication No. 2017/0336407.

In certain embodiments, the GPCR or a portion thereof for use in the present disclosure comprises an amino acid sequence of any one of the GPCRs listed in Table 11. In certain embodiments, the GPCR or portion thereof comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of any one of the GPCRs listed in Table 11.

TABLE 11
Non-Limiting Embodiments of GPCRS

| | Receptor | Species | | |
|---|---|---|---|---|
| Species name | UniProt ID | Tax. ID | Family | Order |
| Acidomyces richmondensis BFW | A0A150VDK8 | 766039 | Dothideomycetes | Dothideomycetes |
| | | | incertae sedis | |
| Acremonium_chrysogenum_strain_ATCC 11550 | A0A086SWK6 | 857340 | Hypocreales incertae | Hypocreales |
| | | | sedis | |
| Ajellomyces capsulatus strain G186AR | C0NQ16 | 447093 | Ajellomycetaceae | Onygenales |
| Ajellomyces_capsulatus_strain_H143 | C6HLQ1 | 544712 | Ajellomycetaceae | Onygenales |
| Ajellomyces_capsulatus_strain_NAm1 | A6QUU6 | 339724 | Ajellomycetaceae | Onygenales |
| Ajellomyces_dermatitidis_strain_SLH14081 | A0A179UUK7 | 559298 | Ajellomycetaceae | Onygenales |
| Alternaria alternata | A0A177DMP1 | 5599 | Pleosporaceae | Pleosporales |
| Arthrobotrys_oligospora_strain_ATCC_24927 | G1X8M4 | 756982 | Orbiliaceae | Orbiliales |
| Arthroderma_benhamiae_strain_ATCC_MYA-4681 | D4AND1 | 663331 | Arthrodermataceae | Onygenales |
| Arthroderma_gypseum_strain_ATCC_MYA-4604 | E5R1C9 | 535722 | Arthrodermataceae | Onygenales |
| Arthroderma_otae_strain_ATCC_MYA-4605 | C5FBT2 | 554155 | Arthrodermataceae | Onygenales |
| Aschersonia aleyrodis RCEF 2490 | A0A168AUR9 | 1081109 | Clavicipitaceae | Hypocreales |
| Ascosphaera apis ARSEF 7405 | A0A167VMP9 | 392613 | Ascosphaeraceae | Onygenales |
| Ashbya_aceri | R9XEV1 | 566037 | Saccharomycetaceae | Saccharomycetales |
| Ashbya_gossypii_strain_ATCC_10895 | Q752Q1 | 284811 | Saccharomycetaceae | Saccharomycetales |
| Aspergillus calidoustus | A0A0U5CD47 | 454130 | Aspergillaceae | Eurotiales |
| Aspergillus clavatus strain ATCC 1007 | A1CLD3 | 344612 | Aspergillaceae | Eurotiales |
| Aspergillus flavus strain ATCC 200026 | B8NF30 | 332952 | Aspergillaceae | Eurotiales |
| Aspergillus_fumigatus_Z5 | A0A0J5PTK8 | 1437362 | Aspergillaceae | Eurotiales |
| Aspergillus_kawachii_strain_NBRC_4308 | G7XMN4 | 1033177 | Aspergillaceae | Eurotiales |
| Aspergillus lentulus | A0A0S7DJF6 | 293939 | Aspergillaceae | Eurotiales |
| Aspergillus luchuensis | A0A146FQ34 | 1069201 | Aspergillaceae | Eurotiales |
| Aspergillus niger | A0A100IM28 | 5061 | Aspergillaceae | Eurotiales |
| Aspergillus niger strain CBS 51388 | A2QU32 | 425011 | Aspergillaceae | Eurotiales |
| Aspergillus nomius NRRL 13137 | A0A0L1J1T8 | 1509407 | Aspergillaceae | Eurotiales |
| Aspergillus ochraceoroseus | A0A0F8U8N5 | 138278 | Aspergillaceae | Eurotiales |
| Aspergillus_oryzae_strain_3042 | I8U4V3 | 1160506 | Aspergillaceae | Eurotiales |
| Aspergillus_parasiticus_strain_ATCC_56775 | A0A0F0I7R7 | 1403190 | Aspergillaceae | Eurotiales |
| Aspergillus rambellii | A0A0F8U3T7 | 308745 | Aspergillaceae | Eurotiales |
| Aspergillus ruber CBS 135680 | A0A017S298 | 1388766 | Aspergillaceae | Eurotiales |
| Aspergillus terreus strain NIH 2624 | Q0CS34 | 341663 | Aspergillaceae | Eurotiales |
| Aspergillus_udagawae | A0A0K8L9B1 | 91492 | Aspergillaceae | Eurotiales |
| Aureobasidium_melanogenum_CBS_110374 | A0A074VLE7 | 1043003 | Aureobasidiaceae | Dothideales |
| Aureobasidium namibiae CBS 14797 | A0A074XMD1 | 1043004 | Aureobasidiaceae | Dothideales |
| Aureobasidium pullulans EXF-150 | A0A074XT98 | 1043002 | Aureobasidiaceae | Dothideales |
| Aureobasidium subglaciale EXF-2481 | A0A074YTM0 | 1043005 | Aureobasidiaceae | Dothideales |
| Baudoinia_compniacensis_strain_UAMH_10762 | M2LX19 | 717646 | Teratosphaeriaceae | Capnodiales |
| Beauveria_bassiana_D1-5 | A0A0A2VS91 | 1245745 | Cordycipitaceae | Hypocreales |
| Beauveria bassiana strain ARSEF 2860 | J5JMP7 | 655819 | Cordycipitaceae | Hypocreales |
| Bionectria ochroleuca | A0A0B7KEZ6 | 29856 | Bionectriaceae | Hypocreales |
| Bipolaris oryzae ATCC 44560 | W6Z6J4 | 930090 | Pleosporaceae | Pleosporineae |
| Bipolaris_victoriae_FI3 | W7EF59 | 930091 | Pleosporaceae | Pleosporineae |
| Bipolaris_zeicola_26-R-13 | W6YNK7 | 930089 | Pleosporaceae | Pleosporineae |
| Blastobotrys adeninivorans | A0A060T2K3 | 409370 | Trichomonascaceae | Saccharomycetales |
| Blumeria_graminis_f_sp_hordei_strain_DH14 | N1J7M2 | 546991 | Erysiphaceae | Erysiphales |
| Botryosphaeria parva strain UCR-NP2 | R1GET9 | 1287680 | Botryosphaeriaceae | Botryosphaeriales |
| Botryotinia fuckeliana strain T4 | G2YE05 | 999810 | Sclerotiniaceae | Helotiales |
| Byssochlamys spectabilis strain No 5 | V5GA62 | 1356009 | Thermoascaceae | Eurotiales |
| Candida albicans P75010 | A0A0A6JZS6 | 1094994 | Debaryomycetaceae | Saccharomycetales |
| Candida_albicans_strain_SC5314 | Q59Q04 | 237561 | Debaryomycetaceae | Saccharomycetales |
| Candida_albicans_strain_WO-1 | C4YM83 | 294748 | Debaryomycetaceae | Saccharomycetales |
| Candida auris | A0A0L0P8C9 | 498019 | Metschnikowiaceae | Saccharomycetales |
| Candida dubliniensis strain CD36 | B9WM67 | 573826 | Debaryomycetaceae | Saccharomycetales |
| Candida glabrata | A0A0W0DD93 | 5478 | Saccharomycetaceae | Saccharomycetales |
| Candida_glabrata_strain_ATCC_2001 | Q6FLY8 | 284593 | Saccharomycetaceae | Saccharomycetales |
| Candida_maltosa_strain_Xu316 | M3K0H9 | 1245528 | Debaryomycetaceae | Saccharomycetales |
| Candida orthopsilosis strain 90-125 | H8X566 | 1136231 | Debaryomycetaceae | Saccharomycetales |
| Candida parapsilosis strain CDC 317 | G8BFM9 | 578454 | Debaryomycetaceae | Saccharomycetales |
| Candida tenuis strain ATCC 10573 | G3BD19 | 590646 | Debaryomycetaceae | Saccharomycetales |
| Candida_tropicalis_strain_ATCC_MYA-3404 | C5M3P6 | 294747 | Debaryomycetaceae | Saccharomycetales |
| Capronia_epimyces_CBS_60696 | W9X9V4 | 1182542 | Herpotrichiellaceae | Chaetothyriales |
| Capronia semi-immersa | A0A0D2CB06 | 5601 | Herpotrichiellaceae | Chaetothyriales |
| Ceratocystis fimbriata f sp platani | A0A0F8B357 | 88771 | Ceratocystidaceae | Microascales |
| Chaetomium_globosum_strain_ATCC_6205 | Q2GU85 | 306901 | Chaetomiaceae | Sordariales |
| Chaetomium_thermophilum_strain_DSM_1495 | G0S9F6 | 759272 | Chaetomiaceae | Sordariales |
| Cladophialophora_bantiana_CBS_17352 | A0A0D2H164 | 1442370 | Herpotrichiellaceae | Chaetothyriales |
| Cladophialophora carrionii CBS 16054 | V9D2C4 | 1279043 | Herpotrichiellaceae | Chaetothyriales |
| Cladophialophora_psammophila_CBS_110553 | W9VYJ4 | 1182543 | Herpotrichiellaceae | Chaetothyriales |
| Cladophialophora yegresii CBS 114405 | W9VGJ2 | 1182544 | Herpotrichiellaceae | Chaetothyriales |
| Claviceps purpurea strain 201 | M1WDR5 | 1111077 | Clavicipitaceae | Hypocreales |
| Clavispora_lusitaniae_strain_ATCC_42720 | C4Y9B0 | 306902 | Metschnikowiaceae | Saccharomycetales |
| Coccidioides posadasii strain C735 | C5PF60 | 222929 | Onygenales incertae | Onygenales |
| | | | sedis | |
| Cochliobolus_heterostrophus_strain_C5 | M2URM4 | 701091 | Pleosporaceae | Pleosporineae |
| Cochliobolus_sativus_strain_ND90Pr | M2QUN4 | 665912 | Pleosporaceae | Pleosporineae |
| Colletotrichum fioriniae PJ7 | A0A010Q0K6 | 1445577 | Glomerellaceae | Glomerellales |
| Colletotrichum_gloeosporioides_strain_Cg-14 | T0K3N5 | 1237896 | Glomerellaceae | Glomerellales |
| Colletotrichum_gloeosporioides_strain_Nara gc5 | L2FCZ0 | 1213859 | Glomerellaceae | Glomerellales |
| Coniosporium_apollinis_strain_CBS_100218 | R7YPZ5 | 1168221 | Herpotrichiellaceae | Chaetothyriales |
| Cordyceps_brongniartii_RCEF_3172 | A0A167IHY8 | 1081107 | Cordycipitaceae | Hypocreales |
| Cordyceps confragosa | A0A179ILG3 | 1105325 | Cordycipitaceae | Hypocreales |
| Cordyceps confragosa RCEF 1005 | A0A168IZL0 | 1081108 | Cordycipitaceae | Hypocreales |
| Cordyceps militaris strain CM01 | G3JKW0 | 983644 | Cordycipitaceae | Hypocreales |
| Cyberlindnera_fabianii | A0A061AJE3 | 36022 | Phaffomycetaceae | Saccharomycetales |
| Cyberlindnera_jadinii | A0A0H5BZE0 | 4903 | Phaffomycetaceae | Saccharomycetales |
| Cyphellophora europaea CBS 101466 | W2S4E2 | 1220924 | Cyphellophoraceae | Chaetothyriales |
| Debaryomyces fabryi | A0A0V1PSR1 | 58627 | Debaryomycetaceae | Saccharomycetales |
| Debaryomyces_hansenii_strain_ATCC_36239 | Q6BYC0 | 284592 | Debaryomycetaceae | Saccharomycetales |
| Diaporthe_ampelina | A0A0G2FGT3 | 1214573 | Diaporthaceae | Diaporthales |
| Didymella_rabiei | A0A163BXA9 | 5454 | Didymellaceae | Pleosporineae |
| Diplodia seriata | A0A0G2E461 | 420778 | Botryosphaeriaceae | Botryosphaeriales |
| Dothistroma septosporum strain NZE10 | N1Q4Q2 | 675120 | Mycosphaerellaceae | Capnodiales |
| Drechmeria coniospora | A0A151GM17 | 98403 | Ophiocordycipitaceae | Hypocreales |
| Drechslerella stenobrocha 248 | W7I376 | 1043628 | Orbiliaceae | Orbiliales |
| Emericella nidulans | Q7SI72 | 162425 | Aspergillaceae | Eurotiales |
| Emmonsia crescens UAMH 3008 | A0A0G2J9S8 | 1247875 | Ajellomycetaceae | Onygenales |
| Emmonsia_parva_UAMH_139 | A0A0H1BAF5 | 1246674 | Ajellomycetaceae | Onygenales |
| Endocarpon_pusilium_strain_Z07020 | U1HY26 | 1263415 | Verrucariaceae | Verrucariales |
| Eremothecium cymbalariae | G0XP51 | 45285 | Saccharomycetaceae | Saccharomycetales |
| Eremothecium_cymbalariae_strain_CBS_27075 | G8JMH5 | 931890 | Saccharomycetaceae | Saccharomycetales |
| Eremothecium sinecaudum | A0A0X8HRQ0 | 45286 | Saccharomycetaceae | Saccharomycetales |
| Escovopsis_weberi | A0A0M8MV01 | 150374 | Hypocreaceae | Hypocreales |
| Eutypa_lata_strain_UCR-EL1 | M7T4F8 | 1287681 | Diatrypaceae | Xylariales |
| Exophiala aquamarina CBS 119918 | A0A072PDE7 | 1182545 | Herpotrichiellaceae | Chaetothyriales |
| Exophiala_dermatitidis_strain_ATCC_34100 | H6BSM7 | 858893 | Herpotrichiellaceae | Chaetothyriales |
| Exophiala mesophila | A0A0D1X796 | 212818 | Herpotrichiellaceae | Chaetothyriales |
| Exophiala_oligosperma | A0A0D2DBN2 | 215243 | Herpotrichiellaceae | Chaetothyriales |
| Exophiala_sideris | A0A0D1YM75 | 1016849 | Herpotrichiellaceae | Chaetothyriales |
| Exophiala spinifera | A0A0D1YGB1 | 91928 | Herpotrichiellaceae | Chaetothyriales |
| Exophiala xenobiotica | A0A0D2C0F9 | 348802 | Herpotrichiellaceae | Chaetothyriales |
| Fonsecaea erecta | A0A178Z6Z0 | 1367422 | Herpotrichiellaceae | Chaetothyriales |
| Fonsecaea_monophora | A0A177F142 | 254056 | Herpotrichiellaceae | Chaetothyriales |
| Fonsecaea_multimorphosa | A0A178BUX8 | 979981 | Herpotrichiellaceae | Chaetothyriales |
| Fonsecaea multimorphosa CBS 102226 | A0A0D2JMN8 | 1442371 | Herpotrichiellaceae | Chaetothyriales |
| Fonsecaea nubica | A0A178DBT6 | 856822 | Herpotrichiellaceae | Chaetothyriales |
| Fonsecaea pedrosoi CBS 27137 | A0A0D2EJA9 | 1442368 | Herpotrichiellaceae | Chaetothyriales |
| Fusarium langsethiae | A0A0N0DGM2 | 179993 | Nectriaceae | Hypocreales |
| Fusarium_oxysporum_f_sp_cubense_strain race 1 | N4UWI3 | 1229664 | Nectriaceae | Hypocreales |
| Fusarium_oxysporum_f_sp_cubense_strain race 4 | N1RVA8 | 1229665 | Nectriaceae | Hypocreales |
| Fusarium_oxysporum_f_sp_cubense_trop- | X0KQL5 | 1089451 | Nectriaceae | Hypocreales |
| ical_race_4_54006 | | | | |
| Fusarium_oxysporum_f_sp_lycopersici_strain_4287 | A0A0D2Y2Y4 | 426428 | Nectriaceae | Hypocreales |
| Fusarium_oxysporum_f_sp_melonis_26406 | X0AAF8 | 1089452 | Nectriaceae | Hypocreales |
| Fusarium oxysporum f sp pisi HDV247 | W9PM09 | 1080344 | Nectriaceae | Hypocreales |
| Fusarium_oxysporum_f_sp_raphani_54005 | X0CCQ3 | 1089458 | Nectriaceae | Hypocreales |
| Fusarium_oxysporum_Fo47 | W9K2M0 | 660027 | Nectriaceae | Hypocreales |
| Fusarium_oxysporum_FOSC_3-a | W9IAH9 | 909455 | Nectriaceae | Hypocreales |
| Fusarium oxysporum strain Fo5176 | F9F4J6 | 660025 | Nectriaceae | Hypocreales |
| Fusarium_pseudograminearum_strain_CS3096 | K3V2E5 | 1028729 | Nectriaceae | Hypocreales |
| Gaeumannomyces_graminis_var_tritici_strain R3-111a-1 | J3P889 | 644352 | Magnaporthaceae | Magnaporthales |
| Geotrichum_candidum | A0A0J9X829 | 1173061 | Dipodascaceae | Saccharomycetales |
| Gibberella_fujikuroi | A0A0J0BY83 | 5127 | Nectriaceae | Hypocreales |
| Gibberella fujikuroi strain CBS 19534 | S0E2K7 | 1279085 | Nectriaceae | Hypocreales |
| Gibberella moniliformis strain M3125 | W7MQM8 | 334819 | Nectriaceae | Hypocreales |
| Gibberella zeae strain PH-1 | I1RG07 | 229533 | Nectriaceae | Hypocreales |
| Glarea_lozoyensis_strain_ATCC_20868 | S3DBU4 | 1116229 | Helotiaceae | Helotiales |
| Grosmannia_clavigera_strain_kw1407 | F0XDY3 | 655863 | Ophiostomataceae | Ophiostomatales |
| Hanseniaspora uvarum DSM 2768 | A0A0F4XDF5 | 1246595 | Saccharomycodaceae | Saccharomycetales |
| Hypocrea_atroviridis_strain_ATCC_20476 | G9NY94 | 452589 | Hypocreaceae | Hypocreales |
| Hypocrea jecorina | G9IJ58 | 51453 | Hypocreaceae | Hypocreales |
| Hypocrea jecorina strain ATCC 56765 | A0A024S6P5 | 1344414 | Hypocreaceae | Hypocreales |
| Hypocrea jecorina strain QM6a | G0RMK2 | 431241 | Hypocreaceae | Hypocreales |
| Hypocrea virens strain Gv29-8 | G9MQ44 | 413071 | Hypocreaceae | Hypocreales |
| Hypocrella_siamensis | A0A172Q4C2 | 696354 | Clavicipitaceae | Hypocreales |
| Isaria_fumosorosea_ARSEF_2679 | A0A167XIR1 | 1081104 | Cordycipitaceae | Hypocreales |
| Kazachstania_africana_strain_ATCC_22294 | H2ASI7 | 1071382 | Saccharomycetaceae | Saccharomycetales |
| Kazachstania_naganishii_strain_ATCC_MYA-139 | J7RM21 | 1071383 | Saccharomycetaceae | Saccharomycetales |
| Kluyveromyces dobzhanskii CBS 2104 | A0A0A8LC24 | 1427455 | Saccharomycetaceae | Saccharomycetales |
| Kluyveromyces_lactis_strain_ATCC_8585 | Q6CIP0 | 284590 | Saccharomycetaceae | Saccharomycetales |
| Kluyveromyces_marxianus_DMKU3-1042 | W0TFI2 | 1003335 | Saccharomycetaceae | Saccharomycetales |
| Komagataella pastoris strain GS115 | C4R6X5 | 644223 | Phaffomycetaceae | Saccharomycetales |
| Kuraishia capsulata CBS 1993 | W6MJ91 | 1382522 | Saccharomycetales | Saccharomycetales |
| | | | incertae sedis | |
| Lachancea kluyveri | P12384 | 4934 | Saccharomycetaceae | Saccharomycetales |
| Lachancea_lanzarotensis | A0A0C7N6G7 | 1245769 | Saccharomycetaceae | Saccharomycetales |
| Lachancea_quebecensis | A0A0P1KZX7 | 1654605 | Saccharomycetaceae | Saccharomycetales |
| Lachancea_thermotolerans_strain_ATCC 56472 | C5DBK0 | 559295 | Saccharomycetaceae | Saccharomycetales |
| Leptosphaeria maculans strain JN3 | E5A529 | 985895 | Leptosphaeria | Pleosporineae |
| Lodderomyces_elongisporus_strain_ATCC 11503 | A5E1D9 | 379508 | Debaryomycetaceae | Saccharomycetales |
| Macrophomina_phaseolina_strain_MS6 | K2S5Z6 | 1126212 | Botryosphaeriaceae | Botryosphaeriales |
| Madurella_mycetomatis | A0A175W3I2 | 100816 | mitosporic Sordariales | Sordariales |
| Magnaporthe oryzae strain 70-15 | G4MR89 | 242507 | Magnaporthaceae | Magnaporthales |
| Magnaporthe oryzae strain Y34 | L7HVB4 | 1143189 | Magnaporthaceae | Magnaporthales |
| Magnaporthiopsis_poae_strain_ATCC_64411 | A0A0C4DS73 | 644358 | Magnaporthaceae | Magnaporthales |
| Marssonina_brunnea_f_sp_multigermtubi strain MB m1 | K1X8D8 | 1072389 | Dermateaceae | Helotiales |
| Metarhizium acridum strain CQMa 102 | E9DXW9 | 655827 | Clavicipitaceae | Hypocreales |
| Metarhizium album ARSEF 1941 | A0A0B2WQA5 | 1081103 | Clavicipitaceae | Hypocreales |
| Metarhizium_anisopliae_ARSEF_549 | A0A0B4EKU5 | 1276135 | Clavicipitaceae | Hypocreales |
| Metarhizium_anisopliae_BRIP_53293 | A0A0D9NQS0 | 1291518 | Clavicipitaceae | Hypocreales |
| Metarhizium brunneum ARSEF 3297 | A0A0B4FKS3 | 1276141 | Clavicipitaceae | Hypocreales |
| Metarhizium guizhouense ARSEF 977 | A0A0B4H8M1 | 1276136 | Clavicipitaceae | Hypocreales |
| Metarhizium majus ARSEF 297 | A0A0B4HXD6 | 1276143 | Clavicipitaceae | Hypocreales |
| Metarhizium_rileyi_RCEF_4871 | A0A167AMF2 | 1081105 | Clavicipitaceae | Hypocreales |
| Metarhizium_robertsii | A0A014PAK1 | 568076 | Clavicipitaceae | Hypocreales |
| Metarhizium robertsii strain ARSEF 23 | E9EMS3 | 655844 | Clavicipitaceae | Hypocreales |
| Meyerozyma_guilliermondii_strain_ATCC 6260 | A5DFC0 | 294746 | Debaryomycetaceae | Saccharomycetales |
| Naumovozyma_castellii_strain_ATCC_76901 | G0VD13 | 1064592 | Saccharomycetaceae | Saccharomycetales |
| Naumovozyma_dairenensis_strain_ATCC_10597 | G0WE84 | 1071378 | Saccharomycetaceae | Saccharomycetales |
| Nectria_haematococca_strain_77-13-4 | C7ZA34 | 660122 | Nectriaceae | Hypocreales |
| Neonectria ditissima | A0A0P7AWF2 | 78410 | Nectriaceae | Hypocreales |
| Neosartorya fischeri strain ATCC 1020 | A1D5Z2 | 331117 | Aspergillaceae | Eurotiales |
| Neosartorya fumigata strain CEA10 | B0XZZ4 | 451804 | Aspergillaceae | Eurotiales |
| Neurospora_africana | K7ZVW9 | 5143 | Sordariaceae | Sordariales |
| Neurospora_calospora | K7ZWV9 | 165411 | Sordariaceae | Sordariales |
| Neurospora cerealis | K7ZW01 | 29881 | Sordariaceae | Sordariales |
| Neurospora crassa | D2N2E0 | 5141 | Sordariaceae | Sordariales |
| Neurospora crassa strain ATCC 24698 | Q1K6I3 | 367110 | Sordariaceae | Sordariales |
| Neurospora galapagosensis | K7ZWN2 | 88769 | Sordariaceae | Sordariales |
| Neurospora hapsidophora | K7ZW48 | 176947 | Sordariaceae | Sordariales |
| Neurospora intermedia | D2N2E7 | 5142 | Sordariaceae | Sordariales |
| Neurospora_kobi | K7ZVX0 | 241062 | Sordariaceae | Sordariales |
| Neurospora_lineolata | K7ZWW0 | 88717 | Sordariaceae | Sordariales |
| Neurospora novoguineensis | K7ZW03 | 241060 | Sordariaceae | Sordariales |
| Neurospora pannonica | K7ZWN3 | 83678 | Sordariaceae | Sordariales |
| Neurospora retispora | K7ZW49 | 241054 | Sordariaceae | Sordariales |
| Neurospora_santi-florii | K7ZVX1 | 176682 | Sordariaceae | Sordariales |
| Neurospora_sitophila | D2N2F3 | 40126 | Sordariaceae | Sordariales |
| Neurospora sp FGSC 8780 | D2N2G4 | 482004 | Sordariaceae | Sordariales |
| Neurospora sp FGSC 8815 | D2N2F6 | 228687 | Sordariaceae | Sordariales |
| Neurospora sp FGSC 8817 | D2N2F7 | 481997 | Sordariaceae | Sordariales |
| Neurospora_sp_FGSC_8827 | D2N2G3 | 482003 | Sordariaceae | Sordariales |
| Neurospora_sp_FGSC_8842 | D2N2G2 | 482002 | Sordariaceae | Sordariales |
| Neurospora sp FGSC 8853 | D2N2F9 | 481999 | Sordariaceae | Sordariales |
| Neurospora sublineolata | K7ZWW1 | 165293 | Sordariaceae | Sordariales |
| Neurospora terricola | K7ZWN4 | 88718 | Sordariaceae | Sordariales |
| Neurospora_tetrasperma | D2N2F4 | 40127 | Sordariaceae | Sordariales |
| Neurospora_uniporata | K7ZW50 | 241063 | Sordariaceae | Sordariales |
| Ogataea_parapolymorpha_strain_ATCC_26012 | W1QE65 | 871575 | Pichiaceae | Saccharomycetales |
| Oidiodendron maius Zn | A0A0C3HTW3 | 913774 | mitosporic | Leotiomycetes |
| | | | Myxotrichaceae | incertae sedis |
| Ophiocordyceps sinensis strain Co18 | T5A148 | 911162 | Ophiocordycipitaceae | Hypocreales |
| Ophiocordyceps unilateralis | A0A0L9SIN1 | 268505 | Ophiocordycipitaceae | Hypocreales |
| Ophiostoma_piceae_strain_UAMH_11346 | S3C5N9 | 1262450 | Ophiostomataceae | Ophiostomatales |
| Paracoccidioides_brasiliensis_strain_Pb03 | C0SDN9 | 482561 | Onygenales incertae | Onygenales |
| | | | sedis | |
| Paracoccidioides_brasiliensis_strain_Pb18 | C1GFU7 | 502780 | Onygenales incertae | Onygenales |
| | | | sedis | |
| Paracoccidioides_lutzii_strain_ATCC_MYA-826 | C1H517 | 502779 | Onygenales incertae | Onygenales |
| | | | sedis | |
| Paraphaeosphaeria sporulosa | A0A177CPX6 | 1460663 | Didymosphaeriaceae | Massarineae |
| Penicillium brasilianum | A0A0F7TPZ2 | 104259 | Aspergillaceae | Eurotiales |
| Penicillium camemberti FM 013 | A0A0G4P840 | 1429867 | Aspergillaceae | Eurotiales |
| Penicillium_chrysogenum | B1GVB8 | 5076 | Aspergillaceae | Eurotiales |
| Penicillium_digitatum_strain_PHI26 | K9G3Z6 | 1170229 | Aspergillaceae | Eurotiales |
| Penicillium expansum | A0A0A2K1S7 | 27334 | Aspergillaceae | Eurotiales |
| Penicillium freii | A0A101MNI9 | 48697 | Aspergillaceae | Eurotiales |
| Penicillium italicum | A0A0A2LAS4 | 40296 | Aspergillaceae | Eurotiales |
| Penicillium_nordicum | A0A0M8PFN9 | 229535 | Aspergillaceae | Eurotiales |
| Penicillium_oxalicum_strain_114-2 | S7Z940 | 933388 | Aspergillaceae | Eurotiales |
| Penicillium patulum | A0A135LCC8 | 5078 | Aspergillaceae | Eurotiales |
| Penicillium roqueforti strain FM164 | W6PVN7 | 1365484 | Aspergillaceae | Eurotiales |
| Pestalotiopsis fici W106-1 | W3XDQ7 | 1229662 | Sporocadaceae | Xylariales |
| Phaeomoniella_chlamydospora | A0A0G2HF89 | 158046 | Phaeomoniellales | Phaeomoniellales |
| | | | incertae sedis | |
| Phaeosphaeria_nodorum_strain_SN15 | Q0UCT8 | 321614 | Phaeosphaeriaceae | Pleosporineae |
| Pichia kudriavzevii | A0A099NXR5 | 4909 | Pichiaceae | Saccharomycetales |
| Pichia_sorbitophila_strain_ATCC_MYA-4447 | G8YMJ7 | 559304 | Debaryomycetaceae | Saccharomycetales |
| Pichia_sorbitophila_strain_ATCC_MYA-4447 | G8YMZ0 | 559304 | Debaryomycetaceae | Saccharomycetales |
| Pneumocystis carinii | A2TJ26 | 4754 | Pneumocystidaceae | Pneumocystidomy |
| | | | | cetes |
| Pneumocystis carinii B80 | A0A0W4ZHE5 | 1408658 | Pneumocystidaceae | Pneumocystidomy |
| | | | | cetes |
| Pneumocystis jiroveci strain SE8 | L0PDU6 | 1209962 | Pneumocystidaceae | Pneumocystidomy |
| | | | | cetes |
| Pneumocystis_jirovecii_RU7 | A0A0W4ZVY3 | 1408657 | Pneumocystidaceae | Pneumocystidomy |
| | | | | cetes |
| Pneumocystis_murina_strain_B123 | M7P3B3 | 1069680 | Pneumocystidaceae | Pneumocystidomy |
| | | | | cetes |
| Pochonia chlamydosporia 170 | A0A179FF27 | 1380566 | Clavicipitaceae | Hypocreales |
| Podospora anserina strain S | B2ADL1 | 515849 | Lasiosphaeriaceae | Sordariales |
| Pseudocercospora_fijiensis_strain_CIRAD86 | N1Q996 | 383855 | Mycosphaerellaceae | Capnodiales |
| Pseudogymnoascus_destructans | A0A177ADM2 | 655981 | Pseudeurotiaceae | Leotiomycetes |
| | | | | incertae sedis |
| Pseudogymnoascus_destructans_strain_ATCC_MYA-4855 | L8G637 | 658429 | Pseudeurotiaceae | Leotiomycetes |
| | | | | incertae sedis |
| Pseudogymnoascus sp VKM F-103 | A0A094E1R1 | 1420912 | Pseudeurotiaceae | Leotiomycetes |
| | | | | incertae sedis |
| Pseudogymnoascus sp VKM F-3557 | A0A093XIK8 | 1437433 | Pseudeurotiaceae | Leotiomycetes |
| | | | | incertae sedis |
| Pseudogymnoascus sp VKM F-3775 | A0A094AA23 | 1420901 | Pseudeurotiaceae | Leotiomycetes |
| | | | | incertae sedis |
| Pseudogymnoascus_sp_VKM_F-3808 | A0A093YGI7 | 1391699 | Pseudeurotiaceae | Leotiomycetes |
| | | | | incertae sedis |
| Pseudogymnoascus_sp_VKM_F-4246 | A0A093Z5B5 | 1420902 | Pseudeurotiaceae | Leotiomycetes |
| | | | | incertae sedis |
| Pseudogymnoascus_sp_VKM_F-4281 FW-2241 | A0A094CRD8 | 1420906 | Pseudeurotiaceae | Leotiomycetes |
| | | | | incertae sedis |
| Pseudogymnoascus_sp_VKM_F-4513 FW-928 | A0A094BQ07 | 1420907 | Pseudeurotiaceae | Leotiomycetes |
| | | | | incertae sedis |
| Pseudogymnoascus_sp_VKM_F-4515 FW-2607 | A0A094FEM7 | 1420909 | Pseudeurotiaceae | Leotiomycetes |
| | | | | incertae sedis |
| Pseudogymnoascus_sp_VKM_F-4516_FW-969 | A0A094CTP6 | 1420910 | Pseudeurotiaceae | Leotiomycetes |
| | | | | incertae sedis |
| Pseudogymnoascus_sp_VKM_F-4517 FW-2822 | A0A094FK10 | 1420911 | Pseudeurotiaceae | Leotiomycetes |
| | | | | incertae sedis |
| Pseudogymnoascus_sp_VKM_F-4518 FW-2643 | A0A094ET92 | 1420913 | Pseudeurotiaceae | Leotiomycetes |
| | | | | incertae sedis |
| Pseudogymnoascus_sp_VKM_F-4519 FW-2642 | A0A094K4N9 | 1420914 | Pseudeurotiaceae | Leotiomycetes |
| | | | | incertae sedis |
| Pseudogymnoascus_sp_VKM_F-4520 FW-2644 | A0A094JHH7 | 1420915 | Pseudeurotiaceae | Leotiomycetes |
| | | | | incertae sedis |
| Purpureocillium lilacinum | A0A179GB12 | 33203 | Ophiocordycipitaceae | Hypocreales |
| Pyrenochaeta sp DS3sAY3a | A0A178DZ21 | 765867 | Cucurbitariaceae | Pleosporineae |
| Pyrenophora teres f teres strain 0-1 | E3RI43 | 861557 | Pleosporaceae | Pleosporineae |
| Pyrenophora_tritici-repentis_strain_Pt-1C-BFP | B2WIP5 | 426418 | Pleosporaceae | Pleosporineae |
| Pyronema_omphalodes_strain_CBS_100304 | U4LPJ5 | 1076935 | Pyronemataceae | Pezizales |
| Rasamsonia emersonii CBS 39364 | A0A0F4YHC8 | 1408163 | Trichocomaceae | Eurotiales |
| Rhinocladiella mackenziei CBS 65093 | A0A0D2H556 | 1442369 | Herpotrichiellaceae | Chaetothyriales |
| Saccharomyces arboricola strain H-6 | J8Q5L6 | 1160507 | Saccharomycetaceae | Saccharomycetales |
| Saccharomyces_bayanus | Q8J1R6 | 4931 | Saccharomycetaceae | Saccharomycetales |
| Saccharomyces_cerevisiae_strain_ATCC_204508 | D6VTK4 | 559292 | Saccharomycetaceae | Saccharomycetales |
| Saccharomyces_cerevisiae_strain_AWRI796 | E7KC22 | 764097 | Saccharomycetaceae | Saccharomycetales |
| Saccharomyces_cerevisiae_strain_FostersO | E7NH73 | 764101 | Saccharomycetaceae | Saccharomycetales |
| Saccharomyces_cerevisiae_strain_RM11-1a | B3LUI5 | 285006 | Saccharomycetaceae | Saccharomycetales |
| Saccharomyces_cerevisiae_strain_YJM789 | A7A213 | 307796 | Saccharomycetaceae | Saccharomycetales |
| Saccharomyces_cerevisiae_×_Saccha- | H0GU93 | 1095631 | Saccharomycetaceae | Saccharomycetales |
| romyces_kudriavzevii_strain_VIN7 | | | | |
| Saccharomyces paradoxus | Q8J080 | 27291 | Saccharomycetaceae | Saccharomycetales |
| Saccharomyces pastorianus | Q8J1Q4 | 27292 | Saccharomycetaceae | Saccharomycetales |
| Saccharomyces sp ‘boulardii’ | A0A0L8VRV2 | 252598 | Saccharomycetaceae | Saccharomycetales |
| Saitoella_complicata_NRRL_Y-17804 | A0A0E9NKH5 | 698492 | Protomycetaceae | Taphrinales |
| Scedosporium_apiospermum | A0A084FZY6 | 563466 | Microascaceae | Microascales |
| Scheffersomyces_stipitis_strain_ATCC_58785 | A3LXU7 | 322104 | Debaryomycetaceae | Saccharomycetales |
| Schizosaccharomyces_cryophilus_strain_OY26 | S9VVX5 | 653667 | Schizosaccharomycetaceae | Schizosaccharomycetales |
| Schizosaccharomyces_japonicus_strain_yFS275 | B6JZE2 | 402676 | Schizosaccharomycetaceae | Schizosaccharomycetales |
| Schizosaccharomyces_octosporus_strain_yFS286 | S9PVP9 | 483514 | Schizosaccharomycetaceae | Schizosaccharomycetales |
| Schizosaccharomyces pombe strain 972 | Q00619 | 284812 | Schizosaccharomycetaceae | Schizosaccharomycetales |
| Sclerotinia borealis F-4157 | W9C8T9 | 1432307 | Sclerotiniaceae | Helotiales |
| Sclerotinia_sclerotiorum_strain_ATCC_18683 | A7EY95 | 665079 | Sclerotiniaceae | Helotiales |
| Setosphaeria_turcica_strain_28A | R0KC11 | 671987 | Pleosporaceae | Pleosporineae |
| Sordaria_macrospora_strain_ATCC_MYA-333 | F7W5S1 | 771870 | Sordariaceae | Sordariales |
| Spathaspora_passalidarum_strain_NRRLY-27907 | G3AJU2 | 619300 | Debaryomycetaceae | Saccharomycetales |
| Sphaerulina musiva strain SO2202 | N1QN82 | 692275 | Mycosphaerellaceae | Capnodiales |
| Sporothrix_brasiliensis_5110 | A0A0C2IIS5 | 1398154 | Ophiostomataceae | Ophiostomatales |
| Sporothrix_insectorum_RCEF_264 | A0A162MTF1 | 1081102 | Ophiostomataceae | Ophiostomatales |
| Sporothrix schenckii | H9XTI1 | 29908 | Ophiostomataceae | Ophiostomatales |
| Sporothrix schenckii 1099-18 | A0A0F2M7E2 | 1397361 | Ophiostomataceae | Ophiostomatales |
| Sporothrix_schenckii_strain_ATCC_58251 | U7Q511 | 1391915 | Ophiostomataceae | Ophiostomatales |
| Stachybotrys_chartarum_IBT_40288 | A0A084RP20 | 1283842 | Stachybotriaceae | Hypocreales |
| Stachybotrys_chartarum_IBT_7711 | A0A084ASH4 | 1280523 | Stachybotriaceae | Hypocreales |
| Stachybotrys chlorohalonata IBT 40285 | A0A084QT65 | 1283841 | Stachybotriaceae | Hypocreales |
| Stagonospora sp SRC1lsM3a | A0A178ACM9 | 765868 | Massarinaceae | Massarineae |
| Stemphylium lycopersici | A0A0L1HGK2 | 183478 | Pleosporaceae | Pleosporineae |
| Sugiyamaella_{—}lignohabitans | A0A161HL65 | 796027 | Trichomonascaceae | Saccharomycetales |
| Talaromyces_{—}islandicus | A0A0U1LRR7 | 28573 | Trichocomaceae | Eurotiales |
| Talaromyces marneffei PM1 | A0A093XYN6 | 1077442 | Trichocomaceae | Eurotiales |
| Talaromyces_marneffei_strain_ATCC_18224 | B6Q4A9 | 441960 | Trichocomaceae | Eurotiales |
| Talaromyces_stipitatus_strain_ATCC_10500 | B8M557 | 441959 | Trichocomaceae | Eurotiales |
| Tetrapisispora_blattae_strain_ATCC_34711 | I2H305 | 1071380 | Saccharomycetaceae | Saccharomycetales |
| Tetrapisispora_phaffii_strain_ATCC_24235 | G8C206 | 1071381 | Saccharomycetaceae | Saccharomycetales |
| Togninia minima strain UCR-PA7 | R8BGY4 | 1286976 | Togniniaceae | Togniniales |
| Tolypocladium_ophioglossoides_CBS_100239 | A0A0L0N0N3 | 1163406 | Ophiocordycipitaceae | Hypocreales |
| Torrubiella_hemipterigena | A0A0A1SZJ6 | 1531966 | Clavicipitaceae | Hypocreales |
| Torulaspora_delbrueckii_strain_ATCC_10662 | G8ZR18 | 1076872 | Saccharomycetaceae | Saccharomycetales |
| Trichoderma gamsii | A0A0W7VR33 | 398673 | Hypocreaceae | Hypocreales |
| Trichoderma harzianum | A0A0F9XI50 | 5544 | Hypocreaceae | Hypocreales |
| Trichophyton_equinum_strain_ATCC_MYA-4606 | F2PNP9 | 559882 | Arthrodermataceae | Onygenales |
| Trichophyton_interdigitale_MR816 | A0A059J435 | 1215338 | Arthrodermataceae | Onygenales |
| Trichophyton rubrum | A0A178ETN9 | 5551 | Arthrodermataceae | Onygenales |
| Trichophyton rubrum CBS 28886 | A0A022VRI2 | 1215330 | Arthrodermataceae | Onygenales |
| Trichophyton_verrucosum_strain_HKI_0517 | D4DBK6 | 663202 | Arthrodermataceae | Onygenales |
| Trichophyton_violaceum | A0A178FB33 | 34388 | Arthrodermataceae | Onygenales |
| Tuber_melanosporum_strain_Mel28 | D5GJK5 | 656061 | Tuberaceae | Pezizales |
| Uncinocarpus_reesii_strain_UAMH_1704 | C4JL18 | 336963 | Onygenaceae | Onygenales |
| Uncinula necator | A0A0B1P9N6 | 52586 | Erysiphaceae | Erysiphales |
| Ustilaginoidea virens | A0A063BN49 | 1159556 | Hypocreales incertae | Hypocreales |
| | | | sedis | |
| Vanderwaltozyma_polyspora_strain_ATCC_22028 | A7TJQ6 | 436907 | Saccharomycetaceae | Saccharomycetales |
| Vanderwaltozyma_polyspora_strain_ATCC_22028 | A7TQX4 | 436907 | Saccharomycetaceae | Saccharomycetales |
| Verruconis gallopava | A0A0D2AMB2 | 253628 | Sympoventuriaceae | Venturiales |
| Verticillium alfalfae strain VaMs102 | C9SGY3 | 526221 | Plectosphaerellaceae | Glomerellales |
| Verticillium dahliae strain VdLs17 | G2X5W7 | 498257 | Plectosphaerellaceae | Glomerellales |
| Verticillium longisporum | A0A0G4M417 | 100787 | Plectosphaerellaceae | Glomerellales |
| Wickerhamomyces_ciferrii_strain_F-60-10 | K0KPE3 | 1206466 | Phaffomycetaceae | Saccharomycetales |
| Xylona heveae TC161 | A0A165HIN9 | 1328760 | Xylonomycetaceae | Xylonomycetales |
| Yarrowia_lipolytica_strain_CLIB_122 | Q6C2Z3 | 284591 | Dipodascaceae | Saccharomycetales |
| Zygosaccharomyces_bailii_ISA1307 | W0VI75 | 1355161 | Saccharomycetaceae | Saccharomycetales |
| Zygosaccharomyces_bailii_strain_CLIB_213 | S6EXB4 | 1333698 | Saccharomycetaceae | Saccharomycetales |
| Zygosaccharomyces_rouxii_strain_ATCC 2623 | C5DX97 | 559307 | Saccharomycetaceae | Saccharomycetales |
| Zymoseptoria brevis | A0A0F4GDL4 | 1047168 | Mycosphaerellaceae | Capnodiales |
| Zymoseptoria_tritici_strain_CBS_115943 | F9X131 | 336722 | Mycosphaerellaceae | Capnodiales |

In certain embodiments, the GPCR or a portion thereof for use in the present disclosure comprises an amino acid sequence or a nucleotide sequence that has greater than about 15% homology to any one of the GPCRs disclosed herein and further comprises a characteristic seven transmembrane helix domain. For example, but not by way of limitation, the GPCR or a portion thereof comprises an amino acid sequence that has greater than about 15% homology to an amino acid sequence comprising any one of SEQ ID NOs: 117-161 or an amino acid sequence of any one of the GPCRs listed in Table 11 and further comprises a characteristic seven transmembrane helix domain. In certain embodiments, the GPCR or a portion thereof comprises a nucleotide sequence that has greater than about 15% homology to a nucleotide sequence comprising any one of SEQ ID NOs: 168-211 and further comprises a characteristic seven transmembrane helix domain. In certain embodiments, the GPCR or a portion thereof for use in the present disclosure comprises an amino acid sequence that has greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 91%, greater than about 92%, greater than about 93%, greater than about 94%, greater than about 95%, greater than about 96%, greater than about 97%, greater than about 98% or greater than about 99% homology to any one of the GPCRs disclosed herein and further comprises a characteristic seven transmembrane helix domain. For example, but not by way of limitation, the GPCR or a portion thereof comprises an amino acid greater than about 15% homology, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 91%, greater than about 92%, greater than about 93%, greater than about 94%, greater than about 95%, greater than about 96%, greater than about 97%, greater than about 98% or greater than about 99% homology to an amino acid sequence comprising any one of SEQ ID NOs: 117-161 or an amino acid sequence of any one of the GPCRs listed in Table 11 and further comprises a characteristic seven transmembrane helix domain.

In certain embodiments, the GPCR is a variant of the yeast Ste2 receptor or Ste3 receptor. The mating factor receptors Ste2 and Ste3 are integral membrane proteins that can be involved in the response to mating factors on the cell membrane. The Ste2 subfamily represents the alpha-factor peptide pheromone receptor encoded by the Ste2 gene, and the Ste3 subfamily represents the a-factor peptide pheromone receptor encoded by the Ste3 gene, which are required for peptide pheromone sensing and mating in haploid cells of the yeast Saccharomyces cerevisiae. The Ste2-encoded and Ste3-encoded seven-transmembrane domain receptors are the two major subfamily members of the class D GPCRs. Ste2 and Ste3 GPCRs sense the peptide mating pheromones, alpha-factor and a-factor, which activate a GPCR on the surface of the opposite yeast-mating haploid-types (MATa and MAT-alpha), respectively. In certain embodiments, the Ste2 receptor or Ste3 receptor is modified so that it binds to a ligand disclosed herein rather than a yeast pheromone. For example, but not by way of limitation, the GPCR or portion thereof is a polypeptide that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to the native yeast Ste2 or yeast Ste3 receptor.

In certain embodiments, a homolog of a nucleotide sequence can be a polynucleotide having changes in one or more nucleotide bases that can result in substitution of one or more amino acids, but do not affect the functional properties of the polypeptide or protein encoded by the nucleotide sequence. Homologs can also include polynucleotides having modifications such as deletion, addition or insertion of nucleotides that do not substantially affect the functional properties of the resulting polynucleotide or transcript. Alterations in a polynucleotide that result in the production of a chemically equivalent amino acid at a given site, but do not affect the functional properties of the encoded polypeptide, are well known in the art.

In certain embodiments, a homolog of a peptide, polypeptide or protein can be a peptide, polypeptide or protein having changes in one or more amino acids but do not affect the functional properties of the peptide, polypeptide or protein. Alterations in a peptide, polypeptide or protein that do not affect the functional properties of the peptide, polypeptide or protein, are well known in the art, e.g., conservative substitutions. It is therefore understood that the disclosure encompasses more than the specific exemplary polynucleotide or amino acid sequences and includes functional equivalents thereof.

Conservative substitutions are shown in Table 1, under the heading of “conservative substitutions.” More substantial changes are also provided in Table 1 under the heading of “exemplary substitutions,” and as further described below in reference to amino acid side chain classes.

TABLE 1

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids can be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for a member of another class.

In certain embodiments, GPCRs for use in the present disclosure are identified by searching a protein and/or genomic database and/or literature for a protein and/or a gene with homology to the S. cerevisiae Ste2 receptor and/or Ste3 receptor, e.g., the identified GPCR has an amino acid sequence that is at least about 15%, e.g., at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98% or at least about 99%, homologous to the S. cerevisiae Ste2 receptor and/or Ste3 receptor.

In certain embodiments, GPCRs for use in the present disclosure are identified by searching a protein and/or genomic database and/or literature for a protein and/or a gene with homology to any of the GPCRs disclosed herein. For example, but not by way of limitation, the identified GPCR can have an amino acid sequence that is at least about 15% homologous, e.g., at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98% or at least about 99%, homologous to a GPCR comprising an amino acid sequence of any one of SEQ ID NOs: 117-161, a GPCR provided in Table 11 and/or a GPCR encoded by a nucleotide sequence comprising any one of SEQ ID NOs: 168-211.

In certain embodiments, the protein and/or genomic database is selected from the group consisting of NCBI, Genbank, Interpro, PFAM, Uniprot and a combination thereof.

GPCR Ligands

The present disclosure further provides ligands (referred to herein as a “GPCR ligand”) configured to interact with (directly and/or indirectly) and activate a GPCR disclosed herein. For example, but not by way of limitation, a GPCR ligand of the present disclosure selectively interacts with a single GPCR allowing activation of the single GPCR in the presence of two or more GPCRs, e.g., where each distinct GPCR is expressed by a separate cell or in the same cell.

In certain embodiments, the ligand can be any molecule that is configured to interact with and activate a GPCR disclosed herein or a GPCR identified by the methods disclosed herein, e.g., by genome mining. For example, but not by way of limitation, the ligand can be a peptide, a protein or portion thereof and/or a small molecule (e.g., nucleotides, lipids, chemicals, toxins, photons, electrical signals and compounds). Non-limiting examples of small molecules include pinene, serotonin and hydroxystrictosidine. See, e.g., Ehrenworth et al., Biochemistry 56(41):5471-5475 (2017), which is incorporated herein in its entirety. Additional examples of ligands for use in the present disclosure is provided in Tables 1 and 2 of Muratspahic et al., Nature-Derived Peptides: A Growing Niche for GPCR Ligand Discovery, Trends in Pharmacological Sciences (2019), in Supplementary Table 3 of Sriram and Insel, GPCRs as targets for approved drugs: How many targets and how many drugs?, Molecular Pharmacology, mol.117.111062 (2018) and in Tables 2, 3 and 5 of U.S. Publication No. 2017/0336407, the contents of which are incorporated herein in their entireties.

In certain embodiments, the ligand is a peptide ligand (referred to herein as a “GPCR peptide ligand”). In certain embodiments, the peptide ligand is secretable (referred to herein as a “secretable GPCR peptide ligand”). For example, but not by way of limitation, the peptide ligand can be expressed intracellularly in a cell and subsequently transported to the plasma membrane of the cell and secreted to the exterior of the cell, e.g., outside the plasma membrane of the cell. In certain embodiments, the peptide is secretable because the peptide is coupled to a secretion signal sequence. In certain embodiments, secretion can be performed using the conserved secretory pathway in yeast.

In certain embodiments, the GPCR peptide ligand, e.g., secretable GPCR peptide ligand, comprises a peptide identified and/or derived from the genome of a species of the phylum Ascomycota. Non-limiting examples of such species include Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, and Capronia coronate.

In certain embodiments, the GPCR peptide ligand, e.g., secretable GPCR peptide ligand, can be composed of about 3-50 amino acid residues. In certain embodiments, the 3-50 amino acid residues can be continuous within a larger polypeptide or protein, or can be a group of 3-50 residues that are discontinuous in a primary sequence of a larger polypeptide or protein but that are spatially near in three-dimensional space. In certain embodiments, the GPCR peptide ligand, e.g., secretable GPCR peptide ligand, can stretch over the complete length of a polypeptide or protein, the GPCR peptide ligand can be part of a peptide, the GPCR peptide ligand can be part of a full protein or polypeptide and can be released from that protein or polypeptide by proteolytic treatment or can remain part of the protein or polypeptide. For example, but not by way of limitation, the GPCR peptide ligand, e.g., secretable GPCR peptide ligand, can be expressed in a cell as part of a longer peptide, e.g., a precursor peptide, that is subsequently processed by proteolytic cleavage to obtain the mature form of the GPCR peptide ligand (see Table 4).

In certain embodiments, the GPCR peptide ligand, e.g., the mature GPCR peptide ligand, can have a length of 3 residues or more, a length of 4 residues or more, a length of 5 residues or more, 6 residues or more, 7, residues or more, 8 residues or more, 9 residues or more, 10 residues or more, 11 residues or more, 12 residues or more, 13 residues or more, 14 residues or more, 15 residues or more, 16 residues or more, 17 residues or more, 18 residues or more, 19 residues or more, 20 residues or more, 21 residues or more, 22 residues or more, 23 residues or more, 24 residues or more, 25 residues or more, 26 residues or more, 27 residues or more, 28 residues or more, 29 residues or more, 30 residues or more, 31 residues or more, 32 residues or more, 33 residues or more, 34 residues or more, 35 residues or more, 36 residues or more, 37 residues or more, 38 residues or more, 39 residues or more, 40 residues or more, 41 residues or more, 42 residues or more, 43 residues or more, 44 residues or more, 45 residues or more, 46 residues or more, 47 residues or more, 48 residues or more, 49 residues or more or 50 residues or more. In certain embodiments, the GPCR peptide ligand has a length of 3-50 residues, 5-50 residues, 3-45 residues, 5-45 residues, 3-40 residues, 5-40 residues, 3-35 residues, 5-35 residues, 3-30 residues, 5-30 residues, 3-25 residues, 5-25 residues, 3-20 residues, 5-20 residues, 3-15 residues, 5-15 residues, 3-10 residues, 3-10 residues, 5-10 residues, 10-15 residues, 15-20 residues, 20-25 residues, 25-30 residues, 30-35 residues, 35-40 residues, 40-45 residues or 45-50 residues. In certain embodiments, the secretable GPCR peptide ligand has a length of about 5 to about 30 residues.

In certain embodiments, the GPCR peptide ligand has a length of 9 residues. In certain embodiments, the GPCR peptide ligand has a length of 10 residues. In certain embodiments, the GPCR peptide ligand has a length of 11 residues. In certain embodiments, the GPCR peptide ligand has a length of 12 residues. In certain embodiments, the GPCR peptide ligand has a length of 13 residues. In certain embodiments, the GPCR peptide ligand has a length of 14 residues. In certain embodiments, the GPCR peptide ligand has a length of 15 residues. In certain embodiments, the GPCR peptide ligand has a length of 16 residues. In certain embodiments, the GPCR peptide ligand has a length of 17 residues. In certain embodiments, the GPCR peptide ligand has a length of 18 residues. In certain embodiments, the GPCR peptide ligand has a length of 19 residues. In certain embodiments, the GPCR peptide ligand has a length of 20 residues. In certain embodiments, the GPCR peptide ligand has a length of 21 residues. In certain embodiments, the GPCR peptide ligand has a length of 22 residues. In certain embodiments, the GPCR peptide ligand has a length of 23 residues. In certain embodiments, the GPCR peptide ligand has a length of 24 residues. In certain embodiments, the GPCR peptide ligand has a length of 25 residues. In certain embodiments, the GPCR peptide ligand has a length of 26 residues. In certain embodiments, the GPCR peptide ligand has a length of 27 residues. In certain embodiments, the GPCR peptide ligand has a length of 28 residues. In certain embodiments, the GPCR peptide ligand has a length of 29 residues. In certain embodiments, the GPCR peptide ligand has a length of 30 residues.

In certain embodiments, the GPCR peptide ligand, e.g., secretable GPCR peptide ligand, or portion thereof can comprise an amino acid sequence of any one of SEQ ID NOs: 1-72, or conservative substitutions thereof or a homolog thereof (see Table 3). In certain embodiments, the GPCR peptide ligand, e.g., secretable GPCR peptide ligand, comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence comprising any one of SEQ ID NOs: 1-72.

In certain embodiments, the GPCR peptide ligand, e.g., secretable GPCR peptide ligand, or portion thereof comprises an amino acid sequence of any one of SEQ ID NOs: 73-116, or conservative substitutions thereof or a homolog thereof (see Table 4). In certain embodiments, the GPCR peptide ligand or portion thereof comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to sequence comprising any one of SEQ ID NOs: 73-116.

In certain embodiments, the GPCR peptide ligand, e.g., secretable GPCR peptide ligand, or portion thereof comprises a nucleotide sequence of any one of SEQ ID NOs: 215-230, or conservative substitutions thereof or a homolog thereof (see Table 7). In certain embodiments, the GPCR peptide ligand, e.g., secretable GPCR peptide ligand, or portion thereof comprises a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a nucleotide sequence comprising any one of SEQ ID NOs: 215-230.

In certain embodiments, the GPCR peptide ligand can comprise a peptide disclosed in Table 12 or conservative substitutions thereof or a homolog thereof. In certain embodiments, the GPCR peptide ligand, e.g., secretable GPCR peptide ligand, or portion thereof comprises a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 12.

In certain embodiments, the GPCR peptide ligand can comprise a peptide disclosed in Tables 2, 3 and 5 of U.S. Publication No. 2017/0336407. For example, but not by way of limitation, the GPCR peptide ligand or portion thereof comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence disclosed in Tables 2, 3 and 5 of U.S. Publication No. 2017/0336407.

In certain embodiments, the GPCR peptide ligand for use in the present disclosure comprises an amino acid sequence or nucleotide sequence that has greater than about 15% homology to any one of the GPCR peptide ligands disclosed herein and further comprises a characteristic pre-pro motif and/or one or more processing sites, as disclosed herein. For example, but not by way of limitation, the GPCR peptide ligand comprises an amino acid sequence that has greater than about 15% homology to an amino acid sequence comprising any one of SEQ ID NOs: 1-116 or an amino acid sequence of any one of the GPCRs peptide ligands listed in Table 12 and further comprises a characteristic pre-pro motif and/or one or more processing sites. In certain embodiments, the GPCR peptide ligand comprises a nucleotide sequence that has greater than about 15% homology to a nucleotide sequence comprising any one of SEQ ID NOs: 215-230 and further comprises a characteristic pre-pro motif and/or one or more processing sites. In certain embodiments, the GPCR peptide ligand thereof for use in the present disclosure comprises an amino acid sequence that has greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 91%, greater than about 92%, greater than about 93%, greater than about 94%, greater than about 95%, greater than about 96%, greater than about 97%, greater than about 98% or greater than about 99% homology to any one of the GPCR peptide ligands disclosed herein and further comprises a characteristic pre-pro motif and/or processing sites. For example, but not by way of limitation, the GPCR peptide ligand comprises an amino acid sequence that has greater than about 15% homology, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 91%, greater than about 92%, greater than about 93%, greater than about 94%, greater than about 95%, greater than about 96%, greater than about 97%, greater than about 98% or greater than about 99% homology to an amino acid sequence comprising any one of SEQ ID NOs: 1-116 or an amino acid sequence of any one of the GPCR peptide ligands listed in Table 12 and further comprises a characteristic pre-pro motif and/or one or more processing sites.

TABLE 12
Non-Limiting Embodiments of Peptide Ligands

| Species | Gene ID | Predicted Peptide Sequence |
|---|---|---|
| Alternaria_brasicicola | ACIW01002317 | WSFTQKRPYGLPIG |
| Arthrobotrys_oligospora | G1X8M4 | WCPYNSCP |
| Ashbya_aceri | R9XEV1 | WHWLRFGDGQSM |
| Ashbya_gossypii | Q752Q1 | WFRLSLHHGQSM |
| Aspergillus_clavatus | A1CLD3 | QWCELPGQGCYMI |
| Aspergillus_flavus | B8NF30 | WCSLPAQGCYML |
| Aspergillus_fumigata | Q4WYU8 | WCHLPGQGCYML |
| Aspergillus_kawachii | G7XMN4 | WCHLPGQPCNMI |
| Aspergillus_nidulans | Q5BAB0 | WCRFAGRICPPT |
| Aspergillus_niger | G3XMV3 | WCVLPGQPCNMI |
| Aspergillus_oryzae | Q2U819 | WCALPGQGC |
| Aspergillus_ruber | A0A017S298 | WCALPGQICS |
| Aspergillus_terreus | Q0CS34 | WCWLPGQGCYML |
| Baudoinia_compniacensis | M2LX19 | GWIGRCGVPGSSC |
| Beauveria_bassiana | J5JMP7 | WCMRPGQPCW |
| Botryosphaeria_parva | R1GET9 | WCRWKGQPCS |
| Botrytis_ciner_ea | G2YE05 | WCGRPGQPC |
| Candida_albicans | Q59Q04 | GFRLTNFGYFEPG |
| Candida_dubliniensis | B9WM67 | KFKLTNFGYFEPG |
| Candida_glabrata | Q6FLY8 | WHWVRLRKGQGLF |
| Candida_guilliermondii | A5DFC0 | KKNSRFLTYWFFQPIM |
| Candida_lusitaniae | C4Y9B0 | WKWIKFRNTDVIG |
| Candida_parapsilosis | G8BFM9 | KPHWTTYGYYEPQ |
| Candida_tenuis | G3BD19 | FSWNYRLKWQPIS |
| Candida_tropicalis | C5M3P6 | KFKFRLTRYGWFSPN |
| Capronia_coronata | W9Y1I9 | LSYWKGVNDGGSS |
| Capronia_epimyces | W9X9V4 | LSYWAGVNDGGSS |
| Chaetomium_globosum | Q2GU85 | WCKQFLGMPCW |
| Chaetomium_thermophilum | G0S9F6 | SWCTRFPGQPCW |
| Chryphonectria_parasitica | O14431 | WCLFHGEGCW |
| Claviceps_purpurea | M1WDR5 | WCWRPGQGCW |
| Coccidioides_immitis | J3KG99 | WCQRPGEPC |
| Colletotrichum_gloeosporioides | T0K3N5 | WCTKPGQPCW |
| Coniosporium_apollinis | R7YPZ5 | WGSRFCHKTGQGCP |
| Dactylellina_haptotyla | S8AWC4 | WCVYNSCP |
| Debaryomyces_hansenii | Q6BYC0 | KFHWMTYRFFQPNL |
| Endocarpon_pusillum | U1HY26 | WWGFRWSRHGTSSW |
| Eremothecium_cymbalariae | G8JMH5 | WHWLRFDRGQPIH |
| Fusarium_oxysporum | F9F4J6 | WCTWRGQPCW |
| Fusarium_pseudograminearum | K3V2E5 | WCTWKGQPCW |
| Gaeumannomyces_graminis | J3P889 | QNGCQYRGQSCW |
| Geotrichum_candidum | A0A024JBH3 | DWGWFWYVPRPGDPAM |
| Gibberella_fujikuroi | S0E2K7 | WCTWRGQPCW |
| Gibberella_moniliformis | W7MQM8 | WCTWRGQPCW |
| Gibberella_zeae | I1RG07 | WCWWKGQPCW |
| Glarea_lozoyensis | S3DBU4 | QCIRHGQPCW |
| Grosmannia_clavigera | F0XDY3 | QWCQWYGQACW |
| Kazachstania_africana | H2ASI7 | WHWLSIAPGQPMYI |
| Kazachstania_naganishii | J7RM21 | WHWLRLSYGQPIY |
| Kluyveromyces_lactis | Q6CIP0 | WSWITLRPGQPIF |
| Kluyveromyces_marxianus | W0TFI2 | WKWLSLRVGQPIY |
| Kluyveromyces_waltii | AADM01000052 | WRWLSLARGQPMY |
| Komagataella_pastorts | F2R066 | FRWRNNEKNQPFG |
| Kuraishia_capsulata | W6MJ91 | RLGARIYAKGQPIY |
| Lachancea_kluyveri | P12384 | WHWLSFSKGEPMY |
| Lachancea_thermotolerans | C5DBK0 | WRWLSLSRGQPMY |
| Lodderomyces_elongisporus | A5E1D9 | WMWTRYGRFSPV |
| Magnaporthe_oryzae | G4MR89 | QWCPRRGQPCW |
| Magnaporthe_poae | M4FRS1 | QNGCPYPGQSCW |
| Marssonina_brunnea | K1X8D8 | CGYRGQPCP |
| Metarhizium_acridum | E9DXW9 | WCWQPGQPCW |
| Metarhizium_anisopliae | E9EMS3 | WCWRPGQPCW |
| Mycosphaerella_graminicola | F9X131 | GNSFVGWCGAIGAPCA |
| Mycosphaerella_pini | N1Q4Q2 | GVLTRCTVPGLACG |
| Nectria_haematococca | C7ZA34 | WCFYPGQPCW |
| Neosartorya_fischeri | A1D5Z2 | WCHLPGQGCYML |
| Neurospora_crassa | Q1K6I3 | QWCRIHGQSCW |
| Neurospora_tetrasperma | F8MS57 | QWCRIHGQSCW |
| Ogataea_parapolymorpha | W1QE65 | WGWHRVNRNEVIF |
| Ophiostoma_piceae | S3C5N9 | QWCPMVGQPCW |
| Paracoccidioides_lutzii | C1H517 | WCTRPGQGC |
| Penicillium_chrysogenum | B6H2Y5 | WCGHIGQGCY |
| Penicillium_digitatum | K9GDZ2 | WCGHIGQGCY |
| Penicillium_oxalicum | S7Z940 | WCAHPGQGCA |
| Penicillium_roqueforti | W6PVN7 | WCGHIGQGCY |
| Phaeosphaeria_nodorum | Q0UCT8 | YNGWRYRPYGLPVG |
| Pichia_sorbitophila | G8YMJ7 | FHWFKYNKYDPIT |
| Podospora_anserina | B2ADL1 | QWCLRFVGQSCW |
| Pseudogymnoascus_destructans | L8G637 | FCWRPGQPCG |
| Pyrenophora_teres_f_teres | E3RI43 | VTWTQKRPYGMPVG |
| Pyrenophora_tritici-repentis | B2WIP5 | SWTQKRPYGMPVG |
| Saccharomyces_bayanus | Q8J1R6 | WHWLQLKPGQPMY |
| Saccharomyces_castellii | G0VD13 | NWHWLRLDPGQPLY |
| Saccharomyces_cerevisiae | P0CI39 | WHWLQLKPGQPMY |
| Saccharomyces_dairenensis | G0WE84 | WHWLRLDPGQPLY |
| Saccharomyces_mikatae | AACH01001097 | WHWLQLKPGQPMY |
| Saccharomyces_paradoxis | Q8J094 | WHWLQLKPGQPMY |
| Scheffersomyces_stipitis | A3LXU7 | WHWTSYGVFEPG |
| Schizosaccharomyces_japonicus | B6JZE2 | VSDRVKQMLSHWWNFRNPDTANL |
| Schizosaccharomyces_octosporus | S9PVP9 | KTYEDFLRVYKNWWSFQNPDRPDL |
| Schizosaccharomyces_pombe | Q00619 | KTYADFLRAYQSWNTFVNPDRPNL |
| Sclerotinia_borealis | W9C8T9 | WCGRPGQPC |
| Sclerotinia_sclerotiorum | A7EY95 | WCGRPGQPC |
| Sordaria_macrospora | F7W5S1 | QWCRIHGQSCW |
| Sporothrix_schenckii | H9XTI1 | YCPLKGQSCW |
| Tetrapisispora_blattae | I2H305 | HWLRLGRGEPLY |
| Tetrapisispora_phaffii | G8C206 | WHWLRLDPGQPLY |
| Thielavia_heterothallica | G2QGA8 | WCVQFLGMPCW |
| Togninia_minima | R8BGY4 | WCTKHGQSCW |
| Torulaspora_delbrueckii | G8ZR18 | GWMRLRLGQPL |
| Trichoderma_atroviridis | G9NY94 | WCWRVGESCW |
| Trichoderma_jecorina | G0RMK2 | WCYRIGEPCW |
| Trichoderma_virens | G9MQ44 | WCYRVGMTCGW |
| Tuber_melanosporum | D5GJK5 | WTPRPGRGAY |
| Vanderwaltozyma_polyspora_1 | A7TJQ6 | WHWLELDNGQPIY |
| Vanderwaltozyma_polyspora_2 | A7TQX4 | WHWLRLRYGEPIY |
| Vernetllium_alfalfae | C9SGY3 | PCPRPGQGCW |
| Verticillium_dahliae | G2X5W7 | PCPRPGQGCW |
| Wickerhamomyces_ciferrii | K0KPE3 | WQWRKYLNGSPNY |
| Yarrowia_lipolytica | Q6C2Z3 | WRWFWLPGYGEPNW |
| Zygosaccharomyces_bailii | S6EXB4 | HLVRLSPGAAMF |
| Zygosaccharomyces_rouxii | C5DX97 | HFIELDPGQPMF |

In certain embodiments, the secretable GPCR peptide ligand can comprise one or more secretion signal sequences. Non-limiting examples of such secretion signal sequences are provided in Tables 4 and 7. In certain embodiments, the one or more secretion signal sequences are located at the N-terminus of a secretable GPCR peptide ligand. In certain embodiments, a Kex2 processing site and/or a Ste13 processing site or a homolog thereof can be present between the amino acid sequence of the secretion signal sequence and the secretable GPCR peptide ligand.

In certain embodiments, the GPCR ligand, e.g., GPCR peptide ligand, increases the activation of a GPCR disclosed herein from about 1.1 to about 20 fold, e.g., from about 2 to about 20 fold, from about 5 to about 20 fold, from about 10 to about 20 fold, from about 15 to about 20 fold, from about 1.1 to about 15 fold, from about 1.1 to about 10 fold, from about 1.1 to about 5 fold or from about 1.1 to about 2 fold. In certain embodiments, a GPCR ligand, e.g., GPCR peptide ligand, has an EC₅₀ range of, or of about, 1 to 10⁴ nM, e.g., from about 10² nM to about 10³ nM, from about 10² nM to about 10⁴ nM or from about 10³ nM to about 10⁴ nM for a GPCR disclosed herein.

Identification of GPCRs and Ligands

The present disclosure further provides methods for mining and characterizing GPCRs, e.g., fungal GPCRs, and their genetically encoded peptide ligands, e.g., using genomic data as input.

In certain embodiments, an alpha-factor-like GPCR peptide ligand and its cognate GPCR can be identified in scientific literature and databases identifiable by skilled persons such as NCBI, Genbank, Interpro, PFAM or Uniprot, and/or using a “genome-mining” approach such as described in Examples 1 and 2 of the present disclosure, such as using the method reported by Martin et al.⁶⁶ and/or Miguel Jimenez, Doctoral Thesis, Columbia University 2016, and subsequently tested for the ability of an identified GPCR peptide ligand to bind to and activate a GPCR described herein.

In certain embodiments, GPCRs can be identified by searching protein and genomic databases for proteins and/or genes with homology (structural or sequence homology) to known GPCRs, e.g., GPCRs disclosed herein. In certain embodiments, the protein and/or genomic database to be searched is selected from the group consisting of NCBI, Genbank, Interpro, PFAM, Uniprot and a combination thereof.

In certain embodiments, GPCRs can be identified by searching protein and genomic databases for proteins and/or genes with homology (structural or sequence homology) to the S. cerevisiae Ste2 receptor and/or Ste3 receptor. In certain embodiments, the genome-mined GPCRs have an amino acid sequence homology of at least about 15%, e.g., from about 17% to about 68% homology, to S. cerevisiae Ste2 or a motif of Ste2.

In certain embodiments, GPCRs can be identified by searching protein and genomic databases for proteins and/or genes that have conserved regions that is at least about 15%, e.g., from about 17% to about 68%, homologous to the core seven transmembrane helix domain of the S. cerevisiae Ste2 receptor, e.g., Y17 to N301 or one or more of its constituent transmembrane helices, or one of its constituent intracellular signaling loops and associated transmembrane helices, e.g., the amino acid residues spanning from the fifth to the sixth transmembrane helix.

In certain embodiments, GPCRs can be identified by searching protein and genomic databases for proteins and/or genes with homology (structural or sequence homology) to a GPCR disclosed herein. For example, but not by way of limitation, GPCRs can be identified by searching protein and genomic databases for proteins and/or genes with homology (structural or sequence homology) to a GPCR comprising an amino acid sequence comprising any one of SEQ ID NOs: 117-161, a GPCR comprising an amino acid sequence provided in Table 11 and/or a GPCR encoded by a nucleotide sequence comprising any one of SEQ ID NOs: 168-211. In certain embodiments, the genome-mined GPCRs have an amino acid sequence homology of at least about 15%, e.g., from about 17% to about 68% homology, to the GPCR comprising an amino acid sequence comprising any one of SEQ ID NOs: 117-161 and/or the GPCR comprising an amino acid sequence provided in Table 11. In certain embodiments, the genome-mined GPCRs show an amino acid sequence homology of at least about 15%, e.g., from about 17% to about 68% homology, to the GPCR encoded by a nucleotide sequence comprising any one of SEQ ID NOs: 168-211.

The present disclosure provides a method for the identification of a G-protein coupled receptor (GPCR) to be expressed in a genetically-engineered cell. For example, but not by way of limitation, the method can include searching a protein and/or genomic database for a protein and/or a gene with homology to S. cerevisiae Ste2 receptor and/or Ste3 receptor. In certain embodiments, the identified GPCR has an amino acid sequence that is at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to the S. cerevisiae Ste2 receptor and/or Ste3 receptor or a motif thereof. In certain embodiments, the identified GPCR has an amino acid sequence that is at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to the core seven transmembrane helix domain of the S. cerevisiae Ste2 receptor, e.g., Y17 to N301 or one or more of its constituent transmembrane helices, or one of its constituent intracellular signaling loops and associated transmembrane helices, e.g., the amino acid residues spanning from the fifth to the sixth transmembrane helix.

The present disclosure further provides a method for the identification of a GPCR to be expressed in a genetically-engineered cell. For example, but not by way of limitation, the method can include searching a protein and/or genomic database for a protein and/or a gene with homology to a GPCR disclosed herein. In certain embodiments, the identified GPCR has an amino acid sequence that is at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a GPCR comprising an amino acid sequence comprising any one of SEQ ID NOs: 117-161 and/or a GPCR comprising an amino acid sequence provided in Table 11. In certain embodiments, the identified GPCR has a nucleotide sequence that is at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a GPCR encoded by a nucleotide sequence comprising any one of SEQ ID NOs: 168-211.

In certain embodiments, the genome-mined GPCRs have an amino acid sequence having greater than about 15% homology, e.g., greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 91%, greater than about 92%, greater than about 93%, greater than about 94%, greater than about 95%, greater than about 96%, greater than about 97%, greater than about 98% or greater than about 99% homology, to any one of the GPCRs disclosed herein and further comprise a characteristic seven transmembrane helix domain. For example, but not by way of limitation, a genome-mined GPCR of the present disclosure comprises an amino acid sequence that has greater than about 15% homology to an amino acid sequence comprising any one of SEQ ID NOs: 117-161 and/or a GPCR comprising an amino acid sequence provided in Table 11 and further comprises a characteristic seven transmembrane helix domain. In certain embodiments, a genome-mined GPCR of the present disclosure comprises a nucleotide sequence that has greater than about 15% homology to a nucleotide sequence comprising any one of SEQ ID NOs: 168-211 and further comprises a characteristic seven transmembrane helix domain.

In certain embodiments, GPCR ligands can be identified by searching protein and genomic databases for proteins, peptides and/or genes with homology (structural or sequence homology) to known GPCR ligands, e.g., GPCR ligands disclosed herein or pheromone genes, e.g., of yeast (e.g., S. cerevisiae). For example, but not by way of limitation, the identified GPCR ligand has an amino acid sequence that is at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a GPCR ligand that has an amino acid sequence comprising any one of SEQ ID NOs: 1-116, a GPCR ligand that has an amino acid sequence provided a Table 12 or a fungal pheromone. In certain embodiments, the identified GPCR ligand has a nucleotide sequence that is at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a nucleotide sequence comprising any one of SEQ ID NOs: 215-230.

Alternatively and/or additionally, GPCR ligands can be identified from genomes of fungal species by identifying genes, proteins and/or peptides that include regions that are homologous to the processing motifs present in the known pheromone genes, as disclosed herein. For example, pheromone genes have a signature architecture that consists of a hydrophobic prepro secretion signal followed by repeats of the putative secreted peptide flanked by proteolitic processing sites, which can be used to identify GPCR ligands that also include such architecture. In particular, the repetitive nature of the pheromone genes enables prediction of active peptides that bind and induce the corresponding GPCR. For example, but not by way of limitation, putative GPCR ligands can be identified by the presence of flanking processing sites such as X-A and X-P dipeptides and/or Kex2-like cleavage sites (KR, QR, NR) that appear between each repeated region (i.e., the repeated region excluding the processing site is the active GPCR ligand). In certain embodiments, identified GPCR ligand genes, protein and/or peptides include flanking processing sites, e.g., often with a single site preceding a short C-terminal peptide that is the active ligand.

In certain embodiments, the genome-mined GPCR ligands have an amino acid sequence that has greater than about 15% homology, e.g., greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 91%, greater than about 92%, greater than about 93%, greater than about 94%, greater than about 95%, greater than about 96%, greater than about 97%, greater than about 98% or greater than about 99% homology, to any one of the GPCR peptide ligands disclosed herein and further comprise a characteristic pre-pro motif and/or one or more processing sites. For example, but not by way of limitation, a genome-mined GPCR peptide of the present disclosure comprises an amino acid sequence that has greater than about 15% homology to an amino acid sequence comprising any one of SEQ ID NOs: 1-116 and/or a GPCR peptide ligand comprising an amino acid sequence provided in Table 12, and further comprises a characteristic pre-pro motif and/or one or more processing sites. In certain embodiments, a genome-mined GPCR peptide ligand of the present disclosure comprises a nucleotide sequence that has greater than about 15% homology to a nucleotide sequence comprising any one of SEQ ID NOs: 215-230, and further comprises a characteristic pre-pro motif and/or one or more processing sites.

In certain embodiments, GPCR ligands can be identified by searching for proteins and/or peptides (or genes that encode such proteins and/or peptides) that have certain conserved features such as, but not limited to, aromatic amino acids at the termini, e.g., tryptophan at the N-terminus, and/or paired cysteines near the termini.

In certain embodiments, a variant GPCR or a variant GPCR ligand can be obtained using a method of directed evolution. The term “directed evolution” means a process wherein random mutagenesis is applied to a protein (e.g., a GPCR or a GPCR peptide ligand), and a selection regime is used to pick out variants that have the desired qualities, such as selecting for an altered binding and/or activation. Accordingly, polynucleotides encoding a GPCR or a GPCR ligand as described herein (e.g., in the Examples) can be genetically mutated using recombinant techniques known to those of ordinary skill in the art, including by site-directed mutagenesis, or by random mutagenesis such as by exposure to chemical mutagens or to radiation, as known in the art. An advantage of directed evolution is that it requires no prior structural knowledge of a protein, nor is it necessary to be able to predict what effect a given mutation will have. In general, in the intercellular signaling system of the present disclosure that includes at least two cells, a first cell is adapted to secrete a peptide configured to activate a GPCR of a second cell as described herein. Because GPCRs couple well to the conserved yeast MAP-kinase signaling cascade³⁶, the fungal mating peptide/GPCR-based intercellular signaling system described herein overcomes limitations of previous intercellular signaling systems and can be harnessed as a source of modular parts for engineering a scalable intercellular signaling system. For example, but not by way of limitation, the GPCRs, disclosed herein, can undergo directed evolution to alter it specificity to a certain ligand, e.g., to increase its binding to a ligand and/or decrease its binding to a ligand.

In certain embodiments, a variant GPCR or a variant GPCR ligand can be obtained using family shuffling to generate new GPCRs that have altered ligand-binding properties. The term “family shuffling” means a process where DNA fragments of a family of related GPCRs are randomly recombined to generate variant GPCRs that are selected for the desired qualities, such as selecting for an altered binding and/or activation. See, e.g., Kikuchi and Harayama (2002) DNA Shuffling and Family Shuffling for In Vitro Gene Evolution. In: Braman J. (eds) In Vitro Mutagenesis Protocols. Methods in Molecular Biology, Vol. 182; and Meyer et al., Library Generation by Gene Shuffling, Curr. Protoc. Mol. Biol. (2014) 105:15.12.1-15.12.7, which are incorporated by reference herein in their entireties.

### III. Cells

Cells for use in the intercellular signaling systems of the present disclosure can be cells, e.g., genetically-engineered cells, that express a heterologous GPCR and/or secrete a GPCR ligand. For example, but not by way of limitation, a cell for use in the present disclosure can express one or more GPCR ligands, disclosed herein. In certain embodiments, a cell for use in the present disclosure can express one or more heterologous GPCRs, disclosed herein.

In certain embodiments, the cell for use in the intercellular signaling systems of the present disclosure can be a mammalian cell, a plant cell or a fungal cell. For example, but not by way of limitation, the cell can be a mammalian cell, e.g., a genetically-engineered mammalian cell. In certain embodiments, the cell can be a plant cell, e.g., a genetically-engineered plant cell.

In certain embodiments, the cell can be a fungal cell, e.g., a genetically-engineered fungal cell. For example, but not by way of limitation, the cell can be a cell of the phylum Ascomycota. In certain embodiments, the cells, e.g., two or more cells, of intercellular signaling systems of the present disclosure are cells independently selected from any species of the phylum Ascomycota. In certain embodiments, the cells can be species independently selected from Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, and Capronia coronata.

In certain embodiments, two or more cells of an intercellular signaling system (e.g., all the cells of an intercellular signaling system) can be of the same species of the phylum Ascomycota or cell type. For example, but not by way of limitation, two or more cells (or all the cells) can be Saccharomyces cerevisiae. Alternatively, at least one of the cells within an intercellular signaling system is of a different species of the phylum Ascomycota or cell type.

In certain embodiments, one or more endogenous GPCR genes of the cells and/or one or more endogenous GPCR peptide ligand genes of the cells are knocked out.

For example, but not by way of limitation, the one or more knocked out endogenous GPCR genes can comprise an STE2 gene and/or an STE3 gene. In certain embodiments, one or more of the knocked out endogenous GPCR peptide ligand genes can comprise an MFA1/2 gene, an MFALPHA1/MFALPHA2 gene, a BAR1 gene and/or an SST2 gene. In certain embodiments, the FAR1 gene can be knocked out. In certain embodiments, a cell for use in the present disclosure has one or more, two or more, three or more, four or more, five or more, six or more or all seven of following genes knocked out: STE2, STE3, MFA1/2, MFALPHA1/MFALPHA2, BAR1, SST2 and FAR1.

In certain embodiments, a genetic engineering system is employed to knock out the genes disclosed herein, e.g., one or more endogenous GPCR genes and/or one or more endogenous GPCR peptide ligand genes, in a cell. Various genetic engineering systems known in the art can be used for the methods disclosed herein. Non-limiting examples of such systems include the Clustered regularly-interspaced short palindromic repeats (CRISPR)/Cas system, the zinc-finger nuclease (ZFN) system, the transcription activator-like effector nuclease (TALEN) system, use of yeast endogenous homologous recombination and the use of interfering RNAs.

In certain non-limiting embodiments, a CRISPR/Cas9 system is employed to knock out the one or more endogenous GPCR genes and/or one or more endogenous GPCR peptide ligand genes in a cell. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9) and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule, and referred to alternatively as chimeric) or modular (comprising more than one, and typically two, separate RNA molecules, such as a crRNA and a tracrRNA, which are usually associated with one another, for instance by duplexing).

In certain embodiments, the CRISPR/Cas9 system comprises a Cas9 molecule and one or more gRNAs, e.g., 2 gRNAs, comprising a targeting domain that is complementary to a target sequence of one or more endogenous GPCR genes and/or one or more endogenous GPCR peptide ligand genes. For example, but not by way of limitation, the target sequence can be a sequence within a GPCR peptide ligand gene, e.g., a MFA1/2 gene, a MFALPHA1/MFALPHA2 gene, a BAR1 gene and/or an SST2 gene. In certain embodiments, the target sequence is a sequence within a GPCR peptide ligand gene, e.g., an STE2 gene and/or an STE3 gene. In certain embodiments, the target sequence can be a 5′ region flanking the open reading frame of the gene to be knocked out and/or a 3′ region flanking the open reading frame of the gene to be knocked out. For example, but not by way of limitation, a CRISPR/Cas9 system for use in the present disclosure comprises a Cas9 molecule and two gRNAs, where one gRNA targets a 5′ region flanking the open reading frame of the gene to be knocked out and the second gRNA targets a 3′ intron region flanking the open reading frame of the gene to be knocked out. Non-limiting examples of gRNAs are disclosed in Table 8. For example, but not by way of limitation, a gRNA for use in knocking out one or more endogenous GPCR genes and/or one or more endogenous GPCR peptide ligand genes comprises a nucleotide sequence set forth in any one of SEQ ID NOs: 231-253.

In certain embodiments, the gRNAs are administered to the cell in a single vector and the Cas9 molecule is administered to the cell in a second vector. In certain embodiments, the gRNAs and the Cas9 molecule are administered to the cell in a single vector. Alternatively, each of the gRNAs and Cas9 molecule can be administered by separate vectors. In certain embodiments, the CRISPR/Cas9 system can be delivered to the cell as a ribonucleoprotein complex (RNP) that comprises a Cas9 protein complexed with one or more gRNAs, e.g., delivered by electroporation (see, e.g., DeWitt et al., Methods 121-122:9-15 (2017) for additional methods of delivering RNPs to a cell).

In certain embodiments, the two or more cells of the intercellular communication system has a mating type selected from a MA Ta-type and a MA Ta-type.

The cells to be used in the present disclosure can be genetically-engineered using recombinant techniques known to those of ordinary skill in the art. Production and manipulation of the polynucleotides described herein are within the skill in the art and can be carried out according to recombinant techniques described, for example, in Sambrook et al. 1989. Molecular Cloning: A Laboratory Manual, 2d ed., Cold Spring Harbor Laboratory Press, Cold Spring Harbor, N.Y. and Innis et al. (eds). 1995. PCR Strategies, Academic Press, Inc., San Diego.

### IV. Intercellular Signaling Systems

The present disclosure provides intercellular signaling systems that comprise at least two cells that can communicate with one another and methods of promoting intercellular signaling between at least two cells. For example, but not by way of limitation, an intercellular signaling system of the present disclosure includes at least two or more, at least three or more, at least four or more, at least five or more, at least six or more, at least seven or more, at least eight or more, at least nine or more, at least ten or more, at least fifteen or more, at least twenty or more, at least thirty or more, at least forty or more or at least fifty or more cells that can communicate with one another.

In certain embodiments, at least one of the cells (e.g., each of the cells) of the intercellular signaling system expresses a heterologous GPCR. In certain embodiments, at least one of the cells of the intercellular signaling system express more than one heterologous GPCR. For example, but not by way of limitation, one or more cells of the intercellular signaling system can express one, two, three, four, five or more heterologous GPCRs, e.g., where each GPCR binds to and are activated by different ligands. In certain embodiments, the heterologous GPCRs are encoded by a nucleic acid that is present within the cell, e.g., the cells comprise a nucleic acid that encodes at least one heterologous GPCR. The GPCR can be heterologous by virtue of having its origin in another type of organism, e.g., a different species of fungus, and/or being a variant and/or derivative of a native GPCR in the same or different type of organism, e.g., a product of directed evolution. Non-limiting examples of GPCRs that can be encoded by the nucleic acid are disclosed herein.

In certain embodiments, at least one of the cells (e.g., each of the cells) of the intercellular signaling system expresses a ligand, e.g., a GPCR ligand. In certain embodiments, at least one of the cells of the intercellular signaling system express more than one ligand. For example, but not by way of limitation, one or more cells of the intercellular signaling system can express one, two, three, four, five or more ligands, e.g., where each ligand binds to and activate different GPCRs. In certain embodiments, the ligand, e.g., a protein or peptide ligand, is encoded by a nucleic acid that is present within the cell, e.g., the cells comprise a nucleic acid that encodes at least one ligand. In certain embodiments, each cell of the intercellular signaling system includes a nucleic acid that encodes a secretable ligand, e.g., a secretable protein or a secretable peptide. In certain embodiments, the nucleic acid encodes a peptide, e.g., a secretable GPCR peptide ligand. For example, but not by way of limitation, activation of a GPCR expressed by a cell results in the expression and secretion of the secretable GPCR peptide ligand from the cell, e.g., by signaling through a G-protein signaling pathway. The secretable GPCR peptide ligand can, in turn, bind to and activate a second GPCR on a separate cell within the intercellular signaling system. Non-limiting examples of secretable GPCR peptide ligands that can be encoded by the nucleic acid are disclosed herein.

In certain embodiments, one or more cells of the intercellular signaling pathway can include a nucleic acid encoding an essential gene. An “essential gene,” as used herein, refers to a gene that when expressed in a cell is required for the growth and/or survival of the cell, e.g., under any growth condition. Non-limiting examples of essential genes include PKC1, RPB11 and SEC4. Additional non-limiting examples of essential genes in yeast are disclosed in Kofed et al., G3 (Bethesda) 5(9):1879-1887 (2015). For example, but not by way of limitation, the essential gene can be SEC4.

In certain embodiments, one or more cells of the intercellular signaling pathway can include a nucleic acid encoding a conditionally essential gene. A “conditionally essential gene,” as used herein, refers to a gene that is essential for growth and/or survival under certain conditions but not others, e.g., in the absence of an essential media component. In certain embodiments, a conditionally essential gene can be a gene that is required to generate an essential amino acid. Non-limiting examples of conditionally essential genes include HIS3 and TRP1.

In certain embodiments, one or more cells of the intercellular signaling pathway can include a nucleic acid encoding a toxic gene. A “toxic gene,” as used herein, refers to a gene that results in the death of a cell under certain conditions, e.g., where the gene encodes a protein that coverts a compound present in the media into a toxic compound. A non-limiting example of a toxic gene include URA3. For example, but not by way of limitation, URA3 encodes a protein that converts 5-fluoroorotic acid (5-FOA) present in the media to 5-fluorouracil, which is toxic.

In certain embodiments, such essential genes, conditionally essential genes and toxic genes can be used to engineer mutually-dependent communities, where one or more cells within a community rely on or are suppressed by the expression and secretion of a GPCR peptide ligand from other distinct cells within the same community.

In certain embodiments, one or more cells of the intercellular signaling pathway can include a nucleic acid encoding a product of interest. Non-limiting examples of such products of interest include hormones, toxins, receptors, fusion proteins, regulatory factors, growth factors, complement system factors, clotting factors, anti-clotting factors, kinases, cytokines, CD proteins, interleukins, therapeutic proteins, diagnostic proteins, enzymes, biosynthetic pathways, antibiotics and antibodies.

In certain embodiments, one or more cells of the intercellular signaling system can include a nucleic acid that encodes a detectable reporter. For example, but not by way of limitation, a detectable reporter includes a label, e.g., a compound capable of emitting a detectable signal, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image (e.g., as seen for fluorescent reporters in the Examples). In certain embodiments, the term “labeling signal” as used herein indicates the signal emitted from the label that allows detection of the label, including but not limited to radioactivity, fluorescence, chemiluminescence, production of a compound in outcome of an enzymatic reaction (e.g., production of colored compounds) and the like.

The detection of the reporter can be performed by various methods identifiable by those skilled in the art, such as in vitro methods: fluorescence, absorbance, mass spectrometry, flow cytometry colorimetric, visual, UV, gas chromatography, liquid chromatography, an electronic output, activation of ion channels, protein gels, Western blot, thin layer chromatography and radioactivity. In particular a labeling signal can be quantitative or qualitatively detected with these techniques as will be understood by a skilled person. For example, but not by way of limitation, a fluorescent protein such as GFP can be detected with an excitation range of 485 and an emission range of 515, and mRFP can be detected with an excitation range of 580 and an emission range of 610. Other fluorescent proteins include without limitation sfGFP, deGFP, eGFP, Venus, YFP, Cerulean, Citrine, CFP, eYFP, eCFP, mRFP, mCherry, mmCherry. Other reportable molecular components do not require excitation to be detected; for example, colorimetric reportable molecular components can have a detectable color without fluorescent excitation. Other detectable signals include dyes that can be bound to genetic molecular components and then released upon an activity (e.g., sequestration, FRET, digestion).

In certain embodiments, one or more cells of the intercellular signaling system can include a nucleic acid that encodes a sensor, e.g., a protein (e.g., a receptor such as a GPCR), that detects one or more analytes or agents of interest that differ from the ligands that interact with the heterologous GPCR expressed by the cell. Non-limiting examples of such analytes or agents of interest include heavy metals, metabolites, small molecules and light. Additional non-limiting examples of such analytes or agents of interest include human disease agents (human pathogenic agents), agricultural agents, industrial/model organism agents and bioterrorism agents. See U.S. Publication No. 2017/0336407, the contents of which are disclosed by reference herein in its entirety.

In certain embodiments, an intercellular signaling system of the present disclosure includes a cell, e.g., a genetically-engineered cell, that expresses at least one heterologous GPCR. In certain embodiments, the heterologous GPCR is encoded by a nucleic acid that is present within the cell. For example, but not by way of limitation, an intercellular signaling system of the present disclosure includes a cell that comprises at least one nucleic acid encoding a heterologous GPCR present within the cell. In certain embodiments, the GPCR is activated by an exogenously supplied ligand. Non-limiting examples of ligands, e.g., a synthetic ligand, that can activate a GPCR are described herein.

In certain embodiments, an intercellular signaling system of the present disclosure includes a cell, e.g., a genetically-engineered cell, that expresses at least one secretable GPCR ligand, e.g., a GPCR peptide ligand. In certain embodiments, the secretable GPCR ligand is encoded by nucleic acid that are present within the cell. For example, but not by way of limitation, an intercellular signaling system of the present disclosure includes a cell that comprises at least one nucleic acid that encodes a secretable GPCR ligand, e.g., a GPCR peptide ligand. In certain embodiments, the expression of the secretable GPCR ligand can be activated by a ligand-inducible promoter. In certain embodiments, the expression of the secretable GPCR ligand can be induced by the activation of an endogenous GPCR or a heterologous GPCR that results in the expression of the secretable GPCR ligand.

In certain embodiments, an intercellular signaling system of the present disclosure includes a cell, e.g., a genetically-engineered cell, that expresses at least one heterologous GPCR and at least one secretable GPCR ligand, e.g., a GPCR peptide ligand. In certain embodiments, the secretable GPCR ligand expressed by the genetically-engineered cell does not activate the heterologous GPCR of the same cell. In certain embodiments, the secretable GPCR ligand expressed by the genetically-engineered cell selectively interacts with and activates the heterologous GPCR of the same cell. In certain embodiments, the heterologous GPCRs and secretable GPCR ligand are encoded by nucleic acids that are present within the cells. For example, but not by way of limitation, an intercellular signaling system of the present disclosure includes at least one cell, where the cell includes at least one nucleic acid encoding a first GPCR and at least one nucleic acid that encodes a first secretable GPCR ligand, e.g., a GPCR peptide ligand. In certain embodiments, the secretable GPCR peptide ligand that is secreted from the cell selectively interacts with and activates the heterologous GPCR expressed by the cell. Alternatively, the secretable GPCR peptide ligand that is secreted from the cell does not activate the heterologous GPCR expressed by the cell.

In certain embodiments, an intercellular signaling system of the present disclosure includes two or more cells, where the first cell expresses at least one secretable GPCR ligand, e.g., a GPCR peptide ligand, and the second cell expresses at least one heterologous GPCR. In certain embodiments, the GPCR ligand secreted by the first cell selectively interacts with and activates the heterologous GPCR expressed by the second cell. In certain embodiments, the heterologous GPCRs and secretable GPCR ligand are encoded by nucleic acids that are present within the cells. For example, but not by way of limitation, an intercellular signaling system of the present disclosure includes two or more cells, where one cell includes at least one nucleic acid that encodes a first secretable GPCR ligand, e.g., a GPCR peptide ligand, and the second cell includes at least one nucleic acid encoding a second GPCR. In certain embodiments, the first secretable GPCR peptide ligand that is secreted from the first cell selectively interacts with and activates the second GPCR expressed by the second cell. In certain embodiments, the first cell can further express a heterologous GPCR (e.g., different from the heterologous GPCR expressed by the second cell and/or which is not activated by the secretable GPCR ligand expressed by the first cell) and the second cell can further express a secretable GPCR ligand (e.g., that is different from the secretable GPCR ligand expressed by the first cell and/or does not activate the heterologous GPCR expressed by the second cell).

In certain embodiments, an intercellular signaling system of the present disclosure includes two or more cells, where the first cell expresses at least one heterologous GPCR and at least one secretable GPCR ligand, e.g., a GPCR peptide ligand, and the second cell expresses at least one heterologous GPCR. In certain embodiments, the heterologous GPCR expressed by the second cell is different from the heterologous GPCR expressed by the first cell, e.g., are selectively activated by different ligands. In certain embodiments, the GPCR ligand secreted by the first cell selectively interacts with and activates the heterologous GPCR expressed by the second cell. In certain embodiments, the heterologous GPCRs and secretable GPCR ligand are encoded by nucleic acids that are present within the cells. For example, but not by way of limitation, an intercellular signaling system of the present disclosure includes two or more cells, where one cell includes at least one nucleic acid encoding a first GPCR and at least one nucleic acid that encodes a first secretable GPCR ligand, e.g., a GPCR peptide ligand, and the second cell includes at least one nucleic acid encoding a second GPCR. In certain embodiments, the first secretable GPCR peptide ligand that is secreted from the first cell selectively interacts with and activates the second GPCR expressed by the second cell. In certain embodiments, the first cell is the same cell as the second cell.

In certain embodiments, an intercellular signaling system of the present disclosure includes two or more cells, where a first cell expresses a first heterologous GPCR and a first secretable GPCR ligand, e.g., a first GPCR peptide ligand, and a second cell expresses a second heterologous GPCR and a second secretable GPCR ligand, e.g., a second GPCR peptide ligand. In certain embodiments, the heterologous GPCRs and secretable GPCR ligands are encoded by nucleic acids that are present within the cells. For example, but not by way of limitation, an intercellular signaling system of the present disclosure includes two or more cells, where one cell includes at least one nucleic acid encoding a first GPCR and at least one nucleic acid that encodes a first secretable GPCR ligand, e.g., a GPCR peptide ligand, and the second cell includes at least one nucleic acid encoding a second GPCR and at least one nucleic acid that encodes a second secretable GPCR ligand, e.g., a GPCR peptide ligand.

In certain embodiments, the first heterologous GPCR and the second heterologous GPCR have sequence homologies of less than about 30% and/or the first secretable GPCR ligand and the second secretable GPCR ligand have sequence homologies of less than about 40%, e.g., to generate an orthogonal intercellular signaling system. For example, but not by way of limitation, an intercellular signaling system of the present disclosure can include (i) a first genetically-engineered cell that expresses a first heterologous GPCR and/or a first secretable GPCR peptide ligand and (ii) a second cell expresses a second heterologous GPCR and/or a second secretable GPCR peptide ligand, wherein the first heterologous GPCR and the second heterologous GPCR have sequence homologies of less than about 30%, e.g., from about 1% to about 29% or from about 0% to about 29%, and/or the first secretable GPCR peptide ligand and the second secretable GPCR peptide ligand have sequence homologies of less than about 40%, e.g., from about 1% to about 39% or from about 0% to about 39%.

In certain embodiments, the first secretable GPCR peptide ligand that is secreted from the first cell selectively interacts with and activates the second GPCR expressed by the second cell. In certain embodiments, the second secretable GPCR peptide ligand that is secreted from the second cell selectively interacts with and activates the first GPCR expressed by the second cell. Alternatively, the second secretable GPCR peptide ligand that is secreted from the second cell does not interact with and activate the first GPCR expressed by the second cell.

In certain embodiments, an intercellular signaling system of the present disclosure can include a third cell, where the third cell expresses a third heterologous GPCR and/or a third GPCR ligand. For example, but not by way of limitation, the third cell can include at least one nucleic acid encoding a third GPCR and/or at least one nucleic acid that encodes a third secretable GPCR ligand, e.g., a GPCR peptide ligand. For example, but not by way of limitation, the second secretable GPCR peptide ligand that is secreted from the second cell selectively interacts with and activates the third GPCR expressed by the third cell. For example, but not by way of limitation, an intercellular signaling system of the present disclosure can include a third cell, where the third cell includes at least one nucleic acid encoding a third GPCR and at least one nucleic acid that encodes a third secretable GPCR ligand, e.g., a GPCR peptide ligand. For example, but not by way of limitation, the second secretable GPCR peptide ligand that is secreted from the second cell selectively interacts with and activates the third GPCR expressed by the third cell. Alternatively and/or additionally, the first secretable GPCR peptide ligand that is secreted from the first cell selectively interacts with and activates the third GPCR expressed by the third cell.

In certain embodiments, an intercellular signaling system of the present disclosure can include a fourth cell (or fifth, sixth or seventh, etc. cell) where the fourth cell (or fifth, sixth or seventh, etc. cell) includes a nucleic acid encoding a fourth (or fifth, sixth or seventh, etc.) GPCR and/or a nucleic acid that encodes a fourth (or fifth, sixth or seventh, etc.) secretable GPCR ligand, e.g., GPCR peptide ligand. For example, but not by way of limitation, the third secretable GPCR peptide ligand that is secreted from the third cell selectively interacts with and activates the fourth GPCR expressed by the fourth cell. In certain embodiments, two or more cells of an intercellular signaling system disclosed herein can express the same secretable GPCR ligand that selectively interacts with and activates a GPCR expressed by one or more cells within the system. Alternatively and/or additionally, one or more cells of an intercellular signaling system disclosed herein can express a secretable GPCR ligand that selectively interacts with and activates a GPCR that is expressed by two or more cells within the system.

In certain embodiments, the intercellular signaling system networks described herein can have a daisy chain network topology. For example, but not by way of limitation, in each intermediate cell of the network, the GPCR peptide ligand secreted from a cell that immediately precedes the intermediate cell in the topology of the intercellular signaling system network is different from the secretable GPCR peptide ligand secreted from the intermediate cell. In addition, the GPCR expressed by the intermediate cell is different from the GPCR expressed by a cell that immediately precedes the intermediate cell and expressed by a cell that immediately follows the intermediate cell. The terms “precedes” and “follows” refer to the cell-to-cell flow of an intercellular signal through the network topology. In certain embodiments, a daisy chain network topology can be a daisy chain linear network topology or a daisy chain ring network topology. In certain embodiments, a daisy chain linear network topology or a daisy chain ring network topology can further comprise one or more branches that extend from one or more intermediary cells in the network topology.

In certain embodiments, the intercellular signaling system networks described herein can have a star network topology. For example, but not by way of limitation, a “star” type of network comprises branches, e.g., a cell or cells, that can be connected to each other through a singular common link, e.g., cell.

In certain embodiments, the intercellular signaling system networks described herein can have a bus topology. For example, but not by way of limitation, a “bus” type of network comprises cells that can be connected to each other through a singular common link, e.g., cell.

In certain embodiments, the intercellular signaling system networks described herein can have a branched topology. For example, but not by way of limitation, a “branched” type of network comprises one or more branches, e.g., a cell or cells, that extend from one or more intermediary cells.

In certain embodiments, the intercellular signaling system networks described herein can have a ring topology. For example, but not by way of limitation, a “ring” type of network comprises cells that are connected in a manner where the last cell in the chain is connected back to the first cell in the chain.

In certain embodiments, the intercellular signaling system networks described herein can have mesh topology. For example, but not by way of limitation, a “mesh” type of network is a network where all the cells with the network are connected to as many other cells as possible.

In certain embodiments, the intercellular signaling system networks described herein can have a hybrid topology. For example, but not by way of limitation, a “hybrid” type of network is a network that includes a combination of two or more topologies.

In certain embodiments, a network of can include one or more of these network subtypes, e.g., a branched type network, a bus type network, a ring network, a mesh network, a hybrid network, a star type network and/or a daisy chain network, joined by one or more nodes, e.g., cells. See, for example, FIG. 25.

In certain embodiments, a cell can include one or more nucleic acids encoding one or more heterologous GPCRs, e.g., two or more, three or more or four or more nucleic acids to encode two or more, three or more or four or more heterologous GPCRs. Alternatively or additionally, a single nucleic acid can encode more than one heterologous GPCR, e.g., two or more, three or more or four or more heterologous GPCRs. In certain embodiments, a cell can include one or more nucleic acids encoding one or more secretable GPCR ligands, e.g., two or more, three or more or four or more nucleic acids to encode two or more, three or more or four or more secretable GPCR ligands. Alternatively and/or additionally, a single nucleic acid can encode more than one secretable GPCR ligand, e.g., two or more, three or more or four or more secretable GPCR ligands.

In certain embodiments, nucleic acids of the present disclosure can be introduced into the cells of the intercellular communication system using vectors, such as plasmid vectors, and cell transformation techniques such as electroporation, heat shock and others known to those skilled in the art and described herein. In certain embodiments, the genetic molecular components are introduced into the cell to persist as a plasmid or integrate into the genome. In certain embodiments, the cells can be engineered to chromosomally integrate a polynucleotide of one or more genetic molecular components described herein, using methods identifiable to skilled persons upon reading the present disclosure.

In certain embodiments, a nucleic acid encoding a GPCR or a secretable GPCR ligand is introduced into the yeast cell either as a construct or a plasmid. In certain embodiments, a nucleic acid encoding a GPCR or a secretable GPCR peptide ligand can comprise one or more regulatory regions such as promoters, transcription factor binding sites, operators, activator binding sites, repressor binding sites, enhancers, protein-protein binding domains, RNA binding domains, DNA binding domains, and other control elements known to a person skilled in the art. For example, but not by way of limitation, a nucleic acid encoding a GPCR or a secretable GPCR peptide ligand is introduced into the yeast cell either as a construct or a plasmid in which it is operably linked to a promoter active in the yeast cell or such that it is inserted into the yeast cell genome at a location where it is operably linked to a suitable promoter.

Non-limiting examples of suitable yeast promoters include, but are not limited to, constitutive promoters pTef1, pPgk1, pCyc1, pAdh1, pKex1, pTdh3, pTpi1, pPyk1 and pHxt7 and inducible promoters pGal1, pCup1, pMet15, pFig1 and pFus1. For example, but not by way of limitation, a nucleic acid encoding the GPCR can include a constitutively active promoter, e.g., pTdh3. In certain embodiments, a nucleic acid encoding the secretable GPCR peptide ligand can include an inducible promoter, e.g., pFus1 or pFig1. In certain embodiments, a nucleic acid encoding the secretable GPCR peptide ligand can include a constitutively active promoter, e.g., pAdh1.

In certain embodiments, a nucleic acid encoding a GPCR or a secretable GPCR ligand can be inserted into the genome of the cell, e.g., yeast cell. For example, but not by way of limitation, one or more nucleic acids encoding a GPCR or a secretable GPCR ligand can be inserted into the Ste2, Ste3 and/or HO locus of the cell. In certain embodiments, the one or more nucleic acids can be inserted into one or more loci that minimally affects the cell, e.g., in an intergenic locus or a gene that is not essential and/or does not affect growth, proliferation and cell signaling.

### V. Methods of Use

The present disclosure further provides methods for using the intercellular signaling systems described herein.

In certain embodiments, the intercellular signaling systems described herein are useful for applications such as synthetic biology, computing, biomanufacturing of biofuels, pharmaceuticals or food additives using yeast, biological sensors, biomaterials, logic gates, switches, screening platform for drug development and toxicology, precision diagnostics tools, model systems to study cell signaling and for artificial plant, animal and human tissues, secretion of peptide and/or protein therapeutics, secretion of small molecule therapeutics, among others.

In certain embodiments, the intercellular signaling systems of the present disclosure can be used for the generation of pharmaceuticals and/or therapeutics. For example, but not by way of limitation, the intercellular signaling systems of the present disclosure can be used for the generation of pharmaceuticals and/or therapeutics that require the assembly of multiple components in a coordinated manner, where each cell of the intercellular signaling system is configured to produce a component of the pharmaceutical. For example, but not by way of limitation, such methods can include the use of a intercellular signaling system that includes a first cell (or a first group of cells), e.g., a yeast cell, that senses a target of interest and communicates with a second cell (or a second group of cells), e.g., a yeast cell, (e.g., by secretion of a ligand that binds to a GPCR expressed by the second cell) where the second cell (or second group of cells) secretes a therapeutic of interest or an intermediate of the therapeutic of interest, e.g., an antibiotic or an intermediate of the antibiotic. Alternatively and/or additionally, such methods can include a intercellular signaling system that includes a network in which a first cell (or a first group of cells), e.g., a yeast cell, senses a target of interest and communicates with second cell (or a second group of cells), e.g., a yeast cell, to analyze the sensed data and in which a third cell (or a third group of cells) cell, e.g., a yeast cell, secretes a therapeutic of interest (or an intermediate of the therapeutic of interest) in response to the sensed target of interest. In certain embodiments, the target of interest can include a marker, indicator and/or biomarker of a disorder and/or disease.

In certain embodiments, a method for the production of a pharmaceutical and/or therapeutic includes providing an intercellular signaling system disclosed herein. For example, but not by way of limitation, an intercellularly signaling system for use in methods for the production of a pharmaceutical and/or therapeutic can include two cells, e.g., two genetically-engineered cells, e.g., two genetically-engineered yeast strains. In certain embodiments, the first cell, e.g., the first genetically modified cell, of the intercellular signaling system, expresses a GPCR, e.g., a heterologous GPCR, that can be activated by a target of interest, e.g., an indicator, biomarker and/or marker of a particular disease or disorder. Upon detection of the target of interest, the first genetically modified cell expresses a secretable GPCR ligand that can selectively activate a heterologous GPCR expressed by the second cell, e.g., second genetically modified cell. Upon activation of the heterologous GPCR expressed by the second cell, the second cell produces a product of interest, e.g., a pharmaceutical and/or a therapeutic. For example, but not by way of limitation, the first genetically modified cell expresses a GPCR, e.g., a heterologous GPCR, that can be activated by different levels of glucose. Upon detection of certain levels of glucose, the first genetically modified cell expresses a secretable GPCR ligand (e.g., the amount of GPCR ligand produced can depend on the level of glucose detected) that can selectively activate the heterologous GPCR expressed by the second cell, e.g., second genetically modified cell. Upon activation of the heterologous GPCR expressed by the second cell, the second cell produces and secretes different insulin levels depending on the level of glucose detected.

In certain embodiments, the intercellular signaling systems of the present disclosure can be used for spatial control of gene expression and/or temporal control of gene expression.

In certain embodiments, the intercellular signaling systems of the present disclosure can be used for generating biomaterials.

In certain embodiments, the intercellular signaling systems of the present disclosure can be used for biosensing. For example, but not by way of limitation, one or more cells of an intercellular signaling system herein can express a receptor (e.g., a GPCR) or other sensing/responsive module (e.g., by introducing a nucleic acid encoding the receptor or sensing/responsive module) that is responsive, e.g., can bind to, one or more agents (molecules) of interest. Non-limiting examples of agents of interest include human disease agents (human pathogenic agents), agricultural agents, industrial and model organism agents, bioterrorism agents and heavy metal contaminants. Human disease agents include, but are not limited to, infectious disease agents, oncological disease agents, neurodegenerative disease agents, kidney disease agents, cardiovascular disease agents, clinical chemistry assay agents, and allergen and toxin agents. Additional non-limiting examples of such agents of interest include hormones, sugars, peptides, metals, metalloids, lipids, biomarkers and combinations thereof. Further non-limiting examples of agents of interests and GPCRs for use in detecting such agents of interest, are disclosed in U.S. Publication No. 2017/0336407, the contents of which are disclosed by reference herein in its entirety.

In certain embodiments, the sensing of an agent of interest by one or more cells of an intercellular signaling system can result in the production and/or secretion of a product of interest by other cells within the intercellular signaling system. For example, but not by way of limitation, the product of interest can be a hormone, toxin, receptor, fusion protein, regulatory factor, growth factor, complement system factor, enzyme, clotting factor, anti-clotting factor, kinase, cytokine, CD protein, interleukins, therapeutic protein, diagnostic protein, biosynthetic pathway and antibody. Such intercellular signaling systems can produce a product of interest in response to an agent of interest. This sense-and-respond behavior can be modulated by building any type of network topology referenced herein (e.g., bus, daisy chain, etc.). In certain embodiments, the sense-and-respond behavior can be tuned such that specific input concentrations lead to desired output concentrations. In certain embodiments, a first cell (or first group of cells) of an intercellular signaling pathway can include a nucleic acid that encodes a receptor or other sensing/responsive module responsive to an agent of interest and include a second cell (or second group of cells) within the same intercellular signaling pathway can include a nucleic acid encoding a product of interest. For example, but not by way of limitation, an intercellular signaling system for use in biosensing can include (i) a first cell that (a) expresses a heterologous GPCR that binds an agent of interest and (b) expresses a secretable GPCR ligand upon binding the agent of interest; and (ii) a second cell that (a) expresses a heterologous GPCR that binds to the secretable GPCR ligand expressed by the first cell and (b) expresses a product of interest. In certain embodiments, the agent of interest is a human disease agent and the product of interest is a therapeutic for treating the human disease caused by the human disease agent.

In certain embodiments, the intercellular signaling systems of the present disclosure can be used for performing computations. Non-limiting examples of such computations include mathematical equations, logic gates and computational algorithms. In certain embodiments, an intercellular signaling system for performing computations can include a network in which different cells, e.g., yeast cells (e.g., genetically-engineered yeast cells), perform computation and where the information flow is done by the sensing (e.g., binding) and secretion of peptides and proteins by the different cells of the system. In certain embodiments, an intercellular signaling system having any type of network topology, as disclosed herein, can be utilized to perform computations, e.g., mathematical equations, logic gates and computational algorithms, where the cells of the system can sense one or more inputs, process the information and give one or more outputs. In certain embodiments, equations and algorithms can be used to predict and optimize the setup of any type of network in order to achieve desired input-output processing outcomes.

### VI. Kits

The present disclosure further provides kits to generate the intercellular signaling systems described herein. For example, a kit of the present disclosure can include one or more cells, one or more GPCR-encoding nucleic acids, one or more GPCR ligand-encoding nucleic acids, one or more essential gene-encoding nucleic acids and/or one or more nucleic acids that encode a product of interest disclosed herein.

In certain embodiments, a kit of the present disclosure can include a first container comprising at least one or more genetically-engineered cells disclosed herein. In certain embodiments, the genetically-engineered cell expresses a heterologous GPCR, e.g., encoded by a nucleic acid. In certain embodiments, the genetically-engineered cell expresses a GPCR ligand, e.g., encoded by a nucleic acid.

In certain embodiments, the first genetically-engineered cell includes (i) a nucleic acid encoding a heterologous G-protein coupled receptor (GPCR); and/or (ii) a nucleic acid encoding a secretable GPCR ligand. In certain embodiments, the kit can further comprise a second container that includes a second genetically-engineered cell comprising: (i) a nucleic acid encoding a heterologous GPCR; and/or (ii) a nucleic acid encoding a secretable GPCR ligand. In certain embodiments, the GPCR of the first and/or second cell is at least about 75% homologous to an amino acid sequence comprising any one of SEQ ID NOs: 117-161 and/or is encoded by a nucleotide sequence that is at least about 75% homologous to a nucleotide sequence comprising any one of SEQ ID NOs: 168-211. In certain embodiments, the heterologous GPCR of the first genetically-engineered cell is different than the heterologous GPCR of the second genetically-engineered cell, e.g., bind to different ligands. In certain embodiments, the secretable GPCR ligand of the first genetically-engineered cell is different than the secretable GPCR ligand of the second genetically-engineered cell, e.g., bind to different GPCRs.

Alternatively and/or additionally, a kit of the present disclosure can include one or more containers that include one or more components of an intercellular signaling system described herein. For example, but not by way of limitation, one or more containers can include one or more nucleic acids, e.g., vectors, that encode a heterologous GPCR and/or a secretable GPCR ligand.

### VII. Exemplary Embodiments

A. The presently disclosed subject matter provides a genetically-engineered cell expressing at least one heterologous G-protein coupled receptor (GPCR), wherein the amino acid sequence of the heterologous GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211.

A1. The foregoing genetically-engineered cell, wherein the amino acid sequence of the heterologous GPCR is at least about 95% homologous to an amino acid sequence comprising any one of SEQ ID NOs: 117-161 or an amino acid sequence provided in Table 11 and/or is encoded by a nucleotide sequence that is at least about 95% homologous to a nucleotide sequence comprising any one of SEQ ID NOs: 168-211.

A2. The foregoing genetically-engineered cell of A and A1, wherein the heterologous GPCR is selectively activated by a ligand.

A3. The foregoing genetically-engineered cell of A2, wherein the ligand is selected from the group consisting of peptide, a protein or portion thereof, a small molecule, a nucleotide, a lipid, a chemical, a photon, an electrical signal and a compound.

A4. The foregoing genetically-engineered cell of A3, wherein the ligand is a compound.

A5. The foregoing genetically-engineered cell of A3, wherein the ligand is a protein or portion thereof.

A6. The foregoing genetically-engineered cell of A3, wherein the ligand is a peptide.

A7. The foregoing genetically-engineered cell of A6, wherein the peptide comprises about 3 to about 50 amino acid residues.

A8. The genetically-engineered cell of A6 or A7, wherein the amino acid sequence of the peptide is at least about 75% homologous to an amino acid sequence comprising any one of SEQ ID NOs: 1-72 or an amino acid sequence provided in Table 12.

A9. The foregoing genetically-engineered cell of any one of A6-A8, wherein the amino acid sequence of the peptide is at least about 95% homologous to an amino acid sequence comprising any one of SEQ ID NOs: 73-116 or an amino acid sequence provided in Table 12.

A10. The foregoing genetically-engineered cell of any one of A6-A9, wherein the peptide is encoded by a nucleotide sequence that is about 75% homologous to a nucleotide sequence comprising any one of SEQ ID NOs: 215-230.

A11. The foregoing genetically-engineered cell of any one of A-A10, wherein the cell further expresses at least one secretable GPCR ligand.

A12. The foregoing genetically-engineered cell of A11, wherein the at least one secretable GPCR ligand is a peptide or a protein or portion thereof.

A13. The foregoing genetically-engineered cell of A12, wherein the secretable GPCR ligand is a peptide.

A14. The foregoing genetically-engineered cell of A13, wherein the peptide comprises about 3 to about 50 amino acid residues.

A15. The foregoing genetically-engineered cell of any one of A11-A14, wherein the secretable GPCR ligand is identified and/or derived from a eukaryotic organism.

A16. The foregoing genetically-engineered cell of A15, wherein the eukaryotic organism is selected from the group consisting of an animal, plant, fungus and/or protozoan.

B. The presently disclosure provides a genetically-engineered cell expressing at least one heterologous secretable G-protein coupled receptor (GPCR) peptide ligand, wherein the amino acid sequence of the peptide is at least about 75% homologous to an amino acid sequence comprising any one of SEQ ID NOs: 1-72 or an amino acid sequence provided in Table 12.

B1. The foregoing genetically-engineered cell of B, wherein the amino acid sequence of the peptide is at least about 95% homologous to an amino acid sequence comprising any one of SEQ ID NOs: 73-116 or an amino acid sequence provided in Table 12.

B2. The foregoing genetically-engineered cell of B or B1, wherein the peptide is encoded by a nucleotide sequence that is about 75% homologous to a nucleotide sequence comprising any one of SEQ ID NOs: 215-230.

B3. The foregoing genetically-engineered cell of any one of B-B2, wherein the cell further expresses at least one heterologous G-protein coupled receptor (GPCR).

B4. The foregoing genetically-engineered cell of B3, wherein the heterologous GPCR is identified and/or derived from a eukaryotic organism.

B5. The foregoing genetically-engineered cell of B4, wherein the eukaryotic organism is selected from the group consisting of an animal, plant, fungus and/or protozoan.

B6. The foregoing genetically-engineered cell of any one of A-A16 and B-B5, wherein the genetically-engineered cell is selected from the group consisting of a mammalian cell, a plant cell and a fungal cell.

B7. The foregoing genetically-engineered cell of B6, wherein the genetically-engineered cell is a fungal cell.

B8. The foregoing genetically-engineered cell of B7, wherein the fungal cell is a species of the phylum Ascomycota.

B9. The foregoing genetically-engineered cell of B8, wherein the species of the phylum Ascomycota is selected from the group consisting of Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, Capronia coronate and combinations thereof.

C. The present disclosure further provides an intercellular signaling system comprising one or more genetically-engineered cells of any one of A-A16 and B-B9.

C1. The foregoing intercellular signaling system of C, wherein the heterologous GPCR is activated by an exogenous ligand.

C2. The foregoing intercellular signaling system of C1, wherein the exogenous ligand is selected from the group consisting of a peptide, a protein or portion thereof, a small molecule, a nucleotide, a lipid, chemicals, a photon, an electrical signal and a compound.

C3. The foregoing intercellular signaling system of C2, wherein the exogenous ligand is a peptide.

D. The presently disclosed subject matter provides for an intercellular signaling system comprising: (a) a first genetically-engineered cell expressing at least one secretable G-protein coupled receptor (GPCR) ligand; and (b) a second genetically-engineered cell expressing at least one heterologous GPCR, wherein the amino acid sequence of the at least one heterologous GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211, wherein the secretable GPCR ligand of the first genetically-engineered cell selectively activates the heterologous GPCR of the second genetically-engineered cell.

D1. The foregoing intercellular signaling system of D, wherein the amino acid sequence of the at least one heterologous GPCR is at least about 95% homologous to an amino acid sequence comprising any one of SEQ ID NOs: 117-161 or an amino acid sequence provided in Table 11 and/or is encoded by a nucleotide sequence that is at least about 95% homologous to a nucleotide sequence comprising any one of SEQ ID NOs: 168-211.

D2. The foregoing intercellular signaling system of any one of D or D1, wherein the secretable GPCR ligand is identified and/or derived from a eukaryotic organism.

D3. The foregoing intercellular signaling system of D2, wherein the eukaryotic organism is selected from the group consisting of an animal, plant, fungus and/or protozoan.

D4. The foregoing intercellular signaling system of any one of D-D3, wherein the secretable GPCR ligand is selected from the group consisting of a protein or portion thereof and a peptide.

D5. The foregoing intercellular signaling system of D4, wherein the secretable GPCR ligand is a protein or portion thereof.

D6. The foregoing intercellular signaling system of D4, wherein the secretable GPCR ligand is a peptide.

D7. The foregoing intercellular signaling system of D6, wherein the peptide comprises about 3 to about 50 amino acid residues.

D8. The foregoing intercellular signaling system of D6 or D7, wherein the peptide is at least about 75% homologous to an amino acid sequence comprising any one of SEQ ID NOs: 1-72 or an amino acid sequence provided in Table 12.

D9. The foregoing intercellular signaling system of any one of D6-D8, wherein the peptide is at least about 95% homologous to an amino acid sequence comprising any one of SEQ ID NOs: 73-116 or an amino acid sequence provided in Table 12.

D10. The foregoing intercellular signaling system of any one of D6-D9, wherein the peptide is encoded by a nucleotide sequence that is about 75% homologous to a nucleotide sequence comprising any one of SEQ ID NOs: 215-230.

E. The present disclosure further provides an intercellular signaling system comprising: (a) a first genetically-engineered cell expressing at least one secretable G-protein coupled receptor (GPCR) peptide ligand; and (b) a second genetically-engineered cell expressing at least one heterologous GPCR, wherein the amino acid sequence of the secretable GPCR peptide ligand is at least about 75% homologous to an amino acid sequence comprising any one of SEQ ID NOs: 1-72 or an amino acid sequence provided in Table 12 and/or is encoded by a nucleotide sequence that is about 75% homologous to a nucleotide sequence comprising any one of SEQ ID NOs: 215-230, wherein the secretable GPCR ligand of the first genetically-engineered cell selectively activates the heterologous GPCR of the second genetically-engineered cell.

E1. The foregoing intercellular signaling system of E, wherein the heterologous GPCR is identified and/or derived from a eukaryotic organism.

E2. The foregoing intercellular signaling system of E1, wherein the eukaryotic organism is selected from the group consisting of an animal, plant, fungus and/or protozoan.

E3. The foregoing intercellular signaling system of any one of D-D10 and E-E2, wherein the second genetically-engineered cell further expresses at least one secretable GPCR ligand, and wherein the secretable GPCR ligand expressed by the second genetically-engineered cell is different from the secretable GPCR ligand expressed by the first genetically-engineered cell, e.g., selectively activate different GPCRs.

E4. The foregoing intercellular signaling system of any one of D-D10 and E-E3, wherein the first genetically-engineered cell further expresses at least one heterologous GPCR, wherein the heterologous GPCR expressed by the first genetically-engineered cell is different from the heterologous GPCR expressed by the second genetically-engineered cell, e.g., are selectively activated by different ligands.

E5. The foregoing intercellular signaling system of E3 or E4, wherein the secretable GPCR ligand expressed by the second genetically-engineered cell does not activate the heterologous GPCR expressed by the second genetically-engineered cell and/or does not activate the heterologous GPCR expressed by the first genetically-engineered cell.

E6. The foregoing intercellular signaling system of E5, wherein the secretable GPCR ligand expressed by the second genetically-engineered cell does not activate the heterologous GPCR expressed by the second genetically-engineered cell and activates the heterologous GPCR expressed by the first genetically-engineered cell.

F. The present disclosure provides an intercellular signaling system comprising: (a) a first genetically-engineered cell expressing at least one heterologous G-protein coupled receptor (GPCR); and (b) a second genetically-engineered cell expressing at least one secretable GPCR ligand, wherein the amino acid sequence of the at least one heterologous GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211, wherein the secretable GPCR ligand of the second genetically-engineered cell does not activate the heterologous GPCR of the first genetically-engineered cell.

F1. The foregoing intercellular signaling system of F, wherein the amino acid sequence of the at least one heterologous GPCR is at least about 95% homologous to an amino acid sequence comprising any one of SEQ ID NOs: 117-161 or an amino acid sequence provided in Table 11 and/or is encoded by a nucleotide sequence that is at least about 95% homologous to a nucleotide sequence comprising any one of SEQ ID NOs: 168-211.

F2. The foregoing intercellular signaling system of any one of F or F1, wherein the secretable GPCR ligand is identified and/or derived from a eukaryotic organism.

F3. The foregoing intercellular signaling system of F2, wherein the eukaryotic organism is selected from the group consisting of an animal, plant, fungus and/or protozoan.

F4. The foregoing intercellular signaling system of any one of F-F3, wherein the secretable GPCR ligand is selected from the group consisting of a protein or portion thereof and a peptide.

F5. The foregoing intercellular signaling system of F4, wherein the secretable GPCR ligand is a protein or portion thereof.

F6. The foregoing intercellular signaling system of F4, wherein the secretable GPCR ligand is a peptide.

F7. The foregoing intercellular signaling system of F6, wherein the peptide comprises about 3 to about 50 amino acid residues.

F8. The foregoing intercellular signaling system of any one of F6 or F7, wherein the peptide is at least about 75% homologous to an amino acid sequence comprising any one of SEQ ID NOs: 1-72 or an amino acid sequence provided in Table 12.

F9. The foregoing intercellular signaling system of any one of F6-F8, wherein the peptide is at least about 95% homologous to an amino acid sequence comprising any one of SEQ ID NOs: 73-116 or an amino acid sequence provided in Table 12.

F10. The foregoing intercellular signaling system of any one of F6-F8, wherein the peptide is encoded by a nucleotide sequence that is about 75% homologous to a nucleotide sequence comprising any one of SEQ ID NOs: 215-230.

G. The present disclosure further provides an intercellular signaling system comprising: (a) a first genetically-engineered cell expressing at least one heterologous G-protein coupled receptor (GPCR); and (b) a second genetically-engineered cell expressing at least one secretable GPCR peptide ligand, wherein the amino acid sequence of the secretable GPCR peptide ligand is at least about 75% homologous to an amino acid sequence comprising any one of SEQ ID NOs: 1-72 or an amino acid sequence provided in Table 12 and/or is encoded by a nucleotide sequence that is about 75% homologous to a nucleotide sequence comprising any one of SEQ ID NOs: 215-230, wherein the secretable GPCR ligand of the second genetically-engineered cell does not activate the heterologous GPCR of the first genetically-engineered cell.

G1. The foregoing intercellular signaling system of G, wherein the heterologous GPCR is identified and/or derived from a eukaryotic organism.

G2. The foregoing intercellular signaling system of G1, wherein the eukaryotic organism is selected from the group consisting of an animal, plant, fungus and/or protozoan.

G3. The foregoing intercellular signaling system of any one of F-F10 and G-G2, wherein the heterologous GPCR is activated by an exogenous ligand.

G4. The foregoing intercellular signaling system of G3, wherein the exogenous ligand is selected from the group consisting of a peptide, a protein or portion thereof, a small molecule, a nucleotide, a lipid, chemicals, a photon, an electrical signal and a compound.

G5. The foregoing intercellular signaling system of G4, wherein the exogenous ligand is a peptide.

G6. The foregoing intercellular signaling system of any one of F-F10 and G-G5, wherein the first genetically-engineered cell further expresses at least one secretable GPCR ligand, and wherein the secretable GPCR ligand expressed by the second genetically-engineered cell is different from the secretable GPCR ligand expressed by the first genetically-engineered cell, e.g., selectively activate different GPCRs.

G7. The foregoing intercellular signaling system of any one of F-F10 and G-G6, wherein the second genetically-engineered cell further expresses at least one heterologous GPCR, wherein the heterologous GPCR expressed by the first genetically-engineered cell is different from the heterologous GPCR expressed by the second genetically-engineered cell, e.g., are selectively activated by different ligands.

G8. The foregoing intercellular signaling system of any one of F-F10 and G-G7, wherein the first genetically-engineered cell and the second genetically-engineered cell are cells independently selected from the group consisting of mammalian cells, plant cells, fungal cells and combinations thereof.

G9. The foregoing intercellular signaling system of G8, wherein the first genetically-engineered cell and the second genetically-engineered cell are fungal cells.

G10. The foregoing intercellular signaling system of G9, wherein the first genetically-engineered cell and the second genetically-engineered cell are fungal cells independently selected from any species of the phylum Ascomycota.

G11. The foregoing intercellular signaling system of G10, wherein the first genetically-engineered cell and the second genetically-engineered cell are independently selected from the group consisting of Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, Capronia coronate and combinations thereof.

G12. The foregoing intercellular signaling system of any one of D-D10, E-E6, F-F10 and G-G11, wherein the at least one heterologous GPCR expressed by the first genetically-engineered cell and/or second genetically-engineered cell is encoded by a nucleic acid.

G13. The foregoing intercellular signaling system of any one of D-D10, E-E6, F-F10 and G-G12, wherein the at least one secretable GPCR ligand expressed by the first genetically-engineered cell and/or second genetically-engineered cell is encoded by a nucleic acid.

G14. The foregoing intercellular signaling system of any one of C-C3, D-D10, E-E6, F-F10 and G-G13, wherein one or more endogenous GPCR genes of the one or more genetically-engineered cells, the first genetically-engineered cell and/or the second genetically-engineered cell are knocked out.

G15. The foregoing intercellular signaling system of G14, wherein the one or more endogenous GPCR genes comprises an STE2 gene and/or an STE3 gene.

G16. The intercellular signaling system of any one of C-C3, D-D10, E-E6, F-F10 and G-G15, wherein one or more endogenous GPCR ligand genes of the one or more genetically-engineered cells, the first genetically-engineered cell and/or the second genetically-engineered cell are knocked out.

G17. The foregoing intercellular signaling system of G16, wherein the one or more of the endogenous GPCR ligand genes comprises an MFA1/2 gene, an MFALPHA1/MFALPHA2 gene, a BAR1 gene and/or an SST2 gene.

G18. The foregoing intercellular signaling system of any one of G14-G17, wherein a genetic engineering system is used to knock out the one or more endogenous GPCR genes and/or the one or more endogenous GPCR ligand genes.

G19. The foregoing intercellular signaling system of G18, wherein the genetic engineering system is selected from the group consisting of a CRISPR/Cas system, a zinc-finger nuclease (ZFN) system, a transcription activator-like effector nuclease (TALEN) system and interfering RNAs.

G20. The foregoing intercellular signaling system of G19, wherein the genetic engineering system is a CRISPR/Cas system.

G21. The foregoing intercellular signaling system of any one of C-C3, D-D10, E-E6, F-F10 and G-G20, wherein the one or more genetically-engineered cells, the first genetically-engineered cell and/or the second genetically-engineered cell further comprises a nucleic acid encoding an essential gene, a conditionally essential gene and/or a toxic gene.

G22. The foregoing intercellular signaling system of any one of C-C3, D-D10, E-E6, F-F10 and G-G21, wherein the one or more genetically-engineered cells, the first genetically-engineered cell and/or the second genetically-engineered cell further comprises a nucleic acid encoding an essential gene, a conditionally essential gene and/or a toxic gene.

G23. The foregoing intercellular signaling system of any one of C-C3, D-D10, E-E6, F-F10 and G-G22, wherein the one or more genetically-engineered cells, the first genetically-engineered cell and/or the second genetically-engineered cell further comprises a nucleic acid that encodes a product of interest.

G24. The foregoing intercellular signaling system of G23, wherein the product of interest is selected from the group consisting of hormones, toxins, receptors, fusion proteins, regulatory factors, growth factors, complement system factors, enzymes, clotting factors, anti-clotting factors, kinases, cytokines, CD proteins, interleukins, therapeutic proteins, diagnostic proteins, enzymes, antibiotics, biosynthetic pathways, antibodies and combinations thereof.

G25. The foregoing intercellular signaling system of any one of C-C3, D-D10, E-E6, F-F10 and G-G24, wherein the one or more genetically-engineered cells, the first genetically-engineered cell and/or the second genetically-engineered cell further comprises a nucleic acid that encodes a detectable reporter.

G26. The foregoing intercellular signaling system of any one of C-C3, D-D10, E-E6, F-F10 and G-G25, wherein the one or more genetically-engineered cells, the first genetically-engineered cell and/or the second genetically-engineered cell further comprises a nucleic acid that encodes a sensor.

G27. The foregoing intercellular signaling system of any one of D-D10, E-E6, F-F10 and G-G26 further comprising a third genetically-engineered cell, a fourth genetically-engineered cell, a fifth genetically-engineered cell, a sixth genetically-engineered cell, a seventh genetically-engineered cell, an eighth genetically-engineered cell or more, wherein each of the genetically-engineered cells expresses at least one heterologous GPCR and/or at least one secretable GPCR ligand, wherein each of the heterologous GPCRs are different, e.g., are selectively activated by different ligands, and/or each of the secretable GPCR ligands are different, e.g., selectively activate different GPCRs.

G28. The foregoing intercellular signaling system of G27, wherein (i) the secretable ligand expressed by the second cell selectively activates the GPCR expressed by the third cell; (ii) the secretable ligand expressed by the third cell selectively activates the GPCR expressed by the fourth cell; (iii) the secretable ligand expressed by the fourth cell selectively activates the GPCR expressed by the fifth cell; (iv) the secretable ligand expressed by the fifth cell selectively activates the GPCR expressed by the sixth cell; (v) the secretable ligand expressed by the sixth cell selectively activates the GPCR expressed by the seventh cell; and/or (vi) the secretable ligand expressed by the seventh cell selectively activates the GPCR expressed by the eight cell.

G29. The foregoing intercellular signaling system of G27, wherein the intercellular signaling system comprises a daisy chain network topology.

G30. The foregoing intercellular signaling system of G27, wherein the intercellular signaling system comprises a bus type network topology.

G31. The foregoing intercellular signaling system of G27, wherein the intercellular signaling system comprises a branched type network topology.

G32. The foregoing intercellular signaling system of G27, wherein the intercellular signaling system comprises a star type network topology.

G33. The foregoing intercellular signaling system of G27, wherein the intercellular signaling system comprises a daisy chain network topology, a bus type network topology, a branched type network topology, a ring network topology, a mesh network topology, a hybrid network topology, a star type network topology or a combination thereof.

H. The present disclosure further provides an intercellular signaling system comprising a first genetically-engineered cell comprising a nucleic acid encoding at least one first heterologous G-protein coupled receptor (GPCR), wherein the first heterologous GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211.

H1. The foregoing intercellular signaling system of H, wherein the amino acid sequence of the heterologous GPCR is at least about 95% homologous to an amino acid sequence comprising any one of SEQ ID NOs: 117-161 or an amino acid sequence provided in Table 11 and/or is encoded by a nucleotide sequence that is at least about 95% homologous to a nucleotide sequence comprising any one of SEQ ID NOs: 168-211.

H2. The foregoing intercellular signaling system of H or H1, wherein the heterologous GPCR is selectively activated by a ligand.

H3. The foregoing intercellular signaling system of H2, wherein the ligand is selected from the group consisting of peptide, a protein or portion thereof, a small molecule, a nucleotide, a lipid, a chemical, a photon, an electrical signal and a compound.

H4. The foregoing intercellular signaling system of H3, wherein the ligand is a compound.

H5. The foregoing intercellular signaling system of H3, wherein the ligand is a protein or portion thereof.

H6. The foregoing intercellular signaling system of H3, wherein the ligand is a peptide.

H7. The foregoing intercellular signaling system of H6, wherein the peptide comprises about 3 to about 50 amino acid residues.

H8. The foregoing intercellular signaling system of any one of H-H7, wherein the first genetically-engineered cell further comprises a nucleic acid encoding a first heterologous secretable GPCR ligand.

H9. The foregoing intercellular signaling system of H8, wherein the secretable GPCR ligand is identified and/or derived from a eukaryotic organism.

H10. The foregoing intercellular signaling system of H9, wherein the eukaryotic organism is selected from the group consisting of an animal, plant, fungus and/or protozoan.

I. The present disclosure provides an intercellular signaling system comprising a first genetically-engineered cell comprising a nucleic acid encoding at least one first secretable G-protein coupled receptor (GPCR) peptide ligand, wherein the amino acid sequence of the secretable GPCR peptide ligand is at least about 75% homologous to an amino acid sequence comprising any one of SEQ ID NOs: 1-72 or an amino acid sequence provided in Table 12.

I1. The foregoing intercellular signaling system of I, wherein the amino acid sequence of the secretable GPCR peptide ligand is at least about 95% homologous to an amino acid sequence comprising any one of SEQ ID NOs: 73-116 or an amino acid sequence provided in Table 12.

I2. The foregoing intercellular signaling system of I, wherein the secretable GPCR peptide ligand is encoded by a nucleotide sequence that is about 75% homologous to a nucleotide sequence comprising any one of SEQ ID NOs: 215-230.

I3. The foregoing intercellular signaling system of any one of I-I2, wherein the cell further comprises a nucleic acid that encodes at least one heterologous G-protein coupled receptor (GPCR).

I4. The foregoing intercellular signaling system of I3, wherein the heterologous GPCR ligand is identified and/or derived from a eukaryotic organism.

I5. The foregoing intercellular signaling system of I4, wherein the eukaryotic organism is selected from the group consisting of an animal, plant, fungus and/or protozoan.

I6. The foregoing intercellular signaling system of any one of H-H10 and I-I5, wherein the genetically-engineered cell is selected from the group consisting of a mammalian cell, a plant cell and a fungal cell.

I7. The foregoing intercellular signaling system of I6, wherein the genetically-engineered cell is a fungal cell.

I8. The foregoing intercellular signaling system of I7, wherein the fungal cell is a species of the phylum Ascomycota.

I9. The foregoing intercellular signaling system of I8, wherein the species of the phylum Ascomycota is selected from the group consisting of Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, Capronia coronate and combinations thereof.

I10. The foregoing intercellular signaling system of any one of H-H10 and I-19 further comprising a second genetically-engineered cell.

I11. The foregoing intercellular signaling system of I10, wherein the second genetically-engineered cell comprises a nucleic acid encoding a second heterologous secretable GPCR ligand.

I12. The foregoing intercellular signaling system of I10 or I11, wherein the second genetically-engineered cell comprises a nucleic acid encoding a second heterologous GPCR.

I13. The foregoing intercellular signaling system of I12, wherein the first heterologous secretable ligand selectively activates the second heterologous GPCR.

J. The present disclosure provides an intercellular signaling system comprising: (a) a first genetically-engineered cell comprising: (i) a nucleic acid encoding a first heterologous G-protein coupled receptor (GPCR); and/or (ii) a nucleic acid encoding a first secretable GPCR ligand; and (b) a second genetically-engineered cell comprising: (i) a nucleic acid encoding a second heterologous GPCR; and/or (ii) a nucleic acid encoding a second secretable GPCR ligand, wherein the first GPCR and/or the second GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211, and/or wherein the first and/or second secretable GPCR peptide ligand is at least about 75% homologous to an amino acid sequence comprising any one of SEQ ID NOs: 1-72 or an amino acid sequence provided in Table 12 and/or is encoded by a nucleotide sequence that is about 75% homologous to a nucleotide sequence comprising any one of SEQ ID NOs: 215-230.

J1. The foregoing intercellular signaling system of J, wherein the first secretable GPCR ligand of the first genetically-engineered cell selectively activates the second heterologous GPCR of the second genetically-engineered cell.

J2. The foregoing intercellular signaling system of J, wherein the second secretable GPCR ligand of the second genetically-engineered cell selectively activates the first heterologous GPCR of the first genetically-engineered cell.

J3. The foregoing intercellular signaling system of J, wherein the second secretable GPCR ligand of the second genetically-engineered cell selectively does not activate the first heterologous GPCR of the first genetically-engineered cell.

J4. The foregoing intercellular signaling system of any one of J-J3, wherein the first GPCR and the second GPCR are selectively activated by different ligands.

J5. The foregoing intercellular signaling system of any one of J-J4 further comprising a third genetically-engineered cell, wherein the third genetically-engineered cell comprises: (i) a nucleic acid encoding a third heterologous GPCR; and/or (ii) a nucleic acid encoding a third secretable GPCR ligand.

J6. The foregoing intercellular signaling system of J5, wherein the second secretable GPCR ligand of the second genetically-engineered cell selectively activates the third heterologous GPCR of the third genetically-engineered cell.

J7. The foregoing intercellular signaling system of J5 or J6, wherein the first secretable GPCR ligand of the first genetically-engineered cell selectively activates the third heterologous GPCR of the third genetically-engineered cell.

K. The present disclosure provides a kit comprising a genetically-modified cell of any one of A-A16 and B-B9.

L. The present disclosure further provides kit comprising an intercellular signaling system of any one of C-C3, D-D10, E-E6, F-F10, G-G33, H-H10, I-I13 and J-J7.

M. The present disclosure provides a method of using an intercellular signaling system of any one of C-C3, D-D10, E-E6, F-F10, G-G33, H-H10, I-I13 and J-J7 for the generation of pharmaceuticals.

N. The present disclosure provides a method of using an intercellular signaling system of any one of C-C3, D-D10, E-E6, F-F10, G-G33, H-H10, I-I13 and J-J7 for spatial control of gene expression and/or temporal control of gene expression.

O. The present disclosure provides a method of using an intercellular signaling system of any one of C-C3, D-D10, E-E6, F-F10, G-G33, H-H10, I-I13 and J-J7 for the generation of product of interest.

P. The present disclosure provides a method for the identification of a G-protein coupled receptor (GPCR) to be expressed in a genetically-engineered cell, comprising searching a protein and/or genomic database and/or literature for a protein and/or a gene with homology to S. cerevisiae Ste2 receptor and/or Ste3 receptor.

P1. The foregoing method of P, wherein the identified GPCR has an amino acid sequence that is at least about 15% homologous to the S. cerevisiae Ste2 receptor and/or Ste3 receptor.

Q. The present disclosure provides a method for the identification of a G-protein coupled receptor (GPCR) to be expressed in a genetically-engineered cell, comprising searching a protein and/or genomic database and/or literature for a protein and/or a gene with homology to (a) a GPCR comprising an amino acid sequence comprising any one of SEQ ID NOs: 117-161; (b) a GPCR comprising an amino acid sequence provided in Table 11; and/or (c) a GPCR encoded by a nucleotide sequence comprising any one of SEQ ID NOs: 168-211.

Q1. The method of Q, wherein the identified GPCR has an amino acid sequence that is at least about 15% homologous to the GPCR comprising an amino acid sequence comprising any one of SEQ ID NOs: 117-161 and/or the GPCR comprising an amino acid sequence provided in Table 11.

Q2. The method of Q, wherein the identified GPCR has a nucleotide sequence that is at least 15% homologous to the GPCR encoded by a nucleotide sequence comprising any one of SEQ ID NOs: 168-211.

R. The present disclosure provides a method for the identification of a GPCR ligand to be expressed in a genetically-engineered cell, comprising searching a protein and/or genomic database and/or literature for a protein, peptide and/or a gene with homology to: (i) a GPCR peptide ligand comprising an amino acid sequence comprising any one of SEQ ID NOs: 1-116; (ii) a GPCR peptide ligand comprising an amino acid sequence provided in Table 12; (iii) a GPCR peptide ligand encoded by a nucleotide sequence comprising any one of SEQ ID NOs: 215-230; and/or (iv) a yeast pheromone or a motif thereof.

R1. The method of R, wherein the identified GPCR ligand has an amino acid sequence that is at least about 15% homologous to (i) the GPCR peptide ligand comprising an amino acid sequence comprising any one of SEQ ID NOs: 1-116; (ii) the GPCR peptide ligand comprising an amino acid sequence provided in Table 12; (iii) the GPCR peptide ligand encoded by a nucleotide sequence comprising any one of SEQ ID NOs: 215-230; and/or (iv) the yeast pheromone or a motif thereof.

R2. The method of any one of P-P1, Q-Q2 and R-R1, wherein the protein and/or genomic database is selected from the group consisting of NCBI, Genbank, Interpro, PFAM, Uniprot and a combination thereof.

S. The present disclosure provides a genetically-engineered cell expressing a G-protein coupled receptor (GPCR) and/or a GPCR ligand identified by the method of any one of P-P1, Q-Q2 and R-R2.

### EXAMPLES

The following examples are put forth so as to provide those of ordinary skill in the art with a complete disclosure and description of how to make and use the presently disclosed subject matter and are not intended to limit the scope of what the inventors regard as their presently disclosed subject matter. It is understood that various other implementations and embodiments can be practiced, given the general description provided herein.

### Example 1. Methods

The following methods were used in the Examples disclosed herein.

Strains. Yeast strains and the plasmids contained are listed in Table 2. All strains are directly derived from BY4741 (MATα leu2Δ0 met15Δ0 ura3Δ0 his3Δ1) and BY4742 (MATα leu2Δ0 lys2Δ0 ura3Δ0 his3Δ1) by engineered deletion using CRISPR Cas9^{58,59}.

TABLE 2
Strains used in this study. The reference in Table 2 indicated by a superscript
“11” is Brachmann, C. B. et al. Designer deletion strains derived from
Saccharomyces cerevisiae S288C: a useful set of strains and plasmids for
PCR-mediated gene disruption and other applications. Yeast 14, 115-132 (1998).

| Strain | | | |
|---|---|---|---|
| name | Genotype | Comment | Reference |
| BY4741 | MATa leu2Δ0 met15Δ0 ura3Δ0 | Parent of yNA899 | ¹¹ |
| | his3Δ1 | | |
| BY4742 | MATα lys2Δ0 leu2Δ0 ura3Δ0 | Parent of yNA903 | ¹¹ |
| | his3Δ1 | | |
| yNA899 | MATa leu2Δ0 met15Δ0 ura3Δ0 | Parent of JTy014 | This study |
| | his3Δ1 MFa1Δ MFa2Δ | | |
| | MFalpha1Δ MFalpha2Δ ste2Δ | | |
| | ste3Δ sst2Δ far1Δ bar1Δ | | |
| yNA903 | MATα lys2Δ0 leu2Δ0 ura3Δ0 | Used for validation of language | This study |
| | his3Δ1 MFa1Δ MFa2Δ | functionality in α-type strain | |
| | MFalpha1Δ MFalpha2Δ ste2Δ | | |
| | ste3Δ sst2Δ far1Δ bar1Δ | | |
| JTy014 | MATa leu2Δ0 met15Δ0 ura3Δ0 | Used for GPCR characterization | This study |
| | his3Δ1 MFa1Δ MFa2Δ | after transformation with the | |
| | MFalpha1Δ MFalpha2Δ ste2Δ | GPCR expression constructs. | |
| | ste3Δ sst2Δ far1Δ bar1Δ | Parent of ySB98/99/100 | |
| | HO::FUS1p-coRFP-LEU2 | | |
| JTy015 | MATa leu2Δ0 met15Δ0 ura3Δ0 | | This study |
| | his3Δ1 MFa1Δ MFa2Δ | | |
| | MFalpha1Δ MFalpha2Δ ste2Δ | | |
| | ste3Δ sst2Δ far1Δ bar1Δ | | |
| | HO::FIG1p-coRFP-LEU2 | | |
| ySB98 | MATa leu2Δ0 met15Δ0 ura3Δ0 | Ca.Ste2/Sc.Ste2 or Bc.Ste2 under | This study |
| | his3Δ1 MFa1Δ MFa2Δ | control of the constitutive TDH3 | |
| | MFalpha1Δ MFalpha2Δ ste2Δ | promoter integrated into the Ste2 | |
| | ste3Δ sst2Δ far1Δ bar1Δ | locus. Used for single cell analysis | |
| | HO::FUS1p-coRFP-LEU2 | and GPCR activation-deactivation | |
| | ste2::TDH3p-Ca.Ste2-STE2t | experiments | |
| ySB99 | MATa leu2Δ0 met15Δ0 ura3Δ0 | | This study |
| | his3Δ1 MFa1Δ MFa2Δ | | |
| | MFalpha1Δ MFalpha2Δ ste2Δ | | |
| | ste3Δ sst2Δ far1Δ bar1Δ | | |
| | HO::FUS1p-coRFP-LEU2 | | |
| | Ste2::TDH3p-Sc.Ste2-STE2t | | |
| ySB100 | MATa leu2Δ0 met15Δ0 ura3Δ0 | | This study |
| | his3Δ1 MFa1Δ MFa2Δ | | |
| | MFalpha1Δ MFalpha2Δ ste2Δ | | |
| | ste3Δ sst2Δ far1Δ bar1Δ | | |
| | HO::FUS1p-coRFP-LEU2 | | |
| | Ste2::TDH3p-Bc.Ste2-STE2t | | |
| ySB265 | MATa leu2Δ0 met15Δ0 ura3Δ0 | Ste12 replaced by Ste12*. | This study |
| | his3Δ1 MFa1Δ MFa2Δ | TDH3p-Bc.Ste2, Ca.Ste2 or | |
| | MFalpha1Δ MFalpha2Δ ste2Δ | Vp1.Ste2 integrated into the STE2 | |
| | ste3Δ sst2Δ far1Δ bar1Δ | locus. SEC4 under control of | |
| | ste12::ste12* ste2::TDH3p- | OSR1 promoter and insulated by | |
| | Bc.Ste2 sec4::CYC1t-OSR1p- | an upstream CYC1 terminator or | |
| | Sec4 | under control of the OSR4 | |
| ySB270 | MATa leu2Δ0 met15Δ0 ura3Δ0 | promoter without insulation. Used | This study |
| | his3Δ1 MFa1Δ MFa2Δ | for rendering strains dependent on | |
| | MFalpha1Δ MFalpha2Δ ste2Δ | peptide sensing. | |
| | ste3Δ sst2Δ far1Δ bar1Δ | | |
| | ste12::ste12* ste2::TDH3p- | | |
| | Ca.Ste2 sec4::OSR4p-Sec4 | | |
| ySB188 | MATa leu2Δ0 met15Δ0 ura3Δ0 | | This study |
| | his3Δ1 MFa1Δ MFa2Δ | | |
| | MFalpha1Δ MFalpha2Δ ste2Δ | | |
| | ste3Δ sst2Δ far1Δ bar1Δ | | |
| | ste12::ste12* ste2::TDH3p- | | |
| | Vp1.Ste2 sec4::OSR4p-Sec4 | | |
| yJB416 | MATa leu2Δ0 met15Δ0 ura3Δ0 | Parent GPCR integration strains | This study |
| | his3Δ1 MFa1Δ MFa2Δ | for constructing the 2-yeast linker | |
| | MFalpha1Δ MFalpha2Δ ste2Δ | strains, ring, bus -and tree | |
| | ste3Δ sst2Δ far1Δ bar1Δ | topologies; derived from yNA899. | |
| | ste2::TDH3p-Kp.Ste2 | | |
| yJB418 | MATa leu2Δ0 met15Δ0 ura3Δ0 | | This study |
| | his3Δ1 MFa1Δ MFa2Δ | | |
| | MFalpha1Δ MFalpha2Δ ste2Δ | | |
| | ste3Δ sst2Δ far1Δ bar1Δ | | |
| | ste2::TDH3p-Cl.Ste2 | | |
| yJB421 | MATa leu2Δ0 met15Δ0 ura3Δ0 | | This study |
| | his3Δ1 MFa1Δ MFa2Δ | | |
| | MFalpha1Δ MFalpha2Δ ste2Δ | | |
| | ste3Δ sst2Δ far1Δ bar1Δ | | |
| | ste2::TDH3p-Cgu.Ste2 | | |
| yJB422 | MATa leu2Δ0 met15Δ0 ura3Δ0 | | This study |
| | his3Δ1 MFa1Δ MFa2Δ | | |
| | MFalpha1Δ MFalpha2Δ ste2Δ | | |
| | ste3Δ sst2Δ far1Δ bar1Δ | | |
| | ste2::TDH3p-Bc.Ste2 | | |
| yJB423 | MATa leu2Δ0 met15Δ0 ura3Δ0 | | This study |
| | his3Δ1 MFa1Δ MFa2Δ | | |
| | MFalpha1Δ MFalpha2Δ ste2Δ | | |
| | ste3Δ sst2Δ far1Δ bar1Δ | | |
| | ste2::TDH3p-Ca.Ste2 | | |
| yJB523 | MATa leu2Δ0 met15Δ0 ura3Δ0 | | This study |
| | his3Δ1 MFa1Δ MFa2Δ | | |
| | MFalpha1Δ MFalpha2Δ ste2Δ | | |
| | ste3Δ sst2Δ far1Δ bar1Δ | | |
| | ste2::TDH3p-Hj.Ste2 | | |
| ySB315 | MATa leu2Δ0 met15Δ0 ura3Δ0 | Strain encoding two GPCRs for | This study |
| | his3Δ1 MFa1Δ MFa2Δ | the implementation of branches in | |
| | MFalpha1Δ MFalpha2Δ ste2Δ | the tree-topologies. Derived from | |
| | ste3Δ sst2Δ far1Δ bar1Δ | yJB418 | |
| | ste2::TDH3p-Cl.Ste2 | | |
| | ste3::TDH3p-Sj.Ste2 | | |
| ySB316 | MATa leu2Δ0 met15Δ0 ura3Δ0 | Strain encoding two GPCRs for | This study |
| | his3Δ1 MFa1Δ MFa2Δ | the implementation of branches in | |
| | MFalpha1Δ MFalpha2Δ ste2Δ | the tree-topologies. Derived from | |
| | ste3Δ sst2Δ far1Δ bar1Δ | yJB422 | |
| | ste2::TDH3p-Bc.Ste2 | | |
| | ste3::TDH3p-So.Ste2 | | |

Media. Synthetic dropout media (SD) supplemented with appropriate amino acids; fully supplemented medium containing all amino acids plus uracil and adenine is referred to as synthetic complete (SC)⁶⁰. Yeast strains were also cultured in YEPD medium^{61,62}. Escherichia coli was grown in Luria Broth (LB) media. To select for E. coli plasmids with drug-resistant genes, carbenicillin (Sigma-Aldrich) or kanamycin (Sigma-Aldrich) were used at final concentrations of 75-200 μg/ml and 50 μg/ml respectively. Agar was added to 2% for preparing solid yeast media.

TABLE 10
Primers used in this study.

| Primer | Primer Sequence 5′→3′ | Application |
|---|---|---|
| BAR1_delta_C | GATATTTATATGCTATAAAGAAATTGTACTCCAGATTTCccaTATATGACCCT | CRISPR |
| | TCTAGAC | deletion of |
| BAR1_delta_W | TCATACCAAAATAAAAAGAGTGTCTAGAAGGGTCATATAtggGAAATCTGGAG | BAR1 gene and |
| | TACAATT | verification |
| BAR1_FWD | GGCTGCACTCATTCCGGTAC | |
| BAR1_RVS | ACGGACGTTTAGGATGACGTATTG | |
| BAR1.3_C | GCTATTTCTAGCTCTAAAACatatttagtttcatgtacaaCTGCCAATCGCAG | |
| | CTCCCAG | |
| BAR1.3_W | CTGGGAGCTGCGATTGGCAGttgtacatgaaactaaatatGTTTTAGAGCTAG | |
| | AAATAGC | |
| BAR1.5_C | GCTATTTCTAGCTCTAAAACaaataagtttcaaacaaagaGATCATTTATCTT | |
| | TCACTGC | |
| BAR1.5_W | GCAGTGAAAGATAAATGATCtctttgtttgaaacttatttGTTTTAGAGCTAG | |
| | AAATAGC | |
| FAR1_delta_C | AGCAAAAGCCTCGAAATACGGGCCTCGATTCCCGAACTAccaTAATAGATTGC | CRISPR |
| | CTTCTTA | deletion of |
| FAR1_delta_W | CCACTGGAAAGCTTCGTGGGCGTAAGAAGGCAATCTATTAtggTAGTTCGGGA | FAR1 gene and |
| | ATCGAGG | verification |
| FAR1_FWD | GTTAGGCGGGCAAGAGAGAC | |
| FAR1_RVS | CGGAACAAATTAGCCACATCGACG | |
| FAR1.3_C | GCTATTTCTAGCTCTAAAACgggtctgatgaattctttgcCTGCCAATCGCAG | |
| | CTCCCAG | |
| FAR1.3_W | CTGGGAGCTGCGATTGGCAGgcaaagaattcatcagacccGTTTTAGAGCTAG | |
| | AAATAGC | |
| FAR1.5_C | GCTATTTCTAGCTCTAAAACcttggtggagtgtgtattttGATCATTTATCTT | |
| | TCACTGC | |
| FAR1.5_W | GCAGTGAAAGATAAATGATCaaaatacacactccaccaagGTTTTAGAGCTAG | |
| | AAATAGC | |
| MF_Bb_C | AAAAGGGGCCTGTctcaCTAccaacatggttgacctggtctcatacaccaAGC | Homology |
| | TTCAGCCTCTCTTTTAT | primers for |
| MF_Bb_W | ATAAAAGAGAGGCTGAAGCTtggtgtatgagaccaggtcaaccatgttggTAG | construction of |
| | tgagACAGGCCCCTTT | Peptide |
| MF_Bc_C | AAAAGGGGCCTGTCTCACTAacatggttgacctggtctaccacaccaAGCTTC | expression |
| | AGCCTCTCTTTTAT | vectors via |
| MF_Bc_W | ATAAAAGAGAGGCTGAAGCTtggtgtggtagaccaggtcaaccatgtTAGTGA | Gibson Assembly |
| | GACAGGCCCCTTTT | |
| MF_Ca_C | AAAAGGGGCCTGTCTCACTAacctggttcgaagtaaccgaagttggtcaatct | |
| | gaaaccAGCTTCAGCCTCTCTTTTAT | |
| MF_Ca_W | ATAAAAGAGAGGCTGAAGCTggtttcagattgaccaacttcggttacttcgaa | |
| | ccaggtTAGTGAGACAGGCCCCTTTT | |
| MF_Ct_C | AAAAGGGGCCTGTCTCACTAaccgataacgtcggtgtttctgaacttgatcca | |
| | cttccacttAGCTTCAGCCTCTCTTTTAT | |
| MF_Ct_W | ATAAAAGAGAGGCTGAAGCTaagtggaagtggatcaagttcagaaacaccgac | |
| | gttatcggtTAGTGAGACAGGCCCCTTTT | |
| MF_EAEA_Bb_C | AGGAAAAGGGGCCTGTcTCAccaacatggttgacctggtctcatacaccaTCT | |
| | TTTATCCAAAGATACCC | |
| MF_EAEA_Bb_W | GGGTATCTTTGGATAAAAGAtggtgtatgagaccaggtcaaccatgttggTGA | |
| | gACAGGCCCCTTTTCCT | |
| MF_EAEA_Ct_C | AGGAAAAGGGGCCTGTcTCAaccgataacgtcggtgtttctgaacttgatcca | |
| | cttccacttTCTTTTATCCAAAGATACCC | |
| MF_EAEA_Ct_W | GGGTATCTTTGGATAAAAGAaagtggaagtggatcaagttcagaaacaccgac | |
| | gttatcggtTGAgACAGGCCCCTTTTCCT | |
| MF_EAEA_Hj_C | AGGAAAAGGGGCCTGTcTCAccaacatggttcaccgattctgtaacaccaTCT | |
| | TTTATCCAAAGATACCC | |
| MF_EAEA_Hj_W | GGGTATCTTTGGATAAAAGAtggtgttacagaatcggtgaaccatgttggTGA | |
| | gACAGGCCCCTTTTCCT | |
| MF_EAEA_Kp_C | AGGAAAAGGGGCCTGTcTCAaccgaatggttggttctttcgttgtttctccat | |
| | ctgaaTCTTTTATCCAAAGATACCC | |
| MF_EAEA_Kp_W | GGGTATCTTTGGATAAAAGAttcagatggagaaacaacgaaaagaaccaacca | |
| | ttcggtTGAgACAGGCCCCTTTTCCT | |
| MF_EAEA_Le_C | AAAAGGGGCCTGTCTCACTaaactggagagaatctaccgtatctggtccacat | |
| | ccaAGCTTCAGCCTCTCTTTTAT | |
| MF_EAEA_Le_Cnew | AGGAAAAGGGGCCTGTcTCAaactggagagaatctaccgtatctggtccacat | |
| | ccaTCTTTTATCCAAAGATACCC | |
| MF_EAEA_Le_W | ATAAAAGAGAGGCTGAAGCTtggatgtggaccagatacggtagattctctcca | |
| | gtttAGTGAGACAGGCCCCTTTT | |
| MF_EAEA_Le_Wnew | GGGTATCTTTGGATAAAAGAtggatgtggaccagatacggtagattctctcca | |
| | gttTGAgACAGGCCCCTTTTCCT | |
| MF_EAEA_Pd_C | AGGAAAAGGGGCCTGTcTCAaccacatggttgacctggtctccaacagaaTCT | |
| | TTTATCCAAAGATACCC | |
| MF_EAEA_Pd_W | GGGTATCTTTGGATAAAAGAttctgttggagaccaggtcaaccatgtggtTGA | |
| | gACAGGCCCCTTTTCCT | |
| MF_EAEA_Zr_C | AAAAGGGGCCTGTCTCACTAgaacattggttgacctgggtccaattcgatgaa | |
| | gtgAGCTTCAGCCTCTCTTTTAT | |
| MF_EAEA_Zr_Cnew | AGGAAAAGGGGCCTGTcTCAgaacattggttgacctgggtccaattcgatgaa | |
| | gtgTCTTTTATCCAAAGATACCC | |
| MF_EAEA_Zr_W | ATAAAAGAGAGGCTGAAGCTcacttcatcgaattggacccaggtcaaccaatg | |
| | ttcTAGTGAGACAGGCCCCTTTT | |
| MF_EAEA_Zr_Wnew | GGGTATCTTTGGATAAAAGAcacttcatcgaattggacccaggtcaaccaatg | |
| | ttcTGAgACAGGCCCCTTTTCCT | |
| MF_Hi_C | AAAAGGGGCCTGTctcaCTAccaacatggttcaccgattctgtaacaccaAGC | |
| | TTCAGCCTCTCTTTTAT | |
| MF_Hi_W | ATAAAAGAGAGGCTGAAGCTtggtgttacagaatcggtgaaccatgttggTAG | |
| | tgagACAGGCCCCTTTT | |
| MF_Kp_C | AAAAGGGGCCTGTctcaCTAaccgaatggttggttctttcgttgttctccatc | |
| | tgaaAGCTTCAGCCTCTCTTTTAT | |
| MF_Kp_W | ATAAAAGAGAGGCTGAAGCTttcagatggagaaacaacgaaaagaaccaacca | |
| | ttcggtTAGtgagACAGGCCCCTTTT | |
| MF_Le_C | AAAAGGGGCCTGTCTCACTAaactggagagaatctaccgtatctggtccacat | |
| | ccaAGCTTCAGCCTCTCTTTTAT | |
| MF_Le_W | ATAAAAGAGAGGCTGAAGCTtggatgtggaccagatacggtagattctctcca | |
| | gttTAGTGAGACAGGCCCCTTTT | |
| MF_Pb_C | AAAAGGGGCCTGTCTCACTAacaaccttgacctggtctggtacaccaAGCTTC | |
| | AGCCTCTCTTTTAT | |
| MF_Pb_W | ATAAAAGAGAGGCTGAAGCTtggtgtaccagaccaggtcaaggttgtTAGTGA | |
| | GACAGGCCCCTTTT | |
| MF_Pd_C | AAAAGGGGCCTGTctcaCTAaccacatggttgacctggtctccaacagaaAGC | |
| | TTCAGCCTCTCTTTTAT | |
| MF_Pd_W | ATAAAAGAGAGGCTGAAGCTttctgttggagaccaggtcaaccatgtggtTAG | |
| | tgagACAGGCCCCTTTT | |
| MF_Sc_C | AAAAGGGGCCTGTCTCACTAgtacattggttgacctggcttcaattgcaacca | |
| | gtgccaAGCTTCAGCCTCTCTTTTAT | |
| MF_Sc_W | ATAAAAGAGAGGCTGAAGCTtggcactggttgcaattgaagccaggtcaacca | |
| | atgtacTAGTGAGACAGGCCCCTTTT | |
| MF_Vp_C | AAAAGGGGCCTGTCTCACTAgtagattggttgaccgttgtccaattccaacca | |
| | gtgccaAGCTTCAGCCTCTCTTTTAT | |
| MF_Vp_W | ATAAAAGAGGCTGAAGCTtggcactggttggaattggacaacggtcaaccaat | |
| | ctacTAGTGAGACAGGCCCCTTTT | |
| MF_Zr_C | AAAAGGGGCCTGTCTCACTAgaacattggttgacctgggtccaattcgatgaa | |
| | gtgAGCTTCAGCCTCTCTTTTAT | |
| MF_Zr_W | ATAAAAGAGAGGCTGAAGCTcacttcatcgaattggacccaggtcaaccaatg | |
| | ttcTAGTGAGACAGGCCCCTTTT | |
| MF-EAEA_Bc_C | AGGAAAAGGGGCCTGTCTCAacatggttgacctggtctaccacaccaTCTTTT | |
| | ATCCAAAGATACCC | |
| MF-EAEA_Bc_W | GGGTATCTTTGGATAAAAGAtggtgtggtagaccaggtcaaccatgtTGAGAC | |
| | AGGCCCCTTTTCCT | |
| MF-EAEA_Ca_C | AGGAAAAGGGGCCTGTCTCAacctggttcgaagtaaccgaagttggtcaatct | |
| | gaaaccTCTTTTATCCAAAGATACCC | |
| MF-EAEA_Ca_W | GGGTATCTTTGGATAAAAGAggtttcagattgaccaacttcggttacttcgaa | |
| | ccaggtTGAGACAGGCCCCTTTTCCT | |
| MF-EAEA_Pb_C | AGGAAAAGGGGCCTGTCTCAacaaccttgacctggtctggtacaccaTCTTTT | |
| | ATCCAAAGATACCC | |
| MF-EAEA_Pb_W | GGGTATCTTTGGATAAAAGAtggtgtaccagaccaggtcaaggttgtTGAGAC | |
| | AGGCCCCTTTTCCT | |
| MF-EAEA_Sc_C | AGGAAAAGGGGCCTGTCTCAgtacattggttgacctggcttcaattgcaacca | |
| | gtgccaTCTTTTATCCAAAGATACCC | |
| MF-EAEA_Sc_W | GGGTATCTTTGGATAAAAGAtggcactggttgcaattgaagccaggtcaacca | |
| | atgtacTGAGACAGGCCCCTTTTCCT | |
| MF-EAEA_Vp_C | AGGAAAAGGGGCCTGTCTCAgtagattggttgaccgttgtccaattccaacca | |
| | gtgccaTCTTTTATCCAAAGATACCC | |
| MF-EAEA_Vp_W | GGGTATCTTTGGATAAAAGAtggcactggttggaattggacaacggtcaacca | |
| | atctacTGAGACAGGCCCCTTTTCCT | |
| MFa.5_C | GCTATTTCTAGCTCTAAAACgaagacacctttgataatatGATCATTTATCTT | CRISPR |
| | TCACTGC | deletion of |
| MFa.5_W | GCAGTGAAAGATAAATGATCatattatcaaaggtgtcttcGTTTTAGAGCTAG | MFA1 gene and |
| | AAATAGC | verification |
| MFa1_FWD | CTGCTACGGTTGGCCCATAC | |
| MFa1_RVS | ACTTCACGGTAGGTGGTAAGC | |
| MFa1.5_C | GCTATTTCTAGCTCTAAAACtcttttcactgctggtctttGATCATTTATCTT | |
| | TCACTGC | |
| MFa1.5_W | GCAGTGAAAGATAAATGATCaaagaccagcagtgaaaagaGTTTTAGAGCTAG | |
| | AAATAGC | |
| MFa1delta_C | AAGATAAAGGAGGGAGAACAACGTTTTTGTACGCAGAAATTCTATTCGATGGC | |
| | TTTGTACTTATTTTGGTTTTATCCG | |
| MFa1delta_W | TCGGATAAAACCAAAATAAGTACAAAGCCATCGAATAGAATTTCTGCGTACAA | |
| | AAACGTTGTTCTCCCTCCTTTATCT | |
| MFa2_FWD | TTCCATCCACTTCTTCTGTCGTTC | CRISPR |
| MFa2_RVS | GGGTGGTTCATCTTTCATTTCCTGC | deletion of |
| MFa2.3_C | GCTATTTCTAGCTCTAAAACtctgagtggcttgtgtggaaCTGCCAATCGCAG | MFA2 gene and |
| | CTCCCAG | verification |
| MFa2.3_W | CTGGGAGCTGCGATTGGCAGttccacacaagccactcagaGTTTTAGAGCTAG | |
| | AAATAGC | |
| MFa2.5_C | GCTATTTCTAGCTCTAAAACtctgagtggcttgtgtggaaGATCATTTATCTT | |
| | TCACTGC | |
| MFa2.5_W | GCAGTGAAAGATAAATGATCttccacacaagccactcagaGTTTTAGAGCTAG | |
| | AAATAGC | |
| MFa2delta_C | AGGGTAGATATTGATTTGACCTCTTGGTTGTCGTCAAAAATAAGGTTGGTAGT | |
| | TATTGTTGTATGAAGATGATAGCTCG | |
| MFa2delta_W | GCGAGCTATCATCTTCATACAACAATAACTACCAACCTTATTTTGACGACAAC | |
| | CAAGAGGTCAAATCAATATCTACC | |
| MFalpha1_FWD | TGCGCTAAATAGACATCCCGTTC | CRISPR |
| MFalpha1_RVS | CAGAGGCATCATAATCAGGGAGTG | deletion of |
| MFalpha1.3_C | gctatttctagctctaaaacggttttaactgcaaccaatgCTGCCAATCGCAG | MFalpha1 |
| | CTCCCAG | gene and |
| MFalpha1.3_W | CTGGGAGCTGCGATTGGCAGcattggttgcagttaaaaccgttttagagctag | verification |
| | aaatagc | |
| MFalpha1.5_C | GCTATTTCTAGCTCTAAAACTCAATTTTTACTGCAGTTTTGATCATTTATCTT | |
| | TCACTGC | |
| MFalpha1.5_W | GCAGTGAAAGATAAATGATCAAAACTGCAGTAAAAATTGAGTTTTAGAGCTAG | |
| | AAATAGC | |
| MFalpha1delta_C | GTCGACTTTGTTACATCTACACTGTTGTTATCAGTCGGGCTCTTTTAATCGTT | |
| | TATATTGTGTATGAAATTGATAGTTT | |
| MFalpha1delta_W | CAAACTATCAATTTCATACACAATATAAACGATTAAAAGAGCCCGACTGATAA | |
| | CAACAGTGTAGATGTAACAAAGTCGA | |
| MFalpha2_FWD | GGCGACGCCTGTAGTGATTG | CRISPR |
| MFalpha2_RVS | GGGAACCTTGCTTGCAGACAG | deletion of |
| MFalpha2.3_C | gctatttctagctctaaaacGGCTTGAGTTGCAACCAGTGCTGCCAATCGCAG | MFalpha2 |
| | CTCCCAG | gene and |
| MFalpha2.3_W | CTGGGAGCTGCGATTGGCAGCACTGGTTGCAACTCAAGCCgttttagagctag | verification |
| | aaatagc | |
| MFalpha2.5_C | GCTATTTCTAGCTCTAAAACttctcacttttatttagcgGATCATTTATCTTT | |
| | CACTGC | |
| MFalpha2.5_W | GCAGTGAAAGATAAATGATCcgctaaaataaaagtgagaaGTTTTAGAGCTAG | |
| | AAATAGC | |
| MFalpha2delta_C | AAGAAATCGAGAGGGTTTAGAAGTAGTTTAGGGTCATTTTTTTCTCCAATATG | |
| | TGAATTTACTGGAATTTGATGCAGGT | |
| MFalpha2delta_W | CACCTGCATCAAATTCCAGTAAATTCACATATTGGAGAAAAAAATGACCCTAA | |
| | ACTACTTCTAAACCCTCTCGATTTCT | |
| SST2_donor_C | GTGCAATTGTACCTGAAGATGAGTAAGACTCTCAATGAAAccaCTTACAAC | CRISPR |
| SST2_donor_W | GTTATAGGTTCAATTTGGTAATTAAAGATAGAGTTGTAAGtggTTTCATTGA | deletion of |
| SST2_FWD | TGACTAGGACTTGGATTTGGTTGC | SST2 gene and |
| SST2_RVS | GCGCTCACGTTAGTCACATCTC | verification |
| sst2.3_C | GCTATTTCTAGCTCTAAAACgtcagacgtatacaaagatgCTGCCAATCGCAG | |
| | CTCCCAG | |
| sst2.3_W | CTGGGAGCTGCGATTGGCAGcatctttgtatacgtctgacGTTTTAGAGCTAG | |
| | AAATAGC | |
| sst2.5_C | GCTATTTCTAGCTCTAAAACatttttatccaccatcttacGATCATTTATCTT | |
| | TCACTGC | |
| sst2.5_W | GCAGTGAAAGATAAATGATCgtaagatggtggataaaaatGTTTTAGAGCTAG | |
| | AAATAGC | |
| STE12_FWD | ACTCTTCGCGGTCAGGTCTC | CRISPR |
| STE12_RVS | GGCAATACTACGTTGGTATCAAAATAGTGG | deletion of |
| STE12.3_C | gctatttctagctctaaaactcgattggtatctacctcaaCTGCCAATCGCAG | STE12 gene and |
| | CTCCCAG | verification |
| STE12.3_W | CTGGGAGCTGCGATTGGCAGttgaggtagataccaatcgagttttagagctag | |
| | aaatagc | |
| STE12.5_C | GCTATTTCTAGCTCTAAAACctgttctactattggttattGATCATTTATCTT | |
| | TCACTGC | |
| STE12.5_W | GCAGTGAAAGATAAATGATCaataaccaatagtagaacagGTTTTAGAGCTAG | |
| | AAATAGC | |
| STE12delta_C | TTTTTAATTCTTGTATCATAAATTCAAAAATTATATTATACCTTGGTGAACAA | |
| | GACAATTCAAATAAAGAAAGCGGTTC | |
| STE12delta_W | GGAACCGCTTTCTTTATTTGAATTGTCTTGTTCACCAAGGTATAATATAATTT | |
| | TTGAATTTATGATACAAGAATTAAAA | |
| STE2_FWD | TAGGACCTGTGCCTGGCAAG | CRISPR |
| STE2_RVS | CATCACAATATACTAGCAGTGGCACC | deletion of |
| STE2.3_C | gctatttctagctctaaaacgaactttctggcttcctcatCTGCCAATCGCAG | STE2 gene and |
| | CTCCCAG | verification |
| STE2.3_W | CTGGGAGCTGCGATTGGCAGatgaggaagccagaaagttcgttttagagctag | |
| | aaatagc | |
| STE2.5_C | GCTATTTCTAGCTCTAAAACcatcagaCATttttgattctGATCATTTATCTT | |
| | TCACTGC | |
| STE2.5_W | GCAGTGAAAGATAAATGATCagaatcaaaaATGtctgatgGTTTTAGAGCTAG | |
| | AAATAGC | |
| STE2delta_C | GAAGGTCACGAAATTACTTTTTCAAAGCCGTAAATTTTGATTTTGATTCTTGG | |
| | ATATGGTTCTTAACGGTGCATTTTTA | |
| STE2delta_W | TTAAAAATGCACCGTTAAGAACCATATCCAAGAATCAAAATCAAAATTTACGG | |
| | CTTTGAAAAAGTAATTTCGTGACCTT | |
| STE3_FWD | TGCGTTTCATTTGGCCGTTATCAC | CRISPR |
| STE3_RVS | CTTGGTGTGCAGAATAGTGATAGAGC | deletion of |
| STE3.3_C | gctatttctagctctaaaacGCAGTATTTTCTGAACTATGCTGCCAATCGCAG | STE3 gene and |
| | CTCCCAG | verification |
| STE3.3_W | CTGGGAGCTGCGATTGGCAGCATAGTTCAGAAAATACTGCgttttagagctag | |
| | aaatagc | |
| STE3.5_C | GCTATTTCTAGCTCTAAAACTATTATTGCTGACTTGTATGGATCATTTATCTT | |
| | TCACTGC | |
| STE3.5_W | GCAGTGAAAGATAAATGATCCATACAAGTCAGCAATAATAGTTTTAGAGCTAG | |
| | AAATAGC | |
| STE3delta_C | AATACTCCTAGTCCAGTAAATATAATGCGACACTCTTGTGGAAAATTTTGATA | |
| | GTATTTTGCCTTTCCTACACAAATTT | |
| STE3delta_W | TAAATTTGTGTAGGAAAGGCAAAATACTATCAAAATTTTCCACAAGAGTGTCG | |
| | CATTATATTTACTGGACTAGGAGTAT | |
| ScSte2_FWD | ACCAAGAACTTAGTTTCGACGGATACTAGTAAAatgtctgatgcggctccttc | Homology |
| ScSte2_RVS | ACGAAATTACTTTTTCAAAGCCGTCTCGAGCTAtaaattattattatcttcag | primers for |
| | tccagaa | construction of |
| CaSte2_FWD | gtgtcgTCTAGAAAAatgaatatcaattcaactttcatacc | GPCR expression |
| CaSte2_RVS | gcaagtCTCGAGCtacactcttttgatggtgatttg | vectors via |
| AgSte2_FWD | ACCAAGAACTTAGTTTCGACGGATACTAGTAAAatgggtgaagaggtatctag | Gibson Assembly |
| | c | |
| AgSte2_RVS | ACGAAATTACTTTTTCAAAGCCGTCTCGAGctagttgcaatcacttccggt | |
| BcSte2_FWD | ACCAAGAACTTAGTTTCGACGGATACTAGTAAAatggcttctaactcttctaa | |
| | cttc | |
| BcSte2_RVS | ACGAAATTACTTTTTCAAAGCCGTCTCGAGctaagccttttgaacaccgtaag | |
| CgSte2_FWD | ACCAAGAACTTAGTTTCGACGGATACTAGTAAAatggagatgggctacgatcc | |
| CgSte2_RVS | ACGAAATTACTTTTTCAAAGCCGTCTCGAGctatttgtcacactgactttgtt | |
| | g | |
| FgSte2_FWD | ACCAAGAACTTAGTTTCGACGGATACTAGTAAAatgtctaaggaagttttcga | |
| | ccca | |
| FgSte2_RVS | ACGAAATTACTTTTTCAAAGCCGTCTCGAGctacaatggagctctgattcttt | |
| | c | |
| KlSte2_FWD | ACCAAGAACTTAGTTTCGACGGATACTAGTAAAatgtcagaagagatacccag | |
| | tttg | |
| KlSte2_RVS | ACGAAATTACTTTTTCAAAGCCGTCTCGAGctatcttaattctttgaatacgg | |
| | ttttc | |
| LeSte2_FWD | ACCAAGAACTTAGTTTCGACGGATACTAGTAAAatggacgaagcaatcaatgc | |
| | aaac | |
| LeSte2_RVS | ACGAAATTACTTTTTCAAAGCCGTCTCGAGctattttttcaacatagtcactt | |
| | c | |
| MoSte2_FWD | ACCAAGAACTTAGTTTCGACGGATACTAGTAAAatggaccaaactttgtctgc | |
| | tac | |
| MoSte2_RVS | ACGAAATTACTTTTTCAAAGCCGTCTCGAGctacaatctttcttctcttcttt | |
| | cga | |
| PbSte2_FWD | ACCAAGAACTTAGTTTCGACGGATACTAGTAAAatggcaccctcattcgacc | |
| PbSte2_RVS | ACGAAATTACTTTTTCAAAGCCGTCTCGAGctaggcctttgtgccagcttc | |
| SpSte2_FWD | ACCAAGAACTTAGTTTCGACGGATACTAGTAAAatgagacaaccatggtggaa | |
| | ag | |
| SpSte2_RVS | ACGAAATTACTTTTTCAAAGCCGTCTCGAGctacgtccactttttagtttcag | |
| | attc | |
| Vp1Ste2_FWD | ACCAAGAACTTAGTTTCGACGGATACTAGTAAAatgagttcccaatcacaccc | |
| | a | |
| Vp1Ste2_RVS | ACGAAATTACTTTTTCAAAGCCGTCTCGAGctatgaagtccttgtgatatcgt | |
| | tac | |
| Vp2Ste2_FWD | ACCAAGAACTTAGTTTCGACGGATACTAGTAAAatgtcaggaattgatgatat | |
| | gggt | |
| Vp2Ste2_RVS | ACGAAATTACTTTTTCAAAGCCGTCTCGAGctattgttttctaaatgttattc | |
| | tttttg | |
| ZbSte2_FWD | ACCAAGAACTTAGTTTCGACGGATACTAGTAAAatgtctggttggctaacaac | |
| | ac | |
| ZbSte2_RVS | ACGAAATTACTTTTTCAAAGCCGTCTCGAGctaccatttgacgttcttcttca | |
| | aa | |
| ZrSte2_FWD | ACCAAGAACTTAGTTTCGACGGATACTAGTAAAatgagtgagattaacaattc | |
| | tacctac | |
| ZrSte2_RVS | ACGAAATTACTTTTTCAAAGCCGTCTCGAGctataatttctttaggataattt | |
| | ttttact | |
| SsSte2_FWD | ACACCAAGAACTTAGTTTCGACGGATACTAGTAAAatggatactagtatcaat | |
| | actctcaaccct | |
| SsSte2_RVS | ACGAAATTACTTTTTCAAAGCCGTCTCGAGCTAgctttcagaaaagtgagagg | |
| | tcgtt | |
| SjSte2_FWD | ACCAAGAACTTAGTTTCGACGGATACTAGTAAAatgtactcctgggacgaatt | |
| | c | |
| SjSte2_RVS | ACGAAATTACTTTTTCAAAGCCGTCTCGAGCTAtggcaaagtttcttcggtct | |
| | t | |
| ScaSte2_FWD | ACCAAGAACTTAGTTTCGACGGATACTAGTAAAatgtctgacgctccaccac | |
| ScaSte2_RVS | ACGAAATTACTTTTTCAAAGCCGTCTCGAGCTAttgcttctgacggtgatctt | |
| PrSte2_FWD | ACCAAGAACTTAGTTTCGACGGATACTAGTAAAatggcttctatggttccacc | |
| | a | |
| PrSte2_RVS | ACGAAATTACTTTTTCAAAGCCGTCTCGAGCTAgacgatggagttgttacgtt | |
| | g | |
| MgSte2_FWD | ACCAAGAACTTAGTTTCGACGGATACTAGTAAAatggtggtaacagctccacc | |
| | t | |
| MgSte2_RVS | ACGAAATTACTTTTTCAAAGCCGTCTCGAGCTAgtcggaacggactgagtatg | |
| CguSte2_FWD | ACCAAGAACTTAGTTTCGACGGATACTAGTAAAatgaagtcctgctccatcgg | |
| CguSte2_RVS | ACGAAATTACTTTTTCAAAGCCGTCTCGAGCTAgatggaggtggagtcgatca | |
| CtSte2_FWD | ACCAAGAACTTAGTTTCGACGGATACTAGTAAAatggacatcaacaacaccat | |
| | c | |
| CtSte2_RVS | ACGAAATTACTTTTTCAAAGCCGTCTCGAGCTAgaccttcttgtaggtgactt | |
| CpSte2_FWD | ACCAAGAACTTAGTTTCGACGGATACTAGTAAAatgaacaagattgtctccaa | |
| | gtt | |
| CpSte2_RVS | ACGAAATTACTTTTTCAAAGCCGTCTCGAGCTAttggttgttgtgagcggtct | |
| SoSte2_FWD | ACCAAGAACTTAGTTTCGACGGATACTAGTAAAatgcgtgaaccatggtggaa | |
| | g | |
| SoSte2_RVS | ACGAAATTACTTTTTCAAAGCCGTCTCGAGCTAtggccacttcttgatttcgg | |
| | t | |
| SnSte2_FWD | ACCAAGAACTTAGTTTCGACGGATACTAGTAAAatggcttctatggttccacc | |
| | a | |
| SnSte2_RVS | ACGAAATTACTTTTTCAAAGCCGTCTCGAGCTAacctctttcaccgacttcac | |
| CcSte2_FWD | ACCAAGAACTTAGTTTCGACGGATACTAGTAAAatggctgctagaattatccc | |
| | a | |
| CcSte2_RVS | ACGAAATTACTTTTTCAAAGCCGTCTCGAGCTAgaccatgttttcagaaccaa | |
| | c | |
| GcSte2_FWD | ACCAAGAACTTAGTTTCGACGGATACTAGTAAAatggccgaagactccatctt | |
| | c | |
| GcSte2_RVS | ACGAAATTACTTTTTCAAAGCCGTCTCGAGCTActtacgggtgacgtcggtt | |
| SkSte2_FWD | ACCAAGAACTTAGTTTCGACGGATACTAGTAAAatgtccggtaagcaagact | |
| | tg | |
| SkSte2_RVS | ACGAAATTACTTTTTCAAAGCCGTCTCGAGCTAggtggtcatcaagatcttgg | |
| | a | |
| AnSte2_FWD | ACCAAGAACTTAGTTTCGACGGATACTAGTAAAatggctacccacaaccaaat | |
| | c | |
| AnSte2_RVS | ACGAAATTACTTTTTCAAAGCCGTCTCGAGCTAgacgtcaaaagattcacgac | |
| | g | |
| AoSte2_FWD | ACCAAGAACTTAGTTTCGACGGATACTAGTAAAatggactctaagttcgaccc | |
| | a | |
| AoSte2_RVS | ACGAAATTACTTTTTCAAAGCCGTCTCGAGCTAcaatctttgacaggagtgga | |
| | c | |
| BbSte2_FWD | ACCAAGAACTTAGTTTCGACGGATACTAGTAAAatggatggttcttctgctcc | |
| | a | |
| BbSte2_RVS | ACGAAATTACTTTTTCAAAGCCGTCTCGAGCTAggcgaagttatcacgttgca | |
| | t | |
| ClSte2_FWD | ACCAAGAACTTAGTTTCGACGGATACTAGTAAAatgaacccagctgacatcaa | |
| | c | |
| ClSte2_RVS | ACGAAATTACTTTTTCAAAGCCGTCTCGAGAGCTAtcaatctatgggtggtga | |
| | c | |
| CnSte2_FWD | ACCAAGAACTTAGTTTCGACGGATACTAGTAAAatggactcctacttgttgaa | |
| | cc | |
| CnSte2_RVS | ACGAAATTACTTTTTCAAAGCCGTCTCGAGCTActtcataccgatgtcggtgt | |
| | t | |
| AfSte2_FWD | ACCAAGAACTTAGTTTCGACGGATACTAGTAAAatgaactccaccttcgaccc | |
| | a | |
| AfSte2_RVS | ACGAAATTACTTTTTCAAAGCCGTCTCGAGCTAaatatcaccgtgggcgtcct | |
| | t | |
| PdSte2_FWD | ACCAAGAACTTAGTTTCGACGGATACTAGTAAAatgtccactgccaacgttca | |
| | t | |
| PdSte2_RVS | ACGAAATTACTTTTTCAAAGCCGTCTCGAGCTAgaagatgtcctctctctcga | |
| | t | |
| HjSte2_FWD | ACCAAGAACTTAGTTTCGACGGATACTAGTAAAatgtcttccttcgacccata | |
| | c | |
| HjSte2_RVS | ACGAAATTACTTTTTCAAAGCCGTCTCGAGCTAagaggaagaagtgttggcga | |
| | t | |
| TmSte2_FWD | ACCAAGAACTTAGTTTCGACGGATACTAGTAAAatggagcaaatcccagtcta | |
| | c | |
| TmSte2_RVS | ACGAAATTACTTTTTCAAAGCCGTCTCGAGCTAggcgaattcgaaacctcttt | |
| | c | |
| DbSte2_FWD | ACCAAGAACTTAGTTTCGACGGATACTAGTAAAatggaccacaacacccaaca | |
| | c | |
| DbSte2_RVS | ACGAAATTACTTTTTCAAAGCCGTCTCGAGCTAgtcatcgtggtcaccaacgt | |
| SheSte2_FWD | ACCAAGAACTTAGTTTCGACGGATACTAGTAAAatgaaacccgccgctggac | |
| SheSte2_RVS | ACGAAATTACTTTTTCAAAGCCGTCTCGAGCTAgaccatgtcccttctgacct | |
| YlSte2_FWD | ACCAAGAACTTAGTTTCGACGGATACTAGTAAAatgcaattgccaccacgtcc | |
| | a | |
| YlSte2_RVS | ACGAAATTACTTTTTCAAAGCCGTCTCGAGCTAcatcttttcgtcacattcga | |
| | aac | |
| TdSte2_FWD | ACCAAGAACTTAGTTTCGACGGATACTAGTAAAatgtctgactccgcccaaaa | |
| | c | |
| TdSte2_RVS | ACGAAATTACTTTTTCAAAGCCGTCTCGAGCTAccatttcaaggaggccttac | |
| | g | |
| KpSte2_FWD | ACCAAGAACTTAGTTTCGACGGATACTAGTAAAatggaagaatactccgactc | |
| | c | |
| KpSte2_RVS | ACGAAATTACTTTTTCAAAGCCGTCTCGAGCTAgaagtgcaaatcttcggagg | |
| | t | |
| CauSte2_FWD | ACCAAGAACTTAGTTTCGACGGATACTAGTAAAatggaattcactggtgacat | |
| CauSte2_RVS | ACGAAATTACTTTTTCAAAGCCGTCTCGAGCTActaaacagttctgttgttca | |
| | agtt | |
| NcSte2_FWD | ACCAAGAACTTAGTTTCGACGGATACTAGTAAAatggcgtcctcttcctcac | |
| NcSte2_RVS | ACGAAATTACTTTTTCAAAGCCGTCTCGAGCTActcgaatgatctaggcttcg | |
| | t | |
| BmSte2_FWD | ACCAAGAACTTAGTTTCGACGGATACTAGTAAAatggcctcaaacggctg | |
| BmSte2_RVS | ACGAAATTACTTTTTCAAAGCCGTCTCGAGCTAgtcgtcaccgattagtgtat | |
| | cta | |
| Ste2_Int_Hom_FWD | GAATTTAAGCAGGCCAACGTCCATACTGCTTAGGACCTGTGCCTGGCAAGTCG | Integration of |
| | CAGATTGAAGagtttatcattatcaatactgc | CPCRs into |
| Ste2_Int_Hom_RVS | CTCGTAAAAGCAAAGGTGG | Ste2Δ locus |
| Ste2_Int_ColPCR_FWD | GTCTCGTGCATTAAGACAGGC | Verification |
| Ste2_Int_ColPCR_RVS | CCTGAGAGTTCTAGATCATGGCAAG | of GPCR |
| | | integration |
| | | into Ste2Δ |
| | | locus |
| CaSte2_ColPCR_FWD | TCCAGGATTAGATCAACCAATTC | Determination |
| CaSte2_ColPCR_RVS | GATTTGAAAGGCAACAACAATC | of strain |
| KpSte2_ColPCR_FWD | GGACGACTACCACTTCTACGTC | ratios in |
| KpSte2_ColPCR_RVS | AGTATCTGTTCTTCCAGGCGA | mixed culture |
| BcSte2_ColPCR_FWD | CTTGATGGCTGACGGTATCA | |
| BcSte2_ColPCR_RVS | CTCTTGATGTCGTCCAAGTTCTTAC | |
| Ste3_Int_Hom_FWD | GGTATGGGTGCTAATTTTCGTTAGAAGCGCTGGTACAATTTTCTCTGTCATTG | Integration of |
| | TGACACTA AGTTTATCATTATCAATACTGC | GPCRs into |
| Ste3_Int_Hom_RVS | GTAAAAATAAAATACTCCTAGTCCAGTAAATATAATGCGACACTCTTGTGGAA | Ste3Δ locus |
| | ATTACTTTTTCAAAGCCG | |
| Ste3_Int_ColPCR_FWD | CCTATATTATTGTACCACATTGC | Verification |
| Ste3_Int_ColPCR_RVS | CTGATGAGCTCATCGTTAC | of GPCR |
| | | integration |
| | | into Ste3Δ |
| | | locus |
| Ste12⁺_Int_FWD | CGAAGAAAACACACTTTTATAGCGGAACCGCTTTCTTTATTTGAATTGTCTTG | Replacing the |
| | TTCACCAAGGATGGATACTAGTGACTACAAGGACCAC | DNA binding |
| Ste12⁺_Int_RVS | CTTCTTCGTCTCTGCCC | domain of |
| | | Ste12 by ZF43-8 |
| | | (Ste12⁺) |
| Ste12⁺_Int_ColPCR_FWD | CGGAGAGCTCGTTTCAAAATG | Verification |
| Ste12⁺_Int_ColPCR_RVS | CTTCTTCGTCTCTGCCC | of Ste12⁺ |
| CYC1t_Int_Hom-FWD | GTAGACATACTGTATATACACGAGGGCGTATCGTTCACCAGAAAGAATATAAA | Replacing Sec4 |
| | CATAACAAGATAAACATGTAATTAGTTATGTCAC | promoter with |
| CYC1t_Int_Hom_RVS | GAGTCCTCACTCTATTAATATTTTCGAGTCCTCACTCTGTCGACCTCGAGGGG | CYC1t-OSR2 |
| | GGGCCCGGTACCCAATTCGCCGGCCGCAAATTAAAGC | |
| OSR2_Int_Hom_RWD | GTACGCATGTAACATTATACTGAAAACCTTGCTTGAGAAGGTTTTGGGACGCT | |
| | CGAAGGCTTTAATTTGCGGCCGGCGAATTGGGTACC | |
| OSR2_Int_Hom_RVS | GAGTCATAGCTCTTTCCATTACCTGAGGACGCTGAGACAGTTCTCAAGCCTGA | |
| | CATTTTTTATCTAGATTAGTGTGTGTATTTGTGTTTG | |
| OSR4_Int_Hom_FWD | CGAGAGATTTGCAAAGGGTCTCGACGTCAACAAATACACGTCGAAAGAAAGAC | Replacing Sec4 |
| | AAAAGTTATCCAAAACGGATggcgaattgggtac | promoter with |
| OSR4_Int_Hom_RVS | ATAAAATTTTCATAATAGAGTCATAGCTCTTTCCATTACCGGATGAAGCAGAA | OSR4 |
| | ACAGTTCTCAAGCCTGACATCTAGATTTTTTCGATGC | |
| Sec4_Int_ColPCR_FWD | GGAATTTGTTGTCAGC | Verification of |
| Sec4_Int_ColPCR_RVS | GATACCCATAGCACCAC | Sec4 promoter |
| | | replacement |
| | | with OSRs |

Materials. Synthetic peptides (≥95% purity) were obtained from GenScript (Piscataway, N.J., USA). S. cerevisiae alpha-factor was obtained from Zymo Research (Irvine, Calif., USA). Polymerases, restriction enzymes and Gibson assembly mix were obtained from New England Biolabs (NEB) (Ipswich, Mass., USA). Media components were obtained from BD Bioscience (Franklin Lakes, N.J., USA) and Sigma Aldrich (St. Luis, Mo., USA). Primers and synthetic DNA (gBlocks) were obtained from Integrated DNA Technologies (IDT, Coralville, Iowa, USA). Primers used in this study are listed in Table 10. Plasmids were cloned and amplified in E. coli C3040 (NEB). Sterile, black, clear-bottom 96-well microtiter plates were obtained from Corning (Corning Inc.).

Bioinformatic extraction of GPCR genes and peptide precursors. A database of fungal receptors was curated from the InterPro (IPR000366)⁶³ and PFAM (PF02116) families⁶⁴. Sequence identifiers were standardized using the UniProt ID mapping tool (http://www.uniprot.org/uploadlists/). UniProt IDs were used to programmatically retrieve associated taxonomic information. Taxonomic information was used to filter out non-fungal sequences and fragments. The amino acid sequences of the corresponding peptide ligands were derived in a similar approach. Sequences were validated by multiple sequence alignment using Clustal Omega⁶⁵. The amino acid sequences, as well as the % identity for all Ste2-like GPCRs and peptide precursors are listed in Table 3, 4 and 9.

TABLE 3
Summary of GPCRs and peptide ligands. Ascomycete species used for genomic GPCR
extraction, inferred peptide ligands (Table 4 lists peptide precursors used
for inference of peptide ligands) and % identity of a given GPCR's amino acid
sequence or a given motif stretch when compared to the S. cerevisiae Ste2
(see also FIG. 2). GPCRs are organized by % identity (full Ste2). For species codes
labeled with a reference, the #1 peptide candidate has been postulated or tested
before. References indicated by superscript numbers in Table 3 and Table 4 are as
follows: 1 = Kurjan, J. & Herskowitz, I. Structure of a Yeast Pheromone Gene
(Mf-Alpha)-a Putative Alpha-Factor Precursor Contains 4 Tandem Copies of Mature
Alpha-Factor. Cell 30, 933-943 (1982); 2 = Martin, S. H., Wingfield, B. D., Wingfield,
M. J. & Steenkamp, E. T. Causes and Consequences of Variability in Peptide Mating
Pheromones of Ascomycete Fungi. Mol Biol Evol 28, 1987-2003 (2011); 3 = Egelmitani,
M. & Hansen, M. T. Nucleotide-Sequence of the Gene Encoding the Saccharomyces-Kluyveri
Alpha-Mating Pheromone. Nucleic Acids Res 15, 6303-6303 (1987); 4 = Wong, S.,
Fares, M. A., Zimmermann, W., Butler, G. & Wolfe, K. H. Evidence from comparative
genomics for a complete sexual cycle in the ‘asexual’ pathogenic yeast Candida glabrata.
Genome Biol 4 (2003); 5 = Bennett, R. J., Uhl, M. A., Miller, M. G. & Johnson, A. D.
Identification and characterization of a Candida albicans mating pheromone. Molecular
and cellular biology 23, 8189-8201 (2003); 6 = Imai, Y. & Yamamoto, M. The Fission Yeast
Mating Pheromone P-Factor Its Molecular-Structure, Gene Structure, and Ability to Induce
Gene-Expression and G(1) Arrest in the Mating Partner. Gene Dev 8, 328-338 (1994);
7 = Gomes-Rezende, J. A. et al. Functionality of the Paracoccidioides mating
alpha-pheromone-receptor system. PloS one 7, e47033 (2012); 8 = Dyer, P. S., Paoletti, M.
& Archer, D. B. Genomics reveals sexual secrets of Aspergillus. Microbiology 149,
2301-2303 (2003); 9 = Bobrowicz, P., Pawlak, R., Correa, A., Bell-Pedersen, D. & Ebbole,
D. J. The Neurospora crassa pheromone precursor genes are regulated by the mating
type locus and the circadian clock. Mol Microbiol 45, 795-804 (2002).

| | | | | | % Identity | | |
|---|---|---|---|---|---|---|---|
| | | | | SEQ | Full | Res. | Res. |
| | Code | Species | Mature Peptide ligand | ID NO: | Sc.Ste2 | 289-296 | 228-248 |
| 1 | Sc¹ | Saccharomyces | 1-WHWLQLKPGQPMY | 1 | 100 | 100 | 100 |
| | | cerevisiae | | | | | |
| 2 | Sca² | Saccharomyces | 1-NWHWLRLDPGQPLY | 2 | 67.68 | 100 | 100 |
| | | cerevisiae | | | | | |
| 3 | Vp2² | Vanderwaltozyma | 1--WHWLRLRYGEPIY | 3 | 52.82 | 100 | 90.48 |
| | | polyspora2 | 2-PWHWLRLRYGEPIY | 4 | | | |
| 4 | Vp1² | Vanderwaltozyma | 1-WHWLELDNGQPIY | 5 | 50.79 | 100 | 85.71 |
| | | polyspora1 | | | | | |
| 5 | Td | Torulaspora | 1-GWMRLRLGQPL | 6 | 49.8 | 100 | 95.24 |
| | | delbrueckii | 2-GWMRLRLGQPM | 7 | | | |
| | | | 3-GWMRLRIGQPL | 8 | | | |
| 6 | Sk³ | Saccharomyces | 1--WHWLSFSKGEPMY | 9 | 49.3 | 100 | 90.48 |
| | | kluyveri | 2-PWHWLSFSKGEPMY | 10 | | | |
| 7 | Kl² | Kluyveromyces | 1---WSWITLRPGQPIF | 11 | 48.93 | 75.0 | 85.71 |
| | | lactis | 2-SPWSWITLRPGQPIF | 12 | | | |
| 8 | Zr² | Zygosaccharomyces | 1--HFIELDPGQPFM | 13 | 44.92 | 100 | 100 |
| | | rouxii | 2-AHFIELDPGQPMF | 14 | | | |
| 9 | Zb | Zygosaccharomyces | 1--HLVRLSPGAAMF | 15 | 44.34 | 100 | 100 |
| | | bailii | 2--PLVRLSPGAAMF | 16 | | | |
| | | | 3-APLVRLSPGAAMF | 17 | | | |
| | | | 4-AHLVRLSPGAAMF | 18 | | | |
| 10 | Cg⁴ | Candida glabrata | 1-WHWVRLRKGQGLF | 19 | 43.45 | 87.5 | 80.95 |
| | | | 2-WHWVKIRKGQGLF | 20 | | | |
| 11 | Ag | Ashbya gossypii | 1-WFRLSLHHGQSM | 21 | 41.04 | 87.5 | 80.95 |
| 12 | Ss | Scheffersomyces | 1--WHWTSYGVFEPG | 22 | 36.22 | 75.0 | 66.67 |
| | | stipitis | 2-PWHWTSYGVFEPG | 23 | | | |
| 13 | Kp | Komagataella | 1-FRWRNNEKNQPGF | 24 | 35 | 87.5 | 66.67 |
| | | (Pichia) pastoris | | | | | |
| 14 | Cgu² | Candida (Pichia) | 1-KKNSRFLTYWFFQPIM | 25 | 33.9 | 87.5 | 66.67 |
| | | guilliermondii | | | | | |
| 15 | Cp² | Candida | 1-KPHWTTYGYYEPQ | 26 | 31.33 | 87.5 | 80.95 |
| | | parapsilosis | | | | | |
| 16 | Cau | Candida auris | 1-KWGWLRFFPGEPFV | 27 | 30.87 | 87.5 | 71.43 |
| 17 | Yl² | Yarrowia | 1-WRWFLWLPGYGEPNW | 28 | 30.8 | 87.5 | 38.10 |
| | | lipolytica | | | | | |
| 18 | Cl² | Candida | 1--KWKWIKFRNTDVIG | 29 | 30.69 | 75.0 | 71.43 |
| | | (Clavispora) | 2---WGWIHFLNTDVIG | 30 | | | |
| | | lusitaniae | 3-PKWKWIKFRNTDVIG | 31 | | | |
| 19 | Ca⁵ | Candida albicans | 1-GFRLTNFGYFEPG | 32 | 28.83 | 87.5 | 85.71 |
| 20 | Ct² | Candida tropicalis | 1-KFKFRLTRYGWFSPN | 33 | 28.11 | 75.00 | 76.19 |
| 21 | Cn | Candida tenuis | 1-FSWNYRLKWQPIS | 34 | 27.49 | 62.5 | 71.43 |
| 22 | Le² | Lodderomyces | 1----WMWTRYGRFSPV | 35 | 26.97 | 87.5 | 76.19 |
| | | elongisporous | 2-DPGWMWTRYGRFSPV | 36 | | | |
| 23 | Gc | Geotrichum | 1--GDWGWFWYVPRPGDPAM | 37 | 26.76 | 87.5 | 57.14 |
| | | candidum | 2-PGDWGWFWYVPRPGDPAM | 38 | | | |
| 24 | Bm | Baudoinia | 1-GWIGRCGVPGSSC | 39 | 26.56 | 87.5 | 42.86 |
| | | compniacensis | | | | | |
| 25 | So² | Schizosaccharomyces | 1-----TYEDFLRVYKNWWSFQNPDRPDL | 40 | 26.04 | 87.5 | 28.57 |
| | | octosporus | 2-PACTTYEDFLRVYKNWWSFQNPDRPDL | 41 | | | |
| 26 | Tm | Tuber melanosporum | 1-WTPRPGRGAY | 42 | 25.94 | 100 | 38.10 |
| 27 | Ao² | Aspergillus oryzae | 1-WCALPGQGC | 43 | 24.67 | 87.5 | 33.33 |
| 28 | Sp⁶ | Schizosaccharomyces | 1--TYADFLRAYQSWNTFVNPDRPNL | 44 | 23.75 | 87.5 | 28.57 |
| | | pombe | 2-KTYADFLRAYQSWNTFVNPDRPNL | 45 | | | |
| 29 | Af² | Aspergillus | 1-WCHLPGQGC | 46 | 23.67 | 87.5 | 42.86 |
| | | (Neosartorya) | | | | | |
| | | fischeri | | | | | |
| 30 | Pd | Pseudogymnoascus | 1---FCWRPGQPCG | 47 | 23.56 | 87.5 | 28.57 |
| | | destructans | 2---FCQRPGQLCG | 48 | | | |
| | | | 3-LEFGGLEKEQNS | 49 | | | |
| 31 | Sj² | Schizosaccharomyces | 1-----VSDRVKQMLSHWWNFRNPDTANL | 50 | 23.3 | 87.5 | 28.57 |
| | | japonicus | 2-PERRVSDRVKQMLSHWWNFRNPDTANL | 51 | | | |
| 32 | Pb⁷ | Paracoccidioides | 1-WCTRPGQGC | 52 | 22.9 | 87.5 | 28.57 |
| | | brasiliensis | | | | | |
| 33 | Mg | Mycosphaerella | 1-GNSFVGWCGAIGAPCA | 53 | 22.44 | 100 | 42.86 |
| | | graminicola | 2-------WCGAIGAPCA | 54 | | | |
| 34 | Pr | Penicillium | 1-WCGHIGQGC | 55 | 21.81 | 87.5 | 33.33 |
| | | chrysogenum | 2-KWCGHIGQGC | 56 | | | |
| 35 | An⁸ | Aspergillus | 1-WCRFRGQVCG | 57 | 21.73 | 87.5 | 38.10 |
| | | nidulans | | | | | |
| 36 | Sn² | Phaeosphaeria | 1-KYNGWRYRPYGLPVG | 58 | 21.61 | 75.0 | 38.10 |
| | | nodorum | | | | | |
| 37 | Hj | Hypocrea jecorina | 1-WCYEIGEPCW | 59 | 19.87 | 75.0 | 15.00 |
| | | | 2-WCWILGGKCW | 60 | | | |
| 38 | Bc² | Botrytis cinerea | 1-WCGRPGQPC | 61 | 19.54 | 75.0 | 28.57 |
| 39 | Bb | Beauvaria bassiana | 1-WCMRPGQPCW | 62 | 19.23 | 50.0 | 15.00 |
| | | | 2-WCMQTPKCW | 63 | | | |
| 40 | Nc⁹ | Neurospora crassa | 1-QWCR---IHGQSCW | 64 | 18.94 | 50.0 | 20.00 |
| | | | 2-QVCNMRLHPKKVCW | 65 | | | |
| 41 | She | Sporothrix | 1---YCPLKGQSCW | 66 | 18 | 62.5 | 15.00 |
| | | scheckii | 2-QRYCPLKGQSCW | 67 | | | |
| 42 | Mo² | Magnaporthe oryzea | 1-QWCPRRGQPCW | 68 | 17.56 | 50.0 | 20.00 |
| 43 | Dh | Dactylellina | 1-WCVYNSCP | 69 | 17.02 | 37.5 | 33.33 |
| | | haptotyla | | | | | |
| 44 | Fg² | Fusarium | 1-WCWWKGQPCW | 70 | 16.8 | 50.0 | 30.00 |
| | | graminearum | 2-WCTWKGQPCW | 71 | | | |
| 45 | Cc | Capronia coronata | 1-GLSYWKGVNDGGSS | 72 | 16.05 | 50.0 | 19.05 |

TABLE 4
Annotated pre-pro peptides used to infer mature peptide ligand sequences.

| | | Mature peptide | | SEQ |
|---|---|---|---|---|
| | Code | ligand | Precursor | ID NO: |
| 1 | Af² | 1-WCHLPGQGC | MRLLSLVLATFAATAVQADITPWCHLPGQG | 73 |
| | | | CYMLKRAADASDEVRRSASAVAEAVAEAFP | |
| | | | QTPWCHLPGQGCAKAKRAAEAAEEVKRSAD | |
| | | | AFAEAMAAFEKE | |
| 2 | Ag | 1-WFRLSLHHGQSM | MKTTHILSLATLAACAPVQPAPVQPTDLAA | 74 |
| | | | AANVPEKAVLGFFQLYNVGDVELLPVDDGA | |
| | | | HSGILFVNRTLADVDYSSEHVVQKWFRLSL | |
| | | | HHGQSM | |
| 3 | An⁸ | 1-WCRFRGQVCG | MKLFFVSILLAALLATAVKAAPAAELQHRW | 75 |
| | | | CRFAGRICPPTKRTADALNFVKREAEAVAE | |
| | | | PFKINRWCRFRGQVCGKAKRAAEAIGNVKL | |
| | | | SAEAVADAMAFLDELTREEYAQLAKDFGHL | |
| | | | KESDNSDG | |
| 4 | Ao² | 1-WCALPGQGC | MKLISVVVAALAATSVQAGVLQKWCSLPAQ | 76 |
| | | | GCYMLKRAADASGDVRRSAEALSEAMPDAE | |
| | | | ALAKWCALPGQGCLKAKRAAEAVEEARRSA | |
| | | | DALADAMADLGEY | |
| 5 | Bb | 1-WCMRPGQPCW | MKLSLVMLATAATTVIAAPRPWCMRPGQPC | 77 |
| | | 2-WCMQT-PKCW | WKLKRAVDALGEPAPSPVEPLDADNIGLFA | |
| | | | SGAHDRLLHLASSDAANVDDEGAFEKRWCM | |
| | | | QTPKCWKLLADEDGELSKRWCMRPGQPCWK | |
| | | | RSVDEHGDLAKRWCMRPGQPCWKAKRAAES | |
| | | | VLNAGQEDGDAQEQDCGDDGECSVAKRHLD | |
| | | | GLHHVARAIVEAF | |
| 6 | Bc² | 1-WCGRPGQPC | MKFTNAIALAILAATATAVAVPEPWCGRPG | 78 |
| | | | 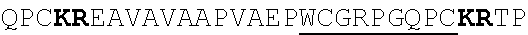 | |
| | | | 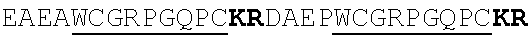 | |
| | | | EALPEAWCGRPGQPCKRTPLAEAEAEAWCG | |
| | | | RPGQPCRKNKRAAEAVAEAFAEPWCGRPGQ | |
| | | | PCKRDAEADVSEAAIKRCNMVGGACFEAKR | |
| | | | LARDLAEATAETVEDSDLFLRSLNIETREV | |
| | | | SEVVAREAEAWCGRPGQPCKRDAEAWCGRP | |
| | | | GQPCKREALAEAEAWCGRPGQPCKREALAE | |
| | | | AEAWCGRPGQPCKRTAEPWCGRPGQPCKEK | |
| | | | READPEAEAWCGRPGQPCRAVKRAAEAIAE | |
| | | | ALAEPTAEAWCGRPGQPCKREALAEAEANA | |
| | | | EAWCGRPGQPCRKAKRDAFALAYAADVALA | |
| | | | QL | |
| 7 | Bm | 1- | MKFSIVAVAAVAAQAAAVSGSTSAVFKDGV | 79 |
| | | GWIGRCGVPGSSC | GACNVPGQKCHTVKNAARDILNAINKPTDV | |
| | | | DDQQSYFCDIQGSAGCNQLHGSVDKLQQAA | |
| | | | IKAYHTVAAREAEAEAEAEANPGYGWIGRC | |
| | | | GVPGSSCNKKREADPGYGWIGRCGVPGSSC | |
| | | | NKKRDEDAAAREHWLAQREAGGWIGRCGVP | |
| | | | GSSCNKKREEEVEVLRREAEAGGWIGRCGV | |
| | | | PGSSCNKARDANPGGWIGRCGVPGSSCNKK | |
| | | | REAGGWIGRCGVPGSSCNKARDAEDDQKIQ | |
| | | | QMQDAIRAFNPEIEKAECNQDGQPCDLIKT | |
| | | | AAQALHNNTRREAEAGGWIGRCGVPGSSCN | |
| | | | KNKRALAFCQSGENCTGPAYAHLQSQDATA | |
| | | | DKAEKDCHGPNGACTIAARALAELEQAVDA | |
| | | | ALLDADA | |
| 8 | Ca⁵ | 1- | MKFSLTLLTATIATIVAAAPAQYTGQAIDS | 80 |
| | | GFRLTNFGYFEPG | NQVVEIPESAVEAYFPIDDELTPVFGEIDN | |
| | | | KPVILIVNGTTLTSGANNEKREAKSKGGFR | |
| | | | LTNFGYFEPGKRDANADAGFRLTNFGYFEP | |
| | | | GKRDANAEAGFRLTNFGYFEPGK | |
| 9 | Cau | 1- | MKFSITAIIAATGSLVAAAPTPSSTDAPSF | 81 |
| | | KWGWLRFFPGEPFV | SEVPSSVESSFGVPTEAIIGQFSFDADEYP | |
| | | | LLTVYEDRRYIILLNSTIMEEAYASLNSGN | |
| | | | EKRDAEAEAKWGWLRFFPGEPFVKRDAEAD | |
| | | | AEAKWGWLRFFPGEPFVKRDAEADAEAKWG | |
| | | | WLRFFPGEPFVKRDAEADAEAKWGWLRFFP | |
| | | | GEPFVKRDADAEAKWGWLRFYPGEPFVKRE | |
| | | | VEADLEG | |
| 10 | Cc | 1- | MHISSTTVTLVLTASFIQSALAFPVPAFLD | 82 |
| | | 2--- | VLRRDASPDPRLSYWKGVNDGGSSKIKSRR | |
| | | SYWKGVNDGGSS | WLSPIIEMLDKREPGLSYWKGVNDGGSSKR | |
| | | | EAAPEPDPGLSYWKGVNDGGFSKREAEPEP | |
| | | | EPEPRLPYWKGVNDGGSSKREAAPEPDPGL | |
| | | | SYWKGVNDGGSSKREAAPEPEPEPEPGLSY | |
| | | | WKGVNDGGSSKRGLSYWKGVNDGGSSKREA | |
| | | | EPEPQPDALPALGLT | |
| 11 | Cg⁴ | 1- | MRFLRFISTVALLITGLATAQPVGEELGET | 83 |
| | | WHWVRLRKGQGLF | VEVPSEAFIGYLDFGATNDVAILPISNKTN | |
| | | 2- | NGLLFVNTTLYNQATKGEKLSDFTKRDANP | |
| | | WHWVKIRKGQGLF | DAEAEAWHWVKIRKGQGLFRRSADASPEAE | |
| | | | AWHWVRLRKGQGLFRRSADASPEAEAWHWV | |
| | | | RLRKGQGLF | |
| 12 | Cgu² | 1- | MKFSTAFVSTLFATYAAAAPLAAASDKIPV | 84 |
| | | KKNSRFLTYWFFQP | PFPKSAVNQIVTIDETNAPIYLNNSGTITL | |
| | | IM | FLVNTTVKEESPEKRELGEVATGYEFNAAQ | |
| | | | YMKRESFPIENLVPESSLEKREDKKNSRFL | |
| | | | TYWFFQPIMKRGEEETSEVVKREAKKNSRF | |
| | | | LTYWFFQPIMKREEDIVAGDEMVKREAKKN | |
| | | | SRFLTYWFFQPIMKREGGNEVEKRDAKKNS | |
| | | | RFLTYWFFQPIM | |
| 13 | Cl² | 1-- | MKFSLAIIFSLAAAVVSAAPVAPESSSDFQ | 85 |
| | | KWKWIKFRNTDVIG | IPEEAIISSQALGDDQLPLLLGEGNATYFV | |
| | | 2--- | LVNGTTLAEAYGITKRDAEAFDATYLGSSV | |
| | | WGWIHFLNTDVIG | 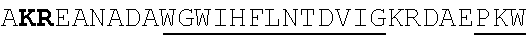 | |
| | | 3- | 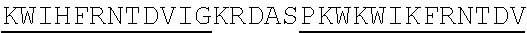 | |
| | | PKWKWIKFRNTDVI | 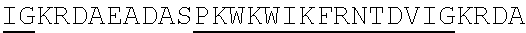 | |
| | | G | 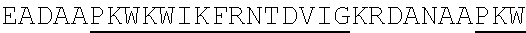 | |
| | | | RWINFRNTDVIGKREAQE | |
| 14 | Cn | 1- | MRLSTILTLALTSKFVFSAPVEKVKREDGL | 86 |
| | | FSWNYRLKWQPIS | DVPDEAIIAVYPIDEYKQPFYAEADGQNYV | |
| | | | VILNTTALGEADLAKRDADAFSWNYRLKWQ | |
| | | | PISKRDADADADADAFSWNYRLKWQPISKR | |
| | | | DADADADADADAFSWNYRLKWQPIS | |
| 15 | Cp² | 1- | MKFSIAVLTAIAAALVASAPVASKEAEVPA | 87 |
| | | KPHWTTYGYYEPQ | LPVDNVLERVVEAFFNGPSIDAEIKDKTAA | |
| | | | DVKGVVGSQKREAEAKPHWTTYGYYEPQKR | |
| | | | DANAEAEAKPHWTTYGYYEPQKRDANAEAE | |
| | | | AKPHWTTYGYYEPQK | |
| 16 | Ct² | 1- | MKFSLALLTTVAAALVVAAPTQAPVEEAEV | 88 |
| | | KFKFRLTRYGWFSP | PTNETGLAIPDSAVCAIVPLDGELAPVFVE | |
| | | N | LDDIPVLMIVNTTAVEEAYQAEEEAYEAEE | |
| | | | GSSDVEKRDAAKFKFRLTRYGWFSPNKREE | |
| | | | IDAEDIIDAEKRDAAKFKFRLTRYGWFSPN | |
| | | | KRDIGDEEDIVDAEKRDAAKFKFRLTRYGW | |
| | | | FSPNKRELAEEEETVDAEKRDAAKFKFRLT | |
| | | | RYGWFSPNKREVAEENDIVEKRDAAKFKFR | |
| | | | LTRYGWFSPN | |
| 17 | Dh | 1-WCVYNSCP | MQLKHTITILSLLAPLLNALPVAEPEPTAA | 89 |
| | | 2-WCVYNSCPKT | PEAKAGSGDVMLPRSWCIYNSCPKNKRAPE | |
| | | | PVAEPVAIPEPTAAPEPVIPAHIEARGVEA | |
| | | | VRRWCVYNSCPKTKREAAPAPEPTAEPEPV | |
| | | | IPAHIEARGEEYVKRWCVYNSCPKTKRAAE | |
| | | | PIPEPTAQPEPIIPDHVQAQGEEFVKRWCV | |
| | | | YNSCPKTKREAQPEPTAAPEPVIPDHIQAR | |
| | | | GEEYIKRWCVYNSCPKTKREAQPEPTAAAE | |
| | | | AGIPAHIQARGEEYVKRWCVYNSCPKTKRE | |
| | | | AMPEPTAAPEPVIPDHIQARGEEFVKRWCV | |
| | | | YNSCPKTKREAAPAPAPTAAPEPVIPAHIQ | |
| | | | ARGEEYVKRWCVYNSCPKTKREALPAPTAA | |
| | | | PEPIPAPEAEKMEPRSWCIYNSCPKYKRAA | |
| | | | QPVPEPTAMPVA | |
| 18 | Fg² | 1-WCWWKGQPCW | MKYSILTLAAVASTTLAVAVPAPQPDPVAE | 90 |
| | | 2-WCTWKGQPCW | PMPWCTWKGQPCWKEKMARREAQPEPEAVA | |
| | | | APEPDPVAEPMPWCTWKGQPCWKEKMARRA | |
| | | | AQPEPEAVAAPEPDPVAEPMPWCTWKGQPC | |
| | | | WKEKMKMAKREAQPEPEAVAAPEPDPVAEP | |
| | | | MPWCTWKGQPCWKEKMAKRAAEAEAEPEPI | |
| | | | PAPQPDPVAEAEPWCTWKGQPCWKAKMAKR | |
| | | | AAEAEAEAEPIPDPVAAPQPDPVAEPMPWC | |
| | | | TWKGQPCWKEKMAKREAKPEPWCWWKGQPC | |
| | | | WKAKRDAAPEPWCWWKGQPCWKAKRNAAPE | |
| | | | PMPEPANEPRWCWWKGQPCWKSKSKRDASP | |
| | | | EPWCWWKGQPCWKAKRDAGEALTVALHATR | |
| | | | GVETRSVAETEHLPRDAAHQAKRSIVELAN | |
| | | | VIALSARGSPEEYFKHLYLEEFFPEIPHNA | |
| | | | TAKRDVKTLQEDKRWCWWKGQPCWKAKRAA | |
| | | | EAVLHAVDGSDGAGAPGGPEEHFDTSHFNP | |
| | | | QNFEAKRDLMAIKAAARSVVESLEG | |
| 19 | Gc | 1-- | MRFSLATVYAFTVIGTVLGVPIASSEPTAT | 91 |
| | | GDWGWFWYVPRPGD | TLSTVAAASATFSPGGDSPFTGIKNFPDFA | |
| | | PAM | 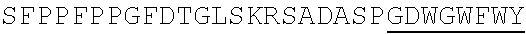 | |
| | | 2- | 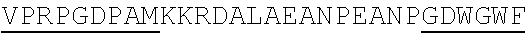 | |
| | | PGDWGWFWYVPRPG | WYVPRPGDPAMKKRDALADANPDANPVE | |
| | | DPAM | | |
| 20 | Hj | 1-WCYRIGEPCW | METKEKTVVPKSKSPLSIYFSLDRVSLHPS | 92 |
| | | 2-WCWILGGKCW | SLLISPSPSHLLSPSPHIAKLQTMKFLAAV | |
| | | | TVFASAALAAPNPEPWCYRIGEPCWKLKRT | |
| | | | AEAFNLAVRSHDLTTRAQGEAIPDEVALSA | |
| | | | IEGLDQLKKLILVSTEDPSSLLPPNATEPE | |
| | | | SKRDVEVEEDKRWCYRIGEPCWKAKREAEA | |
| | | | EAAAEEEKRWCYRIGEPCWKAKRTDEISEE | |
| | | | KRWCWILGGKCWKTKRVAEAVLSATIEGDE | |
| | | | KRSVEAEGNADEKRWCYRIGEPCWKAKRDL | |
| | | | ETIQDVARSVIESMQ | |
| 21 | Kl² | 1--- | MKFSTILAASTALISVVMAAPVSTETDIDD | 93 |
| | | WSWITLRPGQPIF | LPISVPEEALIGFIDLTGDEVSLLPVNNGT | |
| | | 2- | 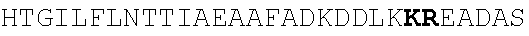 | |
| | | SPWSWITLRPGQPI | 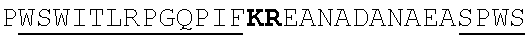 | |
| | | F | 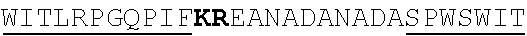 | |
| | | | LRPGQPIFKREANPEAEADAKPSAWSWITL | |
| | | | RPGQPIF | |
| 22 | Kp | 1- | MKSLILNIISVTLAITSTAASAPVESIFAN | 94 |
| | | FRWRNNEKNQPFG | QPDSSLTDTNDGVGVGMSTIKEEDFGKHFV | |
| | | | ENQILDEAVIMSLKLRKGVNLFFLDDICLA | |
| | | | TELIGNKIAQIEATDLSERLAQSWTNIRKN | |
| | | | RLFGKREAEAEAEAEAFRWRNNEKNQPFGK | |
| | | | REAEAEAEAEAEAEAEAEAFRWRNNEKNQP | |
| | | | FGKREAEAEAEAEAEAEAEAFRWRNNEKNQ | |
| | | | PFGKREAEAEAEAEAFRWRNNEKNQPFGKR | |
| | | | EADAEAEAEAEAFRWRNNEKNQPFGKREAE | |
| | | | AEAEAEAFRWRNNEKNQPFGKREAEAEAEA | |
| | | | EAEAEAFRWRNNEKNQPFGKREAEAEAEAE | |
| | | | AFRWRNNEKNQPFGKREADAEAEAEAEAFR | |
| | | | WRNNEKNQPFGKREASIDTGTDDGAYWSWR | |
| | | | KNSVLERQ | |
| 23 | Le² | 1---- | MKFSTAVLTAIAVTLVAAAPVDIDTNANAA | 95 |
| | | WMWTRYGRFSPV | DNVIEATTSNEEAAIPETTEIALDNAEQIT | |
| | | 2- | DEQIPSDCGLELGPETQIEGELPQEDGEEG | |
| | | DPGWMWTRYGRFSP | 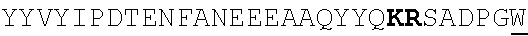 | |
| | | V | 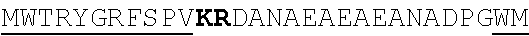 | |
| | | | 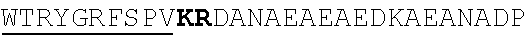 | |
| | | | 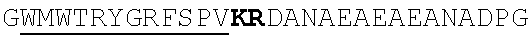 | |
| | | | 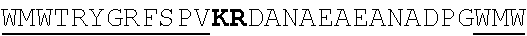 | |
| | | | 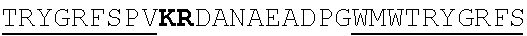 | |
| | | | 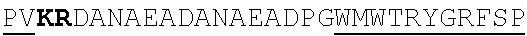 | |
| | | | 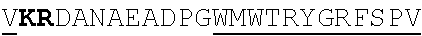 | |
| 24 | Mg | 1- | MKLAVSTVLMVAVTLTQALAVADAEPKRRR | 96 |
| | | GNSFVGWCGAIGAP | GNSFVGWCGAIGAPCAKVKRDAEAMPDPKK | |
| | | CA | 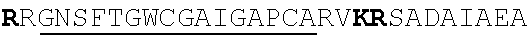 | |
| | | 2------- | 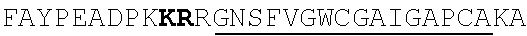 | |
| | | WCGAIGAPCA | KRDIIEVGESVEEAVHDVYAREAEAEADPK | |
| | | | 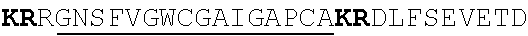 | |
| | | | VSAEDSEDEDAIYARDAAPEARRKKKAKKP | |
| | | | 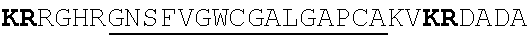 | |
| | | | 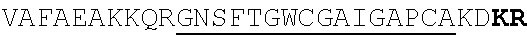 | |
| | | | EEHEILKTDVCEADDGECKALRNAYEAFHE | |
| | | | IKARDAELEAENLASIDDDDELTKREVEVC | |
| | | | NEPDGECDLAKRALDTIEAKLDAAIKAL | |
| 25 | Mo² | 1-QWCPRRGQPCW | 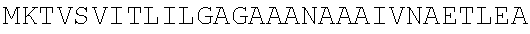 | 97 |
| | | | 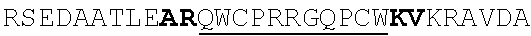 | |
| | | | FASAMHSNEARDVATTTSPSDGHLTARDLS | |
| | | | HLPGGAAYNAKRSVNALAALLASTQYDPEA | |
| | | | FYNDLYLDRYFDPDTSVDAKAVDEKPDAEA | |
| | | | KTEKRDEEGGHLEARQWCPRRGQPCWKRDV | |
| | | | EHDKRHCNSAGEACDVAKRAVGALLSAVED | |
| | | | SGADLAKRQWCPRRGQPCWKRDNVFEPVAL | |
| | | | GRRDVSDAEADVLTKRQWCPRRGQPCWKRS | |
| | | | EISGLEARCYGPAGECTKAQRDLNAIHLAA | |
| | | | RDVLASLDFGRHLSSRLLDHS | |
| 26 | Nc⁹ | 1-QWCRI--- | MKFTLPLVIFAAVASATPVAQPNAEAEAQW | 98 |
| | | HGQSCW | CRIHGQSCWKVKRVADAFANAIQGMGGLPP | |
| | | 2- | RDESGHQPAQVAKRQVDELAGIIALTQEDV | |
| | | QVCNMRLHPKKVCW | NAYYDSLSLQEKFAPSTEEEKKTEKVAKRE | |
| | | | AEAEAQWCRIHGQSCWKKREAEAQWCRIHG | |
| | | | QSCWKRDALPEAEPQWCRIHGQSCWKKRDA | |
| | | | APEAAPEAEANPQWCRIHGQSCWKAKRAAE | |
| | | | AVMTAIQSAEAESALLLRDTTFSPVDRVGK | |
| | | | RDPQVCNMRLHPKKVCWKRDASPEAACNAP | |
| | | | DGSCTKATRDLHAMYNVARAILTAHSDEN | |
| 27 | Pb⁷ | | | |
| 28 | Pd | 1---FCWRPGQPCG | MKYLATLCVAALVAGVNSAAIAAAEPFCWR | 99 |
| | | 2---FCQRPGQLCG | LGQPCDKVKRAAEAFAEAFDEPIAEAEAFD | |
| | | 3-LEFGGLEKEQNS | EPIAEAEASAFCWRPGQICEKAKRAALALA | |
| | | | HTVADANPEAEAFFDKLAIDEAFPEPEAVA | |
| | | | DAEIADKVKREAEAEAFCWRPGQPCGKVKR | |
| | | | AADAIASALAEPAPEPFCQRPGQLCGKVKR | |
| | | | DAEAVAEAFCWRPGQPCGKAKREANALAEA | |
| | | | AAEALEFGGLEKEQNSKRIFRPPHYTTTAI | |
| | | | FPTDPRLFHHFHEEQPYDCRKVDPNCVTVE | |
| | | | A | |
| 29 | Pr | 1--WCGHIGQGC | 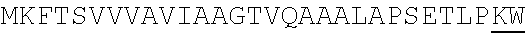 | 100 |
| | | 2-KWCGHIGQGC | CGHIGQGCKRTTDASLDVKRSADALAEAMA | |
| | | | 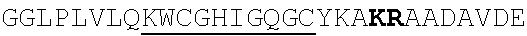 | |
| | | | VKRTSDALARAFAALEEEDDE | |
| 30 | Sc¹ | 1- | MRFPSIFTAVLFAASSALAAPVNTTTEDET | 101 |
| | | WHWLQLKPGQPMY | AQIPAEAVIGYLDLEGDFDVAVLPFSNSTN | |
| | | | NGLLFINTTIASIAAKEEGVSLDKREAEAW | |
| | | | HWLQLKPGQPMYKREAEAEAWHWLQLKPGQ | |
| | | | PMYKREADAEAWHWLQLKPGQPMY | |
| 31 | Sca² | 1- | MKLSALLSTVALASTSFAAPIDTTASNENL | 102 |
| | | NWHWLRLDPGQPLY | NSTDIPAEAVIGYLDLGSDSDVAMLPFQNS | |
| | | | TSNGLLFVNTTIVQQAAQENDDSVGLAKRE | |
| | | | ANAEAGWHWLRLDPGQPLYKREADADAEAN | |
| | | | WHWLRLDPGQPLYKREAEADAEANWHWLRL | |
| | | | DPGQPLYKREADADAEANWHWLRLDPGQPL | |
| | | | YKREADADAEANWHWLRLDPGQPLY | |
| 32 | She | 1---YCPLKGQSCW | MKTAAVFTILAVGASAAAVAEAEAYCQSVG | 103 |
| | | 2-QRYCPLKGQSCW | QSCYQVKRAAEAFAEAIADLGAPEAGISRR | |
| | | | SLSFGGVHNNAIRAIDGLASIVASTQYNPR | |
| | | | SFYSDLSLESHFPVPVEEPVTKREAEADAD | |
| | | | 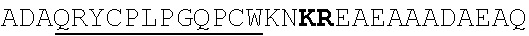 | |
| | | | 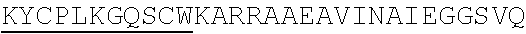 | |
| | | | 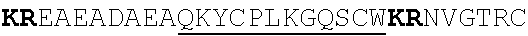 | |
| | | | YAPGGACANASRDLHAIYNAARSVIESLPK | |
| | | | AE | |
| 33 | Sj² | 1----- | MKFSAIFILSLFASAFAAPVPSSDAVEAAA | 104 |
| | | VSDRVKQMLSHWWN | PIIPELLSTEQVVLEGRVSDRVKQMLSHWW | |
| | | FRNPDTANL | 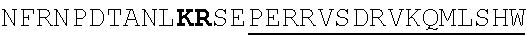 | |
| | | 2- | 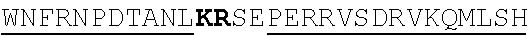 | |
| | | PERRVSDRVKQMLS | 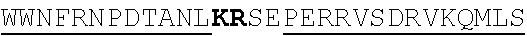 | |
| | | HWWNFRNPDTANL | HWWNFRNPDTANLKKRALTDAQEEEAESEM | |
| | | | DLLSYLLYSNDTSIAASGLNATEMVETILK | |
| | | | DYE | |
| 34 | Sk³ | 1-- | MKLFTTLSASLIFIHSLGSTRAAPVTGDES | 105 |
| | | WHWLSFSKGEPMY | SVEIPEESLIGFLDLAGDDISVFPVSNETH | |
| | | 2- | YGLMLVNSTIVNLARSESANFKGKREADAE | |
| | | PWHWLSFSKGEPMY | 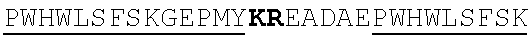 | |
| | | | GEPMY | |
| 35 | Sn² | 1- | MRFNAVIAACILAVTVSGAALPTEDAAITD | 106 |
| | | KYNGWRYRPYGLPV | AATITTTEAEITEAEIIKAAPEEDDFFDDD | |
| | | G | EQFEKRDAASWKYNGWRYRPYGLPVGKRDA | |
| | | 2- | DAEAGWRYRPYGLPVGKREAAPEADAEAKY | |
| | | GWRYRPYGLPVG | NGWRYRPYGLPVGKREAEAKYNGWRYRPYG | |
| | | | LPVGKREAEADASAEARYNGWRYRPYGLPV | |
| | | | GR | |
| 36 | So² | 1----- | MKFFSLVALLFALASAAPIPATSKDSGVSP | 107 |
| | | TYEDFLRVYKNWWS | LDQLPSKTYEDFLRVYKNWQTFQNPDRPDL | |
| | | FQNPDRPDL | KKRDVPELPSKTYEDFLRVYKNWWSFQNPD | |
| | | 2- | 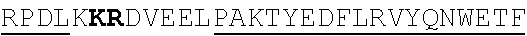 | |
| | | PACTTYEDFLRVYK | QNPDRPDLKKRDVPELPSKTYEDFLRVYKN | |
| | | NWWSFQNPDRPDL | 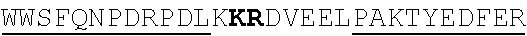 | |
| | | | VYQNWETFQNPDRPDLKKRDVPELPSKTYE | |
| | | | DFLRVYKNWWSFQNPDRPDLKKRDVPELPS | |
| | | | KTYEDFLRVYKNWWSFQNPDRPDLKKRDVE | |
| | | | EPVLKTEKDKEDYYHFLEFYVMNVPFNSTV | |
| | | | AQTNISSHFD | |
| 37 | Sp⁶ | 1-- | MKITAVIALLFSLAAASPIPVADPGVVSVS | 108 |
| | | TYADFLRAYQSWNT | KSYADFLRVYQSWNTFANPDRPNLKKREFE | |
| | | FVNPDRPNL | 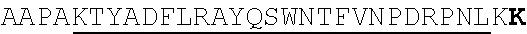 | |
| | | 2- | 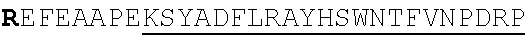 | |
| | | KTYADFLRAYQSWN | 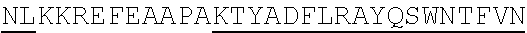 | |
| | | TFVNPDRPNL | PDRPNLKKRTEEDEENEEEDEEYYRFLQFY | |
| | | | IMTVPENSTITDVNITAKFES | |
| 38 | Ss | 1-- | MHLRSTAILSAVVFTSVALSAPTSGQNIDI | 109 |
| | | WHWTSYGVFEPG | DFPDESIAGAIPLSYDLVPIIGSYQGQNVI | |
| | | 2- | LIVNSTIAAASEAAASEGKSKRDANAWHWT | |
| | | PWHWTSYGVFEPG | 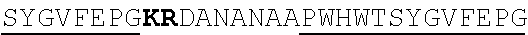 | |
| | | | 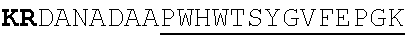 | |
| 39 | Td | 1-GWMRLRLGQPL- | MKFFNTILSTTLFTYVALAAPVESDPVNIP | 110 |
| | | | SEAILGYMDFTEDQDVGVVAYTNSTFSGLI | |
| | | | FFNSSIIETKDLTKRDAEAGWMRLRLGQPL | |
| | | | 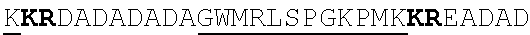 | |
| | | | AEAGWMRLRIGQPL | |
| 40 | Tm | 1-WTPRPGRGAY | MKVTILFLATLLSAALSEPIPWEVNGNRGV | 111 |
| | | | YRREPEAEAEAWHPRAGDPMAIWQKRNAEP | |
| | | | YPEAEPEAIPWTPRPGRGAYRRHARPWTPR | |
| | | | PGRGAYRRSAEAWHPRAGPPAYTLSKRDAA | |
| | | | PEPVRFQPIGSFYKE | |
| 41 | Vp1² | 1- | MKLTNVLSAVALASTALAAPVAKDATNTTD | 112 |
| | | WHWLELDNGQPIY | ASSVQIPAEAVIGYLDLEQSNDVAMLQFSN | |
| | | | STNNGILFVNSTILKAAYAEANANSNSNTK | |
| | | | REAKADAWHWLELDNGQPIYKREANAEAKP | |
| | | | WHWLELDNGQPIYKREAKAEAKADAWHWLE | |
| | | | LDNGQPIYKREAKAEAKADAWHWLELDNGQ | |
| | | | PIYKREAEAKAGAWHWLELDNGQPIY | |
| 42 | Vp2² | 1-- | MKFSTVLSTVALAATAVSAAPISRASNETV | 113 |
| | | WHWLRLRYGEPIY | ESVESGLNVPAEAVLGYLDFGEKDDVAMLP | |
| | | 2- | FSNGTSNGLLFVNTTIYDAAFADSDDESAS | |
| | | PWHWLRLRYGEPIY | LAKRDAEAWHWLRLRYGEPIYKREDSEGVE | |
| | | | 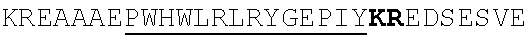 | |
| | | | 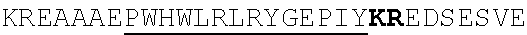 | |
| | | | KREANADADAWHWLRLRYGEPIY | |
| 43 | Yl² | 1- | MKFSTIALAAVACLVSAAPAAPVGTGSHGP | 114 |
| | | WRWFWLPGYGEPNW | QSIPEEAIVGGLQGTENEIFVFFNDDESGK | |
| | | | QGIAIIDAKKAQEAGFMDPQPDSEVAAGNA | |
| | | | KREASPEAWRWFWLPGYGEPNWKRDAMPAD | |
| | | | MDKEKREANPEAWRWFWLPGYGEPNWKRDA | |
| | | | MPADMDKEKREANPEAWRWFWLPGYGEPNW | |
| | | | KRDAMPADMDKEKREANPEAWRWFWLPGYG | |
| | | | EPNW | |
| 44 | Zb | 1-- | MRFSITLCSTLCALTVAAAPIEEYKRAPVA | 115 |
| | | HLVRLSPGAAMF | 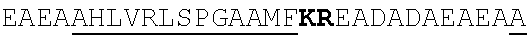 | |
| | | 2-- | 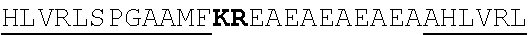 | |
| | | PLVRLSPGAAMF | SPGAAMFKREAEADADAEAEAAPLVRLSPG | |
| | | 3- | 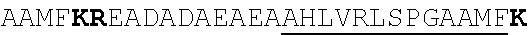 | |
| | | APLRLSPGAAMF | 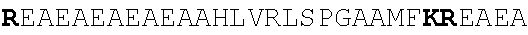 | |
| | | 4- | 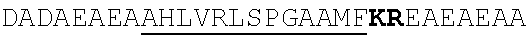 | |
| | | AHLVRLSPGAAMF | 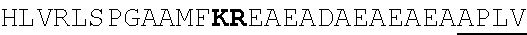 | |
| | | | RLSPGAAMFKRKAEADAEAEAPPLVRLSPG | |
| | | | 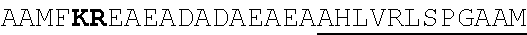 | |
| | | | 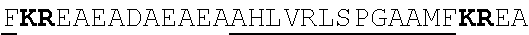 | |
| | | | EADADAEAEAAPLVRLSPGAAMFKREAEAD | |
| | | | 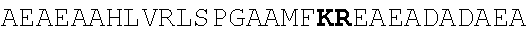 | |
| | | | EAAHLVRLSPGAAMFKREAEADADAEAGAD | |
| | | | ST | |
| 45 | Zr² | 1-- | MRLSIALGVTFGAVAGLTAPVEEVKRDADA | 116 |
| | | HFIELDPGQPMF | 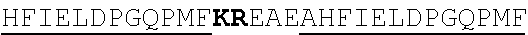 | |
| | | 2- | 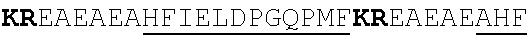 | |
| | | AHFIELDPGQPMF | 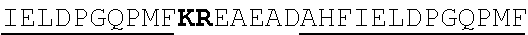 | |
| | | | 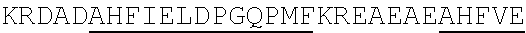 | |
| | | | 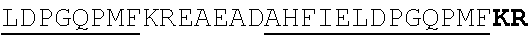 | |
| | | | GEIESAA | |

Green: Potential secretion signal sequences.
Bold: Potential Kex2 processing sites.
Orange: Potential Ste13 processing sites.
Underlined: Inferred mature peptide sequence.
For Species codes labeled with a reference, #1 peptide candidates have been postulated or tested before.

TABLE 9
Amino acid sequences of GPCRs.

| Code | Sequence | SEQ ID NO: |
|---|---|---|
| Sc | MSDAAPSLSNLFYDPTYNPGQSTINYTSIYGNGSTITFDELQGLVNST | 117 |
| | VTQAIMFGVRCGAAALTLIVMWMTSRSRKTPIFIINQVSLFLIILHSA | |
| | LYFKYLLSNYSSVTYALTGFPQFISRGDVHVYGATNIIQVLLVASIET | |
| | SLVFQIKVIFTGDNFKRIGLMLTSISFTLGIATVTMYFVSAVKGMIVT | |
| | YNDVSATQDKYFNASTILLASSINFMSFVLVVKLILAIRSRRFLGLKQ | |
| | FDSFHILLIMSCQSLLVPSIIFILAYSLKPNQGTDVLTTVATLLAVLS | |
| | LPLSSMWATAANNASKTNTITSDFTTSTDRFYPGTLSSFQTDSINNDA | |
| | KSSLRSRLYDLYPRRKETTSDKHSERTFVSETADDIEKNQFYQLPTPT | |
| | SSKNTRIGPFADASYKEGEVEPVDMYTPDTAADEEARKFWTEDNN | |
| | NL | |
| Scas1 | MSDAPPPLSELFYNSSYNPGLSIISYTSIYGNGTEVTFNELQSIVNKK | 118 |
| | ITEAIMFGVRCGAAILTIIVMWMISKKKKTPIFIINQVSLFLILLHSA | |
| | FNFRYLLSNYSSVTFALTGFPQFIHRNDVHVYAAASIFQVLLVASIEI | |
| | SLMFQIRVIFKGDNFKRIGTILTALSSSLGLATVAMYFVTAIKGIIAT | |
| | YKDVNDTQQKYFNVATILLASSINFMTLILVIKLILAIRSRRFLGLKQ | |
| | FDSFHILLIMSFQSLLAPSILFILAYSLDPNQGTDVLVTVATLLVVLS | |
| | LPLSSMWATAANNASRPSSVGSDWTPSNSDYYSNGPSSVKTESVKSDE | |
| | KVSLRSRIYNLYPKSKSEFEQSSEHTYVDKVDLENNFYELSTPITERS | |
| | PSSIIKKGKQGISTRETVKKLDSLDDIYTPNTAADEEARKFWSEDVSN | |
| | ELDSLQKIETETSDELSPEMLQLMIGQEEEDDNLLATKKITVKKQ | |
| Vp2 | MSGIDDMGDKPDILGLFYDANYDPGQGILTFISMYGNTTITFDELQLE | 119 |
| | VNSLITSGIMFGVRCGAACLTLLIMWMISKNKKTPIFIINQCSLILII | |
| | MHSGLYFKNILSNLNSLSYILTGFTQNITKNNIHVFGAANIIQVLLVA | |
| | TIELSLVFQIRVMFKGDSFRKAGYGLLSIASGLGIATVVMYFYSAITN | |
| | MIAVYNQTYNSTAKLFNVANILLSTSINFMTVVLIVKLFLAVRSRRYL | |
| | GLKQFDSFHILLIMSCQTLIVPSILFILSYALSTKLYTDHLVVIATLL | |
| | VVLSLPLSSMWASAANNSPKPSSFTTDYSNKNPSDTPSFYSQSISSSM | |
| | KSKFPSKFIPFNFKSKDNSSDTRSENTYIGNYDMEKNGSPNHSYSSKD | |
| | QSEVYTIGVSSMHTDIKSQKNISGQHLYTPSTEIDEEARDFWAGRAVN | |
| | NSVPNDYQPSELPASILEELNSLDENNEGFLETKRITFRKQ | |
| Vp1 | MSSQSHPPLIDLFYDSSYDPGESLIYYTSIYGNNTYITFDELQTIVNK | 120 |
| | KVTQGILFGVRCGAAFLMLVAMWLISKNKRSRIFITNQCCLVFMIMHS | |
| | GLYFRYLLSRYGSVTFILTGFQQLLTRNDIHIYGATDFIQVALVACIE | |
| | LSLIFQIKVIFAGTNYGKLANYFITLGSLLGLATFGMYMLTAINGTIK | |
| | LYNNEYDPNQRKYFNISTILLASSINMLTLILILKLVAAIRTRRYLGL | |
| | KQFDSFHILLIMSTQTLIIPSILFILSYSLREDMHTDQLIIIGNLIVV | |
| | LSLPLSSMWASSLNNSSKPTSLNTDFSGPKSSEEGTAISLLSQNMEPS | |
| | IVTKYTRRSPGLYPVSVGTPIEKEASYTLFEATDIDFESSSNDITRTS | |
| Td | MSDSAQNLSDLAFNSSYNPLDSFITFTSIYGDNTAVKFSVLQDMVDVN | 121 |
| | TNEAIVYGTRCGASVLTQIIMWMISKNRRTPVFIINQVSLTLILIHSA | |
| | LYFKYLLSGFGSVVYGLTAFPQLIKPGDLRAFAAANIVMVLLVASIEA | |
| | SLIFQVKVIFTGDNMKRVGLILTIICTCMGLATVTMYFITAVKSIVSL | |
| | YRDMSGSSTVLYNVSLIMLASSIHFMALILVVKLFLAVRSRRFLGLKQ | |
| | FDSFHILLIISCQTLLVPSLLFIIAYSFPSSKNIESLKAIAVLTVVLS | |
| | LPLSSMWATAANNFTNSSSSGSDSAPTNGGFYGRGSSNLYPEKTDNRS | |
| | PKGARNALYELRSKNNAEGQADIYTVTDIENDIFNDLSKPVEQNIFSD | |
| | VQIIDSHSLHKACSKEDPVMTLYTPNTAIEGEERKLWTSDCSCSTNGS | |
| | TPVKKKSTGEYANLPPHLLRYDENYDEEAGGRRKASLKW | |
| Sk | MSGKQDLSPLGLYSSYDPTKGLISYTSLYGSGTTVTFEELQIFVNKKI | 122 |
| | TQGILFGTRIGAAGLAIIVLWMVSKNRKTPIFIINQISLFLILLHSSL | |
| | FLRYLLGDYASVVFNFTLFSQSISRNDVHVYGATNMIQVLLVAAVEIS | |
| | LIFQVRVIFKGDSYKGVGRILTSISAVLGFTTVVMYFITAVKSMTSVY | |
| | SDLTKTSDRYFFNIASILLSSSVNFMTLLLTVKLILAVRSRRFLGLKQ | |
| | FDSFHVLLIMSFQTLIFPSILFILAYALNPNQGTDTLTSIATLLVTLS | |
| | LPLSSMWATSANNSSHPSSINTQFRQRNYDDVSFKTGITSFYSESSKP | |
| | SSKYRHTNNLYDLYPVSRTSNSRCNGYPNDGSKLAPNPNCVGHNGSTM | |
| | SVNDKNGAHATCVQNNVTLNTDSTLNYSNVDTQDTSKILMTT | |
| K1 | MSEEIPSLNPLFYNETYNPLQSVLTYSSIYGDGTEITFQQLQNLVHEN | 123 |
| | ITQATIFGTRIGAAGLALIIMWMVSKNRKTPIFIINQSSLVLTIVQSA | |
| | LYLSYLLSNFGGVPFALTLFPQMIGDRDKHLYGAVTLIQCLLVACIEV | |
| | SLVFQVRVIFKADRYRKIGIILTGVSASFGAATVAMWMITAIKSIIVV | |
| | YDSPLNKVDTYYYNIAVILLACSINFITLLLSVKLFLAFRARRHLGLK | |
| | QFDSFHILLIMSTQTLIGPSVLYILAYALNNKGVKSLTSIATLLVVLS | |
| | LPLTSIWAAAANDAPSASTFYRQFNPYSAQNRDDSSSYSYGKAFSDKY | |
| | SFSNSPQTSDGCSSKELELSTQLEMDLESGESFMDRAKRSDFVSSPGS | |
| | TDATVIKQLKASNIYTSETDADEEARAFWVNAIHENKDDGLMQSKTVF | |
| | KELR | |
| Zr | MSEINNSTYNPMNAYVTFTSIYGDDTMVRFKDVELVVNKRVTEAIMFG | 124 |
| | VKVGAASLTLIIMWMISKKRTTPIFIINQSSLVFTIIHASLYFGYLLS | |
| | GFGSIVYNMTSFPQLISSNDVRVYAATNIFEVLLVASIEISLVFQVKV | |
| | MFANNNGRRWTWCLMVVSIGMALATVGLYFATAVELIRAAYSNDTVSR | |
| | HVFYNVSLILLASSVNLMTLMLVVKLVLAIRSRRFLGLKQFDSFHILL | |
| | IMSCQTLIAPSILFILGWTLDPHTGNEVLITVGQLLIVLSLPLSSMWA | |
| | TTANNTSSSSSSVSCNDSSFGNDNLCSKSSQFRRTFMNRFRPKSVNGD | |
| | GNSENTFVTIDDLEKSVFQELSTPVSGESKIDHDHASSISCQKTCNHV | |
| | HASTVNSDKGSWSSDGSCGSSPLRKTSTVNSEDLPPHILSAYDDDRGI | |
| | VESKKIILKKL | |
| Zb | MSGLANNTSYNPLESFIIFTSVYGGDTMVKFEDLQLVFTKRITEGILF | 125 |
| | GVKVGAASLTMIVMWMISRRRTSPIFIMNQLSLVFTILHASFYFKYLL | |
| | DGFGSIVYTLTLFPQLITSSDLHVFATANVVEVLLVSSIEASLVFQVN | |
| | VMFAGSNHRKFAWLLVGFSLGLALATVALYFVTAVKMIASAYASQPPT | |
| | NPIYFNVSLFLLAASVFLMTLMLTVKLILAIRSRRFLGLKQFDSFHIL | |
| | LIMSCQTLIAPSVLYILGFILDHRKGNDYLITVAQLLVVLSLPLSSMW | |
| | ATTANDASSGTSMSSKESVYGSDSLYSKSKCSQFTRTFMNRFSTKPTK | |
| | NDEISDSAFVAVDSLEKNAPQGISEHVCEFPQSDLSDQATSISSRKKE | |
| | AVVYASTVDEDKGSFSSDINGYTVTNMPLASAASANCENSPCHVPRPY | |
| | EENEGVVETRKIILKKNVKW | |
| Cg | MEMGYDPRMYNPRNEYLNFTSVYDVNDTIRFSTLDAIVKGLLRIAIVH | 126 |
| | GVRLGAIFMTLIIMFISSNTWKKPIFIINMVSLMLVMIHSALSFHYLL | |
| | SNYSSISYILTGFPQLITSNNKRIQDAASIVQVLLVAAIEASLVFQIH | |
| | VMFTIENIKLIREIVLSISIAMGLATVATYLAAAIKLIRGLHDEVMPQ | |
| | THLIFNLSIILLASSINFMTFILVIKLFFAIRSRRYLGLRQFDAFHIL | |
| | LIMFCQSLLIPSVLYIIVYAVDSRSNQDYLIPIANLFVVLSLPLSSIW | |
| | ANTSNNSSRSPKYWKNSQTNKSNGSFVSSISVNSDSQNPLYKKIVRFT | |
| | SKGDTTRSIVSDSTLAEVGKYSMQDVSNSNFECRDLDFEKVKHTCENF | |
| | GRISETYSELSTLDTTALNETRLFWKQQSQCDK | |
| Ag | MGEEVSSFVEQYYDPNYDPSQSMLTYMSKFSNESTIKFEDLQEYINEN | 127 |
| | VMLGVFTGAKIAAAALALIILWMVTKRKRTPIYIVNQISLLLTVIHGI | |
| | LVLSGLLGGFSSSIFTLTLFPQCVNRSDIRLFVATNISMVSLIASIQV | |
| | SLVLQVHVIFRAGTHRRLGIFLTAVSAIIGFTTVCFYLVSAVLSVMAV | |
| | YQDIDNIGDTFFLSIAYICMAISVNFIFLLLSVKLLLAIRLRRFLGLK | |
| | QFDGLHILFIMSTQTIICPSILFILAFACEKNITDSLVYIAVLLVSLS | |
| | LPLSSVWATAANNATVPPFLNAHSLTSRYKAESWYTDSKNDAGSFSSS | |
| | ENCGSGYRHGRYSNNGGSSPHQCTGGDNTVIDIEKCQYRVNPTPHTSG | |
| | QFAFNQDSLETEFSEDTVVQIRTPNTEVEEEAKIFWARASITHENSSS | |
| | GVECGAHDMQTNVFKTPTSQTGSDCN | |
| Ss | MDTSINTLNPANIIVNYTLPNDPRVISVPFGAFDEYVNQSMQKAIIHG | 128 |
| | VSIGSCTIMLLIILIFNVKRKKSPAFYLNSVTLTAMIIRSALNLAYLL | |
| | GPLAGLSFTFSGLVTPETNFSVSEATNAFQVIVVALIEASMTFQVFVV | |
| | FQSPEVKKLGIALTSISAFTGAAAVGFTINSTIQQSRIYHSVVNGTPT | |
| | PTVATWSWVRDVPTILFSTSVNIMSFILILKLGFAIKTRRYLGLRQFG | |
| | SLHILLMMATQTLLAPSILILVHYGYGTSSNSQLILISYLLVVLSLPV | |
| | SSIWAATANNSPQLPSSATLSFMNKTTSHFSES | |
| Kp | MEEYSDSFDPSQQLLNFTSLYGETDATFAELDDYHFYVVKYAIVYGAR | 129 |
| | IGVGMFCTLMLFVVSKSWKTPIFVLNQSSLILLIIHSGFYIHYLTNQF | |
| | SSLTYMFTRIPNETHAGVDLRINVVTNTLYALLILSIEISLIYQVFVI | |
| | FKGVYENSLRWIVTIFTALFAAAVVAINFYVTTLQSVSMYNSNVDFPR | |
| | WASNVPLILFASSVNVVACLLLSLKLFFAIKVRRSLGLRQFDTFHILA | |
| | IMFSQTLIIPSILIVLGYTGTRDRDSLASLGFLLIVVSLPFSSMWAAT | |
| | ANNSNIPTSTGSFAWKNRYSPSTYSDDTTAVSKSFTIMTAKDECFTTD | |
| | TEGSPRFIKGDRTSEDLHF | |
| Cgu | MKSCSIGFGIPFINEPNFETVSILTMDVSFIDADVNPDNILLNFTIPG | 130 |
| | YQNGFSVPMVVINELQKSQMKYAIVYGCGVGASLILLFVVWILCSRKT | |
| | PLFIMNNIPLVLYVISSSLNLAYITGPLSSVSVFLTGILTSHDAINVV | |
| | YASNALQMLLIFSIQSTMAYHVYVMFKSPQIKYLRYMLVGFLGCLQIV | |
| | TTCLYINYNVLYSRRMHKLYETGQTYQDGTVMTFVPFILFQCSVNFSS | |
| | IFLVLKLIMAIRTRRYLGLRQFGGFHILMIVSLQTMLVPSILVLVNYA | |
| | AHKAVPSNLLSSVSMMIIVLSLPASSMWAAAANASSAPSSAASSLFRY | |
| | TTSDSDRTLETKSDHFIMKHESHNSSPNSSPLTLVQKRISDATLELPK | |
| | ELEDLIDSTSI | |
| Cp | MNKIVSKLSSSDVIVTVTIPNEEDGTYEVPFYAIDNYHYSRMENAVVL | 131 |
| | GATIGACSMLLIMLIGILFKNFQRLRKSLLFNINFAILLMLILRSACY | |
| | INYLMNNLSSISFFFTGIFDDESFMSSDAANAFKVILVALIEVSLTYQ | |
| | IYVMFKTPMLKSWGIFASVLAGVLGLATLATQIYTTVMSHVNFVNGTT | |
| | GSPSQVTSAWMDMPTILFSVSINVLSMFLVCKLGLAIRTRRYLGLKQF | |
| | DAFHILFIMSTQTMIIPSIILFVHYFDQNDSQTTLVNISLLLVVISLP | |
| | LSSLWAQTANNVRRIDTSPSMSFISREASNRSGNETLHSGATISKYNT | |
| | SNTVNTTPGTSKDDSLFILDRSIPEQRIVDTGLPKDLEKFINNDFYED | |
| | DGGMIAREVTMLKTAHNNQ | |
| Cau | MEFTGDIVLKYTLGGEEYLSTFEQLDSSVNRSLELGVVHGIAIACGVL | 132 |
| | LMVLAWVIIIKKKNPIFVLNQLTLLLMVIKSSLYLAFLFGPLSSLTYK | |
| | FTRVLPHDKWHAFHVYIATNVIHTLLIATVEMTLVFQIYIIFKSPEVR | |
| | HLGYILTGAASALALTIVALYIHSTVISAVQLKEQLLMHEIKITNSWV | |
| | NNVPIILFSASLNVVCIILIAKLALAIKTRRYLGLKQFDGLHILMITS | |
| | TQTFIVPSVLMIVNYKQSSSYLTLLANISVILVVCNLPLSSLWAASAN | |
| | NSSTPTSSANTVFSRWDSKFSDTETIAHELPLIPGKAEKLQLVSPITE | |
| | KGDTHTMCESHGDQDLIDKMLDDIEGAVMTTEFNLNNRTV | |
| Y1 | MQLPPRPDFDIATLVASITVPETELVLGQMPLGALEQLYQNRLRLAIL | 133 |
| | FGVRVGAAVLTLIAMHLISKKNRTKILFLANQMSLIMLIIHAALYFRF | |
| | LLGPFASMLMMVAYIVDPRSNVSNDISVSVATNVFMMLMIMSVQLSLA | |
| | VQTRSVFHAWLKSRIYVTVGLILLSLVVFVFWTTHTIVSCIVLTHPTR | |
| | DLPSMGWTRLASDVSFACSISFASLVLLAKLVTAIRVRKTLGKKPLGY | |
| | TKVLVIMSTQSLVVPSILIIVNYALPEKNSWILSGVAYLMVVLSLPLS | |
| | SIWATAVHDDEMQSNYLLSALKDGHVQPSESKLKTVFLNRLRPFSTTT | |
| | NRDDESSVDSPAMPSPESDVTFLNTGFECDEKM | |
| C1 | MNPADINIEYTLGDTAFSSTFADFEAWKTRNTQFAIVNGVALACGIIL | 134 |
| | MVVSWIIIVNKRAPIFAMNQTMLVIMVIKSAMYLKHIMGPLNSLTFRF | |
| | TGLMEESWAPYNVYVTINVLHVLLVAAVESSLVFQIHVVFKSSRARVA | |
| | GRAIVSAMSTLALLIVSLYLYSTVRHAQTLRAELSHGDTTTVEPWVDN | |
| | VPLILFSASLNVLCLLLALKLVFAVRTRRHLGLRQFDSFHILIIMATQ | |
| | TFVIPSSLVIANYRYASSPLLSSISIIVAVCNLPLCSLWACSNNNSSY | |
| | PTSSQNTILSRYETETSQATDASSTTCAGIAEKGFDKSPDSPTFGDQD | |
| | SVSISHILDSLEKDVEGVTTHRLT | |
| Ca | MNINSTFIPDKPGDIIISYSIPGLDQPIQIPFHSLDSFQTDQAKIALV | 135 |
| | MGITIGSCSMTLIFLISIMYKTNKLTNLKLKLKLKYILQWINQKIFTK | |
| | KRNDNKQQQQQQQQQIESSSYNNTTTTTSGSYKLFLFYLNSLILLIGI | |
| | IRSGCYLNYNLGPLNSLSFVFTGWYDGSSFISSDVTNGFKCILYALVE | |
| | ISLGFQVYVMFKTSNLKIWGIMASLLSIGLGLIVVAFQINLTILSHIR | |
| | FSRAISTNRSEEESSSSLSSDSVGYVINSIWMDLPTILFSISINIMTI | |
| | LLIGKLIIAIRTRRYLGLKQFDSFHILLIGFSQTLIIPSIILVVHYFY | |
| | LSQNKDSLLQQISLLLIILMLPLSSLWAQTANNTHNINSSPSLSFISR | |
| | HHSSDSSRSGGSNTIVSNGGSNGGGGGGGNFPVSGIDAQLPPDIEKIL | |
| | HEDNNYKLLNSNNESVNDGDIIINDEGMITKQITIKRV | |
| Ct | MDINNTIQSSGDIIITYTIPGIEEPFELPFEVLNHFQSEQSKNCLVMG | 136 |
| | VMIGSCSVLLIFLVGILFKTNKFSTIGKSKNLSKNFLFYLNCLITFIG | |
| | IIRAACFSNYLLGPLNSASFAFTGWYNGESYASSEAANGFRVILFALI | |
| | ETSMVFQVFVMFRGAGMKKLAYSVTILCTALALVVVGFQINSAVLSHR | |
| | RFVNTVNEIGDTGLSSIWLDLPTILFSVSVNLMSVLLIGKLIMAIKTR | |
| | RYLGLKQFDSFHVLLICSTQTLLVPSLILFVHYFLFFRNANVMLINIS | |
| | ILLIVLMLPFSSLWAQTANTTQYINSSPSFSFISREPSANSTLHSSSG | |
| | HYSEKSYGINKLNTQGSSPATLKDDHNSVILEATNPMSGFDAQLPPDI | |
| | ARFLQDDIRIEPSSTQDFVSTEVTYKKV | |
| Cn | MDSYLLNHPGDISLNFALPLSDEVYTITFNDLDSQSSFSIQYLVIHSC | 137 |
| | AITVCLTLLVLLNLFIRNKKTPVFVLNQVILFFAIVRSSLFIGFMKSP | |
| | LSTITASFTGIISDDQKHFYKVSVAANAALIILVMLIQVSFTYQIYHF | |
| | RSPEVRKFGVFMTSALGVLMAVTFGFYVNSAVASTKQYQHIFYSTDPY | |
| | IMDSWVTGLPPILYSASVIAMSLVLVLKLVAAVRTRRYLGLKQFSSYH | |
| | ILLIMFTQTLFVPTILTILAYAFYGYNDILIHISTTITVVLLPFTSIW | |
| | ASIANNSRSLMSAASLYFSGSNSSLSELSSPSPSDNDTLNENVFAFFP | |
| | DKLQKMNSSEAVSAVDKVVVHDHFDTISQKSIPHDILEILQGNEGGQM | |
| | KEHISVYSDDSFSKTTPPIVGGNLLITNTDIGMK | |
| Le | MDEAINANLVSGDIIVSFNIPGLPEPVQVPFSEFDSFHKDQLIGVIIL | 138 |
| | GVTIGACSLLLILLLGMLYKSREKYWKSLLFMLNVCILAATILRSGCF | |
| | LDYYLSDLASISYTFTGVYNGTSFASSDAANVFKTIMFALIETSLTFQ | |
| | VYVMFQGTTWKNVVGHAVTALSGLLSVASVAFQIYTTILSHNNFNATI | |
| | SGTGTLTSGVWMDLPTLLFAASINFMTILLLFKLGMAIRQRRYLGLKQ | |
| | FDGFHILFIMFTQTLFIPSILLVIHYFYQAMSGPFIINMALFLVVAFL | |
| | PLSSLWAQTANTTKKIESSPSMSFITRRKSEDESPLAANDEDRLRKFT | |
| | TTLDLSGNKNNTTNNSNMNNMSNINYPSTGLGEDDKSFIFEMEPSRER | |
| | AAIEEIDLGARIDTGLPRDLEKFLVDGFDDSDDGEGMIAREVTMLKK | |
| Gc | MAEDSIFPNNSTSPLTNPIVVETIKGTAYIPLHYLDDLQYEKMLLASL | 139 |
| | FSVRIATSFVVIIWYFVAVNKAKRSKFLYIVNQVSLLIVFIQSILSLI | |
| | YVFSNFSKMSTILTGDYTGITKRDINVSCVASVFQFLFIACIELALFI | |
| | QATVVFQKSVRWLKFSVSLIQGSVALTTTALYMAIIVQSIYATLNPYA | |
| | GNLIKGRFGYLLASLGKIFFSISVTSCMCIFVGKLVFAIHQRRTLGIK | |
| | QFDGLQILVIMSTQSMIIPTIIVLMSFLRRNAGSVYTMATLLVALSLP | |
| | LSSLWAEAKTTRDSASYTAYRPSGSPNNRSLFAIFSDRLACGSGRNNR | |
| | HDDDSRGNGSVNARKADVESTIEMSSCYTDSPTYSKFEAGLDARGIVF | |
| | YNEHGLPVVSGEVGGSSSNGTKLGSGHKYEVNTTVVLSDVDSPSPTDV | |
| | TRK | |
| Bm | MASNGWQNNATFDPYAQTFVLLQPDGLTPFPALLGDVLALNTVSVTQG | 140 |
| | HYGTQVGISGLLLLILLIMTKPDKRRSLVFILNSLSLLLIFARNVLSC | |
| | VQLTTIFYNFYNWELHWYPESPALSRAMDLSAATEVLNIPIDVAIFSS | |
| | LVVQVHIVCCTIHTLVRTSALLSSAAVGLAAVAVRFALAVVNIKYSIF | |
| | GINTLTEPQFNLIVHLKRVSDILTVVAIAFFSSIFVAKLGVAIHTRRT | |
| | LNLKNFGAIQIIFIMGCQTMLIPLIFVIVSFYASRGSQIGSMVPTVVA | |
| | TFLPLSGMWASAQTNNEKMGRADQRFHRAVPVGATDFSVTKARSAKAS | |
| | DTLDTLIGDD | |
| So | MREPWWKNYYTMNGTQVQNQSIPILSTQGYIQVPLSTIDKAERNRILT | 141 |
| | GMTVSAQLALGVLIMVMSILLSSPEKRKTPVFIVNSASIISMCIRAIL | |
| | MIVNLCSESYSLAVMYGFVFELVGQYVHVFDILVMIIGTIIIITAEVS | |
| | MLLQVRIICAHDRKTQRIVTCISSGLSLIVVAFWFTDMCQEIKYLLWL | |
| | TPYNNHQISGYYWVYFVGKILFAVSIMFHSAVFSYKLFHAIQIRKKIG | |
| | QFPFGPMQCILIISCQCLFVPAIFTIIDSFIHTYDGFSSMTQCLLIVS | |
| | LPLSSLWASSTALKLQSLKSTTSPGDTTQVSIRVDRTYDIKRIPTEEL | |
| | SSVDETEIKKWP | |
| Tm | MEQIPVYERPGFNPHKQNITLFKHDGSTVTVGLHELDAMFTHSIRVAV | 142 |
| | VFASQIGACALLSVIVAMVTKREKRRALFFLHIISLLLVVVRSVLQIL | |
| | YFVGPWAETYNYVAYYYEDIPLSDKLISIWAGIIQLILNICILLSLIL | |
| | QVRVVYATSPKLNTIMTLVSCVIASISVGFFFTVIVQISEAILNGVGY | |
| | DGWVYKVHRGVFAGAIAFFSFIFIFKLAFAIRRRKALGLQRFGPLQVI | |
| | FIMGCQTMIVPAIFATLENGVGFEGMSSLTATLAVISLPLSSMWAAAQ | |
| | TDGPSPQSTPRDGYRRFSTRRSALNRSDPSGGRSVDMNTLDSTGNDSL | |
| | ALHVDKTFTVESSPSSQSQAGPHKERGFEFA | |
| Ao | MDSKFDPYSQNLTFHAADGTPFQVPVMTLNDFYQYCIQICINYGAQFG | 143 |
| | ASVIIFIILLLLTRPDKRASSVFFLNGGALLLNMGRLLCHMIYFTTDF | |
| | VKAYQYFSSDYSRAPTSAYANSILGVVLTTLLLVCIETSLVLQVQVVC | |
| | ANLRRRYRTVLLCVSILVALIPVGLRLGYMVENCKTIVQTDTPLSLVW | |
| | LESATNIVITISICFFCSIFIIKLGFAIHQRRRLGVRDFGPMKVIFVM | |
| | GCQTLTVPALLSILQYAVSVPELNSNIMTLVTISLPLSSIWAGVSLTR | |
| | SSSTENSPSRGALWNRLTDSTGTRSNQTSSTDTAVAMTYPSNKSSTVC | |
| | YADQSSVKRQYDPEQGHGISVEHDVSVHSCQRL | |
| Sp | MRQPWWKDFTIPDASAIIHQNITIVSIVGEIEVPVSTIDAYERDRLLT | 144 |
| | GMTLSAQLALGVLTILMVCLLSSSEKRKHPVFVFNSASIVAMCLRAIL | |
| | NIVTICSNSYSILVNYGFILNMVHMYVHVFNILILLLAPVIIFTAEMS | |
| | MMIQVRIICAHDRKTQRIMTVISACLTVLVLAFWITNMCQQIQYLLWL | |
| | TPLSSKTIVGYSWPYFIAKILFAFSIIFHSGVFSYKLFRAILIRKKIG | |
| | QFPFGPMQCILVISCQCLIVPATFTIIDSFIHTYDGFSSMTQCLLIIS | |
| | LPLSSLWASSTALKLQSMKTSSAQGETTEVSIRVDRTFDIKHTPSDDY | |
| | SISDESETKKWT | |
| Af | MNSTFDPWTQNITLTQSDGTTVISSLALADDYLHYMIRLGINYGAQLG | 145 |
| | ACAVLLLVLLLLTRPEKRVSSVFVLNVAALLANIIRLGCQLSYFSTGF | |
| | ARMYALLAGDFSRVSRGAYAGQVMASVFFTIVFICVEASLVLQVQVVC | |
| | SNLRRQYRILLLGASTLAALVPIGVRLTYSVLNCMVIMHAGTMDHLDW | |
| | LESATNIVTTVSICFFCAVFVVKLGLAIKMRKRLGVKQFGPMRVIFIM | |
| | GCQTMTIPAIFAICQYFSRIPEFSHNVLTLVIISLPLSSIWAGFALVQ | |
| | ANSTARSTESRHHLWNILSSDGATRDKPSQCVSSPMTSPTTTCYSEQS | |
| | TSKPQQDPENGFGISVAHDISIHSFRKDAHGDI | |
| Pd | MSTANVHLPADFDPTRQNITIYTPDGTPVVATLPMINLFNRQNNEICV | 146 |
| | VYGCQLGASLIMFLVVLLTTRVSKRKSPIFVLNVLSLIISCLRSLLQI | |
| | LYYIGPWTEIYRYLSFDYSTVPASAYANSVAATLLTLFLLITIEASLV | |
| | LQTNVVCKSMSSHIRWPVTALSMVVSLLAISFRFGLTIRNIEGILGAT | |
| | VKSDSLMFSGASLISETASIWFFCTIFVIKLGWTLYQRKKMGLKQWGP | |
| | MQIITIMAGCTMLIPSLFTVLEFFPEETFYEAGTLAICLVAILLPLSS | |
| | VWAAAAIDGDEPVRPHGSTPKFASFNMGSDYKSSSAHLPRSIRKASVP | |
| | AEHLSRTSEEELGDDGTLNRGGAYGMDRMSGSISPRGVRIERTYEVHT | |
| | AGRGGSIEREDIF | |
| Sj | MYSWDEFRSPKQAEVLNQTVTLETIVSTIQLPISEIDSMERNRLLTGM | 147 |
| | TVAVQVGLGSFILVLMCIFSSSEKRKKPVFIFNFAGNLVMTLRAIFEV | |
| | IVLASNNYSIAVQYGFAFAAVRQYVHAFNIIILLLGPFILFIAEMSLM | |
| | LQVRIICSQHRPTMITTTVISCIFTVVTLAFWITDMSQEIAYQLFLKN | |
| | YNMKQIVGYSWLYFIAKITFAASIIFHSSVFSFKLMRAIYIRRKIGQF | |
| | PFGPMQCIFIVSCQCLIVPAIFTLIDSFTHTYDGFSSMTQCLLIISLP | |
| | LSSLWATHTAQKLQTMKDNTNPPSGTQLTIRVDRTFDMKFVSDSSDGS | |
| | FTEKTEETLP | |
| Pb | MAPSFDPFNQNVVFHKADGTPFNVSIHELDDFVQYNTRVCINYSSQLG | 148 |
| | ASVIAGLMLAMLTHSEKRRLPVFFLNTFALAMNFARLLCMTIYFTTGF | |
| | NKSYAYFGQDYSQVPGSAYAASVLGVVFTTLLVISMEMSLLIQTRVVC | |
| | TTLPDIQRYLLMAVSSAISLMAIGFRLGLMVENCIAIVQASNFAPFIW | |
| | LQSASNITITISTCFFSAVFVTKLAYALVTRIRLGLTRFGAMQVMFIM | |
| | SCQTMVIPAIFSILQYPLPKYEMNSNLFTLVAIFLPLSSLWASVATKS | |
| | SFETSSSGRHQYLWPSEQSNNVTNSEIKYQVSFSQNHTTLRSGGSVAT | |
| | TLSPDRLDPVYSEVEAGTKA | |
| Mg | MVVTAPPSVDRTYFIPNSTFDPYQQDLTLVYPDGVHALVANVDDIVYF | 149 |
| | MGLAVKSTLIFAIQIGISFVLMLVIALLTKPERRVTLVFFLNMTALFT | |
| | IFIRAILMCTTFVGTYYNFYNWIMGNYPNSGLADRVSIAAEVFAFLII | |
| | LSLELSMMFQVRIVCINLSSFRRRIITFSSIVVAMIVCTVRFALMVLS | |
| | CDWRIVNIGDATQEKNRIINRVASGYNICTIASIIFFNTIFVSKLAVA | |
| | IKHRRSMGMKQFGPMQIIFVMGCQTLLIPAIFGIISYFALASTQVYSL | |
| | MPMVVAIFLPLSSMWASFNTNKTNSVTNMRQPNVYRPNMIIGQDTTQN | |
| | SGKNTNISGTSNSTATTSSFASDKRRLNLSFNTQGTLVNSISEEEVNN | |
| | PQKLGPSATVAVMDRDSLELEMRQHGIAQGRSYSVRSD | |
| Pr | MATSSPIQPFDPFTQNVTFRLQDGTEFPVSVKALDVFVMYNVRVCINY | 150 |
| | GCQFGASFVLLVILVLLTQSDKRRSAVFILNGLALFLNSSRLLFQVIH | |
| | FSTAFEQVYPYVSGDYSSVPWSAYAISIVAVVLTTLVVVCIEASLVIQ | |
| | VHVVCSTLRRRYRHPLLAISILVALVPIGFRCAWMVANCKAIIKLTYT | |
| | NDVWWIESATNICVTISICFFCVIFVTKLGFAIKQRRRLGVREFGPMK | |
| | VIFVMGCQTMVVPAIFSITQYYVVVPEFSSNVVTLVVISLPLSSIWAG | |
| | AVLENARRTGSQDRQRRRNLWRALVGGAESLLSPTKDSPTSLSAMTAA | |
| | QTLCYSDHTMSKGSPTSRDTDAFYGISVEHDISINRVQRNNSIV | |
| An | MATHNQISDQCQWSYPEVFTTQAVEEPTAEPASYHLHSTLTIMASNFD | 151 |
| | PWNQTITFRLEDGTPFDISVDYLDGILQYSIRACVNYAAQLGASVILF | |
| | VILVLLTRAEKRASCLFWLNSLALLLNFARLLCDVLFFTGNFVRIYTL | |
| | ISADESRVTASDLATSIVGAIMTALLLTTIEISLVLQVQVVCSNLRRI | |
| | YRRALLCVSAVVATATIAIRYSLLAVNIRAILEFSDPTTYNWLESLAT | |
| | VALTISICYFCVIFVTKLGFAIRLRRKLGLSELGPMKVVFIMGCQTLV | |
| | IPGKRTLSSLIPPVIVSITHYVSDVPELQTNVLTIVALSLPLSSIWAG | |
| | TTIDKPVTHSNVRNLWQILSFSGYRPKQSTYIATTTTATTNAKQCTHC | |
| | YSESRLLTEKESGRNNDTSSKSSSQYGIAVEHDISVRSARRESFDV | |
| Sn | MASMVPPPDFDPYTQEFMVLGPDGQEIPISMQTVNEYRLYTARLGLAY | 152 |
| | GSQIGATLLLLLVLSLLTRREKRKSGIFIVNALCLVTNTIRCILLSCF | |
| | VTSTLWHPYTQFSQDTSRVSKTDVNTSIAASIFTLIVTVLIMISLSVQ | |
| | VWVVCITTAPYQRYMIMGATTATAMVAVGYKAAFVITSIIQTLNGQDG | |
| | GSYLDLVMQSYITQAVAISFYSCIFTYKLGHAIVQRRTLNMPQFGPMQ | |
| | IIFIMGSLFTGLQFVKNVDELGIITPTIVCIFLPLSAIWAGVVNEKVV | |
| | GANGPDAHHRLLQGEFYRAASNSTYGSNSSGTVVDRSRQMSVCTCASS | |
| | SPFVRKKSVAEWDDEAILVGREFGFSRGEVGERG | |
| Hj | MSSFDPYTQNITILVSPSSPPISIPIPVIDAFNDETASIITNYAAQLG | 153 |
| | AALAMLLVLLAATPTARLLRADGPSLLHALALLVCVVRTVLLIYFFLT | |
| | PFSHFYQVWTGDFSQVPAWNYRASIAGTVLSTLLTVVTDAALVNQAWT | |
| | MVSLFAPRTKRAVCVLSLLITLLAISFRVAYTVIQCEGIAELAAPRQY | |
| | AWLIRATLIFNICSIAWFCALFNSKLVAHLVTNRGVLPSRRAMSPMEV | |
| | LIMANGILMIVPVVFAILEWHHFINFEAGSLTPTSIAIILPLSSLAAQ | |
| | RIANTSSS | |
| Bc | MASNSSNFDPLTQSITILMADGITTVSFTPLDIDFFYYYNVACCINYG | 154 |
| | AQAGACLLMFFVVVVLTKAVKRKTLLFVLNVLSLIFGFLRAMLYAIYF | |
| | LQGFNDFYAAFTFDFSRVPRSSYASSVAGSVIPLCMTITVNMSLYLQA | |
| | YTVCKNLDDIKRIILTTLSAIVALLAIGFRFAATVVNSVAILATSASS | |
| | VPMQWLVKGTLVTETISIWFFSLIFTGKLVWTLYNRRRNGWRQWSAVR | |
| | ILAAMGGCTMVIPSIFAILEYVTPVSFPEAGSIALTSVALLLPISSLW | |
| | AGMVTDEETSAIDVSNLTGSRTMLGSQSGNFSRKTHASDITAQSSHLD | |
| | FSSRKGSNATMMRKGSNAMDQVTTIDCVVEDNQANRGLRDSTEMDLEA | |
| | MGVRVNKSYGVQKA | |
| Bb | MDGSSAPSSPTPDPTFDRFAGNVTFFLADHITTTSVPMPVLNAYYDES | 155 |
| | LCTTMNYGAQLGACLVMLVVVVALTPAAKLARRPASALHLVGLLLCAV | |
| | RSGLLFAYFVSPISHFYQVWAGDFSAVSRRYWDASLAANTLAFPLVVV | |
| | VEAALINQAWTMVAFWPRAAKAAACACSAVIVLLTIGTRLAYTIVQNH | |
| | AIVTAVPPEHFLWAIQWSAVMGAVSIFWFCAVFNVKLVCHLVANRGIL | |
| | PSISVVNPMEVLVMTNGTLMIIPSIFAGLEWAKFTNFESGSLTLTSVI | |
| | IILPLGTLAAQRISGQGSQGYQAGHLFHEQQQQQARTRSGAFGSASQQ | |
| | SHPTNKVPSSITLSTSGTPITPQISAGSRPELPLVDRSERLDPIDLEL | |
| | GRIDAFRGSSDFSPSTARPKRMQRDNFA | |
| Nc | MASSSSPPADIFSGITQSLNSTHATLTLPIPPADRDHLENQVLFLFDN | 156 |
| | HGQLLNVTTTYIDAFNNMLVSTTINYATQIGATFIMLAIMLLMTPRRR | |
| | FKRLPTIISLLALCINLIRVVLLALFFPSHWTDFYVLYSGDWQFVPPG | |
| | DMQISVAATVLSIPVTALLLSALMVQAWSMMQLWTPLWRALVVLVSGL | |
| | LSLVTVAMSFANCIFQAKNILYADPLPSYVVVRKLYLALTTGSISWFT | |
| | FLFMIRLVMHMWTNRSILPSMKGLKAMDVLIITNSILMLIPVLFAGLE | |
| | FLDSASGFESGSLTQTSVVIVLPLGTLVAQRIATRGYMPDSLEASSGP | |
| | NGSLPLSNLSFAGGGGGGSGGHKDKENGGGIIPPTTNNTAATNFSSSI | |
| | ACSGISCLPKVKRMTASSASSSQRPLLTMTNSTIASNDSSGFPSPGIH | |
| | NTTTTTTQYQYSMGMNMPNFPPVPFPGYQSRTTGVTSHIVSDGRHHQG | |
| | MNRHPSVDHFDRELARIDDEDDDGYPFASSEKAVMHGDDDDDVERGRR | |
| | RALPPSLGGVRVERTIETRSEERMPSPDPLGVTKPRSFE | |
| She | MKPAAGPASSPFDPFNQTFYLTGPDNTTVPVSVPQVDYIWHYIIGTSI | 157 |
| | NYGSQIGACLLMLLVMLTLTSKSRFSRAATLINVASLLIGVIRCVLLA | |
| | VYFTSSLTELYALFVGDYSQVRRSDLCVSAVATFFSLPQLVLIEAALF | |
| | LQAYSMIKMWPSLWRAVVLAMSVVVAVCAIGFKFASVVMRMRSTLTLD | |
| | DSLDFWLVEVDLAFTATTIFWFCFIYIIRLVIHMWEYRSILPPMGSVS | |
| | AMEVLVMTNGALMLVPVIFAAIEINGLSSFESGSLVHTSVIVLLPLGS | |
| | LIAQAMTRPDGYVQRTNTSGASGASGAHPGRNGSGHGGHGGAYSRAMT | |
| | NTLNTLDTLDTVDSKTSIMHHHHHHHRNHSNGMSKTKANSGTWSHASD | |
| | ANSTNAMISGGIATQVRIQANQSTLGNTGMSGGSGAPNSHTRNNSLAA | |
| | MEPVEKQLHDIDATPLSASDCRVWVDREVEVRRDMV | |
| Mo | MDQTLSATGTATSPPGPALTVDPRFQTITMLTPALMGQGFEEVQTTPA | 158 |
| | EINDVYFLAFNTAIGYSTQIGACFIMLLVLLTMTAKARFARIPTIINT | |
| | AALVVSIIRCTLLVIFFTSTMMEFYTIFSDDFSFVHPNDIRRSVAATV | |
| | FAPLQLALVEAALMVQAWAMVELWPRAWKVSGIAFSLILATVTVAFKC | |
| | ASAAVTVKSALEPLDPRPYLWIRQTDLAFTTAMVTWFCFLFNVRLIMH | |
| | MWQNRSILPTVKGLSPMEVLVMANGLLMVFPVLFAGLYYGNFGQFESA | |
| | SLTITSVVLVLPLGTLVAQRLAVNNTVAGSSANTDMDDKLAFLGNATT | |
| | VTSSAAGFAGSSASATRSRLASPRQNSQLSTSVSAGKPRADPIDLELQ | |
| | RIDDEDDDFSRSGSAGGVRVERSIERREERL | |
| Dh | MDHNTQHFNRPEYIEIPVPPSKGFNPHTNPAFFIYPDGSNMTFWFGQI | 159 |
| | DDFRRDQLFTNTIFSIQIGAALVILCVMFCVTHADKRKTIVYLLNVSN | |
| | LFVVIIRGVFFVHYFMGGLARTYTTFTWDTSDVQQSEKATSIVSSICS | |
| | LILMIGTQISLLLQVRICYALNPRSKTAILVTCGSISGIATTAYLLLG | |
| | AYTIQLREKPPDMKFMKWAKPVVNALVALSIVSFSGIFSWRMFQSVRN | |
| | RRRMGFTGIGSLESLLASGFQCLVFPGLVTTALTVAGSTWYIAVNLTT | |
| | PSDLTAIYNCSAFFAYAFSIPLLKERAQVEKTISVVIAIAGVLVVAYG | |
| | DGADDGSTSNGEKARLGGNVLIGIGSVLYGLYEVLYKKLLCPPSGASP | |
| | GRSVVFSNTVCACIGAFTLLFLWIPLPLLHWSGWEIFELPTGKTAKLL | |
| | GISIAANATFSGSFLILISLTGPVLSSVAALLTIFLVAITDRILFGRE | |
| | LTSAAILGGLLIIAAFALLSWATWKEMIEENEKDTIDSISDVGDHDD | |
| Fg | MSKEAFDPFTQNVTFFAPDGKTEINIPVAAIDQVRRMMVNTTINYATQ | 160 |
| | LGACLIMLVVILVMVPKEKFRRPFMILQIASLVICCCRMLLLSIFHSS | |
| | QFLDFYVFWGDDHSRIPRSAYAPSVAGNTMSLCLVISVETMLMSQAWT | |
| | MVRLWPNVWKYIIAGISLVVSIVAISVRLAYTIIQNNAVLKLEPAFHM | |
| | FWLIKWTVIMNVASISWWCAIFNIKLVWHLISNRGILPSYKTFTPMEV | |
| | LIMTNGILMIIPVIFASLEWAHFVDFESASLTLTSVAVILPLGTLAAQ | |
| | RIASSAPNSANSTGASSGIRYGVSGPSSFTGFKAPSFSTGTTDRPHVS | |
| | IYARCEAGTSSREHINPQDVELAKLDPETDHHVRVDRAFLQREERIRA | |
| | PL | |
| Cc | MAARIIPALTLTAPTSYPTAGVGGYYYDTAFGVPTYSSAAFNQTTWRL | 161 |
| | LDNWDHINVNYASSEGLAAGLGWATLIYLLALTPSHKRTTPFHCFLLV | |
| | GLIFLLGHLMVNIIAALTPGLNTTSAYTYVTLDTSSSVWPRKYIAVYA | |
| | VNAVASWFAFIFATICLWLQAKGLMTGIRVRFIIVYKIILMYLIVAAV | |
| | IALAICMAFNIQQILYIGKPVELADGTALLRLRNAYLITYAISIGSFS | |
| | LVSICSIMDIIWRRPSRVIKGHNIFASALNLVGLLCAQSFVVPCEYKR | |
| | ALGQVPDCTTFADHIFHTVIFCILQVIPNSSGVMLPEIMLLPSVYVIL | |
| | PLGSLFMTVNSPESDVNKTSFPPKSSPGPFDRSPTLTSGTLPGSRPES | |
| | YVLDMASDKNSGNRKSVCSQFDRELNLIDSLDTLSGREGDSMLHAQSN | |
| | NNNQTREQDKQPRADTTHVGSENMV | |

Inference of the amino acid sequences of peptide ligands. The amino acid sequences of the mature peptide ligands were either taken from literature (Table 4) or predicted using the method reported by Martin et al⁶⁶. In brief, mating pheromone precursor genes have a relatively conserved architecture. Genes encode for an N-terminal secretion signal (pre-sequence at the amino acid level), followed by repetitive sequences of the pro-peptide composed of non-homologous pro sequences, homologous sequences belonging to the presumptive signal peptide and protease processing sites. Based on this conserved arrangement, the actual sequence of the secreted peptide ligand can be predicted from the precursor sequence. Alignment with reported functional pheromone precursor sequences (from S. cerevisiae and C. albicans) facilitated annotation.

Construction of GPCR expression vectors. The GPCR expression vector is based on pRS416 (URA3 selection marker, CEN6/ARS4 origin of replication). All GPCRs were cloned under control of the constitutive S. cerevisiae TDH3 promoter and terminated by the S. cerevisiae STE2 terminator. Unique restriction sites (SpeI and XhoI) flanking the GPCR coding sequence were used to swap GPCR genes. Most GPCRs were codon-optimized for S. cerevisiae, DNA sequences were ordered as gBlocks, amplified with primers giving suitable homology overhangs and inserted into the linearized acceptor vector by Gibson Assembly. DNA sequences of all GPCR genes as well as the sequence of the full expression cassette (GPDp-xy.Ste2-Ste2t) integrated into the ΔSte2 locus are listed in Table 5.

TABLE 5
Sequences of codon-optimized GPCR genes, expression cassette and genomic
integration design (STE2 locus and STE3 locus). Codon-optimized GPCR genes were
cloned into vector pRS416 under control of the constitutive TDH3 promoter
and the Ste2 terminator. The first row shows the sequence of the generic
GPCR expression cassette. The second row shows the STE2 locus replaced by the
generic expression cassette. Codon-optimized sequences of the indicated
GPCRs have been reported previously in Ostrov, N. et al. A modular yeast
biosensor for low-cost point-of-care pathogen detection. Science advances 3,
e1603221 (2017), and are indicated in Table 5 by a superscript ‘10’.
TDH3p-xy.Ste2-Ste2t expression cassette
AGTTTATCATTATCAATACTGCCATTTCAAAGAATACGTAAATAATTAATAGTAGTGATTTTCCTAACTTTATTTAGTCA
AAAAATTAGCCTTTTAATTCTGCTGTAACCCGTACATGCCCAAAATAGGGGGCGGGTTACACAGAATATATAACATCGTA
GGTGTCTGGGTGAACAGTTTATTCCTGGCATCCACTAAATATAATGGAGCCCGCTTTTTAAGCTGGCATCCAGAAAAAAA
AAGAATCCCAGCACCAAAATATTGTTTTCTTCACCAACCATCAGTTCATAGGTCCATTCTCTTAGCGCAACTACAGAGAA
CAGGGGCACAAACAGGCAAAAAACGGGCACAACCTCAATGGAGTGATGCAACCTGCCTGGAGTAAATGATGACACAAGGC
AATTGACCCACGCATGTATCTATCTCATTTTCTTACACCTTCTATTACCTTCTGCTCTCTCTGATTTGGAAAAAGCTGAA
AAAAAAGGTTGAAACCAGTTCCCTGAAATTATTCCCCTACTTGACTAATAAGTATATAAAGACGGTAGGTATTGATTGTA
ATTCTGTAAATCTATTTCTTAAACTTCTTAAATTCTACTTTTATAGTTAGTCTTTTTTTTAGTTTTAAAACACCAAGAAC
TTAGTTTCGACGGATACTAGTAAA-(SEQ ID NO: 162) followed by ATG . . . xySte2 . . .
TAG-followed by
CTCGAGACGGCTTTGAAAAAGTAATTTCGTGACCTTCGGTATAAGGTTACTACTAGATTCAGGTGCTCATCAGATGCACC
ACATTCTCTATAAAAAAAAATGGTATCTTTCTTATTTGATAATATTTAAACTCCTTTACATAATAAACATCTCGTAAGTA
GTGGTAGAAACCACCTTTGCTTTTACGAGTTCAAGCTTTTTTCTTGCCATGATCTAGAACTCTCAGGCAATATATACAGT
TAATCTTTTTTTACTGGGTTGTAGTTCTAATGTATTGTTTCGAAAAATAGCAACCAGGCACA (SEQ ID NO: 163)
STE2 locus with integrated TDH3-xy.Ste2-Ste2t expression cassette (100 bp
upstream and 100 bp downstream, corresponds to Ste2 terminator)
GTATCCTGCTTTGCAATGAAACAATAGTATCCGCTAAGAATTTAAGCAGGCCAACGTCCATACTGCTTAGGACCTGTGCC
TGGCAAGTCGCAGATTGAAG-AGTTT . . . (SEQ ID NO: 164) followed by TDH3p-xySte2 . . .
TAG-followed by
CTCGAGACGGCTTTGAAAAAGTAATTTCGTGACCTTCGGTATAAGGTTACTACTAGATTCAGGTGCTCATCAGATGCACC
ACATTCTCTATAAAAAAAAA (SEQ ID NO: 165)
STE3 locus with integrated TDH3-xy.Ste2-Ste2t expression cassette (100 bp
upstream and 100 bp downstream, corresponds to Ste2 terminator)
STE3 locus with integrated THD3p-xy.Ste2-Ste2t expression cassette (100 bp
upstream and 100 bp downstream, corresponds to Ste2 terminator)
CTATATTATTGTACCACATTGCCAGATTTATGAACTCTGGGTATGGGTGCTAATTTTCGTTAGAAGCGCTGGTACAATTT
TCTCTGTCATTGTGACACTA-AGTTT . . . (SEQ ID NO: 166) followed by THD3p-xySte2 . . .
TAG-followed by
CACAAGAGTGTCGCATTATATTTACTGGACTAGGAGTATTTTATTTTTACAGGACTAGGATTGAAATACTGCTTTTTAGT
GAATTGTGGCTCAAATAATG (SEQ ID NO: 167)

| Code | Codon optimized GPCR DNA sequence |
|---|---|
| Af | ATGAACTCCACCTTCGACCCATGGACCCAAAACATTACTTTGACTCAATCCGACGGTACCACTGTCATCTCCTCT |
| | TTGGCTTTGGCCGATGACTACTTGCACTACATGATTAGATTGGGTATCAACTACGGTGCCCAATTGGGTGCTTGT |
| | GCTGTTTTGTTGTTGGTTTTGTTATTGTTGACTAGACCAGAAAAGAGAGTTTCTTCTGTCTTCGTTTTGAACGTC |
| | GCTGCTTTGTTGGCTAACATCATCAGATTGGGTTGTCAATTGTCCTACTTCTCTACCGGTTTCGCTAGAATGTAC |
| | GCCTTGTTGGCCGGTGACTTCTCCAGAGTCTCTCGTGGTGCTTACGCCGGTCAAGTTATGGCCTCCGTCTTCTTC |
| | ACCATTGTCTTCATTTGTGTTGAAGCTTCTTTGGTTTTGCAAGTTCAAGTCGTCTGTTCTAACTTGAGAAGACAA |
| | TACAGAATCTTGTTATTGGGTGCTTCCACTTTGGCTGCCTTGGTTCCAATTGGTGTTCGTTTGACTTACTCCGTT |
| | TTAAACTGTATGGTTATTATGCACGCTGGTACTATGGACCACTTGGATTGGTTGGAATCTGCTACCAACATCGTT |
| | ACTACCGTTTCTATTTGTTTCTTCTGTGCTGTTTTCGTTGTCAAATTAGGTTTGGCTATCAAGATGAGAAAGCGT |
| | TTGGGTGTCAAACAATTCGGTCCAATGAGAGTTATCTTCATCATGGGTTGTCAAACCATGACCATCCCAGCTATT |
| | TTCGCTATTTGTCAATACTTCTCTAGAATTCCAGAATTTTCTCATAACGTTTTGACTTTGGTTATCATCTCTTTG |
| | CCATTGTCTTCTATCTGGGCCGGTTTTGCTTTGGTCCAAGCCAACTCTACCGCCAGATCTACCGAATCTAGACAT |
| | CATTTGTGGAACATTTTGTCTTCCGATGGTGCTACCAGAGACAAGCCATCCCAATGTGTTTCTTCTCCAATGACC |
| | TCTCCAACCACTACCTGTTACTCCGAACAATCCACCTCTAAGCCACAACAAGACCCAGAAAACGGTTTTGGTATT |
| | TCTGTTGCCCACGATATTTCCATCCACTCTTTCAGAAAGGACGCCCACGGTGATATT (SEQ ID NO: 168) |
| Ag | ATGGGTGAAGAGGTATCTAGCTTTGTGGAACAGTATTATGATCCAAACTATGATCCCAGTCAATCCATGCTAACC |
| | TACATGTCAAAGTTCAGTAACGAGTCGACAATAAAGTTTGAGGACTTACAAGAGTATATTAATGAAAACGTCATG |
| | TTGGGGGTATTTACTGGCGCAAAGATAGCGGCAGCAGCTCTGGCGTTGATAATCCTATGGATGGTGACTAAAAGG |
| | AAAAGGACACCCATTTACATCGTTAACCAGATATCACTCCTGCTTACAGTCATCCATGGCATTCTGGTGTTGTCT |
| | GGCTTGCTCGGGGGGTTTTCTTCTTCTATATTCACACTGACACTATTCCCTCAATGCGTGAATCGGAGTGATATT |
| | CGCCTGTTTGTCGCTACCAATATCTCCATGGTTTCGCTTATAGCCTCTATACAGGTTTCATTGGTTCTCCAAGTT |
| | CACGTAATCTTTCGAGCAGGCACTCACAGACGGTTAGGCATCTTCTTAACTGCGGTTTCCGCTATAATAGGGTTC |
| | ACAACCGTGTGCTTTTACCTGGTTTCTGCTGTCCTTTCAGTGATGGCTGTATACCAGGATATCGATAACATCGGC |
| | GATACATTCTTTCTGAGCATTGCGTACATTTGTATGGCCATATCTGTCAATTTCATTTTTTTGTTACTATCCGTT |
| | AAGCTGCTTCTTGCAATCAGATTAAGACGCTTCCTAGGTCTAAAACAATTTGATGGCTTACACATACTCTTCATT |
| | ATGTCTACTCAGACAATTATATGTCCGAGTATTCTGTTCATACTGGCTTTCGCTTGCGAGAAAAATATAACAGAT |
| | TCTTTGGTGTATATTGCGGTCTTACTCGTCTCACTGTCGCTACCACTGTCATCTGTGTGGGCAACAGCAGCCAAC |
| | AACGCAACAGTCCCACCTTTTTTGAACGCCCACTCTCTTACTTCTAGGTACAAAGCTGAATCCTGGTACACAGAT |
| | TCAAAGAATGATGCAGGTAGTTTTAGCTCCTCAGAAAATTGTGGATCGGGATATCGACATGGACGCTATTCTAAC |
| | AATGGGGGTAGTAGTCCACATCAATGTACGGGGGGGGATAATACCGTCATTGATATCGAAAAATGTCAATATAGA |
| | GTGAACCCTACGCCACATACTAGTGGGCAATTCGCTTTCAATCAGGATTCATTGGAAACTGAATTCTCGGAAGAT |
| | ACCGTCGTGCAAATTCGTACGCCCAATACTGAGGTTGAAGAGGAGGCCAAAATATTCTGGGCAAGAGCCAGTATC |
| | ACTCACGAAAATAGTTCTTCTGGCGTTGAGTGCGGTGCGCATGACATGCAAACCAACGTCTTCAAGACTCCTACA |
| | AGTCAAACCGGAAGTGATTGCAAC (SEQ ID NO: 169) |
| An | ATGGCTACCCACAACCAAATCTCTGATCAATGTCAATGGTCTTACCCAGAAGTCTTCACCACTCAAGCTGTCGAA |
| | GAACCAACCGCCGAACCAGCTTCTTACCACTTGCACTCTACCTTGACTATTATGGCTTCTAACTTCGACCCATGG |
| | AACCAAACCATTACCTTCAGATTGGAAGACGGTACTCCATTCGACATTTCTGTCGACTACTTGGACGGTATCTTG |
| | CAATACTCTATCAGAGCTTGTGTCAACTACGCTGCTCAATTGGGTGCTTCTGTCATTTTGTTTGTTATCTTGGTC |
| | TTGTTGACTAGAGCCGAAAAAAGAGCTTCTTGTTTGTTCTGGTTAAACTCCTTAGCTTTGTTGTTGAACTTCGCC |
| | AGATTGTTGTGTGACGTCTTGTTCTTCACCGGTAACTTCGTCAGAATTTACACTTTGATCTCCGCTGACGAATCT |
| | AGAGTTACTGCTTCCGACTTGGCTACTTCCATCGTCGGTGCTATCATGACCGCTTTGTTGTTGACCACTATTGAA |
| | ATTTCTTTGGTTTTGCAAGTCCAAGTCGTTTGTTCTAACTTGAGAAGAATCTACAGAAGAGCCTTGTTGTGTGTT |
| | TCCGCCGTCGTTGCCACTGCTACCATTGCTATTAGATACTCCTTGTTGGCTGTCAACATTAGAGCTATTTTGGAA |
| | TTCTCCGACCCAACTACTTACAACTGGTTGGAATCTTTAGCTACCGTCGCCTTGACCATCTCCATCTGTTACTTC |
| | TGTGTCATCTTCGTCACCAAGTTAGGTTTCGCTATTAGATTGAGAAGAAAGTTGGGTTTATCTGAATTGGGTCCA |
| | ATGAAGGTCGTCTTCATCATGGGTTGTCAAACCTTGGTCATCCCAGGTAAAAGAACCTTGTCTTCTTTGATTCCA |
| | CCAGTCATTGTTTCTATTACTCACTACGTCTCCGACGTCCCAGAATTGCAAACTAACGTTTTGACTATCGTCGCC |
| | TTGTCCTTGCCATTGTCCTCTATTTGGGCTGGTACCACCATTGACAAGCCAGTCACTCACTCTAACGTTAGAAAC |
| | TTGTGGCAAATCTTGTCCTTCTCTGGTTACAGACCAAAGCAATCTACCTACATTGCTACCACTACTACCGCTACT |
| | ACCAACGCTAAGCAATGTACCCACTGTTACTCTGAATCTAGATTGTTGACTGAAAAGGAATCTGGTCGTAACAAC |
| | GACACTTCTTCTAAGTCTTCCTCCCAATACGGTATCGCTGTCGAACACGATATTTCCGTTAGATCTGCTCGTCGT |
| | GAATCTTTTGACGTC (SEQ ID NO: 170) |
| Ao | ATGGACTCTAAGTTCGACCCATACTCTCAAAACTTGACTTTCCACGCTGCTGACGGTACCCCATTTCAAGTTCCA |
| | GTCATGACCTTGAACGACTTTTACCAATACTGTATTCAAATTTGTATCAACTACGGTGCTCAATTCGGTGCTTCC |
| | GTCATCATTTTCATTATCTTGTTGTTATTGACTAGACCAGACAAAAGAGCTTCTTCTGTTTTCTTCTTAAACGGT |
| | GGTGCCTTGTTGTTGAACATGGGTAGATTGTTGTGTCACATGATTTACTTCACTACTGACTTCGTCAAGGCTTAC |
| | CAATACTTCTCTTCTGATTACTCTAGAGCCCCAACCTCTGCCTACGCTAACTCCATTTTGGGTGTCGTCTTGACC |
| | ACCTTGTTGTTGGTTTGTATCGAAACCTCCTTGGTTTTACAAGTCCAAGTCGTCTGTGCTAACTTGAGACGTAGA |
| | TACAGAACCGTCTTATTGTGTGTTTCTATCTTGGTCGCCTTGATCCCAGTCGGTTTGAGATTGGGTTACATGGTT |
| | GAAAACTGTAAGACTATTGTTCAAACTGATACCCCATTGTCTTTGGTTTGGTTGGAATCTGCTACTAACATCGTC |
| | ATTACCATCTCCATCTGTTTCTTCTGTTCTATCTTCATCATCAAGTTGGGTTTCGCCATTCACCAAAGAAGAAGA |
| | TTGGGTGTCAGAGATTTCGGTCCAATGAAGGTCATTTTCGTCATGGGTTGTCAAACTTTGACTGTTCCAGCTTTG |
| | TTGTCTATTTTGCAATACGCTGTCTCTGTCCCAGAATTGAACTCTAACATTATGACTTTGGTTACTATCTCTTTG |
| | CCATTGTCCTCCATTTGGGCTGGTGTTTCTTTGACCCGTTCTTCCTCCACCGAAAACTCTCCATCCAGAGGTGCT |
| | TTGTGGAACCGTTTGACCGACTCTACCGGTACCAGATCTAACCAAACCTCTTCCACCGACACCGCCGTCGCTATG |
| | ACCTACCCATCTAACAAGTCTTCTACTGTCTGTTACGCCGATCAATCTTCTGTCAAGAGACAATACGATCCAGAA |
| | CAAGGTCACGGTATCTCTGTTGAACACGATGTTTCTGTCCACTCCTGTCAAAGATTG (SEQ ID NO: 171) |
| Bb | ATGGATGGTTCTTCTGCTCCATCTTCTCCAACTCCAGATCCAACCTTCGACAGATTCGCCGGTAACGTCACTTTC |
| | TTCTTGGCTGACCACATCACCACTACCTCCGTTCCAATGCCAGTCTTGAACGCCTACTACGACGAATCCTTGTGT |
| | ACTACCATGAACTACGGTGCTCAATTAGGTGCTTGTTTAGTTATGTTGGTTGTCGTTGTTGCTTTGACCCCAGCT |
| | GCTAAGTTGGCTAGAAGACCAGCTTCTGCTTTGCATTTGGTTGGTTTGTTGTTGTGTGCTGTTAGATCCGGTTTG |
| | TTGTTTGCTTACTTCGTCTCCCCAATCTCTCACTTTTACCAAGTTTGGGCTGGTGACTTCTCTGCCGTTTCCAGA |
| | AGATACTGGGACGCTTCTTTGGCTGCCAACACTTTAGCTTTCCCATTGGTTGTCGTCGTTGAAGCTGCTTTGATC |
| | AACCAAGCTTGGACCATGGTTGCTTTCTGGCCAAGAGCCGCTAAGGCCGCTGCCTGTGCTTGTTCTGCTGTCATT |
| | GTCTTGTTGACTATTGGTACTAGATTGGCCTACACTATCGTCCAAAACCACGCTATTGTTACTGCCGTCCCACCA |
| | GAACACTTCTTGTGGGCTATTCAATGGTCCGCTGTTATGGGTGCTGTTTCCATCTTCTGGTTTTGTGCCGTTTTC |
| | AACGTCAAGTTGGTCTGTCACTTAGTCGCTAACAGAGGTATCTTGCCATCTATCTCTGTTGTTAACCCAATGGAA |
| | GTCTTGGTTATGACTAACGGTACCTTGATGATTATCCCATCTATCTTCGCTGGTTTGGAATGGGCTAAGTTCACC |
| | AACTTCGAATCCGGTTCTTTGACTTTGACTTCCGTTATTATTATCTTGCCATTGGGTACTTTGGCTGCCCAACGT |
| | ATTTCTGGTCAAGGTTCCCAAGGTTACCAAGCTGGTCACTTATTCCACGAACAACAACAACAACAAGCTCGTACC |
| | CGTTCCGGTGCCTTCGGTTCCGCTTCTCAACAATCCCATCCAACTAACAAGGTTCCATCCTCTATTACCTTGTCT |
| | ACCTCTGGTACTCCAATTACTCCACAAATCTCTGCCGGTTCCCGTCCAGAATTACCATTGGTTGATAGATCCGAA |
| | CGTTTGGACCCAATTGACTTGGAATTGGGTAGAATCGATGCTTTCAGAGGTTCTTCCGACTTCTCTCCATCCACC |
| | GCTAGACCAAAGCGTATGCAACGTGATAACTTCGCC (SEQ ID NO: 172) |
| Bc | Sequence reported10 |
| | ATGGCTTCTAACTCTTCTAACTTCGACCCATTGACTCAATCTATCACTATCTTGATGGCTGACGGTATCACTACT |
| | GTTTCTTTCACTCCATTGGACATCGACTTCTTCTACTACTACAACGTTGCTTGTTGTATCAACTACGGTGCTCAA |
| | GCTGGTGCTTGTTTGTTGATGTTCTTCGTTGTTGTTGTTTTGACTAAGGCTGTTAAGAGAAAGACTTTGTTGTTC |
| | GTTTTGAACGTTTTGTCTTTGATCTTCGGTTTCTTGAGAGCTATGTTGTACGCTATCTACTTCTTGCAAGGTTTC |
| | AACGACTTCTACGCTGCTTTCACTTTCGACTTCTCTAGAGTTCCAAGATCTTCTTACGCTTCTTCTGTTGCTGGT |
| | TCTGTTATCCCATTGTGTATGACTATCACTGTTAACATGTCTTTGTACTTGCAAGCTTACACTGTTTGTAAGAAC |
| | TTGGACGACATCAAGAGAATCATCTTGACTACTTTGTCTGCTATCGTTGCTTTGTTGGCTATCGGTTTCAGATTC |
| | GCTGCTACTGTTGTTAACTCTGTTGCTATCTTGGCTACTTCTGCTTCTTCTGTTCCAATGCAATGGTTGGTTAAG |
| | GGTACTTTGGTTACTGAAACTATCTCTATCTGGTTCTTCTCTTTGATCTTCACTGGTAAGTTGGTTTGGACTTTG |
| | TACAACAGAAGAAGAAACGGTTGGAGACAATGGTCTGCTGTTAGAATCTTGGCTGCTATGGGTGGTTGTACTATG |
| | GTTATCCCATCTATCTTCGCTATCTTGGAATACGTTACTCCAGTTTCTTTCCCAGAAGCTGGTTCTATCGCTTTG |
| | ACTTCTGTTGCTTTGTTGTTGCCAATCTCTTCTTTGTGGGCTGGTATGGTTACTGACGAAGAAACTTCTGCTATC |
| | GACGTTTCTAACTTGACTGGTTCTAGAACTATGTTGGGTTCTCAATCTGGTAACTTCTCTAGAAAGACTCACGCT |
| | TCTGACATCACTGCTCAATCTTCTCACTTGGACTTCTCTTCTAGAAAGGGTTCTAACGCTACTATGATGAGAAAG |
| | GGTTCTAACGCTATGGACCAAGTTACTACTATCGACTGTGTTGTTGAAGACAACCAAGCTAACAGAGGTTTGAGA |
| | GACTCTACTGAAATGGACTTGGAAGCTATGGGTGTTAGAGTTAACAAGTCTTACGGTGTTCAAAAGGCTTAG |
| | (SEQ ID NO: 173) |
| Bm | ATGGCCTCAAACGGCTGGCAAAACAATGCAACATTTGATCCATATGCTCAGACGTTCGTGTTACTACAGCCAGAT |
| | GGTCTAACTCCATTCCCAGCGTTGCTAGGTGATGTTTTAGCTTTGAATACTGTCAGCGTTACCCAAGGTATTATT |
| | TATGGCACACAAGTCGGTATCTCCGGCTTGCTTTTACTGATACTATTGATTATGACTAAACCAGACAAGAGAAGA |
| | AGTTTGGTGTTCATCCTGAATAGTCTTTCTCTACTGTTGATCTTTGCCAGAAACGTGTTGAGTTGTGTGCAATTG |
| | ACTACTATATTTTATAACTTTTATAACTGGGAGTTGCACTGGTACCCTGAAAGCCCTGCATTATCAAGAGCTATG |
| | GATCTATCTGCCGCAACTGAAGTGTTAAATATACCAATAGACGTGGCCATCTTCTCATCCTTGGTAGTTCAAGTT |
| | CATATAGTTTGTTGCACGATACATACACTGGTGAGGACCTCAGCACTGTTATCTAGTGCCGCGGTTGGTCTGGCC |
| | GCTGTGGCTGTTAGATTTGCTCTGGCTGTGGTTAATATCAAATACAGTATTTTTGGTATTAATACATTGACTGAA |
| | CCCCAATTTAACTTAATAGTACACCTTAAAAGGGTAAGTGATATACTGACAGTGGTTGCTATCGCATTTTTCTCT |
| | AGCATTTTCGTCGCTAAGTTGGGAGTGGCGATTCACACTAGAAGAACGCTAAATTTAAAGAATTTCGGTGCTATT |
| | CAAATCATATTCATAATGGGATGTCAAACTATGTTGATTCCTTTAATATTTGTTATAGTGTCTTTCTATGCTTCT |
| | AGAGGATCTCAAATTGGGAGCATGGTTCCTACAGTGGTTGCAACCTTTTTGCCCCTATCAGGTATGTGGGCTAGC |
| | GCTCAAACGAATAACGAAAAAATGGGGAGGGCTGACCAACGTTTCCATCGTGCAGTCCCTGTGGGCGCGACTGAT |
| | TTCTCAGTGACTAAGGCTAGAAGCGCAAAAGCCAGTGACACTCTAGATACACTAATCGGTGACGAC |
| Ca | Sequence reported10 |
| | ATGAATATCAATTCAACTTTCATACCTGATAAACCAGGCGATATAATTATTAGTTATTCAATTCCAGGATTAGAT |
| | CAACCAATTCAAATTCCTTTCCATTCATTAGATTCATTTCAAACCGATCAAGCTAAAATAGCTTTAGTCATGGGG |
| | ATAACTATTGGGAGTTGTTCAATGACATTAATTTTTTTGATTTCTATAATGTATAAAACTAATAAATTAACAAAT |
| | TTAAAATTAAAATTAAAATTAAAATATATCTTGCAATGGATAAATCAAAAAATCTTCACCAAAAAAAGGAATGAC |
| | AACAAACAACAACAACAACAACAACAACAACAAATTGAATCATCATCATATAACAATACTACTACTACGCTGGGG |
| | GGTTATAAATTATTTTTATTTTATCTTAATTCATTGATTTTATTAATTGGTATTATTCGATCAGGTTGTTATTTA |
| | AATTATAATTTAGGTCCATTAAATTCACTTAGTTTTGTATTTACTGGTTGGTATGATGGATCATCATTTATATCA |
| | TCCGATGTAACTAATGGATTTAAATGTATTTTATATGCTTTAGTGGAAATTTCATTAGGTTTCCAAGTTTATGTG |
| | ATGTTCAAAACTTCAAATTTAAAAATTTGGGGGATAATGGCATCATTATTATCAATTGGTTTAGGATTGATTGTT |
| | GTTGCCTTTCAAATCAATTTAACAATTTTATCTCATATTCGATTTTCCCGGGCTATATCAACTAACAGAAGTGAA |
| | GAAGAATCATCATCATCATTATCATCTGATTCGGTTGGGTATGTGATTAATTCAATATGGATGGATTTACCAACA |
| | ATATTATTTTCCATTAGTATTAATATAATGACAATATTATTGATTGGTAAACTTATAATTGCTATTAGAACAAGA |
| | CGTTATTTAGGATTGAAACAATTTGATAGTTTCCATATTTTATTAATTGGTTTCAGTCAAACATTAATTATTCCT |
| | TCAATTATTTTGGTGGTTCATTATTTTTATTTATCACAAAATAAAGATTCTTTATTACAACAAATTAGTCTTTTA |
| | TTGATTATTTTAATGTTACCATTAAGTTCTTTATGGGCTCAAACTGCTAATAATACTCATAATATTAATTCATCT |
| | CCAAGTTTATCATTCATATCTCGTCATCATCTGTCTGATAGTAGTCGTAGTGGTGGTTCCAATACAATTGTTAGT |
| | AATGGTGGTAGTAATGGTGGTGGTGGTGGTGGTGGGAATTTCCCTGTTTCAGGTATTGATGCACAATTACCACCT |
| | GATATTGAAAAAATCTTACATGAAGATAATAATTATAAATTACTTAATAGTAATAATGAAAGTGTAAATGATGGA |
| | GATATTATCATTAATGATGAAGGTATGATTACTAAACAAATCACCATCAAAAGAGTGTAG |
| | (SEQ ID NO: 174) |
| Cau | ATGGAATTCACTGGTGACATCGTTTTGAAGTACACTTTGGGTGGTGAAGAATACTTGTCTACTTTCGAACAATTG |
| | GACTCTTCTGTTAACAGATCTTTGGAATTGGGTGTTGTTCACGGTATCGCTATCGCTTGTGGTGTTTTGTTGATG |
| | GTTTTGGCTTGGGTTATCATCATCAAGAAGAAGAACCCAATCTTCGTTTTGAACCAATTAACTTTACTATTGATG |
| | GTTATCAAGTCTTCTTTATACTTGGCTTTCTTGTTCGGTCCATTGTCTTCTTTGACTTACAAGTTCACTAGAGTT |
| | TTGCCACACGACAAGTGGCACGCTTTCCACGTTTACATCGCTACTAACGTTATCCACACTTTATTGATCGCTACT |
| | GTTGAAATGACTTTGGTCTTCCAAATCTACATCATTTTCAAGTCTCCAGAAGTTAGACACTTGGGTTACATCTTG |
| | ACTGGTGCTGCTTCTGCTTTGGCTCTAACTATCGTTGCTTTGTACATCCACTCTACTGTTATCTCTGCTGTTCAA |
| | TTAAAGGAACAATTGTTGATGCACGAAATCAAGATCACTAACTCTTGGGTTAACAACGTTCCAATCATTTTGTTC |
| | TCAGCTTCTTTGAACGTTGTTTGTATCATTTTGATCGCTAAGTTAGCTTTGGCTATCAAGACTAGAAGATACTTA |
| | GGTTTGAAGCAATTCGACGGTTTGCACATCTTGATGATCACTTCTACTCAAACTTTCATCGTTCCATCTGTTTTG |
| | ATGATCGTTAACTACAAGCAATCTTCTTCTTACTTGACTTTGTTGGCTAACATCTCTGTTATCTTGGTTGTCTGT |
| | AACTTGCCATTGTCTTCTTTGTGGGCTGCTTCTGCTAACAATTCTTCTACTCCAACTTCTTCTGCTAACACTGTT |
| | TTCTCTAGATGGGACTCTAAGTTCTCTGACACTGAAACTATCGCTCACGAATTACCATTGATCCCAGGTAAGGCT |
| | GAAAAGTTGCAATTGGTTTCTCCAATCACTGAAAAGGGTGACACTCACACTATGTGTGAATCTCACGGTGACCAA |
| | GACTTGATCGACAAGATGTTGGACGACATCGAAGGTGCTGTTATGACTACTGAATTCAACTTGAACAACAGAACT |
| | GTT (SEQ ID NO: 175) |
| Cc | ATGGCTGCTAGAATTATCCCAGCTTTGACCTTGACCGCCCCAACCTCTTACCCAACCGCCGGTGTTGGTGGTTAC |
| | TACTACGACACTGCTTTCGGTGTTCCAACCTACTCCTCTGCCGCTTTCAACCAAACCACCTGGAGATTGTTGGAT |
| | AACTGGGACCACATCAACGTCAACTACGCTTCTTCCGAAGGTTTGGCTGCTGGTTTAGGTTGGGCTACCTTGATT |
| | TACTTGTTGGCTTTGACTCCATCCCACAAGAGAACTACTCCATTCCACTGTTTCTTGTTGGTTGGTTTGATTTTC |
| | TTGTTGGGTCACTTGATGGTCAACATTATTGCCGCCTTGACCCCAGGTTTGAACACCACCTCTGCTTACACTTAC |
| | GTTACCTTGGATACCTCCTCTTCCGTCTGGCCACGTAAGTACATCGCTGTCTACGCTGTCAACGCTGTCGCTTCT |
| | TGGTTCGCTTTCATTTTTGCCACTATCTGTTTGTGGTTGCAAGCTAAAGGTTTAATGACCGGTATCAGAGTCCGT |
| | TTCATCATCGTCTACAAGATTATCTTGATGTACTTGATCGTTGCTGCTGTCATTGCTTTGGCTATCTGTATGGCT |
| | TTCAACATTCAACAAATCTTATACATTGGTAAGCCAGTTGAATTGGCTGACGGTACCGCTTTGTTGAGATTGAGA |
| | AACGCTTACTTAATCACCTACGCTATCTCTATTGGTTCTTTCTCCTTAGTTTCTATCTGTTCTATCATGGATATC |
| | ATCTGGAGAAGACCATCTAGAGTCATTAAGGGTCACAACATTTTCGCTTCCGCTTTGAACTTAGTTGGTTTGTTG |
| | TGTGCTCAATCCTTCGTCGTCCCATGTGAATACAAGAGAGCCTTGGGTCAAGTCCCAGATTGTACTACTTTCGCC |
| | GATCACATTTTCCACACCGTTATCTTCTGTATTTTGCAAGTTATTCCAAACTCTTCTGGTGTTATGTTGCCAGAA |
| | ATCATGTTATTGCCATCTGTTTACGTCATTTTGCCATTGGGTTCCTTGTTCATGACTGTTAACTCCCCAGAATCC |
| | GATGTCAACAAGACCTCTTTCCCACCAAAGTCCTCCCCAGGTCCATTCGACAGATCCCCAACTTTGACCTCTGGT |
| | ACCTTGCCAGGTTCTAGACCAGAATCCTACGTTTTGGATATGGCTTCTGACAAGAACTCCGGTAACAGAAAGTCT |
| | GTTTGTTCCCAATTCGACCGTGAATTGAACTTGATCGATTCTTTGGACACTTTGTCTGGTCGTGAAGGTGATTCT |
| | ATGTTGCACGCCCAATCCAACAACAACAACCAAACCAGAGAACAAGACAAGCAACCAAGAGCCGATACCACCCAC |
| | GTTGGTTCTGAAAACATGGTC (SEQ ID NO: 176) |
| Cg | Sequence reported10 |
| | ATGGAAATGGGTTACGACCCAAGAATGTACAACCCAAGAAACGAATACTTGAACTTCACTTCTGTTTACGACGTT |
| | AACGACACTATCAGATTCTCTACTTTGGACGCTATCGTTAAGGGTTTGTTGAGAATCGCTATCGTTCACGGTGTT |
| | AGATTGGGTGCTATCTTCATGACTTTGATCATCATGTTCATCTCTTCTAACACTTGGAAGAAGCCAATCTTCATC |
| | ATCAACATGGTTTCTTTGATGTTGGTTATGATCCACTCTGCTTTGTCTTTCCACTACTTGTTGTCTAACTACTCT |
| | TCTATCTCTTACATCTTGACTGGTTTCCCACAATTGATCACTTCTAACAACAAGAGAATCCAAGACGCTGCTTCT |
| | ATCGTTCAAGTTTTGTTGGTTGCTGCTATCGAAGCTTCTTTGGTTTTCCAAATCCACGTTATGTTCACTATCGAA |
| | AACATCAAGTTGATCAGAGAAATCGTTTTGTCTATCTCTATCGCTATGGGTTTGGCTACTGTTGCTACTTACTTG |
| | GCTGCTGCTATCAAGTTGATCAGAGGTTTGCACGACGAAGTTATGCCACAAACTCACTTGATCTTCAACTTGTCT |
| | ATCATCTTATTGGCTTCTTCTATCAACTTCATGACTTTCATCTTAGTTATCAAGTTGTTCTTCGCTATCAGATCT |
| | AGAAGATACTTAGGTTTGAGACAATTCGACGCTTTCCACATCTTGTTGATCATGTTCTGTCAATCTTTGTTGATC |
| | CCATCTGTTTTGTACATCATCGTTTACGCTGTTGACTCTAGATCTAACCAAGACTACTTGATCCCAATCGCTAAC |
| | TTGTTCGTTGTTTTGTCTTTGCCATTGTCTTCTATCTGGGCTAACACTTCTAACAACTCTTCTAGATCTCCAAAG |
| | TACTGGAAGAACTCTCAAACTAACAAGTCTAACGGTTCTTTCGTTTCTTCTATCTCTGTTAACTCTGACTCTCAA |
| | AACCCATTGTACAAGAAGATCGTTAGATTCACTTCTAAGGGTGACACTACTAGATCTATCGTTTCTGACTCTACT |
| | TTGGCTGAAGTTGGTAAGTACTCTATGCAAGACGTTTCTAACTCTAACTTCGAATGTAGAGACTTGGACTTCGAA |
| | AAGGTTAAGCACACTTGTGAAAACTTCGGTAGAATCTCTGAAACTTACTCTGAATTGTCTACTTTGGACACTACT |
| | GCTTTGAACGAAACTAGATTGTTCTGGAAGCAACAATCTCAATGTGACAAGTAG (SEQ ID NO: 177) |
| Cgu | ATGAAGTCCTGCTCCATCGGTTTCGGTATCCCATTCATTAATGAACCAAACTTCGAAACTGTTTCTATTTTGACC |
| | ATGGACGTTTCTTTCATTGACGCTGACGTCAATCCTGACAATATCTTGTTGAACTTCACCATTCCTGGTTACCAA |
| | AACGGTTTCTCTGTTCCAATGGTTGTTATTAACGAATTGCAAAAGTCTCAAATGAAATACGCTATTGTTTACGGT |
| | TGTGGTGTCGGTGCCTCCTTGATTTTGTTGTTTGTCGTCTGGATTTTGTGTTCTAGAAAGACTCCATTGTTTATC |
| | ATGAACAACATTCCATTAGTTTTGTACGTCATCTCCTCTTCTTTGAACTTGGCTTACATTACCGGTCCATTGTCT |
| | TCTGTTTCCGTCTTCTTGACCGGTATCTTGACTTCTCACGATGCCATTAACGTCGTTTACGCTTCCAACGCTTTG |
| | CAAATGTTGTTGATCTTTTCTATCCAATCTACCATGGCCTACCACGTTTACGTTATGTTCAAATCTCCACAAATT |
| | AAATACTTGAGATACATGTTAGTCGGTTTCTTGGGTTGTTTACAAATTGTCACCACCTGTTTATACATCAACTAC |
| | AATGTTTTGTACTCTCGTAGAATGCACAAATTGTACGAAACTGGTCAAACCTACCAAGATGGTACCGTTATGACT |
| | TTCGTTCCATTCATCTTGTTCCAATGTTCTGTCAACTTCTCTTCTATTTTCTTGGTTTTGAAGTTGATTATGGCC |
| | ATTAGAACCAGACGTTACTTGGGTTTGCGTCAATTCGGTGGTTTTCATATTTTGATGATCGTTTCTTTACAAACT |
| | ATGTTGGTCCCATCTATTTTGGTTTTGGTTAACTACGCCGCTCATAAGGCTGTTCCTTCCAACTTGTTATCTTCC |
| | GTTTCTATGATGATCATTGTTTTGTCTTTACCAGCTTCTTCTATGTGGGCCGCTGCTGCTAACGCCTCTTCTGCC |
| | CCTTCCTCCGCTGCTTCCTCCTTGTTCAGATACACCACTTCTGATTCCGATAGAACTTTGGAAACTAAATCTGAC |
| | CACTTCATCATGAAGCATGAGTCCCACAACTCTTCTCCAAATTCCTCCCCATTGACTTTGGTTCAAAAGAGAATT |
| | TCTGATGCCACCTTAGAATTACCAAAAGAGTTAGAAGACTTGATCGACTCCACCTCCATC |
| | (SEQ ID NO: 178) |
| Cl | ATGAACCCAGCTGACATCAACATCGAATACACCTTGGGTGATACTGCTTTCTCTTCCACTTTCGCTGATTTCGAA |
| | GCTTGGAAAACTAGAAACACTCAATTCGCTATTGTCAACGGTGTCGCTTTGGCTTGTGGTATTATCTTGATGGTC |
| | GTTTCTTGGATTATTATTGTTAACAAGAGAGCTCCAATCTTCGCTATGAACCAAACTATGTTGGTTATCATGGTT |
| | ATTAAGTCCGCTATGTACTTGAAGCATATCATGGGTCCATTGAACTCCTTGACCTTCCGTTTCACCGGTTTAATG |
| | GAAGAATCCTGGGCTCCATACAACGTTTACGTCACTATTAACGTCTTGCATGTTTTGTTGGTCGCTGCTGTCGAA |
| | TCCTCTTTGGTCTTCCAAATCCATGTTGTTTTCAAGTCTTCTAGAGCCAGAGTTGCTGGTAGAGCCATTGTTTCT |
| | GCTATGTCCACTTTGGCCTTGTTGATCGTTTCTTTGTACTTGTACTCTACTGTTAGACATGCTCAAACTTTGCGT |
| | GCTGAATTATCTCATGGTGACACTACCACTGTTGAACCATGGGTCGATAACGTTCCATTGATTTTGTTTTCCGCT |
| | TCTTTGAACGTTTTGTGTTTGTTGTTGGCCTTGAAATTGGTTTTCGCTGTCAGAACCAGAAGACATTTAGGTTTA |
| | AGACAATTCGACTCTTTCCACATCTTGATTATTATGGCCACTCAAACTTTCGTTATCCCATCCTCTTTGGTCATC |
| | GCTAACTACAGATACGCTTCTTCCCCATTGTTGTCTTCCATTTCCATCATCGTCGCCGTCTGTAACTTGCCATTG |
| | TGTTCCTTGTGGGCTTGTTCTAACAACAACTCTTCCTACCCAACTTCTTCTCAAAACACTATTTTGTCCAGATAC |
| | GAAACTGAAACCTCTCAAGCTACTGACGCTTCCTCTACCACCTGTGCCGGTATTGCTGAAAAGGGTTTCGACAAG |
| | TCTCCAGACTCTCCAACTTTCGGTGACCAAGACTCCGTCTCTATCTCCCATATCTTGGACTCTTTGGAAAAGGAT |
| | GTTGAAGGTGTCACCACCCATAGATTGACT (SEQ ID NO: 179) |
| Cn | ATGGACTCCTACTTGTTGAACCATCCAGGTGACATCTCTTTGAACTTCGCCTTGCCATTGTCCGATGAAGTCTAC |
| | ACTATTACCTTCAACGACTTAGACTCTCAATCTTCTTTTTCCATTCAATACTTGGTCATCCACTCTTGTGCCATT |
| | ACCGTCTGTTTGACCTTGTTGGTTTTGTTGAACTTGTTCATCAGAAACAAGAAGACTCCAGTCTTCGTTTTGAAC |
| | CAAGTCATCTTGTTCTTCGCTATCGTCAGATCTTCTTTGTTCATCGGTTTTATGAAGTCTCCATTGTCCACCATC |
| | ACCGCCTCTTTCACCGGTATCATTTCTGATGACCAAAAACACTTCTACAAGGTCTCCGTCGCTGCTAACGCCGCT |
| | TTGATCATTTTGGTCATGTTGATTCAAGTTTCTTTCACTTACCAAATCTACATTATTTTCAGATCCCCAGAAGTT |
| | AGAAAGTTCGGTGTCTTCATGACCTCCGCCTTGGGTGTCTTGATGGCTGTTACCTTCGGTTTTTACGTTAACTCC |
| | GCTGTCGCTTCTACCAAGCAATACCAACACATCTTCTACTCTACCGACCCATACATCATGGACTCTTGGGTCACT |
| | GGTTTGCCACCAATCTTGTACTCTGCTTCCGTCATCGCTATGTCTTTGGTCTTGGTTTTGAAGTTGGTCGCTGCT |
| | GTCAGAACCAGAAGATACTTGGGTTTGAAGCAATTCTCCTCCTACCACATCTTGTTGATTATGTTCACCCAAACC |
| | TTGTTCGTTCCAACCATCTTGACCATCTTAGCTTACGCTTTCTACGGTTACAACGATATCTTGATCCATATTTCT |
| | ACCACCATCACCGTTGTCTTGTTGCCATTCACCTCCATTTGGGCTTCTATCGCCAACAACTCTAGATCCTTGATG |
| | TCTGCCGCTTCCTTGTACTTCTCCGGTTCCAACTCCTCTTTGTCTGAATTGTCTTCTCCATCTCCATCTGATAAC |
| | GACACTTTGAACGAAAACGTCTTCGCCTTTTTTCCAGACAAGTTGCAAAAGATGAACTCTTCTGAAGCCGTTTCT |
| | GCTGTCGACAAGGTCGTTGTTCACGACCACTTTGATACCATCTCCCAAAAGTCTATCCCACACGACATCTTGGAA |
| | ATTTTGCAAGGTAACGAAGGTGGTCAAATGAAGGAACACATCTCTGTCTACTCTGATGACTCTTTCTCCAAGACT |
| | ACTCCACCAATTGTCGGTGGTAACTTGTTGATCACCAACACCGACATCGGTATGAAG (SEQ ID NO: 180) |
| Cp | ATGAACAAGATTGTCTCCAAGTTGTCTTCTTCTGACGTCATCGTTACCGTCACCATCCCAAACGAAGAAGATGGT |
| | ACTTACGAAGTCCCATTCTACGCTATTGACAACTACCACTACTCCCGTATGGAAAACGCTGTTGTTTTAGGTGCT |
| | ACCATTGGTGCTTGTTCTATGTTGTTGATCATGTTGATTGGTATTTTGTTCAAGAACTTCCAAAGATTGAGAAAG |
| | TCTTTGTTGTTCAACATCAACTTCGCTATCTTATTGATGTTGATTTTGAGATCCGCTTGTTACATCAACTACTTG |
| | ATGAACAACTTGTCTTCCATTTCTTTCTTCTTCACCGGTATTTTCGATGATGAATCTTTCATGTCTTCCGACGCT |
| | GCCAACGCCTTCAAGGTTATCTTGGTTGCCTTGATTGAAGTTTCCTTGACCTACCAAATTTACGTTATGTTCAAG |
| | ACCCCAATGTTGAAGTCCTGGGGTATTTTCGCCTCTGTCTTGGCCGGTGTTTTGGGTTTGGCTACTTTGGCTACC |
| | CAAATCTACACTACCGTTATGTCTCACGTTAACTTCGTCAACGGTACCACCGGTTCTCCATCTCAAGTTACTTCC |
| | GCTTGGATGGACATGCCAACTATCTTATTCTCCGTTTCTATTAACGTTTTGTCTATGTTCTTGGTTTGTAAGTTG |
| | GGTTTGGCCATCAGAACCAGACGTTACTTGGGTTTAAAGCAATTCGACGCTTTCCACATTTTATTCATTATGTCC |
| | ACTCAAACCATGATCATTCCATCCATCATCTTGTTCGTTCACTACTTCGATCAAAACGACTCTCAAACCACCTTG |
| | GTCAACATCTCTTTGTTATTGGTCGTCATTTCCTTGCCATTGTCTTCTTTGTGGGCTCAAACTGCTAACAACGTT |
| | AGAAGAATTGACACTTCTCCATCCATGTCCTTCATCTCTAGAGAAGCTTCCAACAGATCTGGTAACGAAACCTTG |
| | CACTCTGGTGCTACTATCTCTAAGTACAACACCTCCAACACCGTTAACACTACCCCAGGTACTTCTAAGGATGAC |
| | TCTTTGTTCATCTTGGACAGATCCATTCCAGAACAAAGAATTGTCGACACTGGTTTGCCAAAGGACTTGGAAAAG |
| | TTCATTAACAACGATTTTTACGAAGACGATGGTGGTATGATTGCCAGAGAAGTCACCATGTTGAAGACCGCTCAC |
| | ACAACCAA (SEQ ID NO: 181) |
| Ct | ATGGACATCAACAACACCATCCAATCTTCCGGTGACATCATCATTACCTACACCATCCCAGGTATCGAAGAACCA |
| | TTCGAATTGCCATTCGAAGTTTTGAACCACTTCCAATCTGAACAATCCAAGAACTGTTTGGTCATGGGTGTTATG |
| | ATCGGTTCTTGTTCCGTTTTGTTGATCTTCTTGGTCGGTATTTTGTTCAAAACCAACAAATTCTCTACTATTGGT |
| | AAGTCTAAGAACTTGTCTAAGAACTTCTTGTTCTACTTGAACTGTTTGATCACCTTCATCGGTATCATTCGTGCT |
| | GCCTGTTTTTCTAACTACTTGTTGGGTCCATTGAACTCTGCTTCTTTCGCTTTCACTGGTTGGTACAACGGTGAA |
| | TCTTACGCTTCTTCCGAAGCTGCTAACGGTTTCAGAGTCATCTTGTTCGCTTTGATTGAAACTTCTATGGTCTTC |
| | CAAGTTTTCGTTATGTTCAGAGGTGCTGGTATGAAAAAGTTGGCTTACTCCGTTACCATTTTGTGTACCGCTTTG |
| | GCTTTGGTCGTTGTTGGTTTCCAAATTAACTCCGCTGTCTTATCTCACAGAAGATTCGTCAACACCGTTAACGAA |
| | ATTGGTGATACTGGTTTGTCCTCCATTTGGTTGGACTTGCCAACCATCTTGTTCTCCGTCTCTGTCAACTTAATG |
| | TCTGTTTTGTTGATCGGTAAATTGATCATGGCTATTAAGACTAGAAGATACTTGGGTTTGAAACAATTCGATTCC |
| | TTCCACGTTTTGTTAATTTGTTCCACTCAAACTTTGTTGGTCCCATCTTTAATCTTGTTCGTTCACTACTTCTTG |
| | TTCTTTAGAAACGCCAACGTTATGTTGATTAACATTTCCATCTTGTTGATCGTCTTGATGTTGCCATTCTCTTCC |
| | TTGTGGGCTCAAACCGCCAACACCACCCAATACATCAACTCTTCCCCATCCTTCTCTTTCATCTCTAGAGAACCA |
| | TCTGCTAACTCTACTTTGCACTCCTCTTCCGGTCACTACTCTGAAAAGTCCTACGGTATTAACAAATTGAACACC |
| | CAAGGTTCTTCCCCAGCCACCTTAAAGGATGATCACAACTCCGTCATCTTGGAAGCTACCAACCCAATGTCTGGT |
| | TTCGACGCCCAATTGCCACCAGACATTGCTAGATTCTTGCAAGATGACATCAGAATTGAACCATCTTCTACCCAA |
| | GATTTCGTTTCCACTGAAGTCACCTACAAGAAGGTC (SEQ ID NO: 182) |
| Dh | ATGGACCACAACACCCAACACTTCAACAGACCTGAATACATTGAAATCCCAGTTCCACCATCTAAGGGTTTCAAC |
| | CCACACACCAACCCTGCTTTCTTCATCTACCCAGACGGTTCTAATATGACCTTTTGGTTCGGTCAAATCGACGAT |
| | TTCAGACGTGACCAATTATTCACTAACACCATCTTTTCCATTCAAATTGGTGCCGCTTTGGTCATCTTATGTGTC |
| | ATGTTTTGTGTTACCCACGCTGATAAGCGTAAAACCATTGTCTACTTGTTAAACGTTTCCAACTTGTTCGTTGTT |
| | ATCATTAGAGGTGTTTTCTTTGTTCATTACTTCATGGGTGGTTTGGCCAGAACCTATACCACTTTCACCTGGGAT |
| | ACTTCTGATGTTCAACAATCTGAGAAGGCTACTTCCATTGTCTCCTCTATTTGTTCTTTGATTTTGATGATCGGT |
| | ACTCAAATCTCCTTATTGTTGCAAGTCAGAATCTGTTACGCTTTGAACCCAAGATCCAAGACCGCTATCTTGGTT |
| | ACTTGTGGTTCTATTTCCGGTATTGCTACCACTGCTTATTTATTGTTGGGTGCTTACACTATTCAATTGAGAGAA |
| | AAGCCACCAGACATGAAGTTCATGAAGTGGGCTAAGCCAGTTGTTAACGCTTTGGTTGCCTTGTCCATTGTCTCC |
| | TTTTCTGGTATTTTCTCTTGGAGAATGTTCCAATCTGTCAGAAACAGAAGAAGAATGGGTTTCACTGGTATCGGT |
| | TCCTTGGAATCTTTGTTGGCTTCTGGTTTCCAATGTTTAGTCTTCCCTGGTTTGGTTACTACCGCTTTGACCGTC |
| | GCCGGTTCCACTTGGTATATCGCTGTTAACTTAACTACTCCATCTGACTTGACCGCTATTTACAACTGTTCCGCT |
| | TTTTTCGCTTATGCTTTCTCCATTCCATTGTTAAAGGAAAGAGCTCAAGTTGAAAAGACCATTTCTGTTGTCATT |
| | GCTATCGCTGGTGTCTTAGTCGTTGCTTACGGTGACGGTGCTGACGACGGTTCCACCTCTAACGGTGAAAAGGCT |
| | AGATTGGGTGGTAACGTCTTGATCGGTATCGGTTCTGTCTTGTATGGTTTATACGAAGTCTTGTATAAGAAGTTA |
| | TTATGTCCACCATCTGGTGCTTCCCCAGGTAGATCTGTTGTTTTCTCTAATACCGTTTGTGCTTGCATCGGTGCT |
| | TTCACTTTGTTATTCTTGTGGATCCCATTGCCATTGTTGCACTGGTCCGGTTGGGAAATTTTTGAATTGCCAACC |
| | GGTAAGACTGCTAAGTTATTGGGTATTTCCATTGCCGCTAACGCCACCTTCTCTGGTTCTTTCTTGATCTTAATT |
| | TCTTTGACTGGTCCAGTTTTGTCCTCTGTTGCCGCCTTGTTGACCATTTTCTTGGTTGCTATTACTGACAGAATT |
| | TTATTCGGTAGAGAATTGACTTCTGCTGCCATTTTGGGTGGTTTGTTGATCATCGCTGCCTTCGCTTTGTTATCT |
| | TGGGCTACTTGGAAGGAAATGATTGAAGAGAACGAGAAGGATACTATCGATTCCATCTCTGACGTTGGTGACCAC |
| | GATGAC (SEQ ID NO: 183) |
| Fg | Sequence reported10 |
| | ATGTCTAAGGAAGTTTTCGACCCATTCACTCAAAACGTTACTTTCTTCGCTCCAGACGGTAAGACTGAAATCTCT |
| | ATCCCAGTTGCTGCTATCGACCAAGTTAGAAGAATGATGGTTAACACTACTATCAACTACGCTACTCAATTGGGT |
| | GCTTGTTTGATCATGTTGGTTGTTTTGTTGGTTATGGTTCCAAAGGAAAAGTTCAGAAGACCATTCATGATCTTG |
| | CAAATCACTTCTTTGGTTATCTCTTGTTGTAGAATGTTGTTGTTGTCTATCTTCCACTCTTCTCAATTCTTGGAC |
| | TTCTACGTTTTCTGGGGTGACGACCACTCTAGAATCCCAAGATCTGCTTACGCTCCATCTGTTGCTGGTAACACT |
| | ATGTCTTTGTGTTTGGTTATCTCTGTTGAAACTATGTTGATGTCTCAAGCTTGGACTATGGTTAGATTGTGGCCA |
| | AACGTTTGGAAGTACATCATCGCTGGTGTTTCTTTGATCGTTTCTATCATGGCTATCTCTGTTAGATTGGCTTAC |
| | ACTATCATCCAAAACAACGCTGTTTTGAAGTTGGAACCAGCTTTCCACATGTTCTGGTTGATCAAGTGGACTGTT |
| | ATCATGAACGTTGCTTCTATCTCTTGGTGGTGTGCTATCTTCAACATCAAGTTGGTTTGGCACTTGATCTCTAAC |
| | AGAGGTATCTTGCCATCTTACAAGACTTTCACTCCAATGGAAGTTTTGATCATGACTAACGGTATCTTGATGATC |
| | ATCCCAGTTATCTTCGCTTCTTTGGAATGGGCTCACTTCGTTAACTTCGAATCTGCTTCTTTGACTTTGACTTCT |
| | GTTGCTGTTATCTTGCCATTGGGTACTTTGGCTGCTCAAAGAATCGCTTCTTCTGCTCCATCTTCTGCTAACTCT |
| | ACTGGTGCTTCTTCTGGTATCAGATACGGTGTTTCTGGTCCATCTTCTTTCACTGGTTTCAAGGCTCCATCTTTC |
| | TCTACTGGTACTACTGACAGACCACACGTTTCTATCTACGCTAGATGTGAAGCTGGTACTTCTTCTAGAGAACAC |
| | ATCAACCCACAAGGTGTTGAATTGGCTAAGTTGGACCCAGAAACTGACCACCACGTTAGAGTTGACAGAGCTTTC |
| | TTGCAAAGAGAAGAAAGAATCAGAGCTCCATTGTAG (SEQ ID NO: 184) |
| Gc | ATGGCCGAAGACTCCATCTTCCCAAACAACTCCACCTCTCCATTGACCAACCCAATTGTTGTTGAAACCATTAAG |
| | GGTACCGCTTACATTCCATTACACTACTTGGATGATTTGCAATACGAAAAGATGTTGTTGGCTTCCTTGTTCTCC |
| | GTTAGAATTGCTACTTCCTTCGTTGTTATTATTTGGTACTTCGTCGCTGTCAACAAGGCTAAGAGATCTAAGTTT |
| | TTGTACATTGTCAACCAAGTTTCTTTGTTGATCGTTTTTATCCAATCCATTTTGTCTTTGATTTACGTCTTCTCC |
| | AACTTCTCCAAGATGTCTACCATTTTGACCGGTGATTACACCGGTATCACTAAGAGAGACATTAACGTCTCTTGT |
| | GTTGCCTCCGTTTTCCAATTCTTGTTCATCGCTTGTATCGAATTGGCTTTGTTCATCCAAGCTACTGTCGTTTTC |
| | CAAAAATCTGTTAGATGGTTGAAGTTTTCCGTTTCTTTGATCCAAGGTTCCGTCGCTTTGACTACTACCGCCTTG |
| | TACATGGCCATTATTGTCCAATCCATCTACGCTACTTTGAACCCATACGCTGGTAACTTGATTAAAGGTCGTTTC |
| | GGTTACTTATTAGCTTCTTTGGGTAAGATTTTCTTCTCTATTTCTGTTACTTCTTGTATGTGTATCTTCGTTGGT |
| | AAGTTGGTCTTTGCTATTCACCAAAGAAGAACTTTGGGTATTAAGCAATTCGACGGTTTGCAAATTTTGGTCATT |
| | ATGTCTACTCAATCCATGATCATCCCAACTATTATCGTCTTGATGTCTTTTTTGAGACGTAACGCTGGTTCTGTT |
| | TACACCATGGCTACCTTGTTGGTCGCTTTGTCCTTGCCATTGTCCTCCTTGTGGGCTGAAGCCAAGACTACCAGA |
| | GACTCTGCTTCTTACACCGCTTACAGACCATCTGGTTCTCCAAACAACCGTTCTTTGTTCGCCATCTTCTCTGAT |
| | AGATTGGCTTGTGGTTCTGGTAGAAACAACAGACACGATGATGATTCTAGAGGTAACGGTTCTGTTAACGCCAGA |
| | AAGGCTGACGTCGAATCTACTATCGAAATGTCCTCTTGTTACACTGATTCCCCAACCTACTCCAAGTTCGAAGCT |
| | GGTTTGGACGCTAGAGGTATCGTCTTCTACAACGAACACGGTTTGCCAGTTGTCTCCGGTGAAGTTGGTGGTTCT |
| | TCCTCCAACGGTACTAAGTTGGGTTCTGGTCATAAGTACGAAGTCAACACTACTGTTGTTTTGTCTGATGTTGAC |
| | TCTCCATCTCCAACCGACGTCACCCGTAAG (SEQ ID NO: 185) |
| Hj | ATGTCTTCCTTCGACCCATACACTCAAAACATTACTATTTTGGTTTCTCCATCCTCTCCACCAATTTCCATTCCA |
| | ATCCCAGTTATCGACGCTTTCAACGACGAAACCGCTTCTATCATTACTAACTACGCCGCTCAATTAGGTGCTGCT |
| | TTGGCCATGTTATTAGTTTTGTTGGCCGCTACTCCAACCGCTAGATTGTTAAGAGCTGATGGTCCATCCTTGTTG |
| | CACGCTTTGGCCTTGTTAGTCTGTGTCGTCAGAACTGTCTTATTGATCTACTTCTTCTTGACCCCATTCTCTCAC |
| | TTCTACCAAGTCTGGACCGGTGACTTCTCTCAAGTTCCAGCTTGGAACTACAGAGCTTCTATTGCTGGTACCGTT |
| | TTGTCTACTTTGTTGACCGTTGTTACCGACGCTGCTTTGGTTAACCAAGCTTGGACTATGGTTTCTTTATTCGCT |
| | CCAAGAACTAAGAGAGCCGTTTGTGTTTTGTCCTTGTTAATCACCTTGTTGGCCATTTCTTTCAGAGTCGCTTAC |
| | ACCGTCATTCAATGTGAAGGTATCGCTGAATTGGCTGCTCCAAGACAATACGCTTGGTTGATCAGAGCCACTTTG |
| | ATCTTTAACATCTGTTCCATTGCCTGGTTCTGTGCTTTGTTCAACTCTAAGTTGGTTGCTCACTTGGTTACCAAC |
| | AGAGGTGTCTTGCCATCCCGTAGAGCCATGTCCCCAATGGAAGTTTTGATTATGGCCAACGGTATCTTGATGATT |
| | GTTCCAGTTGTTTTCGCTATCTTGGAATGGCACCACTTCATTAACTTCGAAGCTGGTTCTTTAACCCCAACCTCC |
| | ATCGCCATTATCTTGCCATTGTCCTCTTTGGCCGCCCAAAGAATCGCCAACACTTCTTCCTCT |
| | (SEQ ID NO: 186) |
| K1 | ATGTCAGAAGAGATACCCAGTTTGAACCCATTGTTCTACAATGAGACATATAATCCATTGCAGTCCGTCCTAACA |
| | TACAGTTCAATTTACGGAGATGGGACTGAAATAACATTTCAACAGCTACAAAATCTTGTCCATGAAAACATCACC |
| | CAAGCAATTATTTTTGGAACAAGGATCGGCGCTGCTGGATTAGCGTTGATTATAATGTGGATGGTCTCTAAGAAT |
| | AGAAAGACGCCGATATTCATAATAAATCAGAGTTCTTTGGTTCTTACAATTGTTCAATCTGCTTTATATCTATCA |
| | TATTTGTTGAGCAATTTTGGAGGAGTTCCCTTTGCTCTAACTTTGTTCCCACAGATGATAGGCGACCGTGACAAA |
| | CATCTTTACGGTGCCGTGACTCTAATTCAATGTCTATTGGTTGCGTGTATTGAGGTCTCGTTAGTCTTTCAGGTA |
| | AGAGTCATTTTCAAAGCAGATAGATATAGGAAGATAGGAATCATTTTGACTGGCGTCTCCGCTAGTTTTGGTGCT |
| | GCAACTGTAGCCATGTGGATGATTACTGCAATAAAATCTATTATTGTAGTGTATGATAGTCCATTGAACAAAGTT |
| | GACACATATTATTACAACATAGCAGTTATTTTACTTGCATGTTCAATAAATTTCATCACTCTTCTTCTATCAGTG |
| | AAACTTTTCCTGGCTTTCAGAGCTAGGAGACATTTAGGTTTGAAACAATTTGACTCATTTCACATTCTACTCATC |
| | ATGTCTACTCAGACATTAATAGGTCCATCGGTTTTGTATATTCTCGCCTACGCGCTGAACAATAAAGGAGTTAAG |
| | TCGTTGACTTCTATTGCTACATTGCTTGTAGTTCTTTCCCTACCTTTGACATCTATCTGGGCTGCTGCTGCAAAT |
| | GATGCACCAAGTGCCAGTACTTTCTATCGCCAATTCAACCCTTACTCTGCACAAAATCGTGATGATTCATCATCC |
| | TACTCTTATGGTAAAGCCTTTAGTGACAAATACTCTTTCAGTAACTCACCACAAACTTCGGATGGTTGTAGTTCA |
| | AAGGAACTTGAACTATCTACACAGTTGGAGATGGATTTAGAGTCTGGCGAATCTTTTATGGATAGAGCAAAAAGG |
| | TCCGATTTTGTTTCTTCTCCAGGATCAACAGATGCAACAGTGATTAAACAATTGAAAGCTTCCAACATCTATACC |
| | TCAGAAACAGATGCTGATGAAGAGGCAAGGGCATTTTGGGTGAATGCAATTCATGAAAACAAAGATGACGGTTTA |
| | ATGCAATCGAAAACCGTATTCAAAGAATTAAGA (SEQ ID NO: 187) |
| Kp | ATGGAAGAATACTCCGACTCCTTCGACCCATCCCAACAATTGTTGAACTTCACTTCCTTATACGGTGAAACCGAT |
| | GCTACTTTCGCTGAATTGGACGACTACCACTTCTACGTCGTTAAGTACGCCATCGTTTACGGTGCCAGAATTGGT |
| | GTCGGTATGTTTTGTACTTTGATGTTGTTCGTTGTTTCCAAGTCTTGGAAGACTCCAATCTTCGTCTTGAACCAA |
| | TCTTCTTTGATTTTGTTGATTATTCACTCCGGTTTCTACATCCACTACTTGACCAACCAATTCTCTTCCTTGACC |
| | TACATGTTCACTAGAATCCCAAACGAAACCCATGCTGGTGTCGATTTGCGTATTAACGTCGTTACCAACACCTTG |
| | TACGCTTTGTTGATCTTATCTATTGAAATTTCCTTAATTTACCAAGTCTTCGTTATCTTCAAAGGTGTCTACGAA |
| | AACTCTTTAAGATGGATTGTTACTATTTTCACCGCTTTATTCGCCGCCGCCGTCGTTGCTATTAACTTCTACGTC |
| | ACTACTTTGCAATCTGTCTCTATGTACAACTCTAACGTTGACTTTCCAAGATGGGCTTCTAACGTCCCATTGATC |
| | TTGTTCGCTTCTTCTGTCAACTGGGCTTGTTTGTTGTTGTCCTTGAAGTTGTTCTTCGCTATCAAGGTTAGAAGA |
| | TCTTTGGGTTTGAGACAATTCGACACTTTTCACATCTTGGCCATCATGTTCTCTCAAACTTTGATTATCCCATCC |
| | ATTTTGATTGTCTTGGGTTACACTGGTACCAGAGACAGAGACTCCTTGGCTTCTTTGGGTTTCTTGTTGATCGTT |
| | GTTTCTTTGCCATTTTCCTCTATGTGGGCTGCCACTGCTAACAACTCCAACATCCCAACCTCTACCGGTTCTTTC |
| | GCCTGGAAGAACAGATACTCCCCATCTACTTACTCCGACGATACCACTGCTGTTTCCAAGTCCTTCACTATTATG |
| | ACCGCTAAGGATGAATGTTTCACCACTGATACCGAAGGTTCTCCAAGATTCATCAAGGGTGACAGAACCTCCGAA |
| | GATTTGCACTTC (SEQ ID NO: 188) |
| Le | Sequence reported10 |
| | ATGGACGAAGCAATCAATGCAAACCTTGTTTCTGGAGATATTATAGTCTCTTTTAACATTCCTGGTTTGCCAGAA |
| | CCGGTACAAGTGCCATTCAGCGAATTTGATTCGTTTCATAAAGACCAGCTCATTGGAGTCATCATTCTTGGAGTC |
| | ACTATTGGAGCATGCTCGCTTTTGTTGATATTGCTACTTGGAATGTTATACAAGAGCCGTGAAAAGTATTGGAAA |
| | TCACTATTATTTATGCTCAATGTATGCATCTTGGCTGCCACAATCTTAAGGAGCGGTTGCTTCTTAGACTATTAT |
| | CTAAGTGATTTGGCCAGTATCAGTTATACATTTACTGGAGTATACAATGGTACCAGCTTTGCTAGCTCTGACGCG |
| | GCAAATGTGTTCAAGACTATTATGTTTGCCTTGATTGAAACTTCGTTAACCTTTCAAGTGTATGTCATGTTTCAA |
| | GGGACCACTTGGAAAAATTGGGGCCATGCTGTCACTGCATTATCGGGTCTCTTGTCTGTTGCCTCAGTGGCGTTC |
| | CAGATCTACACCACGATTTTATCCCACAATAATTTCAATGCTACAATCTCGGGAACCGGTACATTAACTTCAGGT |
| | GTTTGGATGGACTTACCAACACTCTTGTTTGCCGCAAGTATCAATTTTATGACCATTTTGTTGTTATTTAAGTTG |
| | GGAATGGCCATTAGACAAAGAAGGTATTTAGGTTTAAAACAGTTTGATGGGTTCCATATCTTATTCATCATGTTT |
| | ACCCAAACATTGTTCATACCCTCGATTTTGCTTGTGATCCACTACTTTTACCAGGCAATGTCTGGACCATTCATC |
| | ATCAACATGGCGTTGTTCTTGGTGGTGGCATTCTTGCCATTGAGTTCATTATGGGCACAAACTGCAAACACTACT |
| | AAAAAGATTGAATCTTCGCCAAGTATGAGCTTTATTACTAGACGAAAATCAGAGGATGAGTCACCACTGGCTGCT |
| | AACGACGAGGATAGGTTACGAAAATTCACCACAACTTTGGATTTGTCGGGCAACAAGAACAATACAACAAACAAT |
| | AATAACAATAGCAACAACATTAACAACAATATGAGCAACATCAACTACCCTTCTACAGGACTGGGAGAAGACGAT |
| | AAATCCTTTATATTTGAGATGGAACCCAGTCGGGAAAGAGCTGCAATAGAAGAGATTGATCTTGGAGCAAGGATC |
| | GATACCGGTTTGCCCAGAGATTTAGAGAAATTTCTAGTTGATGGGTTTGACGATAGTGATGACGGAGAAGGAATG |
| | ATAGCCAGAGAAGTGACTATGTTGAAAAAATAG (SEQ ID NO: 189) |
| Mg | ATGGTGGTAACAGCTCCACCTTCAGTTGACAGAACATATTTTATCCCGAATTCTACCTTTGATCCATATCAACAA |
| | GACTTGACGTTGGTCTATCCCGATGGTGTGCACGCCCTGGTTGCTAACGTTGATGATATAGTGTACTTCATGGGT |
| | CTAGCAGTTAAGTCTACGCTAATATTTGCTATTCAAATTGGTATTTCATTTGTATTAATGTTGGTTATTGCCCTG |
| | TTGACGAAACCTGAAAGAAGAGTTACGTTGGTATTCTTCTTAAACATGACTGCACTTTTTACCATCTTCATCAGA |
| | GCCATATTGATGTGTACTACATTTGTTGGTACATATTACAATTTTTACAACTGGATTATGGGCAACTACCCGAAC |
| | TCTGGTTTAGCTGATCGTGTATCTATTGCAGCCGAAGTTTTTGCTTTTCTGATTATACTGTCATTAGAACTTTCT |
| | ATGATGTTTCAAGTTCGTATTGTATGCATCAACCTGAGCTCATTCAGGAGGAGAATAATTACTTTTAGTAGTATA |
| | GTGGTTGCAATGATTGTTTGTACAGTTAGATTTGCCCTTATGGTGTTGTCTTGTGATTGGAGGATTGTGAATATC |
| | GGAGATGCGACGCAAGAAAAGAACAGAATCATTAACCGTGTGGCATCCGGTTATAACATATGCACAATAGCATCA |
| | ATCATTTTTTTCAACACCATCTTCGTCTCCAAGTTGGCCGTCGCTATCAAACATCGTAGAAGCATGGGCATGAAA |
| | CAATTCGGTCCAATGCAGATCATCTTTGTTATGGGTTGTCAAACGCTTCTAATTCCAGCCATCTTTGGAATTATA |
| | TCTTACTTTGCTCTAGCTAGCACTCAGGTCTACTCTTTAATGCCAATGGTCGTAGCTATCTTCTTACCATTAAGT |
| | TCTATGTGGGCTAGTTTTAACACCAACAAAACCAACAGTGTTACAAATATGAGGCAACCAAACGTCTATAGGCCT |
| | AATATGATCATCGGTCAAGACACAACCCAAAATTCCGGAAAGAATACAAACATAAGTGGTACGTCAAACTCCACG |
| | GCAACTACAAGTAGTTTTGCTAGCGATAAGAGACGTCTAAATTTATCTTTCAATACACAAGGTACACTGGTTAAT |
| | TCAATAAGTGAAGAAGAGGTTAATAACCCACAAAAATTGGGTCCTTCCGCTACCGTTGCGGTAATGGATAGAGAT |
| | TCTTTGGAATTAGAGATGAGACAACACGGCATCGCTCAAGGTAGGTCATACTCAGTCCGTTCCGAC |
| | (SEQ ID NO: 190) |
| Mo | Sequence reported10 |
| | ATGGACCAAACTTTGTCTGCTACTGGTACTGCTACTTCTCCACCAGGTCCAGCTTTGACTGTTGACCCAAGATTC |
| | CAAACTATCACTATGTTGACTCCAGCTTTGATGGGTCAAGGTTTCGAAGAAGTTCAAACTACTCCAGCTGAAATC |
| | AACGACGTTTACTTCTTGGCTTTCAACACTGCTATCGGTTACTCTACTCAAATCGGTGCTTGTTTCATCATGTTG |
| | TTGGTTTTGTTGACTATGACTGCTAAGGCTAGATTCGCTAGAATCCCAACTATCATCAACACTGCTGCTTTGGTT |
| | GTTTCTATCATCAGATGTACTTTGTTGGTTATCTTCTTCACTTCTACTATGATGGAATTCTACACTATCTTCTCT |
| | GACGACTTCTCTTTCGTTCACCCAAACGACATCAGAAGATCTGTTGCTGCTACTGTTTTCGCTCCATTGCAATTG |
| | GCTTTGGTTGAAGCTGCTTTGATGGTTCAAGCTTGGGCTATGGTTGAATTGTGGCCAAGAGCTTGGAAGGTTTCT |
| | GGTATCGCTTTCTCTTTGATCTTGGCTACTGTTACTGTTGCTTTCAAGTGTGCTTCTGCTGCTGTTACTGTTAAG |
| | TCTGCTTTGGAACCATTGGACCCAAGACCATACTTGTGGATCAGACAAACTGACTTGGCTTTCACTACTGCTATG |
| | GTTACTTGGTTCTGTTTCTTGTTCAACGTTAGATTGATCATGCACATGTGGCAAAACAGATCTATCTTGCCAACT |
| | GTTAAGGGTTTGTCTCCAATGGAAGTTTTGGTTATGGCTAACGGTTTGTTGATGGTTTTCCCAGTTTTGTTCGCT |
| | GGTTTGTACTACGGTAACTTCGGTCAATTCGAATCTGCTTCTTTGACTATCACTTCTGTTGTTTTGGTTTTGCCA |
| | TTGGGTACTTTGGTTGCTCAAAGATTGGCTGTTAACAACACTGTTGCTGGTTCTTCTGCTAACACTGACATGGAC |
| | GACAAGTTGGCTTTCTTGGGTAACGCTACTACTGTTACTTCTTCTGCTGCTGGTTTCGCTGGTTCTTCTGCTTCT |
| | GCTACTAGATCTAGATTGGCTTCTCCAAGACAAAACTCTCAATTGTCTACTTCTGTTTCTGCTGGTAAGCCAAGA |
| | GCTGACCCAATCGACTTGGAATTGCAAAGAATCGACGACGAAGACGACGACTTCTCTAGATCTGGTTCTGCTGGT |
| | GGTGTTAGAGTTGAAAGATCTATCGAAAGAAGAGAAGAAAGATTGTAG (SEQ ID NO: 191) |
| Nc | ATGGCGTCCTCTTCCTCACCACCTGCAGACATTTTCTCAGGGATCACGCAATCACTAAATAGTACACACGCGACG |
| | CTTACACTACCGATTCCGCCAGCGGACAGGGATCATCTGGAAAATCAAGTATTATTTTTGTTTGACAATCACGGT |
| | CAGTTACTTAATGTAACTACAACTTACATTGACGCTTTTAACAATATGCTGGTCTCTACTACTATAAACTATGCA |
| | ACGCAAATTGGAGCTACTTTTATAATGCTAGCCATTATGTTATTAATGACTCCCAGAAGGAGGTTCAAACGTTTA |
| | CCAACAATTATTAGCTTGTTAGCCTTATGTATTAATTTGATCAGGGTGGTTTTGCTGGCCCTGTTTTTTCCTTCT |
| | CACTGGACAGACTTCTACGTGTTGTATTCCGGTGACTGGCAGTTTGTACCTCCAGGGGATATGCAAATATCTGTT |
| | GCTGCTACGGTTTTGTCTATCCCAGTGACGGCATTATTATTGAGCGCATTGATGGTTCAAGCCTGGTCAATGATG |
| | CAATTATGGACACCACTGTGGAGGGCACTAGTGGTACTAGTGTCCGGGCTATTGTCACTGGTAACTGTGGCAATG |
| | AGTTTCGCGAATTGCATTTTCCAAGCGAAAAATATTTTGTATGCCGACCCTTTACCCTCCTACTGGGTCAGAAAA |
| | TTGTACTTAGCATTAACGACTGGGTCTATAAGTTGGTTCACATTCCTTTTTATGATAAGATTGGTTATGCATATG |
| | TGGACAAACAGATCTATATTACCAAGCATGAAGGGTTTGAAGGCTATGGATGTATTGATTATTACGAATTCTATA |
| | TTGATGTTAATCCCAGTGTTGTTTGCAGGCTTGGAATTTCTGGATAGTGCCTCTGGATTTGAGTCCGGGTCTTTG |
| | ACTCAAACCTCTGTAGTGATTGTCCTGCCTTTGGGTACTTTAGTAGCACAAAGAATAGCTACGAGGGGTTACATG |
| | CCCGATAGTCTGGAGGCTTCTAGCGGACCAAATGGTTCATTGCCGTTATCTAATTTAAGTTTCGCTGGAGGGGGC |
| | GGTGGTGGTTCTGGGGGACATAAAGATAAAGAAAACGGTGGCGGTATTATACCGCCTACTACGAACAATACTGCT |
| | GCTACTAATTTTTCTTCATCAATCGCGTGTTCTGGTATATCTTGTTTACCAAAAGTCAAAAGAATGACCGCGAGT |
| | TCAGCCTCAAGTAGCCAGAGACCGTTGTTGACAATGACTAACTCAACCATAGCGAGTAATGACAGTTCAGGTTTC |
| | CCTTCTCCTGGCATACATAATACCACTACTACGACAACACAATACCAATATTCCATGGGAATGAACATGCCGAAC |
| | TTTCCTCCAGTCCCGTTCCCAGGTTACCAGTCACGTACTACCGGTGTTACTTCCCATATTGTGTCCGACGGTAGA |
| | CATCACCAGGGTATGAACAGGCACCCATCTGTTGACCATTTTGATAGGGAACTTGCTAGGATTGATGATGAAGAT |
| | GACGATGGTTACCCTTTCGCATCAAGTGAAAAGGCCGTTATGCACGGAGACGATGACGACGATGTGGAAAGGGGA |
| | CGTCGTAGAGCTCTACCACCATCCTTAGGTGGAGTTAGAGTTGAAAGGACGATCGAGACCAGGAGCGAGGAACGT |
| | ATGCCATCTCCGGACCCATTGGGTGTTACGAAGCCTAGATCATTCGAG (SEQ ID NO: 192) |
| Pb | Sequence reported10 |
| | ATGGCACCCTCATTCGACCCCTTCAACCAAAGCGTGGTCTTCCACAAGGCCGACGGAACTCCATTCAACGTCTCA |
| | ATCCATGAACTAGACGACTTCGTGCAGTACAACACCAAAGTCTGCATCAACTACTCTTCCCAGCTCGGAGCATCT |
| | GTCATTGCAGGACTCATGCTTGCCATGCTGACACACTCAGAAAAGCGTCGTCTGCCAGTTTTCTTCCTAAACACA |
| | TTCGCACTGGCCATGAACTTTGCCCGCCTGCTCTGCATGACCATCTACTTCACCACGGGCTTCAACAAGTCCTAT |
| | GCCTACTTTGGTCAGGATTACTCCCAGGTGCCTGGGAGCGCCTACGCAGCCTCTGTCTTGGGCGTTGTCTTCACC |
| | ACTCTCCTGGTAATCAGCATGGAAATGTCCCTCCTGATCCAAACAAGGGTTGTCTGCACGACCCTTCCGGATATC |
| | CAACGTTATCTACTCATGGCAGTTTCCTCCGCGATTTCCCTGATGGCCATCGGGTTCCGCCTTGGCTTAATGGTT |
| | GAGAACTGCATTGCCATTGTGCAGGCGTCGAATTTCGCCCCTTTTATCTGGCTTCAAAGCGCCTCGAACATCACC |
| | ATTACGATCAGCACATGTTTCTTCAGTGCCGTCTTTGTTACGAAATTGGCATATGCACTCGTCACTCGTATACGA |
| | CTAGGCTTGACGAGGTTTGGTGCTATGCAGGTTATGTTCATCATGTCCTGCCAGACTATGGTGATTCCAGCCATC |
| | TTCTCAATTCTCCAATACCCACTCCCCAAGTACGAAATGAACTCCAACCTCTTTACGCTGGTGGCCATTTTCCTC |
| | CCTCTTTCCTCGCTATGGGCTTCAGTTGCTACGAGATCCAGTTTCGAGACGTCTTCTTCCGGCCGCCATCAGTAT |
| | CTTTGGCCAAGCGAACAGAGCAATAACGTCACCAATTCGGAAATTAAGTATCAGGTCAGCTTCTCTCAGAACCAC |
| | ACTACGTTGCGGTCTGGAGGGTCTGTGGCCACGACACTCTCCCCGGACCGGCTCGACCCGGTTTATTGTGAAGTT |
| | GAAGCTGGCACAAAGGCCTAG (SEQ ID NO: 193) |
| Pd | ATGTCCACTGCCAACGTTCATTTACCAGCTGATTTCGATCCAACTAGACAAAACATCACTATCTATACCCCAGAC |
| | GGTACCCCAGTTGTTGCTACCTTGCCAATGATCAATTTGTTTAACAGACAAAACAACGAAATCTGTGTTGTTTAC |
| | GGTTGTCAATTGGGTGCCTCTTTAATTATGTTCTTGGTTGTTTTGTTGACCACCAGAGTTTCCAAGAGAAAATCT |
| | CCAATCTTCGTCTTGAACGTTTTGTCTTTGATTATTTCTTGTTTAAGATCCTTGTTGCAAATTTTATACTATATT |
| | GGTCCATGGACCGAGATCTACAGATACTTGTCTTTCGATTACTCTACTGTCCCAGCTTCCGCTTACGCTAATTCT |
| | GTTGCTGCCACTTTATTAACCTTATTCTTATTGATTACCATTGAAGCTTCTTTAGTTTTACAAACTAACGTTGTC |
| | TGCAAGTCTATGTCTTCTCACATTCGTTGGCCAGTTACTGCTTTGTCCATGGTTGTCTCTTTATTGGCTATTTCT |
| | TTTAGATTCGGTTTGACCATCCGTAACATCGAAGGTATCTTAGGTGCTACTGTCAAATCCGACTCCTTAATGTTC |
| | TCTGGTGCCTCTTTGATCTCTGAAACTGCTTCTATCTGGTTCTTCTGCACTATTTTCGTTATTAAATTGGGTTGG |
| | ACCTTGTACCAAAGAAAGAAGATGGGTTTGAAGCAATGGGGTCCAATGCAAATTATCACTATCATGGCTGGTTGC |
| | ACCATGTTGATCCCATCCTTGTTCACTGTTTTGGAATTCTTCCCTGAAGAAACTTTCTACGAGGCCGGTACTTTG |
| | GCTATCTGTTTGGTTGCTATTTTGTTGCCATTATCTTCCGTCTGGGCTGCCGCTGCTATTGATGGTGATGAACCA |
| | GTCCGTCCACATGGTTCTACCCCAAAATTCGCTTCTTTCAACATGGGTTCCGACTACAAATCTTCTTCTGCTCAC |
| | TTGCCAAGATCTATTAGAAAGGCCTCCGTCCCAGCTGAACATTTATCTAGAACTTCTGAAGAAGAGTTAGGTGAC |
| | GACGGTACTTTGAACAGAGGTGGTGCCTACGGTATGGACAGAATGTCCGGTTCTATCTCCCCTAGAGGTGTCAGA |
| | ATTGAAAGAACTTACGAAGTTCATACCGCTGGTAGAGGTGGTTCTATCGAGAGAGAGGACATCTTC |
| | (SEQ ID NO: 194) |
| Pr | ATGGCTACCTCTTCCCCAATCCAACCATTTGACCCATTCACCCAAAACGTTACCTTCCGTTTGCAAGACGGTACC |
| | GAATTCCCAGTTTCTGTCAAGGCTTTGGACGTCTTCGTCATGTACAACGTTAGAGTCTGTATTAACTACGGTTGT |
| | CAATTCGGTGCCTCCTTCGTCTTGTTAGTCATTTTAGTCTTGTTAACTCAATCCGACAAGAGAAGATCTGCTGTC |
| | TTCATTTTGAACGGTTTGGCTTTGTTCTTGAACTCTTCTAGATTGTTGTTTCAAGTTATTCACTTCTCCACTGCC |
| | TTCGAACAAGTCTACCCATACGTCTCTGGTGACTACTCCTCTGTCCCATGGTCCGCTTACGCTATCTCCATTGTC |
| | GCTGTTGTTTTGACTACCTTGGTCGTTGTTTGTATCGAAGCTTCTTTGGTTATTCAAGTTCACGTTGTCTGTTCC |
| | ACCTTGAGACGTAGATACAGACACCCATTATTAGCTATTTCTATTTTGGTCGCTTTGGTTCCAATCGGTTTCAGA |
| | TGTGCTTGGATGGTCGCTAACTGTAAGGCTATTATTAAATTGACCTACACCAACGACGTTTGGTGGATCGAATCT |
| | GCTACTAACATCTGTGTCACTATCTCCATCTGTTTCTTCTGTGTTATCTTCGTTACCAAGTTGGGTTTCGCCATC |
| | AAGCAAAGAAGAAGATTGGGTGTTAGAGAATTCGGTCCAATGAAGGTTATTTTCGTCATGGGTTGTCAAACTATG |
| | GTTGTTCCAGCTATTTTCTCCATCACCCAATACTACGTCGTCGTCCCAGAATTCTCCTCTAACGTCGTTACTTTG |
| | GTTGTCATTTCTTTACCATTATCTTCCATTTGGGCCGGTGCTGTCTTGGAAAACGCTAGAAGAACCGGTTCCCAA |
| | GATAGACAAAGAAGACGTAACTTGTGGAGAGCTTTGGTTGGTGGTGCTGAATCCTTGTTATCCCCAACTAAGGAC |
| | TCTCCAACCTCTTTGTCTGCTATGACTGCTGCTCAAACCTTATGTTACTCTGATCACACCATGTCCAAGGGTTCT |
| | CCAACTTCCAGAGACACCGATGCTTTCTACGGTATCTCCGTTGAACACGACATCTCCATTAACAGAGTTCAACGT |
| | AACAACTCCATCGTC (SEQ ID NO: 195) |
| Sc | Sequence reported10 (SEQ ID NO: 196) |
| | ATGTCTGATGCGGCTCCTTCATTGAGCAATCTATTTTATGATCCAACGTATAATCCTGGTCAAAGCACCATTAAC |
| | TACACTTCCATATATGGGAATGGATCTACCATCACTTTCGATGAGTTGCAAGGTTTAGTTAACAGTACTGTTACT |
| | CAGGCCATTATGTTTGGTGTCAGATGTGGTGCAGCTGCTTTGACTTTGATTGTCATGTGGATGACATCGAGAAGC |
| | AGAAAAACGCCGATTTTCATTATCAACCAAGTTTCATTGTTTTTAATCATTTTGCATTCTGCACTCTATTTTAAA |
| | TATTTACTGTCTAATTACTCTTCAGTGACTTACGCTCTCACCGGATTTCCTCAGTTCATCAGTAGAGGTGACGTT |
| | CATGTTTATGGTGCTACAAATATAATTCAAGTCCTTCTTGTGGCTTCTATTGAGACTTCACTGGTGTTTCAGATA |
| | AAAGTTATTTTCACAGGCGACAACTTCAAAAGGATAGGTTTGATGCTGACGTCGATATCTTTCACTTTAGGGATT |
| | GCTACAGTTACCATGTATTTTGTAAGCGCTGTTAAAGGTATGATTGTGACTTATAATGATGTTAGTGCCACCCAA |
| | GATAAATACTTCAATGCATCCACAATTTTACTTGCATCCTCAATAAACTTTATGTCATTTGTCCTGGTAGTTAAA |
| | TTGATTTTAGCTATTAGATCAAGAAGATTCCTTGGTCTCAAGCAGTTCGATAGTTTCCATATTTTACTCATAATG |
| | TCATGTCAATCTTTGTTGGTTCCATCGATAATATTCATCCTCGCATACAGTTTGAAACCAAACCAGGGAACAGAT |
| | GTCTTGACTACTGTTGCAACATTACTTGCTGTATTGTCTTTACCATTATCATCAATGTGGGCCACGGCTGCTAAT |
| | AATGCATCCAAAACAAACACAATTACTTCAGACTTTACAACATCCACAGATAGGTTTTATCCAGGCACGCTGTCT |
| | AGCTTTCAAACTGATAGTATCAACAACGATGCTAAAAGCAGTCTCAGAAGTAGATTATATGACCTATATCCTAGA |
| | AGGAAGGAAACAACATCGGATAAACATTCGGAAAGAACTTTTGTTTCTGAGACTGCAGATGATATAGAGAAAAAT |
| | CAGTTTTATCAGTTGCCCACACCTACGAGTTCAAAAAATACTAGGATAGGACCGTTTGCTGATGCAAGTTACAAA |
| | GAGGGAGAAGTTGAACCCGTCGACATGTACACTCCCGATACGGCAGCTGATGAGGAAGCCAGAAAGTTCTGGACT |
| | GAAGATAATAATAATTTATAG |
| Scas1 | ATGTCTGACGCTCCACCACCATTGTCCGAATTGTTCTACAACTCCTCCTACAACCCAGGTTTGTCTATCATTTCT |
| | TACACTTCCATTTACGGTAACGGTACTGAAGTTACCTTTAACGAATTACAATCTATCGTCAACAAGAAGATTACT |
| | GAAGCTATCATGTTCGGTGTCAGATGTGGTGCCGCTATTTTGACTATCATTGTCATGTGGATGATTTCTAAGAAG |
| | AAAAAGACCCCAATTTTCATCATCAACCAAGTTTCTTTATTCTTGATTTTGTTGCACTCCGCTTTCAACTTCAGA |
| | TACTTGTTGTCTAACTACTCTTCCGTCACTTTCGCCTTGACCGGTTTCCCACAATTCATCCACAGAAACGACGTC |
| | CACGTCTACGCTGCTGCTTCTATCTTCCAAGTCTTGTTGGTCGCTTCTATTGAAATTTCCTTAATGTTCCAAATC |
| | AGAGTCATTTTCAAGGGTGATAACTTCAAGAGAATTGGTACTATCTTGACCGCTTTGTCCTCTTCTTTGGGTTTA |
| | GCTACTGTTGCTATGTACTTTGTCACCGCTATTAAGGGTATTATTGCTACCTACAAGGATGTTAACGATACTCAA |
| | CAAAAGTACTTCAACGTTGCTACTATCTTGTTGGCTTCCTCTATCAACTTTATGACCTTGATCTTGGTTATCAAG |
| | TTGATCTTGGCTATCAGATCCAGAAGATTCTTGGGTTTGAAACAATTCGACTCTTTCCATATCTTGTTGATCATG |
| | TCTTTTCAATCTTTGTTGGCCCCATCCATTTTGTTCATTTTGGCTTACTCTTTGGACCCAAACCAAGGTACCGAC |
| | GTCTTGGTTACTGTCGCTACTTTGTTGGTCGTCTTATCTTTGCCATTGTCCTCCATGTGGGCTACTGCTGCTAAC |
| | AACGCCTCCAGACCATCCTCTGTTGGTTCCGACTGGACTCCATCTAACTCCGACTACTACTCTAACGGTCCATCT |
| | TCTGTCAAGACCGAATCTGTCAAATCTGATGAAAAGGTCTCCTTGAGATCCAGAATTTACAACTTGTACCCAAAG |
| | TCTAAGTCTGAATTCGAACAATCCTCCGAACACACTTACGTTGACAAGGTCGACTTGGAAAACAACTTCTACGAA |
| | TTGTCCACCCCAATCACCGAAAGATCTCCATCTTCTATCATTAAGAAGGGTAAGCAAGGTATTTCTACTAGAGAA |
| | ACCGTCAAAAAGTTGGACTCCTTGGATGACATTTACACTCCAAACACTGCTGCTGATGAAGAAGCCAGAAAGTTC |
| | TGGTCTGAAGATGTTTCTAACGAATTGGATTCCTTACAAAAAATCGAAACTGAAACTTCCGATGAATTATCCCCA |
| | GAAATGTTACAATTGATGATTGGTCAAGAAGAAGAAGACGATAACTTATTGGCTACCAAGAAGATCACCGTCAA |
| | GAAGCAA (SEQ ID NO: 197) |
| She | ATGAAACCCGCCGCTGGACCTGCATCTAGTCCATTCGACCCATTTAACCAAACGTTTTACCTGACCGGTCCAGAT |
| | AATACCACTGTACCAGTCTCAGTCCCACAAGTTGACTATATCTGGCATTATATTATTGGAACATCCATCAACTAT |
| | GGTTCTCAGATCGGAGCCTGTTTACTTATGCTTCTTGTGATGTTGACATTGACTTCAAAGTCAAGATTTTCTCGT |
| | GCGGCCACTCTGATTAACGTAGCAAGCTTATTGATTGGAGTAATTCGTTGTGTTCTTTTAGCTGTCTACTTTACT |
| | TCTTCTCTAACTGAATTGTATGCTCTGTTCGTTGGCGATTACAGCCAGGTCCGTAGGTCTGATCTTTGTGTCTCT |
| | GCTGTGGCAACCTTCTTTAGTCTACCACAATTAGTTCTAATAGAAGCTGCTTTGTTTCTACAGGCTTATAGTATG |
| | ATCAAAATGTGGCCATCCCTGTGGAGAGCAGTGGTTTTAGCTATGTCAGTGGTGGTGGCTGTGTGTGCAATCGGT |
| | TTTAAGTTCGCGTCCGTTGTTATGCGTATGAGGTCAACATTAACATTGGACGATTCTTTGGATTTCTGGCTAGTG |
| | GAAGTCGATCTGGCTTTTACAGCAACTACTATTTTTTGGTTTTGTTTCATCTACATTATAAGGTTGGTTATTCAT |
| | ATGTGGGAATATAGAAGCATTTTACCACCAATGGGGTCTGTTTCTGCTATGGAGGTTCTTGTTATGACCAATGGA |
| | GCGTTGATGTTAGTTCCAGTGATTTTCGCCGCAATAGAAATCAATGGTTTATCAAGCTTTGAATCAGGGTCACTG |
| | GTTCATACATCAGTGATTGTATTATTACCTTTAGGTAGCTTGATAGCGCAAGCAATGACACGTCCAGATGGGTAT |
| | GTCCAAAGAACGAATACATCTGGAGCATCAGGCGCAAGTGGTGCACATCCTGGTAGAAATGGATCCGGACACGGT |
| | GGTCATGGTGGTGCGTACTCAAGAGCCATGACTAATACCCTAAATACATTGGATACATTGGATACCGTAGACAGT |
| | AAGACATCCATAATGCATCATCATCATCACCATCATAGAAACCACTCAAATGGCATGAGTAAGACGAAGGCAAAT |
| | AGTGGAACATGGAGCCATGCGTCAGATGCTAACTCCACCAATGCTATGATCAGCGGTGGTATCGCAACTCAAGTT |
| | AGGATTCAAGCTAATCAGTCAACCTTAGGAAATACGGGGATGTCCGGGGGCTCTGGAGCCCCTAATTCTCATACT |
| | CGTAATAACTCATTGGCTGCTATGGAACCAGTGGAGAAGCAACTGCATGATATCGATGCCACACCTTTAAGCGCA |
| | TCTGATTGCAGGGTCTGGGTTGATCGTGAGGTCGAGGTCAGAAGGGACATGGTC (SEQ ID NO: 198) |
| Sj | ATGTACTCCTGGGACGAATTCAGATCCCCAAAGCAAGCTGAAGTTTTGAACCAAACCGTTACCTTGGAAACTATT |
| | GTTTCCACCATTCAATTGCCAATCTCTGAAATTGACTCCATGGAAAGAAACAGATTGTTGACCGGTATGACTGTC |
| | GCTGTTCAAGTTGGTTTAGGTTCCTTCATTTTAGTTTTGATGTGTATTTTCTCTTCCTCTGAAAAGAGAAAGAAG |
| | CCAGTCTTCATCTTCAACTTCGCTGGTAACTTGGTTATGACTTTGAGAGCTATTTTCGAAGTTATCGTTTTGGCT |
| | TCTAACAACTACTCTATCGCTGTTCAATACGGTTTCGCTTTTGCTGCCGTCAGACAATACGTTCACGCCTTCAAC |
| | ATTATCATCTTGTTGTTGGGTCCATTCATCTTGTTCATCGCTGAAATGTCTTTGATGTTGCAAGTTAGAATCATT |
| | TGTTCCCAACACAGACCAACTATGATTACCACCACTGTTATCTCTTGTATTTTCACTGTTGTTACCTTGGCCTTC |
| | TGGATCACCGACATGTCTCAAGAAATTGCTTACCAATTGTTCTTGAAAAACTACAACATGAAGCAAATTGTTGGT |
| | TACTCCTGGTTGTACTTTATCGCTAAGATCACCTTCGCTGCTTCCATTATCTTCCATTCCTCCGTCTTCTCCTTC |
| | AAATTGATGCGTGCTATTTACATTCGTAGAAAGATCGGTCAATTCCCATTCGGTCCAATGCAATGTATCTTCATT |
| | GTTTCCTGTCAATGTTTGATCGTTCCAGCTATTTTCACTTTGATCGATTCTTTCACCCACACTTACGATGGTTTC |
| | TCCTCCATGACTCAATGTTTGTTGATCATCTCCTTACCATTGTCTTCCTTGTGGGCCACCCACACCGCTCAAAAG |
| | TTGCAAACCATGAAGGATAACACTAACCCACCATCTGGTACCCAATTAACCATCAGAGTTGATCGTACTTTCGAC |
| | ATGAAGTTCGTTTCCGACTCCTCTGACGGTTCTTTCACTGAAAAGACCGAAGAAACTTTGCCA |
| | (SEQ ID NO: 199) |
| Sk | ATGTCCGGTAAGCAAGACTTGTCTCCATTAGGTTTGTACTCTTCTTACGACCCTACCAAGGGTTTGATTTCTTAC |
| | ACCTCCTTGTACGGTTCTGGTACTACTGTTACTTTCGAAGAATTGCAAATCTTTGTTAACAAGAAAATTACCCAA |
| | GGTATTTTGTTCGGTACTAGAATCGGTGCCGCCGGTTTAGCTATCATCGTCTTATGGATGGTCTCTAAGAACAGA |
| | AAGACTCCAATTTTCATTATTAACCAAATCTCCTTGTTCTTGATCTTGTTGCACTCCTCTTTGTTCTTGAGATAC |
| | TTGTTGGGTGATTACGCTTCTGTCGTCTTCAACTTTACCTTATTCTCCCAATCCATCTCCAGAAACGATGTCCAC |
| | GTCTACGGTGCCACCAACATGATTCAAGTCTTGTTGGTTGCCGCTGTTGAAATTTCTTTGATTTTTCAAGTCAGA |
| | GTTATTTTCAAAGGTGATTCTTACAAAGGTGTCGGTAGAATCTTGACCTCTATCTCTGCCGTCTTGGGTTTCACT |
| | ACCGTCGTCATGTACTTCATTACTGCCGTTAAGTCCATGACCTCCGTTTACTCTGATTTGACTAAGACTTCCGAC |
| | CGTTACTTCTTTAATATCGCTTCTATTTTATTGTCTTCTTCCGTTAACTTTATGACCTTGTTATTGACCGTCAAG |
| | TTAATTTTGGCCGTCAGATCTCGTAGATTCTTGGGTTTGAAGCAATTCGATTCCTTCCATGTTTTGTTGATTATG |
| | TCCTTCCAAACTTTGATCTTCCCATCTATCTTATTCATCTTGGCTTACGCCTTAAACCCAAACCAAGGTACCGAC |
| | ACTTTAACTTCCATTGCTACCTTGTTAGTCACTTTGTCTTTGCCTTTGTCTTCTATGTGGGCTACCTCTGCTAAC |
| | AACTCCTCCCACCCATCCTCTATCAACACCCAATTCCGTCAAAGAAACTATGACGACGTCTCCTTCAAGACCGGT |
| | ATTACCTCTTTCTACTCCGAATCTTCTAAGCCTTCTTCCAAGTACAGACATACTAACAACTTATATGACTTATAC |
| | CCAGTCTCCCGTACCTCTAACTCCAGATGTAACGGTTACCCAAACGACGGTTCTAAATTAGCTCCAAATCCAAAC |
| | TGTGTTGGTCACAACGGTTCTACTATGTCCGTTAACGACAAGAACGGTGCTCATGCTACCTGTGTTCAAAATAAC |
| | GTCACCTTGAACACCGACTCCACTTTGAACTACTCTAACGTTGACACCCAAGACACTTCCAAGATCTTGATGACC |
| | ACC (SEQ ID NO: 200) |
| Sn | ATGGCTTCTATGGTTCCACCACCAGATTTTGACCCTTACACCCAAGAGTTCATGGTTTTAGGTCCAGATGGTCAA |
| | GAAATCCCAATCTCCATGCAAACCGTCAACGAATACCGTTTGTACACCGCTCGTTTGGGTTTGGCTTATGGTTCC |
| | CAAATTGGTGCCACCTTATTGTTATTGTTGGTTTTGTCTTTGTTAACTAGAAGAGAAAAGAGAAAGTCCGGTATT |
| | TTTATTGTTAACGCTTTGTGTTTGGTTACTAACACCATCAGATGTATTTTGTTGTCCTGCTTTGTCACTTCCACC |
| | TTGTGGCACCCATACACCCAATTCTCTCAAGATACTTCCAGAGTTTCCAAAACTGACGTTAACACCTCTATCGCT |
| | GCCTCTATTTTCACTTTGATTGTCACTGTTTTAATCATGATCTCCTTATCTGTTCAAGTTTGGGTTGTTTGTATT |
| | ACCACTGCTCCATACCAAAGATACATGATTATGGGTGCTACCACCGCTACTGCCATGGTCGCCGTTGGTTACAAG |
| | GCTGCTTTTGTTATCACTTCCATCATTCAAACTTTAAACGGTCAAGACGGTGGTTCCTACTTGGATTTGGTCATG |
| | CAATCTTACATCACTCAAGCTGTCGCTATTTCTTTCTATTCCTGTATTTTCACTTACAAGTTAGGTCACGCTATT |
| | GTTCAAAGAAGAACCTTGAATATGCCACAATTTGGTCCAATGCAAATTATCTTCATCATGGGTTCTTTATTCACT |
| | GGTTTACAATTCGTCAAGAACGTCGATGAATTGGGTATTATCACCCCTACCATTGTTTGTATCTTTTTGCCATTG |
| | TCCGCTATCTGGGCTGGTGTCGTCAACGAAAAGGTTGTCGGTGCTAATGGTCCAGACGCTCATCACAGATTGTTG |
| | CAAGGTGAATTCTACAGAGCTGCTTCTAACTCCACTTACGGTTCTAACTCTTCCGGTACTGTTGTCGACAGATCC |
| | AGACAAATGTCTGTCTGTACTTGTGCTTCTTCTTCCCCATTTGTTAGAAAGAAGTCTGTTGCCGAATGGGACGAT |
| | GAAGCTATTTTAGTTGGTAGAGAATTCGGTTTCTCCCGTGGTGAAGTCGGTGAAAGAGGT |
| | (SEQ ID NO: 201) |
| So | ATGCGTGAACCATGGTGGAAGAACTACTACACCATGAACGGTACCCAAGTCCAAAACCAATCCATCCCAATTTTG |
| | TCCACCCAAGGTTACATTCAAGTTCCATTGTCCACCATCGATAAGGCTGAAAGAAACAGAATTTTGACTGGTATG |
| | ACCGTTTCTGCTCAATTGGCCTTGGGTGTCTTGATCATGGTCATGTCTATTTTGTTGTCCTCCCCAGAAAAGAGA |
| | AAGACCCCAGTTTTCATCGTCAACTCTGCCTCTATCATTTCCATGTGTATTAGAGCTATCTTGATGATTGTCAAC |
| | TTGTGTTCTGAATCCTACTCTTTGGCTGTTATGTACGGTTTCGTCTTCGAATTGGTTGGTCAATACGTTCACGTT |
| | TTTGACATTTTGGTTATGATTATTGGTACCATCATCATTATTACCGCTGAAGTTTCCATGTTGTTGCAAGTCAGA |
| | ATTATTTGTGCTCACGACAGAAAGACTCAAAGAATTGTTACCTGTATCTCTTCTGGTTTATCCTTGATCGTCGTT |
| | GCCTTCTGGTTCACTGATATGTGTCAAGAAATTAAGTACTTGTTGTGGTTGACCCCATACAACAACCACCAAATC |
| | TCTGGTTACTACTGGGTTTACTTCGTCGGTAAGATCTTGTTCGCCGTTTCCATTATGTTCCACTCTGCCGTCTTC |
| | TCCTACAAGTTGTTCCACGCTATCCAAATTAGAAAGAAGATTGGTCAATTCCCATTCGGTCCAATGCAATGTATT |
| | TTAATTATTTCCTGTCAATGTTTGTTCGTTCCAGCTATTTTCACTATCATCGACTCTTTCATCCACACTTACGAC |
| | GGTTTTTCCTCCATGACCCAATGTTTGTTGATCGTCTCTTTGCCATTGTCCTCCTTGTGGGCCTCTTCCACTGCT |
| | TTAAAGTTGCAATCTTTGAAGTCTACCACCTCTCCAGGTGACACTACTCAAGTTTCCATTAGAGTCGACAGAACC |
| | TACGACATCAAGAGAATCCCAACTGAAGAATTGTCTTCTGTTGACGAAACCGAAATCAAGAAGTGGCCA |
| | (SEQ ID NO: 202) |
| Sp | ATGAGACAACCATGGTGGAAAGACTTTACTATTCCCGATGCATCCGCAATTATTCACCAAAATATTACCATTGTC |
| | TCTATTGTAGGAGAGATTGAAGTGCCAGTTTCAACAATTGATGCATATGAAAGAGATAGACTTTTAACTGGAATG |
| | ACTTTGTCTGCCCAACTTGCTTTAGGAGTCCTTACCATTTTGATGGTTTGTCTATTGTCATCATCCGAAAAACGA |
| | AAACACCCAGTTTTTGTTTTTAATTCGGCAAGTATTGTTGCAATGTGTCTTCGGGCCATTTTGAATATAGTGACC |
| | ATATGCAGCAATAGCTACAGTATCCTGGTTAATTACGGGTTTATCTTAAACATGGTTCATATGTATGTCCATGTG |
| | TTTAATATTTTAATTTTGTTGCTTGCACCGGTCATCATTTTTACTGCTGAGATGAGCATGATGATTCAAGTTCGT |
| | ATAATTTGTGCACATGATAGAAAGACACAAAGGATAATGACTGTTATTAGTGCCTGCTTAACTGTTTTGGTTCTC |
| | GCATTTTGGATTACTAACATGTGTCAACAGATTCAGTATCTGTTATGGTTAACTCCACTTAGCAGCAAGACCATT |
| | GTTGGATACTCTTGGCCCTACTTTATTGCTAAAATACTTTTTGCTTTTAGCATTATTTTTCACAGTGGTGTTTTT |
| | TCATACAAACTCTTTCGTGCCATATTAATACGGAAAAAAATTGGGCAATTTCCATTTGGTCCGATGCAGTGTATT |
| | TTAGTTATTAGCTGCCAATGTCTTATTGTTCCAGCTACCTTTACTATAATAGATAGTTTTATCCATACGTATGAT |
| | GGCTTTAGCTCTATGACTCAATGTCTGCTAATCATTTCTCTTCCTCTTTCGAGTTTATGGGCGTCTAGTACAGCT |
| | CTGAAATTGCAAAGCATGAAAACTTCATCTGCGCAAGGAGAAACCACCGAGGTTTCGATTAGAGTTGATAGAACG |
| | TTTGATATCAAACATACTCCCAGTGACGATTATTCGATTTCTGATGAATCTGAAACTAAAAAGTGGACG |
| | (SEQ ID NO: 203) |
| Ss | ATGGATACTAGTATCAATACTCTCAACCCTGCGAATATCATTGTCAACTACACCTTGCCAAATGATCCTAGAGTA |
| | ATTAGTGTCCCATTTGGAGCTTTTGACGAATATGTTAACCAATCTATGCAAAAGGCCATTATCCATGGAGTTTCC |
| | ATTGGTTCATGCACCATAATGCTTTTAATTATTTTGATCTTCAATGTCAAACGCAAGAAGTCGCCAGCTTTCTAT |
| | CTTAATTCGGTTACGTTGACTGCAATGATTATTCGGTCTGCTCTTAATTTGGCATATTTGCTAGGTCCTTTGGCT |
| | GGATTAAGTTTTACGTTCTCCGGCTTGGTAACTCCAGAAACCAATTTCTCTGTCTCTGAAGCCACCAATGCTTTC |
| | CAGGTTATTGTTGTTGCTCTTATCGAGGCGTCCATGACATTTCAGGTGTTCGTCGTCTTCCAATCACCAGAAGTG |
| | AAGAAGTTGGGTATAGCTCTTACCTCCATATCTGCATTCACGGGTGCTGCTGCTGTAGGATTTACTATCAATAGT |
| | ACAATCCAACAATCGAGAATTTATCATTCAGTTGTCAATGGAACTCCTACGCCAACGGTCGCTACCTGGTCTTGG |
| | GTTAGAGATGTGCCTACGATACTTTTTTCTACTTCGGTTAACATAATGTCTTTCATCTTGATTCTCAAGTTAGGG |
| | TTTGCCATAAAGACAAGAAGATACCTTGGCCTTCGGCAATTTGGCAGTTTGCACATCTTATTGATGATGGCTACT |
| | CAAACATTATTGGCCCCATCTATTCTCATTCTTGTACATTACGGATATGGCACATCTCTGAATAGCCAGCTCATT |
| | CTTATAAGTTACTTGCTTGTTGTTTTGTCTTTACCAGTATCCTCTATCTGGGCAGCAACAGCCAACAATTCTCCT |
| | CAACTTCCATCTTCCGCAACTCTTTCATTCATGAACAAAACGACCTCTCACTTTTCTGAAAGC |
| | (SEQ ID NO: 204) |
| Td | ATGTCTGACTCCGCCCAAAACTTGTCCGATTTGGCCTTCAACTCTTCTTATAACCCATTGGACTCCTTTATTACC |
| | TTTACCTCTATCTACGGTGATAACACTGCTGTTAAGTTCTCCGTTTTACAAGACATGGTTGACGTTAATACTAAT |
| | GAAGCCATCGTTTACGGTACCCGTTGTGGTGCTTCTGTCTTGACCCAAATTATCATGTGGATGATTTCTAAAAAC |
| | AGAAGAACCCCAGTCTTTATTATTAACCAAGTTTCTTTGACTTTGATTTTAATTCACTCTGCCTTGTACTTCAAG |
| | TACTTGTTGTCTGGTTTCGGTTCCGTTGTCTACGGTTTGACTGCTTTCCCACAATTGATTAAGCCAGGTGATTTG |
| | AGAGCTTTCGCTGCTGCTAACATCGTTATGGTCTTGTTGGTCGCTTCTATTGAAGCTTCCTTAATCTTCCAAGTC |
| | AAAGTTATCTTCACCGGTGATAACATGAAGAGAGTCGGTTTAATCTTGACTATTATTTGTACTTGTATGGGTTTA |
| | GCTACTGTTACCATGTACTTTATTACTGCCGTCAAGTCTATTGTCTCTTTGTACCGTGACATGTCTGGTTCCTCC |
| | ACCGTTTTATATAACGTTTCTTTAATTATGTTGGCTTCCTCCATCCACTTTATGGCTTTGATCTTGGTTGTCAAA |
| | TTGTTCTTGGCTGTTAGATCTAGAAGATTCTTGGGTTTGAAACAATTCGATTCTTTCCACATTTTGTTGATCATC |
| | TCTTGTCAAACTTTGTTGGTTCCATCTTTATTATTCATTATTGCTTACTCTTTTCCATCTTCTAAGAACATTGAA |
| | TCTTTGAAGGCTATCGCTGTTTTGACCGTCGTTTTGTCTTTGCCATTGTCTTCTATGTGGGCTACTGCTGCTAAT |
| | AACTTCACTAACTCTTCCTCCTCCGGTTCCGACTCCGCTCCAACCAATGGTGGTTTCTACGGTAGAGGTTCTTCC |
| | AACTTGTATCCTGAAAAGACTGATAACAGATCCCCAAAGGGTGCCAGAAACGCTTTATACGAATTAAGATCTAAG |
| | AACAATGCTGAGGGTCAAGCTGATATTTACACCGTTACCGATATTGAAAACGATATTTTCAACGATTTGTCCAAG |
| | CCAGTTGAGCAAAACATTTTCTCTGATGTTCAAATTATTGATTCTCATTCTTTGCATAAGGCTTGTTCTAAAGAA |
| | GACCCAGTCATGACTTTGTACACTCCAAACACTGCTATTGAAGGTGAGGAGAGAAAATTGTGGACTTCTGACTGT |
| | TCCTGTTCCACTAACGGTTCCACCCCAGTTAAGAAGAAGTCCACCGGTGAATACGCCAATTTACCACCACACTTA |
| | TTAAGATATGATGAAAACTACGATGAAGAAGCTGGTGGTAGACGTAAGGCCTCCTTGAAATGG |
| | (SEQ ID NO: 205) |
| Tm | ATGGAGCAAATCCCAGTCTACGAGCGTCCAGGTTTCAACCCACACAAGCAAAACATTACCTTGTTCAAGCATGAT |
| | GGTTCTACTGTTACTGTCGGTTTGCATGAGTTGGACGCCATGTTCACTCATTCCATCAGAGTTGCTGTCGTCTTC |
| | GCCTCTCAAATTGGTGCTTGTGCTTTGTTGTCTGTTATCGTTGCTATGGTCACCAAGAGAGAAAAGAGACGTGCT |
| | TTGTTCTTCTTGCACATTATTTCCTTGTTGTTGGTCGTTGTTCGTTCCGTCTTGCAAATCTTGTACTTCGTCGGT |
| | CCATGGGCTGAAACTTATAATTACGTCGCCTACTACTATGAAGACATTCCTTTGTCTGACAAATTGATTTCCATT |
| | TGGGCTGGTATTATCCAATTGATTTTGAATATCTGTATTTTGTTATCTTTGATCTTGCAAGTTCGTGTCGTTTAC |
| | GCCACCTCTCCAAAATTGAACACTATTATGACTTTAGTCTCTTGTGTTATCGCTTCTATTTCTGTCGGTTTCTTC |
| | TTTACTGTCATCGTTCAAATTTCTGAGGCTATTTTAAACGGTGTTGGTTACGACGGTTGGGTTTACAAAGTCCAT |
| | AGAGGTGTCTTCGCTGGTGCTATCGCCTTCTTCTCTTTCATCTTCATCTTTAAGTTGGCCTTCGCTATCAGAAGA |
| | AGAAAGGCTTTGGGTTTGCAAAGATTCGGTCCATTGCAAGTTATCTTCATCATGGGTTGTCAAACTATGATTGTT |
| | CCAGCTATCTTTGCTACTTTGGAAAACGGTGTTGGTTTCGAAGGTATGTCCTCTTTGACTGCTACCTTGGCTGTC |
| | ATTTCCTTACCATTGTCTTCTATGTGGGCCGCCGCTCAAACCGACGGTCCATCTCCACAATCCACTCCAAGAGAC |
| | GGTTATAGAAGATTCTCTACTCGTAGATCTGCCTTGAACAGATCTGACCCATCTGGTGGTAGATCTGTTGACATG |
| | AACACCTTGGACTCTACCGGTAACGATTCCTTAGCTTTGCACGTTGATAAGACTTTTACTGTTGAATCTTCCCCA |
| | TCCTCCCAATCTCAAGCTGGTCCACACAAGGAAAGAGGTTTCGAATTCGCC (SEQ ID NO: 206) |
| Vp1 | ATGAGTTCCCAATCACACCCACCGCTAATCGATTTATTTTACGATTCCAGTTATGACCCTGGTGAAAGTTTAATT |
| | TATTACACATCCATCTATGGTAATAATACATACATAACTTTTGATGAACTCCAGACGATAGTGAACAAGAAGGTC |
| | ACACAAGGTATCTTATTTGGTGTCAGATGTGGTGCTGCTTTCCTGATGTTGGTAGCAATGTGGTTGATTTCCAAA |
| | AATAAAAGATCTAGAATTTTCATTACCAACCAATGTTGTCTGGTCTTCATGATAATGCATTCTGGTCTTTATTTT |
| | AGGTACCTGCTTTCAAGGTACGGTTCAGTTACTTTCATTCTAACAGGGTTCCAACAACTGCTTACAAGAAATGAC |
| | ATTCATATTTATGGAGCTACTGATTTTATCCAAGTAGCTTTGGTAGCTTGCATAGAATTATCTCTTATTTTCCAA |
| | ATAAAAGTGATATTCGCTGGTACAAACTATGGTAAGTTGGCTAATTATTTCATCACTCTAGGTTCATTATTGGGT |
| | TTAGCCACCTTTGGTATGTACATGCTTACTGCTATTAACGGTACAATAAAATTATACAATAACGAATATGACCCA |
| | AACCAAAGGAAATACTTTAACATTTCTACAATATTGCTTGCATCATCAATTAATATGCTAACGCTGATACTTATA |
| | TTGAAGCTGGTGGCAGCAATTAGAACAAGACGTTACTTAGGTTTGAAGCAATTCGATAGTTTTCACATCCTATTA |
| | ATCATGTCGACTCAAACATTAATAATTCCTTCTATCTTATTTATTCTATCATACAGTTTGAGAGAGGATATGCAT |
| | ACTGATCAATTAATAATCATCGGAAATCTGATCGTGGTATTGTCATTACCATTGTCCTCAATGTGGGCTTCGTCT |
| | CTAAACAATTCAAGTAAACCTACATCTTTGAATACTGATTTCTCAGGGCCAAAATCAAGTGAAGAAGGGACAGCA |
| | ATAAGTTTGCTATCACAAAACATGGAACCATCAATAGTCACTAAATATACAAGAAGATCACCTGGGTTATACCCA |
| | GTAAGCGTGGGTACACCAATTGAAAAAGAAGCATCATACACTCTTTTTGAAGCTACTGACATTGATTTTGAAAGC |
| | AGTAGTAACGATATCACAAGGACTTCA (SEQ ID NO: 207) |
| Vp2 | ATGTCAGGAATTGATGATATGGGTGATAAACCAGATATTTTAGGTTTATTTTATGATGCTAACTATGATCCAGGT |
| | CAAGGTATACTCACATTTATTTCAATGTACGGGAATACTACTATAACTTTTGATGAGTTACAGTTAGAGGTCAAT |
| | AGTTTAATTACAAGTGGTATTATGTTCGGCGTCAGATGTGGTGCTGCTTGTTTGACATTGTTAATAATGTGGATG |
| | ATTTCTAAGAATAAGAAGACTCCAATTTTTATTATTAATCAATGCTCGCTAATCCTTATTATTATGCATTCAGGT |
| | TTATATTTTAAGAATATTCTATCAAATTTGAATTCTTTATCATATATCTTAACTGGGTTTACTCAAAATATCACT |
| | AAAAATAATATACATGTCTTTGGTGCCGCTAATATTATTCAAGTTTTATTAGTAGCAACCATTGAACTGTCGTTA |
| | GTGTTTCAAATTCGAGTCATGTTTAAAGGTGACAGTTTTAGAAAAGCTGGTTACGGTTTGTTGTCAATTGCGTCT |
| | GGTTTGGGTATAGCTACTGTCGTCATGTATTTTTACTCTGCCATTACAAATATGATTGCTGTTTATAATCAAACT |
| | TACAACTCCACTGCTAAATTATTTAACGTTGCAAACATTCTTCTGTCTACATCGATAAATTTTATGACGGTAGTA |
| | TTAATTGTTAAATTATTTTTGGCTGTTAGATCAAGAAGATATTTGGGTTTAAAGCAGTTCGATAGTTTCCATATT |
| | TTATTGATTATGTCATGTCAAACATTGATTGTACCATCAATTCTTTTTATCTTATCATACGCTTTAAGTACTAAG |
| | CTGTACACTGATCATTTAGTTGTCATTGCAACTTTATTAGTCGTTCTATCTTTACCATTATCTTCGATGTGGGCA |
| | AGCGCTGCAAATAATTCTCCTAAACCAAGCTCGTTTACAACCGATTATTCAAACAAGAATCCTAGTGACACACCA |
| | AGCTTCTACAGTCAAAGTATTAGTTCCTCGATGAAAAGCAAATTCCCAAGCAAATTCATACCCTTCAATTTCAAG |
| | TCTAAAGACAATTCTTCTGACACTAGATCAGAAAATACATATATTGGCAATTATGACATGGAAAAGAATGGATCA |
| | CCAAATCACTCTTATTCTTCCAAAGATCAAAGTGAAGTTTACACTATAGGTGTAAGCTCTATGCACACAGATATA |
| | AAGTCACAAAAGAATATCAGTGGACAGCATTTATATACCCCAAGTACAGAGATTGATGAAGAAGCTAGAGACTTC |
| | TGGGCGGGCAGAGCTGTTAATAATTCAGTTCCAAATGACTATCAACCATCTGAGTTACCAGCATCGATTCTTGAA |
| | GAATTGAATTCACTGGATGAAAATAATGAAGGTTTCTTGGAGACAAAAAGAATAACATTTAGAAAACAA |
| | (SEQ ID NO: 208) |
| Y1 | ATGCAATTGCCACCACGTCCAGACTTCGACATTGCCACTTTGGTTGCCTCTATCACTGTTCCAGAAACTGAATTG |
| | GTCTTGGGTCAAATGCCATTGGGTGCTTTAGAACAATTGTACCAAAACAGATTGCGTTTGGCTATTTTGTTCGGT |
| | GTCAGAGTCGGTGCTGCTGTTTTGACCTTGATTGCTATGCACTTAATCTCCAAGAAGAACAGAACCAAGATCTTG |
| | TTCTTGGCTAACCAAATGTCTTTGATCATGTTGATCATCCATGCTGCTTTGTACTTCAGATTCTTGTTGGGTCCA |
| | TTCGCCTCCATGTTGATGATGGTTGCTTACATCGTTGATCCAAGATCTAACGTCTCTAACGATATCTCTGTTTCT |
| | GTTGCCACCAACGTTTTCATGATGTTGATGATTATGTCCGTCCAATTGTCTTTGGCTGTTCAAACCCGTTCTGTT |
| | TTCCACGCTTGGTTGAAGTCTCGTATTTACGTTACCGTTGGTTTAATCTTGTTGTCCTTGGTCGTCTTCGTCTTC |
| | TGGACCACCCACACTATCGTTTCTTGTATCGTTTTAACCCATCCAACTAGAGACTTGCCATCTATGGGTTGGACT |
| | AGATTAGCTTCTGACGTTTCCTTCGCTTGTTCTATCTCTTTCGCTTCTTTGGTCTTGTTGGCTAAGTTGGTCACC |
| | GCCATCAGAGTTAGAAAGACCTTGGGTAAGAAGCCATTGGGTTACACCAAGGTTTTGGTCATCATGTCCACTCAA |
| | TCTTTAGTCGTTCCATCTATCTTGATTATCGTTAACTACGCTTTGCCAGAAAAAAACTCTTGGATCTTGTCTGGT |
| | GTCGCTTACTTGATGGTTGTTTTGTCCTTACCATTGTCCTCCATTTGGGCTACCGCCGTCCATGACGACGAAATG |
| | CAATCCAACTACTTGTTGTCTGCCTTGAAAGATGGTCACGTTCAACCATCCGAATCTAAGTTGAAGACTGTTTTC |
| | TTGAACAGATTGAGACCATTCTCTACTACCACTAACAGAGACGATGAATCCTCTGTTGATTCCCCAGCCATGCCA |
| | TCTCCAGAATCTGATGTTACCTTCTTGAACACTGGTTTCGAATGTGACGAAAAGATG (SEQ ID NO: 209) |
| Zb | Sequence reported10 |
| | ATGTCTGGTTTGGCTAACAACACCTCTTACAACCCATTGGAATCTTTCATTATTTTCACTTCTGTTTACGGTGGT |
| | GATACCATGGTTAAGTTCGAAGACTTGCAATTAGTCTTCACCAAGCGTATTACTGAAGGTATTTTGTTCGGTGTC |
| | AAGGTTGGTGCCGCTTCTTTGACTATGATTGTTATGTGGATGATTTCCAGAAGAAGAACCTCCCCAATCTTCATC |
| | ATGAACCAATTGTCTTTGGTTTTCACCATCTTGCACGCTTCTTTTTACTTTAAGTACTTATTGGACGGTTTCGGT |
| | TCTATTGTCTACACTTTGACCTTGTTCCCACAATTAATTACTTCCTCTGACTTGCACGTTTTCGCTACTGCTAAC |
| | GTTGTTGAAGTCTTATTGGTTTCTTCCATCGAAGCCTCTTTGGTTTTCCAAGTCAACGTCATGTTCGCTGGTTCT |
| | AACCACAGAAAGTTCGCTTGGTTGTTGGTCGGTTTCTCTTTGGGTTTGGCTTTGGCCACTGTCGCTTTGTACTTC |
| | GTTACTGCTGTCAAGATGATCGCTTCCGCTTACGCTTCTCAACCACCAACTAACCCAATCTACTTCAACGTTTCC |
| | TTGTTCTTGTTGGCTGCCTCCGTTTTCTTGATGACTTTAATGTTGACCGTCAAGTTGATCTTGGCTATCAGATCC |
| | AGAAGATTCTTGGGTTTGAAGCAATTCGACTCTTTCCACATTTTGTTGATTATGTCTTGTCAAACTTTGATCGCT |
| | CCATCTGTTTTGTACATCTTGGGTTTTATTTTGGATCACAGAAAGGGTAACGACTACTTGATTACCGTCGCTCAA |
| | TTGTTGGTCGTTTTGTCTTTGCCATTGTCCTCCATGTGGGCCACTACTGCTAACGATGCTTCCTCCGGTACTTCT |
| | ATGTCTTCCAAGGAATCCGTCTACGGTTCTGATTCCTTATACTCTAAGTCTAAGTGTTCCCAATTCACCAGAACC |
| | TTCATGAACAGATTCTCTACTAAGCCAACTAAGAACGACGAAATTTCTGATTCCGCTTTCGTCGCTGTTGATTCC |
| | TTGGAAAAGAACGCTCCACAAGGTATCTCTGAACACGTTTGTGAATTCCCACAATCTGACTTATCTGATCAAGCT |
| | ACTTCCATCTCCTCCAGAAAAAAGGAAGCTGTTGTTTACGCTTCCACTGTTGATGAAGATAAGGGTTCTTTCTCC |
| | TCTGACATCAACGGTTACACTGTTACCAACATGCCATTGGCTTCCGCTGCTTCTGCTAACTGTGAAAACTCCCCA |
| | TGTCACGTTCCAAGACCATACGAAGAAAACGAAGGTGTCGTCGAAACCAGAAAAATTATTTTGAAGAAGAACGTC |
| | AAATGGTAG (SEQ ID NO: 210) |
| Zr | Sequence reported10 (SEQ ID NO: 211) |
| | ATGAGTGAGATTAACAATTCTACCTACAATCCAATGAATGCATATGTAACGTTTACATCAATATATGGTGATGAT |
| | ACTATGGTACGTTTCAAAGATGTGGAATTGGTAGTTAACAAAAGGGTTACAGAAGCCATTATGTTCGGCGTCAAA |
| | GTTGGTGCAGCTTCGTTGACACTCATCATCATGTGGATGATCTCTAAGAAAAGAACAACACCGATATTTATCATA |
| | AATCAGTCTTCGCTTGTATTTACCATAATACATGCTTCGCTTTATTTTGGGTACCTTTTGTCAGGATTTGGTAGT |
| | ATAGTTTACAATATGACATCGTTCCCGCAGTTAATAAGCTCCAATGACGTTCGTGTGTACGCAGCTACAAATATT |
| | TTTGAGGTCCTGTTGGTAGCATCTATCGAAATCTCTCTGGTTTTTCAGGTCAAAGTTATGTTTGCCAACAATAAT |
| | GGTCGAAGATGGACTTGGTGTTTGATGGTAGTTTCCATAGGGATGGCACTAGCTACTGTAGGACTTTATTTTGCC |
| | ACTGCCGTTGAGTTGATCAGAGCTGCTTACAGCAATGATACTGTTAGCCGCCATGTTTTTTACAATGTTTCTCTG |
| | ATCTTACTAGCGTCATCTGTCAATCTAATGACACTAATGCTAGTGGTAAAATTAGTATTAGCGATCAGATCAAGA |
| | AGATTTTTGGGGTTAAAACAGTTTGACAGTTTCCACATATTACTTATAATGTCTTGCCAGACTCTAATAGCACCT |
| | TCCATTCTATTCATTTTGGGTTGGACCTTAGACCCTCATACTGGTAATGAGGTTTTAATTACAGTTGGTCAATTG |
| | CTAATAGTACTGTCATTACCGCTGTCATCTATGTGGGCTACAACCGCTAACAATACCAGTTCATCTAGTAGTTCG |
| | GTGTCCTGTAATGACAGCTCTTTTGGTAATGACAATCTCTGTTCCAAGAGTTCGCAATTTAGAAGAACTTTTATG |
| | AATAGATTCCGTCCCAAGTCGGTTAATGGTGACGGTAATTCTGAAAATACCTTTGTTACAATTGATGATTTGGAA |
| | AAAAGCGTTTTTCAAGAATTATCAACACCTGTTAGCGGAGAATCAAAGATAGATCATGATCATGCAAGTAGTATT |
| | TCATGTCAAAAGACATGTAATCATGTTCATGCTTCGACAGTGAATTCAGATAAGGGATCTTGGTCCTCTGATGGT |
| | AGTTGTGGCAGTTCTCCGTTAAGAAAGACTTCCACCGTTAATTCTGAAGATTTACCTCCACATATATTGAGCGCC |
| | TACGATGACGATCGAGGTATAGTAGAAAGTAAAAAAATTATCCTAAAGAAATTATAG |

Construction of peptide secretion vectors. The peptide secretion vector is based on pRS423 (HIS3 selection marker, 2μ origin of replication)⁵⁸. The peptide coding sequence was designed based on the natural S. cerevisiae α-factor precursor, similar as described previously⁴⁷. In brief: To make a general secretion cassette the MFα1 gene was amplified with or without the Ste13 processing site (EAEA). The actual sequences for the peptide ligands were inserted via a unique restriction site (AflII) after the pre- and pro-sequence, thus the peptide DNA sequence can be swapped by Gibson assembly⁶⁷ using peptide-encoding oligos codon-optimized for expression in yeast. The DNA and resulting protein sequences of all peptide precursor genes are listed in Table 7. The constitutive ADH1 promoter or the ligand-dependent FUS1 and FIG1 promoters were used to drive peptide expression. Promoters were amplified from S. cerevisiae genomic DNA.

```
TABLE 7
DNA sequences of peptide ligand expression cassettes:
Peptide expression cassettes were cloned into vector pRS423 under control of the
constitutive ADH1 promoter or the peptide inducible FUS1p promoter. The first
row shows the amino acid sequence of the designed generic peptide ligand precursor.
The second row shows its DNA sequence. This precursor was used to clone in all other
peptide ligand sequences. The sequences were ordered as oligonucleotides codon-optimized
  for expression in yeast and inserted into the cassette by Gibson assembly
  (Gibson et al., Nat. Methods 2009). The secretion signal is highlighted in green,
the Kex2 processing site is marked in bold grey, the Ste13 processing
  site encoding sequence is marked in bold. Peptide sequences are
ordered alphabetically according to their 2-letter species code.
Amino acid sequence of peptide precursors
RFPSIFTAVLFAASSALAAPVNTTTEDETAQIPAEAVIGYLDLEGDFDVAVLPFSNSTNNGLLFINTTIASIAAKEEGVSLDKR(EAEA)-
(SEQ ID NO: 212) followed by peptide sequence-TAG
DNA sequence of peptide pre-pro precursor
Without Ste13 processing site (EAEA)
AGATTTCCTTCAATTTTTACTGCAGTTTTATTCGCAGCATCCTCCGCATTAGCTGCTCCAGTCAACACTACAACAGAAGATGAAACGGCAC
AAATTCCGGCTGAAGCTGTCATCGGTTACTTAGATTTAGAAGGGGATTTCGATGTTGCTGTTTTGCCATTTTCCAACAGCACAAATAACGG
GTTATTGTTTATAAATACTACTATTGCCAGCATTGCTGCTAAAGAAGAAGGGGTATCTTTGGATAAAAGA-(SEQ ID NO: 213)
followed by peptide sequence-TAG
Plus Ste13 processing site
AGATTTCCTTCAATTTTTACTGCAGTTTTATTCGCAGCATCCTCCGCATTAGCTGCTCCAGTCAACACTACAACAGAAGATGAAACGGCAC
AAATTCCGGCTGAAGCTGTCATCGGTTACTTAGATTTAGAAGGGGATTTCGATGTTGCTGTTTTGCCATTTTCCAACAGCACAAATAACGG
GTTATTGTTTATAAATACTACTATTGCCAGCATTGCTGCTAAAGAAGAAGGGGTATCTTTGGATAAAAGAGAGGCTGAAGCT-
(SEQ ID NO: 214) followed by peptide sequence-TAG
```

| Code | DNA sequence |
|---|---|
| Bb | ggtgtatgagaccaggtcaaccatgttgg (SEQ ID NO: 215) |
| Bc | tggtgtggtagaccaggtcaaccatgt (SEQ ID NO: 216) |
| Ca | ggtttcagattgaccaacttcggttacttcgaaccaggt (SEQ ID NO: 217) |
| Cgu | aagaagaactctagattcttgacctactggttcttccaaccaatcatg (SEQ ID NO: 218) |
| Cl | aagtggaagtggatcaagttcagaaacaccgacgttatcggtTAG (SEQ ID NO: 219) |
| Gc | ggtgactggggttggttctggtacgttccaagaccaggtgacccagctatg (SEQ ID NO: 220) |
| Hj | tggtgttacagaatcggtgaaccatgttgg (SEQ ID NO: 221) |
| Kp | cagatggagaaacaacgaaaagaaccaaccattcggt (SEQ ID NO: 222) |
| Le | ggatgtggaccagatacggtagattctctccagtt (SEQ ID NO: 223) |
| Pb | ggtgtaccagaccaggtcaaggttgt (SEQ ID NO: 224) |
| Pd | ttctgttggagaccaggtcaaccatgtggt (SEQ ID NO: 225) |
| Sc | tggcactggttgcaattgaagccaggtcaaccaatgtac (SEQ ID NO: 226) |
| Sj | gtttctgacagagttaagcaaatgttgtctcactggtggaacttcagaaacccagacaccgctaacttg (SEQ ID NO: 227) |
| So | acctacgaagacttcttgagagtnacaagaactggtggtctttccaaaacccagacagaccagacttg (SEQ ID NO: 228) |
| Vp | tggcactggttggaattggacaacggtcaaccaatctac (SEQ ID NO: 229) |
| Zr | cacttcatcgaattggacccaggtcaaccaatgttc (SEQ ID NO: 230) |

CRISPR-Cas9 system. The Cas9 expression plasmid was constructed by amplifying the Cas9 gene with TEF1 promoter and CYC1 terminator from p414-TEF1p-Cas9-CYC1t⁵⁹ cloned into pAV115⁶⁸ using Gibson assembly⁶⁷. For short genes, MFALPHA1/2 and MFA1/2, a single gRNA was cloned into a gRNA acceptor vector (pNA304) engineered from p426-SNR52p-gRNA.CAN1.Y-SUP4t⁶⁹ to substitute the existing CAN1 gRNA with a NotI restriction site. gRNAs were cloned into the NotI sites using Gibson assembly⁶⁷. Double gRNAs acceptor vector (pNA0308) engineered from pNA304 cloned with the gRNA expression cassette from pRPR1gRNAhandleRPR1t⁷⁰ with a HindIII site for gRNA integration. gRNAs were cloned into the NotI and HindIII sites using Gibson assembly⁶⁷. For engineering yeast using the Cas9 system, cells were first transformed with the Cas9 expressing plasmid. Following a co-transformation of the gRNA carrying plasmid and a donor fragment. Clones were then verified using colony PCR with appropriate primers.

Construction of core peptide/GPCR language S. cerevisiae acceptor strains. Core S. cerevisiae strains yNA899 and yNA903 are derivatives of strain BY4741 (MATa leu2Δ0 met15Δ0 ura3Δ0 his3Δ1) and BY4742 (MATα lys2Δ0 leu2Δ0 ura3Δ0 his3Δ1), respectively. They are deleted for both S. cerevisiae mating GPCR genes (stet and ste3) and all mating pheromone-encoding genes (mfa1, mfa2, mfa1, mfa2) as well as for the genes far1, sst2 and bar1. All genes were deleted as clean open reading frame-deletions using CRISPR/Cas9 as described below. In most cases, except for MFA genes, two gRNAs were designed for each gene to target sequences on the 5′ and 3′ end of the gene's open reading frame (all gRNA sequences are listed in Table 8). Genes were deleted sequentially. After each round of gene deletion, strains were cured from the gRNA vector and directly used for deleting the next gene.

TABLE 8
gRNAs used for genome engineering:

| Target gene | | |
|---|---|---|
| or locus | 5′ gRNA | 3′ gRNA |
| STE2 | CAGAATCAAAAATGTCTGATG | ATGAGGAAGCCAGAAAGTT |
| | (SEQ ID NO: 231) | (SEQ ID NO: 232) |
| STE3 | CATACAAGTCAGCAATAATA | ATAGTTCAGAAAATACTGC |
| | (SEQ ID NO: 233) | (SEQ ID NO: 234) |
| MFalpha1 | AAAACTGCAGTAAAAATTGA | ATTGGTTGCAGTTAAAACC |
| | (SEQ ID NO: 235) | (SEQ ID NO: 236) |
| MFalpha2 | CGCTAAAATAAAAGTGAGAA | ACTGGTTGCAACTCAAGCC |
| | (SEQ ID NO: 237) | (SEQ ID NO: 238) |
| MFa1 | AAAGACCAGCAGTGAAAAGA | |
| | (SEQ ID NO: 239) | |
| MFa2 | TTCCACACAAGCCACTCAGA | |
| | (SEQ ID NO: 240) | |
| FAR1 | AAAATACACACTCCACCAAG | GCAAAGAATTCATCAGACCC |
| | (SEQ ID NO: 241) | (SEQ ID NO: 242) |
| BAR1 | TCTTTGTTTGAAACTTATTT | TTGTACATGAAACTAAATAT |
| | (SEQ ID NO: 243) | (SEQ ID NO: 244) |
| SST2 | GTAAGATGGTGGATAAAAAT | CATCTTTGTATACGTCTGAC |
| | (SEQ ID NO: 245) | (SEQ ID NO: 246) |
| STE12 | AATAACCAATAGTAGAACAG | CTGTTCTACTATTGGTTATT |
| | (SEQ ID NO: 247) | (SEQ ID NO: 248) |
| ΔSTE2 (insertion | ATATTCAAGATTTTTTTCTG | |
| of TDH3p-xySte2) | (SEQ ID NO: 249) | |
| ΔSTE3 (insertion | ATGTGTAAATGAAGGAATAA | |
| of TDH3p-xySte2) | (SEQ ID NO: 250) | |
| STE12 (replacement | TGAAGTCAGTAAAGCTACTC | |
| by Ste12*) | (SEQ ID NO: 251) | |
| SEC4 (replacement | TCCTCGTGGGCCAGGACTAG | |
| of SEC4 promoter | (SEQ ID NO: 252) | |
| by OSRs) | | |
| SEC4 (replacement | CATTCTACCTCTAGGGAAGC | |
| of SEC4 promoter | (SEQ ID NO: 253) | |
| by CYCt-OSRs) | | |

Genomic integration of color read-outs and GPCR genes. yNA899 was used to insert a FUS1 and a FIG1 promoter-driven yeast codon-optimized RFP (coRFP) into the HO locus. Using yeast Golden Gate (yGG) a transcription unit of the appropriate promoter (FUS1 or FIG1) was assembled with coRFP coding sequence and a CYC1 terminator into pAV10.HO5.loxP. Following yGG assembly and sequence verification, plasmid was digested with NotI restriction enzyme and transformed into yeast cells. Clones are then verified using colony PCR with appropriate primers. The resulting strain JTy014 was used for all GPCR characterizations by transforming it with the appropriate GPCR expression plasmids. GPCR genes were integrated into the ΔSte2 locus of yNA899. The GPDp-xySte2-Ste2t expression cassette for Bc.Ste2, Sc.Ste2 and Ca.Ste2 was used as repair fragment. The resulting generic locus sequence is listed in Table 5.

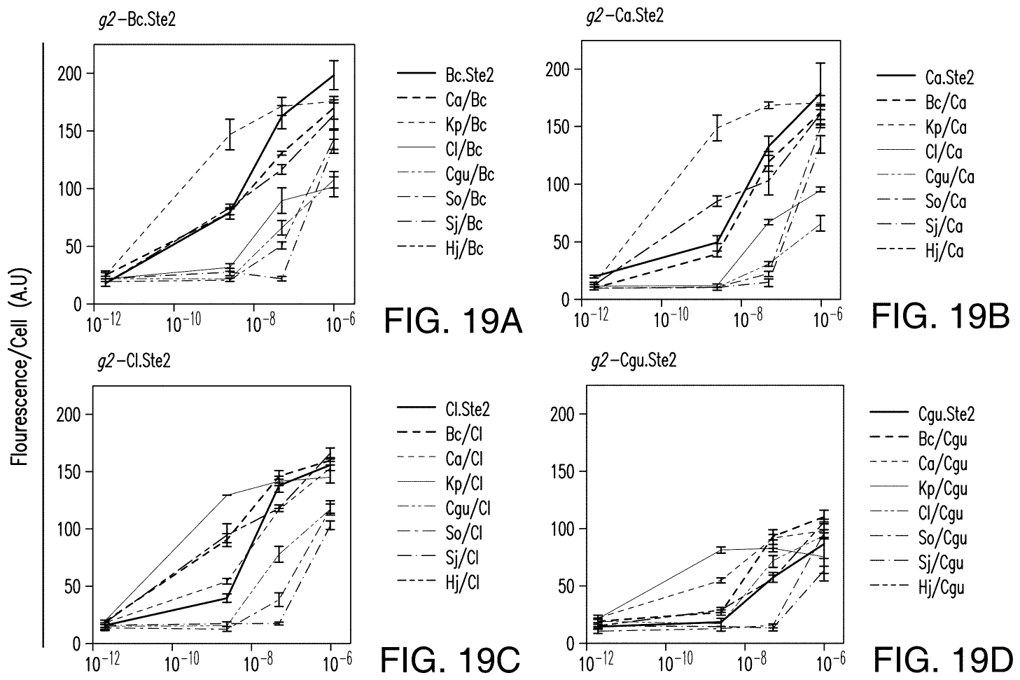

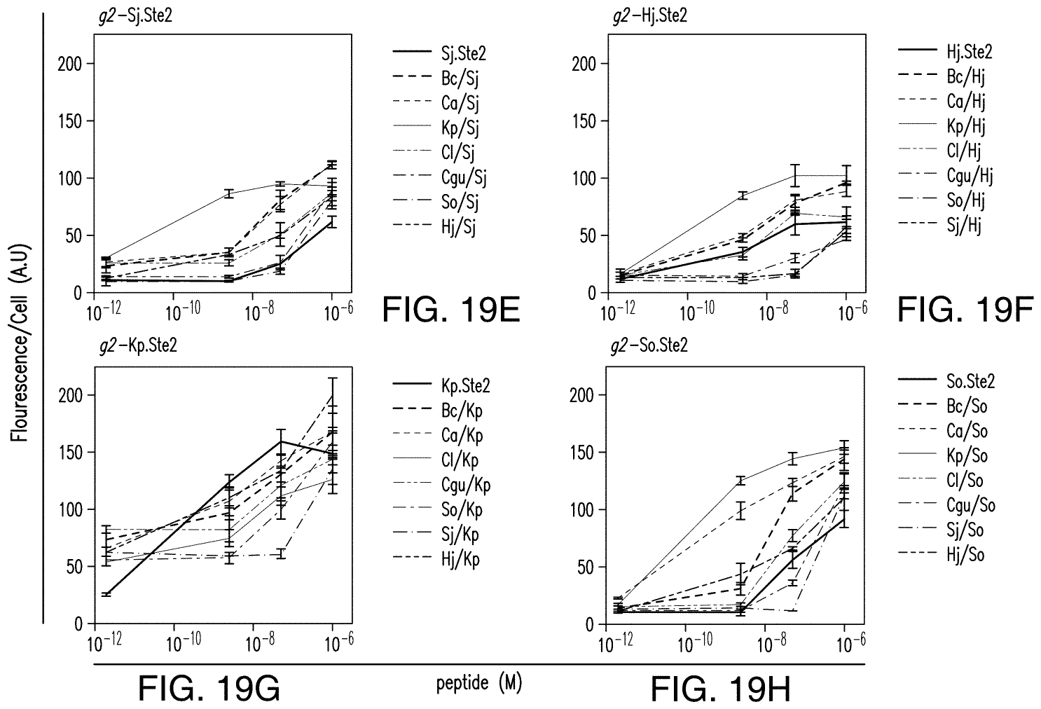

Construction of peptide-dependent yeast strains. yNA899 was used as parent. First, expression cassettes for Bc.Ste2 and Ca.Ste2 were integrated into the ΔSte2 locus as described above. The DNA binding domain of the pheromone-inducible transcription factor Ste12 (residues 1-215) was then replaced with the zinc-finger-based DNA binding domain 43-8⁷¹ (the resulting Ste12 variant is referred to as orthogonal Ste12*, FIG. 19). The natural SEC4 promoter was then replaced with differently designed synthetic orthogonal Ste12* responsive promoters (OSR promoters) and resulting strains were screened for best performers (with regard to peptide-dependent growth). Resulting strains ySB270 (Ca.Ste2) and ySB188 (Vp1.Ste2) feature OSR4, strain ySB265 (Bc.Ste2) features OSR1. Genomic engineering was achieved using CRISPR-Cas9 and the guide RNAs listed in Table 8.

GPCR on-off activity and dose response assay. GPCR activity and response to increasing dosage of synthetic peptide ligand was measured in strain JTy014 using the genomically integrated FUS1-promoter controlled coRFP as a fluorescent reporter. JTy014 strains carrying the appropriate GPCR expression plasmid were assayed in 96-well microtiter plates using 200 μl total volume, cultured at 30° C. and 800 RPM. Cells were seeded at an A₆₀₀ of 0.3 (Note: all herein reported cell density values are based on A₆₀₀ measurements in 96-well plates of a 200 μl volume of cultures with a path length of ˜0.3 cm performed in an Infinite M200 plate reader from Tecan) in SC media lacking uracil (selective component). All measurements were performed in triplicates. RFP fluorescence (excitation: 588 nm, emission: 620 nm) and culture turbidity (A₆₀₀) were measured after 8 hours using an Infinite M200 plate reader (Tecan). Since the optical density values were outside the linear range of the photodetector, all optical density values were first corrected using the following formula to give true optical density values:

$${A}_{true} = \frac{k \cdot {A}_{meas}}{{A}_{sat} - {A}_{meas}} \qquad ( Eq . 1 )$$

where Aₘₑₐₛ is the measured optical density, Aₛₐₜ is the saturation value of the photodetector and k is the true optical density at which the detector reaches half saturation of the measured optical density³⁶. Dose-response was measured at different concentrations (11 five-fold dilutions in H₂O starting at 40 μM peptide, H₂O was used as “no peptide” control) of the appropriate synthetic peptide ligand. All fluorescence values were normalized by the A₆₀₀, and plotted against the log(10)-converted peptide concentrations. Data were fit to a four-parameter non-linear regression model using Prism (GraphPad) in order to extract GPCR-specific values for basal activation, maximal activation, EC₅₀ and the Hill coefficient. Fold-activation was calculated for each GPCR as the maximum A₆₀₀-normalized fluorescence of peptide-treated cells divided by the A₆₀₀ normalized fluorescence value of water-treated cells.

GPCR orthogonality assay using synthetic peptides. GPCR activation was individually measured in 96-well microtiter plates in triplicate using each of the synthetic peptides (10 μM). Cells were seeded at an A₆₀₀ of 0.3 in 200 μl total volume in 96-well microtiter plates, cultured at 30° C. and 800 rpm. Endpoint measurements were taken after 12 hours, as described above. Percent receptor activation was calculated by setting the A₆₀₀-normalized fluorescence value of the maximum activation of each GPCR (not necessarily its cognate ligand) to 100% and the value of water treated-cells to 0%, with any negative values set to 0%).

Peptide secretion fluorescent halo assay. JTy014 was transformed with the appropriate GPCR expression plasmid and resulting strains were used as sensing strains. yNA899 was transformed with the appropriate peptide secretion plasmids and used as secreting strains. Sensing strains for all 16 peptides were individually spread on SC plates. Briefly, 0.5% agar was melted and cooled down to 48° C., cells are added to an aliquot of agar in a 1:40 ratio (100 μL of cells into 4 mL of agar for a 100 mm petri dish and 200 μL of cells into 8 mL of agar for a Nunc Omnitray), mixed well and poured on top of a plate containing solidified medium. A 10 μL dot of each of the secreting strains was spotted on each of the sensing strain plates. Plates were incubated at 30° C. for 24-48 h and imaged using a BioRad Chemidoc instrument and proper setting to visualized RFP signal (light source: Green Epi illumination and 695/55 filter).

Peptide secretion liquid culture assay. Peptide secretion in liquid culture was examined by co-culturing a secretion and a sensing strain (expressing the cognate GPCR) and measuring fluorescence of the induced sensing strain. Peptide secretion was under control of the constitutive ADH1 promoter. Secretion strains for each peptide were constructed by transforming yNA899 with the appropriate peptide expression construct (pRS423-ADH1p-xy.Peptide) along with an empty pRS416 plasmid. Sensor strains were constructed by transforming JTy014 with the appropriate GPCR expression construct (pRS416-GPD1p-xy.Ste2) along with an empty pRS423 plasmid. Matching the auxotrophic markers of the secretion and sensor strains allowed for robust co-culturing. Secreting and sensing strains were seeded in a 1:1 ratio each at an A₆₀₀ of 0.15, and A₆₀₀ and red fluorescence were measured after 12 hours. Experiments were run in triplicate. An unpaired t-test was performed for each peptide with an alpha value=0.05 to determine if differences in secretion between constructs containing or not containing the Ste13 processing site were significant. A single asterisk indicates a P value <0.05; a double asterisk indicates a P value <0.01.

Secretion orthogonality assay. The same sensing and secretion strains as described for the “Peptide secretion liquid culture assay” (above) were used to confirm orthogonality of secreted peptide in co-culture. Only the constructs that retained the Ste13 processing site were used. To determine orthogonality, each of the 16 constructed secretion strains were co-cultured 1:1 each at an A₆₀₀ of 0.15 with the corresponding sensor strains to test for GPCR activation by non-cognate peptide, and A₆₀₀ and red fluorescence were measured after 14 hours. Experiments were run in triplicate. Percent activation of the sensor strain was normalized by setting the maximum observed activation of the sensor strain (not necessarily by the cognate ligand) to 100%, and setting the basal fluorescence from co-culturing each sensor strain with a non-secreting strain to 0% activation, with any negative values set to 0%.

Transfer functions through minimal communication units. yNA899 with the appropriate GPCR integrated into the Ste2 locus using the CRISPR system described above were transformed with the appropriate peptide secretion plasmid (pRS423-FIG1p-xy.Peptide retaining the Ste3 processing site) and resulting strains were used as cell 1 (c1, sender). JTy014 was transformed with the appropriate GPCR expression plasmid (pRS416-GPD1p-xy.Ste2) and used as cell 2 (c2, reporter). As c1 and c2 didn't have the same auxotrophic markers, validated strains were grown overnight in selective media and then seeded at a 1:1 ratio each at an A₆₀₀ of 0.15 in SC media. Cells were cultured in a total volume of 200 μl in 96-well microtiter plates and c1 was induced with the appropriate synthetic peptide at 2.5 nM, 50 nM, and 1000 nM, using water as the 0 nM control. Red fluorescence and A₆₀₀ were measured after 12 hours. As a control, c2 was co-cultured with a non-secreting strain carrying an empty pRS423 plasmid and induced with the appropriate synthetic peptide at the concentrations listed above.

Multi-yeast paracrine ring assay. Communication loops were designed so that a single fluorescent measurement would indicate signal propagation through the full ring topology. An initiator strain was constructed by integrating the Ca.Ste2 into JTy014 and transforming it with a constitutive Kp peptide secretion plasmid (pRS423-ADH1p-Kp.Peptide). Linker strains from the transfer functions experiment (without a fluorescent readout) were used to complete each communication ring. Communication rings were seeded in triplicate at equal ratios (A₆₀₀=0.02 each) in 10 mL selective 2×SC-His medium and incubated at 30° C. with 250 RPM shaking for 36 hours. 200 μL samples were taken for a fluorescent measurement of red fluorescence (588 nm/620 nm excitation/emission) in technical triplicate in a 96-well black clear-bottom plate and normalized by A₆₀₀. To demonstrate that communication is contingent on a complete ring topology, a control with the first linker yeast strain in each ring dropped out was performed in parallel. The panels compare the normalized red fluorescent signal for each ring to the dropout control, with the fold change induction of the completed ring indicated.

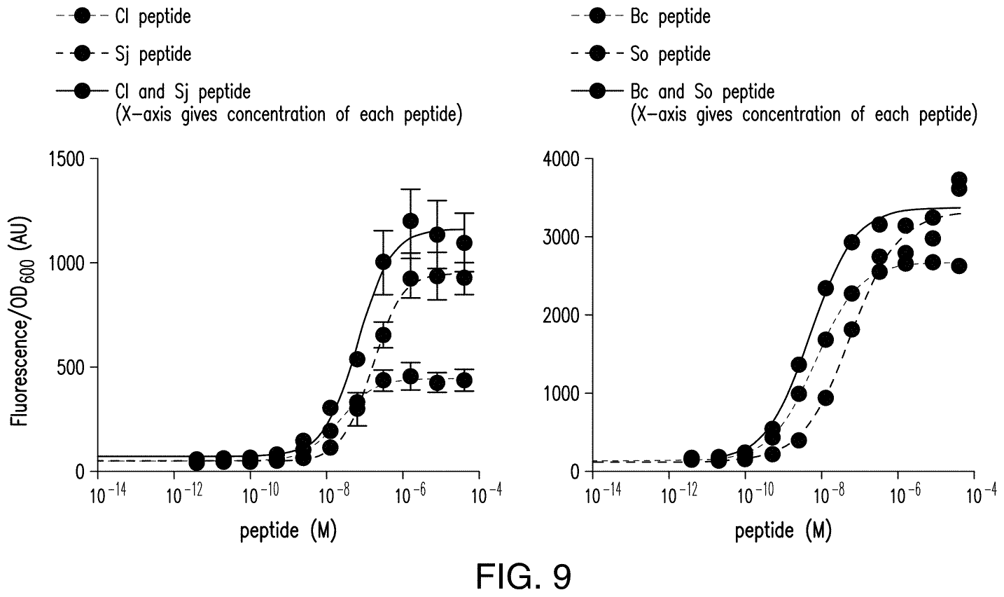

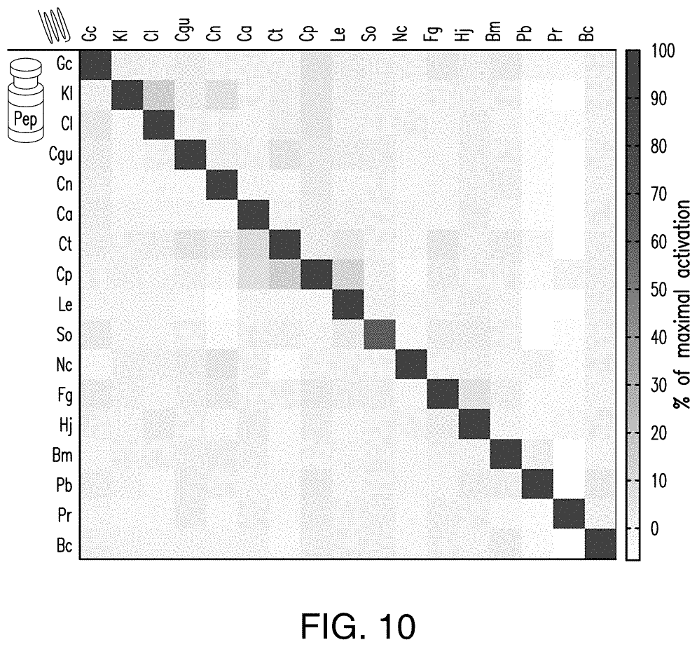

Tree topology assay. Bus and tree topologies were designed so that a single fluorescent measurement would indicate signal propagation through the full topology. To enable branched topologies with two-input nodes, an additional orthogonal GPCR was integrated into the STE3 locus using the CRISPR-Cas9 system described above (strains ySB315 and ySB316, Table 2). Single and dual dose-response characteristics of ySB315 and ySB316 confirmed the ability to activate either or both co-expressed GPCRs (FIG. 9). ySB315 and ySB316 were then transformed with the appropriate peptide secretion plasmids and combined with linker strains validated from the transfer functions experiment and ySB98 transformed with an empty pRS423 plasmid as a fluorescent readout of communication. Communication topologies were seeded at equal ratios (A₆₀₀=0.02 each) in 10 mL selective 2×SC-His medium and incubated at 30° C. with 250 RPM shaking for 16 hours. 200 μL samples were taken for a fluorescent measurement of red fluorescence (588 nm/620 nm excitation/emission) in technical triplicate in a 96-well black clear-bottom plate and normalized by A₆₀₀. To demonstrate that dual-input nodes can be activated by either one or two input peptides, different combinations of the input peptides were added at 1 uM each (see FIG. 26 for key to FIG. 18E-F). Fold change compared to no added peptide is indicated.

Flow cytometry. Cells were seeded at an A₆₀₀ of 0.3. Cells were exposed to the indicated peptide concentrations and cultured for 12 h in 96-well microtiter plates in a total volume of 200 μl at 30° C. and 800 RPM shaking. For each sample 50,000 cells were analyzed using a BD LSRII flow cytometer (excitation: 594 nm, emission: 620 nm). The fluorescence values were normalized by the forward scatter of each event to account for different cell size using FlowJo Software.

Peptide-dependence growth-assay. Strains ySB270, ySB265 and ySB188 were maintained on SD agar plates supplemented with 1 μM of Ca, Bc or Vp1 peptide. For assaying their peptide-dependent growth response, strains were cultured overnight in the presence of 100 nM peptide in SC-His. Cells were washed five times with one volume of water. Cells were than seeded in 200 μl SC (no selection) at an A₆₀₀ of 0.06 and cultured at 30° C. and 800 RPM shaking. Cells were exposed to different concentrations of peptide (seven 10-fold dilutions starting from 1 μM, water was used for the “no-peptide” control). A₆₀₀ was determined at various time points over the course of 24 h. The 24 h-data points were plotted against the log₁₀ of the peptide concentrations. Data were fit to a four-parameter non-linear regression model using Prism (GraphPad) to extract values for peptide/growth EC₅₀. For dot assays, serial 10-fold dilutions of overnight cultures of ySB270 and ySB265 were spotted on SD agar plates supplemented with or without 1 μM peptide and incubated at 30° C. for 48 hours.

2-Yeast and 3-Yeast interdependent co-culturing. Strains ySB270, ySB265 and ySB188 were transformed with the appropriate peptide secretion vectors (Bc, Ca or Vp1) featuring peptide expression under the constitutive ADH1 promoter. For assaying 2-Yeast interdependence, the resulting peptide-secreting strains (treated with peptide and washed as described above) were seeded in the appropriate combination in a 1:1 ratio in 200 μl SC-His at an A₆₀₀ of 0.06 (0.03 each) and cultured at 30° C. and 800 RPM shaking. The same cell number of single strains was seeded alone and cultured in parallel as control. A₆₀₀ measurements were taken at the indicated time points and cultures were diluted into fresh media when the culture reached an A₆₀₀ of 0.8-1. For assaying 3-Yeast interdependence, the appropriate peptide secreting strains (c1, c2 and c3) were inoculated in a ratio of 1:1:1 in 200 μl SC-His media at an A₆₀₀ of 0.06 (0.02 each) in a 96-well plate cultured at 30° C. and 800 RPM shaking. Experiments were run in triplicate. All three combinations of controls lacking one essential member (c1 omitted, c2 omitted, c3 omitted) were run in parallel. A₆₀₀ measurements were taken at the indicated time points and cultures were diluted 1:20 into fresh media approximately every 12 hours. After 115 h the dilution rate was reduced to 1:20 every 24 hours. The total run time was 183 h (˜7.5 d). Samples were taken before every dilution. Samples were used to determine the co-culture composition and the peptide concentration as follows: De-convolution of strain identity: aliquots of the culture were plated on three different plate types, YPD containing either 1 μM Bc, Ca or Vp1 synthetic peptide. Each strain can only grow on plates containing its cognate peptide ligand. The co-culture composition was than determined by colony counting. Peptide concentration: JTy014 transformed with the appropriate GPCR was used as peptide sensor. The linear range of the GPCRs dose response was used for peptide quantification.

### Example 2. Language Component Acquisition Pipeline—Genome Mining Yields a Scalable Pool of Peptide/GPCR Interfaces for Synthetic Communication

Engineering multicellularity is one of the aims of Synthetic Biology¹⁻³. A bottleneck to effectively building multicellular systems can be the need for a scalable signaling language with a large number of interfaces that can be used simultaneously.

The transition from unicellular to multicellular organisms is considered one of the major transitions in evolution⁴. Phylogenetic inference suggests that cell-cell communication, cell-cell adhesion and differentiation constitute the key genetic traits driving this transition⁵. Accordingly, cell-cell communication plays an important role in many complex natural systems, including microbial biofilms^{6,7}, multi-kingdom biomes^{8,9}, stem cell differentiation¹⁰, and neuronal networks¹¹. In nature, communication between species or cell types relies on a large pool of promiscuous and orthogonal communication interfaces, acting at both short and long ranges. Signals range from simple ions and small organic molecules up to highly information-dense macromolecules including RNA, peptides and proteins. This diverse pool of signals allows cells to process information precisely and robustly, enabling the emergence of properties, fate decisions, memory and the development of form and function.

In contrast, certain previous approaches to engineering synthetic biological communication mostly rely on a single signaling modality—quorum sensing (QS), a cell density-based communication system used by many bacteria¹². The discovery of bacterial QS almost 50 years ago¹³ led to a paradigm shift in synthetic microbial ecology, enabling the engineering of systems with synthetic pattern formation¹⁴, cellular computing^{15,16,} controlled population dynamics^{17,18} and emergent properties¹⁹. QS has been exported from bacteria into plants²⁰ and mammalian cells²¹.

The major class of QS is based on diffusible acyl-homoserine lactone (AHL) signaling molecules generated by AHL synthases and AHL receptors that function as transcription factors, regulating gene expression in response to AHL signals.

While QS has been demonstrated to coordinate interactions both within a bacterial species and between species, a need exists for a method for conveying discrete and isolatable information using QS²² and it thus can be difficult to use this language for engineering scalable communities. A synthetic language should have a scalable set of independent interaction channels that do not have crosstalk.

However, the scalability of QS into many independent channels can be limited by the low information content that can be encoded in AHL signaling molecules, since these molecules are structurally and chemically simple and the receptors are known to be promiscuous.^{23,24} While crosstalk can be eliminated by receptor evolution²⁵, the AHL ligand/receptor pairs are not well suited for rapid diversification into orthogonal channels by directed evolution because the AHL biosynthesis and receptor specificity would have to be engineered in concert. As a consequence, only four AHL synthase/receptor pairs are available for synthetic communication and only three have been successfully used together²⁶; this shortage of QS interfaces limits the number of possible unique nodes in a synthetic cell community²⁴.

In addition to AHL-based QS, communication has been engineered using autoinducer peptides (AIP)²⁷ and autoinducer molecules (AI-2)²⁸ from Gram-positive bacteria. Autoinducer peptides are a class of post-translationally modified peptides sensed by a membrane-bound two-component system²⁹. AI-2 is a family of 2-methyl-2,3,3,4-tetrahydroxytetrahydrofuran or furanosyl borate diester isomers—synthesized by LuxS from S-ribosylhomocysteine followed by cyclization to the various AI-2 isoforms^{30,31}—and recognized by the transcriptional regulator LsrR³². It was shown that the response characteristics and the promoter specificity of LsrR can be engineered^{33,34} and that cell-cell communication can be tuned by using various AI-2 analogues²⁸.

However, the complexity of signal biosynthesis and reliance on specific transporters for signal import- and export³² can limit the scalability of these systems in terms of available unique communication interfaces.

Mammalian Notch receptors have been repurposed to engineer modular communication components for mammalian cells. Sixteen distinct SynNotch receptors were engineered and pairs of two where employed together³⁵; however, SynNotch receptors are contact-dependent and therefore are only suitable for short-range communication, which is conceptually different from long-range communication through diffusible signals.

Because GPCRs couple well to the conserved yeast MAP-kinase signaling cascade³⁶, it was hypothesized that the peptide/GPCR-based mating language of fungi could overcome certain limitations and be harnessed as a source of modular parts for a scalable intercellular signaling system. Fungi use peptide pheromones as signals to mediate species-specific mating reactions³⁷. These peptides are genetically encoded, translated by the ribosome, and the alpha-factor-like peptides, which are typified by the 13-mer S. cerevisiae mating pheromone alpha-factor, and are secreted through the canonical secretion pathway without covalent modifications. Peptide pheromones are sensed by specific GPCRs (e.g., Ste2-like GPCRs) that initiate fungal sexual cycles³⁸. The peptide pheromones (e.g., 9-14 amino acids in length) are rich in molecular information and the composition of peptide pheromone precursor genes is modular, consisting of two N-terminal signaling regions—“pre” and “pro”— that mediate precursor translocation into the endoplasmic reticulum and transiting to the Golgi, followed by repeats of the actual peptide sequence separated by protease processing sites. This modular precursor composition allows bioinformatic inference of mature peptide ligand sequences from available genomic databases. GPCRs from mammalian and fungal origin have been used on a small scale (two to three GPCRs) to engineer programmed behavior and communication^{39,40} and cellular computing⁴¹. However, leveraging the vast number of naturally-evolved mating peptide/GPCR pairs as a scalable signaling “language” remains an unmet need.

In order to challenge the inherent scalability of the fungal mating components as a synthetic signaling language, a pipeline for language component acquisition and communication assembly was established (FIG. 1A): An array of peptide/GPCR pairs was first genome-mined and GPCR functionality and peptide secretion was verified. Next, GPCR activation was coupled to peptide secretion to validate their functionality as orthogonal communication interfaces. Those interfaces were then used to assemble scalable communication topologies and eventually to establish peptide signal-based interdependence as a strategy to assemble stable multi-member microbial communities. As shown in FIG. 1A, the upper panel displays the mining of ascomycete genomes yields a scalable pool of peptide/GPCR pairs, the middle panel shows that GPCR activation can be coupled to peptide secretion to establish two-cell communication links. Each cell senses an incoming peptide signal via a specific GPCR, with GPCR activation leading to secretion of an orthogonal user-chosen peptide. The secreted peptide serves as the outgoing signal sensed by the second cell. The lower panel of FIG. 1A shows that scalable communication networks can be assembled in a plug- and play manner using the two-cell communication links.

First, a total of 45 peptide/GPCR pairs from available Ascomycete genomes (Table 3) was mined; sequences of mature peptide ligands were taken from literature (Table 3) or inferred from peptide precursor sequences (Table 4). In some cases, inference of mature peptide sequences was hampered by ambiguous protease processing sites or sequence-variable peptide repeats. The GPCR's tolerance to sequence variation in its peptide ligands was evaluated by incorporating alternate peptide sequence candidates into the analysis (Table 3 and 4). Functionality of heterologous mating GPCRs in S. cerevisiae requires proper insertion into the membrane and coupling to the S. cerevisiae Gα subunit (FIG. 1B). As shown in FIG. 1B, mating GPCRs couple to the S. cerevisiae Gₐₗₚₕₐ protein (Gpa1) and signals are transduced through a MAP-kinase-mediated phosphorylation cascade. Gene activation can then be mediated by the transcription factor Ste12 through binding of a pheromone response element (PRE, grey) in the promoters of mating-associated genes (e.g., FUS1 and FIG1, used herein to control synthetic constructs of choice). Peptides are translated by the ribosome as pre-pro peptides. Pre-pro peptide architecture is conserved and starts with an N-terminal secretion signal (light blue), followed by Kex2 and Ste13 recognition sites (grey and yellow, respectively). Mature secreted peptides (red) are processed while trafficking through the ER and Golgi. The conserved pre-pro peptide architecture enables the bioinformatic de-orphanization of fungal GPCRs by inference of mature peptide sequences from precursor genes.

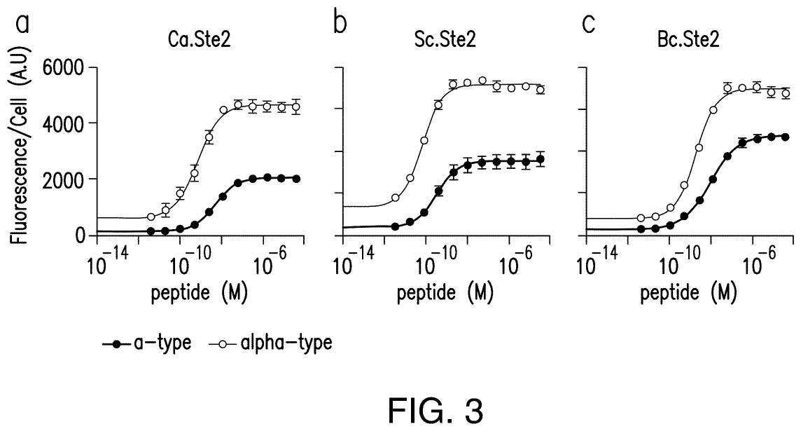

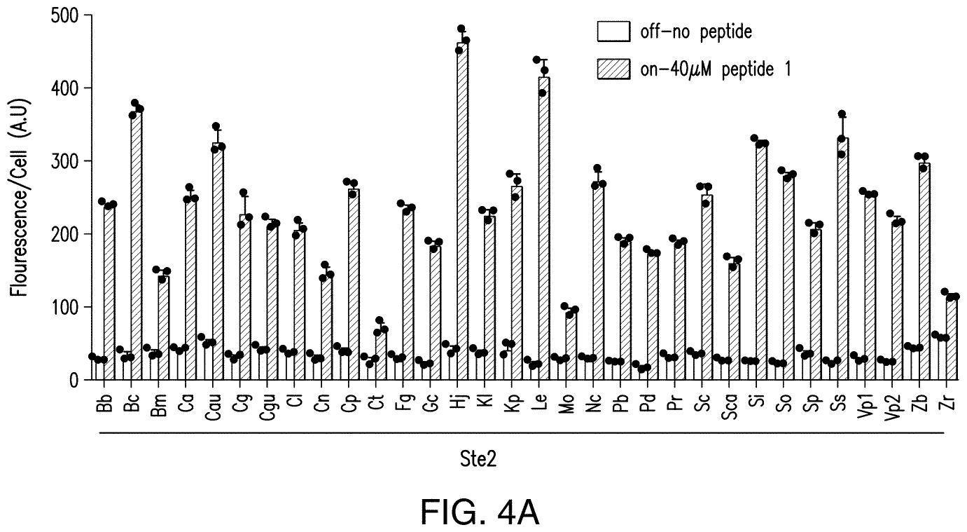

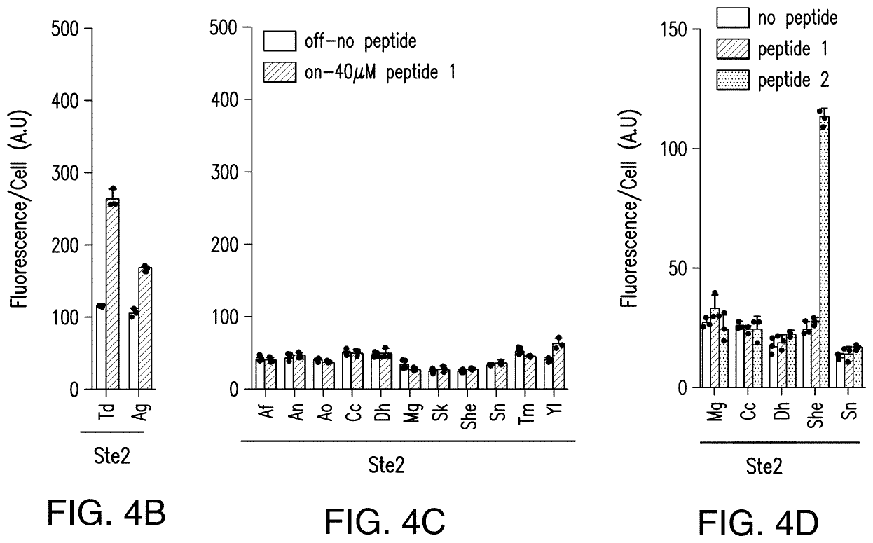

Genome-mined GPCRs showed amino acid sequence identities between 17-68% to the S. cerevisiae mating GPCR Ste2 (Table 3), but most of them showed higher conservation at specific intracellular loop motifs known to be important for Gα coupling^{42,43} (FIG. 2, Table 3). A detailed view of the receptor topology with seven transmembrane helixes is provided in panel a of FIG. 2 with key regions involved in signaling highlighted in green and blue. Panels b and c of FIG. 2 show residue conservation among the herein reported fungal GPCRs for the regions highlighted in green and blue in panel a. Functionality of peptide/GPCR pairs was assessed in a standardized workflow, in which codon-optimized GPCR genes were expressed in S. cerevisiae and tested for a positive response to synthetic peptide ligands using a FUS1 promoter inducible red fluorescent protein (yEmRFP⁴⁴) signal as a read-out. The simple chemistry of the peptide ligand synthesis facilitated GPCR characterization, as any short peptide sequence is readily commercially available. GPCRs were expressed from the TDH3 promoter using a low-copy plasmid. A read-out strain was engineered for a fluorescence assay by deleting both endogenous mating GPCR genes (STE2 and STE3), all pheromone genes (MFA1/2 and MFALPHA1/MFALPHA2), BAR1 and SST2 to improve pheromone sensitivity, and FAR1 to avoid growth arrest (Table 2). The read-out strain was constructed in both mating type genetic backgrounds. Although the MATa-type was used for language characterization herein, language functionality in the MATα-type was confirmed using a subset of GPCRs (FIG. 3). As shown in FIG. 3, the functionality of three peptide/GPCR pairs was verified in both mating-types (Panel a: Ca. Ste2; Panel b: Sc.Ste2; Panel c: Bc.Ste2). Strain yNA899 (a-type) and yNA903 (alpha-type) were transformed with the appropriate GPCR expression constructs as well as with a plasmid encoding for a FUS1p-controlled red fluorescent read-out.

Remarkably, 32 out of 45 tested GPCRs (73%) gave a strong fluorescence signal in response to their inferred synthetic peptide ligand (ligand candidate #1, Table 3 and 4) (FIG. 1C, FIG. 18A). The functionality of 45 peptide/GPCR pairs was evaluated by on/off testing using 40 μM cognate peptide and fluorescence as read-out. GPCRs are organized by percent amino acid identity to the Sc. Ste2., and non-functional GPCRs (those that give a signal difference <3 standard deviations) are highlighted in red; constitutive GPCRs are highlighted in green (FIG. 1C). Two GPCRs were constitutively active and showed fluorescence levels >three-fold above the basal levels of the other GPCRs in the absence of peptide, but showed an increase in activation in the presence of peptide (FIG. 1C, FIG. 18B). 11 GPCRs did not respond to the initially inferred peptide ligand candidates (FIG. 1C, FIG. 18C). One of these 11 GPCRs (She. Ste2) can be activated when using an alternate near-cognate peptide ligand candidate (in this specific case the near-cognate candidate has two additional N-terminal residues), indicating that the wrong peptide sequence was initially inferred (FIG. 18D).

### Example 3. Synthetic Language Characterization—Peptide/GPCR Pairs Cover a Wide Range of Tunable Response Characteristics, they are Naturally Orthogonal and Peptides are Functionally Secreted

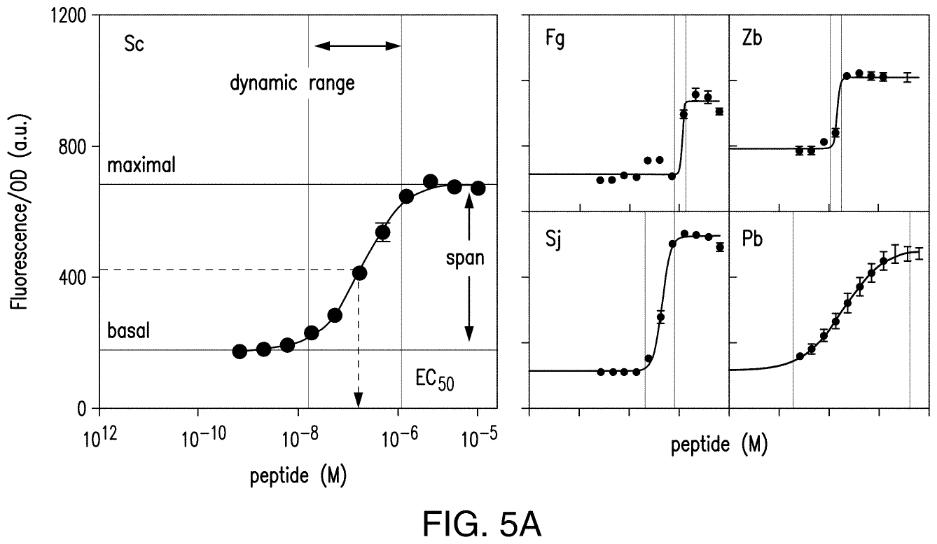

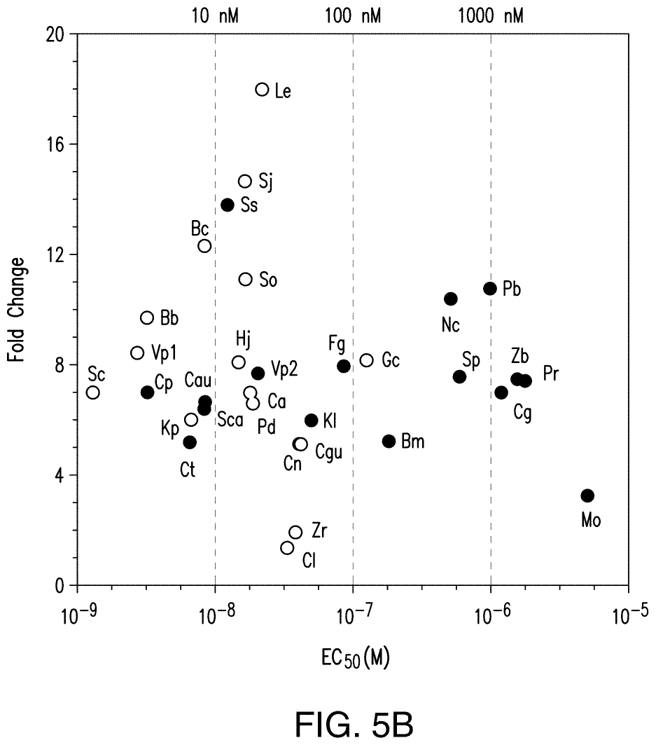

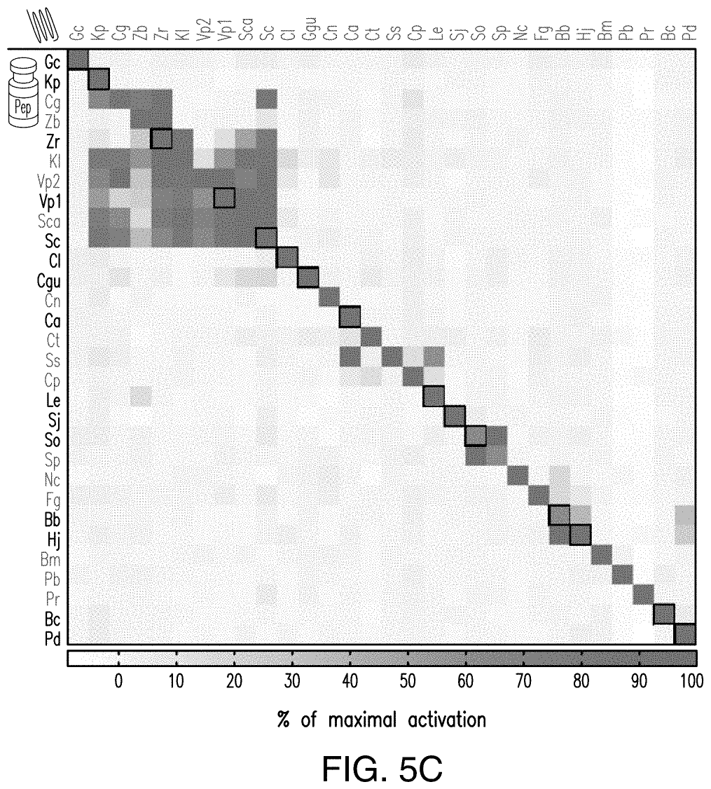

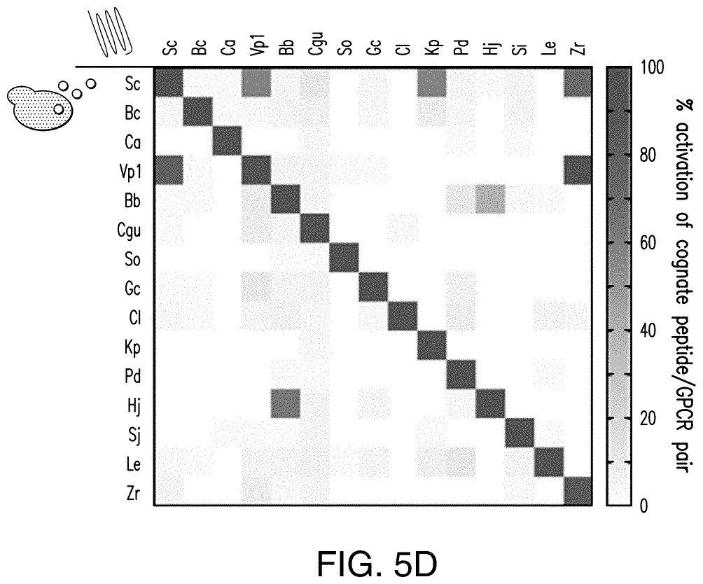

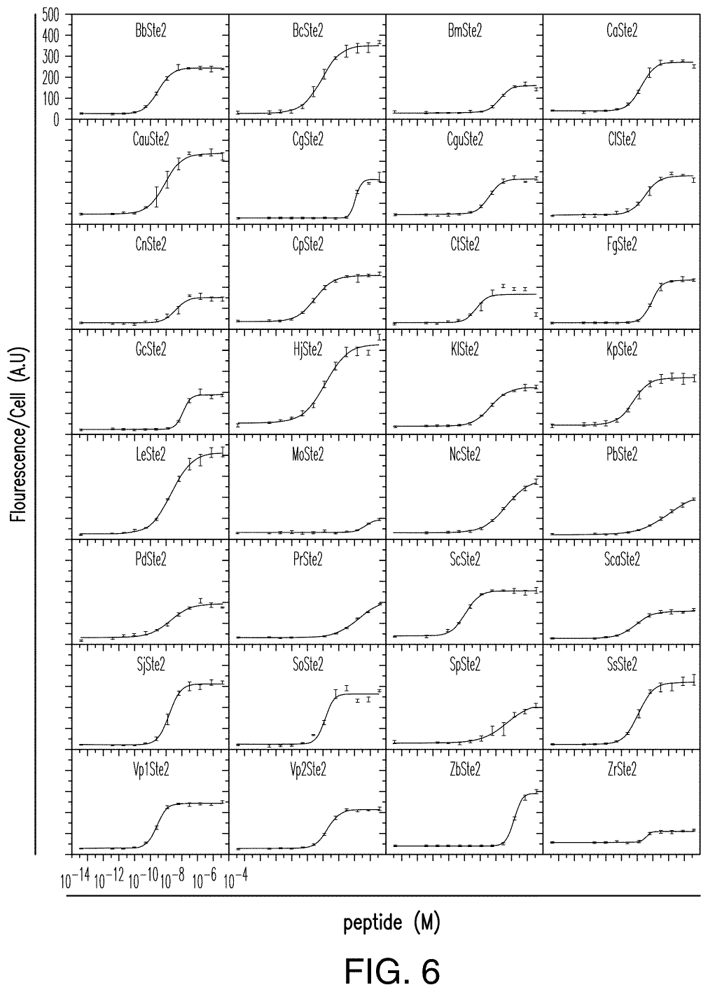

After initial on/off screening, dose-response curves were measured for all 32 functional GPCRs and extracted parameters crucial for establishing communication: Sensitivity of GPCRs (EC₅₀), basal and maximal activation (fold-change activation), dynamic range (Hill coefficient), orthogonality, reversibility of signaling, and population response behavior (FIG. 5A, FIG. 5B, FIG. 5C, FIG. 6, Table 6). FIG. 5A shows the performance of each peptide/GPCR pair by recording its dose-response to synthetic cognate peptides, using fluorescence as a read-out. The dose-response curves of exemplary GPCRs (Sc.Ste2, Fg.Ste2, Zb.Ste2, Sj.Ste2, Pb.Ste2) with different response behaviors are featured in FIG. 5A. FIG. 5B shows the EC₅₀ values of peptide/GPCR pairs, which are summarized in Table 6. FIG. 5C provides a 30×30 orthogonality matrix that was generated by testing the response of 30 GPCRs across all 30 peptide ligands and shows that GPCRs are naturally orthogonal across non-cognate synthetic peptide ligands. The test concentration used in the experiments of FIG. 5C, which were performed in triplicate, was set at 10 μM of a given peptide ligand. The fluorescence signal for maximum activation of each GPCR (not necessarily its cognate ligand) was set to 100% activation and the threshold for categorizing cross-activation was set to be ≥15% activation of a given GPCR by a non-cognate ligand.

TABLE 6
peptide/GPCR pair characteristics: Parameters were extracted from the dose response
curves given in FIG. 6 by fitting them to a 4-parameter model using Prism GraphPad.
Errors represent the standard error of the curve generated from triplicate values,
except for fold change error, which was propagated from the Top and Bttm errors. Peptide/GPCR
pairs are ordered alphabetically according to the 2-letter species code.

| | | | | | | | | | | Fold | | Hill |
|---|---|---|---|---|---|---|---|---|---|---|---|---|
| | | EC50 | | Top | | Bttm | | Span | Fold | Change | Hill | Slope |
| Code | EC50 | error | Top | error | Bttm | error | Span | error | Change | error | Slope | error |
| Bb | −8.5 | 0.0 | 244.1 | 2.5 | 25.2 | 2.8 | 218.9 | 3.9 | 9.7 | 1.1 | 1.0 | 0.1 |
| Bc | −8.1 | 0.1 | 351.9 | 5.8 | 28.6 | 5.3 | 323.3 | 8.6 | 12.3 | 2.3 | 0.7 | 0.1 |
| Bm | −6.7 | 0.1 | 158.8 | 3.3 | 30.3 | 1.9 | 128.4 | 3.9 | 5.2 | 0.3 | 1.2 | 0.2 |
| Ca | −7.7 | 0.0 | 271.6 | 3.8 | 38.9 | 3.1 | 232.8 | 5.1 | 7.0 | 0.6 | 1.0 | 0.1 |
| Cau | −8.1 | 0.1 | 336.9 | 6.7 | 50.6 | 6.2 | 286.3 | 9.8 | 6.7 | 0.8 | 0.8 | 0.1 |
| Cg | −5.9 | 0.0 | 213.6 | 4.0 | 30.5 | 1.9 | 183.0 | 4.5 | 7.0 | 0.5 | 2.4 | 0.5 |
| Cgu | −7.4 | 0.0 | 211.7 | 2.7 | 41.2 | 2.0 | 170.5 | 3.5 | 5.1 | 0.3 | 1.1 | 0.1 |
| Cl | −7.5 | 0.1 | 225.8 | 4.4 | 39.8 | 3.2 | 186.0 | 5.8 | 1.4 | 0.1 | 0.9 | 0.1 |
| Cn | −7.4 | 0.1 | 152.2 | 4.2 | 29.7 | 3.0 | 122.5 | 5.4 | 5.1 | 0.5 | 1.1 | 0.2 |
| Cp | −8.5 | 0.0 | 254.0 | 2.7 | 36.2 | 3.0 | 217.8 | 4.3 | 7.0 | 0.6 | 0.8 | 0.1 |
| Ct | −8.2 | 0.2 | 166.7 | 10.1 | 32.0 | 10.0 | 134.6 | 14.7 | 5.2 | 1.6 | 1.2 | 0.6 |
| Fg | −7.1 | 0.0 | 232.2 | 2.5 | 29.2 | 1.6 | 203.0 | 3.0 | 8.0 | 0.4 | 1.3 | 0.1 |
| Gc | −6.9 | 0.0 | 187.2 | 2.8 | 22.9 | 1.8 | 164.3 | 3.4 | 8.2 | 0.7 | 1.8 | 0.2 |
| Hj | −7.8 | 0.1 | 429.5 | 9.3 | 53.0 | 7.3 | 376.5 | 13.2 | 8.1 | 1.1 | 0.6 | 0.1 |
| Kl | −7.3 | 0.0 | 223.1 | 2.8 | 37.2 | 1.8 | 185.9 | 3.6 | 6.0 | 0.3 | 0.8 | 0.0 |
| Kp | −8.2 | 0.1 | 269.1 | 4.4 | 44.8 | 4.2 | 224.3 | 6.5 | 6.0 | 0.6 | 0.8 | 0.1 |
| Le | −7.7 | 0.1 | 412.5 | 6.4 | 22.9 | 4.7 | 389.6 | 8.8 | 18.0 | 3.7 | 0.7 | 0.1 |
| Mo | −5.3 | 0.1 | 97.6 | 5.5 | 29.9 | 1.0 | 67.7 | 5.7 | 3.3 | 0.2 | 1.2 | 0.2 |
| Nc | −6.3 | 0.1 | 286.7 | 6.4 | 27.6 | 1.7 | 259.2 | 7.2 | 10.4 | 0.7 | 0.6 | 0.0 |
| Pb | −6.0 | 0.1 | 217.1 | 9.3 | 20.2 | 1.6 | 196.9 | 10.1 | 10.8 | 1.0 | 0.5 | 0.0 |
| Pd | −7.7 | 0.1 | 190.0 | 5.2 | 28.8 | 4.0 | 161.2 | 7.2 | 6.6 | 0.9 | 0.7 | 0.1 |
| Pr | −5.8 | 0.1 | 207.3 | 7.3 | 27.9 | 1.1 | 179.4 | 7.7 | 7.4 | 0.4 | 0.6 | 0.0 |
| Sc | −8.9 | 0.0 | 253.1 | 2.2 | 36.2 | 2.8 | 217.0 | 3.8 | 7.0 | 0.5 | 1.0 | 0.1 |
| Sca | −8.1 | 0.0 | 155.4 | 1.9 | 24.3 | 1.7 | 131.1 | 2.8 | 6.4 | 0.5 | 0.7 | 0.1 |
| Sj | −7.8 | 0.0 | 311.3 | 3.7 | 21.2 | 3.1 | 290.0 | 5.1 | 14.7 | 2.2 | 1.2 | 0.1 |
| So | −7.8 | 0.1 | 263.4 | 6.2 | 23.7 | 5.5 | 239.7 | 5.5 | 11.1 | 2.6 | 1.5 | 0.4 |
| Sp | −6.2 | 0.2 | 224.3 | 16.7 | 29.6 | 3.9 | 194.7 | 3.9 | 7.6 | 1.1 | 0.5 | 0.1 |
| Ss | −7.9 | 0.1 | 318.0 | 5.0 | 23.0 | 4.4 | 295.0 | 7.0 | 13.8 | 2.6 | 0.9 | 0.1 |
| Vp1 | −8.6 | 0.0 | 243.1 | 1.7 | 28.8 | 1.9 | 214.2 | 2.6 | 8.4 | 0.5 | 1.4 | 0.1 |
| Vp2 | −7.7 | 0.0 | 215.2 | 1.8 | 28.0 | 1.5 | 187.2 | 2.4 | 7.7 | 0.4 | 1.1 | 0.1 |
| Zb | −5.8 | 0.0 | 292.5 | 3.5 | 39.1 | 1.3 | 253.4 | 3.9 | 7.5 | 0.3 | 1.7 | 0.1 |
| Zr | −7.4 | 0.1 | 109.9 | 1.4 | 57.2 | 1.2 | 52.7 | 1.9 | 1.9 | 0.0 | 2.4 | 0.6 |

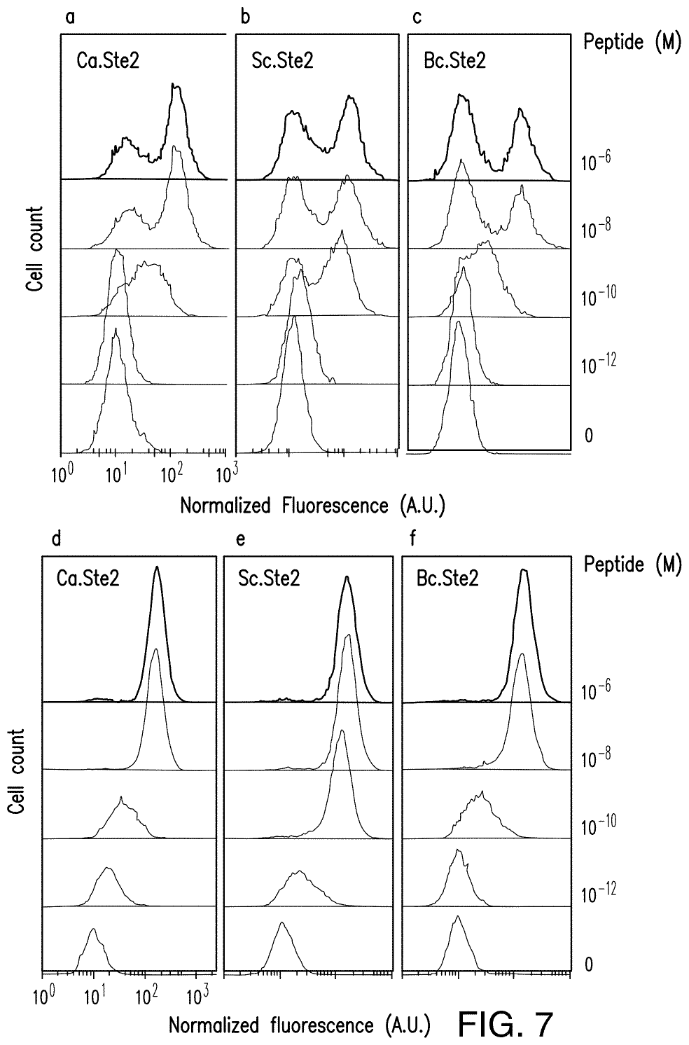

Sensitivity of the GPCRs for their cognate ligand gave an EC₅₀ range of ˜1 to 10⁴ nM, with the natural S. cerevisiae Ste2 exhibiting the highest sensitivity of 1.25 nM. This is comparable to the sensitivity of available QS systems²⁶. Functional GPCRs displayed between 1.3 and 17-fold activation. This range overlaps that of QS systems but is on average slightly lower than available QS systems²⁶ but comparable to other engineered GPCR-based signaling systems in yeast and mammalian cells^{45,46} Response behaviors ranged from a graded response (analog) with a wide dynamic range to “switch-like” (digital) behavior with a very narrow dynamic range. When dose responses were characterized at the single-cell level, a subset of non-responding cells were observed, likely due to plasmid copy number noise (FIG. 7: panels a-c). As represented in panels a-c of FIG. 7, GPCRs are encoded on low copy plasmids and the fluorescent read-out is integrated on the chromosome (HO locus) (panel a shows JTy014 with pMJ90 (Ca. Ste2), panel b shows JTy014 with pMJ93 (Sc.Ste2) and panel c shows JTy014 with pMJ95 (Bc.Ste2)). Genomic integration of the GPCRs abolished this non-responding sub-population (FIG. 7: panels d-f). As represented in panels d-f of FIG. 7, both, GPCRs and the red fluorescent readout are integrated on the chromosome (panel d shows ySB98 with chromosomally integrated Ca.Ste2, panel e shows ySB99 with chromosomally integrated Sc.Ste2 and Panel f shows ySB100 with chromosomally integrated Bc.Ste2).

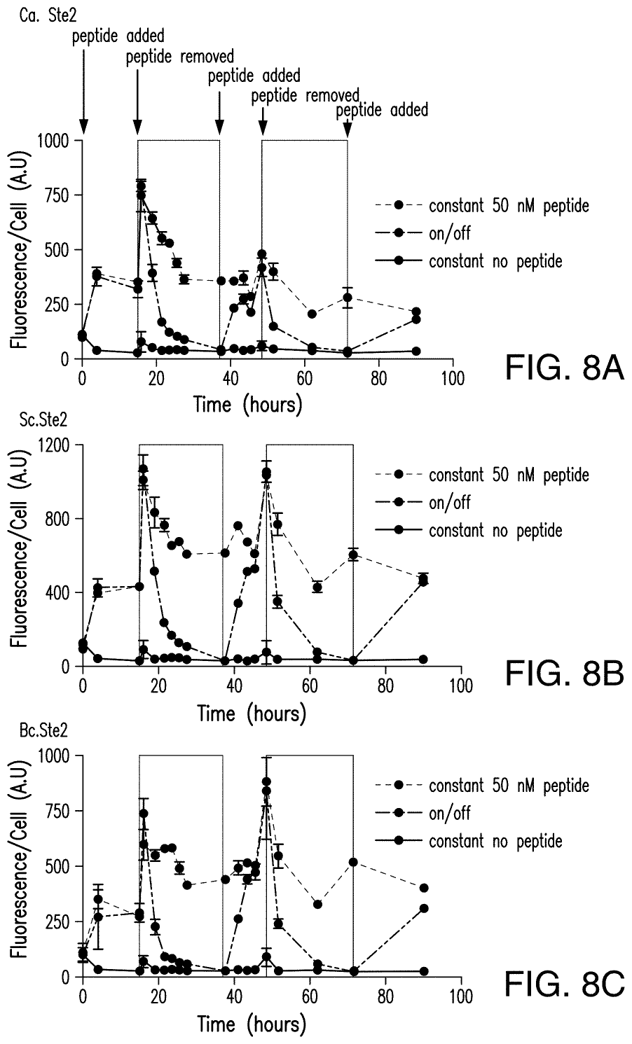

Importantly, GPCR signaling can be de-activated and re-activated several times with either no or minimal lengthening of response time (FIG. 8). As shown in FIG. 8, all strains carry the indicated GPCR and a FUS1p-controlled red fluorescent read-out on the chromosome. Panel a of FIG. 8 shows ySB98 with chromosomally integrated Ca.Ste2. Panel b of FIG. 8 shows ySB99 with chromosomally integrated Sc.Ste2. Panel c of FIG. 8 shows ySB100 with chromosomally integrated Bc.Ste2. At time point zero, GPCRs were activated with 50 nM peptide. After reaching sufficient induction, cells were washed with water to remove the peptide. Cells were re-seeded and grown until the fluorescence level went back to baseline. After reaching baseline, cells were re-induced with 50 nM peptide. Positive and negative controls using cells constantly exposed to 50 nM peptide and cells not exposed to peptide were run simultaneously. Experiments were performed in 96-well plates (200 μl total culturing volume) and run in triplicates.

The GPCRs can also be co-expressed in a single cell in order to allow for processing of two separate signals by a single cell (FIG. 9). Strain ySB315 (C1.Ste2 and Sj.Ste2) (Panel a of FIG. 9) and ySB316 (Bc.Ste2 and So.Ste2) (panel b of FIG. 9) were transformed with pSB14 (encoding for a FUS1 promoter-controlled yEmRFP read out). Each strain was tested with each individual cognate synthetic peptide as well as concurrent activation with both cognate peptides. GPCR activation was monitored by induction of a red fluorescent reporter gene under the control of the FUS1 promoter. Data were collected after 8 hours. Experiments were run in triplicates.

Next, pairwise orthogonality was assessed for a subset of 30 peptide/GPCR by exposing each GPCR to all non-cognate peptide ligands. The GPCRs showed a remarkable level of natural orthogonality (FIG. 5C). In total 14 out of 30 GPCRs were orthogonal and only activated by their cognate peptide ligand. Five GPCRs were activated by only one additional non-cognate peptide and 11 GPCRs were activated by several non-cognate ligands. The test concentration for assessing pair orthogonality was set at 10 μM of a given peptide ligand and the threshold for categorizing cross-activation was set to be ≥15% activation of a given GPCR by a non-cognate ligand (maximum activation of each GPCR at the same concentration of the cognate ligand was set to 100% activation). The selected test concentration of 10 μM is an order of magnitude higher than typically achieved by peptide secretion (1-10 nM); it would be a stringent selection criterion to yield peptide/GPCR pairs that would be fully orthogonal within the language. Typical values of cross activation were between 16 and 100%. Taken together, these data indicate a matrix of 17 fully orthogonal peptide/GPCR interfaces within the design constraints (17 receptors each orthogonal to all 16 non-cognate ligands) (FIG. 10).

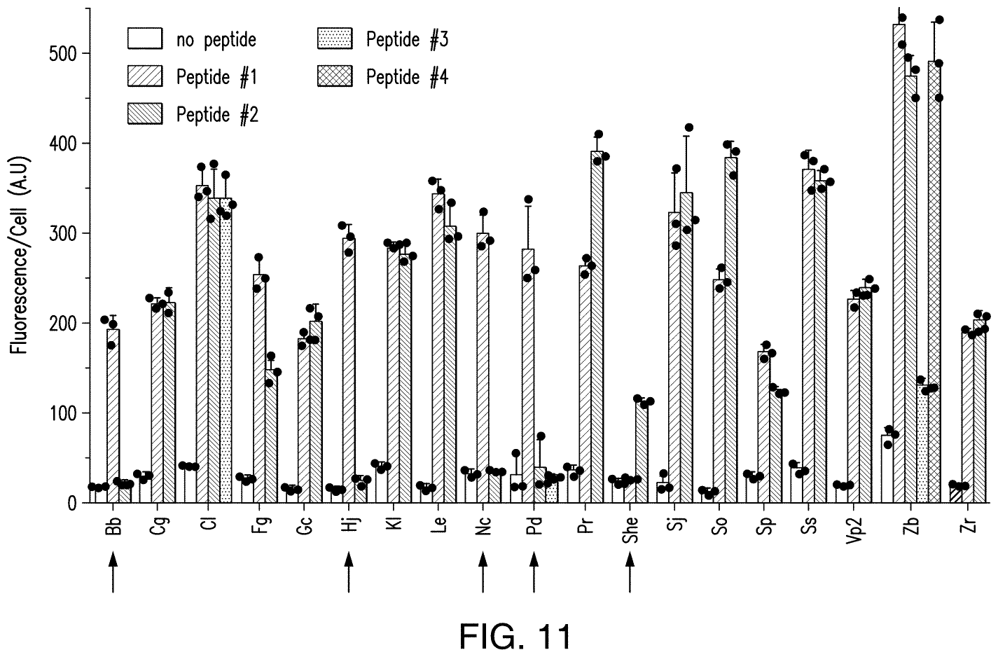

Next, the robustness of the ability to infer a GPCR's peptide ligand was validated. Thus, dose-response curves were recorded for a subset of 19 GPCRs to possible alternative near-cognate peptide ligand candidates. 14 out of the 19 GPCRs were also activated by these near-cognate peptides (FIG. 11), suggesting that the employed bioinformatic ligand inference strategy did not require precise interpretation of the exact precursor processing. As represented in FIG. 11, JTy014 was transformed with the appropriate GPCR expression construct and cells were cultured in the absence or presence of 40 μM synthetic peptide ligand. OD₆₀₀ and red fluorescence was recorded after 8 hours, experiments were performed in 96-well plates (200 μl total culture volume) and experiments were run in triplicates.

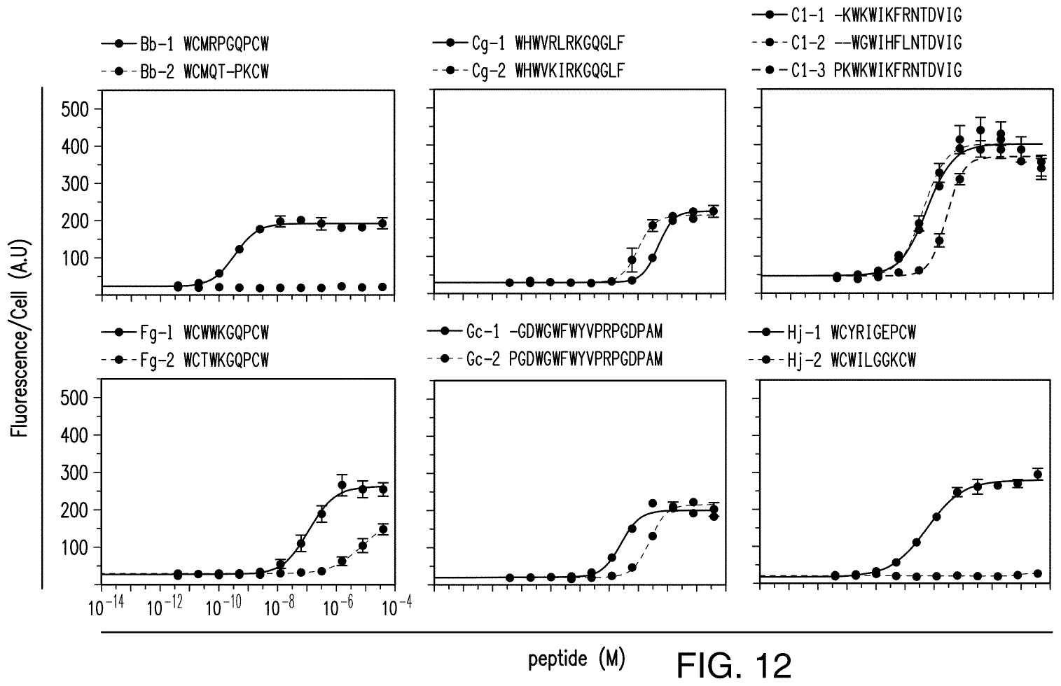

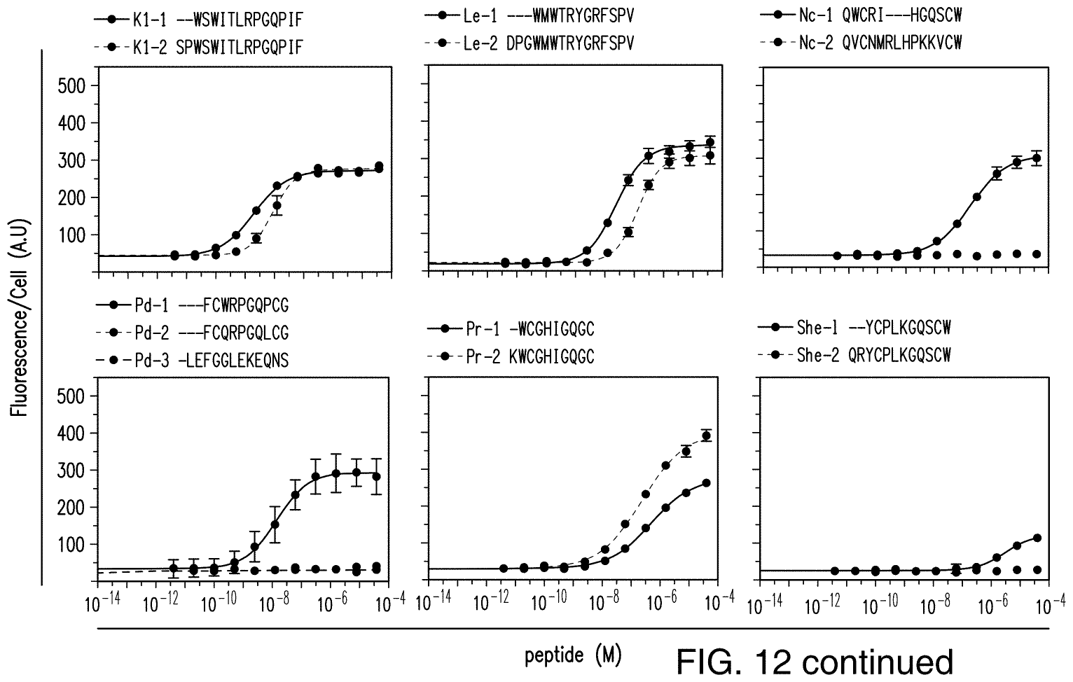

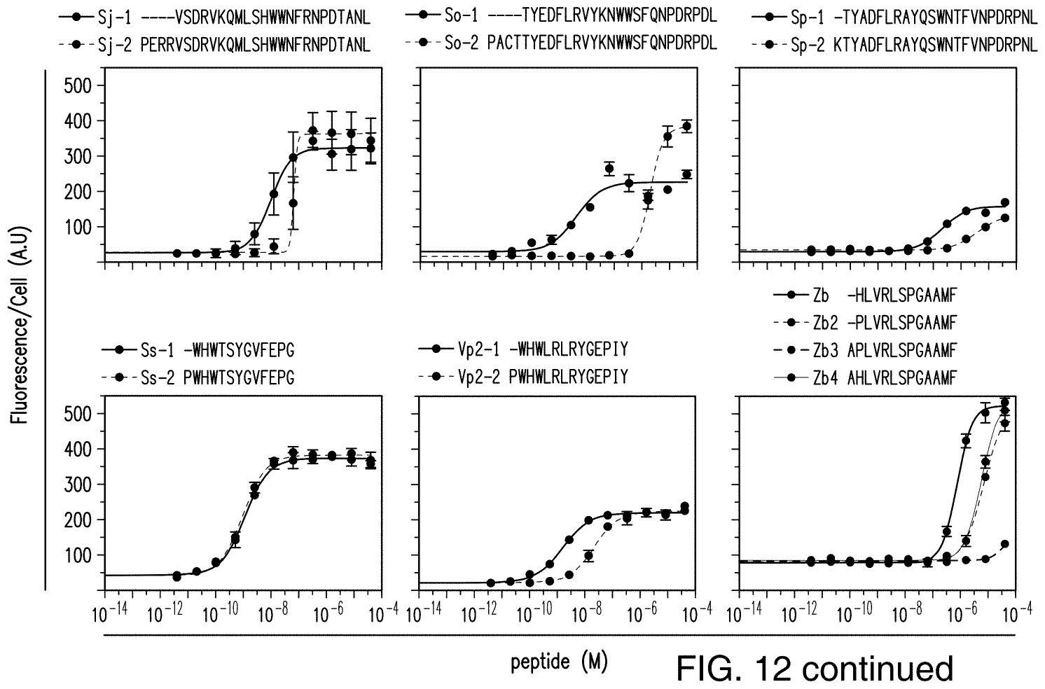

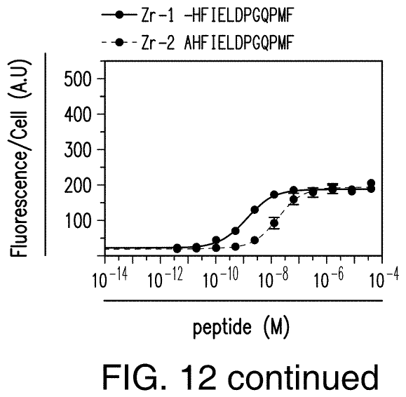

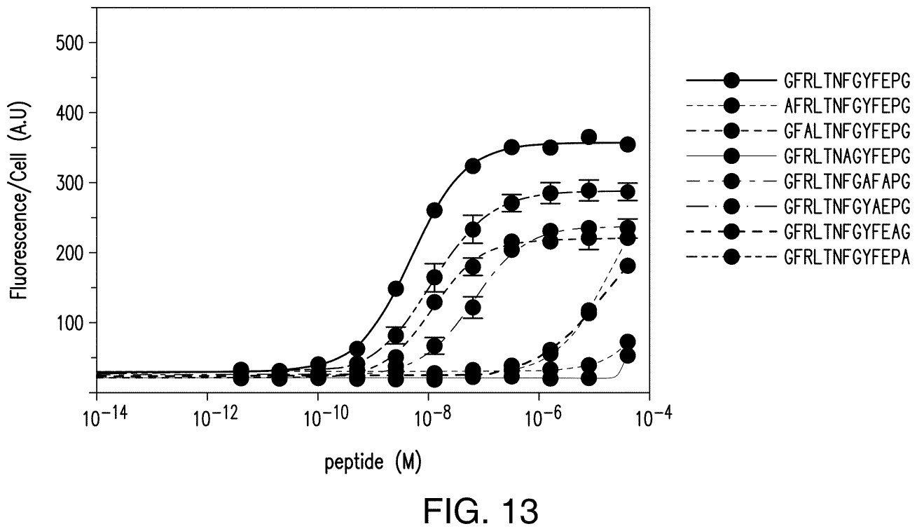

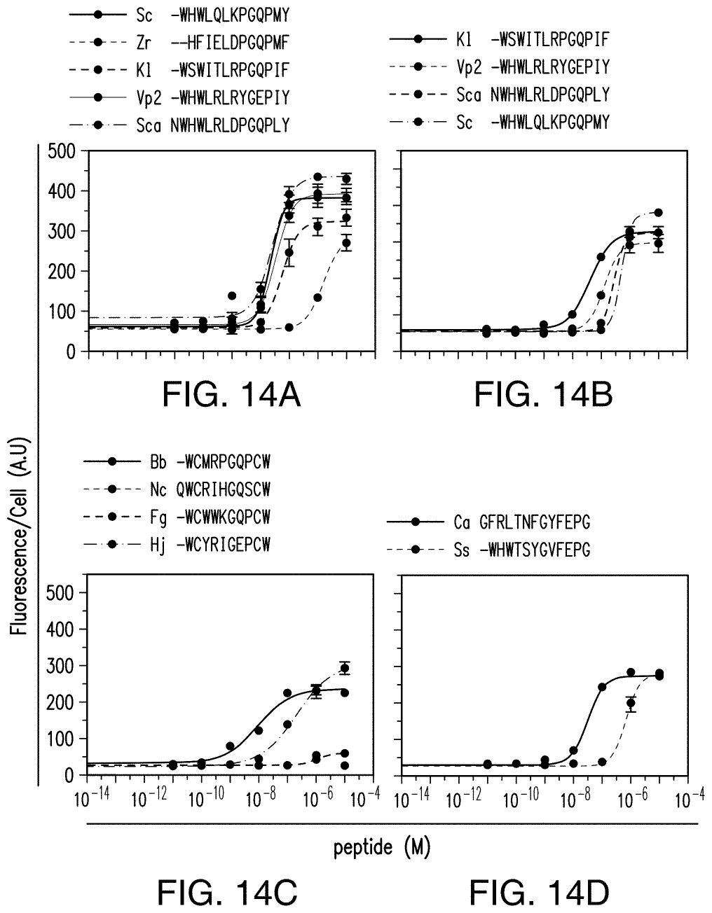

In fact, near-cognate ligands can be harnessed to induce significant changes in EC₅₀, fold activation, and dynamic range for most peptide/GPCR pairs (FIG. 12). As represented in FIG. 12, strain JTy014 was transformed with the appropriate GPCR expression constructs and each strain was tested with the indicated synthetic peptide ligands. GPCR activation was monitored by activation of a red fluorescent reporter gene under the control of the FUS1 promoter, data were collected after 12 hours and experiments were run in triplicates. For example, the So.Ste2 changed its response characteristics from gradual to switch-like when three additional residues were included at the N-terminus of its peptide. The degree and nature of changes was unique to each GPCR/peptide pair (FIG. 12). This feature was explored further by alanine scanning the peptide ligand of the Ca.Ste2. These simple one-residue exchanges elicited shifts in EC₅₀ and fold change (FIG. 13). This was further extended to several promiscuous GPCRs and their cross-activating non-cognate ligands (FIG. 14). While some GPCRs retained stable response parameters across a variety of peptide ligands, most GPCRs' response parameters can be modulated when exposed to these variant peptides. Combined, these data support contemplation of tuning the response characteristics of a given GPCR by simply recoding the peptide ligand instead of engineering the receptor itself.

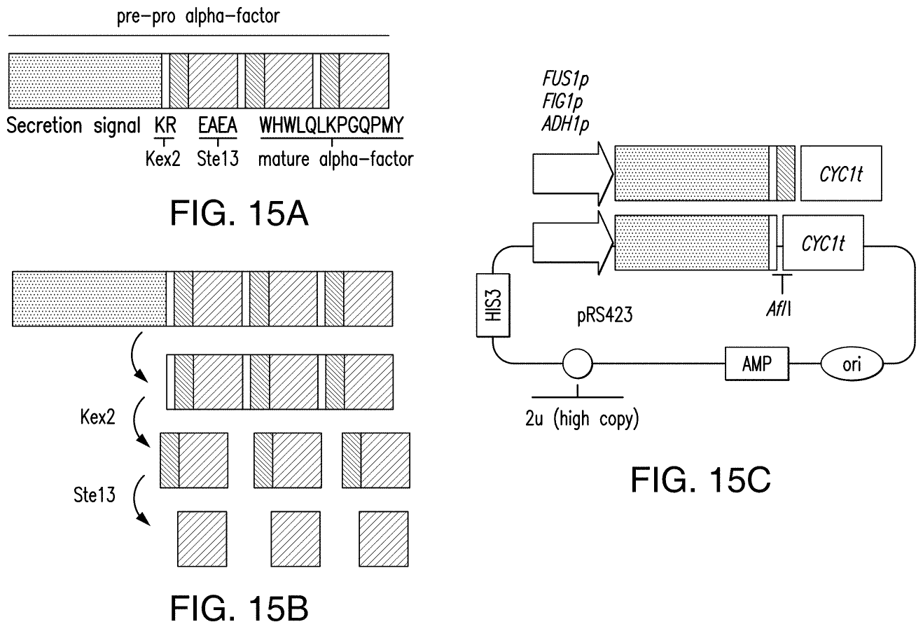

After assessing peptide/GPCR functionality with synthetic peptides, it was tested whether the peptides can be functionally secreted. The feasibility of peptide secretion from S. cerevisiae through its conserved sec pathway has been shown before,⁴⁷ but the feasibility across a wide sequence space was unclear. The amino acid sequences of 15 peptides were cloned into a peptide secretion vector, designed based on the alpha-factor pre-pro-peptide architecture (FIG. 15, Table 7). These 15 peptides were chosen based on the favorable dose-response characteristics (low EC₅₀ and high fold-change) of the corresponding peptide/GPCR pairs. A schematic representation of the S. cerevisiae alpha-factor precursor architecture with the secretion signal (blue), Kex2 (grey) and Ste13 (orange) processing sites and three copies of the peptide sequence (red) is provided in panel a of FIG. 15. Panel b of FIG. 15 provides an overview on pre-pro-peptide processing, resulting in mature alpha-factor and panel c of FIG. 15 provides a schematic representation of the peptide acceptor vector. The peptide expression cassette includes either a constitutive promoter (ADH1p) or a peptide-dependent promoter (FUS1p or FIG1p), the alpha-factor pro sequence with or without the Ste13 processing site, a unique (AflII) restriction site for peptide swapping and a CYC1 terminator (FIG. 15).

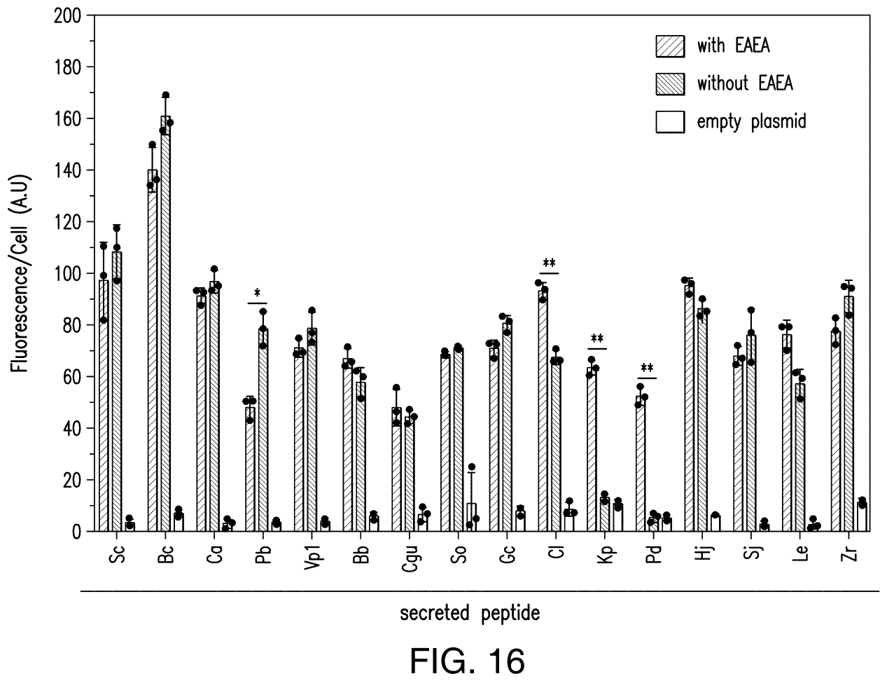

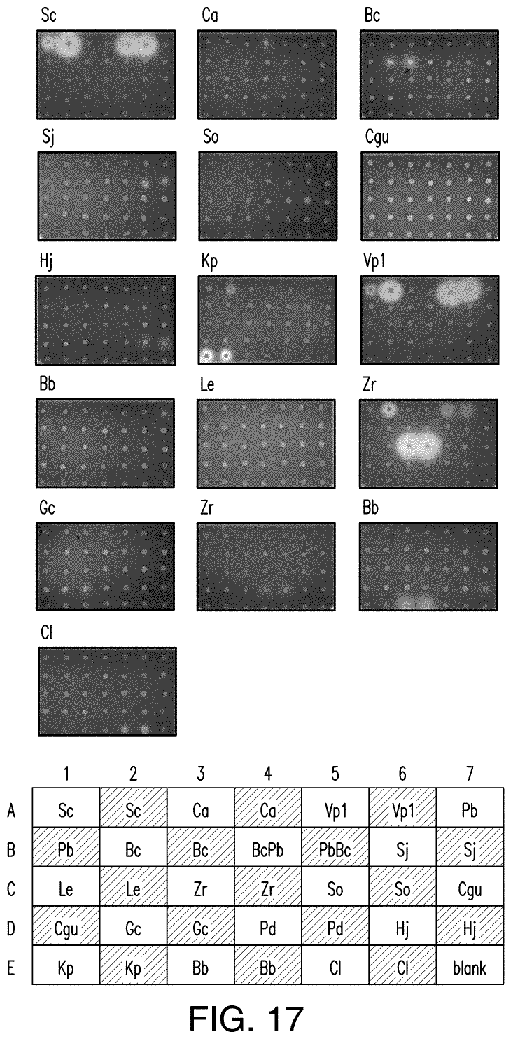

To test for peptide secretion, the appropriate GPCR/fluorescent-readout strains were employed as peptide sensors in a liquid assay as well as a fluorescent halo assay. All peptides can be secreted from S. cerevisiae (FIG. 5D, FIGS. 16 and 17) but the amount of peptide secretion was dependent on the peptide sequence (FIGS. 16 and 17). Combinatorial co-culturing of secreting and sensing strains validated that peptide/GPCR pair orthogonality was retained when peptides were secreted (FIG. 5D).

### Example 4. Synthetic Microbial Communication—Two-Cell Communication Links can be Used to Build Various Communication Topologies

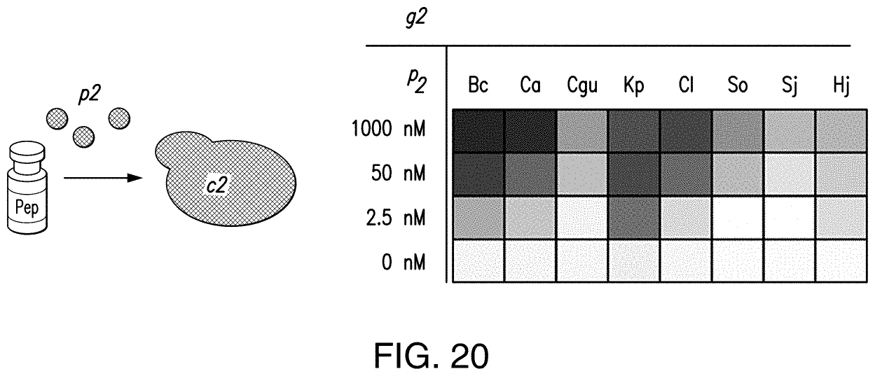

Next, functional communication was established by coupling GPCR signaling to peptide secretion. The language was conceptualized to be built from two-cell links as the minimal signaling units that can be easily characterized and assembled into higher-order communication topologies (FIG. 18A). In brief, in a c1-c2 two-cell link, Cell 1 (c1) senses synthetic peptide through GPCR 1 (g1). Activation of g1 leads to secretion of peptide 2 (p2). p2 is sensed by cell 2 (c2) through GPCR 2 (g2). g2 activation is coupled to a fluorescent read-out. Signal transmission from c1 to c2 can be assessed by recording transfer functions using co-cultures of c1 and c2. c1 is exposed to increasing concentrations of synthetic p1 and an increase in fluorescence of c2 (by virtue of GPCR g2 signaling) is recorded as a read-out. Dose-dependent transfer of information through each link can be assessed by exposing cell c1 to an increasing dose of synthetic peptide p1 and measuring an increase in fluorescence in cell c2. In this manner, each two-cell link can be characterized by a signal transfer function (p1 dose to c2 response) making it easy to identify optimal links for a given topology. In order to test the assembly of functional two-cell links, eight fully-orthogonal peptide/GPCR pairs were chosen and the complete combinatorial set of 56 possible links characterized (all possible non-cognate combinations; FIG. 18A and FIG. 18B, FIGS. 19 and 20). As shown in FIG. 18B, eight GPCRs at the g1 position were coupled to secretion of the seven non-cognate peptides at the p2 position. Data were organized by the GPCR at the g1 position. Each GPCR was coupled to secretion of all seven non-cognate p2's. Heat-maps show the fluorescence value of c2 after exposing c1 to increasing doses of p1 (FIG. 18B). In all 56 cases, activation of the g1 GPCR resulted in a graded, p1 concentration-dependent fluorescence signal in c2.

Next it was tested if the language can be used to link multiple yeast strains and build synthetic multicellular communities. The functional capabilities of single engineered organisms are limited by their capacity for genetic modification. Multi-membered microbial consortia engineered to cooperate and distribute tasks show promise to unlock this constraint in engineering complex behavior. For example, engineering sense-response consortia composed of yeast that sense a trigger, e.g., a pathogen³⁶, and yeast that respond, e.g., by killing the pathogen through secretion of an antimicrobial⁴⁸ is contemplated. Further, consortia have shown distinct advantages for metabolic engineering, such as distribution of metabolic burden, as well as parallelized, modular optimization and implementation^{49,50}. Those consortia have applications in degrading complex biopolymers like lignin, cellulose⁵¹ or plastic⁵².

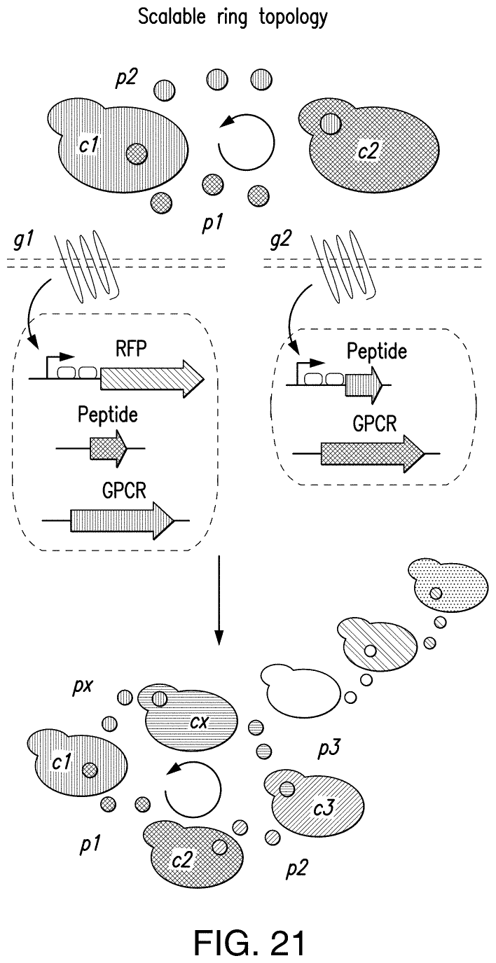

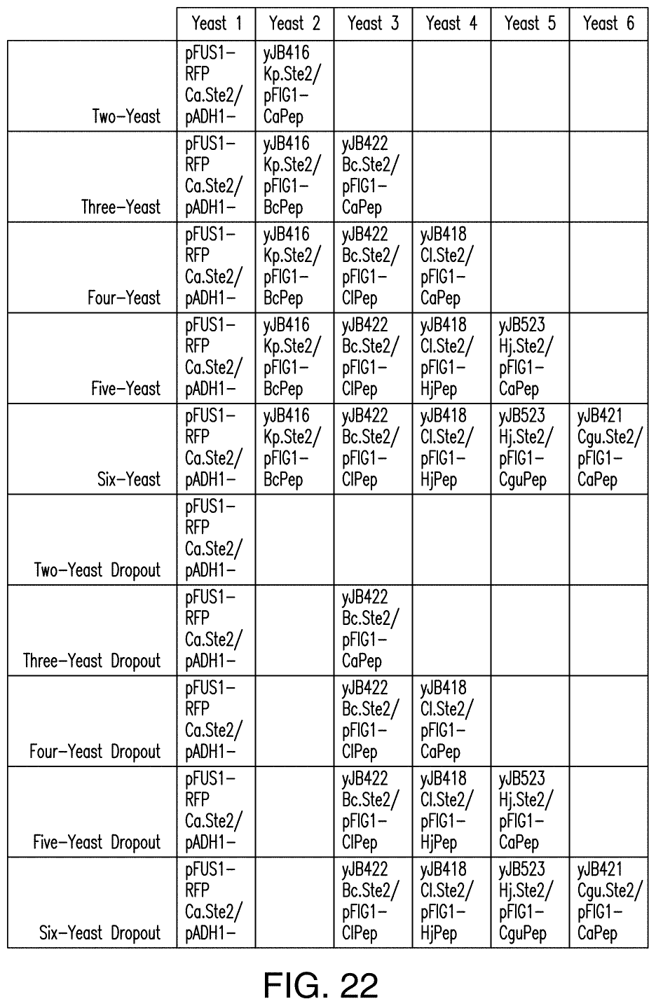

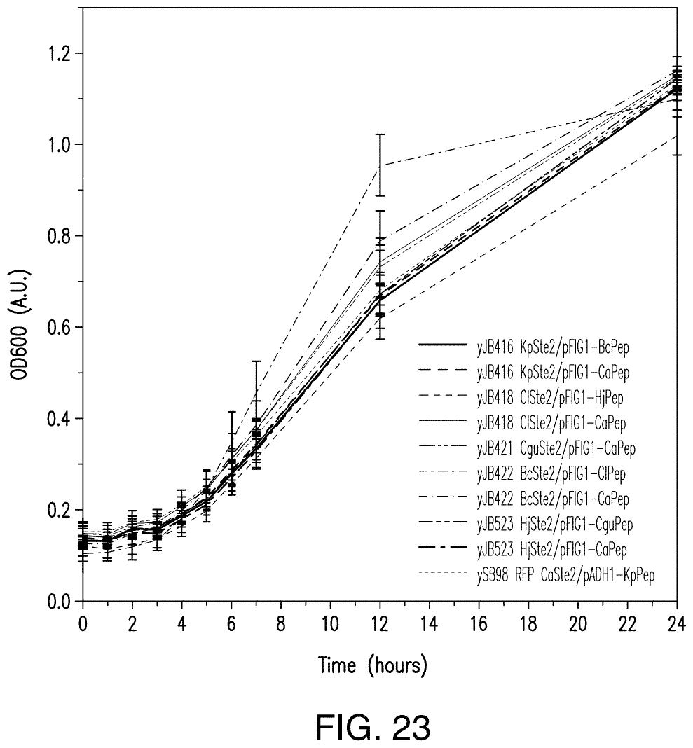

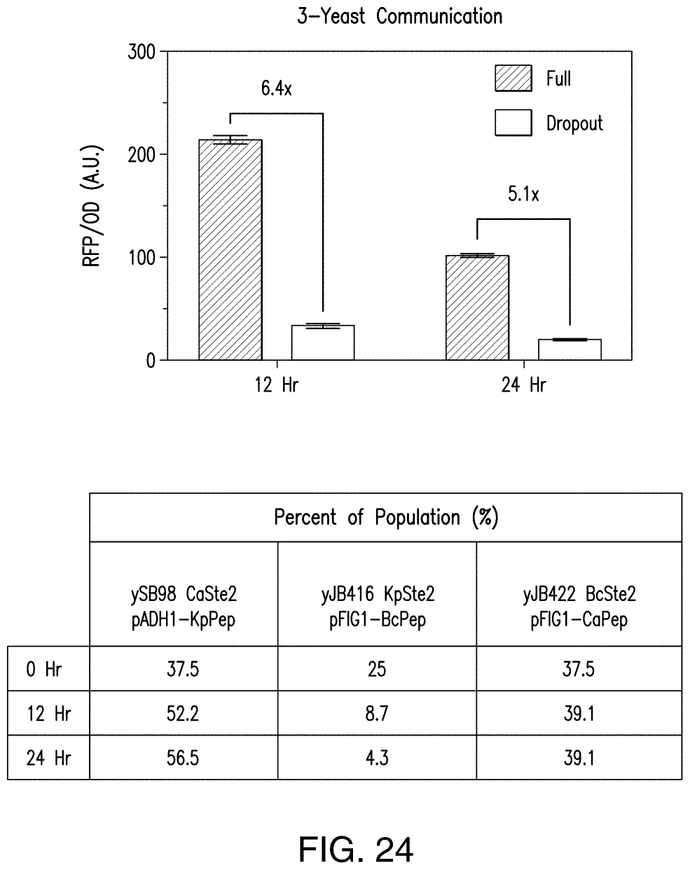

First, the established two-cell communication links were combined into a scalable paracrine ring topology. A ring is a network topology in which each cell cx connects to exactly two other cells (cx−1 and cx+1), forming a single continuous signal flow. The ring topology can be efficiently scaled by adding additional links. Failure of one of the links in the ring leads to complete interruption of information flow, allowing simultaneous monitoring of the functionality and continued presence of all ring members. The two-cell links were combined into rings of increasing size, from two to six members (FIG. 18C, topologies 1-6). Information flow was started by cell c1 constitutively secreting the peptide sensed by cell c2 through GPCR g2. Peptide sensing in cell c2 was coupled to secretion of peptide p3 sensed by cell c3 through GPCR g3. In this manner, peptide signals were transmitted around the ring. The N-member ring is closed by cell cN secreting the peptide sensed by cell c1 through GPCR g1. c1 reports on ring closure by a GPCR-coupled fluorescence read-out (FIG. 21). This was started with assembling a two- and a three-member ring (FIG. 18D and FIG. 22). An interrupted ring, with one member dropped out, was used as a control and the results are reported as fold-change in fluorescence between the full-ring and the interrupted ring. Colony PCR was used to assess the culture composition over time in the three-member ring. Due to differential growth behavior of individual strains (FIG. 23), it was observed that single strains eventually took over the culture (FIG. 24).

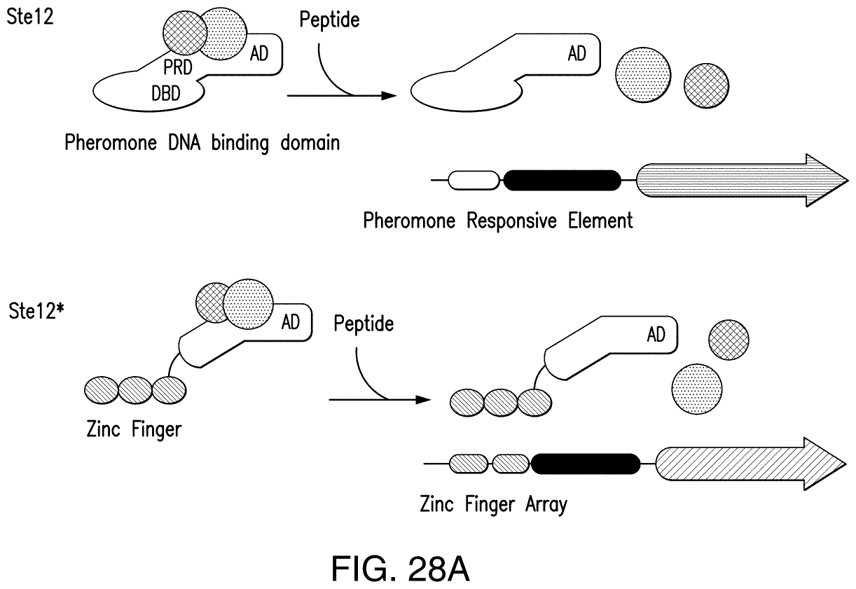

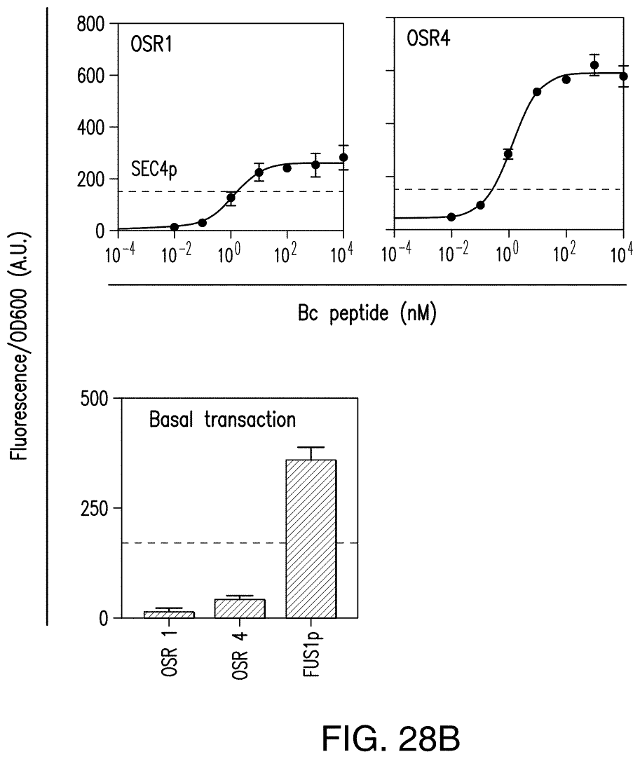

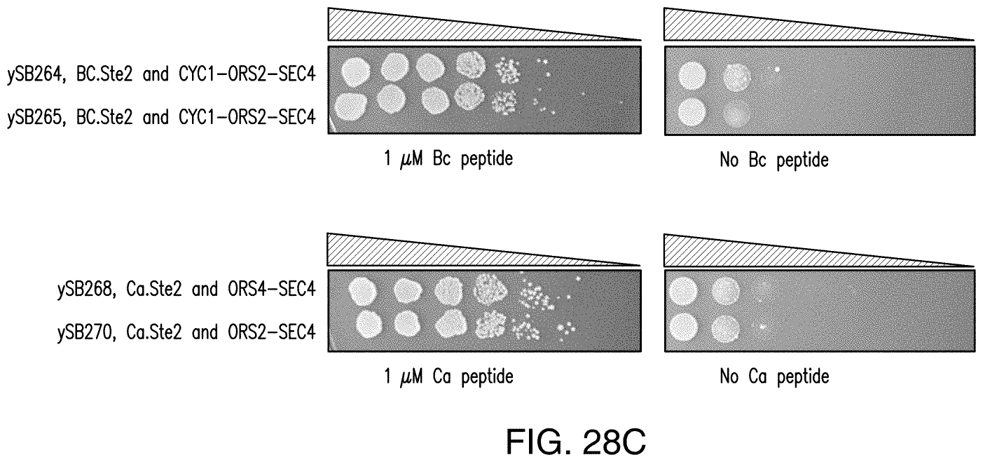

The differential growth phenotypes were partly caused by the expression and secretion burden of specific combinations of GPCRs and peptides. This can be addressed by improving expression and secretion levels. Growth phenotypes were also caused by GPCR-activation (and downstream activation of the mating response) and can be alleviated by using an orthogonal Ste12* that decouples GPCR-activation from the mating response (FIG. 28).

Next, in order to test for inherent scalability, the number of members in the communication ring was increased stepwise from three to six members (FIG. 18D and FIG. 22).

To test if a different interconnected communication topology can be achieved, a branched tree topology using cells co-expressing two GPCRs and accordingly being able to process two inputs (dual-input nodes) was also implemented. Such topologies allow integration of multiple information inputs and report on the presence of at least one of these distributed inputs. Functional signal flow was first tested through a three-yeast linear bus topology able to process two inputs (FIG. 18C, topology 6). Then, two branches upstream of the three-yeast bus and a side branch eventually leading to a six-yeast tree with two dual-input nodes were then added (FIG. 18C, topology 7 and FIGS. 25 and 26). To test functionality of communication, the information flow was started by adding the synthetic peptide ligand(s) recognized by the yeast cells starting each branch (single, dual and triple inputs were compared) (FIGS. 18E and F). Only the last yeast cell encoded a peptide-controlled fluorescent readout, enabling measurement once information traveled successfully through the topology by comparing the fold change in fluorescence compared with not adding starting peptide.

### Example 5. The Synthetic Communication Language Enables Construction of an Interdependent Microbial Community

Next, to anticipate a real application of the language, its orthogonal interfaces were leveraged to render yeast cells mutually dependent based on peptide signaling and essential gene activation.

Engineered interdependence is of central importance for synthetic ecology as the integrity of synthetic consortia can be enforced. Certain current approaches to engineer mutual dependence in synthetic communities rely on metabolite cross feeding⁵⁰, which limits the number of members that can be rapidly added to such a microbial community, and can suffer from a dependence on cross feeding metabolically expensive molecules needed at substantial molar concentrations. The peptide signal-based interdependence is conceptually different from cross feeding metabolites as interfaces that are orthogonal to the cellular metabolism were used, that allow scaling the number of community members by peptide/GPCR gene swapping and which are sensitive enough to function at low nanomolar signal concentrations.

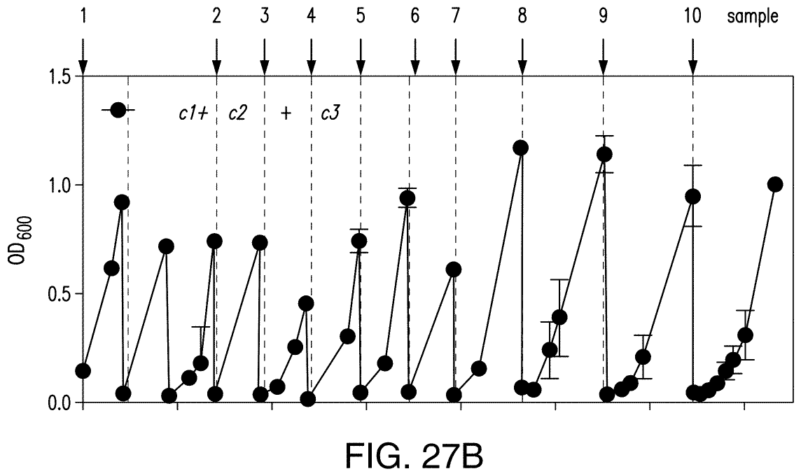

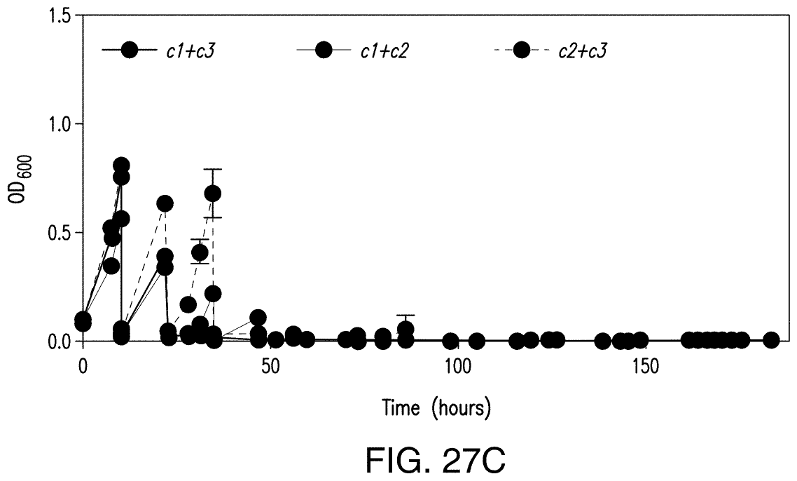

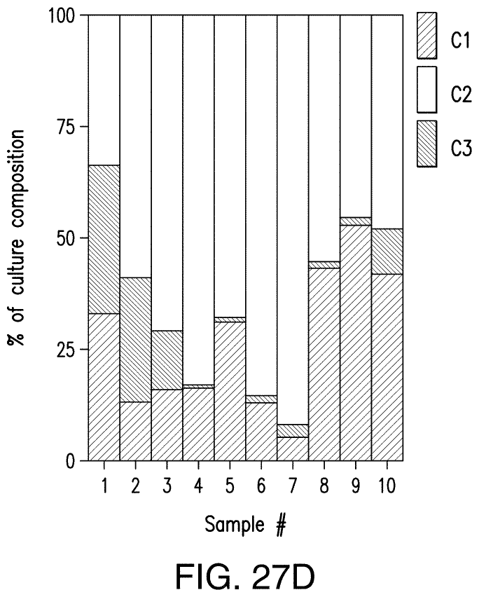

In order to engineer mutually dependent strain communities, an essential gene was placed under GPCR control (FIG. 27A). SEC4 was chosen as the target essential gene due to its performance in a previous study⁵³. An orthogonal Ste12* transcription factor and a set of tightly controlled orthogonal Ste12*-responsive promoters (OSR promoters) were engineered, matching the dynamic range to the expected intracellular SEC4 levels (FIG. 28A, FIG. 28B and FIG. 28C). The natural SEC4 promoter was replaced with one of the OSR promoters in strains expressing either the Bc.Ste2, Ca.Ste2 or Vp1.Ste2 receptors. FIG. 28A provides a schematic of the structure and function of an exemplary Ste12*. The natural pheromone-inducible transcription factor Ste12 is composed of a DNA binding domain (DBD), a pheromone-responsive domain (PRD) and an activation domain (AD) (see Pi, H. W., Chien, C. T. & Fields, S. Transcriptional activation upon pheromone stimulation mediated by a small domain of Saccharomyces cerevisiae Ste12p. Mol Cell Biol 17, 6410-6418 (1997)). The orthogonal Ste12* was engineered by replacing the DBD by the zinc-finger-based DNA binding domain 43-8 (see Khalil, A. S. et al. A Synthetic Biology Framework for Programming Eukaryotic Transcription Functions. Cell 150, 647-658 (2012)). The Ste12* binds to a zinc-finger responsive element (ZFRE) in a given synthetic promoter. It does not recognize the natural pheromone response element anymore that the Ste12 binds to. The lower panel of FIG. 28B, highlights the basal transcription levels from the OSR1 and OSR4 promoters in the absence of plasmid, which are compared to the basal transcription levels of the FUS1 promoter, which is relatively leaky. Designed orthogonal ste12*-responsive promoters (OSR promoters) feature a core promoter with an 8× repetitive ZFRE upstream of it, and OSR1 features a CYC1t core promoter with an integrated upstream repressor element (URS) (see Vidal, M., Brachmann, R. K., Fattaey, A., Harlow, E. & Boeke, J. D. Reverse two-hybrid and one-hybrid systems to detect dissociation of protein-protein and DNA-protein interactions. Proceedings of the National Academy of Sciences of the United States of America 93, 10315-10320 (1996)) to reduce basal transcription. OSR4 features the synthetic core promoter 2 (see Redden, H. & Alper, H. S. The development and characterization of synthetic minimal yeast promoters. Nature communications 6, 7810 (2015)).

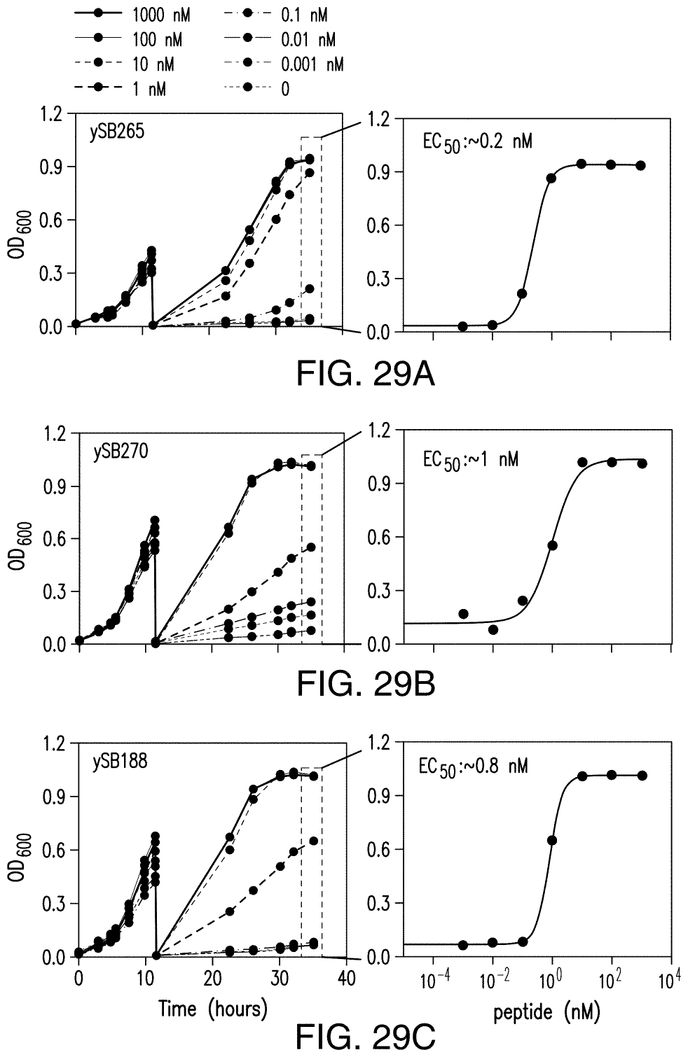

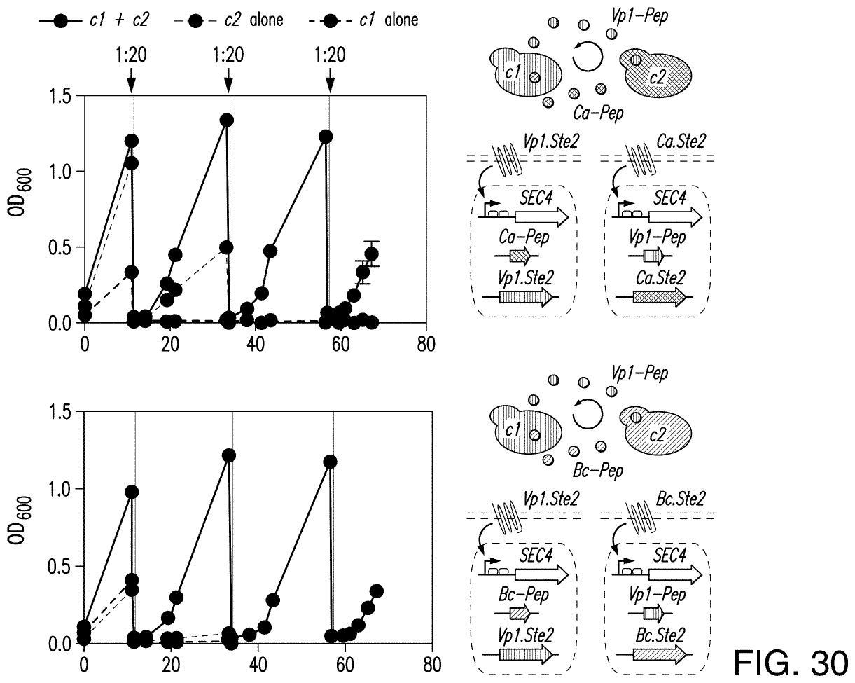

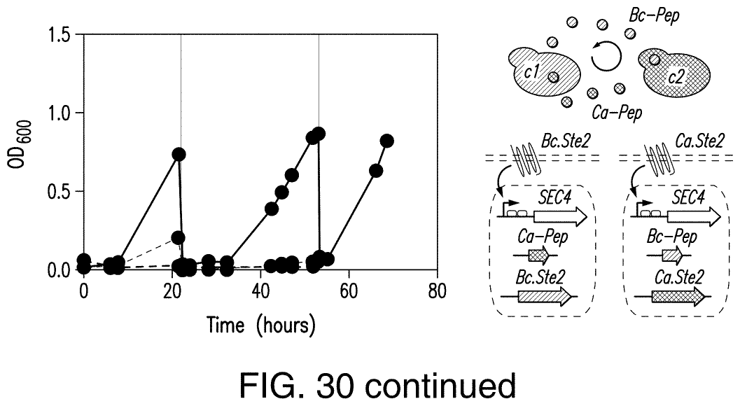

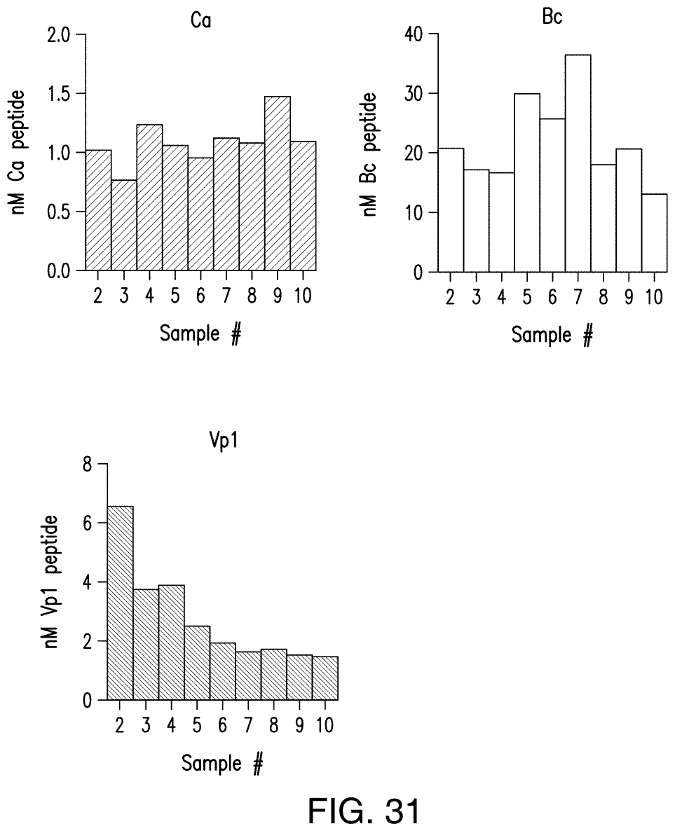

As expected, the resulting strains were dependent on peptide for growth and showed peptide/growth EC₅₀ values in the nanomolar range, which was achievable by secretion (FIG. 29). All strains were transformed with either of the two non-cognate constitutive peptide expression plasmids. The resulting six strains were used to assemble all three combinations of interdependent two-member links and their growth in strict mutual dependence over >60 hours (>15 doublings) was verified (FIG. 30). The growth rate of the two-membered consortium was thereby dependent on the member identity, probably defined by the secreted amount of a given peptide and the dose response characteristics of a given GPCR. The interdependent community was then scaled to three members and stable mutually dependent growth of this three-member cycle over >7 days (>50 doublings) was demonstrated, while communities missing one essential member collapsed (FIG. 27B-C). The presence of each strain and peptide over time was verified (FIG. 27D and FIG. 31). Stable ratios of community members were not reached over the course of this experiment, suggesting that scaling in the number of members elicits more complex community behaviors. Mathematical modeling as well as experimental parameterization of peptide secretion rates and peptide-secretion-linked growth rates can be used to understand and harness these interesting dynamics. Once predictable, “peptide-signal interdependence” will allow fine-tuning the abundance of each strain in a consortium eventually allowing one to control abundance in space and time.

In summary, fungal mating peptide/GPCR pairs were repurposed into a scalable language with an extensible number of orthogonal interfaces—unique channels are one of the current bottlenecks in scaling the complexity of synthetic ecology communities.

The fungal pheromone response pathway constitutes an ideal source for a large pool of unique signal and receiver interfaces that can be harnessed to build this modular, synthetic communication language.

These interfaces are accessible by genome mining as both the peptides and the GPCRs are genetically encoded and can be implemented by simple gene cloning.

Genome mining alone yields a high number of off-the-shelf orthogonal interfaces whose component diversity can potentially be further scaled and tuned by directed evolution to exploit the full information density of 9-13 amino acid peptide ligands (sequence space >10¹⁴). Further, the language can be tuned by ligand recoding, as small changes in the sequence of a given peptide ligand alters the response behavior of a given GPCR. Importantly, changing the ligand sequence can be achieved by simple cloning and does not require receptor or metabolic engineering. In addition, peptides are technically ideal as a signal. Peptides are stable and rich in molecular information and virtually any short peptide sequence is readily available through commercial solid-phase synthesis allowing for the rapid characterization and evolution of new peptide-sensing mating GPCRs.

The peptide/GPCR language is modular and insulated, and thus likely portable to many other Ascomycete fungi as this is where the component modules are derived. Furthermore, as has been done for mammalian GPCRs in yeast, this system can be portable to animal and plant cells. Its simplicity suggests that the system will be easy for other laboratories to adopt, scale and customize, especially in the light of new tools for the rational tuning of GPCR-signaling in yeast.⁵⁴

The language is compatible with existing and future synthetic biology tools for applications such as biosensing, biomanufacturing^{55,56} or building living computers^{41,57}.

The disclosure of S. Billerbeck et al. (2018) Nature Communications volume 9, Article number: 5057, published Nov. 28, 2018, is incorporated by reference herein in its entirety.

### REFERENCES

1. Maharbiz, M. M. Synthetic multicellularity. Trends in cell biology 22, 617-623 (2012).
2. Teague, B. P., Guye, P. & Weiss, R. Synthetic Morphogenesis. Cold Spring Harbor perspectives in biology 8 (2016).
3. Wang, H. H., Mee, M. T. & Church, G. M. Applications of Engineered Synthetic Ecosystems. Synthetic Biology: Tools and Applications, 317-325 (2013).
4. Szathmary, E. & Smith, J. M. The Major Evolutionary Transitions. Nature 374, 227-232 (1995).
5. Rokas, A. The Origins of Multicellularity and the Early History of the Genetic Toolkit For Animal Development. Annu Rev Genet 42, 235-251 (2008).
6. Davies, D. G. et al. The involvement of cell-to-cell signals in the development of a bacterial biofilm. Science 280, 295-298 (1998).
7. Hammer, B. K. & Bassler, B. L. Quorum sensing controls biofilm formation in Vibrio cholerae. Mol Microbiol 50, 101-114 (2003).
8. Sperandio, V., Torres, A. G., Jarvis, B., Nataro, J. P. & Kaper, J. B. Bacteria-host communication: The language of hormones. Proceedings of the National Academy of Sciences of the United States of America 100, 8951-8956 (2003).
9. Elias, S. & Banin, E. Multi-species biofilms: living with friendly neighbors. Fems Microbiol Rev 36, 990-1004 (2012).
10. Clevers, H., Loh, K. M. & Nusse, R. An integral program for tissue renewal and regeneration: Wnt signaling and stem cell control. Science 346, 54-+ (2014).
11. Laughlin, S. B. & Sejnowski, T. J. Communication in neuronal networks. Science 301, 1870-1874 (2003).
12. Waters, C. M. & Bassler, B. L. Quorum sensing: Cell-to-cell communication in bacteria. Annu Rev Cell Dev Bi 21, 319-346 (2005).
13. Nealson, K. H., Platt, T. & Hastings, J. W. Cellular control of the synthesis and activity of the bacterial luminescent system. Journal of bacteriology 104, 313-322 (1970).
14. Basu, S., Gerchman, Y., Collins, C. H., Arnold, F. H. & Weiss, R. A synthetic multicellular system for programmed pattern formation. Nature 434, 1130-1134 (2005).
15. Kobayashi, H. et al. Programmable cells: Interfacing natural and engineered gene networks. Proceedings of the National Academy of Sciences of the United States of America 101, 8414-8419 (2004).
16. Tamsir, A., Tabor, J. J. & Voigt, C. A. Robust multicellular computing using genetically encoded NOR gates and chemical ‘wires’. Nature 469, 212-215 (2011).
17. You, L., Cox, R. S., 3rd, Weiss, R. & Arnold, F. H. Programmed population control by cell-cell communication and regulated killing. Nature 428, 868-871 (2004).
18. Din, M. O. et al. Synchronized cycles of bacterial lysis for in vivo delivery. Nature 536, 81-+(2016).
19. Chen, Y., Kim, J. K., Hirning, A. J., Josic, K. & Bennett, M. R. SYNTHETIC BIOLOGY. Emergent genetic oscillations in a synthetic microbial consortium. Science 349, 986-989 (2015).
20. You, Y. S. et al. Use of bacterial quorum-sensing components to regulate gene expression in plants. Plant Physiol 140, 1205-1212 (2006).
21. Neddermann, P. et al. A novel, inducible, eukaryotic gene expression system based on the quorum-sensing transcription factor TraR (vol 4, pg 159, 2003). Embo Rep 4, 439-439 (2003).
22. Abisado, R. G., Benomar, S., Klaus, J. R., Dandekar, A. A. & Chandler, J. R. Bacterial Quorum Sensing and Microbial Community Interactions. Mbio 9 (2018).
23. Canton, B., Labno, A. & Endy, D. Refinement and standardization of synthetic biological parts and devices. Nat Biotechnol 26, 787-793 (2008).
24. Davis, R. M., Muller, R. Y. & Haynes, K. A. Can the natural diversity of quorum-sensing advance synthetic biology? Frontiers in bioengineering and biotechnology 3, 30 (2015).
25. Collins, C. H., Leadbetter, J. R. & Arnold, F. H. Dual selection enhances the signaling specificity of a variant of the quorum-sensing transcriptional activator LuxR (vol 24, pg 708, 2006). Nat Biotechnol 24, 1033-1033 (2006).
26. Scott, S. R. & Hasty, J. Quorum Sensing Communication Modules for Microbial Consortia. ACS synthetic biology 5, 969-977 (2016).
27. Marchand, N. & Collins, C. H. Synthetic Quorum Sensing and Cell-Cell Communication in Gram-Positive Bacillus megaterium. ACS synthetic biology 5, 597-606 (2016).
28. Gamby, S. et al. Altering the Communication Networks of Multispecies Microbial Systems Using a Diverse Toolbox of AI-2 Analogues. Acs Chem Biol 7, 1023-1030 (2012).
29. Ji, G. Y., Beavis, R. & Novick, R. P. Bacterial interference caused by autoinducing peptide variants. Science 276, 2027-2030 (1997).
30. Schauder, S., Shokat, K., Surette, M. G. & Bassler, B. L. The LuxS family of bacterial autoinducers: biosynthesis of a novel quorum-sensing signal molecule. Mol Microbiol 41, 463-476 (2001).
31. Roy, V., Adams, B. L. & Bentley, W. E. Developing next generation antimicrobials by intercepting AI-2 mediated quorum sensing. Enzyme Microb Tech 49, 113-123 (2011).
32. Xavier, K. B. & Bassler, B. L. Interference with AI-2-mediated bacterial cell-cell communication. Nature 437, 750-753 (2005).
33. Adams, B. L. et al. Evolved Quorum Sensing Regulator, LsrR, for Altered Switching Functions. ACS synthetic biology 3, 210-219 (2014).
34. Hauk, P. et al. Insightful directed evolution of Escherichia coli quorum sensing promoter region of the lsrACDBFG operon: a tool for synthetic biology systems and protein expression. Nucleic Acids Res 44, 10515-10525 (2016).
35. Morsut, L. et al. Engineering Customized Cell Sensing and Response Behaviors Using Synthetic Notch Receptors. Cell 164, 780-791 (2016).
36. Ostrov, N. et al. A modular yeast biosensor for low-cost point-of-care pathogen detection. Science advances 3, e1603221 (2017).
37. Jones, S. K. & Bennett, R. J. Fungal mating pheromones: Choreographing the dating game. Fungal Genet Biol 48, 668-676 (2011).
38. Xue, C. Y., Hsueh, Y. P. & Heitman, J. Magnificent seven: roles of G protein-coupled receptors in extracellular sensing in fungi. Fems Microbiol Rev 32, 1010-1032 (2008).
39. Hennig, S., Clemens, A., Rodel, G. & Ostermann, K. A yeast pheromone-based inter-species communication system. Appl Microbiol Biot 99, 1299-1308 (2015).
40. Youk, H. & Lim, W. A. Secreting and Sensing the Same Molecule Allows Cells to Achieve Versatile Social Behaviors. Science 343, 628-+(2014).
41. Regot, S. et al. Distributed biological computation with multicellular engineered networks. Nature 469, 207-211 (2011).
42. Martin, N. P., Celic, A. & Dumont, M. E. Mutagenic mapping of helical structures in the transmembrane segments of the yeast alpha-factor receptor. J Mol Biol 317, 765-788 (2002).
43. Celic, A. et al. Sequences in the intracellular loops of the yeast pheromone receptor Ste2p required for G protein activation. Biochemistry 42, 3004-3017 (2003).
44. Keppler-Ross, S., Noffz, C. & Dean, N. A new purple fluorescent color marker for genetic studies in Saccharomyces cerevisiae and Candida albicans. Genetics 179, 705-710 (2008).
45. Kipniss, N. H. et al. Engineering cell sensing and responses using a GPCR-coupled CRISPR-Cas system. Nature communications 8 (2017).
46. Mukherjee K., B. S., Peralta-Yahya, P. GPCR-based chemical sensors for medium-chain fatty acids. ACS synthetic biology 4, 1261 (2015).
47. Manfredi, J. P. et al. Yeast alpha mating factor structure-activity relationship derived from genetically selected peptide agonists and antagonists of Ste2p. Molecular and cellular biology 16, 4700-4709 (1996).
48. Awan, A. R. et al. Biosynthesis of the antibiotic nonribosomal peptide penicillin in baker's yeast. Nature communications 8 (2017).
49. Villarreal, F. et al. Synthetic microbial consortia enable rapid assembly of pure translation machinery. Nat Chem Biol 14, 29-+(2018).
50. Johns, N. I., Blazejewski, T., Gomes, A. L. & Wang, H. H. Principles for designing synthetic microbial communities. Current opinion in microbiology 31, 146-153 (2016).
51. Liu, Z. et al. Engineering of a novel cellulose-adherent cellulolytic Saccharomyces cerevisiae for cellulosic biofuel production. Sci Rep-Uk 6 (2016).
52. Austin, H. P. et al. Characterization and engineering of a plastic-degrading aromatic polyesterase. Proceedings of the National Academy of Sciences of the United States of America (2018).
53. Agmon, N. et al. Low escape-rate genome safeguards with minimal molecular perturbation of Saccharomyces cerevisiae. Proceedings of the National Academy of Sciences of the United States of America 114, E1470-E1479 (2017).
54. Shaw, W. et al. Engineering a model cell for rational tuning of GPCR signaling. bioRxiv 390559; doi: https://doi.org/10.1101/390559 (2018).
55. Ro, D. K. et al. Production of the antimalarial drug precursor artemisinic acid in engineered yeast. Nature 440, 940-943 (2006).
56. Galanie, S., Thodey, K., Trenchard, I. J., Filsinger Interrante, M. & Smolke, C. D. Complete biosynthesis of opioids in yeast. Science 349, 1095-1100 (2015).
57. Urrios, A. et al. A Synthetic Multicellular Memory Device. ACS synthetic biology 5, 862-873 (2016).
58. Brachmann, C. B. et al. Designer deletion strains derived from Saccharomyces cerevisiae S288C: a useful set of strains and plasmids for PCR-mediated gene disruption and other applications. Yeast 14, 115-132 (1998).
59. DiCarlo, J. E. et al. Genome engineering in Saccharomyces cerevisiae using CRISPR-Cas systems. Nucleic Acids Res 41, 4336-4343 (2013).
60. Sherman, F. Getting started with yeast. Guide to Yeast Genetics and Molecular and Cell Biology, Pt B 350, 3-41 (2002).
61. Kaiser, C., Michaelis, S., Mitchell, A. & Cold Spring Harbor Laboratory. Methods in yeast genetics: a Cold Spring Harbor Laboratory course manual, Edn. 1994. (Cold Spring Harbor Laboratory Press, Cold Spring Harbor, N.Y.; 1994).
62. Sherman, F. Getting started with yeast. Methods in enzymology 350, 3-41 (2002).
63. Mitchell, A. et al. The InterPro protein families database: the classification resource after 15 years. Nucleic Acids Res 43, D213-221 (2015).
64. Finn, R. D. et al. Pfam: the protein families database. Nucleic Acids Res 42, D222-230 (2014).
65. Sievers, F. et al. Fast, scalable generation of high-quality protein multiple sequence alignments using Clustal Omega. Mol Syst Biol 7 (2011).
66. Martin, S. H., Wingfield, B. D., Wingfield, M. J. & Steenkamp, E. T. Causes and Consequences of Variability in Peptide Mating Pheromones of Ascomycete Fungi. Mol Biol Evol 28, 1987-2003 (2011).
67. Gibson, D. G. et al. Enzymatic assembly of DNA molecules up to several hundred kilobases. Nat Methods 6, 343-U341 (2009).
68. Agmon, N. et al. Yeast Golden Gate (yGG) for efficient assembly of S. cerevisiae transcription units. ACS synthetic biology (2015).
69. DiCarlo, J. E. et al. Genome engineering in Saccharomyces cerevisiae using CRISPR-Cas systems. Nucleic acids research 41, 4336-4343 (2013).
70. Farzadfard, F., Perli, S. D. & Lu, T. K. Tunable and Multifunctional Eukaryotic Transcription Factors Based on CRISPR/Cas. ACS synthetic biology 2, 604-613 (2013).
71. Khalil, A. S. et al. A Synthetic Biology Framework for Programming Eukaryotic Transcription Functions. Cell 150, 647-658 (2012).

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

The contents of all figures and all references, patents and published patent applications and Accession numbers cited throughout this application are expressly incorporated herein by reference.

1. A genetically-engineered cell expressing:
(a) at least one heterologous G-protein coupled receptor (GPCR), wherein the amino acid sequence of the heterologous GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211; and/or
(b) at least one heterologous secretable GPCR peptide ligand, wherein the amino acid sequence of the heterologous GPCR peptide ligand is at least about 75% homologous to an amino acid sequence comprising any one of SEQ ID NOs: 1-116 or an amino acid sequence provided in Table 12 and/or encoded by a nucleotide sequence that is about 75% homologous to a nucleotide sequence comprising any one of SEQ ID NOs: 215-230.

2. The genetically-engineered cell of claim 1, wherein the heterologous GPCR is selectively activated by a ligand.

3. The genetically-engineered cell of claim 2, wherein the ligand is selected from the group consisting of peptide, a protein or portion thereof, a toxin, a small molecule, a nucleotide, a lipid, a chemical, a photon, an electrical signal and a compound.

4. The genetically-engineered cell of claim 2, wherein the ligand comprises an amino acid sequence that is at least about 75% homologous to an amino acid sequence of any one of SEQ ID NOs: 1-116 or an amino acid sequence provided in Table 12 and/or encoded by a nucleotide sequence that is about 75% homologous to a nucleotide sequence comprising any one of SEQ ID NOs: 215-230.

5. The genetically-engineered cell of claim 1, wherein the genetically-engineered cell is:
(a) a fungal cell;
(b) a fungal cell from the phylum Ascomycota; and/or
(c) a fungal cell selected from the group consisting of Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailiff, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, Capronia coronate and combinations thereof.

6. An intercellular signaling system comprising two or more, three or more, four or more or five or more genetically-engineered cells of claim 1.

7. An intercellular signaling system comprising:
(i) (a) a first genetically-engineered cell expressing at least one secretable G-protein coupled receptor (GPCR) ligand; and (b) a second genetically-engineered cell expressing at least one heterologous GPCR, wherein (i) the amino acid sequence of the heterologous GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211 and/or (ii) the amino acid sequence of the secretable GPCR ligand is at least about 75% homologous to an amino acid sequence comprising any one of SEQ ID NOs: 1-116 or an amino acid sequence provided in Table 12 and/or is encoded by a nucleotide sequence that is about 75% homologous to a nucleotide sequence comprising any one of SEQ ID NOs: 215-230; or
(ii) (a) a first genetically-engineered cell comprising: (i) a nucleic acid encoding a first heterologous G-protein coupled receptor (GPCR); and/or (ii) a nucleic acid encoding a first secretable GPCR ligand; and (b) a second genetically-engineered cell comprising: (i) a nucleic acid encoding a second heterologous GPCR; and/or (ii) a nucleic acid encoding a second secretable GPCR ligand, wherein (i) the first GPCR and/or the second GPCR is at least about 75% homologous to an amino acid sequence comprising any one of SEQ ID NOs: 117-161 or an amino acid sequence provided in Table 11 and/or is encoded by a nucleotide sequence that is at least about 75% homologous to a nucleotide sequence comprising any one of SEQ ID NOs: 168-211; and/or (ii) the first and/or second secretable GPCR peptide ligand is at least about 75% homologous to an amino acid sequence comprising any one of SEQ ID NOs: 1-116 or an amino acid sequence provided in Table 12 and/or is encoded by a nucleotide sequence that is about 75% homologous to a nucleotide sequence comprising any one of SEQ ID NOs: 215-230.

8. The intercellular signaling system of claim 7, wherein (i) the secretable GPCR ligand and/or the heterologous GPCR is identified and/or derived from a eukaryotic organism and/or (ii) the heterologous GPCR is activated by an exogenous ligand.

9. The intercellular signaling system of claim 7, wherein (i) the secretable GPCR ligand of the first genetically-engineered cell selectively activates the heterologous GPCR of the second genetically-engineered cell and/or (ii) the secretable GPCR ligand of the first genetically-engineered cell does not activate the heterologous GPCR of the second genetically-engineered cell.

10. The intercellular signaling system of claim 7, wherein the second genetically-engineered cell further expresses at least one secretable GPCR ligand and/or the first genetically-engineered cell further expresses at least one heterologous GPCR.

11. The intercellular signaling system of claim 10, wherein:
(a) the secretable GPCR ligand expressed by the second genetically-engineered cell is different from the secretable GPCR ligand expressed by the first genetically-engineered cell, e.g., selectively activate different GPCRs;
(b) the secretable GPCR ligand expressed by the second genetically-engineered cell does not activate the heterologous GPCR expressed by the second genetically-engineered cell;
(c) the heterologous GPCR expressed by the first genetically-engineered cell is different from the heterologous GPCR expressed by the second genetically-engineered cell, e.g., are selectively activated by different ligands;
(d) the secretable GPCR ligand expressed by the first genetically-engineered cell does not activate the heterologous GPCR expressed by the first genetically-engineered cell;
(e) the secretable GPCR ligand of the first genetically-engineered cell selectively activates the heterologous GPCR of the second genetically-engineered cell;
(f) the secretable GPCR ligand of the first genetically-engineered cell does not activate the heterologous GPCR of the second genetically-engineered cell;
(g) the secretable GPCR ligand expressed by the second genetically-engineered cell selectively activates the heterologous GPCR expressed by the first genetically-engineered cell;
(h) the secretable GPCR ligand expressed by the second genetically-engineered cell does not activate the heterologous GPCR expressed by the first genetically-engineered cell; and/or
(i) the secretable GPCR ligand expressed by the second genetically-engineered cell and/or the first genetically-engineered cell selectively activates a GPCR expressed by a third cell.

12. The intercellular signaling system of claim 7, wherein:
(a) one or more endogenous GPCR genes of the first genetically-engineered cell and/or the second genetically-engineered cell are knocked out;
(b) one or more endogenous GPCR ligand genes of the first genetically-engineered cell and/or the second genetically-engineered cell are knocked out;
(c) the first genetically-engineered cell and/or the second genetically-engineered cell further comprises a nucleic acid that encodes a product of interest;
(d) the first genetically-engineered cell and/or the second genetically-engineered cell further comprises a nucleic acid that encodes a sensor; and/or
(e) the first genetically-engineered cell and/or the second genetically-engineered cell further comprises a nucleic acid that encodes a detectable reporter.

13. The intercellular signaling system of claim 12, wherein the product of interest is selected from the group consisting of hormones, toxins, receptors, fusion proteins, regulatory factors, growth factors, complement system factors, enzymes, clotting factors, anti-clotting factors, kinases, cytokines, CD proteins, interleukins, therapeutic proteins, diagnostic proteins, biosynthetic pathways, antibodies and combinations thereof.

14. The intercellular signaling system of claim 7 further comprising:
(a) a third genetically-engineered cell;
(b) a third genetically-engineered cell and a fourth genetically-engineered cell;
(c) a third genetically-engineered, a fourth genetically-engineered cell and a fifth genetically-engineered cell;
(d) a third genetically-engineered, a fourth genetically-engineered cell, a fifth genetically-engineered cell and a sixth genetically-engineered cell;
(e) a third genetically-engineered, a fourth genetically-engineered cell, a fifth genetically-engineered cell, a sixth genetically-engineered cell and a seventh genetically-engineered cell; or
(f) a third genetically-engineered, a fourth genetically-engineered cell, a fifth genetically-engineered cell, a sixth genetically-engineered cell, a seventh genetically-engineered cell and an eighth genetically-engineered cell or more,
wherein each genetically-engineered cell expresses at least one heterologous GPCR and/or at least one secretable GPCR ligand,
wherein (i) each of the heterologous GPCRs are different, e.g., are selectively activated by different ligands, and/or each of the secretable GPCR ligands are different, e.g., selectively activate different GPCRs and/or (ii) one or more heterologous GPCRs are the same and/or one or more of the secretable GPCR ligands are the same.

15. The intercellular signaling system of claim 14, wherein the intercellular signaling system comprises a topology selected from the group consisting of a daisy chain network topology, a bus type network topology, a branched type network topology, a ring network topology, a mesh network topology, a hybrid network topology, a star type network topology and a combination thereof.

16. A kit comprising the genetically-engineered cell of claim 1.

17. A kit comprising the intercellular signaling system of claim 7.

18. A method of using the intercellular signaling system of claim 7:
(a) for spatial control of gene expression and/or temporal control of gene expression;
(b) for the generation of pharmaceuticals and/or therapeutics;
(c) for performing computations;
(d) as a biosensor; and/or
(e) for the generation of a product of interest.

19. A method for the identification of a G-protein coupled receptor (GPCR) and/or a GPCR ligand to be expressed in a genetically-engineered cell, comprising:
(a) searching a protein and/or genomic database and/or literature for a protein and/or a gene with homology to: (i) a S. cerevisiae Ste2 receptor and/or Ste3 receptor; (ii) a GPCR comprising an amino acid sequence comprising any one of SEQ ID NOs: 117-161; (iii) a GPCR comprising an amino acid sequence provided in Table 11; and/or (iv) a GPCR encoded by a nucleotide sequence comprising any one of SEQ ID NOs: 168-211 to identify a GPCR; and/or
(b) searching a protein and/or genomic database and/or literature for a protein, peptide and/or a gene with homology to: (i) a GPCR peptide ligand comprising an amino acid sequence comprising any one of SEQ ID NOs: 1-116; (ii) a GPCR peptide ligand comprising an amino acid sequence provided in Table 12; (iii) a GPCR peptide ligand encoded by a nucleotide sequence comprising any one of SEQ ID NOs: 215-230 to identify a GPCR ligand; and/or (iv) a yeast pheromone or a motif thereof.

20. A genetically-engineered cell expressing a GPCR and/or GPCR ligand identified by the method of claim 19.


SEQUENCE LISTING (594 sequences)

SEQ ID NO: 1 - protein, 13 aa, Saccharomyces cerevisiae
WHWLQLKPGQPMY

SEQ ID NO: 2 - protein, 14 aa, Saccharomyces castellii
NWHWLRLDPGQPLY

SEQ ID NO: 3 - protein, 13 aa, Vanderwaltozyma polyspora
WHWLRLRYGEPIY

SEQ ID NO: 4 - protein, 14 aa, Vanderwaltozyma polyspora
PWHWLRLRYGEPIY

SEQ ID NO: 5 - protein, 13 aa, Vanderwaltozyma polyspora
WHWLELDNGQPIY

SEQ ID NO: 6 - protein, 11 aa, Torulaspora delbrueckii
GWMRLRLGQPL

SEQ ID NO: 7 - protein, 11 aa, Torulaspora delbrueckii
GWMRLRLGQPM

SEQ ID NO: 8 - protein, 11 aa, Torulaspora delbrueckii
GWMRLRIGQPL

SEQ ID NO: 9 - protein, 13 aa, Saccharomyces kluyveri
WHWLSFSKGEPMY

SEQ ID NO: 10 - protein, 14 aa, Saccharomyces kluyveri
PWHWLSFSKGEPMY

SEQ ID NO: 11 - protein, 13 aa, Kluyveromyces lactis
WSWITLRPGQPIF

SEQ ID NO: 12 - protein, 15 aa, Kluyveromyces lactis
SPWSWITLRPGQPIF

SEQ ID NO: 13 - protein, 12 aa, Zygosaccharomyces rouxii
HFIELDPGQPMF

SEQ ID NO: 14 - protein, 13 aa, Zygosaccharomyces rouxii
AHFIELDPGQPMF

SEQ ID NO: 15 - protein, 12 aa, Zygosaccharomyces bailii
HLVRLSPGAAMF

SEQ ID NO: 16 - protein, 12 aa, Zygosaccharomyces bailii
PLVRLSPGAAMF

SEQ ID NO: 17 - protein, 13 aa, Zygosaccharomyces bailii
APLVRLSPGAAMF

SEQ ID NO: 18 - protein, 13 aa, Zygosaccharomyces bailii
AHLVRLSPGAAMF

SEQ ID NO: 19 - protein, 13 aa, Candida glabrata
WHWVRLRKGQGLF

SEQ ID NO: 20 - protein, 13 aa, Candida glabrata
WHWVKIRKGQGLF

SEQ ID NO: 21 - protein, 12 aa, Ashbya gossypii
WFRLSLHHGQSM

SEQ ID NO: 22 - protein, 12 aa, Scheffersomyces stipitis
WHWTSYGVFEPG

SEQ ID NO: 23 - protein, 13 aa, Scheffersomyces stipitis
PWHWTSYGVFEPG

SEQ ID NO: 24 - protein, 13 aa, Komagataella pastoris
FRWRNNEKNQPFG

SEQ ID NO: 25 - protein, 16 aa, Candida guilliermondii
KKNSRFLTYWFFQPIM

SEQ ID NO: 26 - protein, 13 aa, Candida parapsilosis
KPHWTTYGYYEPQ

SEQ ID NO: 27 - protein, 14 aa, Candida auris
KWGWLRFFPGEPFV

SEQ ID NO: 28 - protein, 14 aa, Yarrowia lipolytica
WRWFWLPGYGEPNW

SEQ ID NO: 29 - protein, 14 aa, Candida lusitaniae
KWKWIKFRNTDVIG

SEQ ID NO: 30 - protein, 13 aa, Candida lusitaniae
WGWIHFLNTDVIG

SEQ ID NO: 31 - protein, 15 aa, Candida lusitaniae
PKWKWIKFRNTDVIG

SEQ ID NO: 32 - protein, 13 aa, Candida albicans
GFRLTNFGYFEPG

SEQ ID NO: 33 - protein, 15 aa, Candida tropicalis
KFKFRLTRYGWFSPN

SEQ ID NO: 34 - protein, 13 aa, Candida tenuis
FSWNYRLKWQPIS

SEQ ID NO: 35 - protein, 12 aa, Lodderomyces elongisporous
WMWTRYGRFSPV

SEQ ID NO: 36 - protein, 15 aa, Lodderomyces elongisporous
DPGWMWTRYGRFSPV

SEQ ID NO: 37 - protein, 17 aa, Geotrichum candidum
GDWGWFWYVPRPGDPAM

SEQ ID NO: 38 - protein, 18 aa, Geotrichum candidum
PGDWGWFWYVPRPGDPAM

SEQ ID NO: 39 - protein, 13 aa, Baudoinia compniacensis
GWIGRCGVPGSSC

SEQ ID NO: 40 - protein, 23 aa, Schizosaccharomyces octosporus
TYEDFLRVYKNWWSFQNPDRPDL

SEQ ID NO: 41 - protein, 27 aa, Schizosaccharomyces octosporus
PACTTYEDFLRVYKNWWSFQNPDRPDL

SEQ ID NO: 42 - protein, 10 aa, Tuber melanosporum
WTPRPGRGAY

SEQ ID NO: 43 - protein, 9 aa, Aspergillus oryzae
WCALPGQGC

SEQ ID NO: 44 - protein, 23 aa, Schizosaccharomyces pombe
TYADFLRAYQSWNTFVNPDRPNL

SEQ ID NO: 45 - protein, 24 aa, Schizosaccharomyces pombe
KTYADFLRAYQSWNTFVNPDRPNL

SEQ ID NO: 46 - protein, 9 aa, Aspergillus fischeri
WCHLPGQGC

SEQ ID NO: 47 - protein, 10 aa, Pseudogymnoascus destructans
FCWRPGQPCG

SEQ ID NO: 48 - protein, 10 aa, Pseudogymnoascus destructans
FCQRPGQLCG

SEQ ID NO: 49 - protein, 12 aa, Pseudogymnoascus destructans
LEFGGLEKEQNS

SEQ ID NO: 50 - protein, 23 aa, Schizosaccharomyces japonicus
VSDRVKQMLSHWWNFRNPDTANL

SEQ ID NO: 51 - protein, 27 aa, Schizosaccharomyces japonicus
PERRVSDRVKQMLSHWWNFRNPDTANL

SEQ ID NO: 52 - protein, 9 aa, Paracoccidioides brasiliensis
WCTRPGQGC

SEQ ID NO: 53 - protein, 16 aa, Mycosphaerella graminicola
GNSFVGWCGAIGAPCA

SEQ ID NO: 54 - protein, 10 aa, Mycosphaerella graminicola
WCGAIGAPCA

SEQ ID NO: 55 - protein, 9 aa, Penicillium chrysogenum
WCGHIGQGC

SEQ ID NO: 56 - protein, 10 aa, Penicillium chrysogenum
KWCGHIGQGC

SEQ ID NO: 57 - protein, 10 aa, Aspergillus nidulans
WCRFRGQVCG

SEQ ID NO: 58 - protein, 15 aa, Phaeosphaeria nodorum
KYNGWRYRPYGLPVG

SEQ ID NO: 59 - protein, 10 aa, Hypocrea jecorina
WCYRIGEPCW

SEQ ID NO: 60 - protein, 10 aa, Hypocrea jecorina
WCWILGGKCW

SEQ ID NO: 61 - protein, 9 aa, Botrytis cinerea
WCGRPGQPC

SEQ ID NO: 62 - protein, 10 aa, Beauvaria bassiana
WCMRPGQPCW

SEQ ID NO: 63 - protein, 9 aa, Beauvaria bassiana
WCMQTPKCW

SEQ ID NO: 64 - protein, 11 aa, Neurospora crassa
QWCRIHGQSCW

SEQ ID NO: 65 - protein, 14 aa, Neurospora crassa
QVCNMRLHPKKVCW

SEQ ID NO: 66 - protein, 10 aa, Sporothrix scheckii
YCPLKGQSCW

SEQ ID NO: 67 - protein, 12 aa, Sporothrix scheckii
QRYCPLKGQSCW

SEQ ID NO: 68 - protein, 11 aa, Magnaporthe oryzea
QWCPRRGQPCW

SEQ ID NO: 69 - protein, 8 aa, Dactylellina haptotyla
WCVYNSCP

SEQ ID NO: 70 - protein, 10 aa, Fusarium graminearum
WCWWKGQPCW

SEQ ID NO: 71 - protein, 10 aa, Fusarium graminearum
WCTWKGQPCW

SEQ ID NO: 72 - protein, 14 aa, Capronia coronata
GLSYWKGVNDGGSS

SEQ ID NO: 73 - protein, 102 aa, Aspergillus fischeri
MRLLSLVLATFAATAVQADITPWCHLPGQGCYMLKRAADASDEVRRSASAVAEAVAEAFPQTPWCHLPGQGCAKAKRAAEAAEEVKRSADAFAEAMAAFEKE

SEQ ID NO: 74 - protein, 96 aa, Ashbya gossypii
MKTTHILSLATLAACAPVQPAPVQPTDLAAAANVPEKAVLGFFQLYNVGDVELLPVDDGAHSGILFVNRTLADVDYSSEHVVQKWFRLSLHHGQSM

SEQ ID NO: 75 - protein, 128 aa, Aspergillus nidulans
MKLFFVSILLAALLATAVKAAPAAELQHRWCRFAGRICPPTKRTADALNFVKREAEAVAEPFKINRWCRFRGQVCGKAKRAAEAIGNVKLSAEAVADAMAFLDELTREEYAQLAKDFGHLKESDNSDG

SEQ ID NO: 76 - protein, 103 aa, Aspergillus oryzae
MKLISVVVAALAATSVQAGVLQKWCSLPAQGCYMLKRAADASGDVRRSAEALSEAMPDAEALAKWCALPGQGCLKAKRAAEAVEEARRSADALADAMADLGEY

SEQ ID NO: 77 - protein, 193 aa, Beauvaria bassiana
MKLSLVMLATAATTVIAAPRPWCMRPGQPCWKLKRAVDALGEPAPSPVEPLDADNIGLFASGAHDRLLHLASSDAANVDDEGAFEKRWCMQTPKCWKLLADEDGELSKRWCMRPGQPCWKRSVDEHGDLAKRWCMRPGQPCWKAKRAAESVLNAGQEDGDAQEQDCGDDGECSVAKRHLDGLHHVARAIVEAF

SEQ ID NO: 78 - protein, 392 aa, Botrytis cinerea
MKFTNAIALAILAATATAVAVPEPWCGRPGQPCKREAVAVAAPVAEPWCGRPGQPCKRTPEAEAWCGRPGQPCKRDAEPWCGRPGQPCKREALPEAWCGRPGQPCKRTPLAEAEAEAWCGRPGQPCRKNKRAAEAVAEAFAEPWCGRPGQPCKRDAEADVSEAAIKRCNMVGGACFEAKRLARDLAEATAETVEDSDLFLRSLNIETREVSEVVAREAEAWCGRPGQPCKRDAEAWCGRPGQPCKREALAEAEAWCGRPGQPCKREALAEAEAWCGRPGQPCKRTAEPWCGRPGQPCKEKREADPEAEAWCGRPGQPCRAVKRAAEAIAEALAEPTAEAWCGRPGQPCKREALAEAEANAEAWCGRPGQPCRKAKRDAFALAYAADVALAQL

SEQ ID NO: 79 - protein, 397 aa, Baudoinia compniacensis
MKFSIVAVAAVAAQAAAVSGSTSAVFKDGVGACNVPGQKCHTVKNAARDILNAINKPTDVDDQQSYFCDIQGSAGCNQLHGSVDKLQQAAIKAYHTVAAREAEAEAEAEANPGYGWIGRCGVPGSSCNKKREADPGYGWIGRCGVPGSSCNKKRDEDAAAREHWLAQREAGGWIGRCGVPGSSCNKKREEEVEVLRREAEAGGWIGRCGVPGSSCNKARDANPGGWIGRCGVPGSSCNKKREAGGWIGRCGVPGSSCNKARDAEDDQKIQQMQDAIRAFNPEIEKAECNQDGQPCDLIKTAAQALHNNTRREAEAGGWIGRCGVPGSSCNKNKRALAFCQSGENCTGPAYAHLQSQDATADKAEKDCHGPNGACTIAARALAELEQAVDAALLDADA

SEQ ID NO: 80 - protein, 143 aa, Candida albicans
MKFSLTLLTATIATIVAAAPAQYTGQAIDSNQVVEIPESAVEAYFPIDDELTPVFGEIDNKPVILIVNGTTLTSGANNEKREAKSKGGFRLTNFGYFEPGKRDANADAGFRLTNFGYFEPGKRDANAEAGFRLTNFGYFEPGK

SEQ ID NO: 81 - protein, 217 aa, Candida auris
MKFSITAIIAATGSLVAAAPTPSSTDAPSFSEVPSSVESSFGVPTEAIIGQFSFDADEYPLLTVYEDRRYIILLNSTIMEEAYASLNSGNEKRDAEAEAKWGWLRFFPGEPFVKRDAEADAEAKWGWLRFFPGEPFVKRDAEADAEAKWGWLRFFPGEPFVKRDAEADAEAKWGWLRFFPGEPFVKRDADAEAKWGWLRFYPGEPFVKREVEADLEG

SEQ ID NO: 82 - protein, 225 aa, Capronia coronata
MHISSTTVTLVLTASFIQSALAFPVPAFLDVLRRDASPDPRLSYWKGVNDGGSSKIKSRRWLSPIIEMLDKREPGLSYWKGVNDGGSSKREAAPEPDPGLSYWKGVNDGGFSKREAEPEPEPEPRLPYWKGVNDGGSSKREAAPEPDPGLSYWKGVNDGGSSKREAAPEPEPEPEPGLSYWKGVNDGGSSKRGLSYWKGVNDGGSSKREAEPEPQPDALPALGLT

SEQ ID NO: 83 - protein, 159 aa, Candida glabrata
MRFLRFISTVALLITGLATAQPVGEELGETVEVPSEAFIGYLDFGATNDVAILPISNKTNNGLLFVNTTLYNQATKGEKLSDFTKRDANPDAEAEAWHWVKIRKGQGLFRRSADASPEAEAWHWVRLRKGQGLFRRSADASPEAEAWHWVRLRKGQGLF

SEQ ID NO: 84 - protein, 222 aa, Candida guilliermondii
MKFSTAFVSTLFATYAAAAPLAAASDKIPVPFPKSAVNQIVTIDETNAPIYLNNSGTITLFLVNTTVKEESPEKRELGEVATGYEFNAAQYMKRESFPIENLVPESSLEKREDKKNSRFLTYWFFQPIMKRGEEETSEVVKREAKKNSRFLTYWFFQPIMKREEDIVAGDEMVKREAKKNSRFLTYWFFQPIMKREGGNEVEKRDAKKNSRFLTYWFFQPIM

SEQ ID NO: 85 - protein, 228 aa, Candida lusitaniae
MKFSLAIIFSLAAAVVSAAPVAPESSSDFQIPEEAIISSQALGDDQLPLLLGEGNATYFVLVNGTTLAEAYGITKRDAEAFDATYLGSSVAKREANADAWGWIHFLNTDVIGKRDAEPKWKWIHFRNTDVIGKRDASPKWKWIKFRNTDVIGKRDAEADASPKWKWIKFRNTDVIGKRDAEADAAPKWKWIKFRNTDVIGKRDANAAPKWRWINFRNTDVIGKREAQE

SEQ ID NO: 86 - protein, 145 aa, Candida tenuis
MRLSTILTLALTSKFVFSAPVEKVKREDGLDVPDEAIIAVYPIDEYKQPFYAEADGQNYVVILNTTALGEADLAKRDADAFSWNYRLKWQPISKRDADADADADAFSWNYRLKWQPISKRDADADADADADAFSWNYRLKWQPIS

SEQ ID NO: 87 - protein, 135 aa, Candida parapsilosis
MKFSIAVLTAIAAALVASAPVASKEAEVPALPVDNVLERVVEAFFNGPSIDAEIKDKTAADVKGVVGSQKREAEAKPHWTTYGYYEPQKRDANAEAEAKPHWTTYGYYEPQKRDANAEAEAKPHWTTYGYYEPQK

SEQ ID NO: 88 - protein, 250 aa, Candida tropicalis
MKFSLALLTTVAAALVVAAPTQAPVEEAEVPTNETGLAIPDSAVCAIVPLDGELAPVFVELDDIPVLMIVNTTAVEEAYQAEEEAYEAEEGSSDVEKRDAAKFKFRLTRYGWFSPNKREEIDAEDIIDAEKRDAAKFKFRLTRYGWFSPNKRDIGDEEDIVDAEKRDAAKFKFRLTRYGWFSPNKRELAEEEETVDAEKRDAAKFKFRLTRYGWFSPNKREVAEENDIVEKRDAAKFKFRLTRYGWFSPN

SEQ ID NO: 89 - protein, 402 aa, Dactylellina haptotyla
MQLKHTITILSLLAPLLNALPVAEPEPTAAPEAKAGSGDVMLPRSWCIYNSCPKNKRAPEPVAEPVAIPEPTAAPEPVIPAHIEARGVEAVRRWCVYNSCPKTKREAAPAPEPTAEPEPVIPAHIEARGEEYVKRWCVYNSCPKTKRAAEPIPEPTAQPEPIIPDHVQAQGEEFVKRWCVYNSCPKTKREAQPEPTAAPEPVIPDHIQARGEEYIKRWCVYNSCPKTKREAQPEPTAAAEAGIPAHIQARGEEYVKRWCVYNSCPKTKREAMPEPTAAPEPVIPDHIQARGEEFVKRWCVYNSCPKTKREAAPAPAPTAAPEPVIPAHIQARGEEYVKRWCVYNSCPKTKREALPAPTAAPEPIPAPEAEKMEPRSWCIYNSCPKYKRAAQPVPEPTAMPVA

SEQ ID NO: 90 - protein, 505 aa, Fusarium graminearum
MKYSILTLAAVASTTLAVAVPAPQPDPVAEPMPWCTWKGQPCWKEKMARREAQPEPEAVAAPEPDPVAEPMPWCTWKGQPCWKEKMARRAAQPEPEAVAAPEPDPVAEPMPWCTWKGQPCWKEKMKMAKREAQPEPEAVAAPEPDPVAEPMPWCTWKGQPCWKEKMAKRAAEAEAEPEPIPAPQPDPVAEAEPWCTWKGQPCWKAKMAKRAAEAEAEAEPIPDPVAAPQPDPVAEPMPWCTWKGQPCWKEKMAKREAKPEPWCWWKGQPCWKAKRDAAPEPWCWWKGQPCWKAKRNAAPEPMPEPANEPRWCWWKGQPCWKSKSKRDASPEPWCWWKGQPCWKAKRDAGEALTVALHATRGVETRSVAETEHLPRDAAHQAKRSIVELANVIALSARGSPEEYFKHLYLEEFFPEIPHNATAKRDVKTLQEDKRWCWWKGQPCWKAKRAAEAVLHAVDGSDGAGAPGGPEEHFDTSHFNPQNFEAKRDLMAIKAAARSVVESLEG

SEQ ID NO: 91 - protein, 148 aa, Geotrichum candidum
MRFSLATVYAFTVIGTVLGVPIASSEPTATTLSTVAAASATFSPGGDSPFTGIKNFPDFASFPPFPPGFDTGLSKRSADASPGDWGWFWYVPRPGDPAMKKRDALAEANPEANPGDWGWFWYVPRPGDPAMKKRDALADANPDANPVE

SEQ ID NO: 92 - protein, 285 aa, Hypocrea jecorina
METKEKTVVPKSKSPLSIYFSLDRVSLHPSSLLISPSPSHLLSPSPHIAKLQTMKFLAAVTVFASAALAAPNPEPWCYRIGEPCWKLKRTAEAFNLAVRSHDLTTRAQGEAIPDEVALSAIEGLDQLKKLILVSTEDPSSLLPPNATEPESKRDVEVEEDKRWCYRIGEPCWKAKREAEAEAAAEEEKRWCYRIGEPCWKAKRTDEISEEKRWCWILGGKCWKTKRVAEAVLSATIEGDEKRSVEAEGNADEKRWCYRIGEPCWKAKRDLETIQDVARSVIESMQ

SEQ ID NO: 93 - protein, 187 aa, Kluyveromyces lactis
MKFSTILAASTALISVVMAAPVSTETDIDDLPISVPEEALIGFIDLTGDEVSLLPVNNGTHTGILFLNTTIAEAAFADKDDLKKREADASPWSWITLRPGQPIFKREANADANAEASPWSWITLRPGQPIFKREANADANADASPWSWITLRPGQPIFKREANPEAEADAKPSAWSWITLRPGQPIF

SEQ ID NO: 94 - protein, 398 aa, Komagataella pastoris
MKSLILNIISVTLAITSTAASAPVESIFANQPDSSLTDTNDGVGVGMSTIKEEDFGKHFVENQILDEAVIMSLKLRKGVNLFFLDDIGLATELIGNKIAQIEAIDLSERLAQSWTNIRKNRLFGKREAEAEAEAEAFRWRNNEKNQPFGKREAEAEAEAEAEAEAEAEAFRWRNNEKNQPFGKREAEAEAEAEAEAEAEAFRWRNNEKNQPFGKREAEAEAEAEAFRWRNNEKNQPFGKREADAEAEAEAEAFRWRNNEKNQPFGKREAEAEAEAEAFRWRNNEKNQPFGKREAEAEAEAEAEAEAFRWRNNEKNQPFGKREAEAEAEAEAFRWRNNEKNQPFGKREADAEAEAEAEAFRWRNNEKNQPFGKREASIDTGTDDGAYWSWRKNSVLERQ

SEQ ID NO: 95 - protein, 324 aa, Lodderomyces elongisporous
MKFSTAVLTAIAVTLVAAAPVDIDTNANAADNVIEATTSNEEAAIPETTEIALDNAEQITDEQIPSDCGLELGPETQIEGELPQEDGEEGYYVYIPDTENFANEEEAAQYYQKRSADPGWMWTRYGRFSPVKRDANAEAEAEANADPGWMWTRYGRFSPVKRDANAEAEAEDKAEANADPGWMWTRYGRFSPVKRDANAEAEAEANADPGWMWTRYGRFSPVKRDANAEAEANADPGWMWTRYGRFSPVKRDANAEADPGWMWTRYGRFSPVKRDANAEADANAEADPGWMWTRYGRFSPVKRDANAEADPGWMWTRYGRFSPV

SEQ ID NO: 96 - protein, 358 aa, Mycosphaerella graminicola
MKLAVSTVLMVAVTLTQALAVADAEPKRRRGNSFVGWCGAIGAPCAKVKRDAEAMPDPKKRRGNSFTGWCGAIGAPCARVKRSADAIAEAFAYPEADPKKRRGNSFVGWCGAIGAPCAKAKRDIIEVGESVEEAVHDVYAREAEAEADPKKRRGNSFVGWCGAIGAPCAKRDLFSEVETDVSAEDSEDEDAIYARDAAPEARRKKKAKKPKRRGHRGNSFVGWCGALGAPCAKVKRDADAVAFAEAKKQRGNSFTGWCGAIGAPCAKDKREEHEILKTDVCEADDGECKALRNAYEAFHEIKARDAELEAENLASIDDDDELTKREVEVCNEPDGECDLAKRALDTIEAKLDAAIKAL

SEQ ID NO: 97 - protein, 321 aa, Magnaporthe oryzea
MKTVSVITLILGAGAAANAAAIVNAETLEARSEDAATLEARQWCPRRGQPCWKVKRAVDAFASAMHSNEARDVATTTSPSDGHLTARDLSHLPGGAAYNAKRSVNALAALLASTQYDPEAFYNDLYLDRYFDPDTSVDAKAVDEKPDAEAKTEKRDEEGGHLEARQWCPRRGQPCWKRDVEHDKRHCNSAGEACDVAKRAVGALLSAVEDSGADLAKRQWCPRRGQPCWKRDNVFEPVALGRRDVSDAEADVLTKRQWCPRRGQPCWKRSEISGLEARCYGPAGECTKAQRDLNAIHLAARDVLASLDFGRHLSSRLLDHS

SEQ ID NO: 98 - protein, 299 aa, Neurospora crassa
MKFTLPLVIFAAVASATPVAQPNAEAEAQWCRIHGQSCWKVKRVADAFANAIQGMGGLPPRDESGHQPAQVAKRQVDELAGIIALTQEDVNAYYDSLSLQEKFAPSTEEEKKTEKVAKREAEAEAQWCRIHGQSCWKKREAEAQWCRIHGQSCWKRDALPEAEPQWCRIHGQSCWKKRDAAPEAAPEAEANPQWCRIHGQSCWKAKRAAEAVMTAIQSAEAESALLLRDTTFSPVDRVGKRDPQVCNMRLHPKKVCWKRDASPEAACNAPDGSCTKATRDLHAMYNVARAILTAHSDEN

SEQ ID NO: 99 - protein, 271 aa, Pseudogymnoascus destructans
MKYLATLCVAALVAGVNSAAIAAAEPFCWRLGQPCDKVKRAAEAFAEAFDEPIAEAEAFDEPIAEAEASAFCWRPGQICEKAKRAALALAHTVADANPEAEAFFDKLAIDEAFPEPEAVADAEIADKVKREAEAEAFCWRPGQPCGKVKRAADAIASALAEPAPEPFCQRPGQLCGKVKRDAEAVAEAFCWRPGQPCGKAKREANALAEAAAEALEFGGLEKEQNSKRIFRPPHYTTTAIFPTDPRLFHHFHEEQPYDCRKVDPNCVTVEA

SEQ ID NO: 100 - protein, 111 aa, Penicillium chrysogenum
MKFTSVVVAVIAAGTVQAAALAPSETLPKWCGHIGQGCKRTTDASLDVKRSADALAEAMAGGLPLVLQKWCGHIGQGCYKAKRAADAVDEVKRTSDALARAFAALEEEDDE

SEQ ID NO: 101 - protein, 144 aa, Saccharomyces cerevisiae
MRFPSIFTAVLFAASSALAAPVNTTTEDETAQIPAEAVIGYLDLEGDFDVAVLPFSNSTNNGLLFINTTIASIAAKEEGVSLDKREAEAWHWLQLKPGQPMYKREAEAEAWHWLQLKPGQPMYKREADAEAWHWLQLKPGQPMY

SEQ ID NO: 102 - protein, 205 aa, Saccharomyces castellii
MKLSALLSTVALASTSFAAPIDTTASNENLNSTDIPAEAVIGYLDLGSDSDVAMLPFQNSTSNGLLFVNTTIVQQAAQENDDSVGLAKREANAEAGWHWLRLDPGQPLYKREADADAEANWHWLRLDPGQPLYKREAEADAEANWHWLRLDPGQPLYKREADADAEANWHWLRLDPGQPLYKREADADAEANWHWLRLDPGQPLY

SEQ ID NO: 103 - protein, 242 aa, Sporothrix scheckii
MKTAAVFTILAVGASAAAVAEAEAYCQSVGQSCYQVKRAAEAFAEAIADLGAPEAGISRRSLSFGGVHNNAIRAIDGLASIVASTQYNPRSFYSDLSLESHFPVPVEEPVTKREAEADADADAQRYCPLPGQPCWKNKREAEAAADAEAQKYCPLKGQSCWKARRAAEAVINAIEGGSVQKREAEADAEAQKYCPLKGQSCWKRNVGTRCYAPGGACANASRDLHAIYNAARSVIESLPKAE

SEQ ID NO: 104 - protein, 213 aa, Schizosaccharomyces japonicus
MKFSAIFILSLFASAFAAPVPSSDAVEAAAPIIPELLSTEQVVLEGRVSDRVKQMLSHWWNFRNPDTANLKRSEPERRVSDRVKQMLSHWWNFRNPDTANLKRSEPERRVSDRVKQMLSHWWNFRNPDTANLKRSEPERRVSDRVKQMLSHWWNFRNPDTANLKKRALTDAQEEEAESEMDLLSYLLYSNDTSIAASGLNATEMVETILKDYE

SEQ ID NO: 105 - protein, 125 aa, Saccharomyces kluyveri
MKLFTTLSASLIFIHSLGSTRAAPVTGDESSVEIPEESLIGFLDLAGDDISVFPVSNETHYGLMLVNSTIVNLARSESANFKGKREADAEPWHWLSFSKGEPMYKREADAEPWHWLSFSKGEPMY

SEQ ID NO: 106 - protein, 182 aa, Phaeosphaeria nodorum
MRFNAVIAACILAVTVSGAALPTEDAAITDAATITTTEAEITEAEIIKAAPEEDDFFDDDEQFEKRDAASWKYNGWRYRPYGLPVGKRDADAEAGWRYRPYGLPVGKREAAPEADAEAKYNGWRYRPYGLPVGKREAEAKYNGWRYRPYGLPVGKREAEADASAEARYNGWRYRPYGLPVGR

SEQ ID NO: 107 - protein, 310 aa, Schizosaccharomyces octosporus
MKFFSLVALLFALASAAPIPATSKDSGVSPLDQLPSKTYEDFLRVYKNWQTFQNPDRPDLKKRDVPELPSKTYEDFLRVYKNWWSFQNPDRPDLKKRDVEELPAKTYEDFLRVYQNWETFQNPDRPDLKKRDVPELPSKTYEDFLRVYKNWWSFQNPDRPDLKKRDVEELPAKTYEDFERVYQNWETFQNPDRPDLKKRDVPELPSKTYEDFLRVYKNWWSFQNPDRPDLKKRDVPELPSKTYEDFLRVYKNWWSFQNPDRPDLKKRDVEEPVLKTEKDKEDYYHFLEFYVMNVPFNSTVAQTNISSHFD

SEQ ID NO: 108 - protein, 201 aa, Schizosaccharomyces pombe
MKITAVIALLFSLAAASPIPVADPGVVSVSKSYADFLRVYQSWNTFANPDRPNLKKREFEAAPAKTYADFLRAYQSWNTFVNPDRPNLKKREFEAAPEKSYADFLRAYHSWNTFVNPDRPNLKKREFEAAPAKTYADFLRAYQSWNTFVNPDRPNLKKRTEEDEENEEEDEEYYRFLQFYIMTVPENSTITDVNITAKFES

SEQ ID NO: 109 - protein, 143 aa, Scheffersomyces stipitis
MHLRSTAILSAVVFTSVALSAPTSGQNIDIDFPDESIAGAIPLSYDLVPIIGSYQGQNVILIVNSTIAAASEAAASEGKSKRDANAWHWTSYGVFEPGKRDANANAAPWHWTSYGVFEPGKRDANADAAPWHWTSYGVFEPGK

SEQ ID NO: 110 - protein, 134 aa, Torulaspora delbrueckii
MKFFNTILSTTLFTYVALAAPVESDPVNIPSEAILGYMDFTEDQDVGVVAYTNSTFSGLIFFNSSIIETKDLTKRDAEAGWMRLRLGQPLKKRDADADADAGWMRLSPGKPMKKREADADAEAGWMRLRIGQPL

SEQ ID NO: 111 - protein, 135 aa, Tuber melanosporum
MKVTILFLATLLSAALSEPIPWEVNGNRGVYRREPEAEAEAWHPRAGDPMAIWQKRNAEPYPEAEPEAIPWTPRPGRGAYRRHARPWTPRPGRGAYRRSAEAWHPRAGPPAYTLSKRDAAPEPVRFQPIGSFYKE

SEQ ID NO: 112 - protein, 206 aa, Vanderwaltozyma polyspora
MKLTNVLSAVALASTALAAPVAKDATNTTDASSVQIPAEAVIGYLDLEQSNDVAMLQFSNSTNNGILFVNSTILKAAYAEANANSNSNTKREAKADAWHWLELDNGQPIYKREANAEAKPWHWLELDNGQPIYKREAKAEAKADAWHWLELDNGQPIYKREAKAEAKADAWHWLELDNGQPIYKREAEAKAGAWHWLELDNGQPIY

SEQ ID NO: 113 - protein, 203 aa, Vanderwaltozyma polyspora
MKFSTVLSTVALAATAVSAAPISRASNETVESVESGLNVPAEAVLGYLDFGEKDDVAMLPFSNGTSNGLLFVNTTIYDAAFADSDDESASLAKRDAEAWHWLRLRYGEPIYKREDSEGVEKREAAAEPWHWLRLRYGEPIYKREDSESVEKREAAAEPWHWLRLRYGEPIYKREDSESVEKREANADADAWHWLRLRYGEPIY

SEQ ID NO: 114 - protein, 214 aa, Yarrowia lipolytica
MKFSTIALAAVACLVSAAPAAPVGTGSHGPQSIPEEAIVGGLQGTENEIFVFFNDDESGKQGIAIIDAKKAQEAGFMDPQPDSEVAAGNAKREASPEAWRWFWLPGYGEPNWKRDAMPADMDKEKREANPEAWRWFWLPGYGEPNWKRDAMPADMDKEKREANPEAWRWFWLPGYGEPNWKRDAMPADMDKEKREANPEAWRWFWLPGYGEPNW

SEQ ID NO: 115 - protein, 422 aa, Zygosaccharomyces bailii
MRFSITLCSTLCALTVAAAPIEEYKRAPVAEAEAAHLVRLSPGAAMFKREADADAEAEAAHLVRLSPGAAMFKREAEAEAEAEAAHLVRLSPGAAMFKREAEADADAEAEAAPLVRLSPGAAMFKREADADAEAEAAHLVRLSPGAAMFKREAEAEAEAEAAHLVRLSPGAAMFKREAEADADAEAEAAHLVRLSPGAAMFKREAEAEAAHLVRLSPGAAMFKREAEADAEAEAEAAPLVRLSPGAAMFKRKAEADAEAEAPPLVRLSPGAAMFKREAEADADAEAEAAHLVRLSPGAAMFKREAEADAEAEAAHLVRLSPGAAMFKREAEADADAEAEAAPLVRLSPGAAMFKREAEADAEAEAAHLVRLSPGAAMFKREAEADADAEAEAAHLVRLSPGAAMFKREAEADADAEAGADST

SEQ ID NO: 116 - protein, 187 aa, Zygosaccharomyces rouxii
MRLSIALGVTFGAVAGLTAPVEEVKRDADAHFIELDPGQPMFKREAEAHFIELDPGQPMFKREAEAEAHFIELDPGQPMFKREAEAEAHFIELDPGQPMFKREAEADAHFIELDPGQPMFKRDADAHFIELDPGQPMFKREAEAEAHFVELDPGQPMFKREAEADAHFIELDPGQPMFKRGEIESAA

SEQ ID NO: 117 - protein, 431 aa, Saccharomyces cerevisiae
MSDAAPSLSNLFYDPTYNPGQSTINYTSIYGNGSTITFDELQGLVNSTVTQAIMFGVRCGAAALTLIVMWMTSRSRKTPIFIINQVSLFLIILHSALYFKYLLSNYSSVTYALTGFPQFISRGDVHVYGATNIIQVLLVASIETSLVFQIKVIFTGDNFKRIGLMLTSISFTLGIATVTMYFVSAVKGMIVTYNDVSATQDKYFNASTILLASSINFMSFVLVVKLILAIRSRRFLGLKQFDSFHILLIMSCQSLLVPSIIFILAYSLKPNQGTDVLTTVATLLAVLSLPLSSMWATAANNASKTNTITSDFTTSTDRFYPGTLSSFQTDSINNDAKSSLRSRLYDLYPRRKETTSDKHSERTFVSETADDIEKNQFYQLPTPTSSKNTRIGPFADASYKEGEVEPVDMYTPDTAADEEARKFWTEDNNNL

SEQ ID NO: 118 - protein, 477 aa, Saccharomyces castellii
MSDAPPPLSELFYNSSYNPGLSIISYTSIYGNGTEVTFNELQSIVNKKITEAIMFGVRCGAAILTIIVMWMISKKKKTPIFIINQVSLFLILLHSAFNFRYLLSNYSSVTFALTGFPQFIHRNDVHVYAAASIFQVLLVASIEISLMFQIRVIFKGDNFKRIGTILTALSSSLGLATVAMYFVTAIKGIIATYKDVNDTQQKYFNVATILLASSINFMTLILVIKLILAIRSRRFLGLKQFDSFHILLIMSFQSLLAPSILFILAYSLDPNQGTDVLVTVATLLVVLSLPLSSMWATAANNASRPSSVGSDWTPSNSDYYSNGPSSVKTESVKSDEKVSLRSRIYNLYPKSKSEFEQSSEHTYVDKVDLENNFYELSTPITERSPSSIIKKGKQGISTRETVKKLDSLDDIYTPNTAADEEARKFWSEDVSNELDSLQKIETETSDELSPEMLQLMIGQEEEDDNLLATKKITVKKQ

SEQ ID NO: 119 - protein, 473 aa, Vanderwaltozyma polyspora
MSGIDDMGDKPDILGLFYDANYDPGQGILTFISMYGNTTITFDELQLEVNSLITSGIMFGVRCGAACLTLLIMWMISKNKKTPIFIINQCSLILIIMHSGLYFKNILSNLNSLSYILTGFTQNITKNNIHVFGAANIIQVLLVATIELSLVFQIRVMFKGDSFRKAGYGLLSIASGLGIATVVMYFYSAITNMIAVYNQTYNSTAKLFNVANILLSTSINFMTVVLIVKLFLAVRSRRYLGLKQFDSFHILLIMSCQTLIVPSILFILSYALSTKLYTDHLVVIATLLVVLSLPLSSMWASAANNSPKPSSFTTDYSNKNPSDTPSFYSQSISSSMKSKFPSKFIPFNFKSKDNSSDTRSENTYIGNYDMEKNGSPNHSYSSKDQSEVYTIGVSSMHTDIKSQKNISGQHLYTPSTEIDEEARDFWAGRAVNNSVPNDYQPSELPASILEELNSLDENNEGFLETKRITFRKQ

SEQ ID NO: 120 - protein, 384 aa, Vanderwaltozyma polyspora
MSSQSHPPLIDLFYDSSYDPGESLIYYTSIYGNNTYITFDELQTIVNKKVTQGILFGVRCGAAFLMLVAMWLISKNKRSRIFITNQCCLVFMIMHSGLYFRYLLSRYGSVTFILTGFQQLLTRNDIHIYGATDFIQVALVACIELSLIFQIKVIFAGTNYGKLANYFITLGSLLGLATFGMYMLTAINGTIKLYNNEYDPNQRKYFNISTILLASSINMLTLILILKLVAAIRTRRYLGLKQFDSFHILLIMSTQTLIIPSILFILSYSLREDMHTDQLIIIGNLIVVLSLPLSSMWASSLNNSSKPTSLNTDFSGPKSSEEGTAISLLSQNMEPSIVTKYTRRSPGLYPVSVGTPIEKEASYTLFEATDIDFESSSNDITRTS

SEQ ID NO: 121 - protein, 471 aa, Torulaspora delbrueckii
MSDSAQNLSDLAFNSSYNPLDSFITFTSIYGDNTAVKFSVLQDMVDVNTNEAIVYGTRCGASVLTQIIMWMISKNRRTPVFIINQVSLTLILIHSALYFKYLLSGFGSVVYGLTAFPQLIKPGDLRAFAAANIVMVLLVASIEASLIFQVKVIFTGDNMKRVGLILTIICTCMGLATVTMYFITAVKSIVSLYRDMSGSSTVLYNVSLIMLASSIHFMALILVVKLFLAVRSRRFLGLKQFDSFHILLIISCQTLLVPSLLFIIAYSFPSSKNIESLKAIAVLTVVLSLPLSSMWATAANNFTNSSSSGSDSAPTNGGFYGRGSSNLYPEKTDNRSPKGARNALYELRSKNNAEGQADIYTVTDIENDIFNDLSKPVEQNIFSDVQIIDSHSLHKACSKEDPVMTLYTPNTAIEGEERKLWTSDCSCSTNGSTPVKKKSTGEYANLPPHLLRYDENYDEEAGGRRKASLKW

SEQ ID NO: 122 - protein, 426 aa, Saccharomyces kluyveri
MSGKQDLSPLGLYSSYDPTKGLISYTSLYGSGTTVTFEELQIFVNKKITQGILFGTRIGAAGLAIIVLWMVSKNRKTPIFIINQISLFLILLHSSLFLRYLLGDYASVVFNFTLFSQSISRNDVHVYGATNMIQVLLVAAVEISLIFQVRVIFKGDSYKGVGRILTSISAVLGFTTVVMYFITAVKSMTSVYSDLTKTSDRYFFNIASILLSSSVNFMTLLLTVKLILAVRSRRFLGLKQFDSFHVLLIMSFQTLIFPSILFILAYALNPNQGTDTLTSIATLLVTLSLPLSSMWATSANNSSHPSSINTQFRQRNYDDVSFKTGITSFYSESSKPSSKYRHTNNLYDLYPVSRTSNSRCNGYPNDGSKLAPNPNCVGHNGSTMSVNDKNGAHATCVQNNVTLNTDSTLNYSNVDTQDTSKILMTT

SEQ ID NO: 123 - protein, 436 aa, Kluyveromyces lactis
MSEEIPSLNPLFYNETYNPLQSVLTYSSIYGDGTEITFQQLQNLVHENITQAIIFGTRIGAAGLALIIMWMVSKNRKTPIFIINQSSLVLTIVQSALYLSYLLSNFGGVPFALTLFPQMIGDRDKHLYGAVTLIQCLLVACIEVSLVFQVRVIFKADRYRKIGIILTGVSASFGAATVAMWMITAIKSIIVVYDSPLNKVDTYYYNIAVILLACSINFITLLLSVKLFLAFRARRHLGLKQFDSFHILLIMSTQTLIGPSVLYILAYALNNKGVKSLTSIATLLVVLSLPLTSIWAAAANDAPSASTFYRQFNPYSAQNRDDSSSYSYGKAFSDKYSFSNSPQTSDGCSSKELELSTQLEMDLESGESFMDRAKRSDFVSSPGSTDATVIKQLKASNIYTSETDADEEARAFWVNAIHENKDDGLMQSKTVFKELR

SEQ ID NO: 124 - protein, 443 aa, Zygosaccharomyces rouxii
MSEINNSTYNPMNAYVTFTSIYGDDTMVRFKDVELVVNKRVTEAIMFGVKVGAASLTLIIMWMISKKRTTPIFIINQSSLVFTIIHASLYFGYLLSGFGSIVYNMTSFPQLISSNDVRVYAATNIFEVLLVASIEISLVFQVKVMFANNNGRRWTWCLMVVSIGMALATVGLYFATAVELIRAAYSNDTVSRHVFYNVSLILLASSVNLMTLMLVVKLVLAIRSRRFLGLKQFDSFHILLIMSCQTLIAPSILFILGWTLDPHTGNEVLITVGQLLIVLSLPLSSMWATTANNTSSSSSSVSCNDSSFGNDNLCSKSSQFRRTFMNRFRPKSVNGDGNSENTFVTIDDLEKSVFQELSTPVSGESKIDHDHASSISCQKTCNHVHASTVNSDKGSWSSDGSCGSSPLRKTSTVNSEDLPPHILSAYDDDRGIVESKKIILKKL

SEQ ID NO: 125 - protein, 452 aa, Zygosaccharomyces bailii
MSGLANNTSYNPLESFIIFTSVYGGDTMVKFEDLQLVFTKRITEGILFGVKVGAASLTMIVMWMISRRRTSPIFIMNQLSLVFTILHASFYFKYLLDGFGSIVYTLTLFPQLITSSDLHVFATANVVEVLLVSSIEASLVFQVNVMFAGSNHRKFAWLLVGFSLGLALATVALYFVTAVKMIASAYASQPPTNPIYFNVSLFLLAASVFLMTLMLTVKLILAIRSRRFLGLKQFDSFHILLIMSCQTLIAPSVLYILGFILDHRKGNDYLITVAQLLVVLSLPLSSMWATTANDASSGTSMSSKESVYGSDSLYSKSKCSQFTRTFMNRFSTKPTKNDEISDSAFVAVDSLEKNAPQGISEHVCEFPQSDLSDQATSISSRKKEAVVYASTVDEDKGSFSSDINGYTVTNMPLASAASANCENSPCHVPRPYEENEGVVETRKIILKKNVKW

SEQ ID NO: 126 - protein, 417 aa, Candida glabrata
MEMGYDPRMYNPRNEYLNFTSVYDVNDTIRFSTLDAIVKGLLRIAIVHGVRLGAIFMTLIIMFISSNTWKKPIFIINMVSLMLVMIHSALSFHYLLSNYSSISYILTGFPQLITSNNKRIQDAASIVQVLLVAAIEASLVFQIHVMFTIENIKLIREIVLSISIAMGLATVATYLAAAIKLIRGLHDEVMPQTHLIFNLSIILLASSINFMTFILVIKLFFAIRSRRYLGLRQFDAFHILLIMFCQSLLIPSVLYIIVYAVDSRSNQDYLIPIANLFVVLSLPLSSIWANTSNNSSRSPKYWKNSQTNKSNGSFVSSISVNSDSQNPLYKKIVRFTSKGDTTRSIVSDSTLAEVGKYSMQDVSNSNFECRDLDFEKVKHTCENFGRISETYSELSTLDTTALNETRLFWKQQSQCDK

SEQ ID NO: 127 - protein, 458 aa, Ashbya gossypii
MGEEVSSFVEQYYDPNYDPSQSMLTYMSKFSNESTIKFEDLQEYINENVMLGVFTGAKIAAAALALIILWMVTKRKRTPIYIVNQISLLLTVIHGILVLSGLLGGFSSSIFTLTLFPQCVNRSDIRLFVATNISMVSLIASIQVSLVLQVHVIFRAGTHRRLGIFLTAVSAIIGFTTVCFYLVSAVLSVMAVYQDIDNIGDTFFLSIAYICMAISVNFIFLLLSVKLLLAIRLRRFLGLKQFDGLHILFIMSTQTIICPSILFILAFACEKNITDSLVYIAVLLVSLSLPLSSVWATAANNATVPPFLNAHSLTSRYKAESWYTDSKNDAGSFSSSENCGSGYRHGRYSNNGGSSPHQCTGGDNTVIDIEKCQYRVNPTPHTSGQFAFNQDSLETEFSEDTVVQIRTPNTEVEEEAKIFWARASITHENSSSGVECGAHDMQTNVFKTPTSQTGSDCN

SEQ ID NO: 128 - protein, 321 aa, Scheffersomyces stipitis
MDTSINTLNPANIIVNYTLPNDPRVISVPFGAFDEYVNQSMQKAIIHGVSIGSCTIMLLIILIFNVKRKKSPAFYLNSVTLTAMIIRSALNLAYLLGPLAGLSFTFSGLVTPETNFSVSEATNAFQVIVVALIEASMTFQVFVVFQSPEVKKLGIALTSISAFTGAAAVGFTINSTIQQSRIYHSVVNGTPTPTVATWSWVRDVPTILFSTSVNIMSFILILKLGFAIKTRRYLGLRQFGSLHILLMMATQTLLAPSILILVHYGYGTSSNSQLILISYLLVVLSLPVSSIWAATANNSPQLPSSATLSFMNKTTSHFSES

SEQ ID NO: 129 - protein, 354 aa, Komagataella pastoris
MEEYSDSFDPSQQLLNFTSLYGETDATFAELDDYHFYVVKYAIVYGARIGVGMFCTLMLFVVSKSWKTPIFVLNQSSLILLIIHSGFYIHYLTNQFSSLTYMFTRIPNETHAGVDLRINVVTNTLYALLILSIEISLIYQVFVIFKGVYENSLRWIVTIFTALFAAAVVAINFYVTTLQSVSMYNSNVDFPRWASNVPLILFASSVNWACLLLSLKLFFAIKVRRSLGLRQFDTFHILAIMFSQTLIIPSILIVLGYTGTRDRDSLASLGFLLIVVSLPFSSMWAATANNSNIPTSTGSFAWKNRYSPSTYSDDTTAVSKSFTIMTAKDECFTTDTEGSPRFIKGDRTSEDLHF

SEQ ID NO: 130 - protein, 395 aa, Candida guilliermondii
MKSCSIGFGIPFINEPNFETVSILTMDVSFIDADVNPDNILLNFTIPGYQNGFSVPMVVINELQKSQMKYAIVYGCGVGASLILLFVVWILCSRKTPLFIMNNIPLVLYVISSSLNLAYITGPLSSVSVFLTGILTSHDAINVVYASNALQMLLIFSIQSTMAYHVYVMFKSPQIKYLRYMLVGFLGCLQIVTTCLYINYNVLYSRRMHKLYETGQTYQDGTVMTFVPFILFQCSVNFSSIFLVLKLIMAIRTRRYLGLRQFGGFHILMIVSLQTMLVPSILVLVNYAAHKAVPSNLLSSVSMMIIVLSLPASSMWAAAANASSAPSSAASSLFRYTTSDSDRTLETKSDHFIMKHESHNSSPNSSPLTLVQKRISDATLELPKELEDLIDSTSI

SEQ ID NO: 131 - protein, 403 aa, Candida parapsilosis
MNKIVSKLSSSDVIVTVTIPNEEDGTYEVPFYAIDNYHYSRMENAVVLGATIGACSMLLIMLIGILFKNFQRLRKSLLFNINFAILLMLILRSACYINYLMNNLSSISFFFTGIFDDESFMSSDAANAFKVILVALIEVSLTYQIYVMFKTPMLKSWGIFASVLAGVLGLATLATQIYTTVMSHVNFVNGTTGSPSQVTSAWMDMPTILFSVSINVLSMFLVCKLGLAIRTRRYLGLKQFDAFHILFIMSTQTMIIPSIILFVHYFDQNDSQTTLVNISLLLVVISLPLSSLWAQTANNVRRIDTSPSMSFISREASNRSGNETLHSGATISKYNTSNTVNTTPGTSKDDSLFILDRSIPEQRIVDTGLPKDLEKFINNDFYEDDGGMIAREVTMLKTAHNNQ

SEQ ID NO: 132 - protein, 376 aa, Candida auris
MEFTGDIVLKYTLGGEEYLSTFEQLDSSVNRSLELGVVHGIAIACGVLLMVLAWVIIIKKKNPIFVLNQLTLLLMVIKSSLYLAFLFGPLSSLTYKFTRVLPHDKWHAFHVYIATNVIHTLLIATVEMTLVFQIYIIFKSPEVRHLGYILTGAASALALTIVALYIHSTVISAVQLKEQLLMHEIKITNSWVNNVPIILFSASLNVVCIILIAKLALAIKTRRYLGLKQFDGLHILMITSTQTFIVPSVLMIVNYKQSSSYLTLLANISVILVVCNLPLSSLWAASANNSSTPTSSANTVFSRWDSKFSDTETIAHELPLIPGKAEKLQLVSPITEKGDTHTMCESHGDQDLIDKMLDDIEGAVMTTEFNLNNRTV

SEQ ID NO: 133 - protein, 369 aa, Yarrowia lipolytica
MQLPPRPDFDIATLVASITVPETELVLGQMPLGALEQLYQNRLRLAILFGVRVGAAVLTLIAMHLISKKNRTKILFLANQMSLIMLIIHAALYFRFLLGPFASMLMMVAYIVDPRSNVSNDISVSVATNVFMMLMIMSVQLSLAVQTRSVFHAWLKSRIYVTVGLILLSLVVFVFWTTHTIVSCIVLTHPTRDLPSMGWTRLASDVSFACSISFASLVLLAKLVTAIRVRKTLGKKPLGYTKVLVIMSTQSLVVPSILIIVNYALPEKNSWILSGVAYLMVVLSLPLSSIWATAVHDDEMQSNYLLSALKDGHVQPSESKLKTVFLNRLRPFSTTTNRDDESSVDSPAMPSPESDVTFLNTGFECDEKM

SEQ ID NO: 134 - protein, 360 aa, Candida lusitaniae
MNPADINIEYTLGDTAFSSTFADFEAWKTRNTQFAIVNGVALACGIILMVVSWIIIVNKRAPIFAMNQTMLVIMVIKSAMYLKHIMGPLNSLTFRFTGLMEESWAPYNVYVTINVLHVLLVAAVESSLVFQIHVVFKSSRARVAGRAIVSAMSTLALLIVSLYLYSTVRHAQTLRAELSHGDTTTVEPWVDNVPLILFSASLNVLCLLLALKLVFAVRTRRHLGLRQFDSFHILIIMATQTFVIPSSLVIANYRYASSPLLSSISIIVAVCNLPLCSLWACSNNNSSYPTSSQNTILSRYETETSQATDASSTTCAGIAEKGFDKSPDSPTFGDQDSVSISHILDSLEKDVEGVTTHRLT

SEQ ID NO: 135 - protein, 470 aa, Candida albicans
MNINSTFIPDKPGDIIISYSIPGLDQPIQIPFHSLDSFQTDQAKIALVMGITIGSCSMTLIFLISIMYKTNKLTNLKLKLKLKYILQWINQKIFTKKRNDNKQQQQQQQQQIESSSYNNTTTTTSGSYKLFLFYLNSLILLIGIIRSGCYLNYNLGPLNSLSFVFTGWYDGSSFISSDVTNGFKCILYALVEISLGFQVYVMFKTSNLKIWGIMASLLSIGLGLIVVAFQINLTILSHIRFSRAISTNRSEEESSSSLSSDSVGYVINSIWMDLPTILFSISINIMTILLIGKLIIAIRTRRYLGLKQFDSFHILLIGFSQTLIIPSIILVVHYFYLSQNKDSLLQQISLLLIILMLPLSSLWAQTANNTHNINSSPSLSFISRHHSSDSSRSGGSNTIVSNGGSNGGGGGGGNFPVSGIDAQLPPDIEKILHEDNNYKLLNSNNESVNDGDIIINDEGMITKQITIKRV

SEQ ID NO: 136 - protein, 412 aa, Candida tropicalis
MDINNTIQSSGDIIITYTIPGIEEPFELPFEVLNHFQSEQSKNCLVMGVMIGSCSVLLIFLVGILFKTNKFSTIGKSKNLSKNFLFYLNCLITFIGIIRAACFSNYLLGPLNSASFAFTGWYNGESYASSEAANGFRVILFALIETSMVFQVFVMFRGAGMKKLAYSVTILCTALALVVVGFQINSAVLSHRRFVNTVNEIGDTGLSSIWLDLPTILFSVSVNLMSVLLIGKLIMAIKTRRYLGLKQFDSFHVLLICSTQTLLVPSLILFVHYFLFFRNANVMLINISILLIVLMLPFSSLWAQTANTTQYINSSPSFSFISREPSANSTLHSSSGHYSEKSYGINKLNTQGSSPATLKDDHNSVILEATNPMSGFDAQLPPDIARFLQDDIRIEPSSTQDFVSTEVTYKKV

SEQ ID NO: 137 - protein, 419 aa, Candida tenuis
MDSYLLNHPGDISLNFALPLSDEVYTITFNDLDSQSSFSIQYLVIHSCAITVCLTLLVLLNLFIRNKKTPVFVLNQVILFFAIVRSSLFIGFMKSPLSTITASFTGIISDDQKHFYKVSVAANAALIILVMLIQVSFTYQIYIIFRSPEVRKFGVFMTSALGVLMAVTFGFYVNSAVASTKQYQHIFYSTDPYIMDSWVTGLPPILYSASVIAMSLVLVLKLVAAVRTRRYLGLKQFSSYHILLIMFTQTLFVPTILTILAYAFYGYNDILIHISTTITVVLLPFTSIWASIANNSRSLMSAASLYFSGSNSSLSELSSPSPSDNDTLNENVFAFFPDKLQKMNSSEAVSAVDKVVVHDHFDTISQKSIPHDILEILQGNEGGQMKEHISVYSDDSFSKTTPPIVGGNLLITNTDIGMK

SEQ ID NO: 138 - protein, 435 aa, Lodderomyces elongisporous
MDEAINANLVSGDIIVSFNIPGLPEPVQVPFSEFDSFHKDQLIGVIILGVTIGACSLLLILLLGMLYKSREKYWKSLLFMLNVCILAATILRSGCFLDYYLSDLASISYTFTGVYNGTSFASSDAANVFKTIMFALIETSLTFQVYVMFQGTTWKNWGHAVTALSGLLSVASVAFQIYTTILSHNNFNATISGTGTLTSGVWMDLPTLLFAASINFMTILLLFKLGMAIRQRRYLGLKQFDGFHILFIMFTQTLFIPSILLVIHYFYQAMSGPFIINMALFLVVAFLPLSSLWAQTANTTKKIESSPSMSFITRRKSEDESPLAANDEDRLRKFTTTLDLSGNKNNTTNNNNNSNNINNNMSNINYPSTGLGEDDKSFIFEMEPSRERAAIEEIDLGARIDTGLPRDLEKFLVDGFDDSDDGEGMIAREVTMLKK

SEQ ID NO: 139 - protein, 435 aa, Geotrichum candidum
MAEDSIFPNNSTSPLTNPIVVETIKGTAYIPLHYLDDLQYEKMLLASLFSVRIATSFVVIIWYFVAVNKAKRSKFLYIVNQVSLLIVFIQSILSLIYVFSNFSKMSTILTGDYTGITKRDINVSCVASVFQFLFIACIELALFIQATVVFQKSVRWLKFSVSLIQGSVALTTTALYMAIIVQSIYATLNPYAGNLIKGRFGYLLASLGKIFFSISVTSCMCIFVGKLVFAIHQRRTLGIKQFDGLQILVIMSTQSMIIPTIIVLMSFLRRNAGSVYTMATLLVALSLPLSSLWAEAKTTRDSASYTAYRPSGSPNNRSLFAIFSDRLACGSGRNNRHDDDSRGNGSVNARKADVESTIEMSSCYTDSPTYSKFEAGLDARGIVFYNEHGLPVVSGEVGGSSSNGTKLGSGHKYEVNTTVVLSDVDSPSPTDVTRK

SEQ ID NO: 140 - protein, 347 aa, Baudoinia compniacensis
MASNGWQNNATFDPYAQTFVLLQPDGLTPFPALLGDVLALNTVSVTQGIIYGTQVGISGLLLLILLIMTKPDKRRSLVFILNSLSLLLIFARNVLSCVQLTTIFYNFYNWELHWYPESPALSRAMDLSAATEVLNIPIDVAIFSSLVVQVHIVCCTIHTLVRTSALLSSAAVGLAAVAVRFALAVVNIKYSIFGINTLTEPQFNLIVHLKRVSDILTVVAIAFFSSIFVAKLGVAIHTRRTLNLKNFGAIQIIFIMGCQTMLIPLIFVIVSFYASRGSQIGSMVPTVVATFLPLSGMWASAQTNNEKMGRADQRFHRAVPVGATDFSVTKARSAKASDTLDTLIGDD

SEQ ID NO: 141 - protein, 348 aa, Schizosaccharomyces octosporus
MREPWWKNYYTMNGTQVQNQSIPILSTQGYIQVPLSTIDKAERNRILTGMTVSAQLALGVLIMVMSILLSSPEKRKTPVFIVNSASIISMCIRAILMIVNLCSESYSLAVMYGFVFELVGQYVHVFDILVMIIGTIIIITAEVSMLLQVRIICAHDRKTQRIVTCISSGLSLIVVAFWFTDMCQEIKYLLWLTPYNNHQISGYYWVYFVGKILFAVSIMFHSAVFSYKLFHAIQIRKKIGQFPFGPMQCILIISCQCLFVPAIFTIIDSFIHTYDGFSSMTQCLLIVSLPLSSLWASSTALKLQSLKSTTSPGDTTQVSIRVDRTYDIKRIPTEELSSVDETEIKKWP

SEQ ID NO: 142 - protein, 367 aa, Tuber melanosporum
MEQIPVYERPGFNPHKQNITLFKHDGSTVTVGLHELDAMFTHSIRVAVVFASQIGACALLSVIVAMVTKREKRRALFFLHIISLLLVVVRSVLQILYFVGPWAETYNYVAYYYEDIPLSDKLISIWAGIIQLILNICILLSLILQVRVVYATSPKLNTIMTLVSCVIASISVGFFFTVIVQISEAILNGVGYDGWVYKVHRGVFAGAIAFFSFIFIFKLAFAIRRRKALGLQRFGPLQVIFIMGCQTMIVPAIFATLENGVGFEGMSSLTATLAVISLPLSSMWAAAQTDGPSPQSTPRDGYRRFSTRRSALNRSDPSGGRSVDMNTLDSTGNDSLALHVDKTFTVESSPSSQSQAGPHKERGFEFA

SEQ ID NO: 143 - protein, 369 aa, Aspergillus oryzae
MDSKFDPYSQNLTFHAADGTPFQVPVMTLNDFYQYCIQICINYGAQFGASVIIFIILLLLTRPDKRASSVFFLNGGALLLNMGRLLCHMIYFTTDFVKAYQYFSSDYSRAPTSAYANSILGVVLTTLLLVCIETSLVLQVQVVCANLRRRYRTVLLCVSILVALIPVGLRLGYMVENCKTIVQTDTPLSLVWLESATNIVITISICFFCSIFIIKLGFAIHQRRRLGVRDFGPMKVIFVMGCQTLTVPALLSILQYAVSVPELNSNIMTLVTISLPLSSIWAGVSLTRSSSTENSPSRGALWNRLTDSTGTRSNQTSSTDTAVAMTYPSNKSSTVCYADQSSVKRQYDPEQGHGISVEHDVSVHSCQRL

SEQ ID NO: 144 - protein, 348 aa, Schizosaccharomyces pombe
MRQPWWKDFTIPDASAIIHQNITIVSIVGEIEVPVSTIDAYERDRLLTGMTLSAQLALGVLTILMVCLLSSSEKRKHPVFVFNSASIVAMCLRAILNIVTICSNSYSILVNYGFILNMVHMYVHVFNILILLLAPVIIFTAEMSMMIQVRIICAHDRKTQRIMTVISACLTVLVLAFWITNMCQQIQYLLWLTPLSSKTIVGYSWPYFIAKILFAFSIIFHSGVFSYKLFRAILIRKKIGQFPFGPMQCILVISCQCLIVPATFTIIDSFIHTYDGFSSMTQCLLIISLPLSSLWASSTALKLQSMKTSSAQGETTEVSIRVDRTFDIKHTPSDDYSISDESETKKWT

SEQ ID NO: 145 - protein, 369 aa, Aspergillus fischeri
MNSTFDPWTQNITLTQSDGTTVISSLALADDYLHYMIRLGINYGAQLGACAVLLLVLLLLTRPEKRVSSVFVLNVAALLANIIRLGCQLSYFSTGFARMYALLAGDFSRVSRGAYAGQVMASVFFTIVFICVEASLVLQVQVVCSNLRRQYRILLLGASTLAALVPIGVRLTYSVLNCMVIMHAGTMDHLDWLESATNIVTTVSICFFCAVFVVKLGLAIKMRKRLGVKQFGPMRVIFIMGCQTMTIPAIFAICQYFSRIPEFSHNVLTLVIISLPLSSIWAGFALVQANSTARSTESRHHLWNILSSDGATRDKPSQCVSSPMTSPTTTCYSEQSTSKPQQDPENGFGISVAHDISIHSFRKDAHGDI

SEQ ID NO: 146 - protein, 397 aa, Pseudogymnoascus destructans
MSTANVHLPADFDPTRQNITIYTPDGTPVVATLPMINLFNRQNNEICVVYGCQLGASLIMFLVVLLTTRVSKRKSPIFVLNVLSLIISCLRSLLQILYYIGPWTEIYRYLSFDYSTVPASAYANSVAATLLTLFLLITIEASLVLQTNVVCKSMSSHIRWPVTALSMVVSLLAISFRFGLTIRNIEGILGATVKSDSLMFSGASLISETASIWFFCTIFVIKLGWTLYQRKKMGLKQWGPMQIITIMAGCTMLIPSLFTVLEFFPEETFYEAGTLAICLVAILLPLSSVWAAAAIDGDEPVRPHGSTPKFASFNMGSDYKSSSAHLPRSIRKASVPAEHLSRTSEEELGDDGTLNRGGAYGMDRMSGSISPRGVRIERTYEVHTAGRGGSIEREDIF

SEQ ID NO: 147 - protein, 346 aa, Schizosaccharomyces japonicus
MYSWDEFRSPKQAEVLNQTVTLETIVSTIQLPISEIDSMERNRLLTGMTVAVQVGLGSFILVLMCIFSSSEKRKKPVFIFNFAGNLVMTLRAIFEVIVLASNNYSIAVQYGFAFAAVRQYVHAFNIIILLLGPFILFIAEMSLMLQVRIICSQHRPTMITTTVISCIFTVVTLAFWITDMSQEIAYQLFLKNYNMKQIVGYSWLYFIAKITFAASIIFHSSVFSFKLMRAIYIRRKIGQFPFGPMQCIFIVSCQCLIVPAIFTLIDSFTHTYDGFSSMTQCLLIISLPLSSLWATHTAQKLQTMKDNTNPPSGTQLTIRVDRTFDMKFVSDSSDGSFTEKTEETLP

SEQ ID NO: 148 - protein, 356 aa, Paracoccidioides brasiliensis
MAPSFDPFNQNVVFHKADGTPFNVSIHELDDFVQYNTRVCINYSSQLGASVIAGLMLAMLTHSEKRRLPVFFLNTFALAMNFARLLCMTIYFTTGFNKSYAYFGQDYSQVPGSAYAASVLGVVFTTLLVISMEMSLLIQTRVVCTTLPDIQRYLLMAVSSAISLMAIGFRLGLMVENCIAIVQASNFAPFIWLQSASNITITISTCFFSAVFVTKLAYALVTRIRLGLTRFGAMQVMFIMSCQTMVIPAIFSILQYPLPKYEMNSNLFTLVAIFLPLSSLWASVATKSSFETSSSGRHQYLWPSEQSNNVTNSEIKYQVSFSQNHTTLRSGGSVATTLSPDRLDPVYSEVEAGTKA

SEQ ID NO: 149 - protein, 422 aa, Mycosphaerella graminicola
MVVTAPPSVDRTYFIPNSTFDPYQQDLTLVYPDGVHALVANVDDIVYFMGLAVKSTLIFAIQIGISFVLMLVIALLTKPERRVTLVFFLNMTALFTIFIRAILMCTTFVGTYYNFYNWIMGNYPNSGLADRVSIAAEVFAFLIILSLELSMMFQVRIVCINLSSFRRRIITFSSIVVAMIVCTVRFALMVLSCDWRIVNIGDATQEKNRIINRVASGYNICTIASIIFFNTIFVSKLAVAIKHRRSMGMKQFGPMQIIFVMGCQTLLIPAIFGIISYFALASTQVYSLMPMVVAIFLPLSSMWASFNTNKTNSVTNMRQPNVYRPNMIIGQDTTQNSGKNTNISGTSNSTATTSSFASDKRRLNLSFNTQGTLVNSISEEEVNNPQKLGPSATVAVMDRDSLELEMRQHGIAQGRSYSVRSD

SEQ ID NO: 150 - protein, 380 aa, Penicillium chrysogenum
MATSSPIQPFDPFTQNVTFRLQDGTEFPVSVKALDVFVMYNVRVCINYGCQFGASFVLLVILVLLTQSDKRRSAVFILNGLALFLNSSRLLFQVIHFSTAFEQVYPYVSGDYSSVPWSAYAISIVAVVLTTLVVVCIEASLVIQVHVVCSTLRRRYRHPLLAISILVALVPIGFRCAWMVANCKAIIKLTYTNDVWWIESATNICVTISICFFCVIFVTKLGFAIKQRRRLGVREFGPMKVIFVMGCQTMVVPAIFSITQYYVVVPEFSSNVVTLVVISLPLSSIWAGAVLENARRTGSQDRQRRRNLWRALVGGAESLLSPTKDSPTSLSAMTAAQTLCYSDHTMSKGSPTSRDTDAFYGISVEHDISINRVQRNNSIV

SEQ ID NO: 151 - protein, 430 aa, Aspergillus nidulans
MATHNQISDQCQWSYPEVFTTQAVEEPTAEPASYHLHSTLTIMASNFDPWNQTITFRLEDGTPFDISVDYLDGILQYSIRACVNYAAQLGASVILFVILVLLTRAEKRASCLFWLNSLALLLNFARLLCDVLFFTGNFVRIYTLISADESRVTASDLATSIVGAIMTALLLTTIEISLVLQVQVVCSNLRRIYRRALLCVSAVVATATIAIRYSLLAVNIRAILEFSDPTTYNWLESLATVALTISICYFCVIFVTKLGFAIRLRRKLGLSELGPMKVVFIMGCQTLVIPGKRTLSSLIPPVIVSITHYVSDVPELQTNVLTIVALSLPLSSIWAGTTIDKPVTHSNVRNLWQILSFSGYRPKQSTYIATTTTATTNAKQCTHCYSESRLLTEKESGRNNDTSSKSSSQYGIAVEHDISVRSARRESFDV

SEQ ID NO: 152 - protein, 370 aa, Phaeosphaeria nodorum
MASMVPPPDFDPYTQEFMVLGPDGQEIPISMQTVNEYRLYTARLGLAYGSQIGATLLLLLVLSLLTRREKRKSGIFIVNALCLVTNTIRCILLSCFVTSTLWHPYTQFSQDTSRVSKTDVNTSIAASIFTLIVTVLIMISLSVQVWVVCITTAPYQRYMIMGATTATAMVAVGYKAAFVITSIIQTLNGQDGGSYLDLVMQSYITQAVAISFYSCIFTYKLGHAIVQRRTLNMPQFGPMQIIFIMGSLFTGLQFVKNVDELGIITPTIVCIFLPLSAIWAGVVNEKVVGANGPDAHHRLLQGEFYRAASNSTYGSNSSGTVVDRSRQMSVCTCASSSPFVRKKSVAEWDDEAILVGREFGFSRGEVGERG

SEQ ID NO: 153 - protein, 296 aa, Hypocrea jecorina
MSSFDPYTQNITILVSPSSPPISIPIPVIDAFNDETASIITNYAAQLGAALAMLLVLLAATPTARLLRADGPSLLHALALLVCVVRTVLLIYFFLTPFSHFYQVWTGDFSQVPAWNYRASIAGTVLSTLLTVVTDAALVNQAWTMVSLFAPRTKRAVCVLSLLITLLAISFRVAYTVIQCEGIAELAAPRQYAWLIRATLIFNICSIAWFCALFNSKLVAHLVTNRGVLPSRRAMSPMEVLIMANGILMIVPVVFAILEWHHFINFEAGSLTPTSIAIILPLSSLAAQRIANTSSS

SEQ ID NO: 154 - protein, 398 aa, Botrytis cinerea
MASNSSNFDPLTQSITILMADGITTVSFTPLDIDFFYYYNVACCINYGAQAGACLLMFFVVVVLTKAVKRKTLLFVLNVLSLIFGFLRAMLYAIYFLQGFNDFYAAFTFDFSRVPRSSYASSVAGSVIPLCMTITVNMSLYLQAYTVCKNLDDIKRIILTTLSAIVALLAIGFRFAATVVNSVAILATSASSVPMQWLVKGTLVTETISIWFFSLIFTGKLVWTLYNRRRNGWRQWSAVRILAAMGGCTMVIPSIFAILEYVTPVSFPEAGSIALTSVALLLPISSLWAGMVTDEETSAIDVSNLTGSRTMLGSQSGNFSRKTHASDITAQSSHLDFSSRKGSNATMMRKGSNAMDQVTTIDCVVEDNQANRGLRDSTEMDLEAMGVRVNKSYGVQKA

SEQ ID NO: 155 - protein, 412 aa, Beauvaria bassiana
MDGSSAPSSPTPDPTFDRFAGNVTFFLADHITTTSVPMPVLNAYYDESLCTTMNYGAQLGACLVMLVVVVALTPAAKLARRPASALHLVGLLLCAVRSGLLFAYFVSPISHFYQVWAGDFSAVSRRYWDASLAANTLAFPLVVVVEAALINQAWTMVAFWPRAAKAAACACSAVIVLLTIGTRLAYTIVQNHAIVTAVPPEHFLWAIQWSAVMGAVSIFWFCAVFNVKLVCHLVANRGILPSISVVNPMEVLVMTNGTLMIIPSIFAGLEWAKFTNFESGSLTLTSVIIILPLGTLAAQRISGQGSQGYQAGHLFHEQQQQQARTRSGAFGSASQQSHPTNKVPSSITLSTSGTPITPQISAGSRPELPLVDRSERLDPIDLELGRIDAFRGSSDFSPSTARPKRMQRDNFA

SEQ ID NO: 156 - protein, 566 aa, Neurospora crassa
MASSSSPPADIFSGITQSLNSTHATLTLPIPPADRDHLENQVLFLFDNHGQLLNVTTTYIDAFNNMLVSTTINYATQIGATFIMLAIMLLMTPRRRFKRLPTIISLLALCINLIRVVLLALFFPSHWTDFYVLYSGDWQFVPPGDMQISVAATVLSIPVTALLLSALMVQAWSMMQLWTPLWRALVVLVSGLLSLVTVAMSFANCIFQAKNILYADPLPSYWVRKLYLALTTGSISWFTFLFMIRLVMHMWTNRSILPSMKGLKAMDVLIITNSILMLIPVLFAGLEFLDSASGFESGSLTQTSVVIVLPLGTLVAQRIATRGYMPDSLEASSGPNGSLPLSNLSFAGGGGGGSGGHKDKENGGGIIPPTTNNTAATNFSSSIACSGISCLPKVKRMTASSASSSQRPLLTMTNSTIASNDSSGFPSPGIHNTTTTTTQYQYSMGMNMPNFPPVPFPGYQSRTTGVTSHIVSDGRHHQGMNRHPSVDHFDRELARIDDEDDDGYPFASSEKAVMHGDDDDDVERGRRRALPPSLGGVRVERTIETRSEERMPSPDPLGVTKPRSFE

SEQ ID NO: 157 - protein, 468 aa, Sporothrix scheckii
MKPAAGPASSPFDPFNQTFYLTGPDNTTVPVSVPQVDYIWHYIIGTSINYGSQIGACLLMLLVMLTLTSKSRFSRAATLINVASLLIGVIRCVLLAVYFTSSLTELYALFVGDYSQVRRSDLCVSAVATFFSLPQLVLIEAALFLQAYSMIKMWPSLWRAVVLAMSVVVAVCAIGFKFASVVMRMRSTLTLDDSLDFWLVEVDLAFTATTIFWFCFIYIIRLVIHMWEYRSILPPMGSVSAMEVLVMTNGALMLVPVIFAAIEINGLSSFESGSLVHTSVIVLLPLGSLIAQAMTRPDGYVQRTNTSGASGASGAHPGRNGSGHGGHGGAYSRAMTNTLNTLDTLDTVDSKTSIMHHHHHHHRNHSNGMSKTKANSGTWSHASDANSTNAMISGGIATQVRIQANQSTLGNTGMSGGSGAPNSHTRNNSLAAMEPVEKQLHDIDATPLSASDCRVWVDREVEVRRDMV

SEQ ID NO: 158 - protein, 415 aa, Magnaporthe oryzea
MDQTLSATGTATSPPGPALTVDPRFQTITMLTPALMGQGFEEVQTTPAEINDVYFLAFNTAIGYSTQIGACFIMLLVLLTMTAKARFARIPTIINTAALVVSIIRCTLLVIFFTSTMMEFYTIFSDDFSFVHPNDIRRSVAATVFAPLQLALVEAALMVQAWAMVELWPRAWKVSGIAFSLILATVTVAFKCASAAVTVKSALEPLDPRPYLWIRQTDLAFTTAMVTWFCFLFNVRLIMHMWQNRSILPTVKGLSPMEVLVMANGLLMVFPVLFAGLYYGNFGQFESASLTITSVVLVLPLGTLVAQRLAVNNTVAGSSANTDMDDKLAFLGNATTVTSSAAGFAGSSASATRSRLASPRQNSQLSTSVSAGKPRADPIDLELQRIDDEDDDFSRSGSAGGVRVERSIERREERL

SEQ ID NO: 159 - protein, 527 aa, Dactylellina haptotyla
MDHNTQHFNRPEYIEIPVPPSKGFNPHTNPAFFIYPDGSNMTFWFGQIDDFRRDQLFTNTIFSIQIGAALVILCVMFCVTHADKRKTIVYLLNVSNLFVVIIRGVFFVHYFMGGLARTYTTFTWDTSDVQQSEKATSIVSSICSLILMIGTQISLLLQVRICYALNPRSKTAILVTCGSISGIATTAYLLLGAYTIQLREKPPDMKFMKWAKPVVNALVALSIVSFSGIFSWRMFQSVRNRRRMGFTGIGSLESLLASGFQCLVFPGLVTTALTVAGSTWYIAVNLTTPSDLTAIYNCSAFFAYAFSIPLLKERAQVEKTISVVIAIAGVLVVAYGDGADDGSTSNGEKARLGGNVLIGIGSVLYGLYEVLYKKLLCPPSGASPGRSVVFSNTVCACIGAFTLLFLWIPLPLLHWSGWEIFELPTGKTAKLLGISIAANATFSGSFLILISLTGPVLSSVAALLTIFLVAITDRILFGRELTSAAILGGLLIIAAFALLSWATWKEMIEENEKDTIDSISDVGDHDD

SEQ ID NO: 160 - protein, 386 aa, Fusarium graminearum
MSKEAFDPFTQNVTFFAPDGKTEINIPVAAIDQVRRMMVNTTINYATQLGACLIMLVVILVMVPKEKFRRPFMILQIASLVICCCRMLLLSIFHSSQFLDFYVFWGDDHSRIPRSAYAPSVAGNTMSLCLVISVETMLMSQAWTMVRLWPNVWKYIIAGISLVVSIVAISVRLAYTIIQNNAVLKLEPAFHMFWLIKWTVIMNVASISWWCAIFNIKLVWHLISNRGILPSYKTFTPMEVLIMTNGILMIIPVIFASLEWAHFVDFESASLTLTSVAVILPLGTLAAQRIASSAPNSANSTGASSGIRYGVSGPSSFTGFKAPSFSTGTTDRPHVSIYARCEAGTSSREHINPQDVELAKLDPETDHHVRVDRAFLQREERIRAPL

SEQ ID NO: 161 - protein, 457 aa, Capronia coronata
MAARIIPALTLTAPTSYPTAGVGGYYYDTAFGVPTYSSAAFNQTTWRLLDNWDHINVNYASSEGLAAGLGWATLIYLLALTPSHKRTTPFHCFLLVGLIFLLGHLMVNIIAALTPGLNTTSAYTYVTLDTSSSVWPRKYIAVYAVNAVASWFAFIFATICLWLQAKGLMTGIRVRFIIVYKIILMYLIVAAVIALAICMAFNIQQILYIGKPVELADGTALLRLRNAYLITYAISIGSFSLVSICSIMDIIWRRPSRVIKGHNIFASALNLVGLLCAQSFVVPCEYKRALGQVPDCTTFADHIFHTVIFCILQVIPNSSGVMLPEIMLLPSVYVILPLGSLFMTVNSPESDVNKTSFPPKSSPGPFDRSPTLTSGTLPGSRPESYVLDMASDKNSGNRKSVCSQFDRELNLIDSLDTLSGREGDSMLHAQSNNNNQTREQDKQPRADTTHVGSENMV

SEQ ID NO: 162 - DNA, 664 bp, Artificial Sequence
  Description of Artificial Sequence Synthetic polynucleotide
agtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatactagtaaa

SEQ ID NO: 163 - DNA, 302 bp, Artificial Sequence
  Description of Artificial Sequence Synthetic polynucleotide
ctcgagacggctttgaaaaagtaatttcgtgaccttcggtataaggttactactagattcaggtgctcatcagatgcaccacattctctataaaaaaaaatggtatctttcttatttgataatatttaaactcctttacataataaacatctcgtaagtagtggtagaaaccacctttgcttttacgagttcaagcttttttcttgccatgatctagaactctcaggcaatatatacagttaatctttttttactgggttgtagttctaatgtattgtttcgaaaaatagcaaccaggcaca

SEQ ID NO: 164 - DNA, 100 bp, Artificial Sequence
  Description of Artificial Sequence Synthetic polynucleotide
gtatcctgctttgcaatgaaacaatagtatccgctaagaatttaagcaggccaacgtccatactgcttaggacctgtgcctggcaagtcgcagattgaag

SEQ ID NO: 165 - DNA, 100 bp, Artificial Sequence
  Description of Artificial Sequence Synthetic polynucleotide
ctcgagacggctttgaaaaagtaatttcgtgaccttcggtataaggttactactagattcaggtgctcatcagatgcaccacattctctataaaaaaaaa

SEQ ID NO: 166 - DNA, 100 bp, Artificial Sequence
  Description of Artificial Sequence Synthetic polynucleotide
ctatattattgtaccacattgccagatttatgaactctgggtatgggtgctaattttcgttagaagcgctggtacaattttctctgtcattgtgacacta

SEQ ID NO: 167 - DNA, 100 bp, Artificial Sequence
  Description of Artificial Sequence Synthetic polynucleotide
cacaagagtgtcgcattatatttactggactaggagtattttatttttacaggactaggattgaaatactgctttttagtgaattgtggctcaaataatg

SEQ ID NO: 168 - DNA, 1107 bp, Artificial Sequence
  Description of Artificial Sequence Synthetic polynucleotide
atgaactccaccttcgacccatggacccaaaacattactttgactcaatccgacggtaccactgtcatctcctctttggctttggccgatgactacttgcactacatgattagattgggtatcaactacggtgcccaattgggtgcttgtgctgttttgttgttggttttgttattgttgactagaccagaaaagagagtttcttctgtcttcgttttgaacgtcgctgctttgttggctaacatcatcagattgggttgtcaattgtcctacttctctaccggtttcgctagaatgtacgccttgttggccggtgacttctccagagtctctcgtggtgcttacgccggtcaagttatggcctccgtcttcttcaccattgtcttcatttgtgttgaagcttctttggttttgcaagttcaagtcgtctgttctaacttgagaagacaatacagaatcttgttattgggtgcttccactttggctgccttggttccaattggtgttcgtttgacttactccgttttaaactgtatggttattatgcacgctggtactatggaccacttggattggttggaatctgctaccaacatcgttactaccgtttctatttgtttcttctgtgctgttttcgttgtcaaattaggtttggctatcaagatgagaaagcgtttgggtgtcaaacaattcggtccaatgagagttatcttcatcatgggttgtcaaaccatgaccatcccagctattttcgctatttgtcaatacttctctagaattccagaattttctcataacgttttgactttggttatcatctctttgccattgtcttctatctgggccggttttgctttggtccaagccaactctaccgccagatctaccgaatctagacatcatttgtggaacattttgtcttccgatggtgctaccagagacaagccatcccaatgtgtttcttctccaatgacctctccaaccactacctgttactccgaacaatccacctctaagccacaacaagacccagaaaacggttttggtatttctgttgcccacgatatttccatccactctttcagaaaggacgcccacggtgatatt

SEQ ID NO: 169 - DNA, 1374 bp, Artificial Sequence
  Description of Artificial Sequence Synthetic polynucleotide
atgggtgaagaggtatctagctttgtggaacagtattatgatccaaactatgatcccagtcaatccatgctaacctacatgtcaaagttcagtaacgagtcgacaataaagtttgaggacttacaagagtatattaatgaaaacgtcatgttgggggtatttactggcgcaaagatagcggcagcagctctggcgttgataatcctatggatggtgactaaaaggaaaaggacacccatttacatcgttaaccagatatcactcctgcttacagtcatccatggcattctggtgttgtctggcttgctcggggggttttcttcttctatattcacactgacactattccctcaatgcgtgaatcggagtgatattcgcctgtttgtcgctaccaatatctccatggtttcgcttatagcctctatacaggtttcattggttctccaagttcacgtaatctttcgagcaggcactcacagacggttaggcatcttcttaactgcggtttccgctataatagggttcacaaccgtgtgcttttacctggtttctgctgtcctttcagtgatggctgtataccaggatatcgataacatcggcgatacattctttctgagcattgcgtacatttgtatggccatatctgtcaatttcatttttttgttactatccgttaagctgcttcttgcaatcagattaagacgcttcctaggtctaaaacaatttgatggcttacacatactcttcattatgtctactcagacaattatatgtccgagtattctgttcatactggctttcgcttgcgagaaaaatataacagattctttggtgtatattgcggtcttactcgtctcactgtcgctaccactgtcatctgtgtgggcaacagcagccaacaacgcaacagtcccaccttttttgaacgcccactctcttacttctaggtacaaagctgaatcctggtacacagattcaaagaatgatgcaggtagttttagctcctcagaaaattgtggatcgggatatcgacatggacgctattctaacaatgggggtagtagtccacatcaatgtacggggggggataataccgtcattgatatcgaaaaatgtcaatatagagtgaaccctacgccacatactagtgggcaattcgctttcaatcaggattcattggaaactgaattctcggaagataccgtcgtgcaaattcgtacgcccaatactgaggttgaagaggaggccaaaatattctgggcaagagccagtatcactcacgaaaatagttcttctggcgttgagtgcggtgcgcatgacatgcaaaccaacgtcttcaagactcctacaagtcaaaccggaagtgattgcaac

SEQ ID NO: 170 - DNA, 1290 bp, Artificial Sequence
  Description of Artificial Sequence Synthetic polynucleotide
atggctacccacaaccaaatctctgatcaatgtcaatggtcttacccagaagtcttcaccactcaagctgtcgaagaaccaaccgccgaaccagcttcttaccacttgcactctaccttgactattatggcttctaacttcgacccatggaaccaaaccattaccttcagattggaagacggtactccattcgacatttctgtcgactacttggacggtatcttgcaatactctatcagagcttgtgtcaactacgctgctcaattgggtgcttctgtcattttgtttgttatcttggtcttgttgactagagccgaaaaaagagcttcttgtttgttctggttaaactccttagctttgttgttgaacttcgccagattgttgtgtgacgtcttgttcttcaccggtaacttcgtcagaatttacactttgatctccgctgacgaatctagagttactgcttccgacttggctacttccatcgtcggtgctatcatgaccgctttgttgttgaccactattgaaatttctttggttttgcaagtccaagtcgtttgttctaacttgagaagaatctacagaagagccttgttgtgtgtttccgccgtcgttgccactgctaccattgctattagatactccttgttggctgtcaacattagagctattttggaattctccgacccaactacttacaactggttggaatctttagctaccgtcgccttgaccatctccatctgttacttctgtgtcatcttcgtcaccaagttaggtttcgctattagattgagaagaaagttgggtttatctgaattgggtccaatgaaggtcgtcttcatcatgggttgtcaaaccttggtcatcccaggtaaaagaaccttgtcttctttgattccaccagtcattgtttctattactcactacgtctccgacgtcccagaattgcaaactaacgttttgactatcgtcgccttgtccttgccattgtcctctatttgggctggtaccaccattgacaagccagtcactcactctaacgttagaaacttgtggcaaatcttgtccttctctggttacagaccaaagcaatctacctacattgctaccactactaccgctactaccaacgctaagcaatgtacccactgttactctgaatctagattgttgactgaaaaggaatctggtcgtaacaacgacacttcttctaagtcttcctcccaatacggtatcgctgtcgaacacgatatttccgttagatctgctcgtcgtgaatcttttgacgtc

SEQ ID NO: 171 - DNA, 1107 bp, Artificial Sequence
  Description of Artificial Sequence Synthetic polynucleotide
atggactctaagttcgacccatactctcaaaacttgactttccacgctgctgacggtaccccatttcaagttccagtcatgaccttgaacgacttttaccaatactgtattcaaatttgtatcaactacggtgctcaattcggtgcttccgtcatcattttcattatcttgttgttattgactagaccagacaaaagagcttcttctgttttcttcttaaacggtggtgccttgttgttgaacatgggtagattgttgtgtcacatgatttacttcactactgacttcgtcaaggcttaccaatacttctcttctgattactctagagccccaacctctgcctacgctaactccattttgggtgtcgtcttgaccaccttgttgttggtttgtatcgaaacctccttggttttacaagtccaagtcgtctgtgctaacttgagacgtagatacagaaccgtcttattgtgtgtttctatcttggtcgccttgatcccagtcggtttgagattgggttacatggttgaaaactgtaagactattgttcaaactgataccccattgtctttggtttggttggaatctgctactaacatcgtcattaccatctccatctgtttcttctgttctatcttcatcatcaagttgggtttcgccattcaccaaagaagaagattgggtgtcagagatttcggtccaatgaaggtcattttcgtcatgggttgtcaaactttgactgttccagctttgttgtctattttgcaatacgctgtctctgtcccagaattgaactctaacattatgactttggttactatctctttgccattgtcctccatttgggctggtgtttctttgacccgttcttcctccaccgaaaactctccatccagaggtgctttgtggaaccgtttgaccgactctaccggtaccagatctaaccaaacctcttccaccgacaccgccgtcgctatgacctacccatctaacaagtcttctactgtctgttacgccgatcaatcttctgtcaagagacaatacgatccagaacaaggtcacggtatctctgttgaacacgatgtttctgtccactcctgtcaaagattg

SEQ ID NO: 172 - DNA, 1236 bp, Artificial Sequence
  Description of Artificial Sequence Synthetic polynucleotide
atggatggttcttctgctccatcttctccaactccagatccaaccttcgacagattcgccggtaacgtcactttcttcttggctgaccacatcaccactacctccgttccaatgccagtcttgaacgcctactacgacgaatccttgtgtactaccatgaactacggtgctcaattaggtgcttgtttagttatgttggttgtcgttgttgctttgaccccagctgctaagttggctagaagaccagcttctgctttgcatttggttggtttgttgttgtgtgctgttagatccggtttgttgtttgcttacttcgtctccccaatctctcacttttaccaagtttgggctggtgacttctctgccgtttccagaagatactgggacgcttctttggctgccaacactttagctttcccattggttgtcgtcgttgaagctgctttgatcaaccaagcttggaccatggttgctttctggccaagagccgctaaggccgctgcctgtgcttgttctgctgtcattgtcttgttgactattggtactagattggcctacactatcgtccaaaaccacgctattgttactgccgtcccaccagaacacttcttgtgggctattcaatggtccgctgttatgggtgctgtttccatcttctggttttgtgccgttttcaacgtcaagttggtctgtcacttagtcgctaacagaggtatcttgccatctatctctgttgttaacccaatggaagtcttggttatgactaacggtaccttgatgattatcccatctatcttcgctggtttggaatgggctaagttcaccaacttcgaatccggttctttgactttgacttccgttattattatcttgccattgggtactttggctgcccaacgtatttctggtcaaggttcccaaggttaccaagctggtcacttattccacgaacaacaacaacaacaagctcgtacccgttccggtgccttcggttccgcttctcaacaatcccatccaactaacaaggttccatcctctattaccttgtctacctctggtactccaattactccacaaatctctgccggttcccgtccagaattaccattggttgatagatccgaacgtttggacccaattgacttggaattgggtagaatcgatgctttcagaggttcttccgacttctctccatccaccgctagaccaaagcgtatgcaacgtgataacttcgcc

SEQ ID NO: 173 - DNA, 1197 bp, Artificial Sequence
  Description of Artificial Sequence Synthetic polynucleotide
atggcttctaactcttctaacttcgacccattgactcaatctatcactatcttgatggctgacggtatcactactgtttctttcactccattggacatcgacttcttctactactacaacgttgcttgttgtatcaactacggtgctcaagctggtgcttgtttgttgatgttcttcgttgttgttgttttgactaaggctgttaagagaaagactttgttgttcgttttgaacgttttgtctttgatcttcggtttcttgagagctatgttgtacgctatctacttcttgcaaggtttcaacgacttctacgctgctttcactttcgacttctctagagttccaagatcttcttacgcttcttctgttgctggttctgttatcccattgtgtatgactatcactgttaacatgtctttgtacttgcaagcttacactgtttgtaagaacttggacgacatcaagagaatcatcttgactactttgtctgctatcgttgctttgttggctatcggtttcagattcgctgctactgttgttaactctgttgctatcttggctacttctgcttcttctgttccaatgcaatggttggttaagggtactttggttactgaaactatctctatctggttcttctctttgatcttcactggtaagttggtttggactttgtacaacagaagaagaaacggttggagacaatggtctgctgttagaatcttggctgctatgggtggttgtactatggttatcccatctatcttcgctatcttggaatacgttactccagtttctttcccagaagctggttctatcgctttgacttctgttgctttgttgttgccaatctcttctttgtgggctggtatggttactgacgaagaaacttctgctatcgacgtttctaacttgactggttctagaactatgttgggttctcaatctggtaacttctctagaaagactcacgcttctgacatcactgctcaatcttctcacttggacttctcttctagaaagggttctaacgctactatgatgagaaagggttctaacgctatggaccaagttactactatcgactgtgttgttgaagacaaccaagctaacagaggtttgagagactctactgaaatggacttggaagctatgggtgttagagttaacaagtcttacggtgttcaaaaggcttag

SEQ ID NO: 174 - DNA, 1410 bp, Artificial Sequence
  Description of Artificial Sequence Synthetic polynucleotide
atgaatatcaattcaactttcatacctgataaaccaggcgatataattattagttattcaattccaggattagatcaaccaattcaaattcctttccattcattagattcatttcaaaccgatcaagctaaaatagctttagtcatggggataactattgggagttgttcaatgacattaatttttttgatttctataatgtataaaactaataaattaacaaatttaaaattaaaattaaaattaaaatatatcttgcaatggataaatcaaaaaatcttcaccaaaaaaaggaatgacaacaaacaacaacaacaacaacaacaacaacaaattgaatcatcatcatataacaatactactactacgctggggggttataaattatttttattttatcttaattcattgattttattaattggtattattcgatcaggttgttatttaaattataatttaggtccattaaattcacttagttttgtatttactggttggtatgatggatcatcatttatatcatccgatgtaactaatggatttaaatgtattttatatgctttagtggaaatttcattaggtttccaagtttatgtgatgttcaaaacttcaaatttaaaaatttgggggataatggcatcattattatcaattggtttaggattgattgttgttgcctttcaaatcaatttaacaattttatctcatattcgattttcccgggctatatcaactaacagaagtgaagaagaatcatcatcatcattatcatctgattcggttgggtatgtgattaattcaatatggatggatttaccaacaatattattttccattagtattaatataatgacaatattattgattggtaaacttataattgctattagaacaagacgttatttaggattgaaacaatttgatagtttccatattttattaattggtttcagtcaaacattaattattccttcaattattttggtggttcattatttttatttatcacaaaataaagattctttattacaacaaattagtcttttattgattattttaatgttaccattaagttctttatgggctcaaactgctaataatactcataatattaattcatctccaagtttatcattcatatctcgtcatcatctgtctgatagtagtcgtagtggtggttccaatacaattgttagtaatggtggtagtaatggtggtggtggtggtggtgggaatttccctgtttcaggtattgatgcacaattaccacctgatattgaaaaaatcttacatgaagataataattataaattacttaatagtaataatgaaagtgtaaatgatggagatattatcattaatgatgaaggtatgattactaaacaaatcaccatcaaaagagtgtag

SEQ ID NO: 175 - DNA, 1128 bp, Artificial Sequence
  Description of Artificial Sequence Synthetic polynucleotide
atggaattcactggtgacatcgttttgaagtacactttgggtggtgaagaatacttgtctactttcgaacaattggactcttctgttaacagatctttggaattgggtgttgttcacggtatcgctatcgcttgtggtgttttgttgatggttttggcttgggttatcatcatcaagaagaagaacccaatcttcgttttgaaccaattaactttactattgatggttatcaagtcttctttatacttggctttcttgttcggtccattgtcttctttgacttacaagttcactagagttttgccacacgacaagtggcacgctttccacgtttacatcgctactaacgttatccacactttattgatcgctactgttgaaatgactttggtcttccaaatctacatcattttcaagtctccagaagttagacacttgggttacatcttgactggtgctgcttctgctttggctctaactatcgttgctttgtacatccactctactgttatctctgctgttcaattaaaggaacaattgttgatgcacgaaatcaagatcactaactcttgggttaacaacgttccaatcattttgttctcagcttctttgaacgttgtttgtatcattttgatcgctaagttagctttggctatcaagactagaagatacttaggtttgaagcaattcgacggtttgcacatcttgatgatcacttctactcaaactttcatcgttccatctgttttgatgatcgttaactacaagcaatcttcttcttacttgactttgttggctaacatctctgttatcttggttgtctgtaacttgccattgtcttctttgtgggctgcttctgctaacaattcttctactccaacttcttctgctaacactgttttctctagatgggactctaagttctctgacactgaaactatcgctcacgaattaccattgatcccaggtaaggctgaaaagttgcaattggtttctccaatcactgaaaagggtgacactcacactatgtgtgaatctcacggtgaccaagacttgatcgacaagatgttggacgacatcgaaggtgctgttatgactactgaattcaacttgaacaacagaactgtt

SEQ ID NO: 176 - DNA, 1371 bp, Artificial Sequence
  Description of Artificial Sequence Synthetic polynucleotide
atggctgctagaattatcccagctttgaccttgaccgccccaacctcttacccaaccgccggtgttggtggttactactacgacactgctttcggtgttccaacctactcctctgccgctttcaaccaaaccacctggagattgttggataactgggaccacatcaacgtcaactacgcttcttccgaaggtttggctgctggtttaggttgggctaccttgatttacttgttggctttgactccatcccacaagagaactactccattccactgtttcttgttggttggtttgattttcttgttgggtcacttgatggtcaacattattgccgccttgaccccaggtttgaacaccacctctgcttacacttacgttaccttggatacctcctcttccgtctggccacgtaagtacatcgctgtctacgctgtcaacgctgtcgcttcttggttcgctttcatttttgccactatctgtttgtggttgcaagctaaaggtttaatgaccggtatcagagtccgtttcatcatcgtctacaagattatcttgatgtacttgatcgttgctgctgtcattgctttggctatctgtatggctttcaacattcaacaaatcttatacattggtaagccagttgaattggctgacggtaccgctttgttgagattgagaaacgcttacttaatcacctacgctatctctattggttctttctccttagtttctatctgttctatcatggatatcatctggagaagaccatctagagtcattaagggtcacaacattttcgcttccgctttgaacttagttggtttgttgtgtgctcaatccttcgtcgtcccatgtgaatacaagagagccttgggtcaagtcccagattgtactactttcgccgatcacattttccacaccgttatcttctgtattttgcaagttattccaaactcttctggtgttatgttgccagaaatcatgttattgccatctgtttacgtcattttgccattgggttccttgttcatgactgttaactccccagaatccgatgtcaacaagacctctttcccaccaaagtcctccccaggtccattcgacagatccccaactttgacctctggtaccttgccaggttctagaccagaatcctacgttttggatatggcttctgacaagaactccggtaacagaaagtctgtttgttcccaattcgaccgtgaattgaacttgatcgattctttggacactttgtctggtcgtgaaggtgattctatgttgcacgcccaatccaacaacaacaaccaaaccagagaacaagacaagcaaccaagagccgataccacccacgttggttctgaaaacatggtc

SEQ ID NO: 177 - DNA, 1254 bp, Artificial Sequence
  Description of Artificial Sequence Synthetic polynucleotide
atggaaatgggttacgacccaagaatgtacaacccaagaaacgaatacttgaacttcacttctgtttacgacgttaacgacactatcagattctctactttggacgctatcgttaagggtttgttgagaatcgctatcgttcacggtgttagattgggtgctatcttcatgactttgatcatcatgttcatctcttctaacacttggaagaagccaatcttcatcatcaacatggtttctttgatgttggttatgatccactctgctttgtctttccactacttgttgtctaactactcttctatctcttacatcttgactggtttcccacaattgatcacttctaacaacaagagaatccaagacgctgcttctatcgttcaagttttgttggttgctgctatcgaagcttctttggttttccaaatccacgttatgttcactatcgaaaacatcaagttgatcagagaaatcgttttgtctatctctatcgctatgggtttggctactgttgctacttacttggctgctgctatcaagttgatcagaggtttgcacgacgaagttatgccacaaactcacttgatcttcaacttgtctatcatcttattggcttcttctatcaacttcatgactttcatcttagttatcaagttgttcttcgctatcagatctagaagatacttaggtttgagacaattcgacgctttccacatcttgttgatcatgttctgtcaatctttgttgatcccatctgttttgtacatcatcgtttacgctgttgactctagatctaaccaagactacttgatcccaatcgctaacttgttcgttgttttgtctttgccattgtcttctatctgggctaacacttctaacaactcttctagatctccaaagtactggaagaactctcaaactaacaagtctaacggttctttcgtttcttctatctctgttaactctgactctcaaaacccattgtacaagaagatcgttagattcacttctaagggtgacactactagatctatcgtttctgactctactttggctgaagttggtaagtactctatgcaagacgtttctaactctaacttcgaatgtagagacttggacttcgaaaaggttaagcacacttgtgaaaacttcggtagaatctctgaaacttactctgaattgtctactttggacactactgctttgaacgaaactagattgttctggaagcaacaatctcaatgtgacaagtag

SEQ ID NO: 178 - DNA, 1185 bp, Artificial Sequence
  Description of Artificial Sequence Synthetic polynucleotide
atgaagtcctgctccatcggtttcggtatcccattcattaatgaaccaaacttcgaaactgtttctattttgaccatggacgtttctttcattgacgctgacgtcaatcctgacaatatcttgttgaacttcaccattcctggttaccaaaacggtttctctgttccaatggttgttattaacgaattgcaaaagtctcaaatgaaatacgctattgtttacggttgtggtgtcggtgcctccttgattttgttgtttgtcgtctggattttgtgttctagaaagactccattgtttatcatgaacaacattccattagttttgtacgtcatctcctcttctttgaacttggcttacattaccggtccattgtcttctgtttccgtcttcttgaccggtatcttgacttctcacgatgccattaacgtcgtttacgcttccaacgctttgcaaatgttgttgatcttttctatccaatctaccatggcctaccacgtttacgttatgttcaaatctccacaaattaaatacttgagatacatgttagtcggtttcttgggttgtttacaaattgtcaccacctgtttatacatcaactacaatgttttgtactctcgtagaatgcacaaattgtacgaaactggtcaaacctaccaagatggtaccgttatgactttcgttccattcatcttgttccaatgttctgtcaacttctcttctattttcttggttttgaagttgattatggccattagaaccagacgttacttgggtttgcgtcaattcggtggttttcatattttgatgatcgtttctttacaaactatgttggtcccatctattttggttttggttaactacgccgctcataaggctgttccttccaacttgttatcttccgtttctatgatgatcattgttttgtctttaccagcttcttctatgtgggccgctgctgctaacgcctcttctgccccttcctccgctgcttcctccttgttcagatacaccacttctgattccgatagaactttggaaactaaatctgaccacttcatcatgaagcatgagtcccacaactcttctccaaattcctccccattgactttggttcaaaagagaatttctgatgccaccttagaattaccaaaagagttagaagacttgatcgactccacctccatc

SEQ ID NO: 179 - DNA, 1080 bp, Artificial Sequence
  Description of Artificial Sequence Synthetic polynucleotide
atgaacccagctgacatcaacatcgaatacaccttgggtgatactgctttctcttccactttcgctgatttcgaagcttggaaaactagaaacactcaattcgctattgtcaacggtgtcgctttggcttgtggtattatcttgatggtcgtttcttggattattattgttaacaagagagctccaatcttcgctatgaaccaaactatgttggttatcatggttattaagtccgctatgtacttgaagcatatcatgggtccattgaactccttgaccttccgtttcaccggtttaatggaagaatcctgggctccatacaacgtttacgtcactattaacgtcttgcatgttttgttggtcgctgctgtcgaatcctctttggtcttccaaatccatgttgttttcaagtcttctagagccagagttgctggtagagccattgtttctgctatgtccactttggccttgttgatcgtttctttgtacttgtactctactgttagacatgctcaaactttgcgtgctgaattatctcatggtgacactaccactgttgaaccatgggtcgataacgttccattgattttgttttccgcttctttgaacgttttgtgtttgttgttggccttgaaattggttttcgctgtcagaaccagaagacatttaggtttaagacaattcgactctttccacatcttgattattatggccactcaaactttcgttatcccatcctctttggtcatcgctaactacagatacgcttcttccccattgttgtcttccatttccatcatcgtcgccgtctgtaacttgccattgtgttccttgtgggcttgttctaacaacaactcttcctacccaacttcttctcaaaacactattttgtccagatacgaaactgaaacctctcaagctactgacgcttcctctaccacctgtgccggtattgctgaaaagggtttcgacaagtctccagactctccaactttcggtgaccaagactccgtctctatctcccatatcttggactctttggaaaaggatgttgaaggtgtcaccacccatagattgact

SEQ ID NO: 180 - DNA, 1257 bp, Artificial Sequence
  Description of Artificial Sequence Synthetic polynucleotide
atggactcctacttgttgaaccatccaggtgacatctctttgaacttcgccttgccattgtccgatgaagtctacactattaccttcaacgacttagactctcaatcttctttttccattcaatacttggtcatccactcttgtgccattaccgtctgtttgaccttgttggttttgttgaacttgttcatcagaaacaagaagactccagtcttcgttttgaaccaagtcatcttgttcttcgctatcgtcagatcttctttgttcatcggttttatgaagtctccattgtccaccatcaccgcctctttcaccggtatcatttctgatgaccaaaaacacttctacaaggtctccgtcgctgctaacgccgctttgatcattttggtcatgttgattcaagtttctttcacttaccaaatctacattattttcagatccccagaagttagaaagttcggtgtcttcatgacctccgccttgggtgtcttgatggctgttaccttcggtttttacgttaactccgctgtcgcttctaccaagcaataccaacacatcttctactctaccgacccatacatcatggactcttgggtcactggtttgccaccaatcttgtactctgcttccgtcatcgctatgtctttggtcttggttttgaagttggtcgctgctgtcagaaccagaagatacttgggtttgaagcaattctcctcctaccacatcttgttgattatgttcacccaaaccttgttcgttccaaccatcttgaccatcttagcttacgctttctacggttacaacgatatcttgatccatatttctaccaccatcaccgttgtcttgttgccattcacctccatttgggcttctatcgccaacaactctagatccttgatgtctgccgcttccttgtacttctccggttccaactcctctttgtctgaattgtcttctccatctccatctgataacgacactttgaacgaaaacgtcttcgccttttttccagacaagttgcaaaagatgaactcttctgaagccgtttctgctgtcgacaaggtcgttgttcacgaccactttgataccatctcccaaaagtctatcccacacgacatcttggaaattttgcaaggtaacgaaggtggtcaaatgaaggaacacatctctgtctactctgatgactctttctccaagactactccaccaattgtcggtggtaacttgttgatcaccaacaccgacatcggtatgaag

SEQ ID NO: 181 - DNA, 1209 bp, Artificial Sequence
  Description of Artificial Sequence Synthetic polynucleotide
atgaacaagattgtctccaagttgtcttcttctgacgtcatcgttaccgtcaccatcccaaacgaagaagatggtacttacgaagtcccattctacgctattgacaactaccactactcccgtatggaaaacgctgttgttttaggtgctaccattggtgcttgttctatgttgttgatcatgttgattggtattttgttcaagaacttccaaagattgagaaagtctttgttgttcaacatcaacttcgctatcttattgatgttgattttgagatccgcttgttacatcaactacttgatgaacaacttgtcttccatttctttcttcttcaccggtattttcgatgatgaatctttcatgtcttccgacgctgccaacgccttcaaggttatcttggttgccttgattgaagtttccttgacctaccaaatttacgttatgttcaagaccccaatgttgaagtcctggggtattttcgcctctgtcttggccggtgttttgggtttggctactttggctacccaaatctacactaccgttatgtctcacgttaacttcgtcaacggtaccaccggttctccatctcaagttacttccgcttggatggacatgccaactatcttattctccgtttctattaacgttttgtctatgttcttggtttgtaagttgggtttggccatcagaaccagacgttacttgggtttaaagcaattcgacgctttccacattttattcattatgtccactcaaaccatgatcattccatccatcatcttgttcgttcactacttcgatcaaaacgactctcaaaccaccttggtcaacatctctttgttattggtcgtcatttccttgccattgtcttctttgtgggctcaaactgctaacaacgttagaagaattgacacttctccatccatgtccttcatctctagagaagcttccaacagatctggtaacgaaaccttgcactctggtgctactatctctaagtacaacacctccaacaccgttaacactaccccaggtacttctaaggatgactctttgttcatcttggacagatccattccagaacaaagaattgtcgacactggtttgccaaaggacttggaaaagttcattaacaacgatttttacgaagacgatggtggtatgattgccagagaagtcaccatgttgaagaccgctcacaacaaccaa

SEQ ID NO: 182 - DNA, 1236 bp, Artificial Sequence
  Description of Artificial Sequence Synthetic polynucleotide
atggacatcaacaacaccatccaatcttccggtgacatcatcattacctacaccatcccaggtatcgaagaaccattcgaattgccattcgaagttttgaaccacttccaatctgaacaatccaagaactgtttggtcatgggtgttatgatcggttcttgttccgttttgttgatcttcttggtcggtattttgttcaaaaccaacaaattctctactattggtaagtctaagaacttgtctaagaacttcttgttctacttgaactgtttgatcaccttcatcggtatcattcgtgctgcctgtttttctaactacttgttgggtccattgaactctgcttctttcgctttcactggttggtacaacggtgaatcttacgcttcttccgaagctgctaacggtttcagagtcatcttgttcgctttgattgaaacttctatggtcttccaagttttcgttatgttcagaggtgctggtatgaaaaagttggcttactccgttaccattttgtgtaccgctttggctttggtcgttgttggtttccaaattaactccgctgtcttatctcacagaagattcgtcaacaccgttaacgaaattggtgatactggtttgtcctccatttggttggacttgccaaccatcttgttctccgtctctgtcaacttaatgtctgttttgttgatcggtaaattgatcatggctattaagactagaagatacttgggtttgaaacaattcgattccttccacgttttgttaatttgttccactcaaactttgttggtcccatctttaatcttgttcgttcactacttcttgttctttagaaacgccaacgttatgttgattaacatttccatcttgttgatcgtcttgatgttgccattctcttccttgtgggctcaaaccgccaacaccacccaatacatcaactcttccccatccttctctttcatctctagagaaccatctgctaactctactttgcactcctcttccggtcactactctgaaaagtcctacggtattaacaaattgaacacccaaggttcttccccagccaccttaaaggatgatcacaactccgtcatcttggaagctaccaacccaatgtctggtttcgacgcccaattgccaccagacattgctagattcttgcaagatgacatcagaattgaaccatcttctacccaagatttcgtttccactgaagtcacctacaagaaggtc

SEQ ID NO: 183 - DNA, 1581 bp, Artificial Sequence
  Description of Artificial Sequence Synthetic polynucleotide
atggaccacaacacccaacacttcaacagacctgaatacattgaaatcccagttccaccatctaagggtttcaacccacacaccaaccctgctttcttcatctacccagacggttctaatatgaccttttggttcggtcaaatcgacgatttcagacgtgaccaattattcactaacaccatcttttccattcaaattggtgccgctttggtcatcttatgtgtcatgttttgtgttacccacgctgataagcgtaaaaccattgtctacttgttaaacgtttccaacttgttcgttgttatcattagaggtgttttctttgttcattacttcatgggtggtttggccagaacctataccactttcacctgggatacttctgatgttcaacaatctgagaaggctacttccattgtctcctctatttgttctttgattttgatgatcggtactcaaatctccttattgttgcaagtcagaatctgttacgctttgaacccaagatccaagaccgctatcttggttacttgtggttctatttccggtattgctaccactgcttatttattgttgggtgcttacactattcaattgagagaaaagccaccagacatgaagttcatgaagtgggctaagccagttgttaacgctttggttgccttgtccattgtctccttttctggtattttctcttggagaatgttccaatctgtcagaaacagaagaagaatgggtttcactggtatcggttccttggaatctttgttggcttctggtttccaatgtttagtcttccctggtttggttactaccgctttgaccgtcgccggttccacttggtatatcgctgttaacttaactactccatctgacttgaccgctatttacaactgttccgcttttttcgcttatgctttctccattccattgttaaaggaaagagctcaagttgaaaagaccatttctgttgtcattgctatcgctggtgtcttagtcgttgcttacggtgacggtgctgacgacggttccacctctaacggtgaaaaggctagattgggtggtaacgtcttgatcggtatcggttctgtcttgtatggtttatacgaagtcttgtataagaagttattatgtccaccatctggtgcttccccaggtagatctgttgttttctctaataccgtttgtgcttgcatcggtgctttcactttgttattcttgtggatcccattgccattgttgcactggtccggttgggaaatttttgaattgccaaccggtaagactgctaagttattgggtatttccattgccgctaacgccaccttctctggttctttcttgatcttaatttctttgactggtccagttttgtcctctgttgccgccttgttgaccattttcttggttgctattactgacagaattttattcggtagagaattgacttctgctgccattttgggtggtttgttgatcatcgctgccttcgctttgttatcttgggctacttggaaggaaatgattgaagagaacgagaaggatactatcgattccatctctgacgttggtgaccacgatgac

SEQ ID NO: 184 - DNA, 1161 bp, Artificial Sequence
  Description of Artificial Sequence Synthetic polynucleotide
atgtctaaggaagttttcgacccattcactcaaaacgttactttcttcgctccagacggtaagactgaaatctctatcccagttgctgctatcgaccaagttagaagaatgatggttaacactactatcaactacgctactcaattgggtgcttgtttgatcatgttggttgttttgttggttatggttccaaaggaaaagttcagaagaccattcatgatcttgcaaatcacttctttggttatctcttgttgtagaatgttgttgttgtctatcttccactcttctcaattcttggacttctacgttttctggggtgacgaccactctagaatcccaagatctgcttacgctccatctgttgctggtaacactatgtctttgtgtttggttatctctgttgaaactatgttgatgtctcaagcttggactatggttagattgtggccaaacgtttggaagtacatcatcgctggtgtttctttgatcgtttctatcatggctatctctgttagattggcttacactatcatccaaaacaacgctgttttgaagttggaaccagctttccacatgttctggttgatcaagtggactgttatcatgaacgttgcttctatctcttggtggtgtgctatcttcaacatcaagttggtttggcacttgatctctaacagaggtatcttgccatcttacaagactttcactccaatggaagttttgatcatgactaacggtatcttgatgatcatcccagttatcttcgcttctttggaatgggctcacttcgttaacttcgaatctgcttctttgactttgacttctgttgctgttatcttgccattgggtactttggctgctcaaagaatcgcttcttctgctccatcttctgctaactctactggtgcttcttctggtatcagatacggtgtttctggtccatcttctttcactggtttcaaggctccatctttctctactggtactactgacagaccacacgtttctatctacgctagatgtgaagctggtacttcttctagagaacacatcaacccacaaggtgttgaattggctaagttggacccagaaactgaccaccacgttagagttgacagagctttcttgcaaagagaagaaagaatcagagctccattgtag

SEQ ID NO: 185 - DNA, 1305 bp, Artificial Sequence
  Description of Artificial Sequence Synthetic polynucleotide
atggccgaagactccatcttcccaaacaactccacctctccattgaccaacccaattgttgttgaaaccattaagggtaccgcttacattccattacactacttggatgatttgcaatacgaaaagatgttgttggcttccttgttctccgttagaattgctacttccttcgttgttattatttggtacttcgtcgctgtcaacaaggctaagagatctaagtttttgtacattgtcaaccaagtttctttgttgatcgtttttatccaatccattttgtctttgatttacgtcttctccaacttctccaagatgtctaccattttgaccggtgattacaccggtatcactaagagagacattaacgtctcttgtgttgcctccgttttccaattcttgttcatcgcttgtatcgaattggctttgttcatccaagctactgtcgttttccaaaaatctgttagatggttgaagttttccgtttctttgatccaaggttccgtcgctttgactactaccgccttgtacatggccattattgtccaatccatctacgctactttgaacccatacgctggtaacttgattaaaggtcgtttcggttacttattagcttctttgggtaagattttcttctctatttctgttacttcttgtatgtgtatcttcgttggtaagttggtctttgctattcaccaaagaagaactttgggtattaagcaattcgacggtttgcaaattttggtcattatgtctactcaatccatgatcatcccaactattatcgtcttgatgtcttttttgagacgtaacgctggttctgtttacaccatggctaccttgttggtcgctttgtccttgccattgtcctccttgtgggctgaagccaagactaccagagactctgcttcttacaccgcttacagaccatctggttctccaaacaaccgttctttgttcgccatcttctctgatagattggcttgtggttctggtagaaacaacagacacgatgatgattctagaggtaacggttctgttaacgccagaaaggctgacgtcgaatctactatcgaaatgtcctcttgttacactgattccccaacctactccaagttcgaagctggtttggacgctagaggtatcgtcttctacaacgaacacggtttgccagttgtctccggtgaagttggtggttcttcctccaacggtactaagttgggttctggtcataagtacgaagtcaacactactgttgttttgtctgatgttgactctccatctccaaccgacgtcacccgtaag

SEQ ID NO: 186 - DNA, 888 bp, Artificial Sequence
  Description of Artificial Sequence Synthetic polynucleotide
atgtcttccttcgacccatacactcaaaacattactattttggtttctccatcctctccaccaatttccattccaatcccagttatcgacgctttcaacgacgaaaccgcttctatcattactaactacgccgctcaattaggtgctgctttggccatgttattagttttgttggccgctactccaaccgctagattgttaagagctgatggtccatccttgttgcacgctttggccttgttagtctgtgtcgtcagaactgtcttattgatctacttcttcttgaccccattctctcacttctaccaagtctggaccggtgacttctctcaagttccagcttggaactacagagcttctattgctggtaccgttttgtctactttgttgaccgttgttaccgacgctgctttggttaaccaagcttggactatggtttctttattcgctccaagaactaagagagccgtttgtgttttgtccttgttaatcaccttgttggccatttctttcagagtcgcttacaccgtcattcaatgtgaaggtatcgctgaattggctgctccaagacaatacgcttggttgatcagagccactttgatctttaacatctgttccattgcctggttctgtgctttgttcaactctaagttggttgctcacttggttaccaacagaggtgtcttgccatcccgtagagccatgtccccaatggaagttttgattatggccaacggtatcttgatgattgttccagttgttttcgctatcttggaatggcaccacttcattaacttcgaagctggttctttaaccccaacctccatcgccattatcttgccattgtcctctttggccgcccaaagaatcgccaacacttcttcctct

SEQ ID NO: 187 - DNA, 1308 bp, Artificial Sequence
  Description of Artificial Sequence Synthetic polynucleotide
atgtcagaagagatacccagtttgaacccattgttctacaatgagacatataatccattgcagtccgtcctaacatacagttcaatttacggagatgggactgaaataacatttcaacagctacaaaatcttgtccatgaaaacatcacccaagcaattatttttggaacaaggatcggcgctgctggattagcgttgattataatgtggatggtctctaagaatagaaagacgccgatattcataataaatcagagttctttggttcttacaattgttcaatctgctttatatctatcatatttgttgagcaattttggaggagttccctttgctctaactttgttcccacagatgataggcgaccgtgacaaacatctttacggtgccgtgactctaattcaatgtctattggttgcgtgtattgaggtctcgttagtctttcaggtaagagtcattttcaaagcagatagatataggaagataggaatcattttgactggcgtctccgctagttttggtgctgcaactgtagccatgtggatgattactgcaataaaatctattattgtagtgtatgatagtccattgaacaaagttgacacatattattacaacatagcagttattttacttgcatgttcaataaatttcatcactcttcttctatcagtgaaacttttcctggctttcagagctaggagacatttaggtttgaaacaatttgactcatttcacattctactcatcatgtctactcagacattaataggtccatcggttttgtatattctcgcctacgcgctgaacaataaaggagttaagtcgttgacttctattgctacattgcttgtagttctttccctacctttgacatctatctgggctgctgctgcaaatgatgcaccaagtgccagtactttctatcgccaattcaacccttactctgcacaaaatcgtgatgattcatcatcctactcttatggtaaagcctttagtgacaaatactctttcagtaactcaccacaaacttcggatggttgtagttcaaaggaacttgaactatctacacagttggagatggatttagagtctggcgaatcttttatggatagagcaaaaaggtccgattttgtttcttctccaggatcaacagatgcaacagtgattaaacaattgaaagcttccaacatctatacctcagaaacagatgctgatgaagaggcaagggcattttgggtgaatgcaattcatgaaaacaaagatgacggtttaatgcaatcgaaaaccgtattcaaagaattaaga

SEQ ID NO: 188 - DNA, 1062 bp, Artificial Sequence
  Description of Artificial Sequence Synthetic polynucleotide
atggaagaatactccgactccttcgacccatcccaacaattgttgaacttcacttccttatacggtgaaaccgatgctactttcgctgaattggacgactaccacttctacgtcgttaagtacgccatcgtttacggtgccagaattggtgtcggtatgttttgtactttgatgttgttcgttgtttccaagtcttggaagactccaatcttcgtcttgaaccaatcttctttgattttgttgattattcactccggtttctacatccactacttgaccaaccaattctcttccttgacctacatgttcactagaatcccaaacgaaacccatgctggtgtcgatttgcgtattaacgtcgttaccaacaccttgtacgctttgttgatcttatctattgaaatttccttaatttaccaagtcttcgttatcttcaaaggtgtctacgaaaactctttaagatggattgttactattttcaccgctttattcgccgccgccgtcgttgctattaacttctacgtcactactttgcaatctgtctctatgtacaactctaacgttgactttccaagatgggcttctaacgtcccattgatcttgttcgcttcttctgtcaactgggcttgtttgttgttgtccttgaagttgttcttcgctatcaaggttagaagatctttgggtttgagacaattcgacacttttcacatcttggccatcatgttctctcaaactttgattatcccatccattttgattgtcttgggttacactggtaccagagacagagactccttggcttctttgggtttcttgttgatcgttgtttctttgccattttcctctatgtgggctgccactgctaacaactccaacatcccaacctctaccggttctttcgcctggaagaacagatactccccatctacttactccgacgataccactgctgtttccaagtccttcactattatgaccgctaaggatgaatgtttcaccactgataccgaaggttctccaagattcatcaagggtgacagaacctccgaagatttgcacttc

SEQ ID NO: 189 - DNA, 1308 bp, Artificial Sequence
  Description of Artificial Sequence Synthetic polynucleotide
atggacgaagcaatcaatgcaaaccttgtttctggagatattatagtctcttttaacattcctggtttgccagaaccggtacaagtgccattcagcgaatttgattcgtttcataaagaccagctcattggagtcatcattcttggagtcactattggagcatgctcgcttttgttgatattgctacttggaatgttatacaagagccgtgaaaagtattggaaatcactattatttatgctcaatgtatgcatcttggctgccacaatcttaaggagcggttgcttcttagactattatctaagtgatttggccagtatcagttatacatttactggagtatacaatggtaccagctttgctagctctgacgcggcaaatgtgttcaagactattatgtttgccttgattgaaacttcgttaacctttcaagtgtatgtcatgtttcaagggaccacttggaaaaattggggccatgctgtcactgcattatcgggtctcttgtctgttgcctcagtggcgttccagatctacaccacgattttatcccacaataatttcaatgctacaatctcgggaaccggtacattaacttcaggtgtttggatggacttaccaacactcttgtttgccgcaagtatcaattttatgaccattttgttgttatttaagttgggaatggccattagacaaagaaggtatttaggtttaaaacagtttgatgggttccatatcttattcatcatgtttacccaaacattgttcataccctcgattttgcttgtgatccactacttttaccaggcaatgtctggaccattcatcatcaacatggcgttgttcttggtggtggcattcttgccattgagttcattatgggcacaaactgcaaacactactaaaaagattgaatcttcgccaagtatgagctttattactagacgaaaatcagaggatgagtcaccactggctgctaacgacgaggataggttacgaaaattcaccacaactttggatttgtcgggcaacaagaacaatacaacaaacaataataacaatagcaacaacattaacaacaatatgagcaacatcaactacccttctacaggactgggagaagacgataaatcctttatatttgagatggaacccagtcgggaaagagctgcaatagaagagattgatcttggagcaaggatcgataccggtttgcccagagatttagagaaatttctagttgatgggtttgacgatagtgatgacggagaaggaatgatagccagagaagtgactatgttgaaaaaatag

SEQ ID NO: 190 - DNA, 1266 bp, Artificial Sequence
  Description of Artificial Sequence Synthetic polynucleotide
atggtggtaacagctccaccttcagttgacagaacatattttatcccgaattctacctttgatccatatcaacaagacttgacgttggtctatcccgatggtgtgcacgccctggttgctaacgttgatgatatagtgtacttcatgggtctagcagttaagtctacgctaatatttgctattcaaattggtatttcatttgtattaatgttggttattgccctgttgacgaaacctgaaagaagagttacgttggtattcttcttaaacatgactgcactttttaccatcttcatcagagccatattgatgtgtactacatttgttggtacatattacaatttttacaactggattatgggcaactacccgaactctggtttagctgatcgtgtatctattgcagccgaagtttttgcttttctgattatactgtcattagaactttctatgatgtttcaagttcgtattgtatgcatcaacctgagctcattcaggaggagaataattacttttagtagtatagtggttgcaatgattgtttgtacagttagatttgcccttatggtgttgtcttgtgattggaggattgtgaatatcggagatgcgacgcaagaaaagaacagaatcattaaccgtgtggcatccggttataacatatgcacaatagcatcaatcatttttttcaacaccatcttcgtctccaagttggccgtcgctatcaaacatcgtagaagcatgggcatgaaacaattcggtccaatgcagatcatctttgttatgggttgtcaaacgcttctaattccagccatctttggaattatatcttactttgctctagctagcactcaggtctactctttaatgccaatggtcgtagctatcttcttaccattaagttctatgtgggctagttttaacaccaacaaaaccaacagtgttacaaatatgaggcaaccaaacgtctataggcctaatatgatcatcggtcaagacacaacccaaaattccggaaagaatacaaacataagtggtacgtcaaactccacggcaactacaagtagttttgctagcgataagagacgtctaaatttatctttcaatacacaaggtacactggttaattcaataagtgaagaagaggttaataacccacaaaaattgggtccttccgctaccgttgcggtaatggatagagattctttggaattagagatgagacaacacggcatcgctcaaggtaggtcatactcagtccgttccgac

SEQ ID NO: 191 - DNA, 1248 bp, Artificial Sequence
  Description of Artificial Sequence Synthetic polynucleotide
atggaccaaactttgtctgctactggtactgctacttctccaccaggtccagctttgactgttgacccaagattccaaactatcactatgttgactccagctttgatgggtcaaggtttcgaagaagttcaaactactccagctgaaatcaacgacgtttacttcttggctttcaacactgctatcggttactctactcaaatcggtgcttgtttcatcatgttgttggttttgttgactatgactgctaaggctagattcgctagaatcccaactatcatcaacactgctgctttggttgtttctatcatcagatgtactttgttggttatcttcttcacttctactatgatggaattctacactatcttctctgacgacttctctttcgttcacccaaacgacatcagaagatctgttgctgctactgttttcgctccattgcaattggctttggttgaagctgctttgatggttcaagcttgggctatggttgaattgtggccaagagcttggaaggtttctggtatcgctttctctttgatcttggctactgttactgttgctttcaagtgtgcttctgctgctgttactgttaagtctgctttggaaccattggacccaagaccatacttgtggatcagacaaactgacttggctttcactactgctatggttacttggttctgtttcttgttcaacgttagattgatcatgcacatgtggcaaaacagatctatcttgccaactgttaagggtttgtctccaatggaagttttggttatggctaacggtttgttgatggttttcccagttttgttcgctggtttgtactacggtaacttcggtcaattcgaatctgcttctttgactatcacttctgttgttttggttttgccattgggtactttggttgctcaaagattggctgttaacaacactgttgctggttcttctgctaacactgacatggacgacaagttggctttcttgggtaacgctactactgttacttcttctgctgctggtttcgctggttcttctgcttctgctactagatctagattggcttctccaagacaaaactctcaattgtctacttctgtttctgctggtaagccaagagctgacccaatcgacttggaattgcaaagaatcgacgacgaagacgacgacttctctagatctggttctgctggtggtgttagagttgaaagatctatcgaaagaagagaagaaagattgtag

SEQ ID NO: 192 - DNA, 1698 bp, Artificial Sequence
  Description of Artificial Sequence Synthetic polynucleotide
atggcgtcctcttcctcaccacctgcagacattttctcagggatcacgcaatcactaaatagtacacacgcgacgcttacactaccgattccgccagcggacagggatcatctggaaaatcaagtattatttttgtttgacaatcacggtcagttacttaatgtaactacaacttacattgacgcttttaacaatatgctggtctctactactataaactatgcaacgcaaattggagctacttttataatgctagccattatgttattaatgactcccagaaggaggttcaaacgtttaccaacaattattagcttgttagccttatgtattaatttgatcagggtggttttgctggccctgttttttccttctcactggacagacttctacgtgttgtattccggtgactggcagtttgtacctccaggggatatgcaaatatctgttgctgctacggttttgtctatcccagtgacggcattattattgagcgcattgatggttcaagcctggtcaatgatgcaattatggacaccactgtggagggcactagtggtactagtgtccgggctattgtcactggtaactgtggcaatgagtttcgcgaattgcattttccaagcgaaaaatattttgtatgccgaccctttaccctcctactgggtcagaaaattgtacttagcattaacgactgggtctataagttggttcacattcctttttatgataagattggttatgcatatgtggacaaacagatctatattaccaagcatgaagggtttgaaggctatggatgtattgattattacgaattctatattgatgttaatcccagtgttgtttgcaggcttggaatttctggatagtgcctctggatttgagtccgggtctttgactcaaacctctgtagtgattgtcctgcctttgggtactttagtagcacaaagaatagctacgaggggttacatgcccgatagtctggaggcttctagcggaccaaatggttcattgccgttatctaatttaagtttcgctggagggggcggtggtggttctgggggacataaagataaagaaaacggtggcggtattataccgcctactacgaacaatactgctgctactaatttttcttcatcaatcgcgtgttctggtatatcttgtttaccaaaagtcaaaagaatgaccgcgagttcagcctcaagtagccagagaccgttgttgacaatgactaactcaaccatagcgagtaatgacagttcaggtttcccttctcctggcatacataataccactactacgacaacacaataccaatattccatgggaatgaacatgccgaactttcctccagtcccgttcccaggttaccagtcacgtactaccggtgttacttcccatattgtgtccgacggtagacatcaccagggtatgaacaggcacccatctgttgaccattttgatagggaacttgctaggattgatgatgaagatgacgatggttaccctttcgcatcaagtgaaaaggccgttatgcacggagacgatgacgacgatgtggaaaggggacgtcgtagagctctaccaccatccttaggtggagttagagttgaaaggacgatcgagaccaggagcgaggaacgtatgccatctccggacccattgggtgttacgaagcctagatcattcgag

SEQ ID NO: 193 - DNA, 1071 bp, Artificial Sequence
  Description of Artificial Sequence Synthetic polynucleotide
atggcaccctcattcgaccccttcaaccaaagcgtggtcttccacaaggccgacggaactccattcaacgtctcaatccatgaactagacgacttcgtgcagtacaacaccaaagtctgcatcaactactcttcccagctcggagcatctgtcattgcaggactcatgcttgccatgctgacacactcagaaaagcgtcgtctgccagttttcttcctaaacacattcgcactggccatgaactttgcccgcctgctctgcatgaccatctacttcaccacgggcttcaacaagtcctatgcctactttggtcaggattactcccaggtgcctgggagcgcctacgcagcctctgtcttgggcgttgtcttcaccactctcctggtaatcagcatggaaatgtccctcctgatccaaacaagggttgtctgcacgacccttccggatatccaacgttatctactcatggcagtttcctccgcgatttccctgatggccatcgggttccgccttggcttaatggttgagaactgcattgccattgtgcaggcgtcgaatttcgccccttttatctggcttcaaagcgcctcgaacatcaccattacgatcagcacatgtttcttcagtgccgtctttgttacgaaattggcatatgcactcgtcactcgtatacgactaggcttgacgaggtttggtgctatgcaggttatgttcatcatgtcctgccagactatggtgattccagccatcttctcaattctccaatacccactccccaagtacgaaatgaactccaacctctttacgctggtggccattttcctccctctttcctcgctatgggcttcagttgctacgagatccagtttcgagacgtcttcttccggccgccatcagtatctttggccaagcgaacagagcaataacgtcaccaattcggaaattaagtatcaggtcagcttctctcagaaccacactacgttgcggtctggagggtctgtggccacgacactctccccggaccggctcgacccggtttattgtgaagttgaagctggcacaaaggcctag

SEQ ID NO: 194 - DNA, 1191 bp, Artificial Sequence
  Description of Artificial Sequence Synthetic polynucleotide
atgtccactgccaacgttcatttaccagctgatttcgatccaactagacaaaacatcactatctataccccagacggtaccccagttgttgctaccttgccaatgatcaatttgtttaacagacaaaacaacgaaatctgtgttgtttacggttgtcaattgggtgcctctttaattatgttcttggttgttttgttgaccaccagagtttccaagagaaaatctccaatcttcgtcttgaacgttttgtctttgattatttcttgtttaagatccttgttgcaaattttatactatattggtccatggaccgagatctacagatacttgtctttcgattactctactgtcccagcttccgcttacgctaattctgttgctgccactttattaaccttattcttattgattaccattgaagcttctttagttttacaaactaacgttgtctgcaagtctatgtcttctcacattcgttggccagttactgctttgtccatggttgtctctttattggctatttcttttagattcggtttgaccatccgtaacatcgaaggtatcttaggtgctactgtcaaatccgactccttaatgttctctggtgcctctttgatctctgaaactgcttctatctggttcttctgcactattttcgttattaaattgggttggaccttgtaccaaagaaagaagatgggtttgaagcaatggggtccaatgcaaattatcactatcatggctggttgcaccatgttgatcccatccttgttcactgttttggaattcttccctgaagaaactttctacgaggccggtactttggctatctgtttggttgctattttgttgccattatcttccgtctgggctgccgctgctattgatggtgatgaaccagtccgtccacatggttctaccccaaaattcgcttctttcaacatgggttccgactacaaatcttcttctgctcacttgccaagatctattagaaaggcctccgtcccagctgaacatttatctagaacttctgaagaagagttaggtgacgacggtactttgaacagaggtggtgcctacggtatggacagaatgtccggttctatctcccctagaggtgtcagaattgaaagaacttacgaagttcataccgctggtagaggtggttctatcgagagagaggacatcttc

SEQ ID NO: 195 - DNA, 1140 bp, Artificial Sequence
  Description of Artificial Sequence Synthetic polynucleotide
atggctacctcttccccaatccaaccatttgacccattcacccaaaacgttaccttccgtttgcaagacggtaccgaattcccagtttctgtcaaggctttggacgtcttcgtcatgtacaacgttagagtctgtattaactacggttgtcaattcggtgcctccttcgtcttgttagtcattttagtcttgttaactcaatccgacaagagaagatctgctgtcttcattttgaacggtttggctttgttcttgaactcttctagattgttgtttcaagttattcacttctccactgccttcgaacaagtctacccatacgtctctggtgactactcctctgtcccatggtccgcttacgctatctccattgtcgctgttgttttgactaccttggtcgttgtttgtatcgaagcttctttggttattcaagttcacgttgtctgttccaccttgagacgtagatacagacacccattattagctatttctattttggtcgctttggttccaatcggtttcagatgtgcttggatggtcgctaactgtaaggctattattaaattgacctacaccaacgacgtttggtggatcgaatctgctactaacatctgtgtcactatctccatctgtttcttctgtgttatcttcgttaccaagttgggtttcgccatcaagcaaagaagaagattgggtgttagagaattcggtccaatgaaggttattttcgtcatgggttgtcaaactatggttgttccagctattttctccatcacccaatactacgtcgtcgtcccagaattctcctctaacgtcgttactttggttgtcatttctttaccattatcttccatttgggccggtgctgtcttggaaaacgctagaagaaccggttcccaagatagacaaagaagacgtaacttgtggagagctttggttggtggtgctgaatccttgttatccccaactaaggactctccaacctctttgtctgctatgactgctgctcaaaccttatgttactctgatcacaccatgtccaagggttctccaacttccagagacaccgatgctttctacggtatctccgttgaacacgacatctccattaacagagttcaacgtaacaactccatcgtc

SEQ ID NO: 196 - DNA, 1296 bp, Artificial Sequence
  Description of Artificial Sequence Synthetic polynucleotide
atgtctgatgcggctccttcattgagcaatctattttatgatccaacgtataatcctggtcaaagcaccattaactacacttccatatatgggaatggatctaccatcactttcgatgagttgcaaggtttagttaacagtactgttactcaggccattatgtttggtgtcagatgtggtgcagctgctttgactttgattgtcatgtggatgacatcgagaagcagaaaaacgccgattttcattatcaaccaagtttcattgtttttaatcattttgcattctgcactctattttaaatatttactgtctaattactcttcagtgacttacgctctcaccggatttcctcagttcatcagtagaggtgacgttcatgtttatggtgctacaaatataattcaagtccttcttgtggcttctattgagacttcactggtgtttcagataaaagttattttcacaggcgacaacttcaaaaggataggtttgatgctgacgtcgatatctttcactttagggattgctacagttaccatgtattttgtaagcgctgttaaaggtatgattgtgacttataatgatgttagtgccacccaagataaatacttcaatgcatccacaattttacttgcatcctcaataaactttatgtcatttgtcctggtagttaaattgattttagctattagatcaagaagattccttggtctcaagcagttcgatagtttccatattttactcataatgtcatgtcaatctttgttggttccatcgataatattcatcctcgcatacagtttgaaaccaaaccagggaacagatgtcttgactactgttgcaacattacttgctgtattgtctttaccattatcatcaatgtgggccacggctgctaataatgcatccaaaacaaacacaattacttcagactttacaacatccacagataggttttatccaggcacgctgtctagctttcaaactgatagtatcaacaacgatgctaaaagcagtctcagaagtagattatatgacctatatcctagaaggaaggaaacaacatcggataaacattcggaaagaacttttgtttctgagactgcagatgatatagagaaaaatcagttttatcagttgcccacacctacgagttcaaaaaatactaggataggaccgtttgctgatgcaagttacaaagagggagaagttgaacccgtcgacatgtacactcccgatacggcagctgatgaggaagccagaaagttctggactgaagataataataatttatag

SEQ ID NO: 197 - DNA, 1431 bp, Artificial Sequence
  Description of Artificial Sequence Synthetic polynucleotide
atgtctgacgctccaccaccattgtccgaattgttctacaactcctcctacaacccaggtttgtctatcatttcttacacttccatttacggtaacggtactgaagttacctttaacgaattacaatctatcgtcaacaagaagattactgaagctatcatgttcggtgtcagatgtggtgccgctattttgactatcattgtcatgtggatgatttctaagaagaaaaagaccccaattttcatcatcaaccaagtttctttattcttgattttgttgcactccgctttcaacttcagatacttgttgtctaactactcttccgtcactttcgccttgaccggtttcccacaattcatccacagaaacgacgtccacgtctacgctgctgcttctatcttccaagtcttgttggtcgcttctattgaaatttccttaatgttccaaatcagagtcattttcaagggtgataacttcaagagaattggtactatcttgaccgctttgtcctcttctttgggtttagctactgttgctatgtactttgtcaccgctattaagggtattattgctacctacaaggatgttaacgatactcaacaaaagtacttcaacgttgctactatcttgttggcttcctctatcaactttatgaccttgatcttggttatcaagttgatcttggctatcagatccagaagattcttgggtttgaaacaattcgactctttccatatcttgttgatcatgtcttttcaatctttgttggccccatccattttgttcattttggcttactctttggacccaaaccaaggtaccgacgtcttggttactgtcgctactttgttggtcgtcttatctttgccattgtcctccatgtgggctactgctgctaacaacgcctccagaccatcctctgttggttccgactggactccatctaactccgactactactctaacggtccatcttctgtcaagaccgaatctgtcaaatctgatgaaaaggtctccttgagatccagaatttacaacttgtacccaaagtctaagtctgaattcgaacaatcctccgaacacacttacgttgacaaggtcgacttggaaaacaacttctacgaattgtccaccccaatcaccgaaagatctccatcttctatcattaagaagggtaagcaaggtatttctactagagaaaccgtcaaaaagttggactccttggatgacatttacactccaaacactgctgctgatgaagaagccagaaagttctggtctgaagatgtttctaacgaattggattccttacaaaaaatcgaaactgaaacttccgatgaattatccccagaaatgttacaattgatgattggtcaagaagaagaagacgataacttattggctaccaagaagatcaccgtcaagaagcaa

SEQ ID NO: 198 - DNA, 1404 bp, Artificial Sequence
  Description of Artificial Sequence Synthetic polynucleotide
atgaaacccgccgctggacctgcatctagtccattcgacccatttaaccaaacgttttacctgaccggtccagataataccactgtaccagtctcagtcccacaagttgactatatctggcattatattattggaacatccatcaactatggttctcagatcggagcctgtttacttatgcttcttgtgatgttgacattgacttcaaagtcaagattttctcgtgcggccactctgattaacgtagcaagcttattgattggagtaattcgttgtgttcttttagctgtctactttacttcttctctaactgaattgtatgctctgttcgttggcgattacagccaggtccgtaggtctgatctttgtgtctctgctgtggcaaccttctttagtctaccacaattagttctaatagaagctgctttgtttctacaggcttatagtatgatcaaaatgtggccatccctgtggagagcagtggttttagctatgtcagtggtggtggctgtgtgtgcaatcggttttaagttcgcgtccgttgttatgcgtatgaggtcaacattaacattggacgattctttggatttctggctagtggaagtcgatctggcttttacagcaactactattttttggttttgtttcatctacattataaggttggttattcatatgtgggaatatagaagcattttaccaccaatggggtctgtttctgctatggaggttcttgttatgaccaatggagcgttgatgttagttccagtgattttcgccgcaatagaaatcaatggtttatcaagctttgaatcagggtcactggttcatacatcagtgattgtattattacctttaggtagcttgatagcgcaagcaatgacacgtccagatgggtatgtccaaagaacgaatacatctggagcatcaggcgcaagtggtgcacatcctggtagaaatggatccggacacggtggtcatggtggtgcgtactcaagagccatgactaataccctaaatacattggatacattggataccgtagacagtaagacatccataatgcatcatcatcatcaccatcatagaaaccactcaaatggcatgagtaagacgaaggcaaatagtggaacatggagccatgcgtcagatgctaactccaccaatgctatgatcagcggtggtatcgcaactcaagttaggattcaagctaatcagtcaaccttaggaaatacggggatgtccgggggctctggagcccctaattctcatactcgtaataactcattggctgctatggaaccagtggagaagcaactgcatgatatcgatgccacacctttaagcgcatctgattgcagggtctgggttgatcgtgaggtcgaggtcagaagggacatggtc

SEQ ID NO: 199 - DNA, 1038 bp, Artificial Sequence
  Description of Artificial Sequence Synthetic polynucleotide
atgtactcctgggacgaattcagatccccaaagcaagctgaagttttgaaccaaaccgttaccttggaaactattgtttccaccattcaattgccaatctctgaaattgactccatggaaagaaacagattgttgaccggtatgactgtcgctgttcaagttggtttaggttccttcattttagttttgatgtgtattttctcttcctctgaaaagagaaagaagccagtcttcatcttcaacttcgctggtaacttggttatgactttgagagctattttcgaagttatcgttttggcttctaacaactactctatcgctgttcaatacggtttcgcttttgctgccgtcagacaatacgttcacgccttcaacattatcatcttgttgttgggtccattcatcttgttcatcgctgaaatgtctttgatgttgcaagttagaatcatttgttcccaacacagaccaactatgattaccaccactgttatctcttgtattttcactgttgttaccttggccttctggatcaccgacatgtctcaagaaattgcttaccaattgttcttgaaaaactacaacatgaagcaaattgttggttactcctggttgtactttatcgctaagatcaccttcgctgcttccattatcttccattcctccgtcttctccttcaaattgatgcgtgctatttacattcgtagaaagatcggtcaattcccattcggtccaatgcaatgtatcttcattgtttcctgtcaatgtttgatcgttccagctattttcactttgatcgattctttcacccacacttacgatggtttctcctccatgactcaatgtttgttgatcatctccttaccattgtcttccttgtgggccacccacaccgctcaaaagttgcaaaccatgaaggataacactaacccaccatctggtacccaattaaccatcagagttgatcgtactttcgacatgaagttcgtttccgactcctctgacggttctttcactgaaaagaccgaagaaactttgcca

SEQ ID NO: 200 - DNA, 1278 bp, Artificial Sequence
  Description of Artificial Sequence Synthetic polynucleotide
atgtccggtaagcaagacttgtctccattaggtttgtactcttcttacgaccctaccaagggtttgatttcttacacctccttgtacggttctggtactactgttactttcgaagaattgcaaatctttgttaacaagaaaattacccaaggtattttgttcggtactagaatcggtgccgccggtttagctatcatcgtcttatggatggtctctaagaacagaaagactccaattttcattattaaccaaatctccttgttcttgatcttgttgcactcctctttgttcttgagatacttgttgggtgattacgcttctgtcgtcttcaactttaccttattctcccaatccatctccagaaacgatgtccacgtctacggtgccaccaacatgattcaagtcttgttggttgccgctgttgaaatttctttgatttttcaagtcagagttattttcaaaggtgattcttacaaaggtgtcggtagaatcttgacctctatctctgccgtcttgggtttcactaccgtcgtcatgtacttcattactgccgttaagtccatgacctccgtttactctgatttgactaagacttccgaccgttacttctttaatatcgcttctattttattgtcttcttccgttaactttatgaccttgttattgaccgtcaagttaattttggccgtcagatctcgtagattcttgggtttgaagcaattcgattccttccatgttttgttgattatgtccttccaaactttgatcttcccatctatcttattcatcttggcttacgccttaaacccaaaccaaggtaccgacactttaacttccattgctaccttgttagtcactttgtctttgcctttgtcttctatgtgggctacctctgctaacaactcctcccacccatcctctatcaacacccaattccgtcaaagaaactatgacgacgtctccttcaagaccggtattacctctttctactccgaatcttctaagccttcttccaagtacagacatactaacaacttatatgacttatacccagtctcccgtacctctaactccagatgtaacggttacccaaacgacggttctaaattagctccaaatccaaactgtgttggtcacaacggttctactatgtccgttaacgacaagaacggtgctcatgctacctgtgttcaaaataacgtcaccttgaacaccgactccactttgaactactctaacgttgacacccaagacacttccaagatcttgatgaccacc

SEQ ID NO: 201 - DNA, 1110 bp, Artificial Sequence
  Description of Artificial Sequence Synthetic polynucleotide
atggcttctatggttccaccaccagattttgacccttacacccaagagttcatggttttaggtccagatggtcaagaaatcccaatctccatgcaaaccgtcaacgaataccgtttgtacaccgctcgtttgggtttggcttatggttcccaaattggtgccaccttattgttattgttggttttgtctttgttaactagaagagaaaagagaaagtccggtatttttattgttaacgctttgtgtttggttactaacaccatcagatgtattttgttgtcctgctttgtcacttccaccttgtggcacccatacacccaattctctcaagatacttccagagtttccaaaactgacgttaacacctctatcgctgcctctattttcactttgattgtcactgttttaatcatgatctccttatctgttcaagtttgggttgtttgtattaccactgctccataccaaagatacatgattatgggtgctaccaccgctactgccatggtcgccgttggttacaaggctgcttttgttatcacttccatcattcaaactttaaacggtcaagacggtggttcctacttggatttggtcatgcaatcttacatcactcaagctgtcgctatttctttctattcctgtattttcacttacaagttaggtcacgctattgttcaaagaagaaccttgaatatgccacaatttggtccaatgcaaattatcttcatcatgggttctttattcactggtttacaattcgtcaagaacgtcgatgaattgggtattatcacccctaccattgtttgtatctttttgccattgtccgctatctgggctggtgtcgtcaacgaaaaggttgtcggtgctaatggtccagacgctcatcacagattgttgcaaggtgaattctacagagctgcttctaactccacttacggttctaactcttccggtactgttgtcgacagatccagacaaatgtctgtctgtacttgtgcttcttcttccccatttgttagaaagaagtctgttgccgaatgggacgatgaagctattttagttggtagagaattcggtttctcccgtggtgaagtcggtgaaagaggt

SEQ ID NO: 202 - DNA, 1044 bp, Artificial Sequence
  Description of Artificial Sequence Synthetic polynucleotide
atgcgtgaaccatggtggaagaactactacaccatgaacggtacccaagtccaaaaccaatccatcccaattttgtccacccaaggttacattcaagttccattgtccaccatcgataaggctgaaagaaacagaattttgactggtatgaccgtttctgctcaattggccttgggtgtcttgatcatggtcatgtctattttgttgtcctccccagaaaagagaaagaccccagttttcatcgtcaactctgcctctatcatttccatgtgtattagagctatcttgatgattgtcaacttgtgttctgaatcctactctttggctgttatgtacggtttcgtcttcgaattggttggtcaatacgttcacgtttttgacattttggttatgattattggtaccatcatcattattaccgctgaagtttccatgttgttgcaagtcagaattatttgtgctcacgacagaaagactcaaagaattgttacctgtatctcttctggtttatccttgatcgtcgttgccttctggttcactgatatgtgtcaagaaattaagtacttgttgtggttgaccccatacaacaaccaccaaatctctggttactactgggtttacttcgtcggtaagatcttgttcgccgtttccattatgttccactctgccgtcttctcctacaagttgttccacgctatccaaattagaaagaagattggtcaattcccattcggtccaatgcaatgtattttaattatttcctgtcaatgtttgttcgttccagctattttcactatcatcgactctttcatccacacttacgacggtttttcctccatgacccaatgtttgttgatcgtctctttgccattgtcctccttgtgggcctcttccactgctttaaagttgcaatctttgaagtctaccacctctccaggtgacactactcaagtttccattagagtcgacagaacctacgacatcaagagaatcccaactgaagaattgtcttctgttgacgaaaccgaaatcaagaagtggcca

SEQ ID NO: 203 - DNA, 1044 bp, Artificial Sequence
  Description of Artificial Sequence Synthetic polynucleotide
atgagacaaccatggtggaaagactttactattcccgatgcatccgcaattattcaccaaaatattaccattgtctctattgtaggagagattgaagtgccagtttcaacaattgatgcatatgaaagagatagacttttaactggaatgactttgtctgcccaacttgctttaggagtccttaccattttgatggtttgtctattgtcatcatccgaaaaacgaaaacacccagtttttgtttttaattcggcaagtattgttgcaatgtgtcttcgggccattttgaatatagtgaccatatgcagcaatagctacagtatcctggttaattacgggtttatcttaaacatggttcatatgtatgtccatgtgtttaatattttaattttgttgcttgcaccggtcatcatttttactgctgagatgagcatgatgattcaagttcgtataatttgtgcacatgatagaaagacacaaaggataatgactgttattagtgcctgcttaactgttttggttctcgcattttggattactaacatgtgtcaacagattcagtatctgttatggttaactccacttagcagcaagaccattgttggatactcttggccctactttattgctaaaatactttttgcttttagcattatttttcacagtggtgttttttcatacaaactctttcgtgccatattaatacggaaaaaaattgggcaatttccatttggtccgatgcagtgtattttagttattagctgccaatgtcttattgttccagctacctttactataatagatagttttatccatacgtatgatggctttagctctatgactcaatgtctgctaatcatttctcttcctctttcgagtttatgggcgtctagtacagctctgaaattgcaaagcatgaaaacttcatctgcgcaaggagaaaccaccgaggtttcgattagagttgatagaacgtttgatatcaaacatactcccagtgacgattattcgatttctgatgaatctgaaactaaaaagtggacg

SEQ ID NO: 204 - DNA, 963 bp, Artificial Sequence
  Description of Artificial Sequence Synthetic polynucleotide
atggatactagtatcaatactctcaaccctgcgaatatcattgtcaactacaccttgccaaatgatcctagagtaattagtgtcccatttggagcttttgacgaatatgttaaccaatctatgcaaaaggccattatccatggagtttccattggttcatgcaccataatgcttttaattattttgatcttcaatgtcaaacgcaagaagtcgccagctttctatcttaattcggttacgttgactgcaatgattattcggtctgctcttaatttggcatatttgctaggtcctttggctggattaagttttacgttctccggcttggtaactccagaaaccaatttctctgtctctgaagccaccaatgctttccaggttattgttgttgctcttatcgaggcgtccatgacatttcaggtgttcgtcgtcttccaatcaccagaagtgaagaagttgggtatagctcttacctccatatctgcattcacgggtgctgctgctgtaggatttactatcaatagtacaatccaacaatcgagaatttatcattcagttgtcaatggaactcctacgccaacggtcgctacctggtcttgggttagagatgtgcctacgatacttttttctacttcggttaacataatgtctttcatcttgattctcaagttagggtttgccataaagacaagaagataccttggccttcggcaatttggcagtttgcacatcttattgatgatggctactcaaacattattggccccatctattctcattcttgtacattacggatatggcacatctctgaatagccagctcattcttataagttacttgcttgttgttttgtctttaccagtatcctctatctgggcagcaacagccaacaattctcctcaacttccatcttccgcaactctttcattcatgaacaaaacgacctctcacttttctgaaagc

SEQ ID NO: 205 - DNA, 1413 bp, Artificial Sequence
  Description of Artificial Sequence Synthetic polynucleotide
atgtctgactccgcccaaaacttgtccgatttggccttcaactcttcttataacccattggactcctttattacctttacctctatctacggtgataacactgctgttaagttctccgttttacaagacatggttgacgttaatactaatgaagccatcgtttacggtacccgttgtggtgcttctgtcttgacccaaattatcatgtggatgatttctaaaaacagaagaaccccagtctttattattaaccaagtttctttgactttgattttaattcactctgccttgtacttcaagtacttgttgtctggtttcggttccgttgtctacggtttgactgctttcccacaattgattaagccaggtgatttgagagctttcgctgctgctaacatcgttatggtcttgttggtcgcttctattgaagcttccttaatcttccaagtcaaagttatcttcaccggtgataacatgaagagagtcggtttaatcttgactattatttgtacttgtatgggtttagctactgttaccatgtactttattactgccgtcaagtctattgtctctttgtaccgtgacatgtctggttcctccaccgttttatataacgtttctttaattatgttggcttcctccatccactttatggctttgatcttggttgtcaaattgttcttggctgttagatctagaagattcttgggtttgaaacaattcgattctttccacattttgttgatcatctcttgtcaaactttgttggttccatctttattattcattattgcttactcttttccatcttctaagaacattgaatctttgaaggctatcgctgttttgaccgtcgttttgtctttgccattgtcttctatgtgggctactgctgctaataacttcactaactcttcctcctccggttccgactccgctccaaccaatggtggtttctacggtagaggttcttccaacttgtatcctgaaaagactgataacagatccccaaagggtgccagaaacgctttatacgaattaagatctaagaacaatgctgagggtcaagctgatatttacaccgttaccgatattgaaaacgatattttcaacgatttgtccaagccagttgagcaaaacattttctctgatgttcaaattattgattctcattctttgcataaggcttgttctaaagaagacccagtcatgactttgtacactccaaacactgctattgaaggtgaggagagaaaattgtggacttctgactgttcctgttccactaacggttccaccccagttaagaagaagtccaccggtgaatacgccaatttaccaccacacttattaagatatgatgaaaactacgatgaagaagctggtggtagacgtaaggcctccttgaaatgg

SEQ ID NO: 206 - DNA, 1101 bp, Artificial Sequence
  Description of Artificial Sequence Synthetic polynucleotide
atggagcaaatcccagtctacgagcgtccaggtttcaacccacacaagcaaaacattaccttgttcaagcatgatggttctactgttactgtcggtttgcatgagttggacgccatgttcactcattccatcagagttgctgtcgtcttcgcctctcaaattggtgcttgtgctttgttgtctgttatcgttgctatggtcaccaagagagaaaagagacgtgctttgttcttcttgcacattatttccttgttgttggtcgttgttcgttccgtcttgcaaatcttgtacttcgtcggtccatgggctgaaacttataattacgtcgcctactactatgaagacattcctttgtctgacaaattgatttccatttgggctggtattatccaattgattttgaatatctgtattttgttatctttgatcttgcaagttcgtgtcgtttacgccacctctccaaaattgaacactattatgactttagtctcttgtgttatcgcttctatttctgtcggtttcttctttactgtcatcgttcaaatttctgaggctattttaaacggtgttggttacgacggttgggtttacaaagtccatagaggtgtcttcgctggtgctatcgccttcttctctttcatcttcatctttaagttggccttcgctatcagaagaagaaaggctttgggtttgcaaagattcggtccattgcaagttatcttcatcatgggttgtcaaactatgattgttccagctatctttgctactttggaaaacggtgttggtttcgaaggtatgtcctctttgactgctaccttggctgtcatttccttaccattgtcttctatgtgggccgccgctcaaaccgacggtccatctccacaatccactccaagagacggttatagaagattctctactcgtagatctgccttgaacagatctgacccatctggtggtagatctgttgacatgaacaccttggactctaccggtaacgattccttagctttgcacgttgataagacttttactgttgaatcttccccatcctcccaatctcaagctggtccacacaaggaaagaggtttcgaattcgcc

SEQ ID NO: 207 - DNA, 1152 bp, Artificial Sequence
  Description of Artificial Sequence Synthetic polynucleotide
atgagttcccaatcacacccaccgctaatcgatttattttacgattccagttatgaccctggtgaaagtttaatttattacacatccatctatggtaataatacatacataacttttgatgaactccagacgatagtgaacaagaaggtcacacaaggtatcttatttggtgtcagatgtggtgctgctttcctgatgttggtagcaatgtggttgatttccaaaaataaaagatctagaattttcattaccaaccaatgttgtctggtcttcatgataatgcattctggtctttattttaggtacctgctttcaaggtacggttcagttactttcattctaacagggttccaacaactgcttacaagaaatgacattcatatttatggagctactgattttatccaagtagctttggtagcttgcatagaattatctcttattttccaaataaaagtgatattcgctggtacaaactatggtaagttggctaattatttcatcactctaggttcattattgggtttagccacctttggtatgtacatgcttactgctattaacggtacaataaaattatacaataacgaatatgacccaaaccaaaggaaatactttaacatttctacaatattgcttgcatcatcaattaatatgctaacgctgatacttatattgaagctggtggcagcaattagaacaagacgttacttaggtttgaagcaattcgatagttttcacatcctattaatcatgtcgactcaaacattaataattccttctatcttatttattctatcatacagtttgagagaggatatgcatactgatcaattaataatcatcggaaatctgatcgtggtattgtcattaccattgtcctcaatgtgggcttcgtctctaaacaattcaagtaaacctacatctttgaatactgatttctcagggccaaaatcaagtgaagaagggacagcaataagtttgctatcacaaaacatggaaccatcaatagtcactaaatatacaagaagatcacctgggttatacccagtaagcgtgggtacaccaattgaaaaagaagcatcatacactctttttgaagctactgacattgattttgaaagcagtagtaacgatatcacaaggacttca

SEQ ID NO: 208 - DNA, 1419 bp, Artificial Sequence
  Description of Artificial Sequence Synthetic polynucleotide
atgtcaggaattgatgatatgggtgataaaccagatattttaggtttattttatgatgctaactatgatccaggtcaaggtatactcacatttatttcaatgtacgggaatactactataacttttgatgagttacagttagaggtcaatagtttaattacaagtggtattatgttcggcgtcagatgtggtgctgcttgtttgacattgttaataatgtggatgatttctaagaataagaagactccaatttttattattaatcaatgctcgctaatccttattattatgcattcaggtttatattttaagaatattctatcaaatttgaattctttatcatatatcttaactgggtttactcaaaatatcactaaaaataatatacatgtctttggtgccgctaatattattcaagttttattagtagcaaccattgaactgtcgttagtgtttcaaattcgagtcatgtttaaaggtgacagttttagaaaagctggttacggtttgttgtcaattgcgtctggtttgggtatagctactgtcgtcatgtatttttactctgccattacaaatatgattgctgtttataatcaaacttacaactccactgctaaattatttaacgttgcaaacattcttctgtctacatcgataaattttatgacggtagtattaattgttaaattatttttggctgttagatcaagaagatatttgggtttaaagcagttcgatagtttccatattttattgattatgtcatgtcaaacattgattgtaccatcaattctttttatcttatcatacgctttaagtactaagctgtacactgatcatttagttgtcattgcaactttattagtcgttctatctttaccattatcttcgatgtgggcaagcgctgcaaataattctcctaaaccaagctcgtttacaaccgattattcaaacaagaatcctagtgacacaccaagcttctacagtcaaagtattagttcctcgatgaaaagcaaattcccaagcaaattcatacccttcaatttcaagtctaaagacaattcttctgacactagatcagaaaatacatatattggcaattatgacatggaaaagaatggatcaccaaatcactcttattcttccaaagatcaaagtgaagtttacactataggtgtaagctctatgcacacagatataaagtcacaaaagaatatcagtggacagcatttatataccccaagtacagagattgatgaagaagctagagacttctgggcgggcagagctgttaataattcagttccaaatgactatcaaccatctgagttaccagcatcgattcttgaagaattgaattcactggatgaaaataatgaaggtttcttggagacaaaaagaataacatttagaaaacaa

SEQ ID NO: 209 - DNA, 1107 bp, Artificial Sequence
  Description of Artificial Sequence Synthetic polynucleotide
atgcaattgccaccacgtccagacttcgacattgccactttggttgcctctatcactgttccagaaactgaattggtcttgggtcaaatgccattgggtgctttagaacaattgtaccaaaacagattgcgtttggctattttgttcggtgtcagagtcggtgctgctgttttgaccttgattgctatgcacttaatctccaagaagaacagaaccaagatcttgttcttggctaaccaaatgtctttgatcatgttgatcatccatgctgctttgtacttcagattcttgttgggtccattcgcctccatgttgatgatggttgcttacatcgttgatccaagatctaacgtctctaacgatatctctgtttctgttgccaccaacgttttcatgatgttgatgattatgtccgtccaattgtctttggctgttcaaacccgttctgttttccacgcttggttgaagtctcgtatttacgttaccgttggtttaatcttgttgtccttggtcgtcttcgtcttctggaccacccacactatcgtttcttgtatcgttttaacccatccaactagagacttgccatctatgggttggactagattagcttctgacgtttccttcgcttgttctatctctttcgcttctttggtcttgttggctaagttggtcaccgccatcagagttagaaagaccttgggtaagaagccattgggttacaccaaggttttggtcatcatgtccactcaatctttagtcgttccatctatcttgattatcgttaactacgctttgccagaaaaaaactcttggatcttgtctggtgtcgcttacttgatggttgttttgtccttaccattgtcctccatttgggctaccgccgtccatgacgacgaaatgcaatccaactacttgttgtctgccttgaaagatggtcacgttcaaccatccgaatctaagttgaagactgttttcttgaacagattgagaccattctctactaccactaacagagacgatgaatcctctgttgattccccagccatgccatctccagaatctgatgttaccttcttgaacactggtttcgaatgtgacgaaaagatg

SEQ ID NO: 210 - DNA, 1359 bp, Artificial Sequence
  Description of Artificial Sequence Synthetic polynucleotide
atgtctggtttggctaacaacacctcttacaacccattggaatctttcattattttcacttctgtttacggtggtgataccatggttaagttcgaagacttgcaattagtcttcaccaagcgtattactgaaggtattttgttcggtgtcaaggttggtgccgcttctttgactatgattgttatgtggatgatttccagaagaagaacctccccaatcttcatcatgaaccaattgtctttggttttcaccatcttgcacgcttctttttactttaagtacttattggacggtttcggttctattgtctacactttgaccttgttcccacaattaattacttcctctgacttgcacgttttcgctactgctaacgttgttgaagtcttattggtttcttccatcgaagcctctttggttttccaagtcaacgtcatgttcgctggttctaaccacagaaagttcgcttggttgttggtcggtttctctttgggtttggctttggccactgtcgctttgtacttcgttactgctgtcaagatgatcgcttccgcttacgcttctcaaccaccaactaacccaatctacttcaacgtttccttgttcttgttggctgcctccgttttcttgatgactttaatgttgaccgtcaagttgatcttggctatcagatccagaagattcttgggtttgaagcaattcgactctttccacattttgttgattatgtcttgtcaaactttgatcgctccatctgttttgtacatcttgggttttattttggatcacagaaagggtaacgactacttgattaccgtcgctcaattgttggtcgttttgtctttgccattgtcctccatgtgggccactactgctaacgatgcttcctccggtacttctatgtcttccaaggaatccgtctacggttctgattccttatactctaagtctaagtgttcccaattcaccagaaccttcatgaacagattctctactaagccaactaagaacgacgaaatttctgattccgctttcgtcgctgttgattccttggaaaagaacgctccacaaggtatctctgaacacgtttgtgaattcccacaatctgacttatctgatcaagctacttccatctcctccagaaaaaaggaagctgttgtttacgcttccactgttgatgaagataagggttctttctcctctgacatcaacggttacactgttaccaacatgccattggcttccgctgcttctgctaactgtgaaaactccccatgtcacgttccaagaccatacgaagaaaacgaaggtgtcgtcgaaaccagaaaaattattttgaagaagaacgtcaaatggtag

SEQ ID NO: 211 - DNA, 1332 bp, Artificial Sequence
  Description of Artificial Sequence Synthetic polynucleotide
atgagtgagattaacaattctacctacaatccaatgaatgcatatgtaacgtttacatcaatatatggtgatgatactatggtacgtttcaaagatgtggaattggtagttaacaaaagggttacagaagccattatgttcggcgtcaaagttggtgcagcttcgttgacactcatcatcatgtggatgatctctaagaaaagaacaacaccgatatttatcataaatcagtcttcgcttgtatttaccataatacatgcttcgctttattttgggtaccttttgtcaggatttggtagtatagtttacaatatgacatcgttcccgcagttaataagctccaatgacgttcgtgtgtacgcagctacaaatatttttgaggtcctgttggtagcatctatcgaaatctctctggtttttcaggtcaaagttatgtttgccaacaataatggtcgaagatggacttggtgtttgatggtagtttccatagggatggcactagctactgtaggactttattttgccactgccgttgagttgatcagagctgcttacagcaatgatactgttagccgccatgttttttacaatgtttctctgatcttactagcgtcatctgtcaatctaatgacactaatgctagtggtaaaattagtattagcgatcagatcaagaagatttttggggttaaaacagtttgacagtttccacatattacttataatgtcttgccagactctaatagcaccttccattctattcattttgggttggaccttagaccctcatactggtaatgaggttttaattacagttggtcaattgctaatagtactgtcattaccgctgtcatctatgtgggctacaaccgctaacaataccagttcatctagtagttcggtgtcctgtaatgacagctcttttggtaatgacaatctctgttccaagagttcgcaatttagaagaacttttatgaatagattccgtcccaagtcggttaatggtgacggtaattctgaaaatacctttgttacaattgatgatttggaaaaaagcgtttttcaagaattatcaacacctgttagcggagaatcaaagatagatcatgatcatgcaagtagtatttcatgtcaaaagacatgtaatcatgttcatgcttcgacagtgaattcagataagggatcttggtcctctgatggtagttgtggcagttctccgttaagaaagacttccaccgttaattctgaagatttacctccacatatattgagcgcctacgatgacgatcgaggtatagtagaaagtaaaaaaattatcctaaagaaattatag

SEQ ID NO: 212 - protein, 88 aa, Artificial Sequence
  Description of Artificial Sequence Synthetic polypeptide
RFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 213 - DNA, 252 bp, Artificial Sequence
  Description of Artificial Sequence Synthetic polynucleotide
agatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaaga

SEQ ID NO: 214 - DNA, 264 bp, Artificial Sequence
  Description of Artificial Sequence Synthetic polynucleotide
agatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagaggctgaagct

SEQ ID NO: 215 - DNA, 29 bp, Beauvaria bassiana
ggtgtatgagaccaggtcaaccatgttgg

SEQ ID NO: 216 - DNA, 27 bp, Botrytis cinerea
tggtgtggtagaccaggtcaaccatgt

SEQ ID NO: 217 - DNA, 39 bp, Candida albicans
ggtttcagattgaccaacttcggttacttcgaaccaggt

SEQ ID NO: 218 - DNA, 48 bp, Candida guilliermondii
aagaagaactctagattcttgacctactggttcttccaaccaatcatg

SEQ ID NO: 219 - DNA, 45 bp, Candida lusitaniae
aagtggaagtggatcaagttcagaaacaccgacgttatcggttag

SEQ ID NO: 220 - DNA, 51 bp, Geotrichum candidum
ggtgactggggttggttctggtacgttccaagaccaggtgacccagctatg

SEQ ID NO: 221 - DNA, 30 bp, Hypocrea jecorina
tggtgttacagaatcggtgaaccatgttgg

SEQ ID NO: 222 - DNA, 37 bp, Komagataella pastoris
cagatggagaaacaacgaaaagaaccaaccattcggt

SEQ ID NO: 223 - DNA, 35 bp, Lodderomyces elongisporous
ggatgtggaccagatacggtagattctctccagtt

SEQ ID NO: 224 - DNA, 26 bp, Paracoccidioides brasiliensis
ggtgtaccagaccaggtcaaggttgt

SEQ ID NO: 225 - DNA, 30 bp, Pseudogymnoascus destructans
ttctgttggagaccaggtcaaccatgtggt

SEQ ID NO: 226 - DNA, 39 bp, Saccharomyces cerevisiae
tggcactggttgcaattgaagccaggtcaaccaatgtac

SEQ ID NO: 227 - DNA, 69 bp, Schizosaccharomyces japonicus
gtttctgacagagttaagcaaatgttgtctcactggtggaacttcagaaacccagacaccgctaacttg

SEQ ID NO: 228 - DNA, 69 bp, Schizosaccharomyces octosporus
acctacgaagacttcttgagagtttacaagaactggtggtctttccaaaacccagacagaccagacttg

SEQ ID NO: 229 - DNA, 39 bp, Vanderwaltozyma polyspora
tggcactggttggaattggacaacggtcaaccaatctac

SEQ ID NO: 230 - DNA, 36 bp, Zygosaccharomyces rouxii
cacttcatcgaattggacccaggtcaaccaatgttc

SEQ ID NO: 231 - DNA, 21 bp, Artificial Sequence
  Description of Artificial Sequence Synthetic oligonucleotide
cagaatcaaaaatgtctgatg

SEQ ID NO: 232 - DNA, 19 bp, Artificial Sequence
  Description of Artificial Sequence Synthetic oligonucleotide
atgaggaagccagaaagtt

SEQ ID NO: 233 - DNA, 20 bp, Artificial Sequence
  Description of Artificial Sequence Synthetic oligonucleotide
catacaagtcagcaataata

SEQ ID NO: 234 - DNA, 19 bp, Artificial Sequence
  Description of Artificial Sequence Synthetic oligonucleotide
atagttcagaaaatactgc

SEQ ID NO: 235 - DNA, 20 bp, Artificial Sequence
  Description of Artificial Sequence Synthetic oligonucleotide
aaaactgcagtaaaaattga

SEQ ID NO: 236 - DNA, 19 bp, Artificial Sequence
  Description of Artificial Sequence Synthetic oligonucleotide
attggttgcagttaaaacc

SEQ ID NO: 237 - DNA, 20 bp, Artificial Sequence
  Description of Artificial Sequence Synthetic oligonucleotide
cgctaaaataaaagtgagaa

SEQ ID NO: 238 - DNA, 19 bp, Artificial Sequence
  Description of Artificial Sequence Synthetic oligonucleotide
actggttgcaactcaagcc

SEQ ID NO: 239 - DNA, 20 bp, Artificial Sequence
  Description of Artificial Sequence Synthetic oligonucleotide
aaagaccagcagtgaaaaga

SEQ ID NO: 240 - DNA, 20 bp, Artificial Sequence
  Description of Artificial Sequence Synthetic oligonucleotide
ttccacacaagccactcaga

SEQ ID NO: 241 - DNA, 20 bp, Artificial Sequence
  Description of Artificial Sequence Synthetic oligonucleotide
aaaatacacactccaccaag

SEQ ID NO: 242 - DNA, 20 bp, Artificial Sequence
  Description of Artificial Sequence Synthetic oligonucleotide
gcaaagaattcatcagaccc

SEQ ID NO: 243 - DNA, 20 bp, Artificial Sequence
  Description of Artificial Sequence Synthetic oligonucleotide
tctttgtttgaaacttattt

SEQ ID NO: 244 - DNA, 20 bp, Artificial Sequence
  Description of Artificial Sequence Synthetic oligonucleotide
ttgtacatgaaactaaatat

SEQ ID NO: 245 - DNA, 20 bp, Artificial Sequence
  Description of Artificial Sequence Synthetic oligonucleotide
gtaagatggtggataaaaat

SEQ ID NO: 246 - DNA, 20 bp, Artificial Sequence
  Description of Artificial Sequence Synthetic oligonucleotide
catctttgtatacgtctgac

SEQ ID NO: 247 - DNA, 20 bp, Artificial Sequence
  Description of Artificial Sequence Synthetic oligonucleotide
aataaccaatagtagaacag

SEQ ID NO: 248 - DNA, 20 bp, Artificial Sequence
  Description of Artificial Sequence Synthetic oligonucleotide
ctgttctactattggttatt

SEQ ID NO: 249 - DNA, 20 bp, Artificial Sequence
  Description of Artificial Sequence Synthetic oligonucleotide
atattcaagatttttttctg

SEQ ID NO: 250 - DNA, 20 bp, Artificial Sequence
  Description of Artificial Sequence Synthetic oligonucleotide
atgtgtaaatgaaggaataa

SEQ ID NO: 251 - DNA, 20 bp, Artificial Sequence
  Description of Artificial Sequence Synthetic oligonucleotide
tgaagtcagtaaagctactc

SEQ ID NO: 252 - DNA, 20 bp, Artificial Sequence
  Description of Artificial Sequence Synthetic oligonucleotide
tcctcgtgggccaggactag

SEQ ID NO: 253 - DNA, 20 bp, Artificial Sequence
  Description of Artificial Sequence Synthetic oligonucleotide
cattctacctctagggaagc

SEQ ID NO: 254 - protein, 14 aa, Alternaria brasicicola
WSFTQKRPYGLPIG

SEQ ID NO: 255 - protein, 8 aa, Arthrobotrys oligospora
WCPYNSCP

SEQ ID NO: 256 - protein, 12 aa, Ashbya aceri
WHWLRFGDGQSM

SEQ ID NO: 257 - protein, 13 aa, Aspergillus clavatus
QWCELPGQGCYMI

SEQ ID NO: 258 - protein, 12 aa, Aspergillus flavus
WCSLPAQGCYML

SEQ ID NO: 259 - protein, 12 aa, Aspergillus fumigata
WCHLPGQGCYML

SEQ ID NO: 260 - protein, 12 aa, Aspergillus kawachii
WCHLPGQPCNMI

SEQ ID NO: 261 - protein, 12 aa, Aspergillus nidulans
WCRFAGRICPPT

SEQ ID NO: 262 - protein, 12 aa, Aspergillus niger
WCVLPGQPCNMI

SEQ ID NO: 263 - protein, 10 aa, Aspergillus ruber
WCALPGQICS

SEQ ID NO: 264 - protein, 12 aa, Aspergillus terreus
WCWLPGQGCYML

SEQ ID NO: 265 - protein, 10 aa, Beauveria bassiana
WCMRPGQPCW

SEQ ID NO: 266 - protein, 10 aa, Botryosphaeria parva
WCRWKGQPCS

SEQ ID NO: 267 - protein, 13 aa, Candida dubliniensis
KFKLTNFGYFEPG

SEQ ID NO: 268 - protein, 16 aa, Candida guilliermondii
KKNSRFLTYWFFQPIM

SEQ ID NO: 269 - protein, 13 aa, Candida lusitaniae
WKWIKFRNTDVIG

SEQ ID NO: 270 - protein, 13 aa, Capronia coronata
LSYWKGVNDGGSS

SEQ ID NO: 271 - protein, 13 aa, Capronia epimyces
LSYWAGVNDGGSS

SEQ ID NO: 272 - protein, 11 aa, Chaetomium globosum
WCKQFLGMPCW

SEQ ID NO: 273 - protein, 12 aa, Chaetomium thermophilum
SWCTRFPGQPCW

SEQ ID NO: 274 - protein, 10 aa, Chryphonectria parasitica
WCLFHGEGCW

SEQ ID NO: 275 - protein, 10 aa, Claviceps purpurea
WCWRPGQGCW

SEQ ID NO: 276 - protein, 9 aa, Coccidioides immitis
WCQRPGEPC

SEQ ID NO: 277 - protein, 10 aa, Colletotrichum gloeosporioides
WCTKPGQPCW

SEQ ID NO: 278 - protein, 14 aa, Coniosporium apollinis
WGSRFCHKTGQGCP

SEQ ID NO: 279 - protein, 14 aa, Debaryomyces hansenii
KFHWMTYRFFQPNL

SEQ ID NO: 280 - protein, 14 aa, Endocarpon pusillum
WWGFRWSRHGTSSW

SEQ ID NO: 281 - protein, 13 aa, Eremothecium cymbalariae
WHWLRFDRGQPIH

SEQ ID NO: 282 - protein, 10 aa, Fusarium oxysporum
WCTWRGQPCW

SEQ ID NO: 283 - protein, 10 aa, Fusarium pseudograminearum
WCTWKGQPCW

SEQ ID NO: 284 - protein, 12 aa, Gaeumannomyces graminis
QNGCQYRGQSCW

SEQ ID NO: 285 - protein, 16 aa, Geotrichum candidum
DWGWFWYVPRPGDPAM

SEQ ID NO: 286 - protein, 10 aa, Gibberella fujikuroi
WCTWRGQPCW

SEQ ID NO: 287 - protein, 10 aa, Gibberella moniliformis
WCTWRGQPCW

SEQ ID NO: 288 - protein, 10 aa, Gibberella zeae
WCWWKGQPCW

SEQ ID NO: 289 - protein, 10 aa, Glarea lozoyensis
QCIRHGQPCW

SEQ ID NO: 290 - protein, 11 aa, Grosmannia clavigera
QWCQWYGQACW

SEQ ID NO: 291 - protein, 14 aa, Kazachstania africana
WHWLSIAPGQPMYI

SEQ ID NO: 292 - protein, 13 aa, Kazachstania naganishii
WHWLRLSYGQPIY

SEQ ID NO: 293 - protein, 13 aa, Kluyveromyces marxianus
WKWLSLRVGQPIY

SEQ ID NO: 294 - protein, 13 aa, Kluyveromyces waltii
WRWLSLARGQPMY

SEQ ID NO: 295 - protein, 14 aa, Kuraishia capsulata
RLGARIYAKGQPIY

SEQ ID NO: 296 - protein, 13 aa, Lachancea kluyveri
WHWLSFSKGEPMY

SEQ ID NO: 297 - protein, 13 aa, Lachancea thermotolerans
WRWLSLSRGQPMY

SEQ ID NO: 298 - protein, 12 aa, Lodderomyces elongisporus
WMWTRYGRFSPV

SEQ ID NO: 299 - protein, 11 aa, Magnaporthe oryzae
QWCPRRGQPCW

SEQ ID NO: 300 - protein, 12 aa, Magnaporthe poae
QNGCPYPGQSCW

SEQ ID NO: 301 - protein, 9 aa, Marssonina brunnea
CGYRGQPCP

SEQ ID NO: 302 - protein, 10 aa, Metarhizium acridum
WCWQPGQPCW

SEQ ID NO: 303 - protein, 10 aa, Metarhizium anisopliae
WCWRPGQPCW

SEQ ID NO: 304 - protein, 14 aa, Mycosphaerella pini
GVLTRCTVPGLACG

SEQ ID NO: 305 - protein, 10 aa, Nectria haematococca
WCFYPGQPCW

SEQ ID NO: 306 - protein, 12 aa, Neosartorya fischeri
WCHLPGQGCYML

SEQ ID NO: 307 - protein, 11 aa, Neurospora tetrasperma
QWCRIHGQSCW

SEQ ID NO: 308 - protein, 13 aa, Ogataea parapolymorpha
WGWHRVNRNEVIF

SEQ ID NO: 309 - protein, 11 aa, Ophiostoma piceae
QWCPMVGQPCW

SEQ ID NO: 310 - protein, 9 aa, Paracoccidioides lutzii
WCTRPGQGC

SEQ ID NO: 311 - protein, 10 aa, Penicillium chrysogenum
WCGHIGQGCY

SEQ ID NO: 312 - protein, 10 aa, Penicillium digitatum
WCGHIGQGCY

SEQ ID NO: 313 - protein, 10 aa, Penicillium oxalicum
WCAHPGQGCA

SEQ ID NO: 314 - protein, 10 aa, Penicillium roqueforti
WCGHIGQGCY

SEQ ID NO: 315 - protein, 14 aa, Phaeosphaeria nodorum
YNGWRYRPYGLPVG

SEQ ID NO: 316 - protein, 13 aa, Pichia sorbitophila
FHWFKYNKYDPIT

SEQ ID NO: 317 - protein, 12 aa, Podospora anserina
QWCLRFVGQSCW

SEQ ID NO: 318 - protein, 14 aa, Pyrenophora teres f teres
VTWTQKRPYGMPVG

SEQ ID NO: 319 - protein, 13 aa, Pyrenophora tritici-repentis
SWTQKRPYGMPVG

SEQ ID NO: 320 - protein, 13 aa, Saccharomyces bayanus
WHWLQLKPGQPMY

SEQ ID NO: 321 - protein, 13 aa, Saccharomyces dairenensis
WHWLRLDPGQPLY

SEQ ID NO: 322 - protein, 13 aa, Saccharomyces mikatae
WHWLQLKPGQPMY

SEQ ID NO: 323 - protein, 13 aa, Saccharomyces paradoxis
WHWLQLKPGQPMY

SEQ ID NO: 324 - protein, 24 aa, Schizosaccharomyces octosporus
KTYEDFLRVYKNWWSFQNPDRPDL

SEQ ID NO: 325 - protein, 9 aa, Sclerotinia borealis
WCGRPGQPC

SEQ ID NO: 326 - protein, 9 aa, Sclerotinia sclerotiorum
WCGRPGQPC

SEQ ID NO: 327 - protein, 11 aa, Sordaria macrospora
QWCRIHGQSCW

SEQ ID NO: 328 - protein, 10 aa, Sporothrix schenckii
YCPLKGQSCW

SEQ ID NO: 329 - protein, 12 aa, Tetrapisispora blattae
HWLRLGRGEPLY

SEQ ID NO: 330 - protein, 13 aa, Tetrapisispora phaffii
WHWLRLDPGQPLY

SEQ ID NO: 331 - protein, 11 aa, Thielavia heterothallica
WCVQFLGMPCW

SEQ ID NO: 332 - protein, 10 aa, Togninia minima
WCTKHGQSCW

SEQ ID NO: 333 - protein, 10 aa, Trichoderma atroviridis
WCWRVGESCW

SEQ ID NO: 334 - protein, 10 aa, Trichoderma jecorina
WCYRIGEPCW

SEQ ID NO: 335 - protein, 11 aa, Trichoderma virens
WCYRVGMTCGW

SEQ ID NO: 336 - protein, 10 aa, Verticillium alfalfae
PCPRPGQGCW

SEQ ID NO: 337 - protein, 10 aa, Verticillium dahliae
PCPRPGQGCW

SEQ ID NO: 338 - protein, 13 aa, Wickerhamomyces ciferrii
WQWRKYLNGSPNY

SEQ ID NO: 339 - protein, 12 aa, Capronia coronata
SYWKGVNDGGSS

SEQ ID NO: 340 - protein, 10 aa, Dactylellina haptotyla
WCVYNSCPKT

SEQ ID NO: 341 - protein, 12 aa, Phaeosphaeria nodorum
GWRYRPYGLPVG

SEQ ID NO: 342 - DNA, 1041 bp, Artificial Sequence
  Description of Artificial Sequence Synthetic polynucleotide
atggcctcaaacggctggcaaaacaatgcaacatttgatccatatgctcagacgttcgtgttactacagccagatggtctaactccattcccagcgttgctaggtgatgttttagctttgaatactgtcagcgttacccaaggtattatttatggcacacaagtcggtatctccggcttgcttttactgatactattgattatgactaaaccagacaagagaagaagtttggtgttcatcctgaatagtctttctctactgttgatctttgccagaaacgtgttgagttgtgtgcaattgactactatattttataacttttataactgggagttgcactggtaccctgaaagccctgcattatcaagagctatggatctatctgccgcaactgaagtgttaaatataccaatagacgtggccatcttctcatccttggtagttcaagttcatatagtttgttgcacgatacatacactggtgaggacctcagcactgttatctagtgccgcggttggtctggccgctgtggctgttagatttgctctggctgtggttaatatcaaatacagtatttttggtattaatacattgactgaaccccaatttaacttaatagtacaccttaaaagggtaagtgatatactgacagtggttgctatcgcatttttctctagcattttcgtcgctaagttgggagtggcgattcacactagaagaacgctaaatttaaagaatttcggtgctattcaaatcatattcataatgggatgtcaaactatgttgattcctttaatatttgttatagtgtctttctatgcttctagaggatctcaaattgggagcatggttcctacagtggttgcaacctttttgcccctatcaggtatgtgggctagcgctcaaacgaataacgaaaaaatggggagggctgaccaacgtttccatcgtgcagtccctgtgggcgcgactgatttctcagtgactaaggctagaagcgcaaaagccagtgacactctagatacactaatcggtgacgac

SEQ ID NO: 343 - protein, 13 aa, Artificial Sequence
  Description of Artificial Sequence Synthetic peptide
GFRLTNFGYFEPG

SEQ ID NO: 344 - protein, 13 aa, Artificial Sequence
  Description of Artificial Sequence Synthetic peptide
AFRLTNFGYFEPG

SEQ ID NO: 345 - protein, 13 aa, Artificial Sequence
  Description of Artificial Sequence Synthetic peptide
GFALTNFGYFEPG

SEQ ID NO: 346 - protein, 13 aa, Artificial Sequence
  Description of Artificial Sequence Synthetic peptide
GFRLTNAGYFEPG

SEQ ID NO: 347 - protein, 13 aa, Artificial Sequence
  Description of Artificial Sequence Synthetic peptide
GFRLTNFGAFEPG

SEQ ID NO: 348 - protein, 13 aa, Artificial Sequence
  Description of Artificial Sequence Synthetic peptide
GFRLTNFGYAEPG

SEQ ID NO: 349 - protein, 13 aa, Artificial Sequence
  Description of Artificial Sequence Synthetic peptide
GFRLTNFGYFEAG

SEQ ID NO: 350 - protein, 13 aa, Artificial Sequence
  Description of Artificial Sequence Synthetic peptide
GFRLTNFGYFEPA

SEQ ID NO: 351 - protein, 19 aa, Saccharomyces cerevisiae
KREAEAWHWLQLKPGQPMY

SEQ ID NO: 352 - DNA, 60 bp, Artificial Sequence
  Description of Artificial Sequence Synthetic primer
gatatttatatgctataaagaaattgtactccagatttcccatatatgacccttctagac

SEQ ID NO: 353 - DNA, 60 bp, Artificial Sequence
  Description of Artificial Sequence Synthetic primer
tcataccaaaataaaaagagtgtctagaagggtcatatatgggaaatctggagtacaatt

SEQ ID NO: 354 - DNA, 20 bp, Artificial Sequence
  Description of Artificial Sequence Synthetic primer
ggctgcactcattccggtac

SEQ ID NO: 355 - DNA, 24 bp, Artificial Sequence
  Description of Artificial Sequence Synthetic primer
acggacgtttaggatgacgtattg

SEQ ID NO: 356 - DNA, 60 bp, Artificial Sequence
  Description of Artificial Sequence Synthetic primer
gctatttctagctctaaaacatatttagtttcatgtacaactgccaatcgcagctcccag

SEQ ID NO: 357 - DNA, 60 bp, Artificial Sequence
  Description of Artificial Sequence Synthetic primer
ctgggagctgcgattggcagttgtacatgaaactaaatatgttttagagctagaaatagc

SEQ ID NO: 358 - DNA, 60 bp, Artificial Sequence
  Description of Artificial Sequence Synthetic primer
gctatttctagctctaaaacaaataagtttcaaacaaagagatcatttatctttcactgc

SEQ ID NO: 359 - DNA, 60 bp, Artificial Sequence
  Description of Artificial Sequence Synthetic primer
gcagtgaaagataaatgatctctttgtttgaaacttatttgttttagagctagaaatagc

SEQ ID NO: 360 - DNA, 60 bp, Artificial Sequence
  Description of Artificial Sequence Synthetic primer
agcaaaagcctcgaaatacgggcctcgattcccgaactaccataatagattgccttctta

SEQ ID NO: 361 - DNA, 60 bp, Artificial Sequence
  Description of Artificial Sequence Synthetic primer
ccactggaaagcttcgtgggcgtaagaaggcaatctattatggtagttcgggaatcgagg

SEQ ID NO: 362 - DNA, 20 bp, Artificial Sequence
  Description of Artificial Sequence Synthetic primer
gttaggcgggcaagagagac

SEQ ID NO: 363 - DNA, 24 bp, Artificial Sequence
  Description of Artificial Sequence Synthetic primer
cggaacaaattagccacatcgacg

SEQ ID NO: 364 - DNA, 60 bp, Artificial Sequence
  Description of Artificial Sequence Synthetic primer
gctatttctagctctaaaacgggtctgatgaattctttgcctgccaatcgcagctcccag

SEQ ID NO: 365 - DNA, 60 bp, Artificial Sequence
  Description of Artificial Sequence Synthetic primer
ctgggagctgcgattggcaggcaaagaattcatcagacccgttttagagctagaaatagc

SEQ ID NO: 366 - DNA, 60 bp, Artificial Sequence
  Description of Artificial Sequence Synthetic primer
gctatttctagctctaaaaccttggtggagtgtgtattttgatcatttatctttcactgc

SEQ ID NO: 367 - DNA, 60 bp, Artificial Sequence
  Description of Artificial Sequence Synthetic primer
gcagtgaaagataaatgatcaaaatacacactccaccaaggttttagagctagaaatagc

SEQ ID NO: 368 - DNA, 70 bp, Artificial Sequence
  Description of Artificial Sequence Synthetic primer
aaaaggggcctgtctcactaccaacatggttgacctggtctcatacaccaagcttcagcctctcttttat

SEQ ID NO: 369 - DNA, 70 bp, Artificial Sequence
  Description of Artificial Sequence Synthetic primer
ataaaagagaggctgaagcttggtgtatgagaccaggtcaaccatgttggtagtgagacaggcccctttt

SEQ ID NO: 370 - DNA, 67 bp, Artificial Sequence
  Description of Artificial Sequence Synthetic primer
aaaaggggcctgtctcactaacatggttgacctggtctaccacaccaagcttcagcctctcttttat

SEQ ID NO: 371 - DNA, 67 bp, Artificial Sequence
  Description of Artificial Sequence Synthetic primer
ataaaagagaggctgaagcttggtgtggtagaccaggtcaaccatgttagtgagacaggcccctttt

SEQ ID NO: 372 - DNA, 79 bp, Artificial Sequence
  Description of Artificial Sequence Synthetic primer
aaaaggggcctgtctcactaacctggttcgaagtaaccgaagttggtcaatctgaaaccagcttcagcctctcttttat

SEQ ID NO: 373 - DNA, 79 bp, Artificial Sequence
  Description of Artificial Sequence Synthetic primer
ataaaagagaggctgaagctggtttcagattgaccaacttcggttacttcgaaccaggttagtgagacaggcccctttt

SEQ ID NO: 374 - DNA, 82 bp, Artificial Sequence
  Description of Artificial Sequence Synthetic primer
aaaaggggcctgtctcactaaccgataacgtcggtgtttctgaacttgatccacttccacttagcttcagcctctcttttat

SEQ ID NO: 375 - DNA, 82 bp, Artificial Sequence
  Description of Artificial Sequence Synthetic primer
ataaaagagaggctgaagctaagtggaagtggatcaagttcagaaacaccgacgttatcggttagtgagacaggcccctttt

SEQ ID NO: 376 - DNA, 70 bp, Artificial Sequence
  Description of Artificial Sequence Synthetic primer
aggaaaaggggcctgtctcaccaacatggttgacctggtctcatacaccatcttttatccaaagataccc

SEQ ID NO: 377 - DNA, 70 bp, Artificial Sequence
  Description of Artificial Sequence Synthetic primer
gggtatctttggataaaagatggtgtatgagaccaggtcaaccatgttggtgagacaggccccttttcct

SEQ ID NO: 378 - DNA, 82 bp, Artificial Sequence
  Description of Artificial Sequence Synthetic primer
aggaaaaggggcctgtctcaaccgataacgtcggtgtttctgaacttgatccacttccactttcttttatccaaagataccc

SEQ ID NO: 379 - DNA, 82 bp, Artificial Sequence
  Description of Artificial Sequence Synthetic primer
gggtatctttggataaaagaaagtggaagtggatcaagttcagaaacaccgacgttatcggttgagacaggccccttttcct

SEQ ID NO: 380 - DNA, 70 bp, Artificial Sequence
  Description of Artificial Sequence Synthetic primer
aggaaaaggggcctgtctcaccaacatggttcaccgattctgtaacaccatcttttatccaaagataccc

SEQ ID NO: 381 - DNA, 70 bp, Artificial Sequence
  Description of Artificial Sequence Synthetic primer
gggtatctttggataaaagatggtgttacagaatcggtgaaccatgttggtgagacaggccccttttcct

SEQ ID NO: 382 - DNA, 79 bp, Artificial Sequence
  Description of Artificial Sequence Synthetic primer
aggaaaaggggcctgtctcaaccgaatggttggttcttttcgttgtttctccatctgaatcttttatccaaagataccc

SEQ ID NO: 383 - DNA, 79 bp, Artificial Sequence
  Description of Artificial Sequence Synthetic primer
gggtatctttggataaaagattcagatggagaaacaacgaaaagaaccaaccattcggttgagacaggccccttttcct

SEQ ID NO: 384 - DNA, 76 bp, Artificial Sequence
  Description of Artificial Sequence Synthetic primer
aaaaggggcctgtctcactaaactggagagaatctaccgtatctggtccacatccaagcttcagcctctcttttat

SEQ ID NO: 385 - DNA, 76 bp, Artificial Sequence
  Description of Artificial Sequence Synthetic primer
aggaaaaggggcctgtctcaaactggagagaatctaccgtatctggtccacatccatcttttatccaaagataccc

SEQ ID NO: 386 - DNA, 76 bp, Artificial Sequence
  Description of Artificial Sequence Synthetic primer
ataaaagagaggctgaagcttggatgtggaccagatacggtagattctctccagtttagtgagacaggcccctttt

SEQ ID NO: 387 - DNA, 76 bp, Artificial Sequence
  Description of Artificial Sequence Synthetic primer
gggtatctttggataaaagatggatgtggaccagatacggtagattctctccagtttgagacaggccccttttcct

SEQ ID NO: 388 - DNA, 70 bp, Artificial Sequence
  Description of Artificial Sequence Synthetic primer
aggaaaaggggcctgtctcaaccacatggttgacctggtctccaacagaatcttttatccaaagataccc

SEQ ID NO: 389 - DNA, 70 bp, Artificial Sequence
  Description of Artificial Sequence Synthetic primer
gggtatctttggataaaagattctgttggagaccaggtcaaccatgtggttgagacaggccccttttcct

SEQ ID NO: 390 - DNA, 76 bp, Artificial Sequence
  Description of Artificial Sequence Synthetic primer
aaaaggggcctgtctcactagaacattggttgacctgggtccaattcgatgaagtgagcttcagcctctcttttat

SEQ ID NO: 391 - DNA, 76 bp, Artificial Sequence
  Description of Artificial Sequence Synthetic primer
aggaaaaggggcctgtctcagaacattggttgacctgggtccaattcgatgaagtgtcttttatccaaagataccc

SEQ ID NO: 392 - DNA, 76 bp, Artificial Sequence
  Description of Artificial Sequence Synthetic primer
ataaaagagaggctgaagctcacttcatcgaattggacccaggtcaaccaatgttctagtgagacaggcccctttt

SEQ ID NO: 393 - DNA, 76 bp, Artificial Sequence
  Description of Artificial Sequence Synthetic primer
gggtatctttggataaaagacacttcatcgaattggacccaggtcaaccaatgttctgagacaggccccttttcct

SEQ ID NO: 394 - DNA, 70 bp, Artificial Sequence
  Description of Artificial Sequence Synthetic primer
aaaaggggcctgtctcactaccaacatggttcaccgattctgtaacaccaagcttcagcctctcttttat

SEQ ID NO: 395 - DNA, 70 bp, Artificial Sequence
  Description of Artificial Sequence Synthetic primer
ataaaagagaggctgaagcttggtgttacagaatcggtgaaccatgttggtagtgagacaggcccctttt

SEQ ID NO: 396 - DNA, 79 bp, Artificial Sequence
  Description of Artificial Sequence Synthetic primer
aaaaggggcctgtctcactaaccgaatggttggttcttttcgttgtttctccatctgaaagcttcagcctctcttttat

SEQ ID NO: 397 - DNA, 79 bp, Artificial Sequence
  Description of Artificial Sequence Synthetic primer
ataaaagagaggctgaagctttcagatggagaaacaacgaaaagaaccaaccattcggttagtgagacaggcccctttt

SEQ ID NO: 398 - DNA, 67 bp, Artificial Sequence
  Description of Artificial Sequence Synthetic primer
aaaaggggcctgtctcactaacaaccttgacctggtctggtacaccaagcttcagcctctcttttat

SEQ ID NO: 399 - DNA, 67 bp, Artificial Sequence
  Description of Artificial Sequence Synthetic primer
ataaaagagaggctgaagcttggtgtaccagaccaggtcaaggttgttagtgagacaggcccctttt

SEQ ID NO: 400 - DNA, 70 bp, Artificial Sequence
  Description of Artificial Sequence Synthetic primer
aaaaggggcctgtctcactaaccacatggttgacctggtctccaacagaaagcttcagcctctcttttat

SEQ ID NO: 401 - DNA, 70 bp, Artificial Sequence
  Description of Artificial Sequence Synthetic primer
ataaaagagaggctgaagctttctgttggagaccaggtcaaccatgtggttagtgagacaggcccctttt

SEQ ID NO: 402 - DNA, 79 bp, Artificial Sequence
  Description of Artificial Sequence Synthetic primer
aaaaggggcctgtctcactagtacattggttgacctggcttcaattgcaaccagtgccaagcttcagcctctcttttat

SEQ ID NO: 403 - DNA, 79 bp, Artificial Sequence
  Description of Artificial Sequence Synthetic primer
ataaaagagaggctgaagcttggcactggttgcaattgaagccaggtcaaccaatgtactagtgagacaggcccctttt

SEQ ID NO: 404 - DNA, 79 bp, Artificial Sequence
  Description of Artificial Sequence Synthetic primer
aaaaggggcctgtctcactagtagattggttgaccgttgtccaattccaaccagtgccaagcttcagcctctcttttat

SEQ ID NO: 405 - DNA, 79 bp, Artificial Sequence
  Description of Artificial Sequence Synthetic primer
ataaaagagaggctgaagcttggcactggttggaattggacaacggtcaaccaatctactagtgagacaggcccctttt

SEQ ID NO: 406 - DNA, 67 bp, Artificial Sequence
  Description of Artificial Sequence Synthetic primer
aggaaaaggggcctgtctcaacatggttgacctggtctaccacaccatcttttatccaaagataccc

SEQ ID NO: 407 - DNA, 67 bp, Artificial Sequence
  Description of Artificial Sequence Synthetic primer
gggtatctttggataaaagatggtgtggtagaccaggtcaaccatgttgagacaggccccttttcct

SEQ ID NO: 408 - DNA, 79 bp, Artificial Sequence
  Description of Artificial Sequence Synthetic primer
aggaaaaggggcctgtctcaacctggttcgaagtaaccgaagttggtcaatctgaaacctcttttatccaaagataccc

SEQ ID NO: 409 - DNA, 79 bp, Artificial Sequence
  Description of Artificial Sequence Synthetic primer
gggtatctttggataaaagaggtttcagattgaccaacttcggttacttcgaaccaggttgagacaggccccttttcct

SEQ ID NO: 410 - DNA, 67 bp, Artificial Sequence
  Description of Artificial Sequence Synthetic primer
aggaaaaggggcctgtctcaacaaccttgacctggtctggtacaccatcttttatccaaagataccc

SEQ ID NO: 411 - DNA, 67 bp, Artificial Sequence
  Description of Artificial Sequence Synthetic primer
gggtatctttggataaaagatggtgtaccagaccaggtcaaggttgttgagacaggccccttttcct

SEQ ID NO: 412 - DNA, 79 bp, Artificial Sequence
  Description of Artificial Sequence Synthetic primer
aggaaaaggggcctgtctcagtacattggttgacctggcttcaattgcaaccagtgccatcttttatccaaagataccc

SEQ ID NO: 413 - DNA, 79 bp, Artificial Sequence
  Description of Artificial Sequence Synthetic primer
gggtatctttggataaaagatggcactggttgcaattgaagccaggtcaaccaatgtactgagacaggccccttttcct

SEQ ID NO: 414 - DNA, 79 bp, Artificial Sequence
  Description of Artificial Sequence Synthetic primer
aggaaaaggggcctgtctcagtagattggttgaccgttgtccaattccaaccagtgccatcttttatccaaagataccc

SEQ ID NO: 415 - DNA, 79 bp, Artificial Sequence
  Description of Artificial Sequence Synthetic primer
gggtatctttggataaaagatggcactggttggaattggacaacggtcaaccaatctactgagacaggccccttttcct

SEQ ID NO: 416 - DNA, 60 bp, Artificial Sequence
  Description of Artificial Sequence Synthetic primer
gctatttctagctctaaaacgaagacacctttgataatatgatcatttatctttcactgc

SEQ ID NO: 417 - DNA, 60 bp, Artificial Sequence
  Description of Artificial Sequence Synthetic primer
gcagtgaaagataaatgatcatattatcaaaggtgtcttcgttttagagctagaaatagc

SEQ ID NO: 418 - DNA, 20 bp, Artificial Sequence
  Description of Artificial Sequence Synthetic primer
ctgctacggttggcccatac

SEQ ID NO: 419 - DNA, 21 bp, Artificial Sequence
  Description of Artificial Sequence Synthetic primer
acttcacggtaggtggtaagc

SEQ ID NO: 420 - DNA, 60 bp, Artificial Sequence
  Description of Artificial Sequence Synthetic primer
gctatttctagctctaaaactcttttcactgctggtctttgatcatttatctttcactgc

SEQ ID NO: 421 - DNA, 60 bp, Artificial Sequence
  Description of Artificial Sequence Synthetic primer
gcagtgaaagataaatgatcaaagaccagcagtgaaaagagttttagagctagaaatagc

SEQ ID NO: 422 - DNA, 78 bp, Artificial Sequence
  Description of Artificial Sequence Synthetic primer
aagataaaggagggagaacaacgtttttgtacgcagaaattctattcgatggctttgtacttattttggttttatccg

SEQ ID NO: 423 - DNA, 78 bp, Artificial Sequence
  Description of Artificial Sequence Synthetic primer
tcggataaaaccaaaataagtacaaagccatcgaatagaatttctgcgtacaaaaacgttgttctccctcctttatct

SEQ ID NO: 424 - DNA, 24 bp, Artificial Sequence
  Description of Artificial Sequence Synthetic primer
ttccatccacttcttctgtcgttc

SEQ ID NO: 425 - DNA, 25 bp, Artificial Sequence
  Description of Artificial Sequence Synthetic primer
gggtggttcatctttcatttcctgc

SEQ ID NO: 426 - DNA, 60 bp, Artificial Sequence
  Description of Artificial Sequence Synthetic primer
gctatttctagctctaaaactctgagtggcttgtgtggaactgccaatcgcagctcccag

SEQ ID NO: 427 - DNA, 60 bp, Artificial Sequence
  Description of Artificial Sequence Synthetic primer
ctgggagctgcgattggcagttccacacaagccactcagagttttagagctagaaatagc

SEQ ID NO: 428 - DNA, 60 bp, Artificial Sequence
  Description of Artificial Sequence Synthetic primer
gctatttctagctctaaaactctgagtggcttgtgtggaagatcatttatctttcactgc

SEQ ID NO: 429 - DNA, 60 bp, Artificial Sequence
  Description of Artificial Sequence Synthetic primer
gcagtgaaagataaatgatcttccacacaagccactcagagttttagagctagaaatagc

SEQ ID NO: 430 - DNA, 79 bp, Artificial Sequence
  Description of Artificial Sequence Synthetic primer
agggtagatattgatttgacctcttggttgtcgtcaaaaataaggttggtagttattgttgtatgaagatgatagctcg

SEQ ID NO: 431 - DNA, 78 bp, Artificial Sequence
  Description of Artificial Sequence Synthetic primer
gcgagctatcatcttcatacaacaataactaccaaccttatttttgacgacaaccaagaggtcaaatcaatatctacc

SEQ ID NO: 432 - DNA, 23 bp, Artificial Sequence
  Description of Artificial Sequence Synthetic primer
tgcgctaaatagacatcccgttc

SEQ ID NO: 433 - DNA, 24 bp, Artificial Sequence
  Description of Artificial Sequence Synthetic primer
cagaggcatcataatcagggagtg

SEQ ID NO: 434 - DNA, 60 bp, Artificial Sequence
  Description of Artificial Sequence Synthetic primer
gctatttctagctctaaaacggttttaactgcaaccaatgctgccaatcgcagctcccag

SEQ ID NO: 435 - DNA, 60 bp, Artificial Sequence
  Description of Artificial Sequence Synthetic primer
ctgggagctgcgattggcagcattggttgcagttaaaaccgttttagagctagaaatagc

SEQ ID NO: 436 - DNA, 60 bp, Artificial Sequence
  Description of Artificial Sequence Synthetic primer
gctatttctagctctaaaactcaatttttactgcagttttgatcatttatctttcactgc

SEQ ID NO: 437 - DNA, 60 bp, Artificial Sequence
  Description of Artificial Sequence Synthetic primer
gcagtgaaagataaatgatcaaaactgcagtaaaaattgagttttagagctagaaatagc

SEQ ID NO: 438 - DNA, 79 bp, Artificial Sequence
  Description of Artificial Sequence Synthetic primer
gtcgactttgttacatctacactgttgttatcagtcgggctcttttaatcgtttatattgtgtatgaaattgatagttt

SEQ ID NO: 439 - DNA, 79 bp, Artificial Sequence
  Description of Artificial Sequence Synthetic primer
caaactatcaatttcatacacaatataaacgattaaaagagcccgactgataacaacagtgtagatgtaacaaagtcga

SEQ ID NO: 440 - DNA, 20 bp, Artificial Sequence
  Description of Artificial Sequence Synthetic primer
ggcgacgcctgtagtgattg

SEQ ID NO: 441 - DNA, 21 bp, Artificial Sequence
  Description of Artificial Sequence Synthetic primer
gggaaccttgcttgcagacag

SEQ ID NO: 442 - DNA, 60 bp, Artificial Sequence
  Description of Artificial Sequence Synthetic primer
gctatttctagctctaaaacggcttgagttgcaaccagtgctgccaatcgcagctcccag

SEQ ID NO: 443 - DNA, 60 bp, Artificial Sequence
  Description of Artificial Sequence Synthetic primer
ctgggagctgcgattggcagcactggttgcaactcaagccgttttagagctagaaatagc

SEQ ID NO: 444 - DNA, 60 bp, Artificial Sequence
  Description of Artificial Sequence Synthetic primer
gctatttctagctctaaaacttctcacttttattttagcggatcatttatctttcactgc

SEQ ID NO: 445 - DNA, 60 bp, Artificial Sequence
  Description of Artificial Sequence Synthetic primer
gcagtgaaagataaatgatccgctaaaataaaagtgagaagttttagagctagaaatagc

SEQ ID NO: 446 - DNA, 79 bp, Artificial Sequence
  Description of Artificial Sequence Synthetic primer
aagaaatcgagagggtttagaagtagtttagggtcatttttttctccaatatgtgaatttactggaatttgatgcaggt

SEQ ID NO: 447 - DNA, 79 bp, Artificial Sequence
  Description of Artificial Sequence Synthetic primer
cacctgcatcaaattccagtaaattcacatattggagaaaaaaatgaccctaaactacttctaaaccctctcgatttct

SEQ ID NO: 448 - DNA, 51 bp, Artificial Sequence
  Description of Artificial Sequence Synthetic primer
gtgcaattgtacctgaagatgagtaagactctcaatgaaaccacttacaac

SEQ ID NO: 449 - DNA, 52 bp, Artificial Sequence
  Description of Artificial Sequence Synthetic primer
gttataggttcaatttggtaattaaagatagagttgtaagtggtttcattga

SEQ ID NO: 450 - DNA, 24 bp, Artificial Sequence
  Description of Artificial Sequence Synthetic primer
tgactaggacttggatttggttgc

SEQ ID NO: 451 - DNA, 22 bp, Artificial Sequence
  Description of Artificial Sequence Synthetic primer
gcgctcacgttagtcacatctc

SEQ ID NO: 452 - DNA, 60 bp, Artificial Sequence
  Description of Artificial Sequence Synthetic primer
gctatttctagctctaaaacgtcagacgtatacaaagatgctgccaatcgcagctcccag

SEQ ID NO: 453 - DNA, 60 bp, Artificial Sequence
  Description of Artificial Sequence Synthetic primer
ctgggagctgcgattggcagcatctttgtatacgtctgacgttttagagctagaaatagc

SEQ ID NO: 454 - DNA, 60 bp, Artificial Sequence
  Description of Artificial Sequence Synthetic primer
gctatttctagctctaaaacatttttatccaccatcttacgatcatttatctttcactgc

SEQ ID NO: 455 - DNA, 60 bp, Artificial Sequence
  Description of Artificial Sequence Synthetic primer
gcagtgaaagataaatgatcgtaagatggtggataaaaatgttttagagctagaaatagc

SEQ ID NO: 456 - DNA, 20 bp, Artificial Sequence
  Description of Artificial Sequence Synthetic primer
actcttcgcggtcaggtctc

SEQ ID NO: 457 - DNA, 30 bp, Artificial Sequence
  Description of Artificial Sequence Synthetic primer
ggcaatactacgttggtatcaaaatagtgg

SEQ ID NO: 458 - DNA, 60 bp, Artificial Sequence
  Description of Artificial Sequence Synthetic primer
gctatttctagctctaaaactcgattggtatctacctcaactgccaatcgcagctcccag

SEQ ID NO: 459 - DNA, 60 bp, Artificial Sequence
  Description of Artificial Sequence Synthetic primer
ctgggagctgcgattggcagttgaggtagataccaatcgagttttagagctagaaatagc

SEQ ID NO: 460 - DNA, 60 bp, Artificial Sequence
  Description of Artificial Sequence Synthetic primer
gctatttctagctctaaaacctgttctactattggttattgatcatttatctttcactgc

SEQ ID NO: 461 - DNA, 60 bp, Artificial Sequence
  Description of Artificial Sequence Synthetic primer
gcagtgaaagataaatgatcaataaccaatagtagaacaggttttagagctagaaatagc

SEQ ID NO: 462 - DNA, 79 bp, Artificial Sequence
  Description of Artificial Sequence Synthetic primer
tttttaattcttgtatcataaattcaaaaattatattataccttggtgaacaagacaattcaaataaagaaagcggttc

SEQ ID NO: 463 - DNA, 79 bp, Artificial Sequence
  Description of Artificial Sequence Synthetic primer
ggaaccgctttctttatttgaattgtcttgttcaccaaggtataatataatttttgaatttatgatacaagaattaaaa

SEQ ID NO: 464 - DNA, 20 bp, Artificial Sequence
  Description of Artificial Sequence Synthetic primer
taggacctgtgcctggcaag

SEQ ID NO: 465 - DNA, 26 bp, Artificial Sequence
  Description of Artificial Sequence Synthetic primer
catcacaatatactagcagtggcacc

SEQ ID NO: 466 - DNA, 60 bp, Artificial Sequence
  Description of Artificial Sequence Synthetic primer
gctatttctagctctaaaacgaactttctggcttcctcatctgccaatcgcagctcccag

SEQ ID NO: 467 - DNA, 60 bp, Artificial Sequence
  Description of Artificial Sequence Synthetic primer
ctgggagctgcgattggcagatgaggaagccagaaagttcgttttagagctagaaatagc

SEQ ID NO: 468 - DNA, 60 bp, Artificial Sequence
  Description of Artificial Sequence Synthetic primer
gctatttctagctctaaaaccatcagacatttttgattctgatcatttatctttcactgc

SEQ ID NO: 469 - DNA, 60 bp, Artificial Sequence
  Description of Artificial Sequence Synthetic primer
gcagtgaaagataaatgatcagaatcaaaaatgtctgatggttttagagctagaaatagc

SEQ ID NO: 470 - DNA, 79 bp, Artificial Sequence
  Description of Artificial Sequence Synthetic primer
gaaggtcacgaaattactttttcaaagccgtaaattttgattttgattcttggatatggttcttaacggtgcattttta

SEQ ID NO: 471 - DNA, 79 bp, Artificial Sequence
  Description of Artificial Sequence Synthetic primer
ttaaaaatgcaccgttaagaaccatatccaagaatcaaaatcaaaatttacggctttgaaaaagtaatttcgtgacctt

SEQ ID NO: 472 - DNA, 24 bp, Artificial Sequence
  Description of Artificial Sequence Synthetic primer
tgcgtttcatttggccgttatcac

SEQ ID NO: 473 - DNA, 26 bp, Artificial Sequence
  Description of Artificial Sequence Synthetic primer
cttggtgtgcagaatagtgatagagc

SEQ ID NO: 474 - DNA, 60 bp, Artificial Sequence
  Description of Artificial Sequence Synthetic primer
gctatttctagctctaaaacgcagtattttctgaactatgctgccaatcgcagctcccag

SEQ ID NO: 475 - DNA, 60 bp, Artificial Sequence
  Description of Artificial Sequence Synthetic primer
ctgggagctgcgattggcagcatagttcagaaaatactgcgttttagagctagaaatagc

SEQ ID NO: 476 - DNA, 60 bp, Artificial Sequence
  Description of Artificial Sequence Synthetic primer
gctatttctagctctaaaactattattgctgacttgtatggatcatttatctttcactgc

SEQ ID NO: 477 - DNA, 60 bp, Artificial Sequence
  Description of Artificial Sequence Synthetic primer
gcagtgaaagataaatgatccatacaagtcagcaataatagttttagagctagaaatagc

SEQ ID NO: 478 - DNA, 79 bp, Artificial Sequence
  Description of Artificial Sequence Synthetic primer
aatactcctagtccagtaaatataatgcgacactcttgtggaaaattttgatagtattttgcctttcctacacaaattt

SEQ ID NO: 479 - DNA, 79 bp, Artificial Sequence
  Description of Artificial Sequence Synthetic primer
taaatttgtgtaggaaaggcaaaatactatcaaaattttccacaagagtgtcgcattatatttactggactaggagtat

SEQ ID NO: 480 - DNA, 53 bp, Artificial Sequence
  Description of Artificial Sequence Synthetic primer
accaagaacttagtttcgacggatactagtaaaatgtctgatgcggctccttc

SEQ ID NO: 481 - DNA, 60 bp, Artificial Sequence
  Description of Artificial Sequence Synthetic primer
acgaaattactttttcaaagccgtctcgagctataaattattattatcttcagtccagaa

SEQ ID NO: 482 - DNA, 41 bp, Artificial Sequence
  Description of Artificial Sequence Synthetic primer
gtgtcgtctagaaaaatgaatatcaattcaactttcatacc

SEQ ID NO: 483 - DNA, 36 bp, Artificial Sequence
  Description of Artificial Sequence Synthetic primer
gcaagtctcgagctacactcttttgatggtgatttg

SEQ ID NO: 484 - DNA, 54 bp, Artificial Sequence
  Description of Artificial Sequence Synthetic primer
accaagaacttagtttcgacggatactagtaaaatgggtgaagaggtatctagc

SEQ ID NO: 485 - DNA, 51 bp, Artificial Sequence
  Description of Artificial Sequence Synthetic primer
acgaaattactttttcaaagccgtctcgagctagttgcaatcacttccggt

SEQ ID NO: 486 - DNA, 57 bp, Artificial Sequence
  Description of Artificial Sequence Synthetic primer
accaagaacttagtttcgacggatactagtaaaatggcttctaactcttctaacttc

SEQ ID NO: 487 - DNA, 53 bp, Artificial Sequence
  Description of Artificial Sequence Synthetic primer
acgaaattactttttcaaagccgtctcgagctaagccttttgaacaccgtaag

SEQ ID NO: 488 - DNA, 53 bp, Artificial Sequence
  Description of Artificial Sequence Synthetic primer
accaagaacttagtttcgacggatactagtaaaatggagatgggctacgatcc

SEQ ID NO: 489 - DNA, 54 bp, Artificial Sequence
  Description of Artificial Sequence Synthetic primer
acgaaattactttttcaaagccgtctcgagctatttgtcacactgactttgttg

SEQ ID NO: 490 - DNA, 57 bp, Artificial Sequence
  Description of Artificial Sequence Synthetic primer
accaagaacttagtttcgacggatactagtaaaatgtctaaggaagttttcgaccca

SEQ ID NO: 491 - DNA, 54 bp, Artificial Sequence
  Description of Artificial Sequence Synthetic primer
acgaaattactttttcaaagccgtctcgagctacaatggagctctgattctttc

SEQ ID NO: 492 - DNA, 57 bp, Artificial Sequence
  Description of Artificial Sequence Synthetic primer
accaagaacttagtttcgacggatactagtaaaatgtcagaagagatacccagtttg

SEQ ID NO: 493 - DNA, 58 bp, Artificial Sequence
  Description of Artificial Sequence Synthetic primer
acgaaattactttttcaaagccgtctcgagctatcttaattctttgaatacggttttc

SEQ ID NO: 494 - DNA, 57 bp, Artificial Sequence
  Description of Artificial Sequence Synthetic primer
accaagaacttagtttcgacggatactagtaaaatggacgaagcaatcaatgcaaac

SEQ ID NO: 495 - DNA, 54 bp, Artificial Sequence
  Description of Artificial Sequence Synthetic primer
acgaaattactttttcaaagccgtctcgagctattttttcaacatagtcacttc

SEQ ID NO: 496 - DNA, 56 bp, Artificial Sequence
  Description of Artificial Sequence Synthetic primer
accaagaacttagtttcgacggatactagtaaaatggaccaaactttgtctgctac

SEQ ID NO: 497 - DNA, 56 bp, Artificial Sequence
  Description of Artificial Sequence Synthetic primer
acgaaattactttttcaaagccgtctcgagctacaatctttcttctcttctttcga

SEQ ID NO: 498 - DNA, 52 bp, Artificial Sequence
  Description of Artificial Sequence Synthetic primer
accaagaacttagtttcgacggatactagtaaaatggcaccctcattcgacc

SEQ ID NO: 499 - DNA, 51 bp, Artificial Sequence
  Description of Artificial Sequence Synthetic primer
acgaaattactttttcaaagccgtctcgagctaggcctttgtgccagcttc

SEQ ID NO: 500 - DNA, 55 bp, Artificial Sequence
  Description of Artificial Sequence Synthetic primer
accaagaacttagtttcgacggatactagtaaaatgagacaaccatggtggaaag

SEQ ID NO: 501 - DNA, 57 bp, Artificial Sequence
  Description of Artificial Sequence Synthetic primer
acgaaattactttttcaaagccgtctcgagctacgtccactttttagtttcagattc

SEQ ID NO: 502 - DNA, 54 bp, Artificial Sequence
  Description of Artificial Sequence Synthetic primer
accaagaacttagtttcgacggatactagtaaaatgagttcccaatcacaccca

SEQ ID NO: 503 - DNA, 56 bp, Artificial Sequence
  Description of Artificial Sequence Synthetic primer
acgaaattactttttcaaagccgtctcgagctatgaagtccttgtgatatcgttac

SEQ ID NO: 504 - DNA, 57 bp, Artificial Sequence
  Description of Artificial Sequence Synthetic primer
accaagaacttagtttcgacggatactagtaaaatgtcaggaattgatgatatgggt

SEQ ID NO: 505 - DNA, 59 bp, Artificial Sequence
  Description of Artificial Sequence Synthetic primer
acgaaattactttttcaaagccgtctcgagctattgttttctaaatgttattctttttg

SEQ ID NO: 506 - DNA, 56 bp, Artificial Sequence
  Description of Artificial Sequence Synthetic primer
accaagaacttagtttcgacggatactagtaaaatgtctggtttggctaacaacac

SEQ ID NO: 507 - DNA, 55 bp, Artificial Sequence
  Description of Artificial Sequence Synthetic primer
acgaaattactttttcaaagccgtctcgagctaccatttgacgttcttcttcaaa

SEQ ID NO: 508 - DNA, 60 bp, Artificial Sequence
  Description of Artificial Sequence Synthetic primer
accaagaacttagtttcgacggatactagtaaaatgagtgagattaacaattctacctac

SEQ ID NO: 509 - DNA, 60 bp, Artificial Sequence
  Description of Artificial Sequence Synthetic primer
acgaaattactttttcaaagccgtctcgagctataatttctttaggataatttttttact

SEQ ID NO: 510 - DNA, 65 bp, Artificial Sequence
  Description of Artificial Sequence Synthetic primer
acaccaagaacttagtttcgacggatactagtaaaatggatactagtatcaatactctcaaccct

SEQ ID NO: 511 - DNA, 58 bp, Artificial Sequence
  Description of Artificial Sequence Synthetic primer
acgaaattactttttcaaagccgtctcgagctagctttcagaaaagtgagaggtcgtt

SEQ ID NO: 512 - DNA, 54 bp, Artificial Sequence
  Description of Artificial Sequence Synthetic primer
accaagaacttagtttcgacggatactagtaaaatgtactcctgggacgaattc

SEQ ID NO: 513 - DNA, 54 bp, Artificial Sequence
  Description of Artificial Sequence Synthetic primer
acgaaattactttttcaaagccgtctcgagctatggcaaagtttcttcggtctt

SEQ ID NO: 514 - DNA, 52 bp, Artificial Sequence
  Description of Artificial Sequence Synthetic primer
accaagaacttagtttcgacggatactagtaaaatgtctgacgctccaccac

SEQ ID NO: 515 - DNA, 54 bp, Artificial Sequence
  Description of Artificial Sequence Synthetic primer
acgaaattactttttcaaagccgtctcgagctattgcttcttgacggtgatctt

SEQ ID NO: 516 - DNA, 54 bp, Artificial Sequence
  Description of Artificial Sequence Synthetic primer
accaagaacttagtttcgacggatactagtaaaatggcttctatggttccacca

SEQ ID NO: 517 - DNA, 54 bp, Artificial Sequence
  Description of Artificial Sequence Synthetic primer
acgaaattactttttcaaagccgtctcgagctagacgatggagttgttacgttg

SEQ ID NO: 518 - DNA, 54 bp, Artificial Sequence
  Description of Artificial Sequence Synthetic primer
accaagaacttagtttcgacggatactagtaaaatggtggtaacagctccacct

SEQ ID NO: 519 - DNA, 53 bp, Artificial Sequence
  Description of Artificial Sequence Synthetic primer
acgaaattactttttcaaagccgtctcgagctagtcggaacggactgagtatg

SEQ ID NO: 520 - DNA, 53 bp, Artificial Sequence
  Description of Artificial Sequence Synthetic primer
accaagaacttagtttcgacggatactagtaaaatgaagtcctgctccatcgg

SEQ ID NO: 521 - DNA, 53 bp, Artificial Sequence
  Description of Artificial Sequence Synthetic primer
acgaaattactttttcaaagccgtctcgagctagatggaggtggagtcgatca

SEQ ID NO: 522 - DNA, 54 bp, Artificial Sequence
  Description of Artificial Sequence Synthetic primer
accaagaacttagtttcgacggatactagtaaaatggacatcaacaacaccatc

SEQ ID NO: 523 - DNA, 53 bp, Artificial Sequence
  Description of Artificial Sequence Synthetic primer
acgaaattactttttcaaagccgtctcgagctagaccttcttgtaggtgactt

SEQ ID NO: 524 - DNA, 56 bp, Artificial Sequence
  Description of Artificial Sequence Synthetic primer
accaagaacttagtttcgacggatactagtaaaatgaacaagattgtctccaagtt

SEQ ID NO: 525 - DNA, 53 bp, Artificial Sequence
  Description of Artificial Sequence Synthetic primer
acgaaattactttttcaaagccgtctcgagctattggttgttgtgagcggtct

SEQ ID NO: 526 - DNA, 54 bp, Artificial Sequence
  Description of Artificial Sequence Synthetic primer
accaagaacttagtttcgacggatactagtaaaatgcgtgaaccatggtggaag

SEQ ID NO: 527 - DNA, 54 bp, Artificial Sequence
  Description of Artificial Sequence Synthetic primer
acgaaattactttttcaaagccgtctcgagctatggccacttcttgatttcggt

SEQ ID NO: 528 - DNA, 53 bp, Artificial Sequence
  Description of Artificial Sequence Synthetic primer
acgaaattactttttcaaagccgtctcgagctaacctctttcaccgacttcac

SEQ ID NO: 529 - DNA, 54 bp, Artificial Sequence
  Description of Artificial Sequence Synthetic primer
accaagaacttagtttcgacggatactagtaaaatggctgctagaattatccca

SEQ ID NO: 530 - DNA, 54 bp, Artificial Sequence
  Description of Artificial Sequence Synthetic primer
acgaaattactttttcaaagccgtctcgagctagaccatgttttcagaaccaac

SEQ ID NO: 531 - DNA, 54 bp, Artificial Sequence
  Description of Artificial Sequence Synthetic primer
accaagaacttagtttcgacggatactagtaaaatggccgaagactccatcttc

SEQ ID NO: 532 - DNA, 52 bp, Artificial Sequence
  Description of Artificial Sequence Synthetic primer
acgaaattactttttcaaagccgtctcgagctacttacgggtgacgtcggtt

SEQ ID NO: 533 - DNA, 54 bp, Artificial Sequence
  Description of Artificial Sequence Synthetic primer
accaagaacttagtttcgacggatactagtaaaatgtccggtaagcaagacttg

SEQ ID NO: 534 - DNA, 54 bp, Artificial Sequence
  Description of Artificial Sequence Synthetic primer
acgaaattactttttcaaagccgtctcgagctaggtggtcatcaagatcttgga

SEQ ID NO: 535 - DNA, 54 bp, Artificial Sequence
  Description of Artificial Sequence Synthetic primer
accaagaacttagtttcgacggatactagtaaaatggctacccacaaccaaatc

SEQ ID NO: 536 - DNA, 54 bp, Artificial Sequence
  Description of Artificial Sequence Synthetic primer
acgaaattactttttcaaagccgtctcgagctagacgtcaaaagattcacgacg

SEQ ID NO: 537 - DNA, 54 bp, Artificial Sequence
  Description of Artificial Sequence Synthetic primer
accaagaacttagtttcgacggatactagtaaaatggactctaagttcgaccca

SEQ ID NO: 538 - DNA, 54 bp, Artificial Sequence
  Description of Artificial Sequence Synthetic primer
acgaaattactttttcaaagccgtctcgagctacaatctttgacaggagtggac

SEQ ID NO: 539 - DNA, 54 bp, Artificial Sequence
  Description of Artificial Sequence Synthetic primer
accaagaacttagtttcgacggatactagtaaaatggatggttcttctgctcca

SEQ ID NO: 540 - DNA, 54 bp, Artificial Sequence
  Description of Artificial Sequence Synthetic primer
acgaaattactttttcaaagccgtctcgagctaggcgaagttatcacgttgcat

SEQ ID NO: 541 - DNA, 54 bp, Artificial Sequence
  Description of Artificial Sequence Synthetic primer
accaagaacttagtttcgacggatactagtaaaatgaacccagctgacatcaac

SEQ ID NO: 542 - DNA, 54 bp, Artificial Sequence
  Description of Artificial Sequence Synthetic primer
acgaaattactttttcaaagccgtctcgagagctatcaatctatgggtggtgac

SEQ ID NO: 543 - DNA, 55 bp, Artificial Sequence
  Description of Artificial Sequence Synthetic primer
accaagaacttagtttcgacggatactagtaaaatggactcctacttgttgaacc

SEQ ID NO: 544 - DNA, 54 bp, Artificial Sequence
  Description of Artificial Sequence Synthetic primer
acgaaattactttttcaaagccgtctcgagctacttcataccgatgtcggtgtt

SEQ ID NO: 545 - DNA, 54 bp, Artificial Sequence
  Description of Artificial Sequence Synthetic primer
accaagaacttagtttcgacggatactagtaaaatgaactccaccttcgaccca

SEQ ID NO: 546 - DNA, 54 bp, Artificial Sequence
  Description of Artificial Sequence Synthetic primer
acgaaattactttttcaaagccgtctcgagctaaatatcaccgtgggcgtcctt

SEQ ID NO: 547 - DNA, 54 bp, Artificial Sequence
  Description of Artificial Sequence Synthetic primer
accaagaacttagtttcgacggatactagtaaaatgtccactgccaacgttcat

SEQ ID NO: 548 - DNA, 54 bp, Artificial Sequence
  Description of Artificial Sequence Synthetic primer
acgaaattactttttcaaagccgtctcgagctagaagatgtcctctctctcgat

SEQ ID NO: 549 - DNA, 54 bp, Artificial Sequence
  Description of Artificial Sequence Synthetic primer
accaagaacttagtttcgacggatactagtaaaatgtcttccttcgacccatac

SEQ ID NO: 550 - DNA, 54 bp, Artificial Sequence
  Description of Artificial Sequence Synthetic primer
acgaaattactttttcaaagccgtctcgagctaagaggaagaagtgttggcgat

SEQ ID NO: 551 - DNA, 54 bp, Artificial Sequence
  Description of Artificial Sequence Synthetic primer
accaagaacttagtttcgacggatactagtaaaatggagcaaatcccagtctac

SEQ ID NO: 552 - DNA, 54 bp, Artificial Sequence
  Description of Artificial Sequence Synthetic primer
acgaaattactttttcaaagccgtctcgagctaggcgaattcgaaacctctttc

SEQ ID NO: 553 - DNA, 54 bp, Artificial Sequence
  Description of Artificial Sequence Synthetic primer
accaagaacttagtttcgacggatactagtaaaatggaccacaacacccaacac

SEQ ID NO: 554 - DNA, 53 bp, Artificial Sequence
  Description of Artificial Sequence Synthetic primer
acgaaattactttttcaaagccgtctcgagctagtcatcgtggtcaccaacgt

SEQ ID NO: 555 - DNA, 52 bp, Artificial Sequence
  Description of Artificial Sequence Synthetic primer
accaagaacttagtttcgacggatactagtaaaatgaaacccgccgctggac

SEQ ID NO: 556 - DNA, 53 bp, Artificial Sequence
  Description of Artificial Sequence Synthetic primer
acgaaattactttttcaaagccgtctcgagctagaccatgtcccttctgacct

SEQ ID NO: 557 - DNA, 54 bp, Artificial Sequence
  Description of Artificial Sequence Synthetic primer
accaagaacttagtttcgacggatactagtaaaatgcaattgccaccacgtcca

SEQ ID NO: 558 - DNA, 56 bp, Artificial Sequence
  Description of Artificial Sequence Synthetic primer
acgaaattactttttcaaagccgtctcgagctacatcttttcgtcacattcgaaac

SEQ ID NO: 559 - DNA, 54 bp, Artificial Sequence
  Description of Artificial Sequence Synthetic primer
accaagaacttagtttcgacggatactagtaaaatgtctgactccgcccaaaac

SEQ ID NO: 560 - DNA, 54 bp, Artificial Sequence
  Description of Artificial Sequence Synthetic primer
acgaaattactttttcaaagccgtctcgagctaccatttcaaggaggccttacg

SEQ ID NO: 561 - DNA, 54 bp, Artificial Sequence
  Description of Artificial Sequence Synthetic primer
accaagaacttagtttcgacggatactagtaaaatggaagaatactccgactcc

SEQ ID NO: 562 - DNA, 54 bp, Artificial Sequence
  Description of Artificial Sequence Synthetic primer
acgaaattactttttcaaagccgtctcgagctagaagtgcaaatcttcggaggt

SEQ ID NO: 563 - DNA, 53 bp, Artificial Sequence
  Description of Artificial Sequence Synthetic primer
accaagaacttagtttcgacggatactagtaaaatggaattcactggtgacat

SEQ ID NO: 564 - DNA, 57 bp, Artificial Sequence
  Description of Artificial Sequence Synthetic primer
acgaaattactttttcaaagccgtctcgagctactaaacagttctgttgttcaagtt

SEQ ID NO: 565 - DNA, 52 bp, Artificial Sequence
  Description of Artificial Sequence Synthetic primer
accaagaacttagtttcgacggatactagtaaaatggcgtcctcttcctcac

SEQ ID NO: 566 - DNA, 54 bp, Artificial Sequence
  Description of Artificial Sequence Synthetic primer
acgaaattactttttcaaagccgtctcgagctactcgaatgatctaggcttcgt

SEQ ID NO: 567 - DNA, 50 bp, Artificial Sequence
  Description of Artificial Sequence Synthetic primer
accaagaacttagtttcgacggatactagtaaaatggcctcaaacggctg

SEQ ID NO: 568 - DNA, 56 bp, Artificial Sequence
  Description of Artificial Sequence Synthetic primer
acgaaattactttttcaaagccgtctcgagctagtcgtcaccgattagtgtatcta

SEQ ID NO: 569 - DNA, 85 bp, Artificial Sequence
  Description of Artificial Sequence Synthetic primer
gaatttaagcaggccaacgtccatactgcttaggacctgtgcctggcaagtcgcagattgaagagtttatcattatcaatactgc

SEQ ID NO: 570 - DNA, 19 bp, Artificial Sequence
  Description of Artificial Sequence Synthetic primer
ctcgtaaaagcaaaggtgg

SEQ ID NO: 571 - DNA, 21 bp, Artificial Sequence
  Description of Artificial Sequence Synthetic primer
gtctcgtgcattaagacaggc

SEQ ID NO: 572 - DNA, 25 bp, Artificial Sequence
  Description of Artificial Sequence Synthetic primer
cctgagagttctagatcatggcaag

SEQ ID NO: 573 - DNA, 23 bp, Artificial Sequence
  Description of Artificial Sequence Synthetic primer
tccaggattagatcaaccaattc

SEQ ID NO: 574 - DNA, 22 bp, Artificial Sequence
  Description of Artificial Sequence Synthetic primer
gatttgaaaggcaacaacaatc

SEQ ID NO: 575 - DNA, 22 bp, Artificial Sequence
  Description of Artificial Sequence Synthetic primer
ggacgactaccacttctacgtc

SEQ ID NO: 576 - DNA, 21 bp, Artificial Sequence
  Description of Artificial Sequence Synthetic primer
agtatctgttcttccaggcga

SEQ ID NO: 577 - DNA, 20 bp, Artificial Sequence
  Description of Artificial Sequence Synthetic primer
cttgatggctgacggtatca

SEQ ID NO: 578 - DNA, 25 bp, Artificial Sequence
  Description of Artificial Sequence Synthetic primer
ctcttgatgtcgtccaagttcttac

SEQ ID NO: 579 - DNA, 83 bp, Artificial Sequence
  Description of Artificial Sequence Synthetic primer
ggtatgggtgctaattttcgttagaagcgctggtacaattttctctgtcattgtgacactaagtttatcattatcaatactgc

SEQ ID NO: 580 - DNA, 71 bp, Artificial Sequence
  Description of Artificial Sequence Synthetic primer
gtaaaaataaaatactcctagtccagtaaatataatgcgacactcttgtggaaattactttttcaaagccg

SEQ ID NO: 581 - DNA, 23 bp, Artificial Sequence
  Description of Artificial Sequence Synthetic primer
cctatattattgtaccacattgc

SEQ ID NO: 582 - DNA, 19 bp, Artificial Sequence
  Description of Artificial Sequence Synthetic primer
ctgatgagctcatcgttac

SEQ ID NO: 583 - DNA, 90 bp, Artificial Sequence
  Description of Artificial Sequence Synthetic primer
cgaagaaaacacacttttatagcggaaccgctttctttatttgaattgtcttgttcaccaaggatggatactagtgactacaaggaccac

SEQ ID NO: 584 - DNA, 17 bp, Artificial Sequence
  Description of Artificial Sequence Synthetic primer
cttcttcgtctctgccc

SEQ ID NO: 585 - DNA, 21 bp, Artificial Sequence
  Description of Artificial Sequence Synthetic primer
cggagagctcgtttcaaaatg

SEQ ID NO: 586 - DNA, 87 bp, Artificial Sequence
  Description of Artificial Sequence Synthetic primer
gtagacatactgtatatacacgagggcgtatcgttcaccagaaagaatataaacataacaagataaacatgtaattagttatgtcac

SEQ ID NO: 587 - DNA, 90 bp, Artificial Sequence
  Description of Artificial Sequence Synthetic primer
gagtcctcactctattaatattttcgagtcctcactctgtcgacctcgagggggggcccggtacccaattcgccggccgcaaattaaagc

SEQ ID NO: 588 - DNA, 89 bp, Artificial Sequence
  Description of Artificial Sequence Synthetic primer
gtacgcatgtaacattatactgaaaaccttgcttgagaaggttttgggacgctcgaaggctttaatttgcggccggcgaattgggtacc

SEQ ID NO: 589 - DNA, 90 bp, Artificial Sequence
  Description of Artificial Sequence Synthetic primer
gagtcatagctctttccattacctgaggacgctgagacagttctcaagcctgacattttttatctagattagtgtgtgtatttgtgtttg

SEQ ID NO: 590 - DNA, 87 bp, Artificial Sequence
  Description of Artificial Sequence Synthetic primer
cgagagatttgcaaagggtctcgacgtcaacaaatacacgtcgaaagaaagacaaaagttatccaaaacggatggcgaattgggtac

SEQ ID NO: 591 - DNA, 90 bp, Artificial Sequence
  Description of Artificial Sequence Synthetic primer
ataaaattttcataatagagtcatagctctttccattaccggatgaagcagaaacagttctcaagcctgacatctagattttttcgatgc

SEQ ID NO: 592 - DNA, 16 bp, Artificial Sequence
  Description of Artificial Sequence Synthetic primer
ggaatttgttgtcagc

SEQ ID NO: 593 - DNA, 17 bp, Artificial Sequence
  Description of Artificial Sequence Synthetic primer
gatacccatagcaccac

SEQ ID NO: 594 - protein, 4 aa, Artificial Sequence
  Description of Artificial Sequence Synthetic peptide
EAEA